(19) United States
(12) Patent Application Publication
(10) Pub. No.: US 2024/0269313 A1
(43) Pub. Date: Aug. 15, 2024

(54) CONJUGATES COMPRISING A PHOSPHORUS(V) MOIETY AND A DRUG

(71) Applicant: Tubulis GmbH, Planegg/Martinsried (DE)

(72) Inventors: Marc-André KASPER, Planegg/Martinsried (DE); Philipp OCHTROP, Planegg/Martinsried (DE); Anil Pandurang JAGTAP, Planegg/Martinsried (DE); Swetlana WUNDER, Planegg/Martinsried (DE); Simon VOGT, Planegg/Martinsried (DE); Dominik SCHUMACHER, Planegg/Martinsried (DE); Jonas HELMA-SMETS, Planegg/Martinsried (DE)

(21) Appl. No.: 18/394,303

(22) Filed: Dec. 22, 2023

(30) Foreign Application Priority Data
Dec. 22, 2022 (EP) 22216022.8
Aug. 24, 2023 (EP) 23193215.3

(51) Int. Cl. A61K 47/68; A61K 47/54; A61P 35/00

(52) U.S. Cl. CPC A61K 47/6889; A61K 47/548; A61K 47/68031; A61K 47/68033; A61K 47/68035; A61K 47/68037; A61K 47/6809; A61K 47/6851; A61P 35/00

(57) ABSTRACT

The present invention relates to a conjugate having the formula (1):

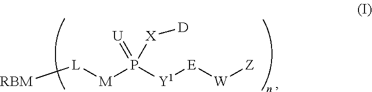

wherein a receptor binding molecule (RBM) is connected with a drug moiety (D), the conjugate comprising a phosphorus(V) moiety. The present invention also relates to intermediates for producing the same, methods of preparing the same, pharmaceutical compositions comprising the same, as well as uses thereof.

### CROSS-REFERENCE TO RELATED APPLICATIONS

The present application claims the right of priority of European patent application EP22216022 filed with the European Patent Office on 22 Dec. 2022, the entire content of which is incorporated herein for all purposes.

The present application claims the right of priority of European patent application EP23193215 filed with the European Patent Office on 24 Aug. 2023, the entire content of which is incorporated herein for all purposes.

### REFERENCE TO AN ELECTRONIC SEQUENCE LISTING

The contents of the electronic sequence listing (406_SeqListing.xml; Size: 9,024 bytes; and Date of Creation: Dec. 19, 2023) is herein incorporated by reference in its entirety.

### TECHNICAL FIELD

The present invention relates to conjugates of a receptor binding molecule with a drug moiety, intermediates for producing the same, methods of preparing the same, pharmaceutical compositions comprising the same, as well as uses thereof.

### BACKGROUND

Antibody-drug conjugates (ADCs) are biotherapeutics that combine cytotoxic molecules with the targeting property of antibodies to specifically kill cancer cells. Sacituzumab govitecan is an ADC, which has been approved for medical use and is marketed under the tradename Trodelvy. In sacituzumab govitecan, the anti-Trop2 antibody sacituzumab, which is also known as hRS7, is connected with the cytotoxic drug SN-38 via a linker denoted as CL2A to form the conjugate hRS7-CL2A-SN-38. The CL2A linker comprises a carbonate moiety to which the drug SN-38 is bound, via its tertiary aliphatic alcohol. However, in vitro cytotoxicity studies using specific and non-specific CL2A-SN-38 conjugates (i.e., comparing a conjugate comprising an antibody that specifically binds the antigen, with a conjugate comprising an antibody that does not bind the antigen) did not show a difference in potency of the specific and-non specific CL2A-SN-38 conjugates, because cleavage to free drug during the assay likely caused the potency of the conjugates to be very similar to that of the free drug (see S. V Govindan et al., “Improving the Therapeutic Index in Cancer Therapy by Using Antibody-Drug Conjugates Designed with a Moderately Cytotoxic Drug”, Mol. Pharmaceutics 2015, 12, 6, 1836-1847, https://doi.org/10.1021/mp5006195.

Another ADC which has been approved for medical use is trastuzumab deruxtecan. Trastuzumab deruxtecan is also known as DS-8201a and is marketed under the tradename Enhertu. Enhertu (DS-8201a) is an anti-Her2 ADC, in which the anti-Her2 antibody trastuzumab is bound via a peptide-containing linker to the cytotoxic drug DXD; see, e.g. Ogitani et al., “DS-8201a, A Novel HER2-Targeting ADC with a Novel DNA Topoisomerase I Inhibitor, Demonstrates a Promising Antitumor Efficacy with Differentiation from T-DM1”, Clinical Cancer Research (22)20, Oct. 15, 2016, pp. 5097-5108 (DOI: 10.1158/1078-0432.CCR-15-2822). However, although Enhertu is an approved and marketed ADC, certain drawbacks still remain. In particular, it has turned out that Enhertu exhibits a comparably low serum stability.

Accordingly, there is an ongoing need for further conjugates which have good properties for pharmaceutical applications. In particular, there is a need for conjugates having a good or improved serum stability, and/or good efficacy.

### SUMMARY

This need is addressed by the subject-matter as defined in the claims and in the embodiments described herein.

Accordingly, the present invention relates to a conjugate having the formula (I):

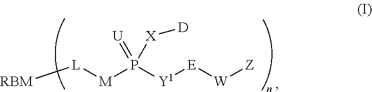

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group;
W is a moiety which, after cleavage of the group Z, is capable of forming a ring together with the spacer E, Y¹ and the phosphorus; and
n is an integer ranging from 1 to 20.

The moiety U, whenever mentioned herein, may be O (oxygen) or S (sulfur). Preferably, U is oxygen. In the present disclosure, whenever at the position of U an O (oxygen) is shown, such as, for example, in formulae (Ia), (Ia1), (Ia2), (Ib), (Ib1), (Ic), (Ic1), (Id), (Id1), (IIa), (IIa1), (IIa2), (IIb), (IIb1), (IIc) or (IIc1), the oxygen can be replaced by S (sulfur). However, for the sake of brevity, the respective formulae comprising S instead of O are not shown. In this context, it is noted again that U is preferably O.

The present invention also relates to a conjugate having the formula (Ia):

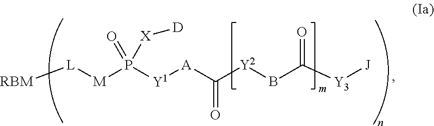

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

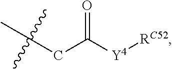

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl; and
n is an integer ranging from 1 to 20.

In some embodiments, the conjugate has formula (Ia1):

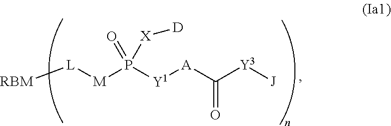

or a pharmaceutically acceptable salt or solvate thereof;
wherein RBM, L, M, X, D, Y¹, A, Y³, J and n are as defined herein.

The present invention also relates to a compound having the formula (II):

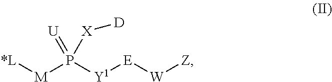

or a pharmaceutically acceptable salt or solvate thereof;
wherein:
L* is a linker capable of forming a covalent attachment to a receptor binding molecule (RBM);
M is O, NR^{M60}, or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group; and
W is a moiety which, after cleavage of the group Z, is capable of forming a ring together with the spacer E, Y¹ and the phosphorus atom.

The present invention also relates to a compound having the formula (IIa):

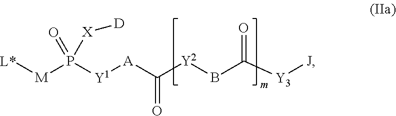

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₃)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

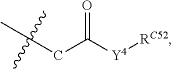

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl.

In some embodiments, the compound has formula (IIa1):

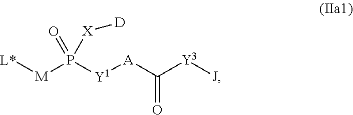

or a pharmaceutically acceptable salt or solvate thereof;
wherein L*, M, X, D, Y¹, A, Y³ and J are as defined herein.

The present invention also relates to a method of preparing a conjugate of formula (I), said method comprising:

### Reacting a Compound of Formula (II)

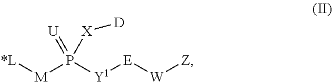

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group; and
W is a moiety which, after cleavage of the group Z, is capable of forming a ring together with the spacer E, Y¹ and the phosphorus;
with a receptor binding molecule (RBM) having a functional group reactive towards L* of a compound of formula (II), thereby forming a covalent bond between the receptor binding molecule (RBM) and the linker (L) to result in a conjugate of formula (I):

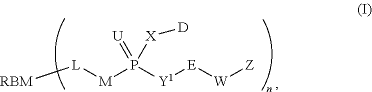

or a pharmaceutically acceptable salt or solvate thereof;
wherein:
RBM is a receptor binding molecule;
L is a linker;
M is O, NR^{M60}, or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group;
W is a moiety which, after cleavage of the group Z, is capable of forming a ring together with the spacer E, Y¹ and the phosphorus; and
n is an integer ranging from 1 to 20.

The present invention also relates to a method of preparing a conjugate of formula (Ia), said method comprising:

### Reacting a Compound of Formula (IIa)

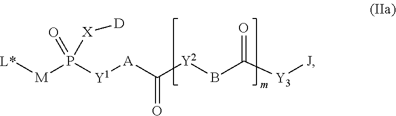

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

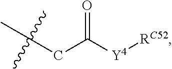

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl; with a receptor binding molecule (RBM) having a functional group reactive towards L* of a compound of formula (IIa), thereby forming a covalent bond between the receptor binding molecule (RBM) and the linker (L) to result in a conjugate of formula (Ia)

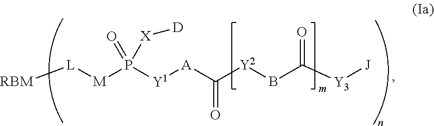

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

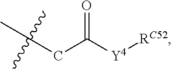

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl; and
n is an integer ranging from 1 to 20.

In some embodiments, the method comprises:

### Reacting a Compound of Formula (IIa1)

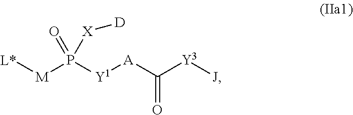

or a pharmaceutically acceptable salt or solvate thereof;
wherein L*, M, X, D, Y¹, A, Y³ and J are as defined herein;
with a receptor binding molecule (RBM) having a functional group reactive towards L* of a compound of formula (IIa1), thereby forming a covalent bond between the receptor binding molecule (RBM) and the linker (L) to result in a conjugate of formula (Ia1)

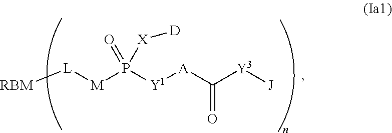

or a pharmaceutically acceptable salt or solvate thereof;
wherein:
RBM, L, M, X, D, Y¹ A, Y³, J and n are as defined herein.

The present invention also relates to a conjugate, or a pharmaceutically acceptable salt or solvate thereof, obtainable or being obtained by a method of of the invention.

The present invention also relates to a pharmaceutical composition comprising a conjugate of the invention.

The present invention also relates to a conjugate of the invention for use in a method of treating a disease. The disease may be cancer.

The present invention also relates to a pharmaceutical composition of the invention for use in a method of treating a disease. The disease may be cancer.

### BRIEF DESCRIPTION OF THE DRAWINGS

FIG. 1 shows an HPLC/UV chromatogram of the compound L-alanine-4-methylbenzylamide.

FIG. 2 shows an HPLC/UV chromatogram of the compound P5(PEG12)-COOH.

FIG. 3 shows an HPLC/UV chromatogram of the compound O-(5-tert.-butoxy-carbonyl-aminopentyl)-N-(L-alanine-tert.-butylester)-O—(SN38)-Phosphoramidate.

FIG. 4 shows an HPLC/UV chromatogram of the compound O-(5-tert.-butoxy-carbonyl-aminopentyl)-N-(4-Methylbenzyl)-O—(SN38)-Phosphoramidate.

FIG. 5 shows an HPLC/UV chromatogram of the compound O-(5-tert.-butoxy-carbonyl-aminopentyl)-N—(L-alanine-4-methylbenzylamide)-O—(SN38)-Phosphoramidate.

FIG. 6 shows an HPLC/UV chromatogram of the compound O-(5-tert.-butoxy-carbonyl-aminopentyl)-N-(L-alanine-L-alanine-tert.-butylester)-O—(SN38)-Phosphoramidate.

FIG. 7 shows an HPLC/UV chromatogram of the compound O-(5-tert.-butoxy-carbonyl-aminopentyl)-N-(β-alanine-L-alanine-tert.-butylester)-O—(SN38)-Phosphoramidate.

FIG. 8 shows an HPLC/UV chromatorgram of the compound O-(5-tert.-butoxy-carbonyl-aminopentyl)-N-(L-alanine-tert.-butylester)-O-(DxD)-Phosphoramidate.

FIG. 9 shows an HPLC/UV chromatogram of the compound O-(5-tert.-butoxy-carbonyl-aminopentyl)-N-(L-alanine-L-Alanine-tert.-butylester)-O-(DxD)-Phosphoramidate.

FIG. 10 shows an HPLC/UV chromatogram of the compound Di-O-(5-tert.-butoxy-carbonyl-aminopentyl)-O—(SN38)-Phosphate.

FIG. 11 shows an HPLC/UV chromatogram of the compound O—(P5(OEt)-amidopentyl)-N-(L-Alanine-tert.-butylester)-O—(SN38)-Phosphoramidate.

FIG. 12 shows an HPLC/UV chromatogram of the compound O—(P5(PEG2)-amidopentyl)-N-(L-Alanine-tert.-butylester)-O—(SN38)-Phosphoramidate.

FIG. 13 shows an HPLC/UV chromatogram of the compound O—(P5(PEG12)-amidopentyl)-N-(L-Alanine-tert.-butylester)-O—(SN38)-Phosphoramidate.

FIG. 14 shows an HPLC/UV chromatogram of the compound O—(P5(PEG12)-amidopentyl)-N-(4-Methylbenzyl)-O—(SN38)-Phosphoramidate.

FIG. 15 shows an HPLC/UV chromatogram of the compound O—(P5(PEG12)-amidopentyl)-N-(L-alanine-L-Alanine-tert.-butylester)-O—(SN38)-Phosphoramidate

FIG. 16 shows an HPLC/UV chromatogram of the compound O—(P5(PEG12)-amidopentyl)-N-(β-alanine-L-alanine-tert.-butylester)-O—(SN38)-Phosphoramidate.

FIG. 17 shows an HPLC/UV chromatogram of the compound O—P5(PEG12)-amidopentyl-Phosphate-O-(5-Aminopentyl)-O-SN38-Phosphate TFA salt.

FIG. 18 shows an HPLC/UV chromatogram of the compound O—(P5(PEG2)-amidopentyl)-N-(L-alanine-L-alanine-tert.-butylester)-O-(DxD)-Phosphoramidate.

FIG. 19 shows an HPLC/UV chromatogram of the compound O—(P5(PEG2)-amidopentyl)-N-(L-alanine-tert.-butylester)-O-(DxD)-Phosphoramidate.

FIG. 20 shows an HPLC/UV chromatogram of the compound O—(P5(PEG2)-amidopentyl)-N-(L-alanine-L-alanine-COOH)—O-(DxD)-Phosphoramidate.

FIG. 21 shows an analytical SEC chromatogram of Trastuzumab. SEC means size exclusion chromatography.

FIG. 22 shows an analytical HIC chromatogram of Trastuzumab. HIC means hydrophobic interaction chromatography.

FIG. 23 shows an analytical SEC chromatogram of Sacituzumab.

FIG. 24 shows an analytical HIC chromatogram of Sacituzumab.

FIG. 25 shows an analytical SEC chromatogram of Palivizumab.

FIG. 26 shows an analytical HIC chromatogram of Palivizumab.

FIG. 27 shows an analytical SEC chromatogram of Sacituzumab-O—P5(PEG2)-amidopentyl-Phosphoramidate-N-(L-alanine-tert.-butylester)-O—SN38.

FIG. 28 shows an analytical HIC chromatogram of Sacituzumab-O—P5(PEG2)-amidopentyl-Phosphoramidate-N-(L-alanine-tert.-butylester)-O—SN38.

FIG. 29 shows an analytical SEC chromatogram of Sacituzumab-O—P5(PEG12)-amidopentyl-Phosphoramidate-N-(L-alanine-tert.-butylester)-O—SN38.

FIG. 30 shows an analytical HIC chromatogram of Sacituzumab-O—P5(PEG12)-amidopentyl-Phosphoramidate-N-(L-alanine-tert.-butylester)-O—SN38.

FIG. 31 shows an analytical SEC chromatogram of Palivizumab-O—P5(PEG12)-amidopentyl-Phosphoramidate-N-(L-alanine-tert.-butylester)-O—SN38.

FIG. 32 shows an analytical HIC chromatogram of Palivizumab-O—P5(PEG12)-amidopentyl-Phosphoramidate-N-(L-alanine-tert.-butylester)-O—SN38.

FIG. 33 shows an analytical SEC chromatogram of Trastuzumab-O—P5(PEG2)-amidopentyl-Phosphoramidate-N-(L-alanine-tert.-butylester)-O-DxD.

FIG. 34 shows an analytical HIC chromatogram of Trastuzumab-O—P5(PEG2)-amidopentyl-Phosphoramidate-N-(L-alanine-tert.-butylester)-O-DxD.

FIG. 35 shows an analytical SEC chromatogram of Palivizumab-O—P5(PEG2)-amidopentyl-Phosphoramidate-N-(L-alanine-tert.-butylester)-O-DxD.

FIG. 36 shows an analytical HIC chromatogram of Palivizumab-O—P5(PEG2)-amidopentyl-Phosphoramidate-N-(L-alanine-tert.-butylester)-O-DxD.

FIG. 37 shows an analytical SEC chromatogram of Trastuzumab-O—P5(PEG2)-amidopentyl-Phosphoramidate-N-(L-alanine-L-alanine-tert.-butylester)-O-DxD.

FIG. 38 shows an analytical HIC chromatogram of Trastuzumab-O—P5(PEG2)-amidopentyl-Phosphoramidate-N-(L-alanine-L-alanine-tert.-butylester)-O-DxD.

FIG. 39 shows an analytical SEC chromatogram of Palivizumab-O—P5(PEG2)-amidopentyl-Phosphoramidate-N-(L-alanine-L-alanine-tert.-butylester)-O-DxD.

FIG. 40 shows an analytical HIC chromatogram of Palivizumab-O—P5(PEG2)-amidopentyl-Phosphoramidate-N-(L-alanine-L-alanine-tert.-butylester)-O-DxD.

FIG. 41 shows an analytical SEC chromatogram of Trastuzumab-O—P5(PEG2)-amidopentyl-Phosphoramidate-N-(L-alanine-L-alanine-COOH)—O-DxD.

FIG. 42 shows an analytical HIC chromatogram of Trastuzumab-O—P5(PEG2)-amidopentyl-Phosphoramidate-N-(L-alanine-L-alanine-COOH)—O-DxD.

FIG. 43 shows an analytical SEC chromatogram of Palivizumab-O—P5(PEG2)-amidopentyl-Phosphoramidate-N-(L-alanine-L-alanine-COOH)—O-DxD.

FIG. 44 shows an analytical HIC chromatogram of Palivizumab-O—P5(PEG2)-amidopentyl-Phosphoramidate-N-(L-alanine-L-alanine-COOH)—O-DxD.

FIGS. 45A and 45B show the results of the direct comparison between ADCs from O—P5(PEG12)-amidopentyl-Phosphoramidate-N-(L-alanine-tert.-butylester)-O—SN38 in accordance with embodiments of the present invention (FIG. 45A) and ADCs from CLA-SN38, which is used in Trodelvy (FIG. 45B). Linkers have been conjugated to Sacituzumab (anti-Trop2) and Trastuzumab (anti Her2, isotype in this setting) and tested on a panel of 4 different cell lines. All cell lines were Trop+ and Her2−. Trastuzumab conjugates serve as isotype control in this setting. Left panel shows the conjugates from O—P5(PEG12)-amidopentyl-Phosphoramidate-N-(L-alanine-tert.-butylester)-O—SN38. Right panel shows the conjugates from CL2A-SN38. Solid lines are Sacituzumab conjugates and dotted lines are Trastuzumab conjugates. Sacituzumab-Cl2A-SN38 is Trodelvy and has been purchased for the experiment. Trastuzumab-CL2A-SN38 has been prepared using the commercially available CL2A linker for the purpose of these experiments.

FIGS. 46A and 46B show the in vitro potency of an ADC comprising Sacituzumab conjugated to the cleavable O—P5(PEG12)-amidopentyl-Phosphoramidate-N-(L-alanine-tert.-butylester)-O—SN38 (solid) in accordance with an embodiment of the invention, and the in vitro potency of an ADC comprising Sacituzumab conjugated to the non cleavable control O—P5(PEG12)-amidopentyl-Phosphoramidate-N-(4-Methylbenzyl)-O—SN38 (dashed).

FIG. 47 shows the results of in vitro potency assays of ADCs from compound O—P5(PEG2)-amidopentyl-Phosphoramidate-N-(L-alanine-tert.-butylester)-O-DxD and the antibodies Trastuzumab (anti-Her2, solid) and Palivizumab (isotype, dashed). The in vitro efficacy on a targeted cell line (SKBR-3, Her2+) and a non-targeted cell line (MDAMB-468, Her2−) is depicted.

FIG. 48 shows the results of in vitro potency assays of ADCs from compound O—P5(PEG2)-amidopentyl-Phosphoramidate-N-(L-alanine-L-alanine-tert.-butylester)-O-DxD and the antibodies Trastuzumab (anti-Her2, solid) and Palivizumab (isotype, dashed). The in vitro efficacy on a targeted cell line (SKBR-3, Her2+) and a non-targeted cell line (MDAMB-468, Her2−) is depicted.

FIG. 49 shows the results of in vitro potency assays of ADCs from compound O—P5(PEG2)-amidopentyl-Phosphoramidate-N-(L-alanine-L-alanine-COOH)—O-DxD and the antibodies Trastuzumab (anti-Her2, solid) and Palivizumab (isotype, dashed). The in vitro efficacy on a targeted cell line (SKBR-3, Her2+) and a non-targeted cell line (MDAMB-468, Her2−) is depicted.

FIGS. 50A and 50B show the results of in vitro potency assays of ADCs from compound O—P5(PEG2)-amidopentyl-Phosphoramidate-N-(L-alanine-L-alanine-COOH)—O-DxD and Trastuzumab (in accordance with an embodiments of the invention, black) and Enhertu (grey). The in vitro efficacy on a targeted cell line (SKBR-3, Her2+) and a non-targeted cell line (MDAMB-468, Her2−) is depicted.

FIG. 51 shows the result of a direct comparison of ADCs from compound O—P5(PEG12)-amidopentyl-Phosphoramidate-N-(L-alanine-tert.-butylester)-O—SN38 conjugated to Sacituzumab (in accordance with an embodiments of the invention, solid) with Trodelvy (dashed) with regard to the stability in the presence of serum at 37° C. Shown is the drug-to-antibody-ratio over time measured by mass spectrometry (MS) after pull-down from rat serum.

FIG. 52 shows the result of a direct comparison of ADCs from compound O—P5(PEG2)-amidopentyl-Phosphoramidate-N-(L-alanine-L-alanine-COOH)—O-DxD conjugated to Trastuzumab (in accordance with an embodiment of the invention, solid) with Enhertu (dashed) with regard to the stability in the presence of serum at 37° C. Shown is the drug-to-antibody-ratio over time measured by mass spectrometry (MS) after pull-down from rat serum.

FIG. 53 shows an HPLC/UV chromatogram of the compound O-(5-tert.-butoxy-carbonyl-aminopentyl)-N—(β-alanine-L-Alanine-tert.-butylester)-O-(DxD)-Phosphoramidate.

FIG. 54 shows an HPLC/UV chromatogram of the compound O-(5-tert.-butoxy-carbonyl-aminopentyl)-N-(γ-Aminobutyric acid-L-Alanine-tert.-butylester)-O-(DxD)-Phosphoramidate.

FIG. 55 shows an HPLC/UV chromatogram of the compound O-(5-tert.-butoxy-carbonyl-aminopentyl)-N-(5-Aminovaleric acid-L-Alanine-tert.-butylester)-O-(DxD)-Phosphoramidate.

FIG. 56 shows an HPLC/UV chromatogram of the compound O-(5-tert.-butoxy-carbonyl-aminopentyl)-N-(L-alanine-tert.-butylester)-O—(OTS-964)-Phosphoramidate.

FIG. 57 shows an HPLC/UV chromatogram of the compound O-(5-tert.-butoxy-carbonyl-aminopentyl)-N-(L-alanine-tert.-butylester)-O-(Ganetespib)-Phosphoramidate.

FIG. 58 shows an HPLC/UV chromatogram of the compound O-(5-tert.-butoxy-carbonyl-aminopentyl)-N-(L-alanine-tert-butylester)-O-(Birabresib)-Phosphoramidate.

FIG. 59 shows an HPLC/UV chromatogram of the compound O-(5-tert.-butoxy-carbonyl-aminopentyl)-N-(L-alanine-L-alanine-tert.-butyl)-O—(SNX-2112)-Phosphoramidate.

FIG. 60 shows an HPLC/UV chromatogram of the compound O-(5-tert.-butoxy-carbonyl-aminopentyl)-N-(L-alanine-L-alanine-tert.-butyl)-O-(Gemcitabine)-Phosphoramidate.

FIG. 61 shows an HPLC/UV chromatogram of the compound O-(5-tert.-butoxy-carbonyl-aminopentyl)-N-(L-alanine-L-alanine-tert.-butyl)-O-(Barasertib)-Phosphoramidate.

FIG. 62 shows an HPLC/UV chromatogram of the compound O—(P5(PEG24)-amidopentyl)-N-(L-alanine-tert.-butylester)-O-(DxD)-Phosphoramidate.

FIG. 63 shows an HPLC/UV chromatogram of the compound O—(P5(PEG24)-amidopentyl)-N-(L-alanine-L-alanine-COOH)—O-(DxD)-Phosphoramidate.

FIG. 64 shows an HPLC/UV chromatogram of the compound O-(6-Maleimidocaproic acid-amidopentyl)-N-(L-alanine-L-alanine-COOH)—O-(DxD)-Phosphoramidate.

FIG. 65 shows an HPLC/UV chromatogram of the compound O-(lodoaceticacid-amidopentyl)-N-(L-alanine-L-alanine-COOH)—O-(DxD)-Phosphoramidate.

FIG. 66 shows an HPLC/UV chromatogram of the compound O—(P5(PEG24)-amidopentyl)-N—(β-alanine-L-alanine-COOH)—O-(DxD)-Phosphoramidate.

FIG. 67 shows an HPLC/UV chromatogram of the compound O—(P5(PEG24)-amidopentyl)-N-(γ-Aminobutyric acid-L-alanine-COOH)—O-(DxD)-Phosphoramidate.

FIG. 68 shows an HPLC/UV chromatogram of the compound O—(P5(PEG24)-amidopentyl)-N-(5-Aminovaleric acid-L-alanine-COOH)—O-(DxD)-Phosphoramidate.

FIG. 69 shows an HPLC/UV chromatogram of the compound O—(P5(PEG24)-amidopentyl)-N-(L-alanine-tert.-butylester)-O—(OTS-964)-Phosphoramidate.

FIG. 70 shows an HPLC/UV chromatogram of the compound O—(P5(PEG24)-amidopentyl)-N-(L-alanine-tert.-butylester)-O-(Ganetespib)-Phosphoramidate.

FIG. 71 shows an HPLC/UV chromatogram of the compound O—(P5(PEG24)-amidopentyl)-N-(L-alanine-tert.-butylester)-O-(Birabresib)-Phosphoramidate.

FIG. 72 shows an HPLC/UV chromatogram of the compound O—(P5(PEG24)-amidopentyl)-N-(L-alanine-L-alanine-tert.-butylester)-O—(SNX-2112)-Phosphoramidate.

FIG. 73 shows an HPLC/UV chromatogram of the compound O—(P5(PEG24)-amidopentyl)-N—(L-alanine-L-alanine)-O—(SNX-2112)-Phosphoramidate.

FIG. 74 shows an HPLC/UV chromatogram of the compound O—(P5(PEG24)-amidopentyl)-N—(L-alanine-L-alanine-tert.-butylester)-O-(Gemcitabine)-Phosphoramidate.

FIG. 75 shows an HPLC/UV chromatogram of the compound O—(P5(PEG24)-amidopentyl)-N—(L-alanine-L-alanine)-O-(Gemcitabine)-Phosphoramidate.

FIG. 76 shows an HPLC/UV chromatogram of the compound O—(P5(PEG24)-amidopentyl)-N—(L-alanine-L-alanine-tert.-butylester)-O-(Barasertib)-Phosphoramidate.

FIG. 77 shows an HPLC/UV chromatogram of the compound O—(P5(PEG24)-amidopentyl)-N—(L-alanine-L-alanine)-O-(Barasertib)-Phosphoramidate.

FIG. 78 shows an analytical SEC chromatogram of Trastuzumab-O—P5(PEG24)-amidopentyl-Phosphoramidate-N-(L-alanine-tert.-butylester)-O-OTS-964.

FIG. 79 shows an analytical HIC chromatogram of Trastuzumab-O—P5(PEG24)-amidopentyl-Phosphoramidate-N-(L-alanine-tert.-butylester)-O-OTS-964.

FIG. 80 shows an analytical SEC chromatogram of Palivizumab-O—P5(PEG24)-amidopentyl-Phosphoramidate-N-(L-alanine-tert.-butylester)-O-OTS-964.

FIG. 81 shows an analytical HIC chromatogram of Palivizumab-O—P5(PEG24)-amidopentyl-Phosphoramidate-N-(L-alanine-tert.-butylester)-O-OTS-964.

FIG. 82 shows an analytical SEC chromatogram of Trastuzumab-O—P5(PEG24)-amidopentyl-Phosphoramidate-N-(L-alanine-tert.-butylester)-O-Ganetespib.

FIG. 83 shows an analytical HIC chromatogram of Trastuzumab-O—P5(PEG24)-amidopentyl-Phosphoramidate-N-(L-alanine-tert.-butylester)-O-Ganetespib.

FIG. 84 shows an analytical SEC chromatogram of Palivizumab-O—P5(PEG24)-amidopentyl-Phosphoramidate-N-(L-alanine-tert.-butylester)-O-Ganetespib.

FIG. 85 shows an analytical HIC chromatogram of Palivizumab-O—P5(PEG24)-amidopentyl-Phosphoramidate-N-(L-alanine-tert.-butylester)-O-Ganetespib.

FIG. 86 shows an analytical SEC chromatogram of Trastuzumab-O—P5(PEG24)-amidopentyl-Phosphoramidate-N-(L-alanine-tert.-butylester)-O-Birabresib.

FIG. 87 shows an analytical HIC chromatogram of Trastuzumab-O—P5(PEG24)-amidopentyl-Phosphoramidate-N-(L-alanine-tert.-butylester)-O-Birabresib.

FIG. 88 shows an analytical SEC chromatogram of Palivizumab-O—P5(PEG24)-amidopentyl-Phosphoramidate-N-(L-alanine-tert.-butylester)-O-Birabresib.

FIG. 89 shows an analytical HIC chromatogram of Palivizumab-O—P5(PEG24)-amidopentyl-Phosphoramidate-N-(L-alanine-tert.-butylester)-O-Birabresib.

FIG. 90 shows an analytical SEC chromatogram of Trastuzumab-O—P5(PEG24)-amidopentyl-Phosphoramidate-N-(L-alanine-L-alanine)-O—SNX-2112.

FIG. 91 shows an analytical HIC chromatogram of Trastuzumab-O—P5(PEG24)-amidopentyl-Phosphoramidate-N-(L-alanine-L-alanine)-O—SNX-2112.

FIG. 92 shows an analytical SEC chromatogram of Palivizumab-O—P5(PEG24)-amidopentyl-Phosphoramidate-N-(L-alanine-L-alanine)-O—SNX-2112.

FIG. 93 shows an analytical HIC chromatogram of Palivizumab-O—P5(PEG24)-amidopentyl-Phosphoramidate-N-(L-alanine-L-alanine)-O—SNX-2112.

FIG. 94 shows an analytical SEC chromatogram of Trastuzumab-O—P5(PEG24)-amidopentyl-Phosphoramidate-N-(L-alanine-L-alanine)-O-Gemcitabine.

FIG. 95 shows an analytical HIC chromatogram of Trastuzumab-O—P5(PEG24)-amidopentyl-Phosphoramidate-N-(L-alanine-L-alanine)-O-Gemcitabine.

FIG. 96 shows an analytical SEC chromatogram of Palivizumab-O—P5(PEG24)-amidopentyl-Phosphoramidate-N-(L-alanine-L-alanine)-O-Gemcitabine.

FIG. 97 shows an analytical HIC chromatogram of Palivizumab-O—P5(PEG24)-amidopentyl-Phosphoramidate-N-(L-alanine-L-alanine)-O-Gemcitabine.

FIG. 98 shows an analytical SEC chromatogram of Trastuzumab-O—P5(PEG24)-amidopentyl-Phosphoramidate-N-(L-alanine-L-alanine)-O-Barasertib.

FIG. 99 shows an analytical HIC chromatogram of Trastuzumab-O—P5(PEG24)-amidopentyl-Phosphoramidate-N-(L-alanine-L-alanine)-O-Barasertib.

FIG. 100 shows an analytical SEC chromatogram of Palivizumab-O—P5(PEG24)-amidopentyl-Phosphoramidate-N-(L-alanine-L-alanine)-O-Barasertib.

FIG. 101 shows an analytical HIC chromatogram of Palivizumab-O—P5(PEG24)-amidopentyl-Phosphoramidate-N-(L-alanine-L-alanine)-O-Barasertib.

FIG. 102 shows an analytical SEC chromatogram of Trastuzumab-O—P5(PEG24)-amidopentyl-Phosphoramidate-N-(L-alanine-tert.-butylester)-O-Dxd.

FIG. 103 shows an analytical HIC chromatogram of Trastuzumab-O—P5(PEG24)-amidopentyl-Phosphoramidate-N-(L-alanine-tert.-butylester)-O-Dxd.

FIG. 104 shows an analytical SEC chromatogram of Palivizumab-O—P5(PEG24)-amidopentyl-Phosphoramidate-N-(L-alanine-tert.-butylester)-O-Dxd.

FIG. 105 shows an analytical HIC chromatogram of Palivizumab-O—P5(PEG24)-amidopentyl-Phosphoramidate-N-(L-alanine-tert.-butylester)-O-Dxd.

FIG. 106 shows an analytical SEC chromatogram of Trastuzumab-O—P5(PEG24)-amidopentyl-Phosphoramidate-N-(L-alanine-L-alanine)-O-Dxd.

FIG. 107 shows an analytical HIC chromatogram of Trastuzumab-O—P5(PEG24)-amidopentyl-Phosphoramidate-N-(L-alanine-L-alanine)-O-Dxd.

FIG. 108 shows an analytical SEC chromatogram of Palivizumab-O—P5(PEG24)-amidopentyl-Phosphoramidate-N-(L-alanine-L-alanine)-O-Dxd.

FIG. 109 shows an analytical HIC chromatogram of Palivizumab-O—P5(PEG24)-amidopentyl-Phosphoramidate-N-(L-alanine-L-alanine)-O-Dxd.

FIG. 110 shows an analytical SEC chromatogram of Trastuzumab-O—P5(PEG24)-amidopentyl-Phosphoramidate-N—(β-alanine-L-alanine)-O-Dxd.

FIG. 111 shows an analytical HIC chromatogram of Trastuzumab-O—P5(PEG24)-amidopentyl-Phosphoramidate-N—(β-alanine-L-alanine)-O-Dxd.

FIG. 112 shows an analytical SEC chromatogram of Palivizumab-O—P5(PEG24)-amidopentyl-Phosphoramidate-N—(β-alanine-L-alanine)-O-Dxd.

FIG. 113 shows an analytical HIC chromatogram of Palivizumab-O—P5(PEG24)-amidopentyl-Phosphoramidate-N—(β-alanine-L-alanine)-O-Dxd.

FIG. 114 shows an analytical SEC chromatogram of Trastuzumab-O—P5(PEG24)-amidopentyl-Phosphoramidate-N-(γ-Aminobutyric acid-L-alanine)-O-Dxd.

FIG. 115 shows an analytical HIC chromatogram of Trastuzumab-O—P5(PEG24)-amidopentyl-Phosphoramidate-N-(γ-Aminobutyric acid-L-alanine)-O-Dxd.

FIG. 116 shows an analytical SEC chromatogram of Palivizumab-O—P5(PEG24)-amidopentyl-Phosphoramidate-N-(γ-Aminobutyric acid-L-alanine)-O-Dxd.

FIG. 117 shows an analytical HIC chromatogram of Palivizumab-O—P5(PEG24)-amidopentyl-Phosphoramidate-N-(γ-Aminobutyric acid-L-alanine)-O-Dxd.

FIG. 118 shows an analytical SEC chromatogram of Trastuzumab-O—P5(PEG24)-amidopentyl-Phosphoramidate-N-(5-Aminovaleric acid-L-alanine)-O-Dxd.

FIG. 119 shows an analytical HIC chromatogram of Trastuzumab-O—P5(PEG24)-amidopentyl-Phosphoramidate-N-(5-Aminovaleric acid-L-alanine)-O-Dxd.

FIG. 120 shows an analytical SEC chromatogram of Palivizumab-O—P5(PEG24)-amidopentyl-Phosphoramidate-N-(5-Aminovaleric acid-L-alanine)-O-Dxd.

FIG. 121 shows an analytical HIC chromatogram of Palivizumab-O—P5(PEG24)-amidopentyl-Phosphoramidate-N-(5-Aminovaleric acid-L-alanine)-O-Dxd.

FIG. 122 shows a head to head comparison of the in vivo efficacy of the ADC O—P5(PEG12)-amidopentyl-phosphoramidate-N-(L-alanine-tert.-butylester)-O—SN38, which comprises a linker in accordance with embodiments of the invention, with the approved ADC Trodelvy.

FIGS. 123A to 123E show the in vitro efficacy of ADCs in accordance with embodiments of the invention for cell killing in various cancer cell lines. Tested ADCs have spacers E of different length, so that after cleavage of the group Z different ring sizes can be formed together with the phosphorus atom.

FIGS. 124A to 124C show the in vitro efficacy of ADCs in accordance with embodiments of the invention that comprise aromatic alcohols as drug moieties for cell killing in various cancer cell lines.

FIG. 125A to 125C show the in vitro efficacy of ADCs in accordance with embodiments of the invention that comprise aliphatic alcohols as drug moieties for cell killing in various cancer cell lines.

FIGS. 126A to 126D show the in vitro efficacy of ADCs in accordance with embodiments of the invention comprising SN38 as drug and the antibodies Datopotamab (Trop2-targeted) and Brentuximab (non-targeted isotype control). The in vitro efficacy on two different Trop2-positive cell lines (MDA-MB-468) and (HCC-78) is depicted. Tested ADCs have spacer groups E of different chemical nature and cleavable groups Z of different chemical nature, respectively.

FIGS. 127A to 127D show the in vitro efficacy of ADCs in accordance with embodiments of the invention comprising SN38 as drug and the antibodies Brentuximab (CD30-targeted) and Datopotamab (non-targeted isotype control). The in vitro efficacy on two different CD30-positive cell lines (L-540) and (SR-786) is depicted. Tested ADCs have spacer groups E of different chemical nature and cleavable groups Z of different chemical nature, respectively.

FIGS. 128A to 128C show the in vitro efficacy of ADCs in accordance with embodiments of the invention comprising SN38 as drug and the antibodies Datopotamab (Trop2-targeted) and Brentuximab (non-targeted isotype control). The in vitro efficacy on two different Trop2-positive cell lines (MDA-MB-468) and (HCC-78) is depicted. Tested ADCs comprise a cyclic group (cyclohexyl) in the linker L and have spacer groups E of different chemical nature and cleavable groups Z of different chemical nature, respectively.

FIGS. 129A to 129C show the in vitro efficacy of ADCs in accordance with embodiments of the invention comprising SN38 as drug and the antibodies Brentuximab (CD30-targeted) and Datopotamab (non-targeted isotype control). The in vitro efficacy on two different CD30-positive cell lines (L-540) and (SR-786) is depicted. Tested ADCs comprise a cyclic group (cyclohexyl) in the linker L and have spacer groups E of different chemical nature and cleavable groups Z of different chemical nature, respectively.

FIGS. 130A to 130D show the in vitro efficacy of ADCs in accordance with embodiments of the invention comprising SN38 as drug and the antibodies Datopotamab (Trop2-targeted) and Brentuximab (non-targeted isotype control). The in vitro efficacy on two different Trop2-positive cell lines (MDA-MB-468) and (HCC-78) is depicted. Tested ADCs comprise various combinations of spacer group E, moiety W and cleavable group Z of different chemical nature,

FIGS. 131A to 130D show the in vitro efficacy of ADCs in accordance with embodiments of the invention comprising SN38 as drug and the antibodies Brentuximab (CD30-targeted) and Datopotamab (non-targeted isotype control). The in vitro efficacy on two different CD30-positive cell lines (L-540) and (SR-786) is depicted. Tested ADCs comprise various combinations of spacer group E, moiety W and cleavable group Z of different chemical nature,

FIGS. 132A and 132B shows the in vitro efficacy of ADCs in accordance with embodiments of the invention comprising DXd as drug and the antibodies Datopotamab (Trop2-targeted) and Brentuximab (non-targeted isotype control). The in vitro efficacy on two different Trop2-positive cell lines (MDA-MB-468) and (HCC-78) is depicted. Tested ADCs comprise various combinations of spacer group E, moiety W and cleavable group Z of different chemical nature,

FIGS. 133A and 133B show the in vitro efficacy of ADCs in accordance with embodiments of the invention comprising DXd as drug and the antibodies Brentuximab (CD30-targeted) and Datopotamab (non-targeted isotype control). The in vitro efficacy on two different CD30-positive cell lines (L-540) and (SR-786) is depicted. Tested ADCs comprise various combinations of spacer group E, moiety W and cleavable group Z of different chemical nature,

FIGS. 134A and 134B show the in vitro efficacy of ADCs in accordance with embodiments of the invention comprising different drugs and variations in moieties X, Y¹ and M. Tested was an ADC comprising SN38 as drug, NH as moiety M, O as moiety Y¹ as well as X, and the antibodies Datopotamab (Trop2-targeted) and Brentuximab (non-targeted isotype control). Also tested was an ADC comprising Exetecan as drug, O as moiety M as well as Y¹, NH as X (to which Exetecan is attached), and the antibodies Datopotamab (Trop2-targeted) and Brentuximab (non-targeted isotype control). The in vitro efficacy on two different Trop2-positive cell lines (MDA-MB-468) and (HCC-78) is depicted.

FIGS. 134C and 134D show the in vitro efficacy of ADCs in accordance with embodiments of the invention comprising different drugs and variations in moieties X, Y¹ and M. Tested was an ADC comprising SN38 as drug, NH as moiety M, O as moiety Y¹ as well as X, and the antibodies Brentuximab (CD30-targeted) and Datopotamab (non-targeted isotype control). Also tested was an ADC comprising Exetecan as drug, O as moiety M as well as Y¹, NH as X (to which Exetecan is attached), and the antibodies Brentuximab (CD30-targeted) and Datopotamab (non-targeted isotype control). The in vitro efficacy on two different CD30-positive cell lines (L-540) and (SR-786) is depicted.

FIGS. 135A and 135B show the in vitro efficacy of ADCs in accordance with embodiments of the invention comprising DXd as drug and the antibodies Datopotamab (Trop2-targeted) and Brentuximab (non-targeted isotype control). The in vitro efficacy on two different Trop2-positive cell lines (MDA-MB-468) and (HCC-78) is depicted. Tested ADCs have a linker L of different chemical nature.

FIGS. 136A and 136B show the in vitro efficacy of ADCs in accordance with embodiments of the invention comprising DXd as drug and the antibodies Brentuximab (CD30-targeted) and Datopotamab (non-targeted isotype control). The in vitro efficacy on two different CD30-positive cell lines (L-540) and (SR-786) is depicted. Tested ADCs have a linker L of different chemical nature.

FIGS. 137A and 137B show the in vitro efficacy of ADCs in accordance with embodiments of the invention comprising different dihydroorotate dehydrogenase (DHODH) inhibitors as drug and the antibodies Datopotamab (Trop2-targeted) and Brentuximab (non-targeted isotype control). The in vitro efficacy on two different Trop2-positive cell lines (MDA-MB-468) and (HCC-78) is depicted. Tested ADCs comprised Bay-2402234 and DHODH-IN-16, respectively.

FIGS. 138A and 138B show the in vitro efficacy of ADCs in accordance with embodiments of the invention comprising different DHODH inhibitors as drug and the antibodies Brentuximab (CD30-targeted) and Datopotamab (non-targeted isotype control). The in vitro efficacy on two different CD30-positive cell lines (SUDHL1) and (Karpas-299) is depicted. Tested ADCs comprised Bay-2402234 and DHODH-IN-16, respectively.

FIG. 139 shows the in vitro efficacy of ADCs in accordance with embodiments of the invention comprising Paclitaxel as drug and the antibodies Trastuzumab (Her2-targeted) and Brentuximab (non-targeted isotype control). The in vitro efficacy on two different HER2-positive cell lines (N87) and (SKBR3) is depicted.

FIGS. 140A to 140C show the in vitro efficacy of ADCs in accordance with embodiments of the invention comprising the HSP90 inhibitor Ganetespib as drug and the antibodies Trastuzumab (Her2-targeted) and Brentuximab or Palivizumab (non-targeted isotype control). The in vitro efficacy on Her2-positive cell (N-87) is depicted. Tested ADCs comprise cleavable groups Z of different chemical nature.

FIGS. 141A to 141C show the in vitro efficacy of ADCs in accordance with embodiments of the invention comprising an elF4E inhibitor as drug. Tested conjugates contained either antibodies Datopotamab (Trop2-targeted) and Brentuximab (non-targeted isotype control on Trop2 positive cell lines) or Brentuximab (CD30-targeted) and Datopotamab (non-targeted isotype control on CD30 positive cell lines). Shown is the in vitro efficacy on Trop2-positive cell line (HCC-78) and on CD30-positive cell line (L-540). Tested ADCs comprise cleavable groups Z of different chemical nature.

FIG. 142 shows an HPLC/UV chromatogram of the compound boc-aminopentane phenyl phosphite.

FIG. 143 shows an HPLC/UV chromatogram of the compound O-(5-tert.-butoxy-carbonyl)-aminopentyl-Phosphoramidate-N-(L-alanine-iso-propylester)-O—SN38.

FIG. 144 shows an HPLC/UV chromatogram of the compound O—P5(PEG24)-amidopentyl-Phosphoramidate-N-(L-alanine-iso-propylester)-O—SN38.

FIG. 145 shows an HPLC/UV chromatogram of the compound O-(5-tert.-butoxy-carbonyl)-aminopentyl-Phosphoramidate-N-(L-alanine-iso-propylester)-O—ON-013100.

FIG. 146 shows an HPLC/UV chromatogram of the compound O—P5(PEG24)-amidopentyl-Phosphoramidate-N-(L-alanine-iso-propylester)-O—ON-013100.

FIG. 147 shows an HPLC/UV chromatogram of the compound O-(5-tert.-butoxy-carbonyl)-aminopentyl-Phosphoramidate-N-(L-alanine-iso-propylester)-O-Ganetespib.

FIG. 148 shows an HPLC/UV chromatogram of the compound O—P5(PEG24)-amidopentyl-Phosphoramidate-N-(L-alanine-iso-propylester)-O-Ganetespib.

FIG. 149 shows an HPLC/UV chromatogram of the compound O-(5-tert.-butoxy-carbonyl)-amidopentyl-Phosphoramidate-N-(glycine-tert.-butylester)-O-4-nitrophenyl.

FIG. 150 shows an HPLC/UV chromatogram of the compound O-(5-tert.-butoxy-carbonyl)-amidopentyl-Phosphoramidate-N-(glycine-tert.-butylester)-O—SN38.

FIG. 151 shows an HPLC/UV chromatogram of the compound O—P5(PEG24)-amidopentyl-Phosphoramidate-N-(L-alanine-iso-propylester)-O—SN38.

FIG. 152 shows an HPLC/UV chromatogram of the compound O-(5-tert.-butoxy-carbonyl)-amidopentyl-Phosphoramidate-N-(2,2-dimethylaminobutyric acid-tert.-butylester)-O-4-nitrophenyl.

FIG. 153 shows an HPLC/UV chromatogram of the compound O-(5-tert.-butoxy-carbonyl)-amidopentyl-Phosphoramidate-N-(2,2-dimethylaminobutyric acid-tert.-butylester)-O—SN38.

FIG. 154 shows an HPLC/UV chromatogram of the compound O—P5(PEG24)-amidopentyl-Phosphoramidate-N-(2,2-dimethylaminobutyric acid-tert.-butylester)-O—SN38.

FIG. 155 shows an HPLC/UV chromatogram of the compound O-5-(5-tert.-butoxy-carbonyl)-amidopentyl-Phosphoramidate-N-(L-alanine-L-alanine-tert.-butylester)-O—SN38.

FIG. 156 shows an HPLC/UV chromatogram of the compound O—P5(PEG24)-amidopentyl-Phosphoramidate-N-(L-alanine-L-alanine)-O—SN38.

FIG. 157 shows an HPLC/UV chromatogram of the compound O-5-tert.-butoxy-carbonyl-amidopentyl-Phosphoramidate-N-(L-alanine-L-alanine-tert.-butylester)-O—ON-013100.

FIG. 158 shows an HPLC/UV chromatogram of the compound O—P5(PEG24)-amidopentyl-Phosphoramidate-N-(L-alanine-L-alanine)-O—ON-013100.

FIG. 159 shows an HPLC/UV chromatogram of the compound O-5-(tert.-butoxy-carbonyl)-amidopentyl-Phosphoramidate-N-(L-alanine-L-alanine-tert.-butylester)-O-Ganetespib.

FIG. 160 shows an HPLC/UV chromatogram of the compound O—P5(PEG24)-amidopentyl-Phosphoramidate-N-(L-alanine-L-alanine)-O-Ganetespib.

FIG. 161 shows an HPLC/UV chromatogram of the compound O-(5-tert.-butoxy-carbonyl)-amidocyclohexyl-Phosphoramidate-N-(L-alanine-L-alanine-tert.-butylester)-O-4-nitrophenyl.

FIG. 162 shows an HPLC/UV chromatogram of the compound O-5-(tert.-butoxy-carbonyl)-amidocyclohexyl-Phosphoramidate-N-(L-alanine-L-alanine-tert.-butylester)-O—SN38.

FIG. 163 shows an HPLC/UV chromatogram of the compound O—P5(PEG24)-amidocyclohexyl-Phosphoramidate-N-(L-alanine-L-alanine)-O—SN38.

FIG. 164 shows an HPLC/UV chromatogram of the compound O-5-(tert.-butoxy-carbonyl)-amidocyclohexyl-Phosphoramidate-N-(L-alanine-tert.-butylester)-O-4-nitrophenyl.

FIG. 165 shows an HPLC/UV chromatogram of the compound O—P5(PEG24)-amidocyclohexyl-Phosphoramidate-N-(L-alanine-tert.-butylester)-O—SN38.

FIG. 166 shows an HPLC/UV chromatogram of the compound O-5-(tert.-butoxy-carbonyl)-amidocyclohexyl-Phosphoramidate-N-(L-alanine-iso-propylester)-O-4-nitrophenyl.

FIG. 167 shows an HPLC/UV chromatogram of the compound O-5-(tert.-butoxy-carbonyl)-amidocyclohexyl-Phosphoramidate-N-(L-alanine-iso-propylester)-O—SN38.

FIG. 168 shows an HPLC/UV chromatogram of the compound O—P5(PEG24)-amidocyclohexyl-Phosphoramidate-N-(L-alanine-iso-propylester)-O—SN38.

FIG. 169 shows an HPLC/UV chromatogram of the compound O-5-(tert.-butoxy-carbonyl)-amidocyclohexyl-Phosphoramidate-N-(2,2-aminobutyric acid-tert.-butylester)-O-4-nitrophenyl.

FIG. 170 shows an HPLC/UV chromatogram of the compound O-5-(tert.-butoxy-carbonyl)-amidocyclohexyl-Phosphoramidate-N-(2,2-aminobutyric acid-tert.-butylester)-O—SN38.

FIG. 171 shows an HPLC/UV chromatogram of the compound O—P5(PEG24)-amidocyclohexyl-Phosphoramidate-N-(2,2-aminobutyric acid-tert.-butylester)-O—SN38.

FIG. 172 shows an HPLC/UV chromatogram of the compound O-5-(tert.-butoxy-carbonyl)-amidopentyl-Phosphoramidate-N-(2-tert.-butyl-disulfide-ethyl)-O-4-nitrophenyl.

FIG. 173 shows an HPLC/UV chromatogram of the compound O-5-(tert.-butoxy-carbonyl)-amidopentyl-Phosphoramidate-N-(2-tert.-butyl-disulfide-ethyl)-SN38.

FIG. 174 shows an HPLC/UV chromatogram of the compound O—P5(PEG24)-amidopentyl-Phosphoramidate-N-(2-tert.-butyl-disulfide-ethyl)-SN38.

FIG. 175 shows an HPLC/UV chromatogram of the compound O-5-(tert.-butoxy-carbonyl)-amidopentyl-Phosphoramidate-N-(2-tert.-butyl-disulfide-ethyl)-DXD.

FIG. 176 shows an HPLC/UV chromatogram of the compound O—P5(PEG24)-amidopentyl-Phosphoramidate-N-(2-tert.-butyl-disulfide-ethyl)-DXD.

FIG. 177 shows an HPLC/UV chromatogram of the compound O-5-(phenylmethoxy-carbonyl)-amidopentyl-Phosphoramidate-N-(2-2-Diethoxy-ethyl)-O-4-nitrophenyl.

FIG. 178 shows an HPLC/UV chromatogram of the compound O-5-(phenylmethoxy-carbonyl)-amidopentyl-Phosphoramidate-N-(2-2-Diethoxy-ethyl)-O-DXD.

FIG. 179 shows an HPLC/UV chromatogram of the compound O—P5(PEG24)-amidopentyl-Phosphoramidate-N-(2-2-Diethoxy-ethyl)-O-DXD.

FIG. 180 shows an HPLC/UV chromatogram of the compound O-5-(tert.-butoxy-carbonyl)-amidopentyl-Phosphoramidate-N-(L-alanine-L-alanine-tert.-butylester)-O-DHODH-IN-16.

FIG. 181 shows an HPLC/UV chromatogram of the compound O—P5(PEG24)-amidopentyl-Phosphoramidate-N-(L-alanine-L-alanine-tert.-butylester)-O-DHODH-IN-16.

FIG. 182 shows an HPLC/UV chromatogram of the compound O-5-(tert.-butoxy-carbonyl)-amidopentyl-Phosphoramidate-N-(L-alanine-L-alanine-tert.-butylester)-O-Roniciclib.

FIG. 183 shows an HPLC/UV chromatogram of the compound O—P5(PEG24)-amidopentyl-Phosphoramidate-N-(L-alanine-L-alanine-tert.-butylester)-O-Roniciclib.

FIG. 184 shows an HPLC/UV chromatogram of the compound O-5-(tert.-butoxy-carbonyl)-amidopentyl-Phosphoramidate-N-(L-alanine-L-alanine-tert.-butylester)-O-Nampt-IN-1.

FIG. 185 shows an HPLC/UV chromatogram of the compound O—P5(PEG24)-amidopentyl-Phosphoramidate-N-(L-alanine-L-alanine-tert.-butylester)-O-Nampt-IN-1.

FIG. 186 shows an HPLC/UV chromatogram of the compound O-5-(tert.-butoxy-carbonyl)-amidopentyl-Phosphoramidate-N-(L-alanine-L-alanine-tert.-butylester)-O-Paclitaxel.

FIG. 187 shows an HPLC/UV chromatogram of the compound O—P5(PEG24)-amidopentyl-Phosphoramidate-N-(L-alanine-L-alanine-tert.-butylester)-O-Paclitaxel.

FIG. 188 shows an HPLC/UV chromatogram of the compound O-5-(tert.-butoxy-carbonyl)-amidopentyl-Phosphoramidate-N-(L-alanine-L-alanine-tert.-butylester)-O-Triptolide.

FIG. 189 shows an analytical SEC chromatogram of Brentuximab-O—P5(PEG24)-amidopentyl-Phosphoramidate-N-(L-alanine-iso-propylerster)-O—SN38.

FIG. 190 shows an analytical HIC chromatogram of Brentuximab-O—P5(PEG24)-amidopentyl-Phosphoramidate-N-(L-alanine-iso-propylerster)-O—SN38.

FIG. 191 shows an analytical SEC chromatogram of Datopotamab-O—P5(PEG24)-amidopentyl-Phosphoramidate-N-(L-alanine-iso-propylerster)-O—SN38.

FIG. 192 shows an analytical HIC chromatogram of Datopotamab-O—P5(PEG24)-amidopentyl-Phosphoramidate-N-(L-alanine-iso-propylerster)-O—SN38.

FIG. 193 shows an analytical SEC chromatogram of Brentuximab-O—P5(PEG24)-amidopentyl-Phosphoramidate-N-(glycine-tert.-butylester)-O—SN38.

FIG. 194 shows an analytical HIC chromatogram of Brentuximab-O—P5(PEG24)-amidopentyl-Phosphoramidate-N-(glycine-tert.-butylester)-O—SN38.

FIG. 195 shows an analytical SEC chromatogram of Datopotamab-O—P5(PEG24)-amidopentyl-Phosphoramidate-N-(glycine-tert.-butylester)-O—SN38.

FIG. 196 shows an analytical HIC chromatogram of Datopotamab-O—P5(PEG24)-amidopentyl-Phosphoramidate-N-(glycine-tert.-butylester)-O—SN38.

FIG. 197 shows an analytical SEC chromatogram of Brentuximab-O—P5(PEG24)-amidopentyl-Phosphoramidate-N-(2,2-dimethylaminobutyric acid-tert.-butylester)-O—SN38.

FIG. 198 shows an analytical HIC chromatogram of Brentuximab-O—P5(PEG24)-amidopentyl-Phosphoramidate-N-(2,2-dimethylaminobutyric acid-tert.-butylester)-O—SN38.

FIG. 199 shows an analytical SEC chromatogram of Datopotamab-O—P5(PEG24)-amidopentyl-Phosphoramidate-N-(2,2-dimethylaminobutyric acid-tert.-butylester)-O—SN38.

FIG. 200 shows an analytical HIC chromatogram of Datopotamab-O—P5(PEG24)-amidopentyl-Phosphoramidate-N-(2,2-dimethylaminobutyric acid-tert.-butylester)-O—SN38.

FIG. 201 shows an analytical SEC chromatogram of Brentuximab-O—P5(PEG24)-amidocyclohexyl-Phosphoramidate-N-(L-alanine-L-alanine)-O—SN38.

FIG. 202 shows an analytical HIC chromatogram of Brentuximab-O—P5(PEG24)-amidocyclohexyl-Phosphoramidate-N-(L-alanine-L-alanine)-O—SN38.

FIG. 203 shows an analytical SEC chromatogram of Datopotamab-O—P5(PEG24)-amidocyclohexyl-Phosphoramidate-N-(L-alanine-L-alanine)-O—SN38.

FIG. 204 shows an analytical HIC chromatogram of Datopotamab-O—P5(PEG24)-amidocyclohexyl-Phosphoramidate-N-(L-alanine-L-alanine)-O—SN38.

FIG. 205 shows an analytical SEC chromatogram of Brentuximab-O—P5(PEG24)-amidocyclohexyl-Phosphoramidate-N-(L-alanine-tert.-butylester)-O—SN38.

FIG. 206 shows an analytical HIC chromatogram of Brentuximab-O—P5(PEG24)-amidocyclohexyl-Phosphoramidate-N-(L-alanine-tert.-butylester)-O—SN38.

FIG. 207 shows an analytical SEC chromatogram of Datopotamab-O—P5(PEG24)-amidocyclohexyl-Phosphoramidate-N-(L-alanine-tert.-butylester)-O—SN38.

FIG. 208 shows an analytical HIC chromatogram of Datopotamab-O—P5(PEG24)-amidocyclohexyl-Phosphoramidate-N-(L-alanine-tert.-butylester)-O—SN38.

FIG. 209 shows an analytical SEC chromatogram of Brentuximab-O—P5(PEG24)-amidocyclohexyl-Phosphoramidate-N-(L-alanine-iso-propylester)-O—SN38.

FIG. 210 shows an analytical HIC chromatogram of Brentuximab-O—P5(PEG24)-amidocyclohexyl-Phosphoramidate-N-(L-alanine-iso-propylester)-O—SN38.

FIG. 211 shows an analytical SEC chromatogram of Datopotamab-O—P5(PEG24)-amidocyclohexyl-Phosphoramidate-N-(L-alanine-iso-propylester)-O—SN38.

FIG. 212 shows an analytical HIC chromatogram of Datopotamab-O—P5(PEG24)-amidocyclohexyl-Phosphoramidate-N-(L-alanine-iso-propylester)-O—SN38.

FIG. 213 shows an analytical SEC chromatogram of Brentuximab-5-Pentylacetamide-Phosphoramidate-N-(L-alanine-L-alanine)-O-DXd.

FIG. 214 shows an analytical HIC chromatogram of Brentuximab-5-Pentylacetamide-Phosphoramidate-N-(L-alanine-L-alanine)-O-DXd.

FIG. 215 shows an analytical SEC chromatogram of Datopotamab-O-Pentylacetamide-Phosphoramidate-N-(L-alanine-L-alanine)-O-DXd.

FIG. 216 shows an analytical HIC chromatogram of Datopotamab-O-Pentylacetamide-Phosphoramidate-N-(L-alanine-L-alanine)-O-DXd.

FIG. 217 shows an analytical SEC chromatogram of Brentuximab-6-(2,5-dioxopyrrolidin-1-yl)-N-(5-hydroxypentyl)hexanamido-Phosphoramidate-N-(L-alanine-L-alanine)-O-DXd.

FIG. 218 shows an analytical HIC chromatogram of Brentuximab-6-(2,5-dioxopyrrolidin-1-yl)-N-(5-hydroxypentyl)hexanamido-Phosphoramidate-N-(L-alanine-L-alanine)-O-DXd.

FIG. 219 shows an analytical SEC chromatogram of Datopotamab-6-(2,5-dioxopyrrolidin-1-yl)-N-(5-hydroxypentyl)hexanamido-Phosphoramidate-N-(L-alanine-L-alanine)-O-DXd.

FIG. 220 shows an analytical HIC chromatogram of Datopotamab-6-(2,5-dioxopyrrolidin-1-yl)-N-(5-hydroxypentyl)hexanamido-Phosphoramidate-N-(L-alanine-L-alanine)-O-DXd.

FIG. 221 shows an analytical SEC chromatogram of Brentuximab-O—P5(PEG24)-amidopentyl-Phosphoramidate-N-(L-alanine-L-alanine)-O-DHODH-IN-16.

FIG. 222 shows an analytical HIC chromatogram of Brentuximab-O—P5(PEG24)-amidopentyl-Phosphoramidate-N-(L-alanine-L-alanine)-O-DHODH-IN-16.

FIG. 223 shows an analytical SEC chromatogram of Datopotamab-O—P5(PEG24)-amidopentyl-Phosphoramidate-N-(L-alanine-L-alanine)-O-DHODH-IN-16.

FIG. 224 shows an analytical HIC chromatogram of Datopotamab-O—P5(PEG24)-amidopentyl-Phosphoramidate-N-(L-alanine-L-alanine)-O-DHODH-IN-16.

FIG. 225 shows an analytical SEC chromatogram of Brentuximab-O—P5(PEG24)-amidopentyl-Phosphoramidate-N-(L-alanine-L-alanine)-O-Roniciclib.

FIG. 226 shows an analytical HIC chromatogram of Brentuximab-O—P5(PEG24)-amidopentyl-Phosphoramidate-N-(L-alanine-L-alanine)-O-Roniciclib.

FIG. 227 shows an analytical SEC chromatogram of Trastuzumab-O—P5(PEG24)-amidopentyl-Phosphoramidate-N-(L-alanine-L-alanine)-O-Roniciclib.

FIG. 228 shows an analytical HIC chromatogram of Trastuzumab-O—P5(PEG24)-amidopentyl-Phosphoramidate-N-(L-alanine-L-alanine)-O-Roniciclib.

FIG. 229 shows an analytical SEC chromatogram of Brentuximab-O—P5(PEG24)-amidopentyl-Phosphoramidate-N-(L-alanine-L-alanine)-O-Nampt-IN-1.

FIG. 230 shows an analytical HIC chromatogram of Brentuximab-O—P5(PEG24)-amidopentyl-Phosphoramidate-N-(L-alanine-L-alanine)-O-Nampt-IN-1.

FIG. 231 shows an analytical SEC chromatogram of Datopotamab-O—P5(PEG24)-amidopentyl-Phosphoramidate-N-(L-alanine-L-alanine)-O-Nampt-IN-1.

FIG. 232 shows an analytical HIC chromatogram of Datopotamab-O—P5(PEG24)-amidopentyl-Phosphoramidate-N-(L-alanine-L-alanine)-O-Nampt-IN-1.

FIG. 233 shows an analytical SEC chromatogram of Brentuximab-O—P5(PEG24)-amidopentyl-Phosphoramidate-N-(L-alanine-L-alanine-tert.-butylester)-O-Paclitaxel.

FIG. 234 shows an analytical HIC chromatogram of Brentuximab-O—P5(PEG24)-amidopentyl-Phosphoramidate-N-(L-alanine-L-alanine-tert.-butylester)-O-Paclitaxel.

FIG. 235 shows an analytical SEC chromatogram of Trastuzumab-O—P5(PEG24)-amidopentyl-Phosphoramidate-N-(L-alanine-L-alanine-tert.-butylester)-O-Paclitaxel.

FIG. 236 shows an analytical HIC chromatogram of Trastuzumab-O—P5(PEG24)-amidopentyl-Phosphoramidate-N-(L-alanine-L-alanine-tert.-butylester)-O-Paclitaxel.

FIG. 237 shows an analytical SEC chromatogram of Brentuximab-O—P5(PEG24)-amidopentyl-Phosphoramidate-N-(L-alanine-L-alanine-tert.-butylester)-O-Triptolide.

FIG. 238 shows an analytical HIC chromatogram of Brentuximab-O—P5(PEG24)-amidopentyl-Phosphoramidate-N-(L-alanine-L-alanine-tert.-butylester)-O-Triptolide.

FIG. 239 shows an analytical SEC chromatogram of Trastuzumab-O—P5(PEG24)-amidopentyl-Phosphoramidate-N-(L-alanine-L-alanine-tert.-butylester)-O-Triptolide.

FIG. 240 shows an analytical HIC chromatogram of Trastuzumab-O—P5(PEG24)-amidopentyl-Phosphoramidate-N-(L-alanine-L-alanine-tert.-butylester)-O-Triptolide.

FIG. 241 shows an analytical SEC chromatogram of Brentuximab-O—P5(PEG24)-amidopentyl-Phosphoramidate-N-(L-alanine-L-alanine-)—O—ON013100.

FIG. 242 shows an analytical HIC chromatogram of Brentuximab-O—P5(PEG24)-amidopentyl-Phosphoramidate-N-(L-alanine-L-alanine-)—O—ON013100.

FIG. 243 shows an analytical SEC chromatogram of Datopotamab-O—P5(PEG24)-amidopentyl-Phosphoramidate-N-(L-alanine-L-alanine-)—O—ON013100.

FIG. 244 shows an analytical HIC chromatogram of Datopotamab-O—P5(PEG24)-amidopentyl-Phosphoramidate-N-(L-alanine-L-alanine-)—O—ON013100.

FIG. 245 shows an analytical SEC chromatogram of Brentuximab-O—P5(PEG24)-amidopentyl-Phosphoramidate-N-(L-alanine-L-alanine)-O-Ganetespib.

FIG. 246 shows an analytical HIC chromatogram of Brentuximab-O—P5(PEG24)-amidopentyl-Phosphoramidate-N-(L-alanine-L-alanine)-O-Ganetespib.

FIG. 247 shows an analytical SEC chromatogram of Datopotamab-O—P5(PEG24)-amidopentyl-Phosphoramidate-N-(L-alanine-L-alanine)-O-Ganetespib.

FIG. 248 shows an analytical HIC chromatogram of Datopotamab-O—P5(PEG24)-amidopentyl-Phosphoramidate-N-(L-alanine-L-alanine)-O-Ganetespib.

FIG. 249 shows an HPLC/UV chromatogram of the P5(PEG24)-COOH.

FIG. 250 shows an analytical SEC chromatogram of Brentuximab.

FIG. 251 shows an analytical HIC chromatogram of Brentuximab.

FIG. 252 shows an analytical SEC chromatogram of Datopotamab.

FIG. 253 shows an analytical HIC chromatogram of Datopotamab.

FIG. 254 shows an HPLC/UV chromatogram of the compound O-(5-tert.-butoxy-carbonyl)-amidopentyl-Phosphoramidate-N-(L-alanine-L-alanine-tert.-butyl ester)-O-BAY-2402234.

FIG. 255 shows an HPLC/UV chromatogram of the compound O-P5-(PEG24)-amidopentyl-Phosphoramidate-N-(L-alanine-L-alanine)-O-BAY-2402234.

FIG. 256 shows an analytical SEC chromatogram of Brentuximab-O—P5(PEG24)-amidopentyl-Phosphoramidate-N-(L-alanine-tert.-butylerster)-O—SN38.

FIG. 257 shows an analytical HIC chromatogram of Brentuximab-O—P5(PEG24)-amidopentyl-Phosphoramidate-N-(L-alanine-tert.-butylerster)-O—SN38.

FIG. 258 shows an analytical SEC chromatogram of Datopotamab-O—P5(PEG24)-amidopentyl-Phosphoramidate-N-(L-alanine-tert.-butylerster)-O—SN38.

FIG. 259 shows an analytical HIC chromatogram of Datopotamab-O—P5(PEG24)-amidopentyl-Phosphoramidate-N-(L-alanine-tert.-butylerster)-O—SN38.

FIG. 260 shows an analytical SEC chromatogram of Trastuzumab-O—P5(PEG24)-amidopentyl-Phosphoramidate-N-(L-alanine-L-alanine)-O-Ganetespib.

FIG. 261 shows an analytical HIC chromatogram of Trastuzumab-O—P5(PEG24)-amidopentyl-Phosphoramidate-N-(L-alanine-L-alanine)-O-Ganetespib.

FIG. 262 shows an analytical SEC chromatogram of Palivizumab-O-P5-(PEG24)-amidopentyl-Phosphoramidate-N-(L-alanine-L-alanine)-O-BAY-2402234.

FIG. 263 shows an analytical HIC chromatogram of Palivizumab-O-P5-(PEG24)-amidopentyl-Phosphoramidate-N-(L-alanine-L-alanine)-O-BAY-2402234.

FIG. 264 shows an analytical SEC chromatogram of Datopotamab-O-P5-(PEG24)-amidopentyl-Phosphoramidate-N-(L-alanine-L-alanine)-O-BAY-2402234.

FIG. 265 shows an analytical HIC chromatogram of Datopotamab-O-P5-(PEG24)-amidopentyl-Phosphoramidate-N-(L-alanine-L-alanine)-O-BAY-2402234.

FIG. 266 shows an HPLC/UV chromatogram of the compound O-(5-tert.-butoxy-carbonyl)-amidopentyl-Phosphoramidate-N-(alanine-tert.-butylester)-O—SN38.

FIG. 267 shows an HPLC/UV chromatogram of the compound O—P5(PEG24)-amidopentyl-Phosphoramidate-N-(L-alanine-tert.-butylester)-O—SN38.

FIG. 268 shows an HPLC/UV chromatogram of the compound O—P5(PEG24)-amidopentyl-Phosphoramidate-N-(2-ethoxy-6-(3R,4S,5S,6S)-(methoxycarbonyl)tetrahydro-2H-pyran-3,4,5-triyl triacetate)-O—SN38.

FIG. 269 shows an HPLC/UV chromatogram of the compound O-(5-tert.-butoxy-carbonyl)-amidopentyl-Phosphoramidate-N-(2-Acetoxy-ethyl)-O-4-nitrophenyl.

FIG. 270 shows an HPLC/UV chromatogram of the compound O-(5-tert.-butoxy-carbonyl)-amidopentyl-Phosphoramidate-N-(2-Acetoxy-ethyl)-O-DXD.

FIG. 271 shows an HPLC/UV chromatogram of the compound O—P5(PEG24)-amidopentyl-Phosphoramidate-N-(2-Acetoxy-ethyl)-O-DXD.

FIG. 272 shows an HPLC/UV chromatogram of the compound O-(5-tert.-butoxy-carbonyl)-amidopentyl-Phosphoramidate-N-(2-Acetamido-ethyl)-O-4-nitrophenyl.

FIG. 273 shows an HPLC/UV chromatogram of the compound O-(5-tert.-butoxy-carbonyl)-amidopentyl-Phosphoramidate-N-(2-Acetamido-ethyl)-O—SN38.

FIG. 274 shows an HPLC/UV chromatogram of the compound O-P5-(PEG24)-amidopentyl-Phosphoramidate-N-(2-Acetamido-ethyl)-O—SN38.

FIG. 275 shows an HPLC/UV chromatogram of the compound O-5-(phenylmethoxy-carbonyl)-amidopentyl-Phosphoramidate-N-(2-2-Diethoxy-ethyl)-O—SN38.

FIG. 276 shows an HPLC/UV chromatogram of the compound O—P5(PEG24)-amidopentyl-Phosphoramidate-N-(2-2-Diethoxy-ethyl)-O—SN38.

FIG. 277 shows an HPLC/UV chromatogram of the compound O-(5-tert.-butoxy-carbonyl)-amidopentyl-Phosphoramidate-O-(lactic acid-iso-propylester)-O-4-nitrophenyl.

FIG. 278 shows an HPLC/UV chromatogram of the compound O-(5-tert.-butoxy-carbonyl)-amidopentyl-Phosphoramidate-O-(lactic acid-iso-propylester)-O—SN38.

FIG. 279 shows an HPLC/UV chromatogram of the compound O-P5-(PEG24)-amidopentyl-Phosphoramidate-O-(lactic acid-iso-propylester)-O—SN38.

FIG. 280 shows an HPLC/UV chromatogram of the compound O-(5-tert.-butoxy-carbonyl)-amidopentyl-Phosphoramidate-O-(glycolic acid-iso-propylester)-N-Exatecan.

FIG. 281 shows an HPLC/UV chromatogram of the compound O-P5-(PEG24)-amidopentyl-Phosphoramidate-O-(glycolic acid-iso-propylester)-N-Exatecan.

FIG. 282 shows an HPLC/UV chromatogram of the compound O-(5-tert.-butoxy-carbonyl)-amidopentyl-Phosphoramidate-N-(L-alanine-tert.-butyl ester)-O-ON-013100.

FIG. 283 shows an HPLC/UV chromatogram of the compound O—P5(PEG24)-amidopentyl-Phosphoramidate-N-(L-alanine-tert.-butyl ester)-O—ON-013100.

FIG. 284 shows an analytical SEC chromatogram of Brentuximab-O—P5(PEG24)-amidopentyl-Phosphoramidate-N-(2-tert.-butyl-disulfide-ethyl)-O—SN38.

FIG. 285 shows an analytical HIC chromatogram of Brentuximab-O—P5(PEG24)-amidopentyl-Phosphoramidate-N-(2-tert.-butyl-disulfide-ethyl)-O—SN38.

FIG. 286 shows an analytical SEC chromatogram of Datopotamab-O—P5(PEG24)-amidopentyl-Phosphoramidate-N-(2-tert.-butyl-disulfide-ethyl)-O—SN38.

FIG. 287 shows an analytical HIC chromatogram of Datopotamab-O—P5(PEG24)-amidopentyl-Phosphoramidate-N-(2-tert.-butyl-disulfide-ethyl)-O—SN38.

FIG. 288 shows an analytical SEC chromatogram of Brentuximab-O—P5(PEG24)-amidopentyl-Phosphoramidate-N-(2-tert.-butyl-disulfide-ethyl)-O-DXD.

FIG. 289 shows an analytical HIC chromatogram of Brentuximab-O—P5(PEG24)-amidopentyl-Phosphoramidate-N-(2-tert.-butyl-disulfide-ethyl)-O-DXD.

FIG. 290 shows an analytical SEC chromatogram of O—P5(PEG24)-amidopentyl-Phosphoramidate-N-(2-tert.-butyl-disulfide-ethyl)-O-DXD.

FIG. 291 shows an analytical HIC chromatogram of O—P5(PEG24)-amidopentyl-Phosphoramidate-N-(2-tert.-butyl-disulfide-ethyl)-O-DXD.

FIG. 292 shows an analytical SEC chromatogram of Brentuximab-O—P5(PEG24)-amidopentyl-Phosphoramidate-N-(2-ethoxy-6-(3R,4S,5S,6S)-(methoxycarbonyl)tetrahydro-2H-pyran-3,4,5-triyl triacetate)-O—SN38.

FIG. 293 shows an analytical HIC chromatogram of Brentuximab-O—P5(PEG24)-amidopentyl-Phosphoramidate-N-(2-ethoxy-6-(3R,4S,5S,6S)-(methoxycarbonyl)tetrahydro-2H-pyran-3,4,5-triyl triacetate)-O—SN38.

FIG. 294 shows an analytical SEC chromatogram of Datopotamab-O—P5(PEG24)-amidopentyl-Phosphoramidate-N-(2-ethoxy-6-(3R,4S,5S,6S)-(methoxycarbonyl)tetrahydro-2H-pyran-3,4,5-triyl triacetate)-O—SN38.

FIG. 295 shows an analytical HIC chromatogram of Datopotamab-O—P5(PEG24)-amidopentyl-Phosphoramidate-N-(2-ethoxy-6-(3R,4S,5S,6S)-(methoxycarbonyl)tetrahydro-2H-pyran-3,4,5-triyl triacetate)-O—SN38.

FIG. 296 shows an analytical SEC chromatogram of Brentuximab-O—P5(PEG24)-amidopentyl-Phosphoramidate-N-(2-Acetoxy-ethyl)-O-DXD.

FIG. 297 shows an analytical HIC chromatogram of Brentuximab-O—P5(PEG24)-amidopentyl-Phosphoramidate-N-(2-Acetoxy-ethyl)-O-DXD.

FIG. 298 shows an analytical SEC chromatogram of Datopotamab-O—P5(PEG24)-amidopentyl-Phosphoramidate-N-(2-Acetoxy-ethyl)-O-DXD.

FIG. 299 shows an analytical HIC chromatogram of Datopotamab-O—P5(PEG24)-amidopentyl-Phosphoramidate-N-(2-Acetoxy-ethyl)-O-DXD.

FIG. 300 shows an analytical SEC chromatogram of Brentuximab-O—P5(PEG24)-amidopentyl-Phosphoramidate-N-(2-Acetamido-ethyl)-O—SN38.

FIG. 301 shows an analytical HIC chromatogram of Brentuximab-O—P5(PEG24)-amidopentyl-Phosphoramidate-N-(2-Acetamido-ethyl)-O—SN38.

FIG. 302 shows an analytical SEC chromatogram of Datopotamab-O—P5(PEG24)-amidopentyl-Phosphoramidate-N-(2-Acetamido-ethyl)-O—SN38.

FIG. 303 shows an analytical HIC chromatogram of Datopotamab-O—P5(PEG24)-amidopentyl-Phosphoramidate-N-(2-Acetamido-ethyl)-O—SN38.

FIG. 304 shows an analytical SEC chromatogram of Brentuximab-O-P5-(PEG24)-amidopentyl-Phosphoramidate-O-(lactic acid-iso-propylester)-O—SN38.

FIG. 305 shows an analytical HIC chromatogram of Brentuximab-O-P5-(PEG24)-amidopentyl-Phosphoramidate-O-(lactic acid-iso-propylester)-O—SN38.

FIG. 306 shows an analytical SEC chromatogram of Datopotamab-O-P5-(PEG24)-amidopentyl-Phosphoramidate-O-(lactic acid-iso-propylester)-O—SN38.

FIG. 307 shows an analytical HIC chromatogram of Datopotamab-O-P5-(PEG24)-amidopentyl-Phosphoramidate-O-(lactic acid-iso-propylester)-O—SN38.

FIG. 308 shows an analytical SEC chromatogram of Brentuximab-O-P5-(PEG24)-amidopentyl-Phosphoramidate-O-(glycolic acid-iso-propylester)-N-Exatecan.

FIG. 309 shows an analytical HIC chromatogram of Brentuximab-O-P5-(PEG24)-amidopentyl-Phosphoramidate-O-(glycolic acid-iso-propylester)-N-Exatecan.

FIG. 310 shows an analytical SEC chromatogram of Datopotamab-O-P5-(PEG24)-amidopentyl-Phosphoramidate-O-(glycolic acid-iso-propylester)-N-Exatecan.

FIG. 311 shows an analytical HIC chromatogram of Datopotamab-O-P5-(PEG24)-amidopentyl-Phosphoramidate-O-(glycolic acid-iso-propylester)-N-Exatecan.

FIG. 312 shows an analytical SEC chromatogram of Trastuzumab-O—P5(PEG24)-amidopentyl-Phosphoramidate-N-(L-alanine-L-alanine-)—O—ON013100.

FIG. 313 shows an analytical HIC chromatogram of Trastuzumab-O—P5(PEG24)-amidopentyl-Phosphoramidate-N-(L-alanine-L-alanine-)—O—ON013100.

FIG. 314 shows an analytical SEC chromatogram of Brentuximab-O—P5(PEG24)-amidopentyl-Phosphoramidate-N-(L-alanine-iso-propylester)-O—ON013100.

FIG. 315 shows an analytical HIC chromatogram of Brentuximab-O—P5(PEG24)-amidopentyl-Phosphoramidate-N-(L-alanine-iso-propylester)-O—ON013100.

FIG. 316 shows an analytical SEC chromatogram of Datopotamab-O—P5(PEG24)-amidopentyl-Phosphoramidate-N-(L-alanine-iso-propylester)-O—ON013100.

FIG. 317 shows an analytical HIC chromatogram of Datopotamab-O—P5(PEG24)-amidopentyl-Phosphoramidate-N-(L-alanine-iso-propylester)-O—ON013100.

FIG. 318 shows an analytical SEC chromatogram of Trastuzumab-O—P5(PEG24)-amidopentyl-Phosphoramidate-N-(L-alanine-iso-propylester)-O—ON013100.

FIG. 319 shows an analytical HIC chromatogram of Trastuzumab-O—P5(PEG24)-amidopentyl-Phosphoramidate-N-(L-alanine-iso-propylester)-O—ON013100.

FIG. 320 shows an analytical SEC chromatogram of Brentuximab-O—P5(PEG24)-amidopentyl-Phosphoramidate-N-(L-alanine-isopropylester)-O-Ganetespib.

FIG. 321 shows an analytical HIC chromatogram of Brentuximab-O—P5(PEG24)-amidopentyl-Phosphoramidate-N-(L-alanine-isopropylester)-O-Ganetespib.

FIG. 322 shows an analytical SEC chromatogram of Datopotamab-O—P5(PEG24)-amidopentyl-Phosphoramidate-N-(L-alanine-isopropylester)-O-Ganetespib.

FIG. 323 shows an analytical HIC chromatogram of Datopotamab-O—P5(PEG24)-amidopentyl-Phosphoramidate-N-(L-alanine-isopropylester)-O-Ganetespib.

FIGS. 324A to 324C show the results of an investigation of the release mechanism using an esterase cleavable construct in accordance with the invention. Depicted are LC/MS spectra of observed reaction products of the construct after incubation with different concentrations of an esterase and of a negative control after incubation in the absence of an esterase. While incubation with esterase lead to traceless release of the drug and formation of a fragment of the construct, the construct remained intact when no esterase was present. Also depicted is the structure of the construct A (O—P5(PEG24)-amidocyclohexyl-Phosphoramidate-N-(L-alanine-iso-propylester)-O—SN38), the intermediate of the esterase cleavage reaction, the released drug B (SN38) and the formed fragment C.

### DETAILED DESCRIPTION

The present invention is described in detail in the following and will also be further illustrated by the appended examples and figures.

### Definitions

Unless otherwise indicated, the term “alkyl” by itself or as part of another term in general refers to a substituted or unsubstituted straight chain or branched, saturated hydrocarbon having the indicated number of carbon atoms; e.g., “—(C₁-C₈)alkyl” or “—(C₁-C₁₀)alkyl” refer to an alkyl group having from 1 to 8 or 1 to 10 carbon atoms, respectively). When the number of carbon atoms is not indicated, the alkyl group may have from 1 to 8 carbon atoms. Representative straight chain —(C₁-C₈)alkyl groups include, but are not limited to, -methyl, -ethyl, -n-propyl, -n-butyl, -n-pentyl, -n-hexyl, -n-heptyl and -n-octyl; branched —(C₁-C₈)alkyl groups include, but are not limited to, -isopropyl, -sec-butyl, -isobutyl, -tert-butyl,-isopentyl, and -2-methylbutyl. In some aspects, an alkyl group may be unsubstituted. Optionally, an alkyl group may be substituted, such as e.g. with one or more groups.

Unless otherwise indicated, the term “alkylene” by itself or as part of another term, in general refers to a substituted or unsubstituted branched or straight chain, saturated hydrocarbon radical of the stated number of carbon atoms, preferably 1-10 carbon atoms (—(C₁-C₁₀)alkylene-) or preferably 1 to 8 carbon atoms (—(C₁-C₈)alkylene-), and having two monovalent radical centers derived by the removal of two hydrogen atoms from the same or two different carbon atoms of a parent alkane. When the number of carbon atoms is not indicated, the alkylene group may have from 1 to 8 carbon atoms. Typical alkylene radicals include, but are not limited to: methylene (—CH₂—), 1,2-ethylene (—CH₂CH₂—), 1,3-n-propylene (—CH₂CH₂CH₂—), and 1,4-n-butylene (—CH₂CH₂CH₂CH₂—). In some aspects, an alkylene group may be unsubstituted. Optionally, an alkylene group may be substituted, such as e.g. with one or more groups.

Unless otherwise indicated, the term “alkenyl” by itself or as part of another term in general refers to a substituted or unsubstituted straight chain or branched, unsaturated hydrocarbon having a double bond and the indicated number of carbon atoms; e.g., “—(C₂-C₈)alkenyl” or “—(C₂-C₁₀)alkenyl” refer to an alkenyl group having from 2 to 8 or 2 to 10 carbon atoms, respectively). When the number of carbon atoms is not indicated, the alkenyl group may have from 2 to 8 carbon atoms. Representative —(C₂-C₈)alkenyl groups include, but are not limited to, -ethenyl, -1-propenyl, -2-propenyl, -1-butenyl, -2-butenyl, -isobutenyl, -1-pentenyl, -2-pentenyl, -3-methyl-1-butenyl, -2-methyl-2-butenyl, and -2,3-dimethyl-2-butenyl. In some aspects, an alkenyl group may be unsubstituted. Optionally, an alkenyl group may be substituted, such as e.g. with one or more groups.

Unless otherwise indicated, the term “alkenylene” by itself of as part of another term, in general refers to a substituted or unsubstituted unsaturated branched or straight chain hydrocarbon radical of the stated number of carbon atoms, preferably 2-10 carbon atoms (—(C₂-C₁₀)alkenylene-) or preferably 2 to 8 carbon atoms (—(C₂-C₈)alkenylene-), and having a double bond, and having two monovalent radical centers derived by the removal of two hydrogen atoms from the same or two different carbon atoms of a parent alkene. When the number of carbon atoms is not indicated, the alkenylene group may have from 2 to 8 carbon atoms. Typical alkenylene radicals include, but are not limited to: -ethenylene-, -1-propenylene-, 2-propenylene-, -1-butenylene-, -2-butenylene-, -isobutenylene-, -1-pentenylene-, -2-pentenylene-, -3-methyl-1-butenylene-, -2-methyl-2-butenylene-, and -2,3-dimethyl-2-butenylene-. In some aspects, an alkenylene group may be unsubstituted. Optionally, an alkenylene group may be substituted, such as e.g. with one or more groups.

Unless otherwise indicated, the term “alkynyl” by itself or as part of another term in general refers to a substituted or unsubstituted straight chain or branched, unsaturated hydrocarbon having a triple bond and the indicated number of carbon atoms; e.g., “—(C₂-C₈)alkynyl” or “—(C₂-C₁₀)alkynyl” refer to an alkynyl group having from 2 to 8 or 2 to 10 carbon atoms, respectively). When the number of carbon atoms is not indicated, the alkynyl group may have from 2 to 8 carbon atoms. Representative —(C₂-C₈)alkynyl groups include, but are not limited to, -acetylenyl, -1-propynyl, -2-propynyl, -1-butynyl, -2-butynyl, -1-pentynyl, -2-pentynyl and -3-methyl-1-butynyl. In some aspects, an alkynyl group may be unsubstituted. Optionally, an alkynyl group may be substituted, such as e.g. with one or more groups.

Unless otherwise indicated, the term “alkynylene” by itself of as part of another term, in general refers to a substituted or unsubstituted, branched or straight chain, unsaturated hydrocarbon radical of the stated number of carbon atoms, preferably 2-10 carbon atoms (—(C₂-C₁₀)alkynylene-) or preferably 2 to 8 carbon atoms (—(C₂-C₈)alkynylene-), and having a triple bond, and having two monovalent radical centers derived by the removal of two hydrogen atoms from the same or two different carbon atoms of a parent alkyne. When the number of carbon atoms is not indicated, the alkynylene group may have from 2 to 8 carbon atoms. Typical alkynylene radicals include, but are not limited to: -ethynylene-, -1-propynylene-, -2-propynylene-, -1-butynylene-, -2-butynylene-, -1-pentynylene-, -2-pentynylene- and -3-methyl-1-butynylene-. In some aspects, an alkynylene group may be unsubstituted. Optionally, an alkynylene group may be substituted, such as e.g. with one or more groups.

Unless otherwise indicated, the term “aryl,” by itself or as part of another term, in general means a substituted or unsubstituted monovalent carbocyclic aromatic hydrocarbon radical of 6 to 20 carbon atoms (preferably 6 to 14 carbon atoms, more preferably 6 to 10 carbon atoms, in very preferred embodiments 6 carbon atoms) derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, and biphenyl. An exemplary aryl group is a phenyl group. In some aspects, an aryl group may be unsubstituted. Optionally, an aryl group may be substituted, such as e.g. with one or more groups.

Unless otherwise indicated, the term “arylene”, by itself or as part of another term, in general is an aryl group as defined above wherein one of the hydrogen atoms of the aryl group is replaced with a bond (i.e., it is divalent) and can be in the para, meta, or ortho orientations as shown in the following structures, with phenyl as the exemplary group:

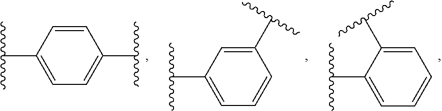

In selected embodiments, the arylene is, e.g., an aryl group as defined above wherein two or more of the hydrogen atoms of the aryl group are replaced with a bond (i.e., the arylene can be trivalent). In some aspects, an arylene group may be unsubstituted. Optionally, an alkynylene group may be substituted, such as e.g. with one or more groups.

Unless otherwise indicated, the term “heterocycle”, “heterocyclyl”, “heterocyclic ring” or the like, by itself or as part of another term, in general refers to a monovalent substituted or unsubstituted aromatic or non-aromatic monocyclic or bicyclic ring system having the indicated number of carbon atoms (e.g., “(C₃-C₈)heterocycle” or “(C₃-C₁₀)heterocycle” refer to a heterocycle having from 3 to 8 or from 3 to 10 carbon atoms, respectively) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a (C₃-C₈)heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl. In some aspects, a heterocycle group may be unsubstituted. Optionally, a heterocycle group may be substituted, such as e.g. with one or more groups.

Unless otherwise indicated, the term “heterocyclo”, “heterocyclyl”, “heterocyclic ring” or the like, by itself or as part of another term, in general refers to a heterocycle group as defined above and having the indicated number of carbon atoms (e.g., (C₃-C₈)heterocycle or (C₃-C₁₀)heterocycle) wherein one of the hydrogen atoms of the heterocycle group is replaced with a bond (i.e., it is divalent). In selected embodiments, the heterocyclo is, e.g., a heterocycle group as defined above wherein two or more of the hydrogen atoms of the heterocycle group are replaced with a bond (i.e., the heterocyclo can be trivalent). In some aspects, a heterocyclo, heterocyclyl or heterocyclic ring may be unsubstituted. Optionally, a heterocyclo, heterocyclyl or heterocyclic ring may be substituted, such as e.g. with one or more groups.

Unless otherwise indicated, the term “carbocycle”, “carbocyclyl”, “carbocyclic ring” or the like, by itself or as part of another term, in general refers to a monovalent, substituted or unsubstituted aromatic or non-aromatic monocyclic or bicyclic carbocyclic ring system having the indicated number of carbon atoms (e.g., “(C₃-C₈)carbocycle” or “(C₃-C₁₀)carbocycle” refer to a carbocycle having from 3 to 8 or from 3 to 10 carbon atoms, respectively) derived by the removal of one hydrogen atom from a ring atom of a parent ring system. As illustrative but non-limiting examples the carbocycle may be a 3-, 4-, 5-, 6-, 7- or 8-membered carbocycle. The term “carbocycle”, “carbocyclyl”, “carbocyclic ring” or the like may also include cycloalkyl, such as for example (C₃-C₈)cycloalkyl, in particular 3-, 4-, 5-, 6-, 7- or 8-membered cycloalkyl. The term “carbocycle”, “carbocyclyl”, “carbocyclic ring” or the like may also include cycloalkenyl, such as for example (C₅-C₈)cycloalkenyl, in particular 5-, 6-, 7- or 8-membered cycloalkenyl. Representative (C₃-C₈)carbocycles include, but are not limited to, phenyl, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. In some aspects, a carbocycle may be unsubstituted. Optionally, a carbocycle may be substituted, such as e.g. with one or more groups.

Unless otherwise indicated, the term “carbocyclo”, “carbocyclyl”, “carbocyclic ring” or the like, by itself or as part of another term, in general refers to a carbocycle group as defined above having the indicated number of carbon atoms (e.g., “(C₃-C₈)carbocyclo” or “(C₃-C₁₀)carbocyclo” refer to a carbocyclo or carbocyclic ring having from 3 to 8 or from 3 to 10 carbon atoms, respectively), wherein another of the hydrogen atoms of the carbocycle groups is replaced with a bond (i.e., it is divalent). The term “carbocyclo”, “carbocyclyl”, “carbocyclic ring” or the like may also include cycloalkyl, such as for example (C₃-C₈)cycloalkyl, and cycloalkenyl, such as for example (C₅-C₈)cycloalkenyl. In selected embodiments, the carbocyclo or carbocyclic ring is, e.g., a carbocycle group as defined above, wherein two or more of the hydrogen atoms of the carbocycle group are replaced with a bond (i.e., the carbocyclo, carbocyclyl or carbocyclic ring can be trivalent). In some aspects, a carbocyclo, carbocyclyl or carbocyclic ring may be unsubstituted. Optionally, a carbocyclo, carbocyclyl or carbocyclic ring may be substituted, such as e.g. with one or more groups.

The term “halogen” or “halo”, unless defined otherwise, in general refers to elements of the 7^{th} main group; preferably fluorine, chlorine, bromine and iodine; more preferably fluorine, chlorine and bromine; even more preferably, fluorine and chlorine.

The term “substituted”, “optionally substituted”, “optionally may be substituted” or the like, unless otherwise indicated, in general means that one or more hydrogen atoms can be each independently replaced with a substituent. Typical substituents include, but are not limited to, —X, —R, —O—, —OR, —SR, —S—, —NR₂, —NR₃, ═NR, —CX₃, —CN, —OCN, —SCN, —N═C═O, —NCS, —NO, —NO₂, ═N₂, —N₃, —NRC(═O)R, —C(═O)R, —C(═O)NR₂, —SO₃—, —SO₃H, —S(═O)₂R, —OS(═O)₂OR, —S(═O)₂NR, —S(═O)R, —OP(═O)(OR)₂, —P(═O)(OR)₂, —PO₄³—, —PO₃H2, —C(═O)R, —C(═O)X, —C(═S)R, —CO₂R, —CO₂, —C(═S)OR, —C(═O)SR, —C(═S)SR, —C(═O)NR₂, —C(═S)NR₂, or —C(═NR)NR₂. R can be the same or different, are independently selected from (C₁-C₈)alkyl, (C₁-C₈)alkylene(C₆-C₁₀)aryl or (C₆-C₁₀)aryl, optionally two R substituents can together form a 3 to 8-membered ring.

The term “leaving group”, as used herein, in general denotes a moiety, e.g. an atom or a group of atoms, which is capable to detach from a main or residual part of a substrate during a reaction or elementary step of a reaction. In particular, a leaving group can be replaced by another moiety, e.g. an atom or a group of atoms, during a substitution reaction. The substituation reaction may be, for example, a nucleophilic substitution.

The term “aliphatic or aromatic residue”, or “aliphatic residue” or “aromatic residue”, or the like, as used herein, in general refers to an aliphatic substituent, such as e.g. but not limited to an alkyl residue, which, however, can be optionally substituted by further aliphatic and/or aromatic substituents. As non-limiting examples an aliphatic residue can be a nucleic acid, an enzyme, a co-enzyme, a nucleotide, an oligonucleotide, a monosaccharide, a polysaccharide, a polymer, a fluorophore, optionally substituted benzene, etc., as long as the direct link of such a molecule to the core structure (in case of R⁸⁰, e.g., the link to the oxygen atom bound to the phosphorus; or in case of the drug moiety (D), e.g., the link to the group X bound to the phosphorus) is aliphatic. An aromatic residue is a substituent, wherein the direct link to the core structure is part of an aromatic system, e.g., an optionally substituted phenyl or triazolyl or pyridyl or nucleotide; as non-limiting example if the direct link of the nucleotide to the core structure is for example via a phenyl-residue. The term “aromatic residue”, as used herein, also includes a heteroaromatic residue.

The term “peptide” or “polypeptide”, unless otherwise indicated, in general refers to an organic compound comprising two or more amino acids covalently joined by peptide bonds (amide bond). Peptides may be referred to with respect to the number of constituent amino acids, i.e., a dipeptide contains two amino acid residues, a tripeptide contains three, etc. Peptides containing ten or fewer amino acids may be referred to as oligopeptides, while those with more than ten amino acid residues, e.g. with up to about 30 amino acid residues, are polypeptides.

The term “amino acid”, as used herein, in general refers to an organic compound having a —CH(NH₃)—COOH group. In one embodiment, the term “amino acid” refers to a naturally occurring amino acid. As illustrative examples, naturally occurring amino acids include arginine, lysine, aspartic acid, glutamic acid, glutamine, asparagine, histidine, serine, threonine, tyrosine, cysteine, methionine, tryptophan, alanine, isoleucine, leucine, phenylalanine, valine, proline and glycine. However, the term in its broader meaning also encompasses non-naturally occurring amino acids.

Amino acids and peptides according to the disclosure can also be modified at functional groups. Non limiting examples are saccharides, e.g., N-Acetylgalactosamine (GaINAc), or protecting groups, e.g., Fluorenylmethoxycarbonyl (Fmoc)-modifications or esters.

The term “antibody”, as used herein, is intended to refer to immunoglobulin molecules, preferably comprised of four polypeptide chains, two heavy (H) chains and two light (L) chains which are typically inter-connected by disulfide bonds. Each heavy chain is comprised of a heavy chain variable region (abbreviated herein as VH) and a heavy chain constant region. The heavy chain constant region can comprise e.g. three domains CH1, CH2 and CH3. Each light chain is comprised of a light chain variable region (abbreviated herein as VL) and a light chain constant region. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs arranged from amino-terminus to carboxy-terminus e.g. in the following order: FR1, CDR1, FR2, CDR2, FR3, CDR3, FR4.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA1, and IgA2. A preferred class of immunoglobulins for use in the present invention is IgG.

The heavy-chain constant domains that correspond to the different classes of antibodies are called [alpha], [delta], [epsilon], [gamma], and [mu], respectively. The subunit structures and three-dimensional configurations of different classes of immunoglobulins are well known. As used herein antibodies are conventionally known antibodies and functional fragments thereof.

A “human” antibody or antigen-binding fragment thereof is in general defined as one that is not chimeric (e.g., not “humanized”) and not from (either in whole or in part) a non-human species. A human antibody or antigen-binding fragment thereof can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained there from. Another example of a human antibody or antigen-binding fragment thereof is one that is encoded by a nucleic acid isolated from a library of antibody sequences of human origin (e.g., such library being based on antibodies taken from a human natural source).

A “humanized antibody” or humanized antigen-binding fragment thereof is in general defined herein as one that is (i) derived from a non-human source (e.g., a transgenic mouse which bears a heterologous immune system), which antibody is based on a human germline sequence; (ii) where amino acids of the framework regions of a non-human antibody are partially exchanged to human amino acid sequences by genetic engineering or (iii) CDR-grafted, wherein the CDRs of the variable domain are from a non-human origin, while one or more frameworks of the variable domain are of human origin and the constant domain (if any) is of human origin.

A “chimeric antibody” or antigen-binding fragment thereof is in general defined herein as one, wherein the variable domains are derived from a non-human origin and some or all constant domains are derived from a human origin.

The term “monoclonal antibody” as used herein in general refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. The term monoclonal antibody specifically includes chimeric, humanized and human antibodies.

“Binding affinity” or “affinity” in general refers to the strength of the total sum of non-covalent interactions between a single binding site of a molecule and its binding partner. Unless indicated otherwise, as used herein, “binding affinity” refers to intrinsic binding affinity which reflects a 1:1 interaction between members of a binding pair (e.g. an antibody and an antigen). The dissociation constant “K_{D}” is commonly used to describe the affinity between a molecule (such as an antibody) and its binding partner (such as an antigen) i.e. how tightly a ligand binds to a particular protein. Ligand-protein affinities are influenced by non-covalent intermolecular interactions between the two molecules. Affinity can be measured by common methods known in the art, including those described herein. In one embodiment, the “K_{D}” or “K_{D} value” according to this invention is measured by using surface plasmon resonance assays using suitable devices including but not limited to Biacore instruments like Biacore T100, Biacore T200, Biacore 2000, Biacore 4000, a Biacore 3000 (GE Healthcare Biacore, Inc.), or a ProteOn XPR36 instrument (Bio-Rad Laboratories, Inc.).

The term “antibody drug conjugate” or abbreviated ADC is well known to a person skilled in the art, and, as used herein, in general refers to the linkage of an antibody or an antigen binding fragment thereof with a drug, such as a chemotherapeutic agent, a toxin, an immunotherapeutic agent, an imaging probe, and the like.

The term “small molecule” as used herein in general denotes an organic molecule comprising at least two carbon atoms, having a molecular weight in the range between 100 and 2000 Dalton, preferably between 100 and 1000 Dalton, and optionally including one or two metal atoms. Optionally, a small molecule may also contain one or more heteroatom(s), such as, for example, N, O, S, P and/or halogen.

The present disclosure also relates to a “pharmaceutically acceptable salt”. Any pharmaceutically acceptable salt can be used. In particular, the term “pharmaceutically acceptable salt” refers to a salt of a conjugate or compound of the invention that is pharmaceutically acceptable and that possesses the desired pharmacological activity of the parent compound. In particular, such salts have low toxicity and may be inorganic or organic acid addition salts and base addition salts. Specifically, such salts include, but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, purely by way of example, sodium, potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like. A counterion or anionic counterion can be used in a quaternary amine to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, and the like), and carboxylate ions (e.g., acetate, ethanoate, propanoate, benzoate, glycerate, lactate, tartrate, glycolate, and the like).

As used herein, the term “solvate” may refer to an aggregate that comprises one or more molecules of a conjugate or compound described herein with one or more molecules of solvent. The solvent may be water, in which case the solvate may be a hydrate. Alternatively, the solvent may be an organic solvent. Thus, the conjugates or compounds of the present disclosure may exist as a hydrate, including a monohydrate, dihydrate, hemihydrate, sesquihydrate, trihydrate, tetrahydrate and the like, as well as the corresponding solvated forms. The compounds of the invention may be true solvates, while in other cases, the compounds of the invention may merely retain adventitious water or be a mixture of water plus some adventitious solvent.

### Conjugate of Formula (I)

The present invention relates to a conjugate having the formula (I):

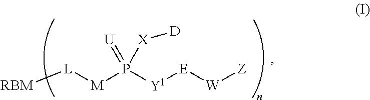

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group;
W is a moiety which, after cleavage of the group Z, is capable of forming a ring together with the spacer E, Y¹ and the phosphorus; and
n is an integer ranging from 1 to 20.

The moiety U, whenever mentioned herein, may be O (oxygen) or S (sulfur).

Preferably, U is oxygen. Accordingly, in preferred embodiments, the conjugate has the following structure:

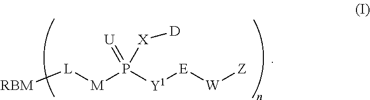

In the present disclosure, whenever at the position of U an O (oxygen) is shown, such as, for example, in formulae (I), (Ia), (Ia1), (Ia2), (Ib), (Ib1), (Ic), (Ic1), (Id), (Id1), (Ie), (Ie1), (If), (If1), (II), (IIa), (IIa1), (IIa2), (IIb), (IIb1), (IIc), (IIc1), (IId1), (IIe), (IIe1), (IIf) or (IIf1) the oxygen can be replaced by S (sulfur). For the sake of brevity, the respective formulae comprising S instead of O are not shown. In this context, it is noted again that U is preferably O.

Conjugates of formula (I) comprise a receptor binding molecule (RBM) such as, for example, an antibody, which is connected to a drug moiety (D) via a linker and a phosphorus(V) moiety having the structure:

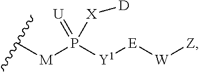

wherein:
M is as described herein;
U is O or S;
X is O, S or NR^{X10};
D is a drug moiety;
Y¹ is NR^{A20}, O, S, or CR^{A21}R^{A22};
E is a spacer;
Z is a cleavable group; and
W is a moiety which, after cleavage of the group Z, is capable of forming a ring together with the spacer E, Y¹ and the phosphorus. It has been found that conjugates of formula (I) have advantageous properties, as shown in the following.

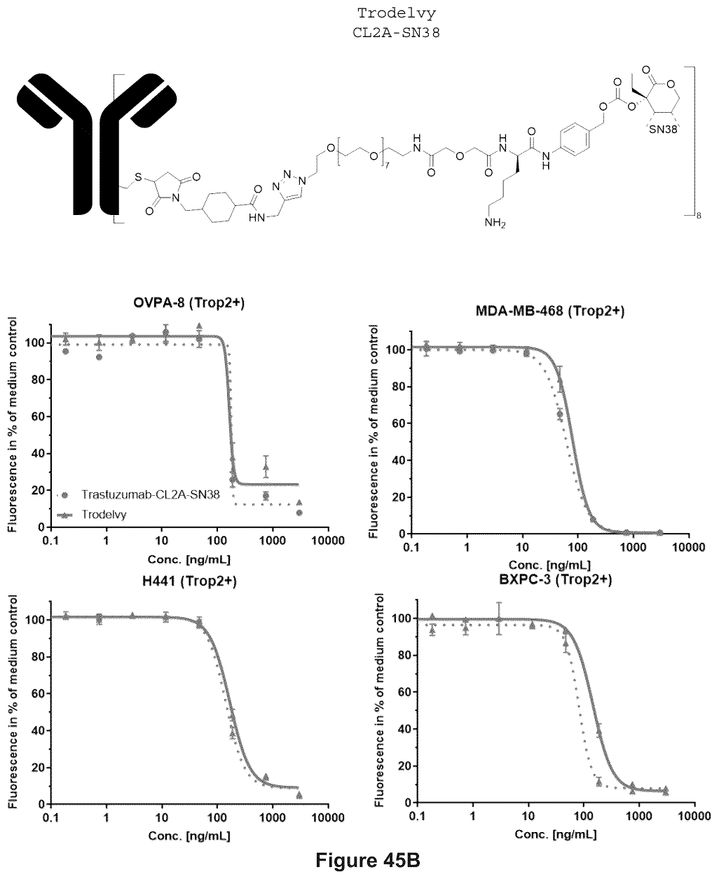

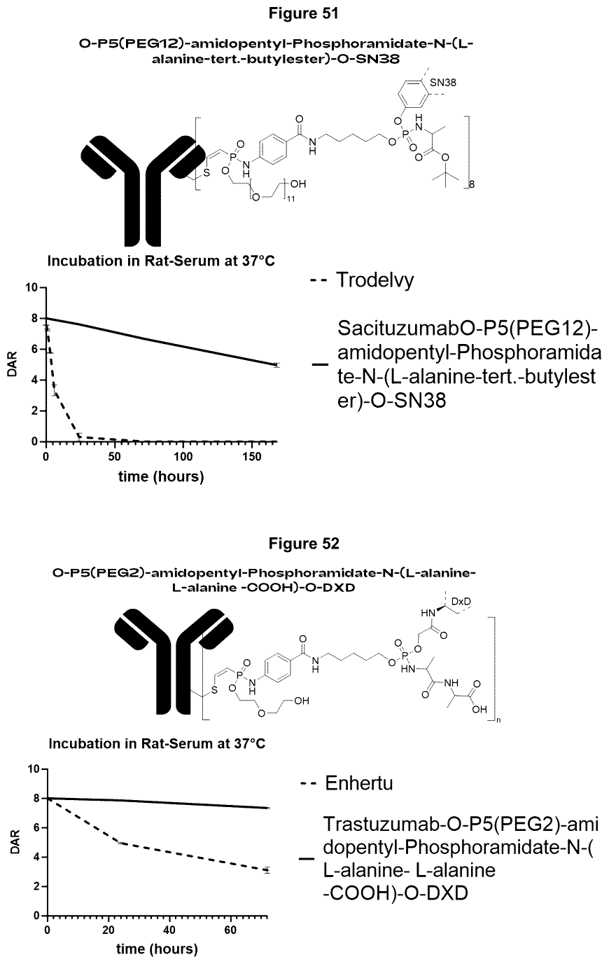

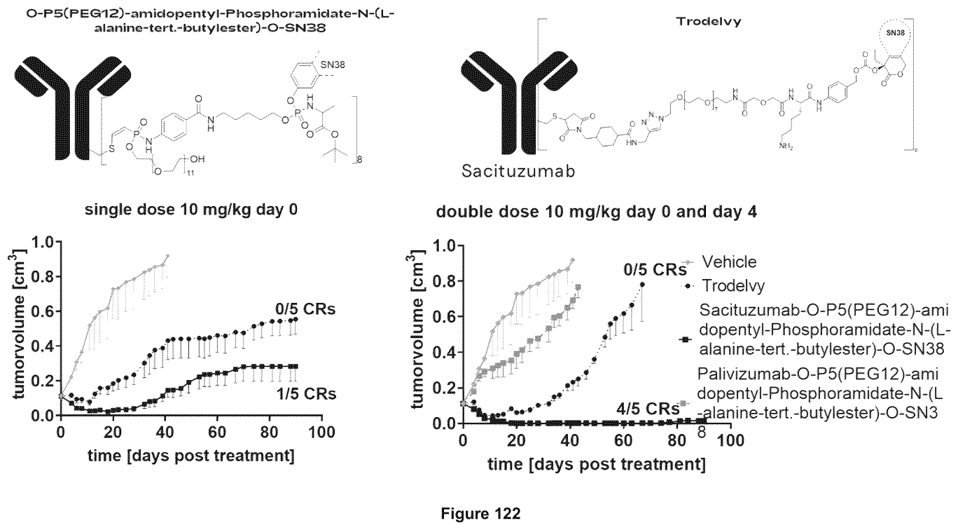

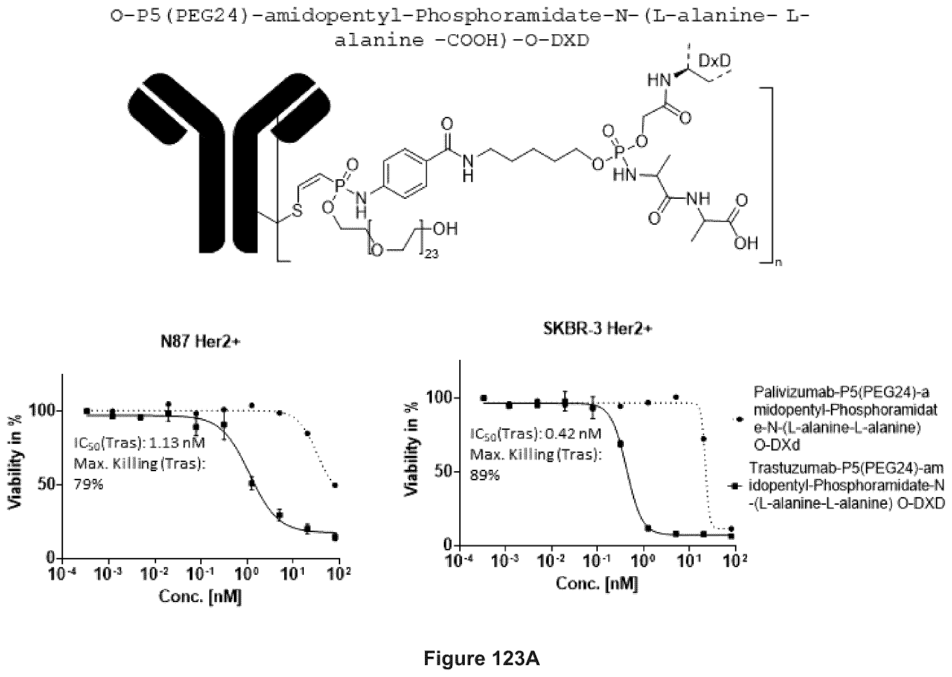

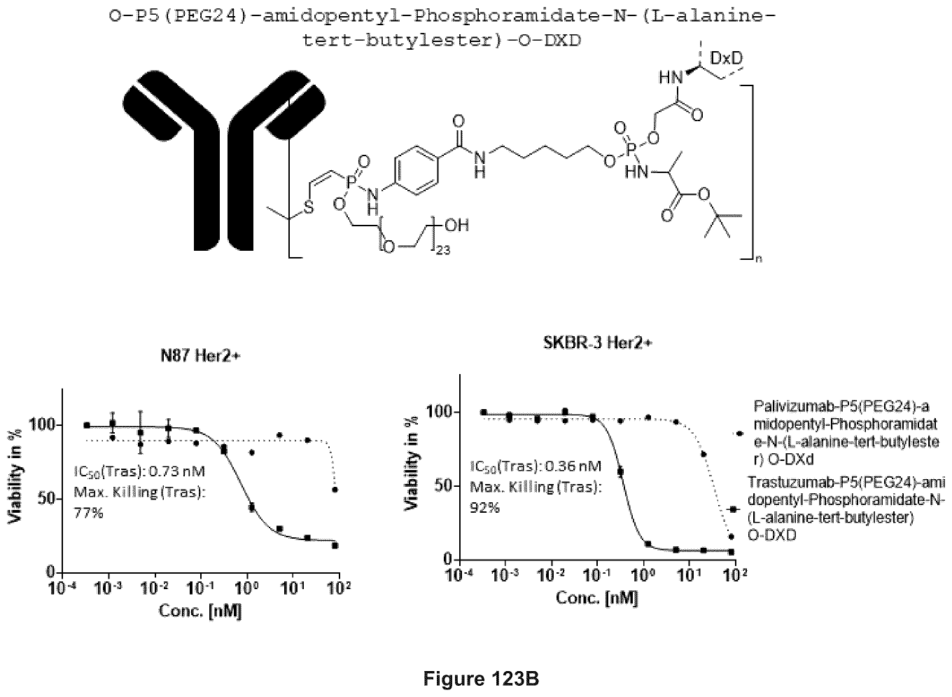

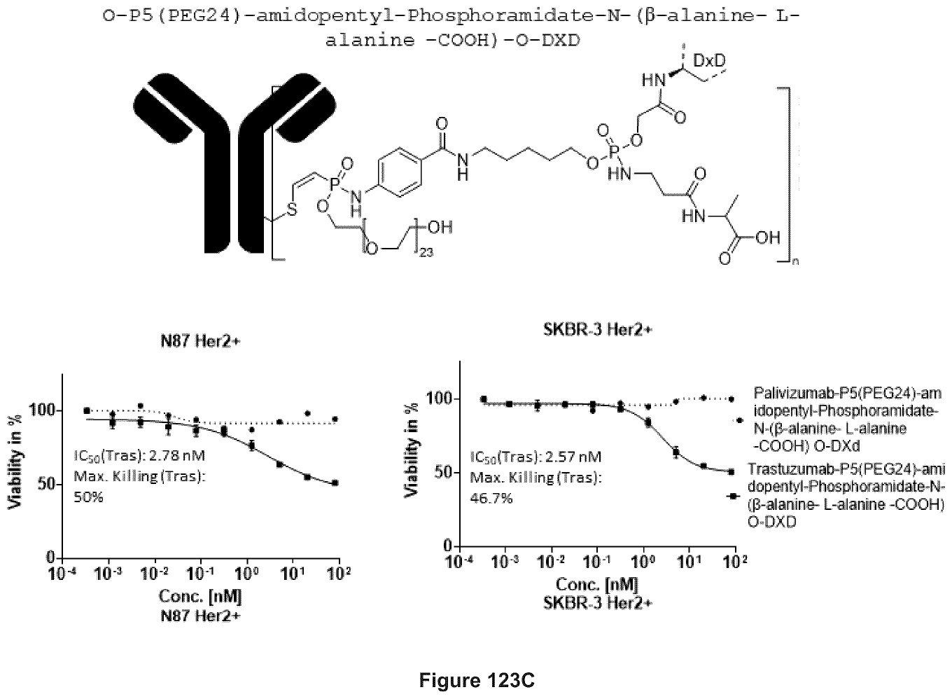

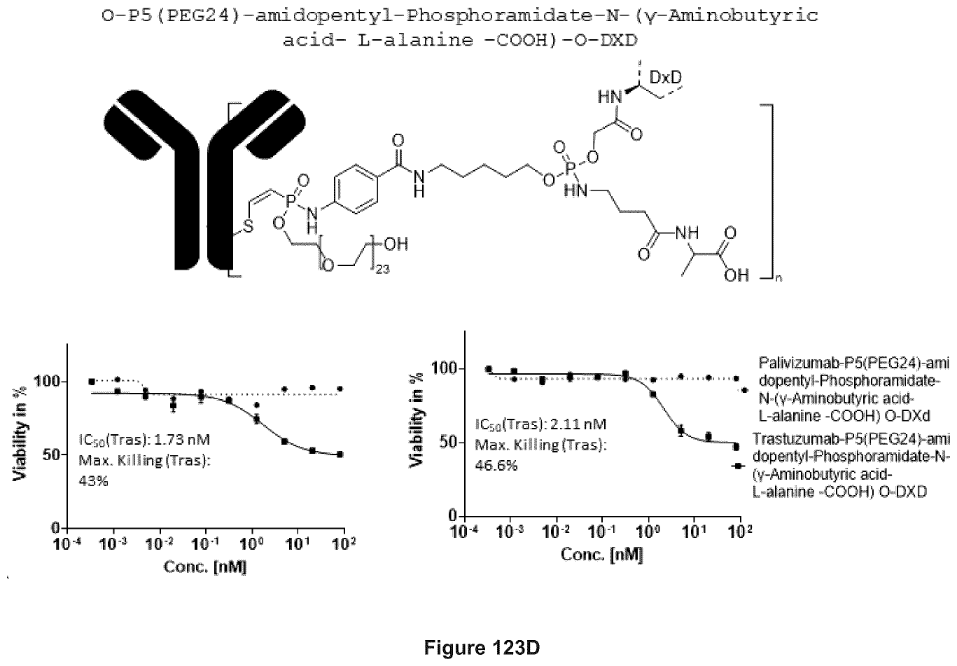

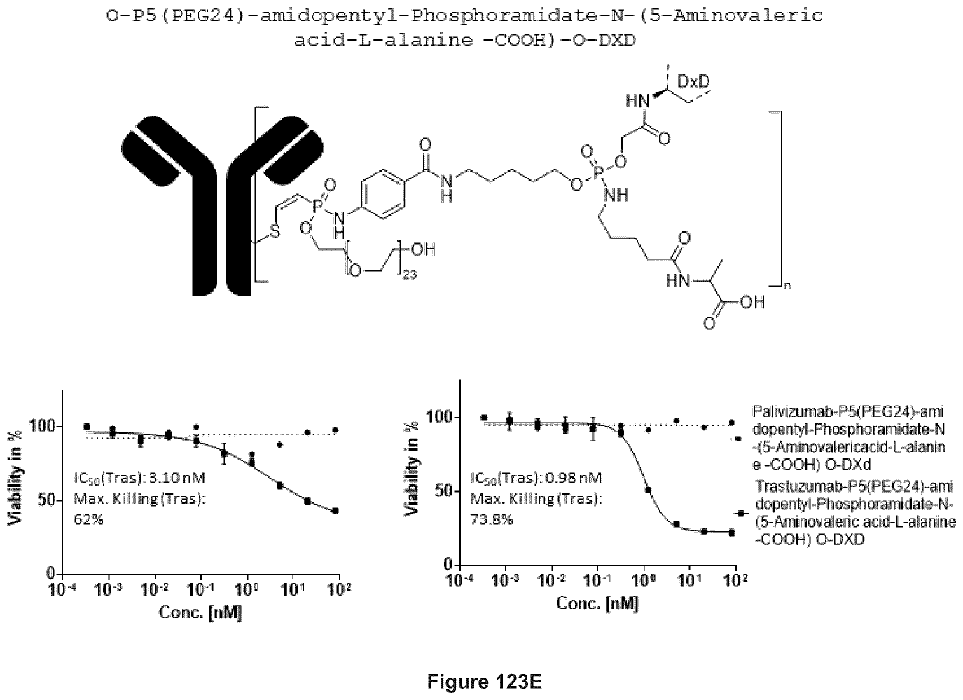

Conjugates of formula (I) having a phosphorus(V) moiety as described herein show a good cytotoxicity, which is selective for the cell line which is targeted by the receptor binding molecule, such as an antibody (Example 3, and FIGS. 45, 46, 47, 48, 49 and 50). Therefore, conjugates of formula (I) allow for targeted delivery of the drug that is mediated by the specific binding of the receptor binding molecule, such as an antibody. Conjugates of formula (I) having a phosphorus(V) moiety as described herein can even achieve an improved potency in comparison to the commercial product Trodelvy, when the same antibody (Sacituzumab) and drug (SN38) is used and only the CL2A linker of Trodelvy is exchanged by a linker in accordance with embodiments of the invention. (Example 3 and FIG. 45). In particular, conjugates of formula (I) show an excellent serum stability, which exceeds the stability of the commercial conjugates Trodelvy and Enhertu (Example 4 and FIGS. 51 and 52). Conjugates of formula (I) also show an excellent efficacy in vivo, for example when compared with Trodelvy (Example 5 and FIG. 122; for comparison with Trodelvy, the same antibody (Sacituzumab) and drug (SN38) is used, and the CL2A linker of Trodelvy is exchanged by a linker in accordance with embodiments of the invention). Good efficacy has been also shown for different structures of the linker and the drugs, which allows for a broad applicability of the technology described herein (Examples 6, 7 and 8; and FIGS. 123A to 123E, 124A to 124C and 125A to 125C). In sum, the inventors have surprisingly found that conjugates of the present invention exhibit excellent properties which make them useful as pharmaceuticals, including an enhanced serum stability, and excellent in vitro and in vivo efficacy. It is noted that conjugates, which comprise a phosphorus(V) moiety, are e.g. described in J.C. Kern et al., “Discovery of pyrophosphate diesters as tunable, soluble and biorthogonal linkers for site-specific antibody-drug conjugates”, J. Am. Chem. Soc. 2016, 138, 4, 1430-1445 (https://doi.org/10.1021/jacs.5b12547); P. Brandish et. al., “Development of Anti-CD74 Antibody-Drug Conjugates to Target Glucocorticoids to Immune Cells”, Bioconjugate Chem. 2018, 29, 7, 2357-2369 (https://doi.org/10.1021/acs.bioconjchem.8b00312); J. C. Kern et al., “Novel Phosphate Modified Cathepsin B Linkers: Improving Aqueous Solubility and Enhancing Payload Scope of ADCs”, Bioconjugate Chem. 2016, 27, 9, 2081-2088 (https://doi.org/10.1021/acs.bioconjchem.6b00337); WO 2018/041985; WO 2019/170710; and WO 2022/223783. A methylene alkoxy carbamate unit has been described for targeted delivery of hydroxy group-containing drugs by R. V. Kolakowski et al., “The Methylene Alkoxy Carbamate Self-Immolative Unit: Utilization for the Targeted Delivery of Acohol-Containing Paylods with Antibody-Drug Conjugates”, Angew. Chem. Int. Ed. 2016, 55, 28, 7948-7951 (https://doi.org/10.1002/anie.201601506).

Without wishing to be bound by theory, the inventors believe that the mechanism of drug release from a conjugate of formula (I) is as provided in the following. As described herein, a conjugate of formula (I) comprises a moiety

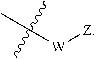

The group Z is a cleavable group (or, in other words, a removable group or a splittable group). In this context, the term “cleavable group” in particular means that the bond between the groups W and Z is cleavable so that the group Z is capable to be split off from the group W. Accordingly, the bond between the groups W and Z is susceptible to cleavage, such as, for example, enzymatic cleavage, acid-induced cleavage, photo-induced cleavage, or disulfide bond cleavage, preferably at conditions under which the drug moiety and/or the receptor binding molecule remains active. As explained, the bond between the groups W and Z is susceptible to enzymatic cleavage. Enzymatic cleavage includes, but is not limited to, protease-induced cleavage, peptidase-induced cleavage, esterase-induced cleavage, thioesterase-induced cleavage, glycosidase-induced cleavage (such as, e.g., glucuronidase-induced cleavage), phosphatase-induced cleavage, and sulfatase-induced cleavage. Moieties

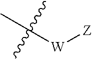

wherein Z is a cleavable group, as comprised in a conjugate of formula (I), are known to a person skilled in the art and can be readily selected; illustrative but non-limiting examples may include ester groups, thioester groups, amide groups, glycosides, disulfides; phosphates and sulfates. The group Z may be any suitable group, such as, for example, an optionally substituted aliphatic residue or an optionally substituted aromatic residue. An illustrative but non-limiting example for

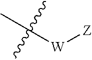

is an ester group

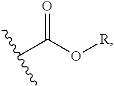

wherein R may be, e.g., an alkyl group such as isopropyl; an ester group can be hydrolyzed, for example, by an esterase to give a carboxylic acid or carboxylate, i.e.

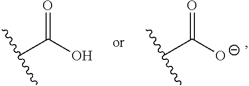

and an alcohol

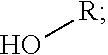

the cleavage of an ester group by hydrolysis may occur inside of a cell also without assistance of an enzyme; accordingly, the cleavable group Z is

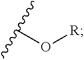

and after cleavage of the group Z, the moiety W is a carboxylic acid or carboxylate, i.e.

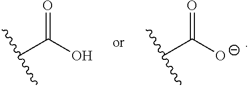

According to another illustrative but non-limiting example,

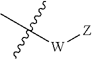

is a thioester group

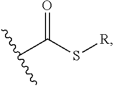

wherein R may be, e.g., an alkyl group such as isopropyl; a thioester group can be hydrolyzed by a thioesterase to give a carboxylic acid or carboxylate, i.e.

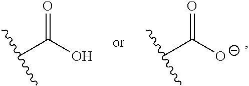

and a thiol

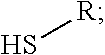

accordingly, the cleavable group Z is

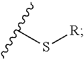

and after cleavage of the group Z, the moiety W is a carboxylic acid or carboxylate, i.e.

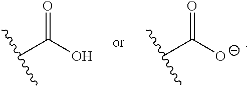

According to another illustrative but non-limiting example,

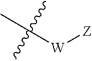

is an amide group, e.g.

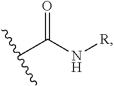

wherein R may be, e.g., an alkyl group, or an amino acid, or a peptide; an amide group can be hydrolyzed by a peptidase or protease to give a carboxylic acid or carboxylate, i.e.

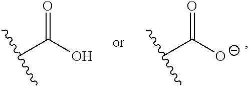

and an amine

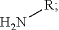

accordingly, the cleavable group Z is

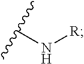

and after cleavage of the group Z, the moiety W is a carboxylic acid or carboxylate, i.e.

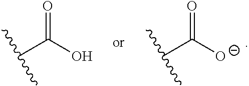

According to another illustrative but non-limiting example,

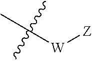

is a glycoside

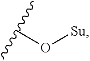

wherein Su is a sugar moiety; a glycoside can be hydrolyzed by a glycosidase to give an alcohol

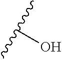 [SMILES: *O]

and a sugar

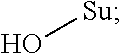

accordingly, the cleavable group Z is Su; and after cleavage of the group Z, the moiety W is a hydroxy group

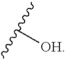 [SMILES: *O]

According to another illustrative but non-limiting example,

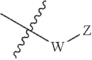

is a disulfide

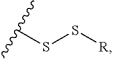

wherein R may be, e.g., an alkyl group such as isopropyl; a disulfide bond can be enzymatically reduced to give two thiols

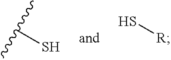

accordingly, the cleavable group Z is

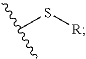

and after cleavage of the group Z, the moiety W is a thiol group

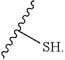 [SMILES: *S]

According to another illustrative but non-limiting example,

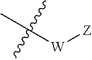

is an amide group, e.g.

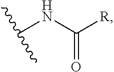

wherein R may be, e.g., an alkyl group, or an amino acid, or a peptide; an amide group can be hydrolyzed by a peptidase or protease to give an amine

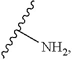 [SMILES: *N]

and a carboxylic acid

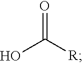

accordingly, the cleavable group Z is

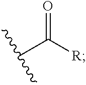

and after cleavage of the group Z, the moiety W is an amine

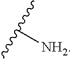 [SMILES: *N]

According to another illustrative but non-limiting example,

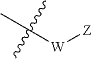

is an ester group, e.g.

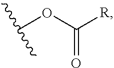

wherein R may be, e.g., an alkyl group such as methyl, ethyl or isopropyl; an ester group can be hydrolyzed, for example, by an esterase to give an alcohol

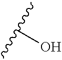 [SMILES: *O]

and a carboxylic acid

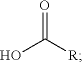

accordingly, the cleavable group Z is

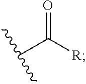

and after cleavage of the group Z, the moiety W is an alcohol

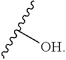 [SMILES: *O]

Such cleavage mechanisms can take place at the target site, for example, as known by a person skilled in the art, such cleavage mechanisms can take place in a cell after internalization of the conjugate.

As described herein, “W is a moiety which, after cleavage of the group Z, is capable of forming a ring together with the spacer E, Y¹ and the phosphorus” of a conjugate of formula (I). Preferably, W is a moiety which, after cleavage of the group Z, is capable of forming a four- to seven-membered ring together with the spacer E, Y¹ and the phosphorus. More preferably, W is a moiety which, after cleavage of the group Z, is capable of forming a five- or six-membered ring. Thus, without wishing to be bound by theory, it is assumed that, on a mechanistic level, the group W, after cleavage of the group Z, performs an intramolecular attack on the phosphorus atom. The moiety

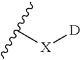

is then released from the conjugate, i.e. the drug is liberated. A possible mechanistic sequence comprising cleavage of the group Z followed by intramolecular attack of W on the phosphorus atom in intermediate A, and release of the drug moiety (X-D) may be depicted as follows:

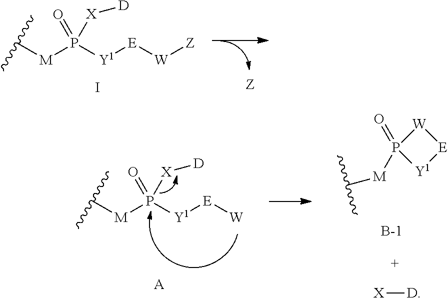

However, other pathways for the further reaction of intermediate A may occur. For example, another possible mechanistic sequence is assumed to also include cleavage of the group Z, followed by intramolecular attack of W on the phosphorus atom in intermediate A; release of the moiety

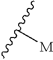

and hydrolysis of intermediate B-2 may lead to intermediate C-1, from which the drug moiety (X-D) can be released under intracellular conditions, with the aid of, for example, hydrolysis or phosphordiesterases; this possible mechanism is depicted in the following:

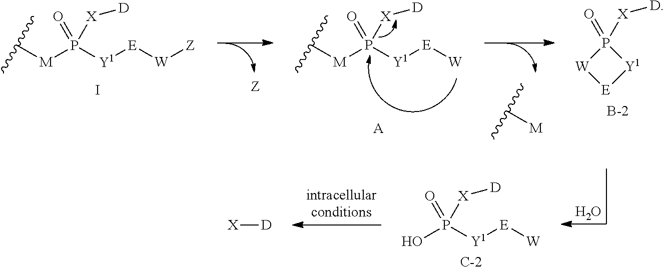

As a further possible alternative, the assumed mechanism may, again, involve cleavage of the group Z followed by intramolecular attack of W on the phosphorus atom in intermediate A; transient formation of a ring comprising W, the phosphorus atom, Y¹ and E may occur; release of Y¹ from A to give intermediate B-3, hydrolysis to give intermediate C-3, and release of the drug moiety (X-D) under intracellular conditions, with the aid of, for example, hydrolysis or phosphordiesterases, may be the further steps, as depicted in the following:

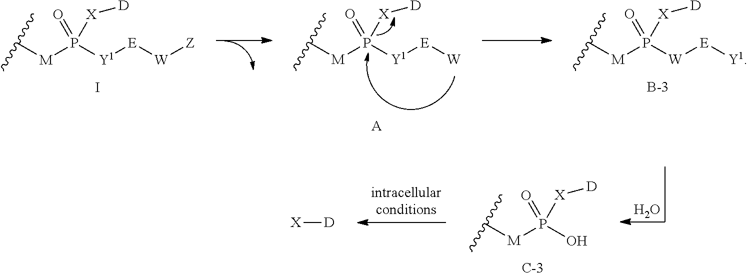

In accordance with the above explanations on the possible release mechanism, the moiety W, after cleavage of the group Z, may be capable to perform a nucleophilic attack on the phosphorus atom. Accordingly, after cleavage of the group Z, the moiety W is in particular nucleophilic. In this regard, moieties W described herein above, after cleavage of the group Z, i.e.

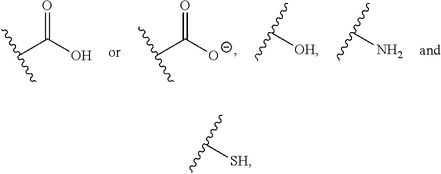

are nucleophilic, as readily appreciated by a person skilled in the art. The moiety

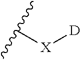

can be considered as a leaving group. Thus, the moiety

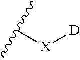

can be considered as a releasable moiety. The moiety

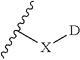

can be capable to be released after cleavage of the group Z. The moiety

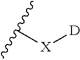

can be released upon or after attack of the group W, after cleavage of the group Z, onto the phosphorus. In particular, the moiety

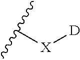

can be capable to be released after cleavage of the group Z and formation of a ring, preferably a four- to seven-membered ring, more preferably a five- or six membered ring, comprising W, the spacer E, Y¹ and the phosphorus. Accordingly, without wishing to be bound by theory, in the proposed possible mechanistic sequences, the reaction of A to give B-1, B-2 or B-3 can be considered as an intramolecular nucleophilic substitution. In particular, the group Z can be cleaved at a target site to initiate a reaction that leads to release of the moiety X-D.

As can be seen, as found herein, all the proposed mechanisms are in accordance with proposed mechanism as it is described with regard to prodrugs of nucleoside analogs, in which the hydroxy groups of monophosphate or monophosphonate groups are masked; see, e.g., Y. Mehellou et al., “The ProTide Prodrug Technology: From the Concept to the Clinic”, J. Med. Chem. 2018, 61, 2211-2226 (DOI: 10.1021/acs.jmedchem.7b00734).

As also described herein, the moiety W, after cleavage of the group Z, is capable of forming a ring together with the spacer E, Y¹, and the phosphorus. Preferably, the moiety W, after cleavage of the group Z, is capable of forming a four- to seven-membered ring together with the spacer E, Y¹ and the phosphorus. More preferably, the moiety W, after cleavage of the group Z, is capable of forming a five- or six-membered ring together with the spacer E, Y¹ and the phosphorus. As described herein, Y¹ is NR^{A20}, O, S or CR^{A21}R^{A22}, wherein R^{A20}, R^{A21} and R^{A22} are as described herein. A person skilled in the art is able to readily select the groups W (or the moiety

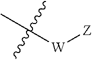

before cleavage of the group Z) and E to give a suitable ring size together with Y¹ and the phosphorus. The term “spacer”, when used herein, in general refers to a chemical group or moiety that serves to connect the groups Y¹ and W. The spacer E is not particularly limited as long as it is suitable for forming a ring (preferably a four- to seven-membered ring, more preferably a five- or six-membered ring), together with Y¹, W and the phosphorus. As illustrative, non-limiting example, E may be a suitable alkylene group which comprises one, two, three or four main chain atoms, such as, for example

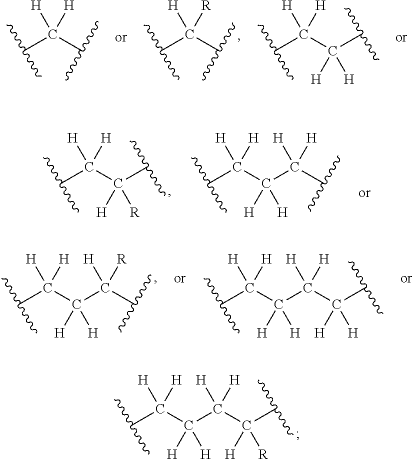

the group R indicates that the alkylene group may be optionally substituted; it is noted that the number and positions of the optional substituents may vary and can be readily adjusted by a person skilled in the art, as may be needed; it is also possible that two substituents of the alkylene group may form a ring. Also, the spacer E may contain cyclic moieties. Thus, as a further illustrative but non-limiting example, the spacer E may be

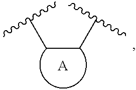

which may be an optionally substituted four- to seven-membered, preferably five- or six-membered, carbocyclic or heterocyclic ring; or, as a more specific example, the spacer E may be

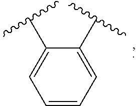 [SMILES: *c1ccccc1*]

Optionally, the spacer E may contain one or more heteroatoms, such as, for example, O, N or S, and/or may be optionally substituted. Further examples for the moieties Y¹, E, W and Z are described herein.

The group Y¹, whenever mentioned herein, is selected from the group consisting of NR^{A20}, O, S, or CR^{A21}R^{A22}, wherein R^{A20}, R^{A21} and R^{A22} are as defined herein. Accordingly, R^{A20} may be selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{A20} is hydrogen or (C₁-C₈)alkyl. More preferably, R^{A20} is hydrogen or (C₁-C₆)alkyl, still more preferably hydrogen or (C₁-C₄)alkyl, even more preferably hydrogen or (C₁-C₂)alkyl. In preferred embodiments, R^{A20} is hydrogen. R^{A21} and R^{A22} may be each independently selected from the group consisting of hydrogen, (C₁-C₈)alkyl (e.g., methyl, ethyl or propyl), (C₆-C₁₀)aryl (e.g. phenyl), and (C₁-C₈)alkylene(C₆-C₁₀)aryl (e.g. benzyl). Preferably, R^{A21} and R^{A22} are each independently selected from the group consisting of hydrogen or (C₁-C₈)alkyl. More preferably, R^{A21} and R^{A22} are each independently hydrogen or (C₁-C₆)alkyl, still more preferably hydrogen or (C₁-C₄)alkyl, even more preferably hydrogen or (C₁-C₂)alkyl. In preferred embodiments, R^{A21} and R^{A22} are hydrogen. In some embodiments, Y¹ is NR^{A20}, wherein R^{A20} is as defined herein. In some embodiments, Y¹ is O. In some embodiments, Y¹ is S. In some embodiments, Y¹ is CR^{A21}R^{A22}, wherein R^{A20} and R^{A21} are as defined herein.

Preferably, Y¹ is selected from the group consisting of NR^{A20}, O and S, wherein R^{A20} is as defined herein. More preferably, Y¹ is NR^{A20} or O, wherein R^{A20} is as defined herein. More preferably, Y¹ is NH or O. Still more preferably, Y¹ is NR^{A20}, wherein R^{A20} is as defined herein.

In some preferred embodiments, Y¹ is NH.

In some preferred embodiments, Y¹ is O.

In a conjugate of formula (I), any variable, such as, for example, RBM, L, M, X, D, Y¹, E, W, Z and n, may be as defined herein.

Conjugates with a Group Cleavable by Hydrolysis, Esterases, Thioesterases, Proteases or Peptidases

In some preferred embodiments the conjugate has the formula (Ia):

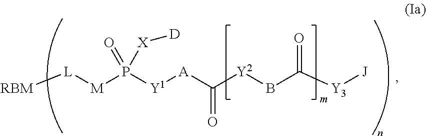

or a pharmaceutically acceptable salt or solvate thereof,
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

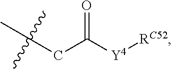

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃—C₃)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl; and
n is an integer ranging from 1 to 20.

Accordingly, in these embodiments, the group

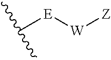

of formula (I)
is:

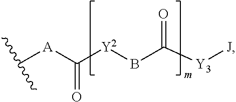

wherein: [symbol]
indicates the attachment to the Y¹; and
A, Y², B, Y³ and J are as defined herein.

In particular, the group A represents the spacer E; the group

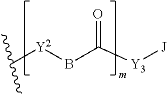

represents the cleavable group Z when m is not 0, or the group

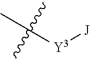

represents the cleavable group Z when m is 0; and the moiety W, after cleavage of the group Z, is a carboxylic acid or carboxylate

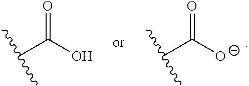

In these embodiments, the group Z can be cleaved, for example, by protease-induced cleavage (Y² or Y³ is NR^{B20}), peptidase-induced cleavage (Y² or Y³ is NR^{B20}), esterase-induced cleavage (Y² or Y³ is O) or thioesterase-induced cleavage (Y² or Y³ is S); when Y² or Y³ is O (i.e., the group

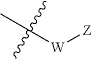

is an ester group), cleavage may occur by hydrolysis inside of a cell also without assistance of an enzyme. The bond between the carbonyl carbon atom adjacent to A and Y², when present (m is not 0), or the bond between the carbonyl carbon atom adjacent to A and Y³ (m is 0) can be cleaved. The group Z can be cleaved at the target site to initiate a reaction that leads to a release of the X-D moiety.

The integer m ranges from 0 to 15. Accordingly, the integer m may be 0, 1, 2, 3, 4, 5, 6, 7, 8, 9, 10, 11, 12, 13, 14 or 15. The integer m may range from 0 to 12. Preferably, the integer m ranges from 0 to 10. More preferably, the integer m ranges from 0 to 8. Still more preferably, the integer m ranges from 0 to 5. Even more preferably, the integer m ranges from 0 to 3. Even more preferably, the integer m is 0 or 1. When the integer m is 0, the groups Y² and B are absent.

In some preferred embodiments, the integer m is 0. Accordingly, the conjugate may have the formula (Ia1):

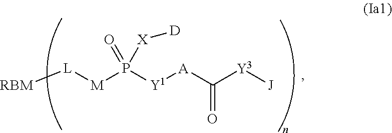

or a pharmaceutically acceptable salt or solvate thereof;
wherein RBM, L, M, X, D, Y¹, A, Y³, J and n are as defined herein.

### Groups Y¹ and A

The group Y¹ is as described herein for any conjugates of formula (I).

Accordingly, Y¹ is selected from the group consisting of NR^{A20}, O, S, or CR^{A21}R^{A22}, wherein R^{A20}, R^{A21} and R^{A22} are as defined herein. R^{A20} may be selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{A20} is hydrogen or (C₁-C₈)alkyl. More preferably, R^{A20} is hydrogen or (C₁-C₆)alkyl, still more preferably hydrogen or (C₁-C₄)alkyl, even more preferably hydrogen or (C₁-C₂)alkyl. In preferred embodiments, R^{A20} is hydrogen. R^{A21} and R^{A22} may be each independently selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{A21} and R^{A22} are each independently selected from the group consisting of hydrogen or (C₁-C₈)alkyl. More preferably, R^{A21} and R^{A22} are each independently hydrogen or (C₁-C₆)alkyl, still more preferably hydrogen or (C₁-C₄)alkyl, even more preferably hydrogen or (C₁-C₂)alkyl. In preferred embodiments, R^{A21} and R^{A22} are hydrogen. In some embodiments, Y¹ is NR^{A20}, wherein R^{A20} is as defined herein. In some embodiments, Y¹ is O. In some embodiments, Y¹ is S. In some embodiments, Y¹ is CR^{A21}R^{A22}, wherein R^{A20} and R^{A21} are as defined herein.

Preferably, Y¹ is selected from the group consisting of NR^{A20}, O and S, wherein R^{A20} is as defined herein. More preferably, Y¹ is NR^{A20} or O, wherein R^{A20} is as defined herein. More preferably, Y¹ is NH or O. Still more preferably, Y¹ is NR^{A20}, wherein R^{A20} is as defined herein.

In some preferred embodiments, Y¹ is NH.

In some preferred embodiments, Y¹ is O.

In some preferred embodiments, A is CR^{A30}R^{A31}, wherein R^{A30} and R^{A31} are as defined herein. Preferably, R^{A30} and R^{A31} are each independently selected from the group consisting of hydrogen, (C₁-C₈)alkyl, (C₆-C₁₀)aryl, and (C₁-C₈)alkylene(C₆-C₁₀)aryl. More preferably, R^{A30} and R^{A31} are each independently selected from the group consisting of hydrogen, (C₁-C₈)alkyl, and (C₁-C₈)alkylene(C₆-C₁₀)aryl. Still more preferably, R^{A30} and R^{A31} are each independently selected from the group consisting of hydrogen and (C₁-C₈)alkyl. Even more preferably, R^{A30} and R^{A31} are each independently selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl. Still even more preferably, R^{A30} and R^{A31} are each independently selected from hydrogen and CH₃. In any one of these embodiments, R^{A30} and R^{A31} may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl. Optionally R^{A30} and R^{A31} can together form a 3 to 8-membered ring. In any one of these embodiments, R^{A30} and R^{A31} may be the same or different. Accordingly, R^{A30} and R^{A31} may be the same. Alternatively, R^{A30} and R^{A31} may be different. In any one of these embodiments, Y¹ may be as defined herein. Preferably, in any one of these embodiments Y¹ may be NR^{A20} or O, wherein R^{A20} is as defined herein. More preferably, in any one of these embodiments Y¹ may be NH or O. Still more preferably, in any one of these embodiments Y¹ may be NR^{A20}, wherein R^{A20} is as defined herein. Even more preferably, in any one of these embodiments Y¹ may be NH.

In some preferred embodiments, A is CR^{A30}R^{A31}, wherein R^{A30} is hydrogen and R^{A31} is as defined herein. Accordingly, R^{A30} may be hydrogen and R^{A31} may be selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl. In one of these embodiments, R^{A31} is not hydrogen. Preferably, R^{A30} is hydrogen and R^{A31} is selected from the group consisting of hydrogen, (C₁-C₈)alkyl, (C₆-C₁₀)aryl, and (C₁-C₈)alkylene(C₆-C₁₀)aryl. In one of these embodiments, R^{A31} is not hydrogen. More preferably, R^{A30} is hydrogen and R^{A31} is selected from the group consisting of (C₁-C₈)alkyl, and (C₁-C₈)alkylene(C₆-C₁₀)aryl. Still more preferably, R^{A30} is hydrogen and R^{A31} is (C₁-C₈)alkyl. Even more preferably, R^{A30} is hydrogen and R^{A31} is selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl. In one of these embodiments, R^{A31} is not hydrogen. Still even more preferably, R^{A30} is hydrogen and R^{A31} is CH₃. In any one of these embodiments, R^{A31} may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl. In any one of these embodiments, Y¹ may be as defined herein. Preferably, in any one of these embodiments Y¹ may be NR^{A20} or O, wherein R^{A20} is as defined herein. More preferably, in any one of these embodiments Y¹ may be NH or O. Still more preferably, in any one of these embodiments Y¹ may be NR^{A20}, wherein R^{A20} is as defined herein. Even more preferably, in any one of these embodiments Y¹ may be NH.

When A is CR^{A30}R^{A31}, the group A in combination with the adjacent carbonyl group and Y¹ may form a group (Aa), which can be depicted as follows:

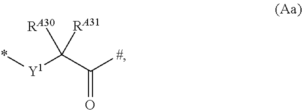

wherein:
the asterisk (*) indicates attachment to the phosphorus;
# indicates attachment to Y², when present (m is not 0), or Y³ (m is 0); and
Y¹, R^{A30} and R^{A31} are as defined herein. R^{A30} and R^{A31} may be the same or different. When R^{A30} and R^{A31} are different, the carbon atom to which R^{A30} and R^{A31} are attached is a chiral center. The carbon atom to which R^{A30} and R^{A31} are attached, when chiral, may be in the (S) or (R) configuration.

Preferably, R^{A30} is hydrogen and R^{A31} is as defined herein. Accordingly, the group (Aa) may be (Ab):

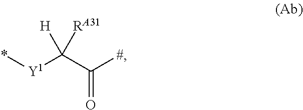

wherein:
the asterisk (*) indicates attachment to the phosphorus;
# indicates attachment to Y², when present (m is not 0), or Y³ (m is 0); and
Y¹ and R^{A31} are as defined herein. When R^{A31} is not hydrogen, the carbon atom to which R^{A31} is attached is a chiral center. The carbon atom to which R^{A31} is attached, when chiral, may be in the (S) or (R) configuration.

More preferably, Y¹ is N^{RA20}. Accordingly the group (Ab) may be (Ac):

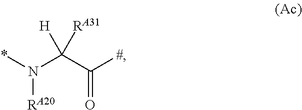

wherein:
the asterisk (*) indicates attachment to the phosphorus;
# indicates attachment to Y², when present (m is not 0), or Y³ (m is 0);
R^{A20} is as defined herein; preferably, R^{A20} is hydrogen; and
R^{A31} is as defined herein. In these embodiments, the group (Ac) represents an amino acid, in particular an alpha-amino acid. When R^{A31} is not hydrogen, the carbon atom to which R^{A31} is attached is a chiral center. The carbon atom to which R^{A31} is attached, when chiral, may be in the (S) or (R) configuration. Preferably, the carbon atom to which R^{A31} is attached, when chiral, is in the (S) configuration. In particular, the amino acid (i.e. the group (Ac)), except for amino acids which are not chiral such as e.g. glycine, may be in the L configuration or the D configuration. Preferably, in any one of the embodiments described herein, the amino acid (i.e. the group (Ac)) is in the L configuration (i.e. in the naturally occurring configuration). In some preferred embodiments, the amino acid is alanine, in particular L-alanine.

As described above, A may be CR^{A30}R^{A31}. However, A is not limited to CR^{A30}R^{A31} but can, in some embodiments, be (C₁-C₈)alkylene. Preferably, in these embodiments A is (C₁-C₆)alkylene. More preferably, A is (C₁-C₄)alkylene. Still more preferably, A is (C₁-C₈)alkylene. Even more preferably, A is (C₂-C₈)alkylene. In some embodiments, A is (C₁-C₂)alkylene. In some embodiments, A is C₁-alkylene (methylene). In any one of the these embodiments the alkylene ((C₁-C₈)alkylene, (C₁-C₆)alkylene, (C₁-C₄)alkylene, (C₁-C₈)alkylene, (C₂-C₈)alkylene, (C₁-C₂)alkylene, or C₁-alkylene (methylene)) may be optionally substituted with one or more substituents each independently selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl.

In some embodiments, A may be

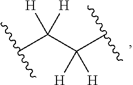 [SMILES: *CC*]

wherein [symbol] indicates attachment to the group Y¹ and the carbonyl carbon atom. Optionally, in the

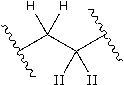 [SMILES: *CC*]

one or more hydrogen atom(s) (in particular, one hydrogen atom) may be replaced with a substituent each independently selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl. In these embodiments, Y¹ may be NR^{A20}, wherein R^{A20} is as defined herein; preferably wherein NR^{A20} is hydrogen. Accordingly, when Y¹ is NR^{A20}, in these embodiments the groups Y¹, A and the adjacent carbonyl group represent a beta-amino acid:

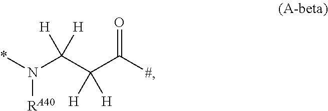

wherein:
the asterisk (*) indicates attachment to the phosphorus;
# indicates attachment to Y², when present (m is not 0), or Y³ (m is 0); and
R^{A20} is as defined herein; preferably R^{A20} is hydrogen;
optionally, in the

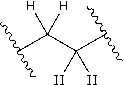 [SMILES: *CC*]

moiety one or more hydrogen atom(s) (in particular, one hydrogen atom) may be replaced with a substituent each independently selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl. In one embodiment, the beta-amino acid may be beta-alanine.

In some embodiments, A may be

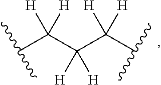 [SMILES: *CCC*]

wherein [symbol] indicates attachment to the group Y¹ and the carbonyl carbon atom. Optionally, in the

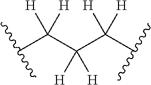 [SMILES: *CCC*]

one or more hydrogen atom(s) (in particular, one hydrogen atom) may be replaced with a substituent each independently selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37}, wherein R^{A36} and R^{A37}, which may be the same or different, are independently selected from (C₁-C₈)alkyl, (C₁-C₈)alkylene(C₆-C₁₀)aryl or (C₆-C₁₀)aryl. In these embodiments, Y¹ may be NR^{A20}, wherein R^{A20} is as defined herein; preferably wherein NR^{A20} is hydrogen. Accordingly, when Y¹ is NR^{A20}, in these embodiments the groups Y¹, A and the adjacent carbonyl group represent a gamma-amino acid:

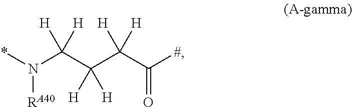

wherein:
the asterisk (*) indicates attachment to the phosphorus;
# indicates attachment to Y², when present (m is not 0), or Y³ (m is 0); and
R^{A20} is as defined herein; preferably R^{A20} is hydrogen;
optionally, in the

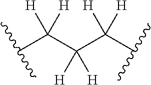 [SMILES: *CCC*]

moiety one or more hydrogen atom(s) (in particular, one hydrogen atom) may be replaced with a substituent each independently selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl.

### Groups Y² and B

The group Y², when present, may be each independently selected from the group consisting of NR^{B20}, O, S, or CR^{B21}R^{B22}, wherein R^{B20}, R^{B21} and R^{B22} are as defined herein. Accordingly, R^{B20} may be selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{B20} is hydrogen or (C₁-C₈)alkyl. More preferably, R^{B20} is hydrogen or (C₁-C₆)alkyl, still more preferably hydrogen or (C₁-C₄)alkyl, even more preferably hydrogen or (C₁-C₂)alkyl. In preferred embodiments, R^{B20} is hydrogen. R^{B21} and R^{B22} may be each independently selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{B21} and R^{B22} are each independently selected from the group consisting of hydrogen or (C₁-C₈)alkyl. More preferably, R^{B21} and R^{B22} are each independently hydrogen or (C₁-C₆)alkyl, still more preferably hydrogen or (C₁-C₄)alkyl, even more preferably hydrogen or (C₁-C₂)alkyl. In preferred embodiments, R^{B21} and R^{B22} are hydrogen. In some embodiments, Y² is NR^{B20}, wherein R^{B20} is as defined herein. In some embodiments, Y² is O. In some embodiments, Y² is S. In some embodiments, Y² is CR^{B21}R^{B22}, wherein R^{B20} and R^{B21} are as defined herein.

Preferably, Y² is each independently selected from the group consisting of NR^{B20}, O and S, wherein R^{B20} is as defined herein. More preferably, Y² is each independently NR^{B20} or O, wherein R^{B20} is as defined herein. More preferably, Y² is each independently NH or O. Still more preferably, each Y² is NR^{B20}, wherein R^{B20} is each independently as defined herein.

In some preferred embodiments, each Y² is NH.

In some embodiments, the group B, when present, is, each independently, CR^{B30}R^{B31}, wherein R^{B30} and R^{B31} are as defined herein. Preferably, R^{B30} and R^{B31} are each independently selected from the group consisting of hydrogen, (C₁-C₈)alkyl, (C₆-C₁₀)aryl, and (C₁-C₈)alkylene(C₆-C₁₀)aryl. More preferably, R^{B30} and R^{B31} are each independently selected from the group consisting of hydrogen, (C₁-C₈)alkyl, and (C₁-C₈)alkylene(C₆-C₁₀)aryl. Still more preferably, R^{B30} and R^{B31} are each independently selected from the group consisting of hydrogen and (C₁-C₈)alkyl. Even more preferably, R^{B30} and R^{B31} are each independently selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl. Still even more preferably, R^{B30} and R^{B31} are each independently selected from hydrogen and CH₃. In any one of these embodiments, R^{B30} and R^{B31} may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl. Optionally R^{B30} and R^{B31} can together form a 3 to 8-membered ring. In any one of these embodiments, R^{B30} and R^{B31} may be the same or different. Accordingly, R^{B30} and R^{B31} may be the same. Alternatively, R^{B30} and R^{B31} may be different. In any one of these embodiments, Y² may be as defined herein. Preferably, in any one of these embodiments Y² may be NR^{B20} or O, wherein R^{B20} is as defined herein. More preferably, in any one of these embodiments Y² may be NH or O. Still more preferably, in any one of these embodiments each Y² may be NR^{B20}, wherein R^{B20} is each independently as defined herein. Even more preferably, in any one of these embodiments Y² may be NH.

In some embodiments, the group B, when present, is CR^{B30}R^{B31}, wherein R^{B30} is hydrogen and R^{B31} is as defined herein. Accordingly, R^{B30} may be hydrogen and R^{B31} may be selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl. In one of these embodiments, R^{B31} is not hydrogen. Preferably, R^{B30} is hydrogen and R^{B31} is selected from the group consisting of hydrogen, (C₁-C₈)alkyl, (C₆-C₁₀)aryl, and (C₁-C₈)alkylene(C₆-C₁₀)aryl. In one of these embodiments, R^{B31} is not hydrogen. More preferably, R^{B30} is hydrogen and R^{B31} is selected from the group consisting of (C₁-C₈)alkyl, and (C₁-C₈)alkylene(C₆-C₁₀)aryl. Still more preferably, R^{B30} is hydrogen and R^{B31} is (C₁-C₈)alkyl. Even more preferably, R^{B30} is hydrogen and R^{B31} is selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl. In one of these embodiments, R^{B31} is not hydrogen. Still even more preferably, R^{B30} is hydrogen and R^{B31} is CH₃. In any one of these embodiments, R^{B31} may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl. In any one of these embodiments, Y² may be as defined herein. Preferably, in any one of these embodiments Y² may be NR^{B20} or O, wherein R^{B20} is as defined herein. More preferably, in any one of these embodiments Y² may be NH or O. Still more preferably, in any one of these embodiments each Y² may be NR^{B20}, wherein R^{B20} is each independently as defined herein. Even more preferably, in any one of these embodiments Y² may be NH.

When B is CR^{B30}R^{B31}, the group B in combination with the adjacent carbonyl group and Y² may form a group (Ba), as follows:

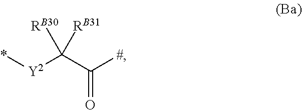

wherein:
the asterisk (*) indicates attachment to the carbonyl carbon atom;
# indicates attachment to Y³; and
Y², R^{B30} and R^{B31} are as defined herein. R^{B30} and R^{B31} may be the same or different. When R^{B30} and R^{B31} are different, the carbon atom to which R^{B30} and R^{B31} are attached is a chiral center. The carbon atom to which R^{B30} and R^{B31} are attached, when chiral, may be in the (S) or (R) configuration.

Preferably, R^{B30} is hydrogen and R^{B31} is as defined herein. Accordingly, the group (Ba) may be (Bb):

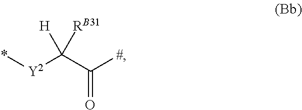

wherein:
the asterisk (*) indicates attachment to the carbonyl carbon atom;
# indicates attachment to Y³; and
Y² and R^{B31} are as defined herein. When R^{B31} is not hydrogen, the carbon atom to which R^{B31} is attached is a chiral center. The carbon atom to which R^{B31} is attached, when chiral, may be in the (S) or (R) configuration.

More preferably, Y² is NR^{B20}. Accordingly the group (Bb) may be (Bc):

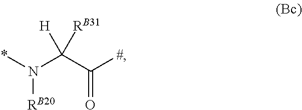

wherein:
the asterisk (*) indicates attachment to the carbonyl carbon atom;
# indicates attachment to Y³; R^{B20} is as defined herein; preferably, R^{B20} is hydrogen; and
R^{B31} is as defined herein. In these embodiments, the group (Bc) represents an amino acid. When R^{B31} is not hydrogen, the carbon atom to which R^{B31} is attached is a chiral center. The carbon atom to which R^{B31} is attached, when chiral, may be in the (S) or (R) configuration. Preferably, the carbon atom to which R^{B31} is attached, when chiral, is in the (S) configuration. In particular, the amino acid (i.e. the group (Bc)), except for amino acids which are not chiral such as e.g. glycine, may be in the L configuration or the D configuration. Preferably, in any one of the embodiments described herein, the amino acid (i.e. the group (Bc)) is in the L configuration (i.e. in the naturally occurring configuration). In some preferred embodiments, the amino acid is alanine, in particular L-alanine.

As described above, B may be CR^{B30}R^{B31}. However, B is not limited to CR^{B30}R^{B31}, but in some embodiments, the group B, when present, may be (C₁-C₈)alkylene. Preferably, in these embodiments B is (C₁-C₆)alkylene. More preferably, B is (C₁-C₄)alkylene. Still more preferably, B is (C₁-C₈)alkylene. Even more preferably, B is (C₂-C₈)alkylene. In some embodiments, B is (C₁-C₂)alkylene. In some embodiments, B is C₁-alkylene (methylene). In any one of the these embodiments the alkylene ((C₁-C₈)alkylene, (C₁-C₆)alkylene, (C₁-C₄)alkylene, (C₁-C₈)alkylene, (C₂-C₈)alkylene, (C₁-C₂)alkylene, or C₁-alkylene (methylene)) may be optionally substituted with one or more substituents each independently selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{A37} wherein R^{B36} and R^{B37}, which may be the same or different, are independently selected from (C₁-C₈)alkyl, (C₁-C₈)alkylene(C₆-C₁₀)aryl or (C₆-C₁₀)aryl.

### Groups Y³ and J

The group Y³ may be selected from the group consisting of O, NR^{C40}, O or S, wherein R^{C40} is as defined herein. Accordingly, R^{C40} may be selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{C40} is hydrogen or (C₁-C₈)alkyl. More preferably, R^{C40} is hydrogen or (C₁-C₆)alkyl, still more preferably hydrogen or (C₁-C₄)alkyl, even more preferably hydrogen or (C₁-C₂)alkyl. In preferred embodiments, R^{C40} is hydrogen. In some embodiments, Y³ is NR^{C40}, wherein R^{C40} is as defined herein. In some embodiments, Y³ is O. In some embodiments, Y³ is S. In some embodiments, Y³ is absent.

Preferably, Y³ is selected from the group consisting of NR^{C40}, O and S, wherein R^{C40} is as defined herein. More preferably, Y³ is NR^{C40} or O, wherein R^{C40} is as defined herein. Still more preferably, Y³ is NH or O.

In some preferred embodiments, Y³ is O.

In some preferred embodiments, Y³ is NR^{C40}, wherein R^{C40} is as defined herein. In some preferred embodiments, Y³ is NH.

In some preferred embodiments, the group J is

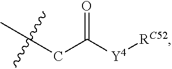

wherein [symbol] , C, Y⁴ and R^{C52} are as defined herein. In any one of these embodiments, Y³ may be as defined herein. Preferably, in any one of these embodiments Y³ may be NR^{C40} or O, wherein R^{C40} is as defined herein. More preferably, in any one of these embodiments Y³ may be NH or O. More preferably, in any one of these embodiments Y³ may be NR^{C40}, wherein R^{C40} is as defined herein. Still more preferably, in any one of these embodiments Y³ may be NH.

According to illustrative but non-limiting examples, when J is

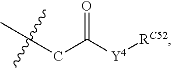

J together with Y³ may represent an amino acid or an ester thereof. As an illustrative but non-limiting example, when Y³ is NH, the group C, in accordance with embodiments which are further described herein below, is CR^{C50}R^{C51} with R^{C50} being hydrogen and R^{C51} being CH₃, Y⁴ is O, and R^{C52} is hydrogen or as further defined herein (e.g., R^{C52} may be (C₁-C₈)alkyl), the group J together with Y³ represents alanine or an ester of alanine. Further to the foregoing example, Y¹ may be NH, A may be CR^{A30}R^{A31} with R^{A30} being hydrogen and R^{A31} being methyl, and the integer m may be 0; accordingly, in such illustrative but nonlimiting example, the group

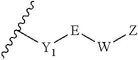

in formula (I), or the group

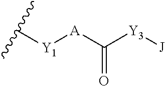

in formula (Ia), represents a di-alanyl moiety having the structure:

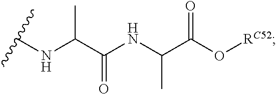

R^{C52} is as defined herein, for example, R^{C52} may be (C₁-C₈)alkyl or hydrogen.

In some preferred embodiments, the group C is CR^{C50}CR^{C51}. Accordingly, in some preferred embodiments, the group J is

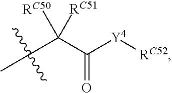

wherein R^{C50}, R^{C51}, Y⁴, R^{C52} and [symbol] are as defined herein. Preferably, R^{C50} and R^{C51} are each independently selected from the group consisting of hydrogen, (C₁-C₈)alkyl, (C₅-C₁₀)aryl, and (C₁-C₈)alkylene(C₅-C₁₀)aryl. More preferably, R^{C50} and R^{C51} are each independently selected from the group consisting of hydrogen, (C₁-C₈)alkyl, and (C₁-C₈)alkylene(C₆-C₁₀)aryl. Still more preferably, R^{C50} and R^{C51} are each independently selected from the group consisting of hydrogen and (C₁-C₈)alkyl. Even more preferably, R^{C50} and R^{C51} are each independently selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl. Still even more preferably, R^{C50} and R^{C51} are each independently selected from the group consisting of hydrogen and CH₃. In any one of these embodiments, R^{C50} and R^{C51} may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl. Optionally R^{C50} and R^{C51} can together form a 3 to 8-membered ring. In any one of these embodiments, R^{C50} and R^{C51} may be the same or different. Accordingly, R^{C50} and R^{C51} may be the same. Alternatively, R^{C50} and R^{C51} may be different. In any one of these embodiments, Y³ may be as defined herein. Preferably, in any one of these embodiments Y³ may be NR^{C40} or O, wherein R^{C40} is as defined herein. More preferably, in any one of these embodiments Y³ may be NH or O. More preferably, in any one of these embodiments Y³ may be NR^{C40}, wherein R^{C40} is as defined herein. Still more preferably, in any one of these embodiments Y³ may be NH.

In some preferred embodiments, R^{C50} is hydrogen and R^{C51} is selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl. In one of these embodiments, R^{C}s¹ is not hydrogen. More preferably, R^{C50} is hydrogen and R^{C}s¹ is selected from the group consisting of hydrogen, (C₁-C₈)alkyl, (C₆-C₁₀)aryl, and (C₁-C₈)alkylene(C₆-C₁₀)aryl. In one of these embodiments, R^{C}s¹ is not hydrogen. Still more preferably, R^{C50} is hydrogen and R^{C}s¹ is selected from the group consisting of (C₁-C₈)alkyl, and (C₁-C₈)alkylene(C₆-C₁₀)aryl. Even more preferably, R^{C50} is hydrogen and R^{C51} is (C₁-C₈)alkyl. Still even more preferably, R^{C50} is hydrogen and R^{C51} is selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl. In one of these embodiments, R^{C51} is not hydrogen. Still even more preferably, R^{C50} is hydrogen and R^{C51} is CH₃. In any one of these embodiments, R^{C51} may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl. In any one of these embodiments, Y³ may be as defined herein. Preferably, in any one of these embodiments Y³ may be NR^{C40} or O, wherein R^{C40} is as defined herein. More preferably, in any one of these embodiments Y³ may be NH or O. More preferably, in any one of these embodiments Y³ may be NR^{C4}°, wherein R^{C4}° is as defined herein. Still more preferably, in any one of these embodiments Y³ may be NH.

Y⁴ is as defined herein. Accordingly, Y⁴ may be selected from the group consisting of O, NR^{C53}, S and CR^{C54}R^{C55}. R^{C53} may be selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{C53} is hydrogen or (C₁-C₈)alkyl. More preferably, R^{C53} is hydrogen or (C₁-C₆)alkyl, still more preferably hydrogen or (C₁-C₄)alkyl, even more preferably hydrogen or (C₁-C₂)alkyl. In preferred embodiments, R^{C53} is hydrogen. R^{C54} and R^{C55} may be each independently selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{C54} and R^{C55} are each independently selected from the group consisting of hydrogen or (C₁-C₈)alkyl. More preferably, R^{C54} and R^{C55} are each independently hydrogen or (C₁-C₆)alkyl, still more preferably hydrogen or (C₁-C₄)alkyl, even more preferably hydrogen or (C₁-C₂)alkyl. In preferred embodiments, R^{C54} and R^{C55} are hydrogen. In some embodiments, Y⁴ is O. In some embodiments, Y⁴ is NR^{C53}, wherein R^{C53} is as defined herein. In some embodiments, Y⁴ is S. In some embodiments, Y⁴ is CR^{C54}R^{C55}, wherein R^{C54} and R^{C55} are as defined herein. In some embodiments, Y⁴ is absent.

Preferably, Y⁴ is O or NR^{C53}, wherein R^{C53} is as defined herein. More preferably, Y⁴ is O or NH. In some preferred embodiments, Y⁴ is O.

R^{C52} is as defined herein. Preferably, R^{C52} is selected from the group consisting of hydrogen, (C₁-C₈)alkyl, (C₆-C₁₀)aryl, and (C₁-C₈)alkylene(C₆-C₁₀)aryl. More preferably, R^{C52} is selected from the group consisting of hydrogen, (C₁-C₈)alkyl, and (C₁-C₈)alkylene(C₆-C₁₀)aryl. Still more preferably, R^{C52} is selected from the group consisting of hydrogen and (C₁-C₈)alkyl. Even more preferably, R^{C52} is selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl. Still even more preferably, R^{C52} is selected from the group consisting of hydrogen, CH(CH₃)₂ and C(CH₃)₃. Still even more preferably, R^{C52} is hydrogen. In preferred embodiments, R^{C52} is (C₁-C₈)alkyl. In some embodiments, R^{C52} is CH(CH₃)₂. In some embodiments, R^{C52} is C(CH₃)₃. In any one of these embodiments, R^{C52} may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57}, wherein R^{C56} and R^{C57}, which may be the same or different, are independently selected from (C₁-C₈)alkyl, (C₁-C₈)alkylene(C₆-C₁₀)aryl or (C₆-C₁₀)aryl. In any one of these embodiments, Y³ may be as defined herein. Preferably, in any one of these embodiments Y³ may be NR^{C40} or O, wherein R^{C40} is as defined herein. More preferably, in any one of these embodiments Y³ may be NH or O. More preferably, in any one of these embodiments Y³ may be NR^{C40}, wherein R^{C40} is as defined herein. Still more preferably, in any one of these embodiments Y³ may be NH. In any one of these embodiments, Y⁴ may be as defined herein. Preferably, in any one of these embodiments, Y⁴ may be O (oxygen).

When the group J is

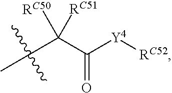

the group J in combination with the group Y³ may form a group (Ja), as follows:

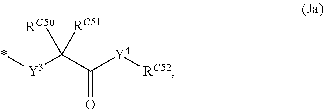

wherein:
the asterisk (*) indicates attachment to the carbonyl carbon atom; and
Y³, R^{C50}, R^{C51}, Y⁴ and R^{C52} are as defined herein. R^{C50} and R^{C51} may be the same or different. When R^{C50} and R^{C51} are different, the carbon atom to which R^{C50} and R^{C51} are attached is a chiral center. The carbon atom to which R^{C50} and R^{C51} are attached, when chiral, may be in the (S) or (R) configuration.

Preferably, R^{C50} is hydrogen and R^{C51} is as defined herein. Accordingly, the group (Ja) may be (Jb):

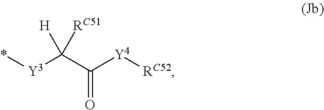

wherein:
the asterisk (*) indicates attachment to the carbonyl carbon atom; and
Y³, R^{C51}, Y⁴ and R^{C52} are as defined herein. When R^{C51} is not hydrogen, the carbon atom to which R^{C51} is attached is a chiral center. The carbon atom to which R^{C51} is attached, when chiral, may be in the (S) or (R) configuration.

More preferably, Y³ is NR^{C40}. Accordingly the group (Jb) may be (Jc):

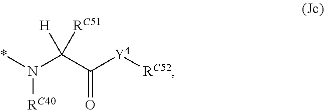

wherein:
the asterisk (*) indicates attachment to the carbonyl carbon atom;
R^{C51}, Y⁴ and R^{C52} are as defined herein; and
R^{C40} is as defined herein; preferably, R^{C40} is hydrogen. In these embodiments, the group (Jc) represents an amino acid. When R^{C51} is not hydrogen, the carbon atom to which R^{C51} is attached is a chiral center. The carbon atom to which R^{C51} is attached, when chiral, may be in the (S) or (R) configuration. Preferably, the carbon atom to which R^{C51} is attached, when chiral, is in the (S) configuration. In particular, the amino acid (i.e. the group (Jc)), except for amino acids which are not chiral such as e.g. glycine, may be in the L configuration or the D configuration. Preferably, in any one of the embodiments described herein, the amino acid (i.e. the group (Jc)) is in the L configuration (i.e. in the naturally occurring configuration). In some preferred embodiments, the amino acid is alanine, in particular L-alanine.

In some preferred embodiments, A is CR^{A30}R^{A31} and J is

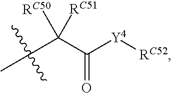

wherein R^{A30}, R^{A31}, R^{C50}, R^{C51}, Y⁴, R^{C52} [symbol] and are as defined herein. In any one of these embodiments the integer m may be 0.

In some more preferred embodiments, A is CR^{A30}R^{A31} and J is

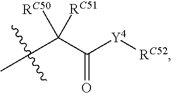

wherein R^{A30}, R^{A31}, R^{C50}, R^{C51}, R^{C52} and [symbol] are as defined herein Y¹ is NR^{A20}, Y³ is NR^{C40}, and Y⁴ is O, wherein R^{A20} and R^{C40} are as defined herein. More preferably, in any one of these embodiments Y¹ is NH, Y³ is NH and Y⁴ is O. In any one of these embodiments the integer m may be 0.

In some also preferred embodiments, A is CR^{A30}R^{A31} and J is

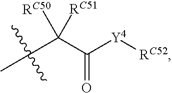

wherein R^{A30} is hydrogen, R^{A31} is CH₃, R^{C50} is hydrogen, R^{C51} is CH₃, R^{C52} is hydrogen, [symbol] is as defined herein, Y¹ is NR^{A20}, Y³ is NR^{C40}, and Y⁴ is O, wherein R^{A20} and R^{C40} are as defined herein. Preferably, in any one of these embodiments Y¹ is NR^{A20}, Y³ is NR^{C40}, and Y⁴ is O, wherein R^{A20} and R^{C40} are as defined herein. More preferably, in any one of these embodiments Y¹ is NH, Y³ is NH and Y⁴ is O. In any one of these embodiments the integer m may be 0.

As described above, J may be

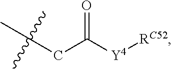

wherein the group C is CR^{C50}R^{C51}. However, in the structure of

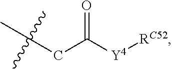

C is not limited to CR^{C50}R^{C51}, but can, in some embodiments, be (C₁-C₈)alkylene. Preferably, in these embodiments, C is (C₁-C₆)alkylene. More preferably, C is (C₁-C₄)alkylene. Still more preferably, C is (C₁-C₈)alkylene. Even more preferably, C is (C₂-C₈)alkylene. In some embodiments, C is (C₁-C₂)alkylene. In some embodiments, C is C₁-alkylene (methylene). In any one of the these embodiments the alkylene ((C₁-C₈)alkylene, (C₁-C₆)alkylene, (C₁-C₄)alkylene, (C₁-C₈)alkylene, (C₂-C₈)alkylene, (C₁-C₂)alkylene, or C₁-alkylene (methylene)) may be optionally substituted with one or more substituents each independently selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl.

As described above, J may be

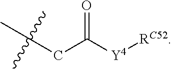

However, J is not limited to the structure of

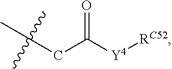

but can, in alternative embodiments, be selected from the group consisting of (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl. More preferably, J is selected from the group consisting of (C₁-C₈)alkyl, (C₆-C₁₀)aryl, and (C₁-C₈)alkylene(C₆-C₁₀)aryl. Still more preferably, J is selected from the group consisting of (C₁-C₈)alkyl and (C₁-C₈)alkylene(C₆-C₁₀)aryl. Even more preferably, J is (C₁-C₈)alkyl. Still even more preferably, J is selected from the group consisting of CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl. Still even more preferably, J is CH(CH₃)₂ or C(CH₃)₃. In some embodiments, J is CH(CH₃)₂. In some embodiments, J is C(CH₃)₃. In any one of these embodiments, J may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47}, wherein R^{C46} and R^{C47}, which may be the same or different, are independently selected from (C₁-C₈)alkyl, (C₁-C₈)alkylene(C₆-C₁₀)aryl or (C₆-C₁₀)aryl. In any one of these embodiments the integer m may be 0. In any one of these embodiments, Y³ may be as defined herein. Preferably, in any one of these embodiments Y³ may be O or NR^{C40}, wherein R^{C40} is as defined herein. More preferably, in any one of these embodiments, Y³ may be O. In any one of these embodiments, A may be as defined herein. Preferably, in any one of these embodiments A may be CR^{A30}R^{A31} wherein R^{A30} and R^{A31} are as defined herein. In any one of these embodiments, Y¹ and Y³ may be as defined herein. Preferably, in any one of these embodiments Y¹ may be NR^{A20} and Y³ may be 0, wherein R^{A20} is as defined herein. More preferably, in any one of these embodiments Y¹ may be NH and Y³ may be O. In any one of these embodiments the integer m may be 0.

As illustrative non-limiting example, when J is as defined in the foregoing paragraph, Y³ is O, Y¹ is NH and the integer m is 0, the group J together with Y³, A and Y¹ represents an ester of an amino acid. In particular, according to an illustrative but non-limiting example, when J is as defined in the foregoing paragraph, Y³ is O, the integer m is 0, A is CR^{A30}R^{A31} with R^{A30} being hydrogen and R^{A31} being CH₃, and Y¹ is NH, the group

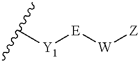

in formula (I), or the group

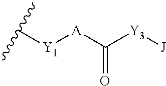

in formula (Ia), represents an alanyl moiety (also denoted as mono-alanyl moiety) having the structure:

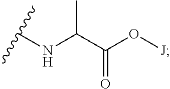

J is as defined in the foregoing paragraph, for example, J may be (C₁-C₈)alkyl.

In some preferred embodiments, R^{A30} is hydrogen, R^{A31} is CH₃, Y¹ is NH, and Y³ is O. Preferably, in any one of these embodiments J may be CH(CH₃)₂ or C(CH₃)₃. Accordingly, in some of these embodiments J may be CH(CH₃)₂. In some of these embodiments J may be C(CH₃)₃. In any one of these embodiments the integer m may be 0.

In a conjugate of formula (Ia) or (Ia1), any variable, such as, for example, RBM, L, M, X, D, Y¹, A, Y², B, m, Y³, J and n, may be as defined herein.

Conjugates with a Cleavable Sugar Moiety

In some embodiments the conjugate has the formula (Ib)

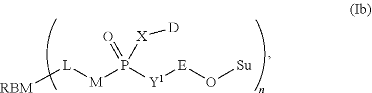

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Su is a sugar moiety which is bound to the oxygen atom (O) via a cleavable bond; and
wherein the oxygen, after cleavage of the sugar moiety Su, is capable of forming a ring, preferably a four- to seven-membered ring, more preferably a five- or six-membered ring, together with the spacer E, Y¹ and the phosphorus; and
n is an integer ranging from 1 to 20.

Accordingly, in these embodiments, the group

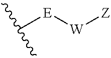

in formula (I) is:

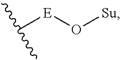

wherein E is a spacer as described herein;
Su is a sugar moiety which is bound to the oxygen atom (O) via a cleavable bond; and
wherein the oxygen, after cleavage of the sugar moiety Su, is capable of forming a ring, preferably a four- to seven-membered ring, more preferably a five- or six-membered ring, together with the spacer E, Y¹ and the phosphorus.

In these embodiments, the sugar moiety Su represents the cleavable group Z, and the moiety W, after cleavage of the group Z, is a hydroxy group

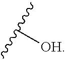 [SMILES: *O]

The sugar moiety Su, i.e. the cleavable group Z, can be cleaved, for example, by glycodisase-induced cleavage. In particular, the bond between the Sugar moiety Su and the oxygen (O) can be cleaved. The group Z, i.e. the sugar moiety Su, can be cleaved at the target site to initiate a reaction that leads to a release of the X-D moiety. The bond between the sugar moiety Su and the oxygen (O) may be a glycosidic bond. The term “glycosidic bond”, in general refers to a bond between the carbon atom of the hemiacetal moiety (in other words, the anomeric carbon atom) of the sugar moiety Su and the oxygen (O). The sugar moiety is not particularly limited and can be any suitable glycoside, including glycosides that are modified with suitable protecting groups at the hydroxyl functionalities. Suitable protecting groups are generally known and include, for example, acyl esters such as acetyl ester, lactic acid esters, ethers, sulfate groups or phosphate moieties. The protecting groups at each hydroxy function of the sugar moiety may be the same or different from each other. The sugar moiety may be selected, for example, from the group consisting of glucuronic acid, galactose, glucose, arabinose, mannose-6-phosphate, fucose, rhamnose, gulose, allose, 6-deoxy-glucose, lactose, maltose, cellobiose, gentiobiose, maltotriose, GlcNAc, GalNAc and maltohexaose with and without protecting groups at the hydroxyl functionalities.

Without wishing to be bound by theory, a possible mechanism for drug release is depicted for an exemplary compound of formula (Ib) in the following scheme.

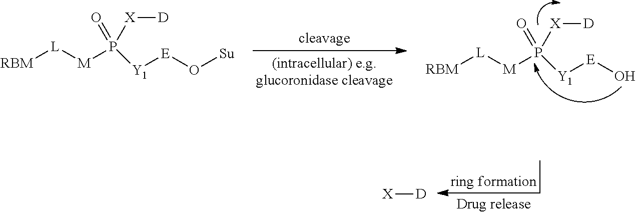

In some generally preferred embodiments, the sugar moiety is a sugar moiety that is modified with suitable protecting groups at the hydroxyl functionalities. A sugar moiety that is modified with suitable protecting groups at the hydroxyl functionalities may also be referred to herein as “protected sugar moiety”.

Preferably, each hydroxy function of the sugar moiety Su is protected with an acetyl ester.

In some embodiments, the sugar moiety is glucuronic acid:

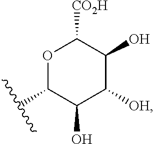 [SMILES: *[C@@H]1O[C@H](C(=O)O)[C@@H](O)[C@H](O)[C@H]1O]

wherein [symbol] indicates the position of the oxygen atom (O). In particular, the glucuronic acid may be recognized, and the glycosidic bond to the oxygen atom (O) may be cleaved by a glucuronidase, such as, for example, a beta-glucuronidase.

In some embodiments, the sugar moiety is a glucuronic acid with suitable protecting groups at the hydroxyl functionalities. Preferably, the protected glucuronic acid has the following structure:

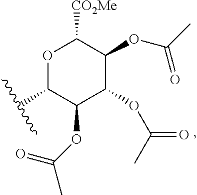 [SMILES: *[C@@H]1O[C@H](C(=O)OC)[C@@H](OC(C)=O)[C@H](OC(C)=O)[C@H]1OC(C)=O]

wherein [symbol] indicates the position of the oxygen atom (O). In particular, the glucuronic acid may be recognized after removal of the protecting groups to liberate the glucuronic acid and the glycosidic bond to the oxygen atom (O) may be cleaved by a glucuronidase, such as, for example, a beta-glucuronidase.

In a conjugate of formula (Ib), the spacer E may have the following structure A:

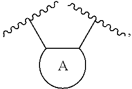

which is an optionally substituted four- to seven-membered, preferably five- or six-membered, carbocyclic or heterocyclic ring;
wherein [symbol] indicate the positions of the oxygen atom and Y¹. Preferably, in any one of these embodiments the attachment points of A to the oxygen atom and Y¹ are two adjacent atoms of the ring. Preferably,

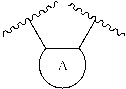

may have the following structure:

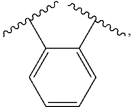 [SMILES: *c1ccccc1*]

wherein [symbol] indicate the positions of the oxygen atom and Y¹.

In a conjugate of formula (Ib), Y¹ may be as defined herein. Preferably, Y¹ is NR^{A20} or O, wherein R^{A20} is as defined herein. More preferably, Y¹ is NH or O. Still more preferably, Y¹ is NH.

Preferably, the conjugate of formula (Ib) has formula (Ib1):

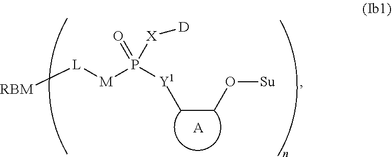

or a pharmaceutically acceptable salt or solvate thereof;
wherein RBM, L, M, X, D, Y¹,

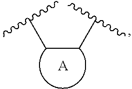

[symbol]

Su and n are as defined herein.

In a conjugate of formula (Ib) or (Ib1) any variable, such as, for example, RBM, L, M, X, D, Y¹,

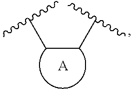

[symbol]

E, Su and n, may be as defined herein.
Conjugates with a Cleavable Disulfide Moiety

In some embodiments the conjugate has the formula (Ic)

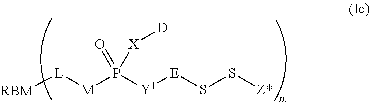

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z* is an optionally substituted aliphatic residue or an optionally substituted aromatic residue; wherein the sulfur bound to the spacer E, after cleavage of the disulfide bond, is capable of forming a ring, preferably a four- to seven-membered ring, more preferably a five- or six-membered ring, together with the spacer E, Y¹ and the phosphorus; and
n is an integer ranging from 1 to 20.

Accordingly, in these embodiments, the group

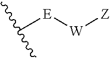

in formula (I) is:

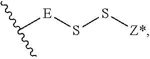

wherein E is a spacer as described herein;
Z* is an optionally substituted aliphatic residue or an optionally substituted aromatic residue; and
wherein the sulfur bound to the spacer E, after cleavage of the disulfide bond, is capable of forming a ring, preferably a four- to seven-membered ring, more preferably a five- or six-membered ring, together with the spacer E, Y¹ and the phosphorus.

In these embodiments, the moiety

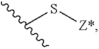

represents the cleavable group Z, and the moiety W, after cleavage of the group Z, is a thiol group

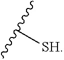 [SMILES: *S]

The group

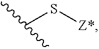

i.e. the cleavable group Z, can be cleaved, for example, by enzymatic reduction. In particular, the disulfide bond can be cleaved by enzymatic reduction. The group Z, i.e. the group

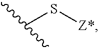

can be cleaved at the target site to initiate a reaction that leads to a release of the X-D moiety.

Without wishing to be bound by theory, a possible mechanism for drug release is depicted for an exemplary compound of formula (Ic) in the following scheme.

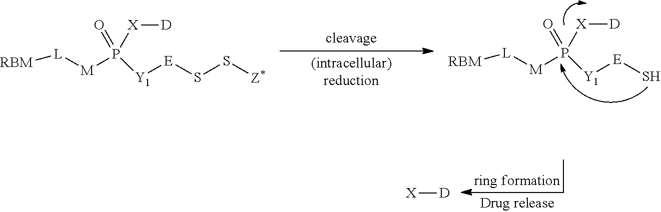

In a conjugate of formula (Ic) the spacer E may be

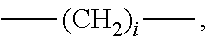

which can be optionally substituted; and
wherein i is an integer ranging from 1 to 4. Preferably, i is 2, 3 or 4. More preferably, i is 2 or 3. Still more preferably, i is 2. Optionally, one or more hydrogen atom(s) (in particular, one hydrogen atom) of the —(CH₂)ᵢ— may be replaced with a substituent each independently selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl.

In a conjugate of formula (Ic), Z* is an optionally substituted aliphatic residue or an optionally substituted aromatic residue. In some embodiments, Z* may be optionally substituted (C₁-C₈)alkyl. In some embodiments, Z* is methyl, ethyl, propyl (such as, e.g., iso-propyl) or butyl (such as, e.g., tert-butyl).

Preferably, a conjugate of formula (Ic) has formula (Ic1):

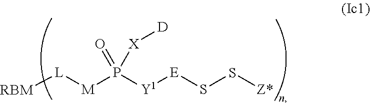

or a pharmaceutically acceptable salt or solvate thereof;
wherein:
RBM, L, M, X, D, Y¹, Z* and n are as defined herein;
E is

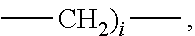

which can be optionally substituted; and wherein i is an integer ranging from 1 to 4; preferably 2, 3 or 4; more preferably 2 or 3; still more preferably 2.

In a conjugate of formula (Ic) or (Ic1), any variable, such as, for example, RBM, L, M, X, D, Y¹, E, i, Z* and n, may be as defined herein.

Conjugates with a Cleavable Acetal Moiety

In some embodiments the conjugate has the formula (Id):

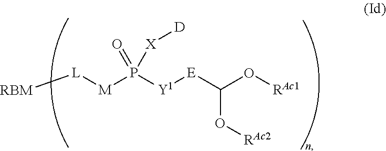

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
R^{Ac1} and R^{Ac2} are each independently an optionally substituted aliphatic residue or an optionally substituted aromatic residue; and
n is an integer ranging from 1 to 20.

Accordingly, in these embodiments, the group

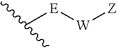

in formula (I) is:

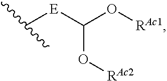

wherein E is a spacer as described herein. Without wishing to be bound by theory, a possible mechanism for drug release is depicted for an exemplary compound of formula (Id) in the following scheme.

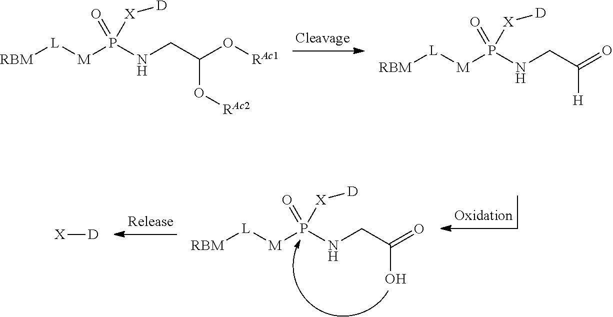

Accordingly, on a mechanistic level it can be assumed that the acetal moiety can be cleaved, e.g. by hydrolysis, to give an aldehyde. Oxidation of the aldehyde by an aldehyde oxidase may form a carboxylic acid or carboxylate moiety, which represents the group W, and which can attack the phosphorus atom and effect release of the drug moiety (X-D), for example, in accordance with a mechanism as explained herein above. The group

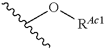

and/or

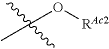

can be considered as the cleavable group Z. Accordingly, conjugates of formula (Id) are illustrative examples that, in addition to cleavage of the group Z, one or more further steps may occur, in particular after cleavage of the group Z, to provide a group W (in the present example, an oxidation step), which group W is then capable of forming a ring (preferably, a four- to seven-membered ring, more preferably a five- or six-membered ring) together with the spacer E, Y¹ and the phosphorus. This example also shows that an acetal group can be used as a masking group for a carbonyl group, such as an aldehyde or ketone. This shows that it is also within the scope of the present invention to use an aldehyde or a ketone compound (moiety) in order to provide the group W.

In a conjugate of formula (Id), R^{Ac1} and R^{Ac2} are each independently an optionally substituted aliphatic residue or an optionally substituted aromatic residue. In some embodiments, R^{Ac1} and R^{Ac2} are each independently optionally substituted (C₁-C₈)alkyl. In some embodiments, R^{Ac1} and R^{Ac2} are each independently methyl, ethyl, propyl (such as, e.g., iso-propyl) or butyl (such as, e.g., tert-butyl). In any one of these embodiments R^{Ac1} and R^{Ac2} may be same or different; preferably, R^{Ac1} and R^{Ac2} are the same. Optionally, R^{Ac1} and R^{Ac2} can together with the oxygen atoms and the carbon atom form a 3- to 8-membered ring.

In a conjugate of formula (Id), the spacer E may be any spacer as defined herein. In some embodiments, the spacer E is a group A as defined herein, such as, for example, with regard to conjugates of formula (Ia). Accordingly, in some embodiments the conjugate of formula (Id) has formula (Id1):

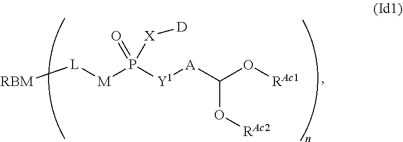

or a pharmaceutically acceptable salt or solvate thereof;
wherein:
RBM, L, M, X, D, Y¹, R^{Ac1}, R^{Ac2} and n are as defined herein;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; and
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring.

In a conjugate of formula (Id) or (Id1) any variable, such as, for example, RBM, L, M, X, D, Y¹, E, A, R^{Ac1}, R^{Ac2} and n, may be as defined herein. In particular, A may be as defined herein with regard to conjugates of formula (Ia).

Conjugates with a Cleavable Amide Moiety

In some embodiments the conjugate has the formula (Ie)

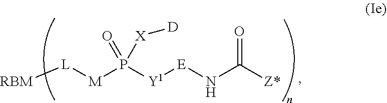

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z* is an optionally substituted aliphatic residue or an optionally substituted aromatic residue; wherein the nitrogen atom bound to the spacer E, after cleavage of the acetyl bond, is capable of forming a ring, preferably a four- to seven-membered ring, more preferably a five-or six-membered ring, together with the spacer E, Y¹ and the phosphorus; and
n is an integer ranging from 1 to 20.

Accordingly, in these embodiments, the group

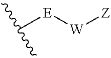

in formula (I) is:

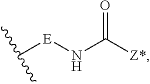

wherein E is a spacer as described herein;
Z* is an optionally substituted aliphatic residue or an optionally substituted aromatic residue; and
wherein the nitrogen atom bound to the spacer E, after cleavage of the acetyl bond, is capable of forming a ring, preferably a four- to seven-membered ring, more preferably a five-or six-membered ring, together with the spacer E, Y¹ and the phosphorus.

In these embodiments, the moiety

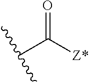

represents the cleavable group Z, and the moiety W, after cleavage of the group Z, is an amino group

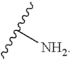 [SMILES: *N]

The group

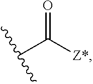

i.e. the cleavable group Z, can be cleaved, for example, by peptidase or protease to give an amine

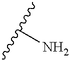 [SMILES: *N]

The group Z, i.e. the group

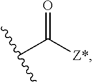

can be cleaved at the target site to initiate a reaction that leads to a release of the X-D moiety.

Without wishing to be bound by theory, a possible mechanism for drug release is depicted for an exemplary compound of formula (If) in the following scheme.

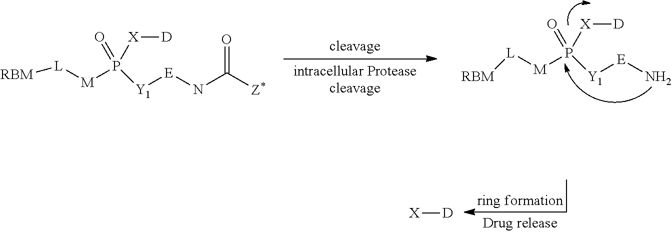

In a conjugate of formula (Ie) the spacer E may be any spacer E as described herein. In some embodiments of formula (Ie), the spacer E may be,

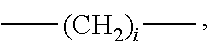

which can be optionally substituted; and
wherein i is an integer ranging from 1 to 4. Preferably, i is 2, 3 or 4. More preferably, i is 2 or 3. Still more preferably, i is 2. Optionally, one or more hydrogen atom(s) (in particular, one hydrogen atom) of the —(CH₂)ᵢ— may be replaced with a substituent each independently selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl.

In a conjugate of formula (Ie), Z* is an optionally substituted aliphatic residue or an optionally substituted aromatic residue. In some embodiments, Z* may be optionally substituted (C₁-C₈)alkyl. In some embodiments, Z* is methyl, ethyl, propyl (such as, e.g., iso-propyl) or butyl (such as, e.g., tert-butyl).

Preferably, a conjugate of formula (Ie) has formula (Ie1):

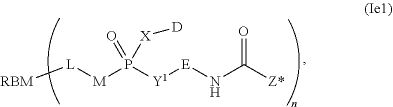

or a pharmaceutically acceptable salt or solvate thereof;
wherein:
RBM, L, M, X, D, Y¹, Z* and n are as defined herein;
E is

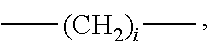

which can be optionally substituted; and wherein i is an integer ranging from 1 to 4; preferably 2, 3 or 4; more preferably 2 or 3; still more preferably 2.

In a conjugate of formula (Ie) or (Ie1), any variable, such as, for example, RBM, L, M, X, D, Y¹, E, i, Z* and n, may be as defined herein.

Conjugates with a Cleavable Ester Moiety

In some embodiments the conjugate has the formula (If)

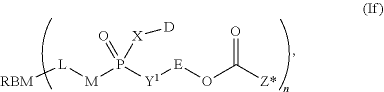

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z* is an optionally substituted aliphatic residue or an optionally substituted aromatic residue;
wherein the oxygen atom bound to the spacer E, after cleavage of the acetyl bond, is capable of forming a ring, preferably a four- to seven-membered ring, more preferably a five-or six-membered ring, together with the spacer E, Y¹ and the phosphorus; and
n is an integer ranging from 1 to 20.

Accordingly, in these embodiments, the group

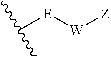

in formula (I) is:

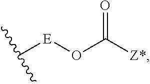

wherein E is a spacer as described herein;
Z* is an optionally substituted aliphatic residue or an optionally substituted aromatic residue; and
wherein the nitrogen atom bound to the spacer E, after cleavage of the acetyl bond, is capable of forming a ring, preferably a four- to seven-membered ring, more preferably a five-or six-membered ring, together with the spacer E, Y¹ and the phosphorus.

In these embodiments, the moiety

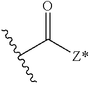

represents the cleavable group Z, and the moiety W, after cleavage of the group Z, is an alcohol. The group

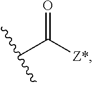

i.e. the cleavable group Z, can be cleaved, for example, by peptidase or protease to give an amine

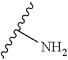 [SMILES: *N]

The group Z, i.e. the group

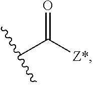

can be cleaved at the target site to initiate a reaction that leads to a release of the X-D moiety.

Without wishing to be bound by theory, a possible mechanism for drug release is depicted for an exemplary compound of formula (Ie) in the following scheme.

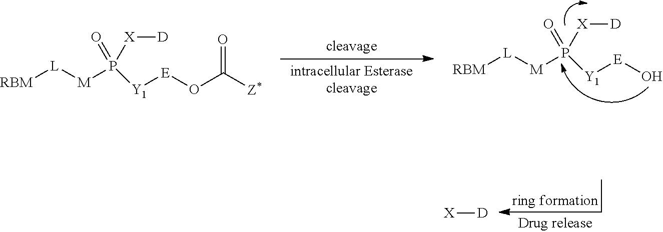

In a conjugate of formula (Ie) the spacer E may be any spacer E as described herein. In some embodiments of formula (Ie), the spacer E may be,

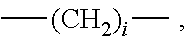

which can be optionally substituted; and
wherein i is an integer ranging from 1 to 4. Preferably, i is 2, 3 or 4. More preferably, i is 2 or 3. Still more preferably, i is 2. Optionally, one or more hydrogen atom(s) (in particular, one hydrogen atom) of the —(CH₂)ᵢ— may be replaced with a substituent each independently selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl.

In a conjugate of formula (Ie), Z* is an optionally substituted aliphatic residue or an optionally substituted aromatic residue. In some embodiments, Z* may be optionally substituted (C₁-C₈)alkyl. In some embodiments, Z* is methyl, ethyl, propyl (such as, e.g., iso-propyl) or butyl (such as, e.g., tert-butyl).

Preferably, a conjugate of formula (If) has formula (If1):

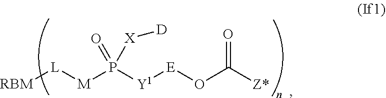

or a pharmaceutically acceptable salt or solvate thereof;
wherein:
RBM, L, M, X, D, Y¹, Z* and n are as defined herein;
E is

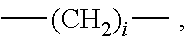

which can be optionally substituted; and wherein i is an integer ranging from 1 to 4; preferably 2, 3 or 4; more preferably 2 or 3; still more preferably 2.

In a conjugate of formula (Ie) or (Ie1), any variable, such as, for example, RBM, L, M, X, D, Y¹, E, i, Z* and n, may be as defined herein.

### Integer n

The integer n may range from 1 to 20. Accordingly, the integer n may be 1, 2, 3, 4, 5, 6, 7, 8, 9, 10, 11, 12, 13, 14, 15, 16, 17, 18, 19 or 20. In the conjugates described herein, the integer n denotes the ratio of drug moieties (D) to the receptor binding molecule (RBM).

In some embodiments, the integer n ranges from 1 to 14. Accordingly, the integer n may be 1, 2, 3, 4, 5, 6, 7, 8, 9, 10, 11, 12, 13 or 14.

In some embodiments, the integer n ranges from 1 to 14. Preferably, the integer n ranges from 2 to 14. Still more preferably the integer n ranges from 3 to 14. Even more preferably the integer n ranges from 4 to 14. Even more preferably the integer n ranges from 5 to 12. Even more preferably, the integer n ranges from 6 to 12. Even more preferably from 7 to 10. Even more preferably, the integer n is 7 or 8. Even more preferably, the integer n is 8.

In some embodiments, the integer n ranges from 1 to 14. Preferably, the integer n ranges from 1 to 12. Still more preferably, the integer n ranges from 2 to 10. Even more preferably, the integer n ranges from 2 to 8. Even more preferably, the integer n ranges from 2 to 6. Even more preferably, the integer n ranges from 3 to 5. Even more preferably, the integer n is 4.

### Receptor Binding Molecule (RBM)

RBM is a receptor binding molecule. The term “receptor binding molecule” in general refers to any molecule which is capable to bind to a receptor. As illustrative but non-limiting example, the receptor, to which a receptor binding molecule may bind, may be expressed on a cell surface. As illustrative but non-limiting example, the cell which expresses the receptor, may be a cancer cell. A person skilled in the art knows to select a suitable receptor binding molecule.

The receptor may be a tumor associated surface antigen. Accordingly, the receptor binding molecule may be capable to specifically bind to a tumor associated surface antigen. The term “tumour associated surface antigen” as used herein in general refers to an antigen that is or can be presented on a surface that is located on or within tumour cells. These antigens can be presented on the cell surface with an extracellular part, which is often combined with a transmembrane and cytoplasmic part of the molecule. These antigens can in some embodiments be presented only by tumour cells and not by normal, i.e. non-tumour cells. Tumour antigens can be exclusively expressed on tumour cells or may represent a tumour specific mutation compared to non-tumour cells. In such an embodiment a respective antigen may be referred to as a tumour-specific antigen. Some antigens are presented by both tumour cells and non-tumour cells, which may be referred to as tumour-associated antigens. These tumour-associated antigens can be overexpressed on tumour cells when compared to non-tumour cells or are accessible for antibody binding in tumour cells due to the less compact structure of the tumour tissue compared to non-tumour tissue. In some embodiments the tumour associated surface antigen is located on the vasculature of a tumour. Illustrative but non-limiting examples of a tumour associated surface antigen include Trop2 or Her2. Tumor associated surface antigens, are known to a person skilled in the art. In particular, those which have been found useful for the development of ADCs are described, e.g., in the review article of Criscitello et al., “Antibody-drug conjugates in solid tumors: a look into novel targets”, Journal of Hematology and Oncology, (2021) 14:20 (https://doi.org/10.1186/s13045-021-01035-z).

In some embodiments, the receptor binding molecule may be selected from the group consisting of an antibody, an antibody fragment, a proteinaceous binding molecule with antibody-like binding properties, an aptamer, and a small molecule. In some embodiments, the receptor binding molecule is an aptamer. In some embodiments, the receptor binding molecule is a small molecule.

Preferably, the receptor binding molecule is an antibody. More preferably, the antibody is selected from the group consisting of a monoclonal antibody, a chimeric antibody, a humanized antibody, a human antibody, and a single domain antibody, such as a camelid or shark single domain antibody. Still more preferably, the antibody is a monoclonal antibody. Preferably, the antibody is capable to a specifically bind to a tumour associated surface antigen. In some embodiments, the antibody may be Sacituzumab. In some embodiments, the antibody may be Trastuzumab.

The receptor binding molecule may be an antibody fragment. Preferably, the antibody fragment is a divalent antibody fragment. More preferably, the divalent antibody fragment is selected from the group consisting of a (Fab)₂′-fragment, a divalent single-chain Fv fragment, a dual affinity re-targeting (DART) antibody, and a diabody. Alternatively, preferably the antibody fragment is a monovalent antibody fragment. More preferably the monovalent antibody fragment is selected from the group consisting of a Fab fragment, a Fv fragment, and a single-chain Fv fragment (scFv). It is also possible that the monovalent antibody fragment is a fragment of a single domain camelid or shark single domain antibody. Preferably, the antibody fragment is capable to specifically bind to a tumour associated surface antigen.

The receptor binding molecule may be a proteinaceous binding molecule with antibody-like binding properties. Examples of proteinaceous binding molecules with antibody-like binding properties that can be used as receptor binding molecule include, but are not limited to, an aptamer, a mutein based on a polypeptide of the lipocalin family, a glubody, a protein based on the ankyrin scaffold, a protein based on the crystalline scaffold, an adnectin, an avimer, a EGF-like domain, a Kringle-domain, a fibronectin type I domain, a fibronectin type II domain, a fibronectin type Ill domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or a an immunoglobulin-like domain (for example, domain antibodies or camel heavy chain antibodies), a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C₂ domain, “Kappabodies” (III. et al. “Design and construction of a hybrid immunoglobulin domain with properties of both heavy and light chain variable regions” Protein Eng 10:949-57 (1997)), “Minibodies” (Martin et al. “The affinity-selection of a minibody polypeptide inhibitor of human interleukin-6” EMBO J 13:5303-9 (1994)), “Janusins” (Traunecker et al. “Bispecific single chain molecules (Janusins) target cytotoxic lymphocytes on HIV infected cells” EMBO J 10:3655-3659 (1991) and Traunecker et al. “Janusin: new molecular design for bispecific reagents” Int J Cancer Suppl 7:51-52 (1992), a nanobody, a adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein, an avimer (Silverman, Lu Q, Bakker A, To W, Duguay A, Alba BM, Smith R, Rivas A, Li P, Le H, Whitehorn E, Moore KW, Swimmer C, Perlroth V, Vogt M, Kolkman J, Stemmer WP 2005, Nat Biotech, Dec;23(12):1556-61, E-Publication in Nat Biotech. 2005 November 20 edition); as well as multivalent avimer proteins evolved by exon shuffling of a family of human receptor domains as also described in Silverman J, Lu Q, Bakker A, To W, Duguay A, Alba BM, Smith R, Rivas A, Li P, Le H, Whitehorn E, Moore KW, Swimmer C, Perlroth V, Vogt M, Kolkman J, Stemmer WP, Nat Biotech, Dec;23(12):1556-61, E-Publication in Nat. Biotechnology. 2005 November 20 edition. Preferably, the proteinaceous binding molecule with antibody-like binding properties is selected from the group consisting of a mutein based on a polypeptide of the lipocalin family, a glubody, a protein based on the ankyrin scaffold, a protein based on the crystalline scaffold, an adnectin, an avimer, a DARPin, and an affibody. Preferably, the proteinaceous binding molecule with antibody-like binding properties is capable to specifically bind to a tumour associated surface antigen.

### Group X and Drug Moiety (D)

The group X serves to connect the drug moiety (D) to the phosphorus atom. The group X may be provided by the drug moiety. In particular, the group X may form part of the drug before attachment to the phosphorus. Illustrative but non-limiting examples that the group X is provided by the drug moiety are represented by the drugs SN-38 and DXD, as depicted in the following:

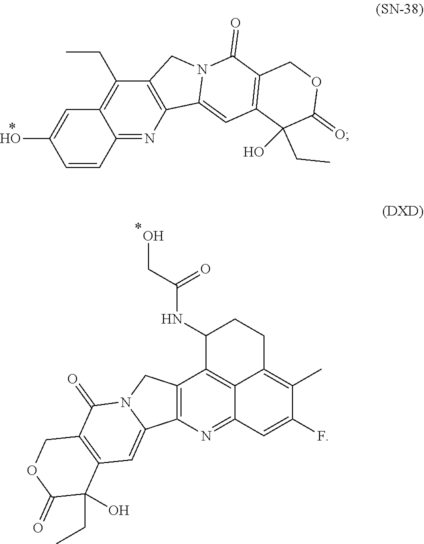 [SMILES: CCc1c2c(nc3ccc(O)cc13)-c1cc3c(c(=O)n1C2)COC(=O)C3(O)CC ; CCC1(O)C(=O)OCc2c1cc1n(c2=O)Cc2c-1nc1cc(F)c(C)c3c1c2C(NC(=O)CO)CC3]

These drug moieties may be bound to the phosphorus via the hydroxy group marked with an asterisk (*). Accordingly, in these embodiments, the oxygen (O) of the hydroxy group marked with an asterisk represents the group X. A person skilled in the art readily recognizes that the hydrogen atom connected to the X group (i.e., for example, the hydrogen atom connected to the oxygen atom marked with an asterisk in the above structures) can be present when the drug is not attached to the phosphorus; the hydrogen atom can thus be present before attachment of the drug moiety to the phosphorus, or after release of the drug from the conjugate. A person skilled in the art knows how to modify a drug so that is comprises a group X suitable for attachment to the phosphorus, in case this should be necessary.

In any one of the embodiments described herein, the group X may be O, S, or NR^{X10}. R^{X10} may be hydrogen; or an optionally substituted aliphatic residue or an optionally substituted aromatic residue. In particular, in any one of the embodiments described herein, R^{X10} may be selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{X10} is hydrogen or (C₁-C₈)alkyl. More preferably, R^{X10} is hydrogen or (C₁-C₆)alkyl, still more preferably hydrogen or (C₁-C₄)alkyl, even more preferably hydrogen or (C₁-C₂)alkyl. In preferred embodiments, R^{X10} is hydrogen. R^{X10} may be optionally substituted. In some embodiments, X may be selected from the group consisting of O, S and NR^{X1}O, wherein R^{X10} is as defined herein. In some embodiments X is O. In some embodiments X is S. In some embodiments X is NR^{X10}, wherein R^{X10} is as defined herein, preferably R^{X10} is hydrogen. In some embodiments, X is O or NR^{X10}, wherein R^{X10} is as defined herein. In some embodiments, X is O or S.

Preferably, the group X is O (oxygen).

Further preferred, the group X is NH.

The moiety X-D may be derived from an aliphatic alcohol. The moiety X-D may be derived from an aromatic alcohol. In these embodiments, X is O. The term “aromatic alcohol” by itself or part of a larger structure in particular refers to an aromatic ring system substituted with the hydroxyl functional group —OH. Thus, the term “aromatic alcohol” refers to any aryl, heteroaryl, arylene and heteroarylene moiety as described herein having a hydroxyl functional group bonded to an aromatic carbon of its aromatic ring system. The aromatic alcohol may be part of a larger moiety, in particular of a drug moiety (D), as when its aromatic ring system is a substituent of this moiety, or may be embedded into the larger moiety, in particular the drug moiety (D), by ring fusion, and may be optionally substituted with moieties as described herein including one or more other hydroxyl substitutents. A phenolic alcohol is an aromatic alcohol having a phenol group as the aromatic ring. The term “aliphatic alcohol” by itself or part of a larger structure in particular refers to a moiety having a non-aromatic carbon bonded to the hydroxyl functional group —OH. The hydroxy-bearing carbon may be unsubstituted (i.e., methyl alcohol) or may have one, two or three optionally substituted branched or unbranched alkyl substituents to define a primary alcohol, or a secondary or tertiary aliphatic alcohol within a linear or cyclic structure. When forming part of a larger structure, in particular of a drug moiety (D), the alcohol may be a substituent of this structure by bonding through the hydroxy bearing carbon, through a carbon of an alkyl or other moiety as described herein to this hydroxyl-bearing carbon or through a substituent of this alkyl or other moiety. The term “aliphatic alcohol” also contemplates a non-aromatic cyclic structure (i.e., carbocycles and heterocarbocycles, optionally substituted) in which a hydroxy functional group is bonded to a non-aromatic carbon of its cyclic ring system. The terms “derived from an aromatic alcohol” or “derived from an aliphatic alcohol” in particular mean that the hydrogen atom of the hydroxy group (—OH) is replaced by the phosphorus of a conjugate or compound described herein, so that the oxygen (i.e. the group X) of the hydroxy group (—OH) is bound to the phosphorus of a conjugate or compound described herein.

In preferred embodiments, X is O and the drug moiety (D) is an optionally substituted aliphatic residue. In preferred embodiments, X is O and the drug moiety (D) is an optionally substituted aromatic residue.

The present disclosure provides conjugates, such as e.g. antibody drug conjugates, comprising a drug moiety D. The term “drug moiety” or “payload”, both of which can be used interchangeably, as used herein refers to a chemical or biochemical moiety that is conjugated to a receptor binding molecule (RBM), such as e.g. an antibody or antigen binding fragment. In this regard, it is again referred to the conjugate of formula (I) described herein. The receptor binding molecule (RBM) can be conjugated to several identical or different drug moieties using any methods described herein or known in the art. In some embodiments, the drug moiety may be a molecule which has a cytotoxic effect on mammalian cells, may lead to apoptosis, and/or may have a modulating effect on malignant cells.

The drug moiety D is not particularly limited and may be any suitable drug moiety. In some preferred embodiments, the drug moiety is, a Mitotic Spindle-Inhibitor such as (−)-Epipodophyllotoxin, a Dehydrogenase A-Inhibitor such as (R)-GNE-140, a Kinase-Inhibitor such as (S)-3-Hydroxy Midostaurin and (R)-3-Hydroxy Midostaurin, a BET-Inhibitor such as ABBV-744, a Estrogene Receptor Agonist such as Acolbifene, a Wee1-Inhibitor such as Adavosertib, a HSP90-Inhibitor such as Alvespimycin, a Kinase-Inhibitor such as ARS-1620, a FGFR-Inhibitor such as ASP5878, a MCT1-Inhibitor such as AZD3965, a mTOR-Inhibitor such as AZD-8055, a Kinase-Inhibitor such as Belizatinib, a HIF-2a inhibitor such as Belzutifan, a BCL-Inhibitor such as BM-1197, a VEGFR-Inhibitor such as Brivanib, a STAT3-Inhibitor such as C188, a anti tumor such as CB1151, a Kinase-Inhibitor such as Dasatinib, a EGFR-Inhibitor such as DBPR112, a CDK-Inhibitor such as Dinaciclib, a TRPC4 and TRCP5 Channel Activator such as Englerin A, a PRMT-Inhibitor such as EPZ015666, a Topoisomerase-Inhibitor such as Etoposide, a mTOR-Inhibitor such as Everolimus, a Methyltransferase-Inhibitor such as EZM 2302, a CDK-Inhibitor such as Fadraciclib, a USP7-Inhibitor such as FT671, a Estrogene Receptor Agonist such as Fulvestrant, a Estrogene Receptor Agonist such as Fulvestrant, a HSP90-Inhibitor such as Geldanamycin, a Estrogene Receptor Agonist such as GNE-274, a Kinase-Inhibitor such as GNE-493, a PRMT-Inhibitor such as GSK3326595, a Kinase-Inhibitor such as Hypothemycin, a CDK-Inhibitor such as IIIM-290, a DNA alkylator such as Illudin S, a Kinase-Inhibitor such as Ilorasertib, a Kinase-Inhibitor such as Larotrectinib, a Kinase-Inhibitor such as Larotrectinib, a IGF-1-Inhibitor such as Linsitinib, a PRMT-Inhibitor such as LLY-283, a HSP90-Inhibitor such as Luminespib, a FGFR-Inhibitor such as LY2874455, a Kinase-Inhibitor such as Mirdametinib, a Kinase-Inhibitor such as MRTX1133, a Kinase-Inhibitor such as MRTX1133, a Kinase-Inhibitor such as Ningetinib, DNA minor groove binder such as Lurbinectidin or Trabectidin, a HSP90-Inhibitor such as NMS-E973, a Ribonucleotide Reductase-Inhibitor such as NSAH, a PLK1-Inhibitor such as Onvansertib, a mTOR-Inhibitor such as Palomid 529, a Kinase-Inhibitor such as PD166326, a NEDD8-Inhibitor such as Pevonedistat, a Kinase-Inhibitor such as PF-04217903, a Kinase-Inhibitor such as PF-06843195, a HSP90-Inhibitor such as PI-103, a Methyltransferase-Inhibitor such as Pinometostat, a Topoisomerase-Inhibitor such as PNU-159682, a Topoisomerase-Inhibitor such as Podofilox, a HDAC-Inhibitor such as QTX125, a mTOR-Inhibitor such as Rapamycin, a Tankyrase-Inhibitor such as RK-287107, a Kinase-Inhibitor such as RO4987655, a Kinase-Inhibitor such as RP-3500, a BCL-Inhibitor such as S55746, a BCL-Inhibitor such as S65487, a EGFR-Inhibitor such as SDZ281-977, a Kinase-Inhibitor such as SU14813, a Kinase-Inhibitor such as TC-A 2317, a Kinase-Inhibitor such as Teleocidin A1, a Ribonucleotide Reductase-Inhibitor such as Tezacitabine, a Kinase-Inhibitor such as TG 100572, a Inhibitor of RNA splicing such as Thailanstatin A, a Kinase-Inhibitor such as UNC5293, a Kinase-Inhibitor such as UNC5293, a HSP90-Inhibitor such as VER-50589, a eIF4A-Inhibitor such as Zotatifin and analogues or prodrugs thereof.

In further embodiments, the drug moiety is an anti-cancer agent. Accordingly, in any one of the compounds described herein as the drug moiety may be selected from the group consisting of camptothecin compounds, TOPK inhibitors (such as e.g. OTS-964), CDK inhibitors (such as e.g. Ganetespib or Roniciclib), bromodomain inhibitors (such as e.g. Brirabresib), HSP70 inhibitors (such as e.g. Triptolide), HSP90 inhibitors (such as e.g. SNX-2112), ribonucleotide reductase inhibitors (such as e.g. Gemcitabine), Aurora B kinase inhibitors (such as e.g. Barasertib), auristatins (such as e.g. monomethyl auristatin E (MMAE) or monomethyl auristatin F (MMAF)), maytansinoids, calicheamycins, tubulysins, amatoxins, dolastatins, pyrrolobenzodiazepine dimers, indolino-benzodiazepine dimers, radioisotopes, therapeutic proteins and peptides (or fragments thereof), KSP inhibitors, eIF4E inhibitors (such as e.g. ON-013100), nicotinamide phosphoribosyltransferase (Nampt) inhibitors (such as e.g. Nampt-IN-1), dihydroorotate dehydrogenase (DHODH) inhibitors (such as e.g. Bay-2402234 or DHODH-IN-16), taxanes (such as e.g. Paclitaxel, albumin-bound Paclitaxel (nab-Paclitaxel), Docetaxel, Cabazitaxel or Abraxan), and analogues or prodrugs thereof.

Preferably, the drug moiety is a camptothecin compound. The term “camptothecin compound” includes camptothecin itself and analogues of camptothecin. Camptothecin is a topoisomerase poison, which was discovered in 1966 by M. E. Wall and M. C. Wani in systematic screening of natural products for anticancer drugs. Camptothecin was isolated from the bark and stem of Camptotheca acuminata (Camptotheca, Happy tree), a tree native to China used as a cancer treatment in Traditional Chinese Medicine. Camptothecin has the following structure:

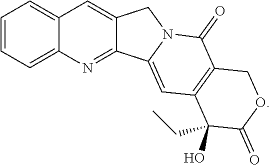 [SMILES: CC[C@@]1(O)C(=O)OCc2c1cc1n(c2=O)Cc2cc3ccccc3nc2-1]

The term “campthothecin compound” also comprises camptothecin analogoues. In this regard, the term “camptothecin compound” denotes any compound which comprises the structure of camptothecin:

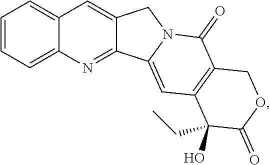 [SMILES: CC[C@@]1(O)C(=O)OCc2c1cc1n(c2=O)Cc2cc3ccccc3nc2-1]

and which may be optionally substituted. The optional substituents may include, as illustrative non-limiting examples, (C₁-C₁₀)alkyl, (C₃-C₈)carbocyclo, (C₃-C₈)heterocyclo, aryl, an amino group, a hydroxy group, a carbonyl group, an amide group, an ester group, a carbamate group, a carbonate group and/or a silyl group. The camptothecin compound may have one or more functional group(s) which are capable to form a bond to the linker L. A person skilled in the art will readily select a suitable camptothecin compound having a desired biological activity. Camptothecin analogues have been approved and are used in cancer chemotherapy today, such as e.g. topotecan, irinotecan, or belotecan.

The following camptothecin analogues are also envisioned by the term camptothecin compound:

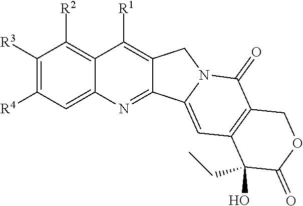

| Analogue | R¹ | R² | R³ | R⁴ |
|---|---|---|---|---|
| Topotecan | —H | 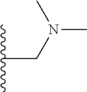 [SMILES: *CN(C)C] | —OH | —H |
| Irinotecan (CPT-11) | 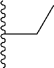 [SMILES: *CC] | —H | 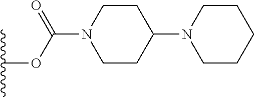 [SMILES: *OC(=O)N1CCC(N2CCCCC2)CC1] | —H |
| Silatecan (DB-67, AR-67) | 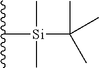 [SMILES: *[Si](C)(C)C(C)(C)C] | —H | —OH | —H |
| Cositecan (BNP-1350) | 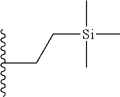 [SMILES: *CC[Si](C)(C)C] | —H | —H | —H |
| Exatecan | 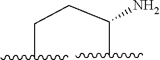 [SMILES: *CC[C@@H](*)N] | | —CH₃ | —F |
| Lurtotecan | 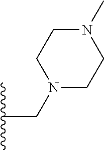 [SMILES: *CN1CCN(C)CC1] | —H | 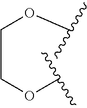 [SMILES: *OCCO*] | |
| Gimatecan (ST1481) | 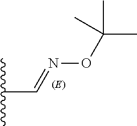 [SMILES: */C=N/OC(C)(C)C] | —H | —H | —H |
| Belotecan (CKD-602) | 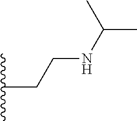 [SMILES: *CCNC(C)C] | —H | —H | —H |
| Rubitecan | —H | 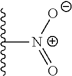 [SMILES: *[N+](=O)[O-]] | —H | —H |

Further camptothecin analogues, which may be used as camptothecin compound, are described in WO 2019/236954 and EP 0 495 432, which are hereby incorporated by reference.

In some embodiments, the camptothecin compound is selected from the group consisting of DXD, SN38, exatecan, camptothecin, topotecan, irinotecan, belotecan, lurtotecan, rubitecan, silatecan, cositecan and gimatecan. Preferably, the camptothecin compound is DXD or SN38.

Preferably, the drug moiety D is DXD. DXD has the following structure:

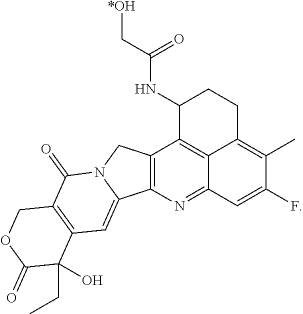

In some preferred embodiments, DXD has the following structure:

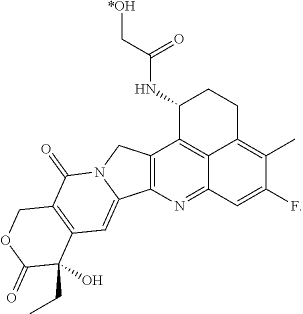

Preferably, in any one of these embodiments the DXD may be bound to the phosphorus atom via the hydroxy group marked with an asterisk (*); accordingly, in these embodiments the oxygen atom of the hydroxy group marked with the asterisk represents the group X.

Preferably, the drug moiety is SN38. SN38 has the following structure:

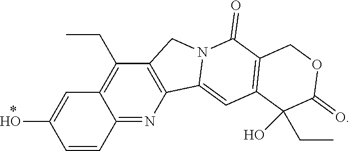 [SMILES: CCc1c2c(nc3ccc(O)cc13)-c1cc3c(c(=O)n1C2)COC(=O)C3(O)CC]

In some preferred embodiments, SN38 has the following structure:

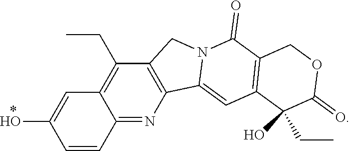 [SMILES: CCc1c2c(nc3ccc(O)cc13)-c1cc3c(c(=O)n1C2)COC(=O)[C@]3(O)CC]

Preferably, in any one of these embodiments the SN38 may be bound to the phosphorus atom via the hydroxy group marked with an asterisk (*). Accordingly, in these embodiments the oxygen atom of the hydroxy group marked with the asterisk represents the group X.

Preferably, the drug moiety is Exatecan. Exatecan has the following structure:

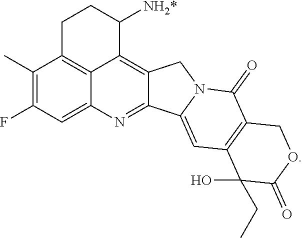

In some preferred embodiments, Exatecan has the following structure:

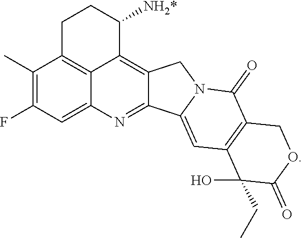

Preferably, in any one of these embodiments the Exatecan may be bound to the phosphorus atom via the amino group marked with an asterisk (*). Accordingly, in these embodiments the nitrogen atom of the amino group marked with the asterisk represents the group X.

Preferably, the drug moiety is a TOPK inhibitor. The TOPK inhibitor may be OTS-964. In some preferred embodiments, the drug moiety is OTS-964. OTS-964 has the following structure:

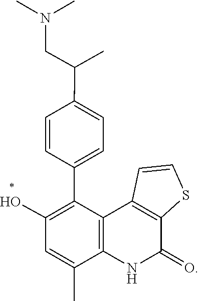 [SMILES: Cc1cc(O)c(-c2ccc(C(C)CN(C)C)cc2)c2c1[nH]c(=O)c1sccc12]

In some more preferred embodiments, OTS-964 has the following structure:

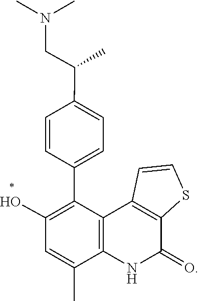 [SMILES: Cc1cc(O)c(-c2ccc([C@@H](C)CN(C)C)cc2)c2c1[nH]c(=O)c1sccc12]

Preferably, in any one of these embodiments the OTS-964 may be bound to the phosphorus atom via the hydroxy group marked with an asterisk (*). Accordingly, in these embodiments the oxygen atom of the hydroxy group marked with the asterisk represents the group X.

Preferably, the drug moiety is a CDK inhibitor. The CDK inhibitor may be ganetespib or Roniciclib. In some preferred embodiments, the drug moiety is ganetespib. Ganetespib has the following structure:

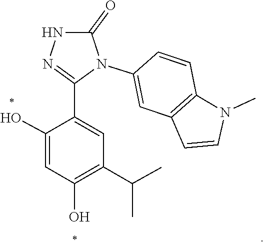 [SMILES: CC(C)c1cc(-c2n[nH]c(=O)n2-c2ccc3c(ccn3C)c2)c(O)cc1O]

Preferably, in any one of these embodiments the ganetespib may be bound to the phosphorus atom via any of the hydroxy groups marked with an asterisk (*). Accordingly, in these embodiments the oxygen atom of the hydroxy group marked with the asterisk represents the group X. In some embodiments, it is also possible that any conjugate or compound described herein comprising ganetespib is a mixture of the two isomers resulting from binding via the hydroxy groups.

In some preferred embodiments, the drug moiety is Roniciclib. Roniciclib has the following structure:

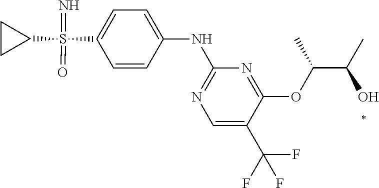 [SMILES: C[C@@H](O)[C@@H](C)Oc1nc(Nc2ccc([S@](=N)(=O)C3CC3)cc2)ncc1C(F)(F)F]

Preferably, in any one of these embodiments the Roniciclib may be bound to the phosphorus atom via the hydroxy group marked with an asterisk (*). Accordingly, in these embodiments the oxygen atom of the hydroxy group marked with the asterisk represents the group X.

Preferably, the drug moiety is a bromodomain inhibitor. The bromodomain inhibitor may be birabresib. In some preferred embodiments, the drug moiety is birapresib. Birabresib has the following structure:

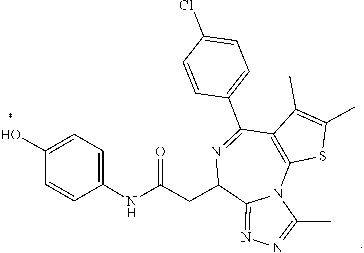 [SMILES: Cc1sc2c(c1C)C(c1ccc(Cl)cc1)=NC(CC(=O)Nc1ccc(O)cc1)c1nnc(C)n1-2]

In some preferred embodiments, birabresib has the following structure:

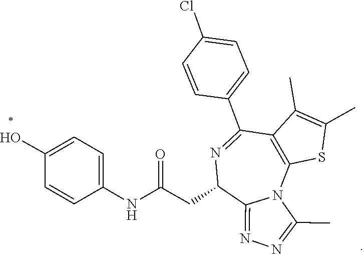 [SMILES: Cc1sc2c(c1C)C(c1ccc(Cl)cc1)=N[C@@H](CC(=O)Nc1ccc(O)cc1)c1nnc(C)n1-2]

Preferably, in any one of these embodiments the birabresib may be bound to the phosphorus atom via the hydroxy group marked with an asterisk (*). Accordingly, in these embodiments the oxygen atom of the hydroxy group marked with the asterisk represents the group X.

Preferably, the drug moiety is a HSP70 inhibitor. The HSP700 inhibitor may be Triptolide. In some preferred embodiments, the drug moiety is Triptolide. Triptolide has the following structure:

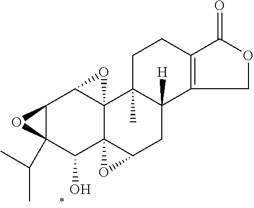

Preferably, in any one of these embodiments the Triptolide may be bound to the phosphorus atom via the hydroxy group marked with an asterisk (*). Accordingly, in these embodiments the oxygen atom of the hydroxy group marked with the asterisk represents the group X.

Preferably, the drug moiety is a HSP90 inhibitor. The HSP90 inhibitor may be SNX-2112. In some preferred embodiments, the drug moiety is SNX-2112. SNX-2112 has the following structure:

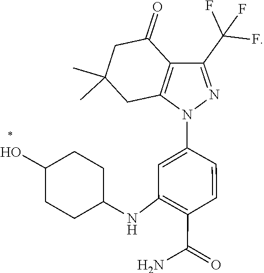 [SMILES: CC1(C)CC(=O)c2c(C(F)(F)F)nn(-c3ccc(C(N)=O)c(NC4CCC(O)CC4)c3)c2C1]

In some preferred embodiments, SNX-2112 has the following structure:

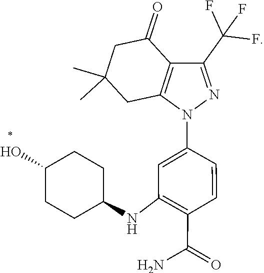 [SMILES: CC1(C)CC(=O)c2c(C(F)(F)F)nn(-c3ccc(C(N)=O)c(N[C@H]4CC[C@H](O)CC4)c3)c2C1]

Preferably, in any one of these embodiments the SNX-2112 may be bound to the phosphorus atom via the hydroxy group marked with an asterisk (*). Accordingly, in these embodiments the oxygen atom of the hydroxy group marked with the asterisk represents the group X.

Preferably, the drug moiety is a ribonucleotide reductase inhibitor. The ribonucleotide reductase inhibitor may be gemcitabine. In some preferred embodiments, the drug moiety is gemcitabine. Gemcitabine has the following structure:

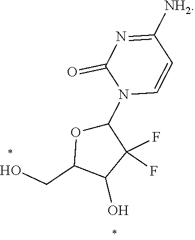 [SMILES: Nc1ccn(C2OC(CO)C(O)C2(F)F)c(=O)n1]

In some preferred embodiments, gemcitabine has the following structure:

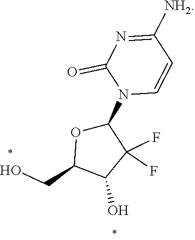 [SMILES: Nc1ccn([C@@H]2O[C@H](CO)[C@@H](O)C2(F)F)c(=O)n1]

Preferably, in any one of these embodiments the gemcitabine may be bound to the phosphorus atom via any of the hydroxy groups marked with an asterisk (*). Accordingly, in these embodiments the oxygen atom of the hydroxy group marked with the asterisk represents the group X. In some embodiments, it is also possible that any conjugate or compound described herein comprising gemcitabine is a mixture of the two isomers resulting from binding via the hydroxy groups.

Preferably, the drug moiety is an Aurora B kinase inhibitor. The Aurora B kinase inhibitor may be barasertib. In some preferred embodiments, the drug moiety is barasertib. Barasertib has the following structure:

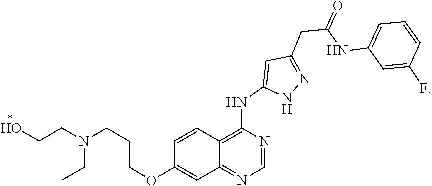 [SMILES: CCN(CCO)CCCOc1ccc2c(Nc3cc(CC(=O)Nc4cccc(F)c4)n[nH]3)ncnc2c1]

Preferably, in any one of these embodiments the barasertib may be bound to the phosphorus atom via the hydroxy group marked with an asterisk (*). Accordingly, in these embodiments the oxygen atom of the hydroxy group marked with the asterisk represents the group X.

Preferably, the drug moiety D is an auristatin. In some embodiments, the auristatin is monomethyl auristatin E (MMAE). In some embodiments, the auristatin is monomethyl auristatin F (MMAF).

Monomethyl auristatin E (MMAE) is represented by the following structural formula:

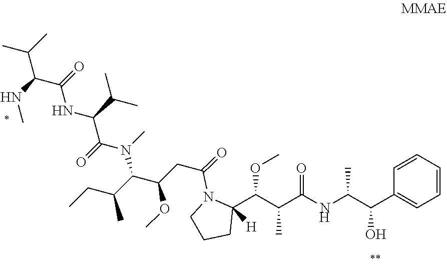

MMAE may be bound to the phosphorus, e.g., via the N terminus indicated with an asterisk (*); accordingly, in these embodiments the N-methyl group marked with an asterisk (*) represents the group X. In alternative embodiments, MMAE may be bound to the phosphorus via the hydroxy group marked with two asterisks (**); in these embodiments the oxygen atom of the hydroxy group marked with two asterisks (**) represents the group X; optionally, when the MMAE is bound to the phosphorus via the hydroxy group marked with two asterisks (**), the N terminus indicated with an asterisk (*) may be protected with a suitable protecting group, such as e.g. with a tert-butyloxycarbonyl group (BOC group).

Monomethyl auristatin F (MMAF) is represented by the following structural formula:

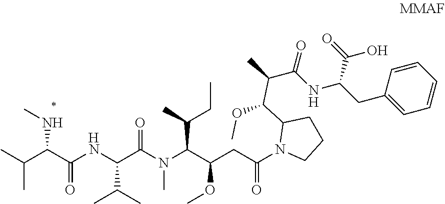

MMAF may be bound to the phosphorus, e.g., via the N terminus indicated with an asterisk (*); accordingly, in these embodiments the N-methyl group marked with an asterisk (*) represents the group X.

These molecules noncompetitively inhibit binding of vincristine to tubulin (at a location known as the vinca/peptide region) but have been shown to bind to the RZX/MAY region.

In some embodiments the drug moiety is a maytansinoid drug moiety, including those having the structure:

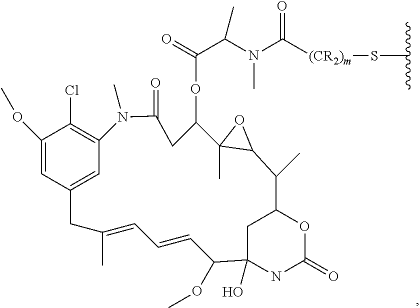

where the wavy line indicates the covalent attachment of the sulfur atom of the maytansinoid to a linker of a conjugate, such as e.g. an antibody drug conjugate. R at each occurrence is independently H or a C₁-C₆ alkyl. The alkylene chain attaching the amide group to the sulfur atom may be methanyl, ethanyl, or propanyl, i.e. m is 1, 2, or 3. (U.S. Pat. No. 633,410, U.S. Pat. No. 5,208,020, Chari et al. (1992) Cancer Res. 52; 127-131, Lui et al. (1996) Proc. Natl. Acad. Sci. 93:8618-8623). Accordingly, in these embodiments the sulphur atom can represent the group X.

All stereoisomers of the maytansinoid drug moiety are contemplated for the conjugates, disclosed herein, i.e. any combination of R and S configurations at the chiral carbons of the maytansinoid. In some embodiments the maytansinoid drug moiety has the following stereochemistry:

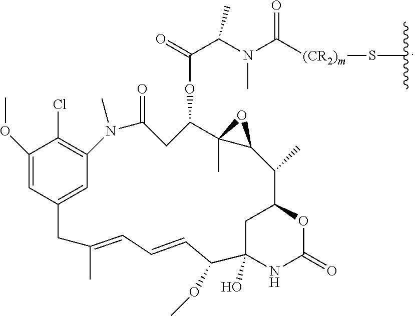

Accordingly, in these embodiments the sulphur atom can represent the group X.

In some embodiments the maytansinoid drug moiety is N^{2′}-deacetyl-N^{2′}-(3-mercapto-1-oxopropyl)-maytansine (also known as DM1). DM1 is represented by the following structural structure:

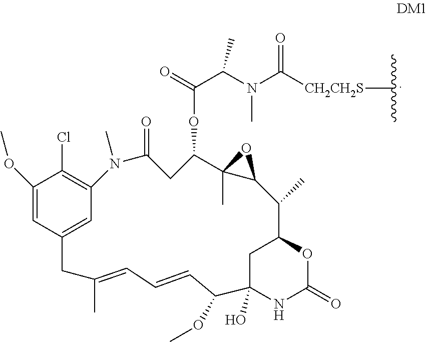

Accordingly, in these embodiments the sulphur atom can represent the group X.

In some embodiments the maytansinoid drug moiety is N^{2′}-deacetyl-N^{2′}-(4-mercapto-1-oxopentyl)-maytansine (also known as DM3). DM3 is represented by the following structural structure:

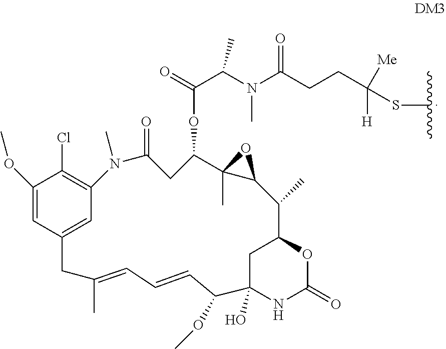 [SMILES: *SC(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

Accordingly, in these embodiments the sulphur atom can represent the group X.

In some embodiments the maytansinoid drug moiety is N^{2′}-deacetyl-N^{2′}-(4-methyl-4-mercapto-1-oxopentyl)-maytansine (also known as DM4). DM4 is represented by the following structure:

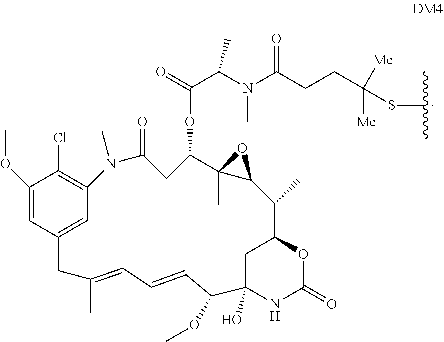 [SMILES: *SC(C)(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

Accordingly, in these embodiments the sulphur atom can represent the group X.

Preferably, in conjugates, disclosed herein comprising a maytansinoid drug moiety the maytansinoid is N^{2′}-deacetyl-N^{2′}-(3-mercapto-1-oxopropyl)-maytansine (DM1) or N^{2′}-deacetyl-N^{2′}-(4-mercapto-4-methyl-1-oxopentyl)-maytansine (DM4).

The drug moiety may be a calicheamicin. “Calicheamicins” as used herein relate to a class of enediyne antitumor antibiotics derived from the bacterium Micromonospora echinospora, with calicheamicin γ1 being the most notable. It was isolated originally in the mid-1980s from the chalky soil, or “caliche pits”, located in Kerrville, Texas. It is extremely toxic to all cells. Accordingly, the drug moiety may be Calicheamicin γ1 exemplified by the following structure, which may be optionally substituted or derivatized for coupling to a linker and/or a receptor binding molecule:

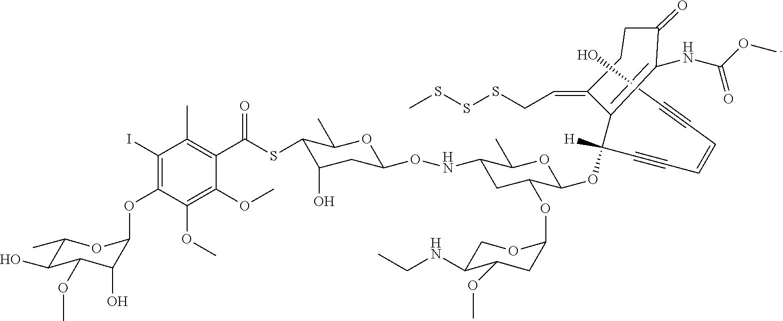

The drug moiety may be a tubulysin. Tubulysins have functions as being anti-microtubule, anti-mitotic, apoptosis inducer, anticancer, anti-angiogenic, and antiproliferative. Tubulysins are cytotoxic peptides, which include 9 members (A-I). Preferably, the tubulysin is Tubulysin A. Tubulysin A has potential application as an anticancer agent. It arrests cells in the G2/M phase. Tubulysin A has the following structure:

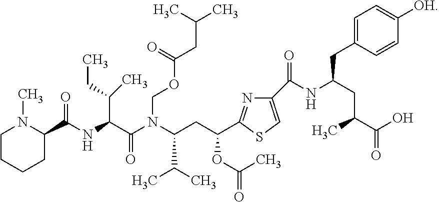 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)O)cs1)C(C)C]

The drug moiety may be an amatoxin. Amatoxin is the collective name of a subgroup of at least eight related toxic compounds found in several genera of poisonous mushrooms, most notably the death cap (Amanita phalloides) and several other members of the genus Amanita, as well as some Conocybe, Galerina and Lepiota mushroom species. Amatoxins are lethal in even small doses. The compounds have a similar structure, that of eight amino-acid residues arranged in a conserved macrobicyclic motif (an overall pentacyclic structure when counting the rings inherent in the proline and tryptophan-derived residues). All amatoxins are oligopeptides that are synthesized as 35-amino-acid proproteins, from which the final eight amino acids are cleaved by a prolyl oligopeptidase. The schematic amino acid sequence of amatoxins is IIe-Trp-Gly-Ile-Gly-Cys-Asn-Pro (SEQ ID NO: 1) with cross-linking between Trp and Cys via the sulfoxide (S═O) moiety and hydroxylation in variants of the molecule. There are currently ten known amatoxins, which might be the drug moiety:

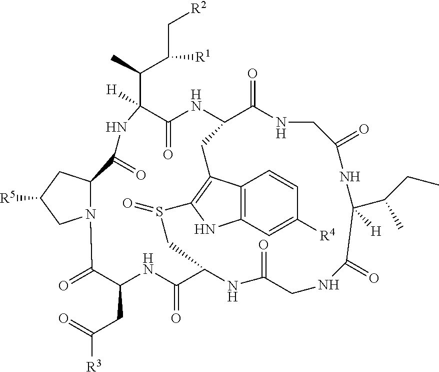

| Name | R¹ | R² | R³ | R⁴ | R⁵ |
|---|---|---|---|---|---|
| α-Amanitin | OH | OH | NH₂ | OH | OH |
| β-Amanitin | OH | OH | OH | OH | OH |
| γ-Amanitin | OH | H | NH₂ | OH | OH |
| ε-Amanitin | OH | H | OH | OH | OH |
| Amanullin | H | H | NH₂ | OH | OH |
| Amanullinic acid | H | H | OH | OH | OH |
| Amaninamide | OH | OH | NH₂ | H | OH |
| Amanin | OH | OH | OH | H | OH |
| Proamanullin | H | H | NH₂ | OH | H |

The drug moiety may be a dolastatin such as Dolastatin 10 or dolastatin 15. Both are marine natural products isolated from the Indian Ocean sea hare Dollabella auricularia. This potent antitumor agent is also isolated from the marine cyanobacterium Symploca sp. VP642 from Palau. Being a small linear peptide molecules, dolastatin 10 and 15 are considered anti-cancer drugs showing potency against breast and liver cancers, solid tumors and some leukemias. Preclinical research indicated potency in experimental antineoplastic and tubulin assembly systems. The dolastatins are mitotic inhibitors. They inhibit microtubule assembly by interfering with tubulin formation and thereby disrupt cell division by mitosis and induces apoptosis and Bcl-2 phosphorylation in several malignant cell types. Dolostatin 10 (N,N-Dimethyl-L-valyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]-1-pyrrolidinyl}-5-methyl-1-oxo-4-heptanyl]-N-methyl-L-valinamide) has the following structure:

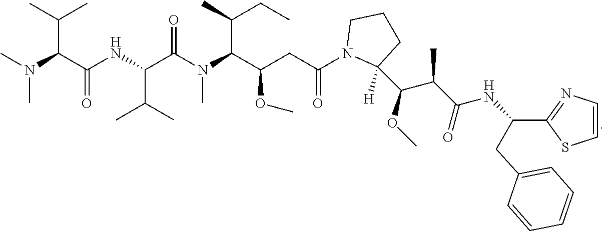 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C)C(C)C]

### Dolastatin 15 ((2S)-1-[(2S)-2-Benzyl-3-methoxy-5-oxo-2,5-dihydro-1 H-pyrrol-1-yl]-3-methyl-1-oxo-2-butanyl N,N-dimethyl-L-valyl-L-valyl-N-methyl-L-valyl-L-prolyl-L-prol) has the following structure

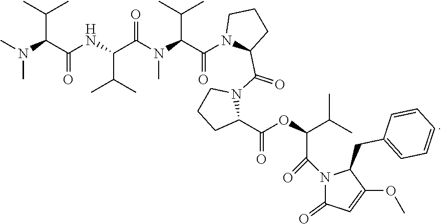 [SMILES: COC1=CC(=O)N(C(=O)[C@@H](OC(=O)[C@@H]2CCCN2C(=O)[C@@H]2CCCN2C(=O)[C@H](C(C)C)N(C)C(=O)[C@@H](NC(=O)[C@H](C(C)C)N(C)C)C(C)C)C(C)C)[C@H]1Cc1ccccc1]

The drug moiety may be a Pyrrolobenzodiazepine Dimer such as a compound having the following structure, which may be optionally substituted or derivatized for coupling to a linker and/or a receptor binding molecule:

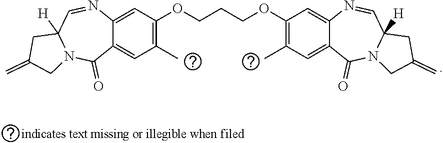

The drug moiety may be a Indolinobenzodiazepin Dimer such as a compound having the following structure:

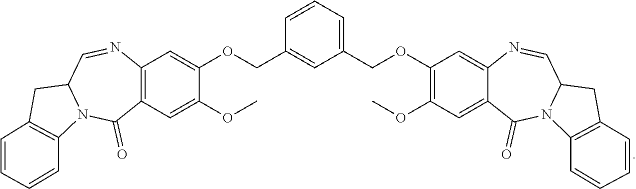 [SMILES: COc1cc2c(cc1OCc1cccc(COc3cc4c(cc3OC)C(=O)N3c5ccccc5CC3C=N4)c1)N=CC1Cc3ccccc3N1C2=O]

The drug moiety may be a nicotinamide phosphoribosyltransferase (Nampt) inhibitor The Nampt inhibitor may be Nampt-IN-1. In some preferred embodiments, the drug moiety is Nampt-IN-1. Nampt-IN-1 has the following structure:

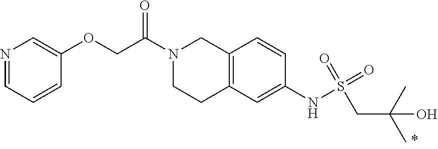 [SMILES: CC(C)(O)CS(=O)(=O)Nc1ccc2c(c1)CCN(C(=O)COc1cccnc1)C2]

Preferably, in any one of these embodiments the Nampt-IN-1 may be bound to the phosphorus atom the hydroxy group marked with an asterisk (*). Accordingly, in these embodiments the oxygen atom of the hydroxy group marked with the asterisk represents the group X.

The drug moiety may be a radioisotope. Typical radioisotopes as described herein may relate to a small radiation source, usually a gamma or beta emitter such as iodine-125, iodine-131, iridium-192 or palladium-103.

The drug moiety may be a therapeutic protein or peptide or a fragment thereof. Typical examples are cytokines such as interleukines, ricin, diphtheria toxin, Pseudomonas exotoxin PE38.

The drug moiety may be a KSP (kinesin spindle protein) inhibitor. Examples of KSP inhibitors include Ispinesib (SB-715992), SB743921, AZ 3146, GSK923295, BAY 1217389, MPI-0479605 and ARQ 621.

The drug moiety may be an inhibitor of eukaryotic Translation Initiation Factor 4E (eIF4E). Examples of eIF4E inhibitors include ON-013100. The structure of ON-013100 is as depicted below:

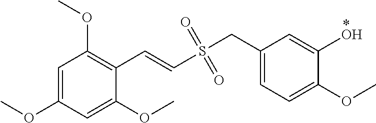

ON-013100 can be bound to the phosphorus, e.g., via the terminal OH group (marked with an asterisk *); accordingly, in these embodiments the O of the OH group represents the group X.

The drug moiety may be an inhibitor of dihydroorotate dehydrogenase (DHODH). Examples of DHODH inhibitors include Bay-2402234 and DHODH-IN-16. Bay-2402234 has the following structure:

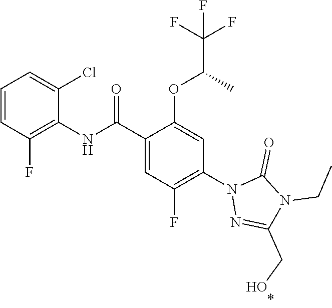 [SMILES: CCn1c(CO)nn(-c2cc(O[C@@H](C)C(F)(F)F)c(C(=O)Nc3c(F)cccc3Cl)cc2F)c1=O]

Bay-2402234 may be bound to the phosphorus, e.g., via the terminal OH group (marked with an asterisk *); accordingly, in these embodiments the O of the OH group represents the group X.

DHODH-IN-16 has the following structure:

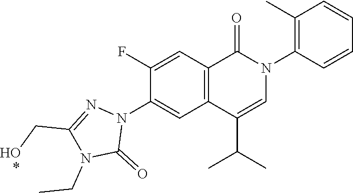 [SMILES: CCn1c(CO)nn(-c2cc3c(C(C)C)cn(-c4ccccc4C)c(=O)c3cc2F)c1=O]

DHODH-IN-16 can be bound to the phosphorus, e.g., via the terminal OH group (marked with an asterisk *); accordingly, in these embodiments the O of the OH group represents the group X.

The drug moiety may be a taxane. Taxanes are a class of chemotherapeutic agents that act by binding to tubulins/microtubules thereby causing cell cycle inhibition during the G2/M phase, which plays a key role in cell division. Examples of taxanes include Paclitaxel, albumin-bound Paclitaxel (nab-Paclitaxel), Docetaxel, Cabazitaxel and Abraxan. In a preferred embodiment, the taxane is Paclitaxel. The structure of Paclitaxel is depicted below:

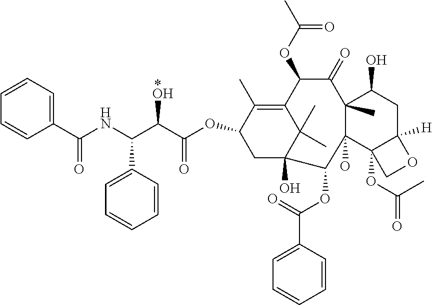

Paclitaxel may be bound to the phosphorus, e.g., via one of the OH groups; accordingly, in these embodiments the O of the OH group represents the group X. In generally preferred embodiments, the Pacliataxel can be bound via the OH group marked with an asterisk *.

### Group M and Linker L

The present disclosure provides conjugates, where a receptor binding molecule, as described herein, is linked to a drug moiety. In accordance with the present disclosure, the receptor binding molecule may be linked, inter alia, via the group M and covalent attachment by a linker L, to the drug moiety. As used herein, a “linker” L is any chemical moiety that is capable of linking a group M, such as e.g. oxygen (O), to the receptor binding molecule. In this regard, it is again referred to the formula (I) described herein:

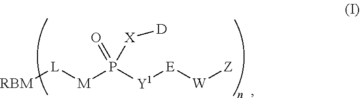

Accordingly, the receptor binding molecule can be linked to M through a linker L. In formula (I), RBM, L, M, X, D, Y¹, E, W, Z and n are as defined herein.

The group M may be O, NR^{M60}, S or CR^{M61}R^{M6}2. R^{M60} may be selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{M60} is hydrogen or (C₁-C₈)alkyl. More preferably, R^{M60} is hydrogen or (C₁-C₆)alkyl, still more preferably hydrogen or (C₁-C₄)alkyl, even more preferably hydrogen or (C₁-C₂)alkyl. In preferred embodiments, R^{M60} is hydrogen. R^{M61} and R^{M62} may be each independently selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{M61} and R^{M62} are each independently selected from the group consisting of hydrogen or (C₁-C₈)alkyl. More preferably, R^{M61} and R^{M62} are each independently hydrogen or (C₁-C₆)alkyl, still more preferably hydrogen or (C₁-C₄)alkyl, even more preferably hydrogen or (C₁-C₂)alkyl. In preferred embodiments, R^{M61} and R^{M62} are hydrogen. In some embodiments, M is O or NR^{M60} wherein R^{M60} is as defined herein. In some embodiments, M is NR^{M60}, wherein R^{M60} is as defined herein. In some embodiments, M is S (sulfur). In some embodiments, M is CR^{M61}R^{M6}2, wherein R^{M61} and R^{M6}2 are as defined herein.

In some preferred embodiments, M is O.

In some preferred embodiments, M is NH.

In some preferred embodiments, M is O, X is O and Y¹ is NR^{A20}, wherein R^{A20} is as defined herein. In more preferred embodiments, M is O, X is O and Y¹ is NH. In any one of these embodiments, the integer m may be 0. Preferably, in any one of these embodiments the drug moiety may be a camptothecin compound. More preferably, the camptothecin compound may be SN38 or DXD. Still more preferably, the camptothecin compound may be SN38.

The linker L serves to connect the moiety M with the receptor binding molecule (RBM). The linker L is any chemical moiety that is capable of linking M to the receptor binding molecule (RBM). In particular, the linker L attaches M to the receptor binding molecule (RBM) through covalent bond(s). The linker reagent is a bifunctional or multifunctional moiety which can be used to link a receptor binding molecule (RBM) and M to form conjugates of formula (I). The terms “linker reagent”, “cross-linking reagent”, “linker derived from a cross-linking reagent” and “linker” may be used interchangeably throughout the present disclosure. Preferably, the linker is substantially resistant to cleavage, e.g., the linker is a stable linker or non-cleavable linker. A non-cleavable linker is any chemical moiety capable of linking a receptor binding molecule (RBM) to M in a stable, covalent manner. In particular, non-cleavable linkers are substantially resistant to acid-induced cleavage, photo-induced cleavage, peptidase-induced cleavage, protease-induced cleavage, glycosidase-induced cleavage, phosphatase-induced cleavage, esterase-induced cleavage and disulfide bond cleavage. Furthermore, “non-cleavable” in particular refers to the ability of the chemical bond in the linker or adjoining to the linker to withstand cleavage induced by an acid, photo labile-cleaving agent, a peptidase, a protease, a glycosidase, a phosphatase, an esterase, or a chemical or physiological compound that cleaves a disulfide bond, at conditions under which the drug moiety or the receptor binding molecule does not lose its activity.

Virtually any linker can be used. The linker may, for example, be a straight or branched hydrocarbon based moiety. The linker can also comprise cyclic moieties. If the linking moiety is a hydrocarbon-based moiety the main chain of the linker may comprise only carbon atoms but can also contain heteroatoms such as oxygen (O), nitrogen (N) or sulfur (S) atoms. The linker may for example include a (C₁-C₂₀) carbon atom chain or a polyether based chain such as a polyethylene glycol based chain with —(O—CH₂—CH₂)— repeating units. In typical embodiments of hydrocarbon based linkers, the linking moiety may comprise between 1 to about 150, 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18, and 19 main chain atoms.

A receptor binding molecule (RBM) has a functional group that can form a bond with a functional group of the linker (L). Useful functional groups that can be present on a receptor binding molecule, either naturally or via chemical manipulation include, but are not limited to, sulfhydryl (—SH), amino, hydroxyl, carboxy, the anomeric hydroxyl group of a carbohydrate, and carboxyl. Preferred functional groups of the receptor binding molecule are sulhydryl and amino. Sulfhydryl groups can be generated, e.g., by reduction of an intramolecular disulfide bond of a receptor binding molecule. Suitable reducing agents for reducing disulfide bonds to sulfhydryl groups are known to a person skilled in the art and include, as non-limiting examples, tris(2-carboxyethyl)phosphine (TCEP), dithiothreitol (DTT), sodium dithionite, sodium thiosulfate, and sodium sulfite. As illustrative example, the receptor binding molecule may be an antibody, which comprises one or more disulfide bonds that can be reduced to give sulfhydryl groups. Alternatively, sulfhydryl groups can be generated, e.g., by reaction of an amino group of a lysine moiety of a receptor binding molecule using 2-iminothiolane (Traut's reagent) or another sulfhydryl generating reagent. It is also possible to generate sulhydryl groups by the (genetic) incorporation of extra cysteine residues into the structure of the receptor binding molecule.

In some embodiments, the linker forms a bond with a sulfur atom of the receptor binding molecule. The sulfur atom can be derived from a sulfhydryl group of a receptor binding molecule. Preferably, the receptor binding molecule is an antibody. Representative linkers bound to a receptor binding molecule (RBM) are depicted in formulas (IIIa) and (IIIb), wherein Q is a connector unit and # indicates the attachment to the group M. Such linkers are described, e.g., in WO 2004/010957.

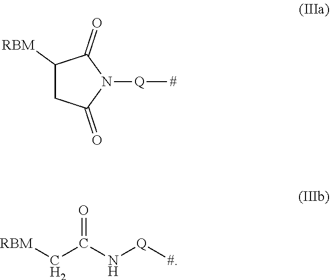

In some embodiments, the linker contains a functional group that can form a bond with a primary or secondary amino group of a receptor binding molecule. Examples of such functional groups include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative linkers bound to a receptor binding molecule (RBM) are depicted in formulas (Va), (Vb) and (Vc), wherein Q is a connector unit and # indicates the attachment to the group M. Such linkers are described, e.g., in WO 2004/010957.

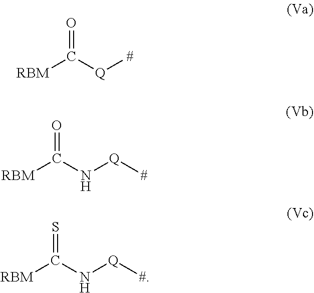

In some embodiments, the linker contains a functional group that can form a bond with an aldehyde group (denoted herein as —CHO or

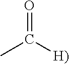

of a carbohydrate that can be present on the receptor binding molecule. For example, a carbohydrate can be mildly oxidized using a reagent such as sodium periodate, and the resulting —CHO group of the oxidized carbohydrate can be condensed with a linker that contains a functional group such as e.g. a hydrazide, an oxime, a primary or secondary amine, a hydrazine, a thiosemicarbazone, a hydrazine carboxylate, and an arylhydrazide such as those described by Kaneko, T. et al. Bioconjugate Chem. 1991, 2, 133-41. Representative linkers bound to a receptor binding molecule (RBM) are depicted in formulas (VIa), (VIb) and (VIc), wherein Q is a connector unit and # indicates the attachment to the group M. Such linkers are described, e.g., in WO 2004/010957.

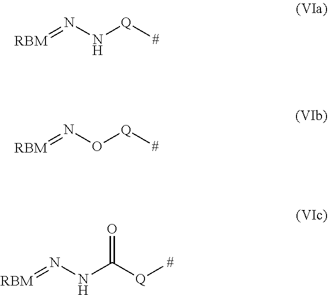

The term “connector unit Q”, whenever used herein, refers to a chemical moiety which forms part of the linker L and serves to connect the linker to the group M. Any chemical moiety, which is capable to form a bond with M can be used. In some embodiments, whenever referred to a connector unit Q herein, the connector unit Q may be selected from the group consisting of —(C₁-C₁₀)alkylene-, —(C₃-C₈)carbocyclo-, -arylene-, —(C₁-C₁₀)alkylene-arylene-^{#}, -arylene-(C₁-C₁₀)alkylene-^{#}, —(C₁-C₁₀)alkylene-(C₃-C₈)carbocyclo-^{#}, —(C₃-C₈)carbocyclo-(C₁-C₁₀)alkylene-^{#}, —(C₃-C₈)heterocyclo-, —(C₁-C₁₀)alkylene-(C₃-C₈)heterocyclo-^{#}, —(C₃-C₈)heterocyclo-(C₁-C₁₀)alkylene-^{#} and —(CH₂CH₂O)ᵣCH₂—CH₂-^{#}, wherein r is an integer ranging from 1 to 9; #, when present, denotes the attachment to M. Each connector unit may be optionally substituted.

In preferred embodiments, Q is (C₂-C₁₀)alkylene; more preferably (C₂-C₈)alkylene; still more preferably (C₂-C₆)alkylene. Even more preferably, Q may be —(CH₂)_{q}—, wherein q is an integer ranging from 2 to 10, preferably from 2 to 8, more preferably from 3 to 6, still more preferably from 4 to 5; even more preferably q is 5.

In preferred embodiments, Q is (C₃-C₈)carbocycle, C₆-C₁₀)aryl (phenyl), a five-or six-membered heterocyclic ring comprising 1, 2 or 3 heteroatoms independently selected from the group consisting of N, O and S, more preferably (C₃-C₈)cycloalkyl or (C₅-C₈)cycloalkenyl, still more preferably 5-, 6-, or 7-membered cycloalkyl, even more preferably cyclohexyl. In some embodiments, Q is cyclohexyl.

In preferred embodiments, Q is:

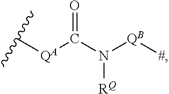

wherein:
Q^{A} is, independently, (C₁-C₁₀)alkylene; preferably (C₁-C₈)alkylene; more preferably (C₂-C₅)alkylene
Q^{B} is, independently, (C₂-C₁₀)alkylene; preferably (C₂-C₈) alkylene; more preferably (C₂-C₅)alkylene;
R^{Q} is selected from the group consisting of hydrogen, (C₁-C₈)alkyl (e.g. methyl, ethyl or propyl), (C₆-C₁₀)aryl (phenyl), and (C₁-C₈)alkylene(C₆-C₁₀)aryl (e.g. benzyl); preferably, R^{Q} is hydrogen or (C₁-C₈)alkyl; more preferably, R^{Q} is hydrogen or (C₁-C₆)alkyl; still more preferably, R^{Q} is hydrogen or (C₁-C₄)alkyl; still more preferably, R^{Q} is hydrogen or (C₁-C₂)alkyl; even more preferably, R^{Q} is hydrogen;
# indicates the attachment to M; and
[symbol] indicates the attachment to the remainder of L.

More preferably, Q is:

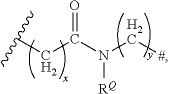

wherein:
R^{Q} is selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl); preferably, R^{Q} is hydrogen or (C₁-C₈)alkyl; more preferably, R^{Q} is hydrogen or (C₁-C₆)alkyl; still more preferably, R^{Q} is hydrogen or (C₁-C₄)alkyl; still more preferably, R^{Q} is hydrogen or (C₁-C₂)alkyl; even more preferably, R^{Q} is hydrogen;
x is an integer independently ranging from 1 to 10; preferably from 2 to 8; more preferably from 3 to 6; still more preferably from 4 to 6; in some preferred embodiments x is 5;
y is an integer independently ranging from 2 to 10; preferably from 2 to 8; more preferably from 3 to 6; still more preferably from 4 to 6; in some preferred embodiments y is 5;
# indicates the attachment to M; and
[symbol] indicates the attachment to the remainder of L.

In preferred embodiments, Q is:

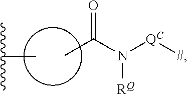

[symbol] is a five- or six-membered carbocyclic ring; or
[symbol] is a five- or six-membered heterocyclic ring comprising 1, 2 or 3 heteroatoms independently selected from the group consisting of N, O and S;
R^{Q} is selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl); preferably, R^{Q} is hydrogen or (C₁-C₈)alkyl; more preferably, R^{Q} is hydrogen or (C₁-C₆)alkyl; still more preferably, R^{Q} is hydrogen or (C₁-C₄)alkyl; still more preferably, R^{Q} is hydrogen or (C₁-C₂)alkyl; even more preferably, R^{Q} is hydrogen;

Q^{C} is (C₂-C₂o)alkylene; preferably (C₂-C₁₀)alkylene; more preferably (C₂-C₈)alkylene; even more preferably (C₂-C₆)alkylene; or (C₃-C₈)carbocycle, (C₆-C₁₀)aryl (phenyl), a five- or six-membered heterocyclic ring comprising 1, 2 or 3 heteroatoms independently selected from the group consisting of N, O and S, (C₃-C₈)cycloalkyl; preferably 5-, 6-, or 7-membered cycloalkyl, even more preferably cyclohexyl;
# indicates the attachment to M; and
[symbol] indicates the attachment to the remainder of L.

In preferred embodiments, whenever mentioned herein, Q^{C} is (CH₂)ₚ, wherein p is an integer ranging from 2 to 20, preferably from 2 to 10, more preferably from 3 to 8, still more preferably from 4 to 6; in some preferred embodiments p is 5.

The carbocyclic ring may be aromatic or non-aromatic. The heterocyclic ring may be aromatic or non-aromatic.

More preferably, Q is selected from the group consisting of

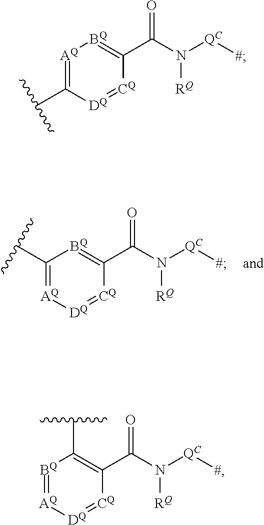

wherein each of A^{Q}, B^{Q}, C^{Q} and D^{Q} is independently selected from N (nitrogen) and C—H; preferably, at least one of A^{Q}, B^{Q}, C^{Q} and D^{Q} is C—H; more preferably, at least two of A^{Q}, B^{Q}, C^{Q} and D^{Q} are C—H; still more preferably, at least three of A^{Q}, B^{Q}, C^{Q} and D^{Q} are C—H, even more preferably, each of A^{Q}, B^{Q}, C^{Q} and D^{Q} are C—H; R^{Q} is as defined herein; preferably R^{Q} is hydrogen; Q^{C} is as defined herein; #denotes the attachment to M; and [symbol] indicates the attachment to the remainder of L.

Still more preferably, Q is selected from the group consisting of

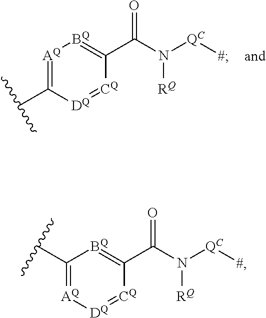

wherein each of A^{Q}, B^{Q}, C^{Q} and D^{Q} is independently selected from N (nitrogen) and C—H; preferably, at least one of A^{Q}, B^{Q}, C^{Q} and D^{Q} is C—H; more preferably, at least two of A^{Q}, B^{Q}, C^{Q} and D^{Q} are C—H; still more preferably, at least three of A^{Q}, B^{Q}, C^{Q} and D^{Q} are C—H, even more preferably, each of A^{Q}, B^{Q}, C^{Q} and D^{Q} are C—H; R^{Q} is as defined herein; preferably R^{Q} is hydrogen; Q^{C} is as defined herein; #denotes the attachment to M; and [symbol] indicates the attachment to the remainder of L.

Even more preferably, Q is:

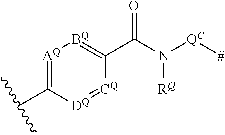

wherein each of A^{Q}, B^{Q}, C^{Q} and D^{Q} is independently selected from N (nitrogen) and C—H; preferably, at least one of A^{Q}, B^{Q}, C^{Q} and D^{Q} is C—H; more preferably, at least two of A^{Q}, B^{Q}, C^{Q} and D^{Q} are C—H; still more preferably, at least three of A^{Q}, B^{Q}, C^{Q} and D^{Q} are C—H, even more preferably, each of A^{Q}, B^{Q}, C^{Q} and D^{Q} are C—H; R^{Q} is as defined herein; preferably R^{Q} is hydrogen; Q^{C} is as defined herein; #denotes the attachment to M; and [symbol] indicates the attachment to the remainder of L.

In some preferred embodiments, Q is:

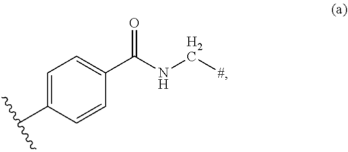

wherein p is an integer ranging from 2 to 20, preferably from 2 to 10, more preferably from 3 to 8, still more preferably from 4 to 6; in some preferred embodiments p is 5; # indicates the attachment to M; and [symbol] indicates the attachment to the remainder of L; or

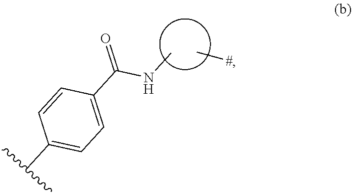

wherein [symbol] is a (C₃-C₈)carbocycle, (C₆-C₁₀)aryl (phenyl), a five- or six-membered heterocyclic ring comprising 1, 2 or 3 heteroatoms independently selected from the group consisting of N, O and S; preferably (C₃-C₈)cycloalkyl; more preferably 5-, 6-, or 7-membered cycloalkyl, even more preferably cyclohexyl; # indicates the attachment to M; and [symbol] indicates the attachment to the remainder of L.

In preferred embodiments, the linker L is:

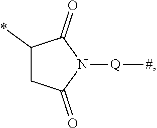

wherein Q is as defined herein, the asterisk (*) indicates the attachment to the receptor binding molecule (RBM) and # indicates the attachment to the group M.

Preferably, the linker is attached to the receptor binding molecule (RBM) via a sulfur atom (S). Accordingly, in such embodiments a combination of the linker and the receptor binding molecule can be depicted as follows:

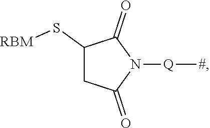

wherein RBM and Q are as defined herein and # indicates the attachment to the group M.

Preferably, in any one of these embodiments the receptor binding molecule is an antibody. The sulfur atom, which provides the bonding to the linker L, may result from a sulfhydryl group, which may be obtained by reduction of a disulfide bond of the antibody.

In any one of these embodiments, the connector unit Q may be any connector unit Q as defined herein. Accordingly, in any one of these embodiments Q may be (C₂-C₁₀)alkylene; more preferably (C₂-C₈)alkylene; still more preferably (C₂-C₆)alkylene; still more preferably, Q may be —(CH₂)_{q}—, wherein q is an integer ranging from 2 to 10, preferably from 2 to 8, more preferably from 3 to 6, still more preferably from 4 to 5; even more preferably q is 5. It is also possible that in any one of these embodiments Q is (C₆-C₁₀)arylene, such as, for example, phenylene (e.g., 1,4-phenylene).

Preferably, in any one of these embodiments Q is:

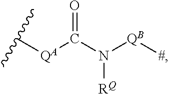

wherein Q^{A}, R^{Q} and Q^{B} are as defined herein; # indicates the attachment to M; and [symbol] indicates the attachment to the remainder of L. Accordingly, in these embodiments the linker L has the following structure:

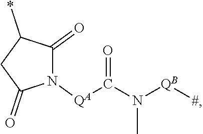

wherein Q^{A}, R^{Q} and Q^{B} are as defined herein; the asterisk (*) indicates the attachment to the receptor binding molecule (RBM) and # indicates the attachment to M.

More preferably, in any one of these embodiments Q is:

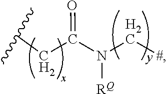

wherein x, R^{Q} and y are as defined herein; preferably R^{Q} is hydrogen; # indicates the attachment to M; and [symbol] indicates the attachment to the remainder of L. Accordingly, in these embodiments the linker L has the following structure:

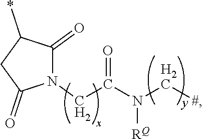

wherein x, R^{Q} and y are as defined herein; preferably R^{Q} is hydrogen; the asterisk (*) indicates the attachment to the receptor binding molecule (RBM) and # indicates the attachment to M.

In some embodiments, the linker L is:

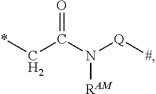

wherein:
R^{AM} is selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl); preferably, R^{AM} is hydrogen or (C₁-C₈)alkyl; more preferably R^{AM} is hydrogen or (C₁-C₆)alkyl; still more preferably R^{AM} is hydrogen or (C₁-C₄)alkyl; still more preferably R^{AM} is hydrogen or (C₁-C₂)alkyl; even more preferably R^{AM} is hydrogen;
Q is as defined herein;
the asterisk (*) indicates the attachment to the receptor binding molecule (RBM); and
# indicates the attachment to the group M.

Preferably, the linker is attached to the receptor binding molecule (RBM) via a sulfur atom (S). Accordingly, in such embodiments a combination of the linker and the receptor binding molecule can be depicted as follows:

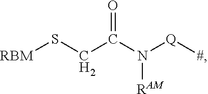

wherein RBM, R^{AM} and Q are as defined herein and # indicates the attachment to the group M.

Preferably, in any one of these embodiments the receptor binding molecule is an antibody. The sulfur atom, which provides the bonding to the linker L, may result from a sulfhydryl group, which may be obtained by reduction of a disulfide bond of the antibody.

In any one of these embodiments, the connector unit Q may be any connector unit Q as defined herein. In particular, in any one of these embodiments Q may be (C₂-C₁₀)alkylene; more preferably (C₂-C₈)alkylene; still more preferably (C₂-C₆)alkylene; still more preferably, Q may be —(CH₂)_{q}—, wherein q is an integer ranging from 2 to 10, preferably from 2 to 8, more preferably from 3 to 6, still more preferably from 4 to 5; even more preferably q is 5. It is also possible that in any one of these embodiments Q is (C₆-C₁₀)arylene, such as, for example, phenylene (e.g., 1,4-phenylene).

Preferably, in any one of these embodiments the linker L has the following structure:

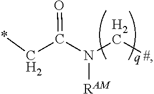

wherein:
R^{AM} is as defined herein; accordingly, R^{AM} is selected from the group consisting of hydrogen, (C₁-C₈)alkyl (e.g. methyl, ethyl or propyl), (C₆-C₁₀)aryl (e.g. phenyl), and (C₁-C₈)alkylene (C₆-C₁₀)aryl (e.g. benzyl); preferably, R^{AM} is hydrogen or (C₁-C₈)alkyl; more preferably R^{AM} is hydrogen or (C₁-C₆)alkyl; still more preferably R^{AM} is hydrogen or (C₁-C₄)alkyl; still more preferably R^{AM} is hydrogen or (C₁-C₂)alkyl; even more preferably R^{AM} is hydrogen;
q is as defined herein; accordingly, q is an integer ranging from 2 to 10, preferably from 2 to 8, more preferably from 3 to 6, still more preferably from 4 to 5; even more preferably q is 5; the asterisk (*) indicates the attachment to the receptor binding molecule (RBM); and
# indicates the attachment to M.

In preferred embodiments, the linker L has the formula (L-I):

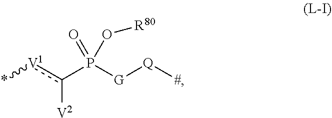

wherein:
[symbol] is a double bond; or
[symbol] is a bond;
V² is absent when [symbol] is a double bond; or
V² is selected from the group consisting of hydrogen, (C₁-C₈)alkyl, (C₆-C₁₀)aryl, and (C₁-C₈)alkylene(C₆-C₁₀)aryl when [symbol] is a bond;
V¹ is R^{V11—C} when [symbol] is a double bond; or
V¹ is

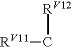

when [symbol] is a bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl;
R^{C50} is an optionally substituted aliphatic residue or an optionally substituted aromatic residue; the asterisk (*) indicates the attachment to the receptor binding molecule (RBM); and
# indicates the attachment to the group M.
Phosphonamidate, phosphonothiolate and phosphonate moieties, which are comprised in the linker (L-I), and their preparations are generally known, e.g., from WO 2018/041985 and WO 2019/170710, which are hereby incorporated by reference in its entirety.

Preferably, the linker L-I is attached to the receptor binding molecule (RBM) via a sulfur atom (S). In such embodiments a combination of the linker ((L-I) and the receptor binding molecule can be depicted as follows:

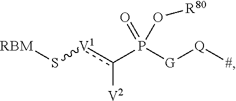

wherein RBM, V¹, V², R⁸⁰, G and Q are as defined herein; and # indicates the attachment to the group M. Accordingly, in some embodiments the conjugate has formula (Ia2):

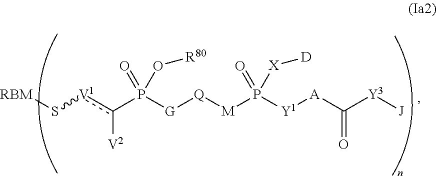

or a pharmaceutically acceptable salt or solvate thereof;
wherein RBM, V¹, V², [symbol] , R⁸⁰, G, Q, M, X, D, Y¹, A, Y³, J and n are as defined herein. Preferably, in any one of these embodiments the receptor binding molecule is an antibody. The sulfur atom, which provides the bonding to the linker L, may result from a sulfhydryl group, which may be obtained by reduction of a disulfide bond of the receptor binding molecule (RBM), e.g. an antibody.

Preferably R^{V11} is H or (C₁-C₈)alkyl; more preferably R^{V11} is H. Preferably R^{V12}, when present is H or (C₁-C₈)alkyl; more preferably R^{V12}, when present, is H. Preferably R^{G70}, when present is H or (C₁-C₈)alkyl; more preferably R^{G70}, when present, is H. Preferably R^{G71}, when present is H or (C₁-C₈)alkyl; more preferably R^{G71}, when present, is H. Preferably R^{G72} when present is H or (C₁-C₈)alkyl; more preferably R^{G72}, when present, is H.

Preferably, [symbol] is a double bond; V² is absent; V¹ is R^{V11—C} and R^{V11} is selected from the group consisting of hydrogen, (C₁-C₈)alkyl, (C₆-C₁₀)aryl, and (C₁-C₈)alkylene(C₆-C₁₀)aryl; preferably R^{V11} is hydrogen or (C₁-C₈)alkyl; more preferably R^{V11} is hydrogen.

More preferably, [symbol] is a double bond; V² is absent; V¹ is R^{V11—C} and R^{V11} is H or (C₁-C₈)alkyl. Preferably, R^{V11} is H or (C₁-C₆)alkyl, more preferably H or (C₁-C₄)alkyl, still more preferably H or (C₁-C₂)alkyl. In preferred embodiments, R₃ is H.

In some embodiments, [symbol] is a bond; V² is selected from the group consisting of hydrogen, (C₁-C₈)alkyl, (C₆-C₁₀)aryl, and (C₁-C₈)alkylene(C₆-C₁₀)aryl; preferably, V² is hydrogen or (C₁-C₈)alkyl; more preferably, V² is hydrogen; V¹ is

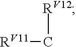

R^{V11} is selected from the group consisting of hydrogen, (C₁-C₈)alkyl, (C₆-C₁₀)aryl, and (C₁-C₈)alkylene(C₆-C₁₀)aryl; preferably R^{V11} is hydrogen or (C₁-C₈)alkyl, more preferably R^{V11} is hydrogen; R^{V12} is selected from the group consisting of hydrogen, (C₁-C₈)alkyl, (C₆-C₁₀)aryl, and (C₁-C₈)alkylene(C₆-C₁₀)aryl; preferably R^{V12} is hydrogen or (C₁-C₈)alkyl, more preferably R^{V12} is hydrogen.

In some embodiments, [symbol] is a bond; V² may be H or (C₁-C₈)alkyl; V¹ is

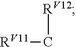

and R^{V11} and R^{V12} may independently be H or (C₁-C₈)alkyl. Preferably, R^{V11} and R^{V12} independently represent H or (C₁-C₆)alkyl, more preferably H or (C₁-C₄)alkyl, still more preferably H or (C₁-C₂)alkyl. Preferably, R^{V11} and R^{V12} are the same; even more preferably, R^{V11}, R^{V12} and V² are the same. More preferably, R^{V11} and R^{V12} are both H. Preferably, V² is H or (C₁-C₆)alkyl, more preferably H or (C₁-C₄)alkyl, still more preferably H or (C₁-C₂)alkyl. Even more preferably, V² is H. In preferred embodiments, R^{V11}, R^{V12} and V² are each H.

The group G is selected from the group consisting of NR^{G70}, S, O, and CR^{G71}R^{G72}. R^{G70} is selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl). Preferably, R^{G70} is hydrogen or (C₁-C₈)alkyl. More preferably, R^{G70} is hydrogen or (C₁-C₆)alkyl, still more preferably hydrogen or (C₁-C₄)alkyl, even more preferably hydrogen or (C₁-C₂)alkyl. In preferred embodiments, R^{G70} is hydrogen. R^{G71} and R^{G72} may be each independently selected from the group consisting of hydrogen, (C₁-C₈)alkyl (e.g. methyl, ethyl or propyl), (C₆-C₁₀)aryl (phenyl), and (C₁-C₈)alkylene(C₆-C₁₀)aryl (e.g. benzyl). Preferably, R^{G71} and R^{G72} are each independently selected from the group consisting of hydrogen or (C₁-C₈)alkyl. More preferably, R^{G71} and R^{G72} are each independently hydrogen or (C₁-C₆)alkyl, still more preferably hydrogen or (C₁-C₄)alkyl, even more preferably hydrogen or (C₁-C₂)alkyl. In preferred embodiments, R^{G71} and R^{G72} are hydrogen.

The group G may be selected from the group consisting of S, O and CR^{G71}R^{G72}, wherein R^{G71} and R^{G72} are as defined herein. G may be S or O. In some embodiments, G is CH₂. In some embodiments, G is O. In some embodiments, G is S.

In preferred embodiments, G is NR^{G70}, wherein R^{G70} is as defined herein. In more preferred embodiments, G is NH.

In a linker having the formula (L-I), the connector unit Q serves to connect the group G to the group M. Any chemical moiety, which is capable to connect G with M, can be used. In particular, in a linker having the formula (L-I), the connector unit Q can be any connector unit Q as described herein.

In preferred embodiments, in a linker having the formula (L-I) the connector unit Q is:

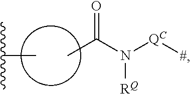

wherein:
[symbol] is a five- or six-membered carbocyclic ring; or
[symbol] is a five- or six-membered heterocyclic ring comprising 1, 2 or 3 heteroatoms independently selected from the group consisting of N, O and S;
R^{Q} is selected from the group consisting of hydrogen, (C₁-C₈)alkyl (e.g. methyl, ethyl or propyl), (C₆-C₁₀)aryl (e.g. phenyl), and (C₁-C₈)alkylene(C₆-C₁₀)aryl (e.g. benzyl); preferably, R^{Q} is hydrogen or (C₁-C₈)alkyl; more preferably, R^{Q} is hydrogen or (C₁-C₆)alkyl; still more preferably, R^{Q} is hydrogen or (C₁-C₄)alkyl; still more preferably, R^{Q} is hydrogen or (C₁-C₂)alkyl; even more preferably, R^{Q} is hydrogen;
Q^{C} is (C₂-C₂₀)alkylene; preferably (C₂-C₁₀)alkylene; more preferably (C₂-C₈)alkylene; even more preferably (C₂-C₆)alkylene; or (C₃-C₈)carbocycle, (C₆-C₁₀)aryl (phenyl) or a five- or six-membered heterocyclic ring comprising 1, 2 or 3 heteroatoms independently selected from the group consisting of N, O and S; preferably (C₃-C₈)cycloalkyl; more preferably 5-, 6-, or 7-membered cycloalkyl, even more preferably cyclohexyl;
# indicates the attachment to M; and
[symbol] indicates the attachment to G.

Accordingly, the linker (L-I) may have the structure:

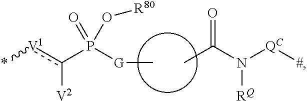

wherein V¹, V², R⁸⁰, G, [symbol] , R^{Q} and Q^{C} are as defined herein; * indicates attachment to the receptor binding molecule (RBM); and # indicates attachment to the group M.

In preferred embodiments, Q^{C} is (CH₂)ₚ, wherein p is an integer ranging from 2 to 20, preferably from 2 to 10, more preferably from 3 to 8, still more preferably from 4 to 6; in some preferred embodiments p is 5.

The carbocyclic ring may be aromatic or non-aromatic. The heterocyclic ring may be aromatic or non-aromatic.

More preferably, with regard to the linker (L-I) the connector unit Q is selected from the group consisting of

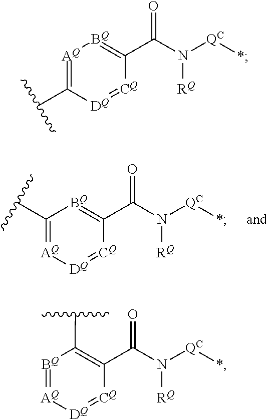

wherein each of A^{Q}, B^{Q}, C^{Q} and D^{Q} is independently selected from N (nitrogen) and C—H; preferably, at least one of A^{Q}, B^{Q}, C^{Q} and D^{Q} is C—H; more preferably, at least two of A^{Q}, B^{Q}, C^{Q} and D^{Q} are C—H; still more preferably, at least three of A^{Q}, B^{Q}, C^{Q} and D^{Q} are C—H, even more preferably, each of A^{Q}, B^{Q}, C^{Q} and D^{Q} are C—H; R^{Q} is as defined herein; preferably R^{Q} is hydrogen; Q_{C} is as defined herein; #denotes the attachment to M; and [symbol] indicates the attachment to G.

Still more preferably, with regard to the linker (L-I), the connector unit Q is selected from the group consisting of

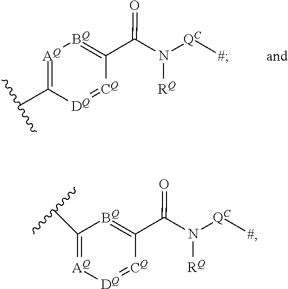

wherein each of A^{Q}, B^{Q}, C^{Q} and D^{Q} is independently selected from N (nitrogen) and C—H; preferably, at least one of A^{Q}, B^{Q}, C^{Q} and D^{Q} is C—H; more preferably, at least two of A^{Q}, B^{Q}, C^{Q} and D^{Q} are C—H; still more preferably, at least three of A^{Q}, B^{Q}, C^{Q} and D^{Q} are C—H, even more preferably, each of A^{Q}, B^{Q}, C^{Q} and D^{Q} are C—H; R^{Q} is as defined herein; preferably R^{Q} is hydrogen; Q^{C} is as defined herein; #denotes the attachment to M; and [symbol] indicates the attachment to G.

Even more preferably, with regard to the linker (L-I), the connector unit Q is:

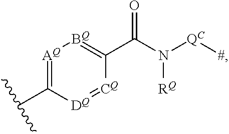

wherein each of A^{Q}, B^{Q}, C^{Q} and D^{Q} is independently selected from N (nitrogen) and C—H; preferably, at least one of A^{Q}, B^{Q}, C^{Q} and D^{Q} is C—H; more preferably, at least two of A^{Q}, B^{Q}, C^{Q} and D^{Q} are C—H; still more preferably, at least three of A^{Q}, B^{Q}, C^{Q} and D^{Q} are C—H, even more preferably, each of A^{Q}, B^{Q}, C^{Q} and D^{Q} are C—H; R^{Q} is as defined herein; preferably R^{Q} is hydrogen; Q^{C} is as defined herein; #denotes the attachment to M; and [symbol] indicates the attachment to G.

In some preferred embodiments of the linker (L-I), the connector unit Q is:

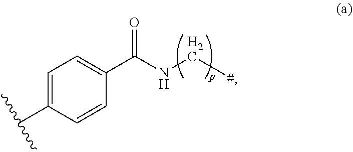

wherein p is an integer ranging from 2 to 20, preferably from 2 to 10, more preferably from 3 to 8, still more preferably from 4 to 6; in some preferred embodiments p is 5; # indicates the attachment to M; and [symbol] indicates the attachment to G; or

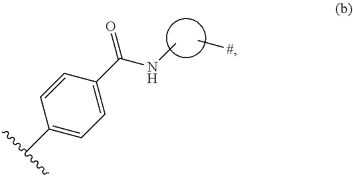

wherein [symbol] is a (C₃-C₈)carbocycle, (C₆-C₁₀)aryl (phenyl) or a five- or six-membered heterocyclic ring comprising 1, 2 or 3 heteroatoms independently selected from the group consisting of N, O and S; preferably (C₃-C₈)cycloalkyl; more preferably 5-, 6-, or 7-membered cycloalkyl, even more preferably cyclohexyl; # indicates the attachment to M; and [symbol] indicates the attachment to G.

The group R^{C50} may be an optionally substituted aliphatic residue or an optionally substituted aromatic residue. Accordingly, R^{C50} covers a broad spectrum of aliphatic or aromatic residues, such as e.g. a group adjusting the water-solubility (e.g. a polyethylene glycol unit). A person skilled in the art knows to select suitable residues R^{C50} which are compatible with the conjugates, compounds, methods and uses described herein.

In some embodiments, R⁸⁰ is optionally substituted (C₁-C₈)alkyl. In particular, R⁸⁰ may be (C₁-C₈)alkyl optionally substituted with at least one of F, Cl, Br, I, —NO₂, —N((C₁-C₈)alkyl)H, —NH₂, —N₃, —N((C₁-C₈)alkyl)₂, ═O, (C₃-C₈)cycloalkyl, (C₂-C₈)alkenyl or (C₂-C₈)alkynyl.

In some embodiments, R⁸⁰ is optionally substituted phenyl. In particular, R⁸⁰ may be phenyl optionally independently substituted with at least one of (C₁-C₈)alkyl, F, Cl, I, Br, —NO₂, —N((C₁-C₈)alkyl)H, —NH₂ or —N((C₁-C₈)alkyl)₂.

In some embodiments, R^{C50} is an optionally substituted 5- or 6-membered heteroaromatic ring such as e.g. pyridyl.

In some embodiments, R⁸⁰ is (C₁-C₈)alkyl, (C₁-C₈)alkyl substituted with optionally substituted phenyl; or phenyl; or phenyl substituted with —NO₂. Preferably, R⁸⁰ is (C₁-C₈)alkyl. More preferably, R⁸⁰ is (C₁-C₆)alkyl. Still more preferably, R^{C50} is (C₁-C₄)alkyl. Even more preferably, R⁸⁰ is (C₁-C₂)alkyl.

In some preferred embodiments, R⁸⁰ is methyl, ethyl, propyl or butyl. More preferably, R⁸⁰ is methyl or ethyl. Still more preferably, R⁸⁰ is ethyl.

In some preferred embodiments, R⁸⁰ is a polyalkylene glycol unit. Polyalkylene glycols, in particular polyethylene glycols, are in principle hydrophilic. Therefore, a polyalkylene glycol unit, in particular a polyethylene glycol unit, can be used, e.g., in order to adjust the hydrophilicity and thus the water-solubility of conjugates described herein.

The term “polyalkylene glycol unit”, as used herein, refers to a polyalkylene glycol unit bound to the O atom, which is attached to the phosphorus (V) moiety of the linker (L-I).

The polyalkylene glycol unit used as R⁸⁰ comprises at least one alkylene glycol subunit. Preferably, the polyalkylene glycol unit used as R⁸⁰ comprises one or more alkylene glycol subunits having the following structure:

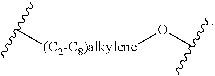

More preferably, the polyalkylene glycol unit used as R⁸⁰ comprises one or more alkylene glycol subunits having the following structure:

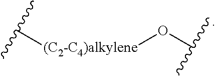

Accordingly, the polyalkylene glycol unit used as R⁸⁰ may be a polytetramethylene glycol unit, a polypropylene glycol unit, or a polyethylene glycol unit. Still more preferably, the polyalkylene glycol unit used as R⁸⁰ comprises one or more alkylene glycol subunits having the following structure:

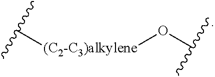

Preferably, the polyalkylene glycol unit used as R⁸⁰ comprises of from 1 to 100 alkylene glycol subunits as described herein. More preferably, the polyalkylene glycol unit used as R⁸⁰ comprises of from 2 to 50 alkylene glycol subunits as described herein. Still more preferably, the polyalkylene glycol unit used as R⁸⁰ comprises of from 3 to 45 alkylene glycol subunits as described herein. Still more preferably, the polyalkylene glycol unit used as R⁸⁰ comprises of from 4 to 40 alkylene glycol subunits as described herein. Still more preferably, the polyalkylene glycol unit used as R⁸⁰ comprises of from 6 to 35 alkylene glycol subunits as described herein. Even more preferably, the polyalkylene glycol unit used as R⁸⁰ comprises of from 8 to 30 alkylene glycol subunits as described herein.

Preferably, the polyalkylene glycol unit used as R⁸⁰ comprises of from 1 to 40 alkylene glycol subunits as described herein. More preferably, the polyalkylene glycol unit used as R⁸⁰ comprises of from 1 to 32 alkylene glycol subunits as described herein. Still more preferably, the polyalkylene glycol unit used as R⁸⁰ comprises of from 2 to 28 alkylene glycol subunits as described herein. In some embodiments, the polyalkylene glycol unit comprises 2, 3, or 4 alkylene glycol subunits as described herein. The polyalkylene glycol unit may comprise 2 or 3, in particular 2, alkylene glycol subunits as described herein. In some embodiments, the polyalkylene glycol unit comprises 10, 11, 12, 13 or 14 alkylene glycol subunits as described herein. The polyalkylene glycol unit may comprise 11, 12 or 13, in particular 12, alkylene glycol subunits as described herein. In some embodiments, the polyalkylene glycol unit comprises 22, 23, 24, 25 or 26 alkylene glycol subunits as described herein. The polyalkylene glycol unit may comprise 23, 24 or 25, in particular 24, alkylene glycol subunits as described herein.

The polyalkylene glycol unit used as R⁸⁰ may be a polyalkylene glycol unit comprising of from 1 to 100, preferably of from 2 to 50, more preferably of from 3 to 45, still more preferably of from 4 to 40, still more preferably of from 6 to 35, even more preferably of from 8 to 30 subunits having the structure:

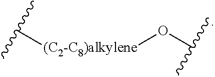

Preferably, the polyalkylene glycol unit used as R⁸⁰ may be a polyalkylene glycol unit comprising of from 1 to 100, preferably of from 2 to 50, more preferably of from 3 to 45, still more preferably of from 4 to 40, still more preferably of from 6 to 35, even more preferably of from 8 to 30 subunits having the structure:

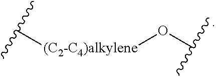

More preferably, the polyalkylene glycol unit used as R⁸⁰ may be a polyalkylene glycol unit comprising of from 1 to 100, preferably of from 2 to 50, more preferably of from 3 to 45, still more preferably of from 4 to 40, still more preferably of from 6 to 35, even more preferably of from 8 to 30 subunits having the structure:

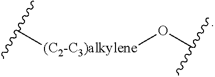

In very preferred embodiments, the polyalkylene glycol unit used as R⁸⁰ may be a polyethylene glycol unit comprising of from 1 to 100, preferably of from 2 to 50, more preferably of from 3 to 45, still more preferably of from 4 to 40, still more preferably of from 6 to 35, even more preferably of from 8 to 30 subunits each having the structure:

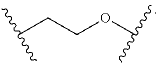 [SMILES: *CCO*]

The polyalkylene glycol unit used as R⁸⁰ may be a polyalkylene glycol unit comprising of from 1 to 40, preferably of from 1 to 32, more preferably of from 2 to 28 subunits having the structure:

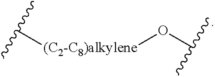

In some embodiments, the polyalkylene glycol unit comprises 2, 3, or 4 alkylene glycol subunits having the structure:

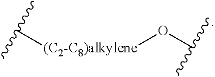

The polyalkylene glycol unit may comprise 2 or 3, in particular 2, alkylene glycol subunits having the structure:

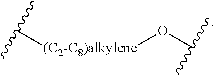

In some embodiments, the polyalkylene glycol unit comprises 10, 11, 12, 13 or 14 alkylene glycol subunits having the structure:

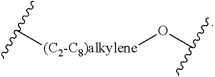

The polyalkylene glycol unit may comprise 11, 12 or 13, in particular 12, alkylene glycol subunits having the structure:

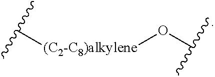

In some embodiments, the polyalkylene glycol unit comprises 22, 23, 24, 25 or 26 alkylene glycol subunits having the structure

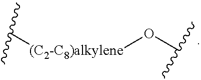

The polyalkylene glycol unit may comprise 23, 24 or 25, in particular 24, alkylene glycol subunits having the structure

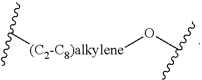

Preferably, the polyalkylene glycol unit used as R⁸⁰ may be a polyalkylene glycol unit comprising of from 1 to 40, preferably of from 1 to 32, more preferably of from 2 to 28 subunits having the structure:

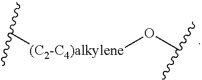

In some embodiments, the polyalkylene glycol unit comprises 2, 3, or 4 alkylene glycol subunits having the structure:

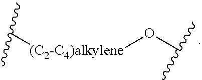

The polyalkylene glycol unit may comprise 2 or 3, in particular 2, alkylene glycol subunits having the structure:

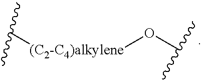

In some embodiments, the polyalkylene glycol unit comprises 10, 11, 12, 13 or 14 alkylene glycol subunits having the structure:

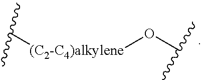

The polyalkylene glycol unit may comprise 11, 12 or 13, in particular 12, alkylene glycol subunits having the structure:

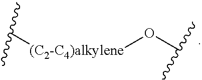

In some embodiments, the polyalkylene glycol unit comprises 22, 23, 24, 25 or 26 alkylene glycol subunits having the structure

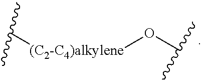

The polyalkylene glycol unit may comprise 23, 24 or 25, in particular 24, alkylene glycol subunits having the structure

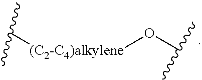

More preferably, the first polyalkylene glycol unit used as R⁸⁰ may be a polyalkylene glycol unit comprising of from 1 to 40, preferably of from 1 to 32, more preferably of from 2 to 28 subunits having the structure:

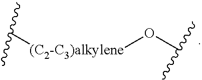

In some embodiments, the polyalkylene glycol unit comprises 2, 3, or 4 alkylene glycol subunits having the structure:

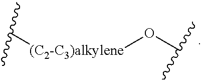

The polyalkylene glycol unit may comprise 2 or 3, in particular 2, alkylene glycol subunits having the structure:

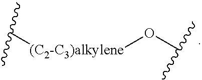

In some embodiments, the polyalkylene glycol unit comprises 10, 11, 12, 13 or 14 alkylene glycol subunits having the structure:

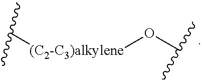

The polyalkylene glycol unit may comprise 11, 12 or 13, in particular 12, alkylene glycol subunits having the structure:

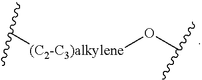

In some embodiments, the polyalkylene glycol unit comprises 22, 23, 24, 25 or 26 alkylene glycol subunits having the structure

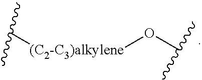

The polyalkylene glycol unit may comprise 23, 24 or 25, in particular 24, alkylene glycol subunits having the structure

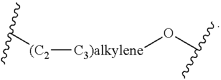

In very preferred embodiments, the polyalkylene glycol unit used as R⁸⁰ may be a polyethylene glycol unit comprising of from 1 to 40, preferably of from 1 to 32, more preferably of from 2 to 28 subunits each having the structure:

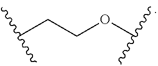 [SMILES: *CCO*]

In some embodiments, the polyalkylene glycol unit comprises 2, 3, or 4 subunits having the structure:

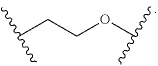 [SMILES: *CCO*]

The polyalkylene glycol unit may comprise 2 or 3, in particular 2, subunits having the structure:

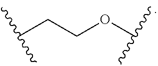 [SMILES: *CCO*]

In some embodiments, the polyalkylene glycol unit comprises 10, 11, 12, 13 or 14 alkylene glycol subunits having the structure:

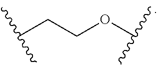 [SMILES: *CCO*]

The polyalkylene glycol unit may comprise 11, 12 or 13, in particular 12, alkylene glycol subunits having the structure:

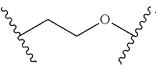 [SMILES: *CCO*]

In some embodiments, the polyalkylene glycol unit comprises 22, 23, 24, 25 or 26 alkylene glycol subunits having the structure

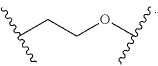 [SMILES: *CCO*]

The polyalkylene glycol unit may comprise 23, 24 or 25, in particular 24, alkylene glycol subunits having the structure

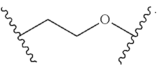 [SMILES: *CCO*]

Preferably, the polyalkylene glycol unit used as R^{C50} is:

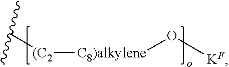

wherein:
[symbol] indicates the position of the O attached to the phosphorus;
K^{F} is H or a capping group; preferably K^{F} is selected from the group consisting of —H (hydrogen), —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₈)alkyl and —(C₂-C₁₀)alkyl-N((C₁-C₈)alkyl)₂; more preferably K^{F} is H; and
o is an integer ranging from 1 to 100.

The “capping group”, when referred to herein, may be any moiety which is capable to function as a terminal group of the polyalkylene glycol unit. Examples for first capping groups, which can be used in the present disclosure, include —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₈)alkyl and —(C₂-C₁₀)alkyl-N((C₁-C₈)alkyl)₂. In some embodiments, the capping group may be —(C₁-C₁₀)alkyl, in particular methyl.

Preferably, K^{F} is H (hydrogen).

The integer o denotes the number of repeating units

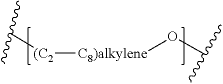

in the polyalkylene glycol unit. The integer o may range from 1 to 100. Preferably, o ranges from 2 to 50. More preferably, o ranges from 3 to 45. Still more preferably, o ranges from 4 to 40. Still more preferably, o ranges from 6 to 35. Even more preferably, o ranges from 8 to 30. In preferred embodiments, o is 12 or about 12. In preferred embodiments, o is 24 or about 24. Preferably, the repeating unit is

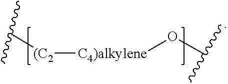

More preferably, the repeating unit is

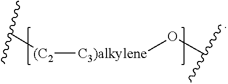

In the polyalkylene glycol unit, the integer o may range from 1 to 40. Preferably, o ranges from 1 to 32. More preferably, o ranges from 2 to 28. In some embodiments, the integer o is 2, 3 or 4. The integer o may be 2 or 3, in particular 2. In some embodiments, the integer o is 10, 11, 12, 13 or 14. The integer o may be 11, 12 or 13, in particular 12. In some embodiments, the integer o is 22, 23, 24, 25 or 26. The integer o may be 23, 24 or 25, in particular 24. Preferably, the repeating unit is

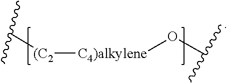

More preferably, the repeating unit is

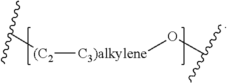

Preferably, the polyalkylene glycol unit used as R⁸⁰ comprises ethylene glycol subunits each having the following structure:

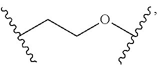 [SMILES: *CCO*]

i.e. this subunit is denoted an “ethylene glycol subunit”. Accordingly, preferably the polyalkylene glycol unit used as R⁸⁰ is a polyethylene glycol unit. The polyethylene glycol unit comprises at least one ethylene glycol subunit.

Preferably, the polyalkylene glycol unit used as R⁸⁰ may be a polyethylene glycol unit comprising of from 1 to 100, preferably of from 2 to 50, more preferably of from 3 to 45, still more preferably of from 4 to 40, still more preferably of from 6 to 35, even more preferably of from 8 to 30 ethylene glycol subunits each having the structure:

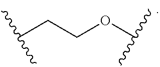 [SMILES: *CCO*]

Preferably, the polyalkylene glycol unit used as R⁸⁰ may be a polyethylene glycol unit comprising of from 1 to 40, preferably of from 1 to 32, more preferably of from 2 to 28 ethylene glycol subunits each having the structure:

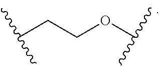 [SMILES: *CCO*]

In some embodiments, the polyethylene glycol unit comprises 2, 3, or 4 ethylene glycol subunits each having the structure:

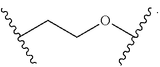 [SMILES: *CCO*]

The polyethylene glycol unit may comprise 2 or 3, in particular 2, ethylene glycol subunits each having the structure:

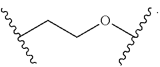 [SMILES: *CCO*]

In some embodiments, the polyethylene glycol unit comprises 10, 11, 12, 13 or 14 ethylene glycol subunits each having the structure:

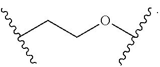 [SMILES: *CCO*]

The polyethylene glycol unit may comprise 11, 12 or 13, in particular 12, ethylene glycol subunits each having the structure:

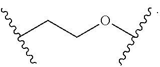 [SMILES: *CCO*]

In some embodiments, the polyethylene glycol unit comprises 22, 23, 24, 25 or 26 ethylene glycol subunits each having the structure:

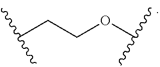 [SMILES: *CCO*]

The polyethylene glycol unit may comprise 23, 24 or 25, in particular 24, ethylene glycol subunits each having the structure:

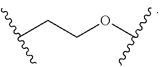 [SMILES: *CCO*]

Preferably, the polyalkylene glycol unit used as R⁸⁰ is a polyethylene glycol unit having the structure:

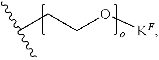

wherein:
[symbol] indicates the position of the O attached to the phosphorus;
K^{F} is H (hydrogen) or a first capping group as described herein; preferably K^{F} is selected from the group consisting of —H (hydrogen), —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₈)alkyl and —(C₂-C₁₀)alkyl-N((C₁-C₈)alkyl)₂; more preferably K^{F} is H; and
o is an integer ranging from 1 to 100.

The integer o denotes the number of repeating units

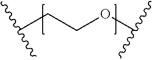

in the polyethylene glycol unit. The integer o may range from 1 to 100. Preferably, o ranges from 2 to 50. More preferably, o ranges from 3 to 45. Still more preferably, o ranges from 4 to 40. Still more preferably, o ranges from 6 to 35. Even more preferably, o ranges from 8 to 30. In preferred embodiments, o is 12 or about 12. In preferred embodiments, o is 24 or about 24.

In the polyethylene glycol unit used as R⁸⁰, the integer o may range from 1 to 40. Preferably, o ranges from 1 to 32. More preferably, o ranges from 2 to 28. In some embodiments, the integer o is 2, 3 or 4. The integer o may be 2 or 3, in particular 2. In some embodiments, the integer o is 10, 11, 12, 13 or 14. The integer o may be 11, 12 or 13, in particular 12. In some embodiments, the integer o is 22, 23, 24, 25 or 26. The integer o may be 23, 24 or 25, in particular 24.

In general, in the polyalkylene glycol unit used as R⁸⁰, (preferably, polyethylene glycol unit), polydisperse polyalkylene glycols (preferably, polydisperse polyethylene glycols), monodisperse polyalkylene glycols (preferably, monodisperse polyethylene glycol), and discrete polyalkylene glycols (preferably, discrete polyethylene glycols) can be used. Polydisperse polyalkylene glycols (preferably, polydisperse polyethylene glycols) are a heterogenous mixture of sizes and molecular weights, whereas monodisperse polyalkylene glycols (preferably, monodisperse polyethylene glycols) are typically purified from heterogenous mixtures and therefore provide a single chain length and molecular weight. Preferred polyalkylene glycols units are discrete polyalkylene glycols (preferably, discrete polyethylene glycols), i.e. compounds that are synthesized in step-wise fashion and not via a polymerization process. Discrete polyalkylene glycols (preferably, discrete polyethylene glycols) provide a single molecule with defined and specified chain length.

The polyalkylene glycol unit (preferably, polyethylene glycol unit) provided herein and used as R⁸⁰ may comprise one or multiple polyalkylene glycol chains (preferably, polyethylene glycol chains). The polyalkylene glycol chains (preferably, polyethylene glycol chains) can be linked together, for example, in a linear, branched or star shaped configuration. Optionally, at least one of the polyalkylene glycol chains (preferably, polyethylene glycol chains) may be derivatized at one end for covalent attachment to the oxygen atom bound to the phosphorus.

The polyalkylene glycol unit (preferably, polyethylene glycol unit) used as R⁸⁰ will be attached to the conjugate (or intermediate thereof) at the oxygen atom which is bound to the phosphorus. The other terminus (or termini) of the polyalkylene glycol unit (preferably, polyethylene glycol unit) will be free and untethered and may take the form of a hydrogen, methoxy, carboxylic acid, alcohol or other suitable functional group, such as e.g. any capping group as described herein. The methoxy, carboxylic acid, alcohol or other suitable functional group acts as a cap for the terminal polyalkylene glycol subunit (preferably, polyethylene glycol subunit) of the polyalkylene glycol unit (preferably, polyethylene glycol unit). By untethered, it is meant that the polyalkylene glycol unit (preferably, polyethylene glycol unit) will not be attached at that untethered site to, e.g., a drug moiety (D), to a receptor binding molecule (RBM), or to a component of the linker (L) linking a drug moiety and/or a receptor binding molecule. For those embodiments wherein the polyalkylene glycol unit (preferably, polyethylene glycol unit) comprises more than one polyalkylene glycol chain (preferably, polyethylene glycol chain), the multiple polyalkylene glycol chains (preferably, polyethylene glycol chains) may be the same or different chemical moieties (e.g., polyalkylene glycols, in particular polyethylene glycols, of different molecular weight or number of subunits). The multiple first polyalkylene glycol chains (preferably, first polyethylene glycol chains) are attached to the oxygen atom bound to the phosphorus at a single attachment site. The skilled artisan will understand that the polyalkylene glycol unit (preferably, polyethylene glycol unit) in addition to comprising repeating polyalkylene glycol subunits (preferably, polyethylene glycol subunits) may also contain non-polyalkylene glycol material (preferably, non-polyethylene glycol material) (e.g., to facilitate coupling of multiple polyalkylene glycol chains (preferably, polyethylene glycol chains) to each other or to facilitate coupling to the oxygen atom bound to the phosphorus. Non-polyalkylene glycol material (preferably, non-polyethelyne glycol material) refers to the atoms in the polyalkylene glycol unit (preferably, first polyethylene glycol unit) that are not part of the repeating alkylene glycol subunits (preferably, —CH₂CH₂O— subunits). In embodiments provided herein, the polyalkyleneglycol unit (preferably, polyethyleneglycol unit) can comprise two monomeric polyalkylene glycol chains (preferably, polyethylene glycol chains) linked to each other via non-polyalkylene glycol (non-polyethylene glycol) elements. In other embodiments provided herein, the polyalkylene glycol unit (preferably, polyethylene glycol unit) can comprise two linear polyalkylene glycol chains (preferably, polyethylene glycol chains) attached to a central core that is attached to the oxygen atom bound to the phosphorus (i.e., the polyalkylene glycol unit (preferably, polyethyleneglycol unit) is branched).

There are a number of polyalkylene glycol (preferably, polyethylene glycol) attachment methods available to those skilled in the art, [see, e.g., EP 0 401 384 (coupling PEG to G-CSF); U.S. Pat. No. 5,757,078 (PEGylation of EPO peptides); U.S. Pat. No. 5,672,662 (Polyethylene glycol) and related polymers mono substituted with propionic or butanoic acids and functional derivatives thereof for biotechnical applications); U.S. Pat. No. 6,077,939 (PEGylation of an N—terminal .alpha.-carbon of a peptide); and Veronese (2001) Biomaterials 22:405-417 (Review article on peptide and protein PEGylation)].

In preferred embodiments, the polyalkylene glycol unit, more preferably the polyethylene glycol unit, used as R⁸⁰ is directly attached to the oxygen atom bound to the phosphorus. In these embodiments, the polyalkylene glycol unit, preferably polyethylene glycol unit, does not comprise a functional group for attachment to the oxygen atom bound to the phosphorous, i.e. the oxygen atom is directly bound to a carbon atom of the polyalkylene glycol unit, preferably to a CH₂ of the polyethylene glycol unit.

In one group of embodiments, the polyalkylene glycol unit used as R⁸⁰ comprises at least 1 alkylene glycol subunit, preferably at least 2 alkylene glycol subunits, more preferably at least 3 alkylene glycol subunits, still more preferably at least 4 alkylene glycol subunits, still more preferably at least 6 alkylene glycol subunits, even more preferably at least 8 alkylene glycol subunits. In some such embodiments, the polyalkylene glycol unit comprises no more than about 100 alkylene glycol subunits, preferably no more than about 50 alkylene glycol units, more preferably no more than about 45 alkylene glycol subunits, more preferably no more than about 40 alkylene glycol subunits, more preferably no more than about 35 subunits, even more preferably no more than about 30 alkylene glycol subunits. In any one of these embodiments, the alkylene glycol subunit may be any alkylene glycol subunit as described herein. Preferably, in any one of these embodiments, each alkylene glycol subunit is an ethylene glycol subunit having the following structure:

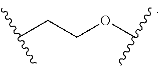 [SMILES: *CCO*]

Preferably, when each alkylene glycol subunit is an ethylene glycol subunit, in any one of these embodiments the polyalkylene glycol unit is a polyethylene glycol unit.

Preferably, in any one of these embodiments, each alkylene glycol subunit is an ethylene glycol subunit having the following structure:

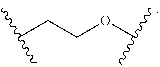 [SMILES: *CCO*]

Preferably, when each alkylene glycol subunit is an ethylene glycol subunit, in any one of these embodiments the polyalkylene glycol unit is a polyethylene glycol unit comprising one or more linear polyethylene glycol chains.

In another group of embodiments, the polyalkylene glycol unit used as R⁸⁰ comprises a combined total of from 1 to 100, preferably of from 2 to 50, more preferably of from 3 to 45, still more preferably of from 4 to 40, still more preferably of from 6 to 35, even more preferably of from 8 to 30 alkylene glycol subunits. In any one of these embodiments, the alkylene glycol subunit may be any alkylene glycol subunit as described herein. Preferably, in any one of these embodiments, each alkylene glycol subunit is an ethylene glycol subunit having the following structure:

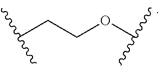 [SMILES: *CCO*]

Preferably, when each alkylene glycol subunit is an ethylene glycol subunit, in any one of these embodiments the polyalkylene glycol unit is a polyethylene glycol unit.

In another group of embodiments, the polyalkylene glycol unit used as R⁸⁰ comprises one or more linear polyalkylene glycol chains having a combined total of from 1 to 100, preferably 2 to 50, more preferably 3 to 45, still more preferably 4 to 40, still more preferably 6 to 35, even more preferably 8 to 30 alkylene glycol subunits. In any one of these embodiments, the alkylene glycol subunit may be any alkylene glycol subunit as described herein. Preferably, in any one of these embodiments, each alkylene glycol subunit is an ethylene glycol subunit having the following structure:

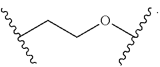 [SMILES: *CCO*]

Preferably, when each alkylene glycol subunit is an ethylene glycol subunit, in any one of these embodiments the polyalkylene glycol unit is a polyethylene glycol unit comprising one or more linear polyethylene glycol chains.

In another group of embodiments, the polyalkylene glycol unit used as R⁸⁰ is a linear single polyalkylene glycol chain having at least 1 subunit, preferably at least 2 subunits, more preferably at least 3 subunits, still more preferably at least 6 subunits, even more preferably at least 8 subunits. In any one of these embodiments, the alkylene glycol subunit may be any alkylene glycol subunit as described herein. Preferably, in any one of these embodiments, each alkylene glycol subunit is an ethylene glycol subunit having the following structure:

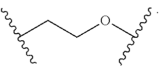 [SMILES: *CCO*]

Preferably, when each alkylene glycol subunit is an ethylene glycol subunit, in any one of these embodiments the polyalkylene glycol unit is a polyethylene glycol unit which is a linear single polyethylene glycol chain. Optionally, in any one of these embodiments the linear single polyalkylene glycol chain may be derivatized.

In another group of embodiments, the polyalkylene glycol unit used as R⁸⁰ is a linear single polyalkylene glycol chain having from 1 to 100, preferably 2 to 50, more preferably 3 to 45, more preferably 4 to 40, more preferably 6 to 35, more preferably 8 to 30 alkylene glycol subunits. In any one of these embodiments, the alkylene glycol subunit may be any alkylene glycol subunit as described herein. Preferably, in any one of these embodiments each alkylene glycol subunit is an ethylene glycol subunit having the following structure:

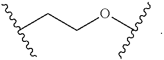 [SMILES: *CCO*]

Preferably, when each alkylene glycol subunit is an ethylene glycol subunit, in any one of these embodiments the polyalkylene glycol unit is a polyethylene glycol unit which is a linear single polyethylene glycol chain. Optionally, in any one of these embodiments the linear single polyalkylene glycol chain may be derivatized.

Exemplary linear polyethylene glycol units that can be used for R⁸⁰ as polyalkylene glycol unit, in particular as a polyethylene glycol unit, in any one of the embodiments provided herein are as follows:

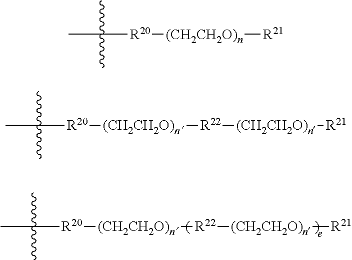

wherein the wavy line indicates the site of attachment to the oxygen atom bound to the phosphorus;
R²⁰ is a PEG attachment unit; preferably, R²⁰ is absent;
R²¹ is a PEG capping unit (herein, R²¹ is also denoted as “K^{F}”);
R²² is a PEG coupling unit (i.e. for coupling multiple PEG subunit chains together;
n is independently selected from 1 to 100, preferably from 2 to 50, more preferably from 3 to 45, more preferably from 4 to 40, still more preferably from 6 to 35, even more preferably from 8 to 30;
e is 2 to 5;
each n′ is independently selected from 1 to 100, preferably from 2 to 50, more preferably from 3 to 45, more preferably from 4 to 40, still more preferably from 6 to 35, even more preferably from 8 to 30. In preferred embodiments, there are at least 1, preferably at least 2, more preferably at least 3, more preferably at least 4, more preferably at least 6, even more preferably at least 8 ethylene glycol subunits in the polyethylene glycol unit. In some embodiments, there are no more than 100, preferably no more than 50, more preferably no more than 45, more preferably no more than 40, more preferably no more then 35, even more preferably no more than 30 ethylene glycol subunits in the polyethylene glycol unit. When R²⁰ is absent, a (CH₂CH₂O) subunit is directly bound to the oxygen atom, which is attached to the phosphorus.

Preferably, the linear polyethylene glycol unit is

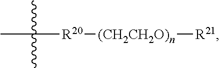

wherein the wavy line indicates the site of attachment to the oxygen atom bound to the phosphorus; R²⁰, R²¹ (also denoted herein as “K^{F}”) and n are as defined herein; more preferably R²⁰ is absent. In preferred embodiments, n is 12 or about 12. In preferred embodiments, n is 24 or about 24. Preferably, R²¹ is H.

The polyethylene glycol attachment unit R²⁰, when present, is part of the polyethylene glycol unit and acts to link the polyethylene glycol unit used as R⁸⁰ to the oxygen atom bound to the phosphorus. In this regard, the oxygen atom bound to the phosphorus forms a bond with the first polyethylene glycol unit. In exemplary embodiments, the PEG attachment unit R²⁰, when present, is selected from the group consisting of *—(C₁-C₁₀)alkyl-^{#}, *-arylene-^{#}, *—(C₁-C₁₀)alkyl-O—^{#}, *—(C₁-C₁₀)alkyl-C(O)—^{#}, *—(C₁-C₁₀)alkyl-C(O)O—^{#}, *—(C₁-C₁₀)alkyl-NH-^{#}, *—(C₁-C₁₀)alkyl-S-^{#}, *—(C₁-C₁₀)alkyl-C(O)—NH-^{#}, *—(C₁-C₁₀)alkyl-NH—C(O)—^{#}, and *—CH₂—CH₂SO₂—(C₁-C₁₀)alkyl-^{#}; wherein * denotes the attachment to the oxygen bound to the phosphorus, and #denotes the attachment to the ethylene glycol unit.

The PEG coupling unit R²², when present, is part of the polyethylene glycol unit and is non-PEG material that acts to connect two or more chains of repeating —CH₂CH₂O— subunits. In exemplary embodiments, the PEG coupling unit R²², when present, is independently selected from the group consisting of *—(C₁-C₁₀)alkyl-C(O)—NH-^{#}, *—(C₁-C₁₀)alkyl-NH—C(O)—^{#}, *—(C₂-C₁₀)alkyl-NH-^{#}, *—(C₂-C₁₀)alkyl-O—^{#}, *—(C₁-C₁₀)alkyl-S—^{#}, or *—(C₂-C₁₀)alkyl-NH—^{#}; wherein * denotes the attachment to an oxygen atom of an ethylene glycol subunit, and #denotes the attachment to a carbon atom of another ethylene glycol subunit.

The group R²¹, also denoted herein as “K^{F}”, in exemplary embodiments is H (hydrogen), or may be a capping group, as described herein; preferably, R²¹ is independently selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₈)alkyl and —(C₂-C₁₀)alkyl-N((C₁-C₈)alkyl)₂. In some embodiments R²¹ may be —(C₁-C₁₀)alkyl, in particular methyl. More preferably, R²¹ is H.

Illustrative linear polyethylene glycol units, which can be used as polyalkylene glycol units for R⁸⁰ in any one of the embodiments provided herein, are as follows.

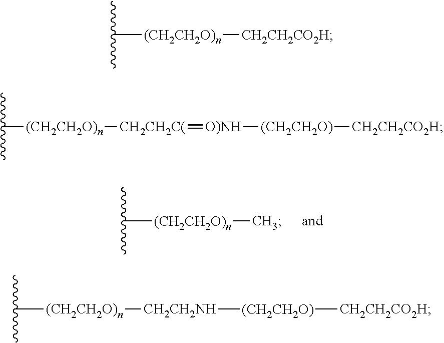

wherein the wavy line indicates the site of attachment to the oxygen atom which is bound to the phosphorus; and each n is from 1 to 100, preferably from 2 to 50, more preferably from 3 to 45, still more preferably from 4 to 40, still more preferably from 6 to 35, even more preferably from 8 to 30. In some embodiments, n is about 12. In some embodiments, n is about 24.

In some embodiments, the polyalkylene glycol unit used as R⁸⁰ is from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the polyalkylene glycol unit may have at least 6 alkylene glycol subunits or at least 8 alkylene glycol subunits. In some such aspects, the first polyalkylene glycol unit may have at least 6 alkylene glycol subunits or at least 8 alkylene glycol subunits but no more than 100 alkylene glycol subunits, preferably no more than 50 alkylene glycol subunits. In some embodiments, the polyalkylene glycol unit is a polyethylene glycol unit being from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the polyethylene glycol unit may have at least 6 ethylene glycol subunits or at least 8 ethylene glycol subunits. In some such aspects, the polyethylene glycol unit has at least 6 ethylene glycol subunits or at least 8 ethylene glycol subunits but no more than 100 ethylene glycol subunits, preferably no more than 50 ethylene glycol subunits.

It will be appreciated that when referring to alkylene glycol subunits, in particular ethylene glycol subunits, and depending on context, the number of subunits can represent an average number, e.g., when referring to a population of conjugates or intermediate compounds, and using polydisperse polyalkylene glycols, in particular polydisperse polyethylene glycols.

### Conjugate of Formula (Ia)

The present invention also relates to a conjugate having formula (Ia):

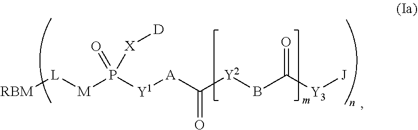

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and (C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

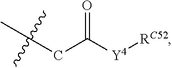

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkyl(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl; and
n is an integer ranging from 1 to 20.

Preferably, the conjugate of formula (Ia) has formula (Ia1):

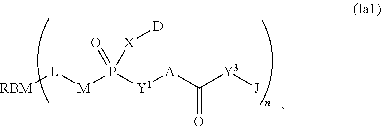

or a pharmaceutically acceptable salt or solvate thereof;
wherein RBM, L, M, X, D, Y¹, A, Y³, J and n are as defined herein.

More preferably, the conjugate of formula (Ia1) has formula (Ia2):

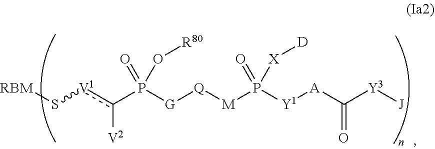

or a pharmaceutically acceptable salt or solvate thereof;
wherein RBM, V¹, V², [symbol] , R⁸⁰, G, Q, M, X, D, Y¹, A, Y³, J and n are as defined herein.

In a conjugate of formula (Ia), (Ia1) or (Ia2) any variable, such as, for example, RBM, L, V¹, V², [symbol] , R⁸⁰, G, Q, M, X, D, Y¹, A, Y², B, m, Y³, J and n, may be as defined herein.

### Conjugate of Formula (Ib)

The present invention also relates to a conjugate having the formula (Ib)

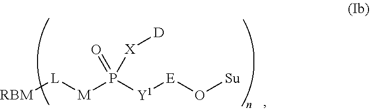

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Su is a sugar moiety which is bound to the oxygen atom (O) via a cleavable bond; and
wherein the oxygen, after cleavage of the sugar moiety Su, is capable of forming a ring, preferably a four- to seven-membered ring, more preferably a five- or six-membered ring, together with the spacer E, Y¹ and the phosphorus; and
n is an integer ranging from 1 to 20.

In some preferred embodiments, the formula (Ib) has the structure of formula (Ib1):

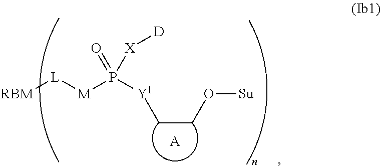

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;

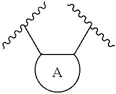

is an optionally substituted four- to seven-membered, preferably five- or six-membered, carbocyclic or heterocyclic ring;
[symbol] indicate the positions of the oxygen atom and Y¹; preferably the attachment points to the oxygen atom and Y¹ are two adjacent atoms of the ring;
Su is a sugar moiety; and
n is an integer ranging from 1 to 20. In particular, the bond between the oxygen (O) and the sugar moiety (Su) may be cleavable, such as, for example, by a glycosidase.

In a conjugate of formula (Ib1) any variable, such as, for example, RBM, L, M, X, D, Y¹,

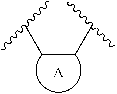

[symbol]

, Su and n, may be as defined herein.

In a conjugate of formula (Ib) or (Ib1), the sugar moiety is not particularly limited and can be any suitable glycoside, including glycosides that are modified with suitable protecting groups at the hydroxyl functionalities. Suitable protecting groups are generally known and include, for example, acyl esters such as acetyl ester, lactic acid esters, ethers, sulfate groups or phosphate moieties. The protecting groups at each hydroxy function of the sugar moiety may be the same or different from each other. The sugar moiety may be selected, for example, from the group consisting of glucuronic acid, galactose, glucose, arabinose, mannose-6-phosphate, fucose, rhamnose, gulose, allose, 6-deoxy-glucose, lactose, maltose, cellobiose, gentiobiose, maltotriose, GlcNAc, GalNAc and maltohexaose with and without protecting groups at the hydroxyl functionalities.

In some generally preferred embodiments, the sugar moiety is a sugar moiety that is modified with suitable protecting groups at the hydroxyl functionalities.

Preferably, each hydroxy function of the sugar moiety Su is protected with an acetyl ester.

### Conjugate of Formula (Ic1)

The present invention also relates to a conjugate having the formula (Ic1):

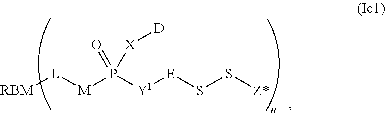

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

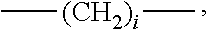

which can be optionally substituted; and wherein i is an integer ranging from 1 to 4; preferably 2, 3 or 4; more preferably 2 or 3; still more preferably 2,
Z* is an optionally substituted aliphatic residue or an optionally substituted aromatic residue; and
n is an integer ranging from 1 to 20.

In a conjugate of formula (Ic1) any variable, such as, for example, RBM, L, M, X, D, Y¹, E, i, Z* and n, may be as defined herein.

### Conjugate of Formula (Id1)

The present invention also relates to a conjugate of formula (Id1):

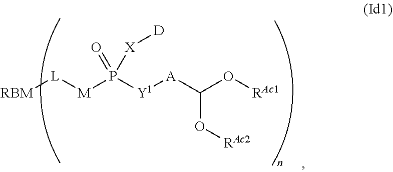

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
R^{Ac1} and R^{Ac2} are each independently an optionally substituted aliphatic residue or an optionally substituted aromatic residue; optionally, R^{Ac1} and R^{Ac2} can together with the oxygen atoms and the carbon atom form a 3- to 8-membered ring; and
n is an integer ranging from 1 to 20.

In a conjugate of formula (Id1), R^{Ac1} and R^{Ac2} are each independently an optionally substituted aliphatic residue or an optionally substituted aromatic residue. In some embodiments, R^{Ac1} and R^{Ac2} are each independently optionally substituted (C₁-C₈)alkyl. In some embodiments, R^{Ac1} and R^{Ac2} are each independently methyl, ethyl, propyl (such as, e.g., iso-propyl) or butyl (such as, e.g., tert-butyl). In any one of these embodiments R^{Ac1} and R^{Ac2} may be same or different; preferably, R^{Ac1} and R^{Ac2} are the same. Optionally, R^{Ac1} and R^{Ac2} can together with the oxygen atoms and the carbon atom form a 3- to 8-membered ring.

In a conjugate of formula (Id1) any variable, such as, for example, RBM, L, M, X, D, Y¹, A, R^{Ac1}, R^{Ac2}, and n, may be as defined herein. In particular, the group A may be as defined herein with regard to conjugates of formula (Ia).

### Conjugate of Formula (Ie1)

The present invention also relates to a conjugate of formula (Ie1):

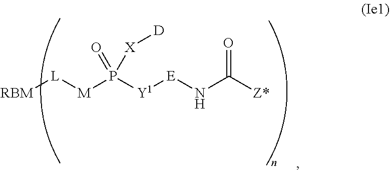

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

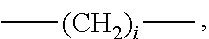

which can be optionally substituted; and wherein i is an integer ranging from 1 to 4; preferably 2, 3 or 4; more preferably 2 or 3; still more preferably 2;
Z* is an optionally substituted aliphatic residue or an optionally substituted aromatic residue;
wherein the nitrogen atom bound to the spacer E, after cleavage of the acetyl bond, is capable of forming a ring, preferably a four- to seven-membered ring, more preferably a five-or six-membered ring, together with the spacer E, Y¹ and the phosphorus; and
n is an integer ranging from 1 to 20.

In a conjugate of formula (Ie1), any variable, such as, for example, RBM, L, M, X, D, Y¹, E, i, Z* and n, may be as defined herein.

### Conjugate of Formula (If1)

The present invention also relates to a conjugate of formula (If1):

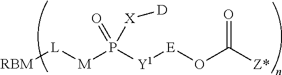

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

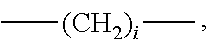

which can be optionally substituted; and wherein i is an integer ranging from 1 to 4; preferably 2, 3 or 4; more preferably 2 or 3; still more preferably 2;
Z* is an optionally substituted aliphatic residue or an optionally substituted aromatic residue;
wherein the nitrogen atom bound to the spacer E, after cleavage of the acetyl bond, is capable of forming a ring, preferably a four- to seven-membered ring, more preferably a five-or six-membered ring, together with the spacer E, Y¹ and the phosphorus; and
n is an integer ranging from 1 to 20.

In a conjugate of formula (Ie1), any variable, such as, for example, RBM, L, M, X, D, Y¹, E, i, Z* and n, may be as defined herein.

### Compound of Formula (II)

The present invention also relates to a compound having the formula (II):

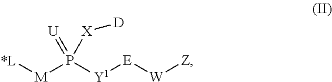

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group; and
W is a moiety which, after cleavage of the group Z, is capable of forming a ring, preferably a four- to seven-membered ring, more preferably a five- or six-membered ring, together with the spacer E, Y¹ and the phosphorus atom.

Preferably, the compound of formula (II) has the following formula:

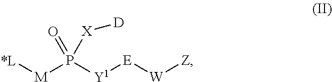

wherein L*, M, X, D, Y¹, E, W and Z are as defined herein.

In a compound of formula (II) any variable, such as, for example, L*, M, X, D, Y¹, E, W and Z, may be as defined herein.

The variable L* denotes a linker which is capable of forming a covalent attachment to a receptor binding molecule. Accordingly, a compound of formula (II) represents a precursor or intermediate for the preparation of a conjugate via reaction with a receptor binding molecule (RBM). In this regard, the linker L* has a functional group that is capable to form a covalent bond with a functional group of a receptor binding molecule (RBM). Useful functional groups that can be present on a receptor binding molecule, either naturally or via chemical manipulation, include but are not limited to, sulfhydryl (—SH), amino, hydroxyl, carboxy, the anomeric hydroxyl group of a carbohydrate, and carbonyl. Preferred functional groups of the receptor binding molecule are sulfhydryl and amino. The linker L* can comprise, for example, a maleimide group, an aldehyde, a ketone, a carbonyl, or a haloacetamide for attachment to the receptor binding molecule (RBM).

The linker L* of a compound of formula (II) may have an electrophilic group that is reactive to a nucleophilic group present on a receptor binding molecule (e.g., on an antibody). Useful nucleophilic groups on a receptor binding molecule include but are not limited to, sulfhydryl, hydroxyl and amino groups. A heteroatom of the nucleophilic group of a receptor binding molecule is reactive to an electrophilic group on a linker L* of compound of formula (II) and forms a covalent bond to the linker L*. Useful electrophilic groups of a linker L* include, but are not limited to, maleimide and haloacetamide groups. Accordingly, when the receptor binding molecule is, for example, an antibody, the electrophilic group of the linker L* provides a site for attachment of an antibody having a nucleophilic group.

In other embodiments, the linker L* of a compound of formula (II) may have a nucleophilic group that is reactive to an electrophilic group present on a receptor binding molecule (e.g., on an antibody). Useful electrophilic groups on a receptor binding molecule include, but are not limited to, aldehyde and ketone and other carbonyl groups. A heteroatom of a nucleophilic group of a linker L* can react with an electrophilic group on a receptor binding molecule and form a covalent bond to the receptor binding molecule. Useful nucleophilic groups on a Linker L* include, but are not limited to, hydrazide, hydroxylamine, amino, hydrazine, thiosemicarbazone, hydrazine carboxylate, and arylhydrazide. Accordingly, when the receptor binding molecule is, for example, an antibody, the electrophilic group of the antibody provides a site for attachment to a linker L* having a nucleophilic group.

In some embodiments, the linker L* of a compound of formula (II) is capable to form a bond with a sulfur atom of the receptor binding molecule. The sulfur atom can be derived from a sulfhydryl group of a receptor binding molecule. Preferably, the receptor binding molecule is an antibody. Representative linkers L* which are capable to bind to a receptor binding molecule (RBM) are depicted in formulas (IIIa*) and (IIIb*), wherein Q is a connector unit and # indicates the attachment to the group M. Such linkers are described, e.g., in WO 2004/010957. The connector unit Q may be any connector unit Q as defined herein.

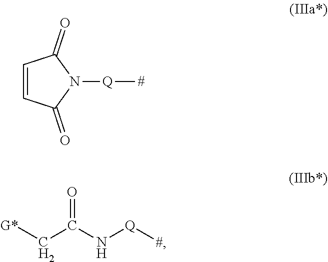

wherein G* is a leaving group; preferably G* is selected from the group consisting of Cl, Br, I, O-mesyl and O-tosyl.

In some embodiments, the linker L* of a compound of formula (II) contains a functional group that is capable to form a bond with a primary or secondary amino group of a receptor binding molecule. Examples of such functional groups include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative linkers bound to a receptor binding molecule (RBM) are depicted in formulas (Va*), (Vb*) and (Vc*), wherein Q is a connector unit and # indicates the attachment to the group M. Such linkers are described, e.g., in WO 2004/010957. The connector unit Q may be any connector unit Q as defined herein.

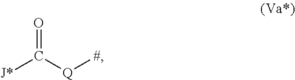

wherein J* is selected from the group consisting of Cl, Br, I, F, OH, —O—N-succinimide, —O-(4-nitrophenyl), —O-pentafluorophenyl, —O-tetrafluorophenyl and —O—C(O)—OR¹⁸, wherein R¹⁸ is (C₁-C₈)alkyl or aryl;

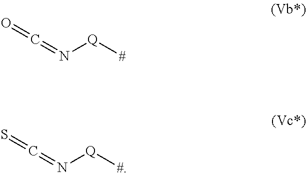

In some embodiments, the linker L* of a compound of formula (II) contains a functional group that can form a bond with an aldehyde group (denoted herein as —CHO or

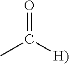

of a carbohydrate that can be present on the receptor binding molecule. For example, a carbohydrate can be mildly oxidized using a reagent such as sodium periodate, and the resulting —CHO group of the oxidized carbohydrate can be condensed with a linker that contains a functional group such as e.g. a hydrazide, an oxime, a primary or secondary amine, a hydrazine, a thiosemicarbazone, a hydrazine carboxylate, and an arylhydrazide such as those described by Kaneko, T. et al. Bioconjugate Chem. 1991, 2, 133-41. Representative linkers bound to a receptor binding molecule (RBM) are depicted in formulas (VIa), (VIb) and (VIc), wherein Q is a connector unit and # indicates the attachment to the group M. Such linkers are described, e.g., in WO 2004/010957. The connector unit Q may be any connector unit Q as defined herein.

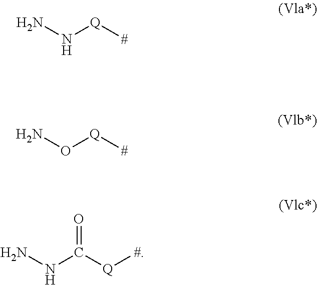

In preferred embodiments, the linker L* of a compound of formula (II) is:

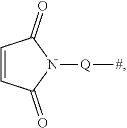

wherein Q is as defined herein, and # indicates the attachment to the group M. A linker L* having the structure

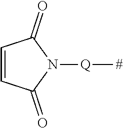

is capable to react with a thiol group (SH) of a receptor binding molecule, e.g. a thiol group of an antibody obtained by reduction of a disulfide bond. The linker L* is thus capable to form a covalent bond with the receptor binding molecule (RBM) via reaction of the thiol group with the double bond of the maleimide moiety to form a conjugate having the structure:

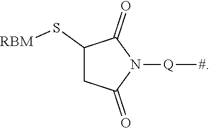

The connector unit Q may be any connector unit Q as defined herein. In particular, Q may be any connector unit Q as defined herein within the context of the linker L having the structure:

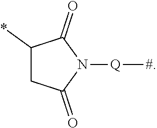

In some embodiments, the linker L* of a compound of formula (II) is:

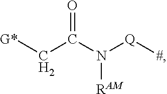

wherein:
G* is a leaving group; preferably G* is selected from the group consisting of Cl, Br, I, O— mesyl and O-tosyl; preferably G* is I;
R^{AM} is as defined herein;
Q is as defined herein; and
# indicates the attachment to the group M. A linker L* having the structure

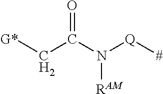

is capable to react with a thiol group (SH) of a receptor binding molecule, e.g. a thiol group of an antibody obtained by reduction of a disulfide bond. The linker L* is thus capable to form a covalent bond with the receptor binding molecule (RBM) via reaction of the thiol group, in particular a nucleophilic substitution at the carbon atom to which G* is bound, to replace G* and to form a conjugate having the structure:

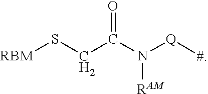

The connector unit Q may be any connector unit Q as defined herein. In particular, Q may be any connector unit Q as defined herein within the context of the linker L having the structure:

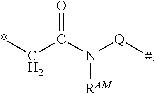

In preferred embodiments, the linker L* of a compound of formula (II) has the

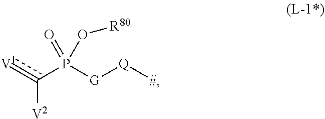

wherein:
[symbol] is a triple bond; or
[symbol] is a double bond;
V² is absent when [symbol] is a triple bond; or
V² is selected from the group consisting of hydrogen, (C₁-C₈)alkyl, (C₆-C₁₀)aryl, and (C₁-C₈)alkylene(C₆-C₁₀)aryl when [symbol] is a double bond;
V¹ is R^{V11—C} when [symbol] is a triple bond; or
V¹ is

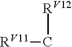

when [symbol] is a double bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl;
R^{C50} is an optionally substituted aliphatic residue or an optionally substituted aromatic residue; and
# indicates the attachment to the group M.

Phosphonamidate, phosphonothiolate and phosphonate moieties, which are comprised in the linker L-I*, and their preparations are generally known, e.g., from WO 2018/041985 and WO 2019/170710, which are hereby incorporated by reference in its entirety. A linker L-I* having the structure

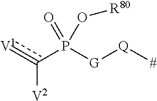

is capable to react with a thiol group (SH) of a receptor binding molecule, e.g. a thiol group of an antibody obtained by reduction of a disulfide bond. The linker L-I* is thus capable to a form a covalent bond with the receptor binding molecule (RBM) via reaction of the thiol group with the triple bond or double bond, respectively, to form a conjugate having the structure:

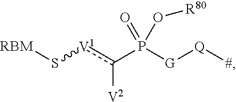

wherein,
when [symbol] in the linker L-I* of a compound of formula (II) is a triple bond, then [symbol] in the conjugate is a double bond; or
when [symbol] in the linker L-I* of a compound of formula (II) is a double bond, then [symbol] in the conjugate is a bond. Methods of preparing compounds of formula (II) having a linker L-I* are known in the art. As illustrative examples, compounds of formula (II) having a linker L-I*, wherein the group G is NH, may be prepared by using techniques and conditions, e.g. a Staudinger phosphonite reaction, as e.g. described in WO 2018/041985 A1, which is hereby incorporated by reference. Compounds of (II) having a linker L-I*, wherein the group G is S (sulfur) or O (oxygen), may be prepared by using techniques and conditions as e.g. described in WO 2019/170710, which is hereby incorporated by reference. In an analogous manner to the compounds of formula (II) having a linker L-I*, wherein G is S or O, as described in WO 2019/170710, compounds of formula (II) having a linker L-I*, wherein G is CR^{G71}R^{G72}, may be prepared, as illustrative examples, by substitution at the phosphorus atom using, e.g., a suitable organometallic compound, such as e.g. a Grignard compound or an organolithium compound. A person skilled in the art readily selects suitable methods and conditions to prepare compounds of formula (II) having a linker L-I*. The Examples section of the present disclosure also comprises guidance on how to prepare or obtain compounds of formula having a linker L-I* and/or conjugates of formula (I).

With regard to the representations [symbol] and [symbol] used herein, it is noted that, as commonly known to a person skilled in the art, each carbon atom is tetravalent. Accordingly, a structure

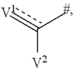

wherein V¹ and V² are as defined herein and # indicates attachment to the phosphorus, includes the structures

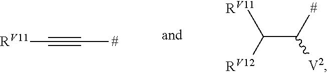

wherein R^{V11}, R^{V12} and V² are as defined herein. A structure

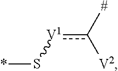

wherein V¹ and V² are as defined herein, # indicates attachment to the phosphorus and the asterisk (*) indicates attachment to the receptor binding molecule (RBM), includes the structures

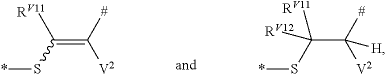

and wherein R^{V11}, R^{V12} and V² are as defined herein, and H is hydrogen. A wavy bond indicates that the configuration of the double bond may be E or Z. It is also possible that the compound is present as a mixture of the E and Z isomers.

In a linker of formula (L-I*) any variable, such as, for example, V¹, V², [symbol] , R⁸⁰, G and Q, may be as defined herein. In particular, in a linker of formula (L-I*) any variable, such as, for example, V¹, V², [symbol] , R⁸⁰, G and Q, may be as defined herein with regard to the linker (L-I).

Preferably R^{V11} is H or (C₁-C₈)alkyl; more preferably R^{V11} is H. Preferably R^{V12}, when present is H or (C₁-C₈)alkyl; more preferably R^{V12}, when present, is H. Preferably R^{G70}, when present is H or (C₁-C₈)alkyl; more preferably R^{G70}, when present, is H. Preferably R^{G71}, when present is H or (C₁-C₈)alkyl; more preferably R^{G71}, when present, is H. Preferably R^{G72}, when present is H or (C₁-C₈)alkyl; more preferably R^{G72}, when present, is H.

Preferably, [symbol] is a triple bond; V² is absent; V¹ is R^{V11—C}; and R^{V11} is selected from the group consisting of hydrogen, (C₁-C₈)alkyl, (C₆-C₁₀)aryl, and (C₁-C₈)alkylene(C₆-C₁₀)aryl; preferably R^{V11} is hydrogen or (C₁-C₈)alkyl; more preferably R^{V11} is hydrogen.

More preferably, [symbol] s a triple bond; V² is absent; V¹ is R_{V11-C}, and R^{V11} is H or (C₁-C₈)alkyl. Preferably, R^{V11} is H or (C₁-C₆)alkyl, more preferably H or (C₁-C₄)alkyl, still more preferably H or (C₁-C₂)alkyl. In preferred embodiments, R₃ is H.

In some embodiments, [symbol] is a double bond; V² is selected from the group consisting of hydrogen, (C₁-C₈)alkyl, (C₆-C₁₀)aryl, and (C₁-C₈)alkylene(C₆-C₁₀)aryl; preferably, V² is hydrogen or (C₁-C₈)alkyl; more preferably, V² is hydrogen; V¹ is

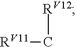

R^{V11} is selected from the group consisting of hydrogen, (C₁-C₈)alkyl, (C₆-C₁₀)aryl, and (C₁-C₈)alkylene(C₆-C₁₀)aryl; preferably R^{V11} is hydrogen or (C₁-C₈)alkyl, more preferably R^{V11} is hydrogen; R^{V12} is selected from the group consisting of hydrogen, (C₁-C₈)alkyl, (C₆-C₁₀)aryl, and (C₁-C₈)alkylene(C₆-C₁₀)aryl; preferably R^{V12} is hydrogen or (C₁-C₈)alkyl, more preferably R^{V12} is hydrogen.

In some embodiments, [symbol] is a double bond; V² may be H or (C₁-C₈)alkyl; V¹ is

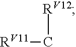

and R^{V11} and R^{V12} may independently be H or (C₁-C₈)alkyl. Preferably, R^{V11} and R^{V12} independently represent H or (C₁-C₆)alkyl, more preferably H or (C₁-C₄)alkyl, still more preferably H or (C₁-C₂)alkyl. Preferably, R^{V11} and R^{V12} are the same; even more preferably, R^{V11}, R^{V12} and V² are the same. More preferably, R^{V11} and R^{V12} are both H. Preferably, V² is H or (C₁-C₆)alkyl, more preferably H or (C₁-C₄)alkyl, still more preferably H or (C₁-C₂)alkyl. Even more preferably, V² is H. In preferred embodiments, R^{V11}, R^{V12} and V² are each H.

In some embodiments, the compound of formula (II) has formula (IIa):

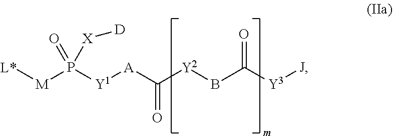

or a pharmaceutically acceptable salt or solvate thereof,
wherein:
L* is a linker capable of forming a covalent attachment to a receptor binding molecule (RBM);
M is O, NR^{M60}, or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
M is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

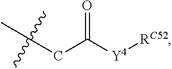

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl.

Preferably, the compound of formula (IIa) has formula (IIa1):

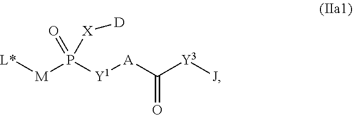

or a pharmaceutically acceptable salt or solvate thereof;
wherein L*, M, X, D, Y¹, A, Y³, J and n are as defined herein.

More preferably, the compound of formula (IIa) has formula (IIa2):

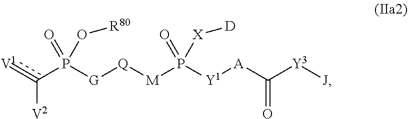

or a pharmaceutically acceptable salt or solvate thereof;
wherein V¹, V², [symbol] , R⁸⁰, G, Q, M, X, D, Y¹, A, Y³ and J are as defined herein.

In a compound of formula (IIa), (IIa1) or (IIa2), any variable, such as, for example, L*, V¹, V², [symbol] , R⁸⁰, G, Q, M, X, D, Y¹, A, Y², B, m, Y³ and J, may be as defined herein.

In some embodiments, the compound of formula (II) has formula (IIb):

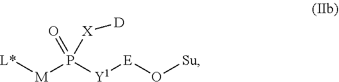

or a pharmaceutically acceptable salt or solvate thereof;
wherein:
L*, M, X, D and Y¹ are as defined herein;
E is a spacer;
Su is a sugar moiety which is bound to the oxygen atom via a cleavable bond; and
wherein the oxygen, after cleavage of the sugar moiety Su, is capable of forming a ring, preferably a four- to seven-membered ring, more preferably a five- or six-membered ring together with the spacer E, Y¹ and the phosphorus.

In the compound of formula (IIb), the sugar moiety is not particularly limited and can be any suitable glycoside, including glycosides that are modified with suitable protecting groups at the hydroxyl functionalities. Suitable protecting groups are generally known and include, for example, acyl esters such as acetyl ester, lactic acid esters, ethers, sulfate groups or phosphate moieties. The protecting groups at each hydroxy function of the sugar moiety may be the same or different from each other. The sugar moiety may be selected, for example, from the group consisting of glucuronic acid, galactose, glucose, arabinose, mannose-6-phosphate, fucose, rhamnose, gulose, allose, 6-deoxy-glucose, lactose, maltose, cellobiose, gentiobiose, maltotriose, GlcNAc, GaINAc and maltohexaose with and without protecting groups at the hydroxyl functionalities.

In some generally preferred embodiments, the sugar moiety is a sugar moiety that is modified with suitable protecting groups at the hydroxyl functionalities.

Preferably, each hydroxy function of the sugar moiety Su is protected with an acetyl ester.

Preferably, the compound of formula (IIb) has formula (IIb1):

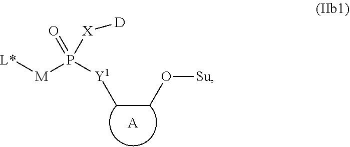

or a pharmaceutically acceptable salt or solvate thereof;
wherein L*, M, X, D, Y¹,

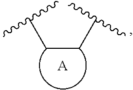

[symbol]

and Su are as defined herein.

In a compound of formula (IIb) or (IIb1) any variable, such as, for example, L*, M, X, D, Y¹,

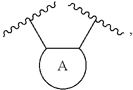

[symbol]

, E and Su, may be as defined herein.

In some embodiments, the compound of formula (II) has formula (IIc):

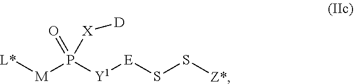

or a pharmaceutically acceptable salt or solvate thereof;
wherein:
L*, M, X, D and Y¹ are as defined herein;
E is a spacer;
Z* is an optionally substituted aliphatic residue or an optionally substituted aromatic residue; and
wherein the sulfur bound to the spacer E, after cleavage of the disulfide bond, is capable of forming a ring, preferably a four- to seven-membered ring, more preferably a five- or six-membered ring, together with the spacer E, Y¹ and the phosphorus atom.

Preferably, the compound of formula (IIc) has formula (IIc1):

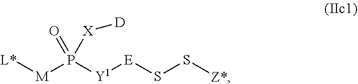

or a pharmaceutically acceptable salt or solvate thereof;
wherein:
L*, M, X, D, Y¹ and Z* are as defined herein; and
E is

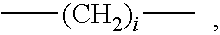

which can be optionally substituted; and wherein i is an integer ranging from 1 to 4; preferably 2, 3 or 4; more preferably 2 or 3; still more preferably 2.

In a compound of formula (IIc) or (IIc1) any variable, such as, for example, L*, M, X, D, Y¹, E, i and Z*, may be as defined herein.

In some embodiments, the compound of formula (II) has formula (IId):

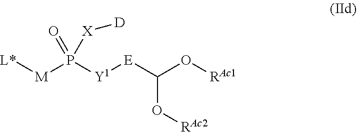

or a pharmaceutically acceptable salt or solvate thereof;
wherein:
L*, M, X, D, Y¹, E, R^{Ac1} and R^{Ac2} are as defined herein.

In some embodiments, the compound of formula (II) has formula (IId1):

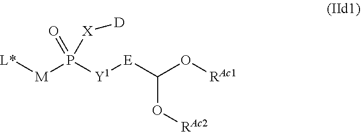

or a pharmaceutically acceptable salt or solvate thereof;
wherein:
L*, M, X, D, Y¹, A, R^{Ac1} and R^{Ac2} are as defined herein.

In some embodiments, the compound of formula (II) has formula (IIe):

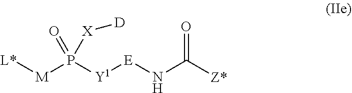

or a pharmaceutically acceptable salt or solvate thereof;
wherein:
L*, M, X, D, Y¹ and Z* are as defined herein; and
E is a spacer; preferably E is

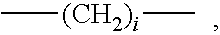

which can be optionally substituted; and wherein i is an integer ranging from 1 to 4; preferably 2, 3 or 4; more preferably 2 or 3; still more preferably 2,
wherein the nitrogen bound to the spacer E, after cleavage of the disulfide bond, is capable of forming a ring, preferably a four- to seven-membered ring, more preferably a five- or six-membered ring, together with the spacer E, Y¹ and the phosphorus atom.

In some embodiments, the compound of formula (II) has formula (IIf):

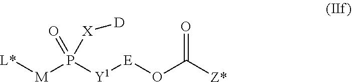

or a pharmaceutically acceptable salt or solvate thereof;
wherein:
L*, M, X, D, Y¹ and Z* are as defined herein; and
E is a spacer; preferably E is

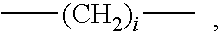

which can be optionally substituted; and wherein I is an integer ranging from 1 to 4; preferably 2, 3 or 4; more preferably 2 or 3; still more preferably 2,
wherein the oxygen bound to the spacer E, after cleavage of the disulfide bond, is capable of forming a ring, preferably a four- to seven-membered ring, more preferably a five- or six-membered ring, together with the spacer E, Y¹ and the phosphorus atom.

### Compound of Formula (IIa)

The present invention also relates to a compound having the formula (IIa):

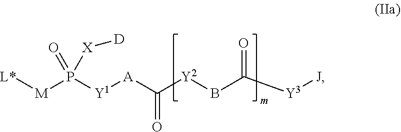

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

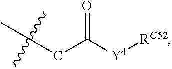

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkyl(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl.

Preferably, the compound of formula (IIa) has formula (IIa1):

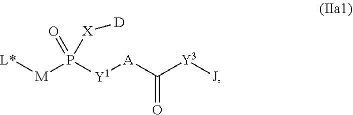

or a pharmaceutically acceptable salt or solvate thereof;
wherein L*, M, X, D, Y¹, A, Y³ and J are as defined herein.

More preferably, the compound of formula (IIa1) has formula (IIa2):

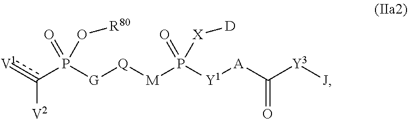

or a pharmaceutically acceptable salt or solvate thereof;
wherein V¹, V², [symbol] , R⁸⁰, G, Q, M, X, D, Y¹, A, Y³ and J are as defined herein.

In a compound of formula (IIa), (IIa1) or (IIa2) any variable, such as, for example, L*, V¹, V², [symbol] , R⁸⁰, G, Q, M, X, D, Y¹, A, Y², B, Y³, J and m, may be as defined herein.

### Compound of Formula (IIb1):

The present invention also relates to a compound having the formula (IIb1):

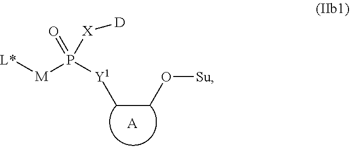

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;

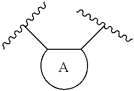

is an optionally substituted four- to seven-membered, preferably five- or six-membered, carbocyclic or heterocyclic ring; and
[symbol] indicate the positions of the oxygen atom and Y¹; preferably the attachment points to the oxygen atom and Y¹ are two adjacent atoms of the ring; and
Su is a sugar moiety.

In a compound of formula (IIb1) any variable, such as, for example, L*, M, X, D, Y¹,

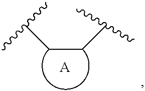

[symbol]

and Su may be as defined herein.

### Compound of Formula (IIc1):

The present invention also relates to a compound having the formula (IIc1):

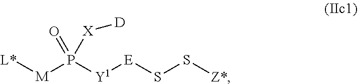

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

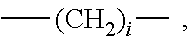

which can be optionally substituted; and wherein i is an integer ranging from 1 to 4; preferably 2, 3 or 4; more preferably 2 or 3; still more preferably 2; and
Z* is an optionally substituted aliphatic residue or an optionally substituted aromatic residue.

In a compound of formula (IIc1) any variable, such as, for example, L*, M, X, D, Y¹, E, i and Z*, may be as defined herein.

### Compound of Formula (IId1):

The present invention also relates to a compound having the formula (IId1):

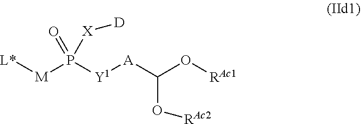

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring; and
R^{Ac1} and R^{Ac2} are each independently an optionally substituted aliphatic residue or an optionally substituted aromatic residue; optionally, R^{Ac1} and R^{Ac2} can together with the oxygen atoms and the carbon atom form a 3- to 8-membered ring.

### Compound of Formula (IIe):

The present invention also relates to a compound having the formula (IIe1):

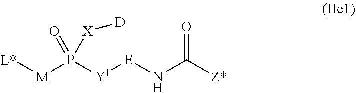

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

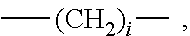

which can be optionally substituted; and wherein i is an integer ranging from 1 to 4; preferably 2, 3 or 4; more preferably 2 or 3; still more preferably 2; and
Z* is an optionally substituted aliphatic residue or an optionally substituted aromatic residue.

### Compound of Formula (IIf1):

The present invention also relates to a compound having the formula (IIf1):

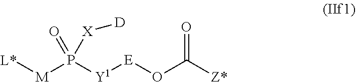

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

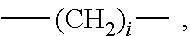

which can be optionally substituted; and wherein i is an integer ranging from 1 to 4; preferably 2, 3 or 4; more preferably 2 or 3; still more preferably 2; and
Z* is an optionally substituted aliphatic residue or an optionally substituted aromatic residue.

### Method of Preparing a Conjugate of Formula (I)

The present invention also relates to a method of preparing a conjugate of formula (I), said method comprising:
reacting a compound of formula (II)

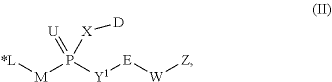

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group; and
W is a moiety which, after cleavage of the group Z, is capable of forming a ring, preferably a four- to seven-membered ring, more preferably a five- or six-membered ring, together with the spacer E, Y¹ and the phosphorus;
with a receptor binding molecule (RBM) having a functional group reactive towards L* of a compound of formula (II), thereby forming a covalent bond between the receptor binding molecule (RBM) and the linker (L) to result in a conjugate of formula (I):

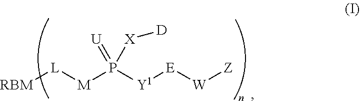

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group;
W is a moiety which, after cleavage of the group Z, is capable of forming a ring, preferably a four- to seven-membered ring, more preferably a five- or six-membered ring, together with the spacer E, Y¹ and the phosphorus; and
n is an integer ranging from 1 to 20.

Regarding the methods, in a compound of formula (II) and in a conjugate of formula (I) any variable, such as, for example, RBM, L*, L, M, X, D, Y¹, E, W, Z and n, may be as defined herein.

In some embodiments, the method of preparing a conjugate of formula (I) comprises:
Reacting a compound of formula (IIa):

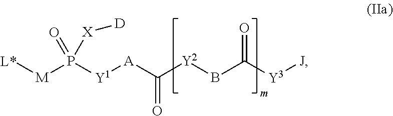

or a pharmaceutically acceptable salt or solvate thereof;
wherein:
L*, M, X, D and Y¹ are as defined herein;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₃)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

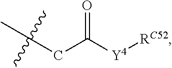

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37} wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C30} and R^{C31} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkyl(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl
with a receptor binding molecule (RBM) having a functional group reactive towards L* of a compound of formula (IIa), thereby forming a covalent bond between the receptor binding molecule (RBM) and the linker (L) to result in a conjugate of formula (Ia)

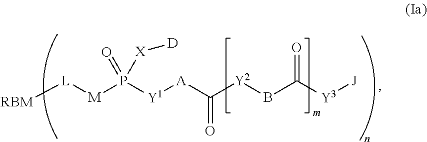

or a pharmaceutically acceptable salt or solvate thereof;
wherein:
RBM, L, M, X, D, Y¹ and n are as defined herein;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

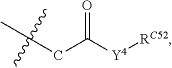

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C30} and R^{C31} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkyl(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl.

Preferably, the method of preparing a conjugate of formula (I) comprises:
Reacting a compound of formula (IIa1)

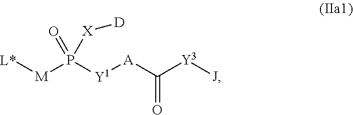

or a pharmaceutically active salt or solvate thereof;
wherein L*, M, X, D, Y¹, A, Y³ and J are as defined herein
with a receptor binding molecule (RBM) having a functional group reactive towards L* of a compound of formula (IIa1), thereby forming a covalent bond between the receptor binding molecule (RBM) and the linker (L) to result in a conjugate of formula (Ia1)

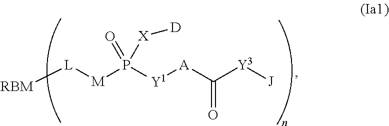

or a pharmaceutically acceptable salt or solvate thereof;
wherein RBM, L, M, X, D, Y¹, A, Y³, J and n are as defined herein.

More preferably, the method of preparing a conjugate of formula (I) comprises:
Reacting a compound of formula (IIa2):

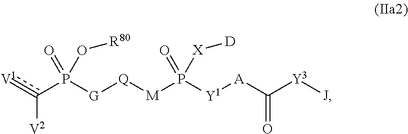

or a pharmaceutically acceptable salt or solvate thereof;
wherein M, X, D, Y¹, A, Y³ and J are as defined herein;
[symbol] is a triple bond; or
[symbol] is a double bond;
V² is absent when [symbol] is a triple bond; or
V² is selected from the group consisting of hydrogen, (C₁-C₈)alkyl, (C₆-C₁₀)aryl, and (C₁-C₈)alkylene(C₆-C₁₀)aryl when [symbol] is a double bond;
V¹ is R^{V11—C} when [symbol] is a triple bond; or
V¹ is

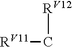

when [symbol] is a double bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl; and
R⁸⁰ is an optionally substituted aliphatic residue or an optionally substituted aromatic residue
with a thiol-containing molecule of formula (III)

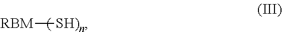

wherein RBM is a receptor binding molecule; and
n is an integer ranging from 1 to 20;
to result in a conjugate of formula (Ia2)

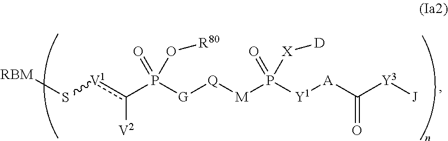

or a pharmaceutically acceptable salt or solvate thereof;
wherein:
RBM, M, X, D, Y¹, A, Y³, J and n are as defined herein;
[symbol] is a double bond when [symbol] in a compound of formula (IIa2) is a triple bond; or
[symbol] is a bond when [symbol] in a compound of formula (IIa2) is a double bond;
V² is absent when [symbol] is a double bond; or
V² is selected from the group consisting of hydrogen, (C₁-C₈)alkyl, (C₆-C₁₀)aryl, and (C₁-C₃)alkylene(C₅-C₁₀)aryl when [symbol] is a bond;
V¹ is R^{V11—C} when [symbol] is a double bond; or
V¹ is

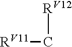

when [symbol] is a bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl; and
R⁸⁰ is an optionally substituted aliphatic residue or an optionally substituted aromatic residue.

Regarding the methods, in a compound of formula (IIa), (IIa1) or (IIa2) and a conjugate of formula (Ia), (Ia1) or (Ia2) any variable, such as, for example, RBM, L, L*, V¹, V², [symbol] , [symbol] , R⁸⁰, G, Q, M, X, D, Y¹, A, Y², B, Y³, J, m and n may be as defined herein.

In some embodiments, the method of preparing a conjugate of formula (I) comprises:
Reacting a compound of formula (IIb)

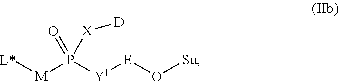

or a pharmaceutically acceptable salt or solvate thereof;
wherein:
L*, M, X, D and Y¹ are as defined herein;
E is a spacer;
Su is a sugar moiety which is bound to the oxygen atom via a cleavable bond; and
wherein the oxygen, after cleavage of the sugar moiety Su, is capable of forming a ring, preferably a four- to seven-membered ring, more preferably a five- or six-membered ring, together with the spacer E, Y¹ and the phosphorus;
with a receptor binding molecule (RBM) having a functional group reactive towards L* of a compound of formula (IIb), thereby forming a covalent bond between the receptor binding molecule (RBM) and the linker (L) to result in a conjugate of formula (Ib)

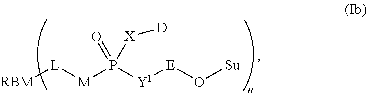

or a pharmaceutically acceptable salt or solvate thereof;
wherein:
RBM, L, M, X, D, Y¹ and n are as defined herein;
E is a spacer;
Su is a sugar moiety which is bound to the oxygen atom via a cleavable bond; and
wherein the oxygen, after cleavage of the sugar moiety Su, is capable of forming a ring, preferably a four- to seven-membered ring, more preferably a five- or six-membered ring, together with the spacer E, Y¹ and the phosphorus.

In the compound of formula (Ib) and (IIb), the sugar moiety is not particularly limited and can be any suitable glycoside, including glycosides that are modified with suitable protecting groups at the hydroxyl functionalities. Suitable protecting groups are generally known and include, for example, acyl esters such as acetyl ester, lactic acid esters, ethers, sulfate groups or phosphate moieties. The protecting groups at each hydroxy function of the sugar moiety may be the same or different from each other. The sugar moiety may be selected, for example, from the group consisting of glucuronic acid, galactose, glucose, arabinose, mannose-6-phosphate, fucose, rhamnose, gulose, allose, 6-deoxy-glucose, lactose, maltose, cellobiose, gentiobiose, maltotriose, GlcNAc, GaINAc and maltohexaose with and without protecting groups at the hydroxyl functionalities.

In some generally preferred embodiments, the sugar moiety is a sugar moiety that is modified with suitable protecting groups at the hydroxyl functionalities.

Preferably, each hydroxy function of the sugar moiety Su is protected with an acetyl ester.

In some embodiments the method of preparing a conjugate of formula (I) comprises:
Reacting a compound of formula (IIb1):

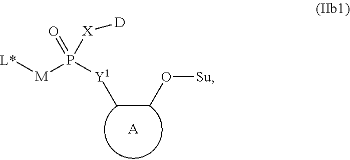

or a pharmaceutically acceptable salt or solvate thereof;
wherein:
L*, M, X, D, Y¹ and Su are as defined herein;

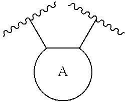

is an optionally substituted four- to seven-membered, preferably five- or six-membered, carbocyclic or heterocyclic ring; and
[symbol] indicate the positions of the oxygen atom and Y¹; preferably the attachment points to the oxygen atom and Y¹ are two adjacent atoms of the ring
with a receptor binding molecule (RBM) having a functional group reactive towards L* of a compound of formula (IIb1), thereby forming a covalent bond between the receptor binding molecule (RBM) and the linker (L) to result in a conjugate of formula (Ib1)

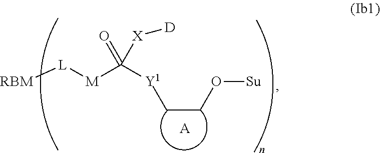

or a pharmaceutically acceptable salt or solvate thereof;
wherein:
RBM, L, M, X, D, Y¹, Su and n are as defined herein;

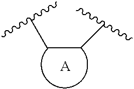

is an optionally substituted four- to seven-membered, preferably five- or six-membered, carbocyclic or heterocyclic ring; and
[symbol] indicate the positions of the oxygen atom and Y¹; preferably the attachment points to the oxygen atom and Y¹ are two adjacent atoms of the ring.

Regarding the methods, in a compound of formula (IIb) or (IIb1) and a conjugate of formula (Ib) or (Ib1) any variable, such as, for example, RBM, L, L*, M, X, D, Y¹,

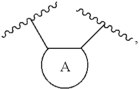

[symbol]

, E, Su and n may be as defined herein.

In some embodiments, the method of preparing a conjugate of formula (I) comprises:
Reacting a compound of formula (IIc)

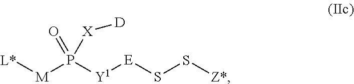

or a pharmaceutically acceptable salt or solvate thereof;
wherein:
L*, M, X, D and Y¹ are as defined herein;
E is a spacer; and
Z* is an optionally substituted aliphatic residue or an optionally substituted aromatic residue;
wherein the sulfur bound to the spacer E, after cleavage of the disulfide bond, is capable of forming a ring, preferably a four- to seven-membered ring, more preferably a five- or six-membered ring, together with the spacer E, Y¹ and the phosphorus;
with a receptor binding molecule (RBM) having a functional group reactive towards L* of a compound of formula (IIc), thereby forming a covalent bond between the receptor binding molecule (RBM) and the linker (L) to result in a conjugate of formula (Ic)

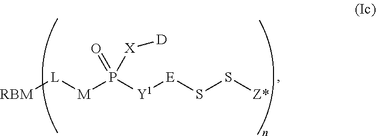

or a pharmaceutically acceptable salt or solvate thereof;
wherein:
RBM, L, M, X, D, Y¹ and n are as defined herein;
E is a spacer; and
Z* is an optionally substituted aliphatic residue or an optionally substituted aromatic residue;
wherein the sulfur bound to the spacer E, after cleavage of the disulfide bond, is capable of forming a ring, preferably a four- to seven-membered ring, more preferably a five- or six-membered ring, together with the spacer E, Y¹ and the phosphorus atom.

In some embodiments, the method of preparing a conjugate of formula (I) comprises:
Reacting a compound of formula (IIc1)

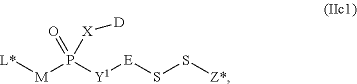

or a pharmaceutically acceptable salt or solvate thereof;
wherein L*, M, X, D, Y¹ and Z* are as defined herein; and
E is

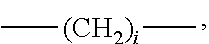

which can be optionally substituted; and wherein i is an integer ranging from 1 to 4; preferably 2, 3 or 4; more preferably 2 or 3; still more preferably 2
with a receptor binding molecule (RBM) having a functional group reactive towards L* of a compound of formula (IIc), thereby forming a covalent bond between the receptor binding molecule (RBM) and the linker (L) to result in a conjugate of formula (Ic)

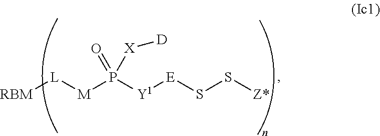

or a pharmaceutically acceptable salt or solvate thereof;
wherein:
RBM, L, M, X, D, Y¹, Z* and n are as defined herein; and
E is

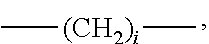

which can be optionally substituted; and wherein i is an integer ranging from 1 to 4; preferably 2, 3 or 4; more preferably 2 or 3; still more preferably 2.

Regarding the methods, in a compound of formula (IIc) or (IIc1) and in a conjugate of formula (Ic) or (Ic1) any variable, such as, for example, L*, L, M, X, D, Y¹, E, i, Z* and n, may be as defined herein.

In some embodiments, the method of preparing a conjugate of formula (I) comprises: reacting a compound of formula (IId)

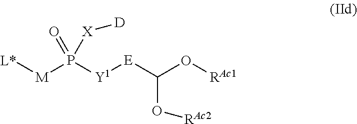

or a pharmaceutically acceptable salt or solvate thereof; wherein L*, M, X, D, Y¹, E, R^{Ac1} and R^{Ac2} are as defined herein, including preferred embodiments thereof, such as, for example, those described above for formula (Id), (Id1) and (IId1);
with a receptor binding molecule (RBM) having a functional group reactive towards L* of a compound of formula (IId), thereby forming a covalent bond between the receptor binding molecule (RBM) and the linker (L) to result in a conjugate of formula (Id)

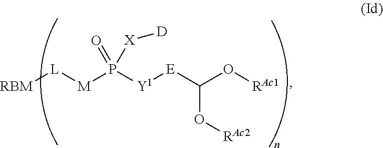

or a pharmaceutically acceptable salt or solvate thereof; wherein
RBM, L, M, X, D, Y¹, E, R^{Ac}1, R^{Ac2} and n are as defined herein, including preferred embodiments thereof, such as, for example, those described above for formula (Id) and (Id1).

In some embodiments, the method of preparing a conjugate of formula (I) comprises: reacting a compound of formula (IIe)

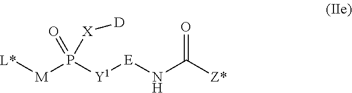

or a pharmaceutically acceptable salt or solvate thereof; wherein L*, M, X, D, Y¹, E and Z* are as defined herein, including preferred embodiments thereof, such as, for example, those described above for formula (Ie); (Ie1) and (IIe1);
with a receptor binding molecule (RBM) having a functional group reactive towards L* of a compound of formula (IIc), thereby forming a covalent bond between the receptor binding molecule (RBM) and the linker (L) to result in a conjugate of formula (Ie)

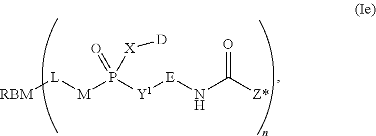

or a pharmaceutically acceptable salt or solvate thereof; wherein:
RBM, L, M, X, D, Y¹, E, Z* and n are as defined herein, including preferred embodiments thereof, such as, for example, those described above for formula (Ie) and (Ie1).

In some embodiments, the method of preparing a conjugate of formula (I) comprises: reacting a compound of formula (IIf)

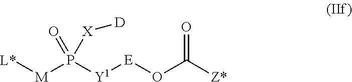

or a pharmaceutically acceptable salt or solvate thereof; wherein L*, M, X, D, Y¹, E and Z* are as defined herein, including preferred embodiments thereof, such as, for example, those described above for formula (If), (If1) and (IIf1);
with a receptor binding molecule (RBM) having a functional group reactive towards L* of a compound of formula (IIc), thereby forming a covalent bond between the receptor binding molecule (RBM) and the linker (L) to result in a conjugate of formula (If)

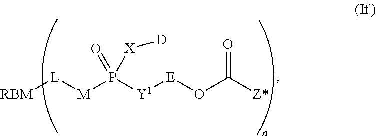

or a pharmaceutically acceptable salt or solvate thereof; wherein:
RBM, L, M, X, D, Y¹, E, Z* and n are as defined herein, including preferred embodiments thereof, such as, for example, those described above for formula (If) and (If1).

In some embodiments, the receptor binding molecule (RBM) may have one or more thiol groups (or also denoted as sulfhydryl groups) which are capable to form a covalent bond via reaction with the linker L* of a compound of formula (II), as described herein. Such receptor binding molecule may be represented by the following formula (III):

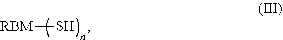

wherein RBM and n are as defined herein. In some preferred embodiments, RBM is an antibody. The one or more thiols groups may be generated, for example, from disulfide bond(s) of the receptor binding molecule by reduction using a suitable reducing agent.

When the receptor binding molecule (RBM) comprises one or more disulfide bridges, such as e.g. an antibody, the method may further comprise reducing at least one disulfide bridge of the receptor binding molecule in the presence of a reducing agent to form a thiol group (SH). The reducing agent may be selected from the group consisting of tris(2-carboxyethyl)phosphine (TCEP), dithiothreitol (DTT), sodium dithionite, sodium thiosulfate, and sodium sulfite. Accordingly, the reducing agent may be dithiothreitol (DTT). The reducing agent may be sodium dithionite. The reducing agent may be sodium sulfite. Preferably, the reducing agent is tris(2-carboxyethyl)phosphine (TCEP).

The reducing of at least one disulfide bridge may comprise using about 1 to about 3 equivalents, preferably about 1 to about 2 equivalents, more preferably about 1 equivalent of the reducing agent per 1 disulfide bridge to be reduced. In this context, it is noted that in theory 1 eq. of the reducing agent, in particular of a reducing agent as described herein, is necessary to reduce 1 disulfide bridge to give 2 thiol groups (SH).

The thiol-containing molecule of formula (III) may be reacted with about 1 to about 4 equivalents, preferably about 1 to about 3 equivalents, more preferably about 1 to about 2 equivalents, still more preferably about 1.5 equivalents of the compound of formula (II) having a linker L* per thiol group (SH). The reaction of a compound of formula (II) having a linker L* with a thiol-containing molecule of formula (III) may be carried out in any suitable reaction medium known to a person skilled in the art, e.g. in an aqueous medium. The reaction of the compound of formula (II) with the thiol-containing molecule of formula (III) may be performed under neutral pH or slightly basic conditions, e.g. the reaction may be performed at a pH of from 6 to 10, or at a pH of from 7 to 9.

Compounds of formula (II) comprising a phosphorus(V) moiety having the following structure, as described herein:

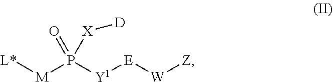

wherein L*, M, X, D, Y¹, E, W and Z are as described herein,
can be prepared, for example, from a phosphorus(II) compound (such as, e.g., tris(diethylamino)phosphine) using substitution reactions at the phosphorus atom to sequentially introduce the moieties L*-M, Y¹-E-W-Z and X-D, and oxidation to obtain a phosphorus(V) compound using an oxidant (such as, e.g., iodine or hydrogen peroxide). Suitable starting materials, reagents and reaction conditions are known and readily selected by a person skilled in the art. A person skilled in the art also knows to use suitable protecting groups, if necessary. A compound of formula (II) is then reacted with the receptor binding molecule, as described herein, in particular by forming a bond between a suitable functional group of the receptor binding molecule and a suitable functional group of L*. As may be the case, if necessary, a protecting group may be removed from a functional group of L* before reacting with the receptor binding molecule, and/or a suitable functional group or a building block comprising a suitable functional group may be attached to the linker L* before reacting with the receptor binding molecule. The Examples section of the present disclosure also comprises guidance on how to prepare or obtain the compounds and conjugates described herein.

### Method of Preparing a Conjugate of Formula (Ia)

The present invention also relates to a method of preparing a conjugate of formula (Ia), said method comprising:
reacting a compound of formula (IIa)

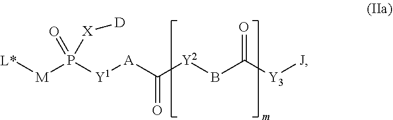

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₃)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

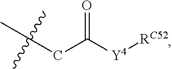

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkyl(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl;
with a receptor binding molecule (RBM) having a functional group reactive towards L* of a compound of formula (IIa), thereby forming a covalent bond between the receptor binding molecule (RBM) and the linker (L) to result in a conjugate of formula (Ia)

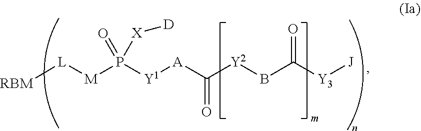

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

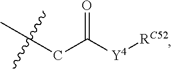

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkyl(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl; and
n is an integer ranging from 1 to 20.

Preferably, the method of preparing a conjugate of formula (Ia) comprises:
Reacting a compound of formula (IIa1)

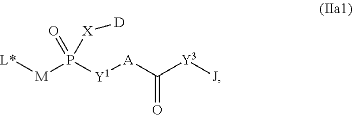

or a pharmaceutically acceptable salt or solvate thereof;
wherein L*, M, X, D, Y¹, A, Y³ and J are as defined herein;
with a receptor binding molecule (RBM) having a functional group reactive towards L* of a compound of formula (IIa1), thereby forming a covalent bond between the receptor binding molecule (RBM) and the linker (L) to result in a conjugate of formula (Ia1)

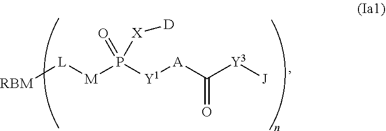

or a pharmaceutically acceptable salt or solvate thereof;
wherein:
RBM, L, M, X, D, Y¹ A, Y³, J and n are as defined herein.

More preferably, the method of preparing a conjugate of formula (Ia) comprises:
Reacting a compound of formula (IIa2):

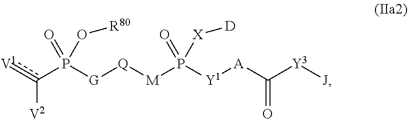

or a pharmaceutically acceptable salt or solvate thereof;
wherein M, X, D, Y¹, A, Y³ and J are as defined herein;
[symbol] is a triple bond; or
[symbol] is a double bond;
V² is absent when [symbol] is a triple bond; or
V² is selected from the group consisting of hydrogen, (C₁-C₈)alkyl, (C₆-C₁₀)aryl, and (C₁-C₈)alkylene(C₆-C₁₀)aryl when [symbol] is a double bond;
V¹ is R^{V11—C} when [symbol] is a triple bond; or
V¹ is

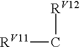

when [symbol] is a double bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl; and
R⁸⁰ is an optionally substituted aliphatic residue or an optionally substituted aromatic residue
with a thiol-containing molecule of formula (III)

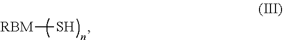

wherein RBM is a receptor binding molecule; and
n is an integer ranging from 1 to 20;
to result in a conjugate of formula (Ia2)

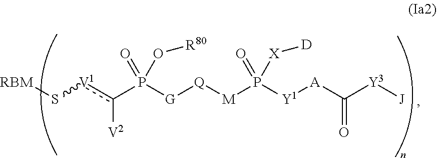

or a pharmaceutically acceptable salt or solvate thereof;
wherein:
RBM, M, X, D, Y¹, A, Y³, J and n are as defined herein;
[symbol] is a double bond when [symbol] in a compound of formula (IIa2) is a triple bond; or
[symbol] is a bond when [symbol] a compound of formula (IIa2) is a double bond;
V² is absent when [symbol] is a double bond; or
V² is selected from the group consisting of hydrogen, (C₁-C₈)alkyl, (C₆-C₁₀)aryl, and (C₁-C₈)alkylene(C₆-C₁₀)aryl when [symbol] is a bond;
V¹ is R^{V11—C} when [symbol] is a double bond; or
V¹ is

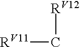

when [symbol] is a bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl; and
R⁸⁰ is an optionally substituted aliphatic residue or an optionally substituted aromatic residue.

Regarding the methods, in a compound of formula (IIa), (IIa1) or (IIa2) and in a conjugate of formula (Ia), (Ia1) or (Ia2) any variable, such as, for example, RBM, L, L*, V¹, V², [symbol] , [symbol] , R⁸⁰, G, Q, M, X, D, Y¹, A, Y², B, Y³, J, m and n may be as defined herein.

### Method of Preparing a Conjugate of Formula (Ib1)

The present invention also relates to a method of preparing a conjugate of formula (Ib1), said method comprising:
Reacting a compound of formula (IIb1):

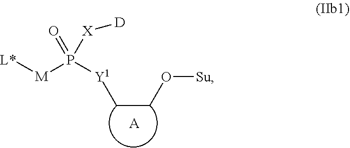

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;

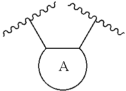

is an optionally substituted four- to seven-membered, preferably five- or six-membered, carbocyclic or heterocyclic ring; and
[symbol] indicate the positions of the oxygen atom and Y¹; preferably the attachment points to the oxygen atom and Y¹ are two adjacent atoms of the ring; and
Su is a sugar moiety
with a receptor binding molecule (RBM) having a functional group reactive towards L* of a compound of formula (IIb1), thereby forming a covalent bond between the receptor binding molecule (RBM) and the linker (L) to result in a conjugate of formula (Ib1)

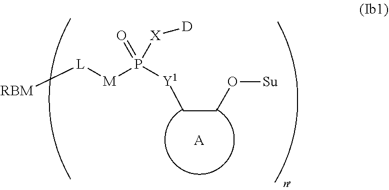

or a pharmaceutically acceptable salt or solvate thereof;
wherein:
RBM is a receptor binding molecule;
L is a linker;
M is O, NR^{M60}, or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;

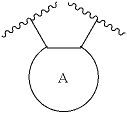

is an optionally substituted four- to seven-membered, preferably five- or six-membered, carbocyclic or heterocyclic ring;
[symbol] indicate the positions of the oxygen atom and Y¹; preferably the attachment points to the oxygen atom and Y¹ are two adjacent atoms of the ring;
Su is a sugar moiety; and
n is an integer ranging from 1 to 20.

Regarding the methods, in a compound of formula (IIb1) and a conjugate of formula (Ib1) any variable, such as, for example, RBM, L, L*, M, X, D, Y¹,

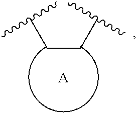

[symbol]

, Su and n may be as defined herein.

### Method of Preparing a Conjugate of Formula (Ic1)

The present invention also relates to a method of preparing a conjugate of formula (Ic1), comprising:
Reacting a compound of formula (IIc1)

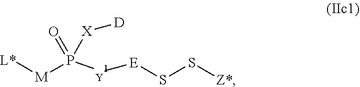

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

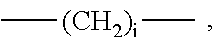

which can be optionally substituted; and wherein i is an integer ranging from 1 to 4; preferably 2, 3 or 4; more preferably 2 or 3; still more preferably 2; and
Z* is an optionally substituted aliphatic residue or an optionally substituted aromatic residue.
with a receptor binding molecule (RBM) having a functional group reactive towards L* of a compound of formula (IIc), thereby forming a covalent bond between the receptor binding molecule (RBM) and the linker (L) to result in a conjugate of formula (Ic)

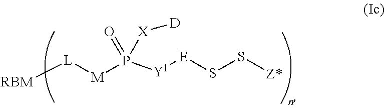

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

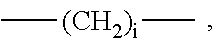

which can be optionally substituted; and wherein i is an integer ranging from 1 to 4; preferably 2, 3 or 4; more preferably 2 or 3; still more preferably 2;
Z* is an optionally substituted aliphatic residue or an optionally substituted aromatic residue; and
n is an integer ranging from 1 to 20.

Regarding the methods, in a compound of formula (IIc1) and a conjugate of formula (Ic1) any variable, such as, for example, RBM, L, L*, M, X, D, Y¹, E, i, Z* and n, may be as defined herein.

The present invention also relates to a conjugate of the invention obtainable or being obtained by any method of preparing a conjugate of the invention as described herein.

### Pharmaceutical Composition

The present invention further relates to a pharmaceutical composition comprising a conjugate of the invention.

The pharmaceutical composition may comprise a population of a conjugate of the invention, wherein the average number of drug moieties (D) per receptor binding molecule (RBM) in the composition is from more than 0 to about 14, preferably from about 1 to about 14, more preferably from about 2 to about 14, still more preferably from about 4 to about 14, still more preferably from about 5 to about 12, still more preferably from about 6 to about 12, still more preferably from about 6 to about 10, even more preferably about 8. Accordingly, the pharmaceutical composition may comprise a population of a conjugate of the invention, wherein the average number of drug moieties (D) per receptor binding molecule (RBM) in the composition is from more than 0 to about 14. Preferably, the pharmaceutical composition comprises a population of a conjugate of the invention, wherein the average number of drug moieties (D) per receptor binding molecule (RBM) in the composition is from about 1 to about 14. More preferably, the pharmaceutical composition comprises a population of a conjugate of the invention, wherein the average number of drug moieties (D) per receptor binding molecule (RBM) in the composition is from about 2 to about 14. Still more preferably, the pharmaceutical composition comprises a population of a conjugate of the invention, wherein the average number of drug moieties (D) per receptor binding molecule (RBM) in the composition is from about 4 to about 14. Still more preferably, the pharmaceutical composition comprises a population of a conjugate of the invention, wherein the average number of drug moieties (D) per receptor binding molecule (RBM) in the composition is from about 5 to about 12. Still more preferably, the pharmaceutical composition comprises a population of a conjugate of the invention, wherein the average number of drug moieties (D) per receptor binding molecule (RBM) in the composition is from about 6 to about 12. Still more preferably, the pharmaceutical composition comprises a population of a conjugate of the invention, wherein the average number of drug moieties (D) per receptor binding molecule (RBM) in the composition is from about 6 to about 10. Even more preferably, the pharmaceutical composition comprises a population of a conjugate of the invention, wherein the average number of drug moieties (D) per receptor binding molecule (RBM) in the composition is about 8. When the receptor binding molecule (RBM) is, in some preferred embodiments, an antibody or an antibody fragment, such average number is also denoted as “average drug to antibody ratio (DARav)”. In this context, a person skilled in the art understands that a composition may comprise a population of conjugates, which may differ in the number of drug moieties (D) per receptor binding molecule (RBM), and which may optionally also comprise unconjugated receptor binding molecule, so that the result is an average number of drug moieties per receptor binding molecule.

The pharmaceutical composition may comprise a population of a conjugate of the invention, wherein the average number of drug moieties (D) per receptor binding molecule (RBM) is from more than 0 to about 14, preferably from about 1 to about 14, more preferably from about 1 to about 12, still more preferably, from about 2 to about 10, still more preferably from about 2 to about 8, still more preferably from about 2 to about 6, still more preferably from about 3 to about 5, even more preferably about 4. Accordingly, the pharmaceutical composition may comprise a population of a conjugate of the invention, wherein the average number of drug moieties (D) per receptor binding molecule (RBM) is from more than 0 to about 14. Preferably, the pharmaceutical composition comprises a population of a conjugate of the invention, wherein the average number of drug moieties (D) per receptor binding molecule (RBM) is from about 1 to about 14. More preferably, the pharmaceutical composition comprises a population of a conjugate of the invention, wherein the average number of drug moieties (D) per receptor binding molecule (RBM) is from about 1 to about 12. Still more preferably, the pharmaceutical composition comprises a population of a conjugate of the invention, wherein the average number of drug moieties (D) per receptor binding molecule (RBM) is from about 2 to about 10. Still more preferably, the pharmaceutical composition comprises a population of a conjugate of the invention, wherein the average number of drug moieties (D) per receptor binding molecule (RBM) is from about 2 to about 8. Still more preferably, the pharmaceutical composition comprises a population of a conjugate of the invention, wherein the average number of drug moieties (D) per receptor binding molecule (RBM) is from about 2 to about 6. Still more preferably, the pharmaceutical composition comprises a population of a conjugate of the invention, wherein the average number of drug moieties (D) per receptor binding molecule (RBM) is from about 3 to about 5. Even more preferably, the pharmaceutical composition comprises a population of a conjugate of the invention, wherein the average number of drug moieties (D) per receptor binding molecule (RBM) is about 4. When the receptor binding molecule (RBM) is, in some preferred embodiments, an antibody or an antibody fragment, such average number is also denoted as “average drug to antibody ratio (DARav)”.

The pharmaceutical composition may further comprise one or more pharmaceutically acceptable carriers. In a specific embodiment, the term “pharmaceutically acceptable” means approved by a regulatory agency or other generally recognized pharmacopoeia for use in animals, and more particularly in humans. Pharmaceutically acceptable carriers are well known in the art and include, for example, aqueous solutions such as water, 5% dextrose, or physiologically buffered saline or other solvents or vehicles such as glycols, glycerol, oils such as olive oil, or injectable organic esters that are suitable for administration to a human or non-human subject. Particular exemplary pharmaceutically acceptable carriers include (biodegradable) liposomes; microspheres made of the biodegradable polymer poly(D,L-lactic-coglycolic acid (PLGA), albumin microspheres; synthetic polymers (soluble); nanofibers, protein-DNA complexes; protein conjugates; erythrocytes; or virosomes. Various carrier based dosage forms comprise solid lipid nanoparticles (SLNs), polymeric nanoparticles, ceramic nanoparticles, hydrogel nanoparticles, copolymerized peptide nanoparticles, nanocrystals and nanosuspensions, nanocrystals, nanotubes and nanowires, functionalized nanocarriers, nanospheres, nanocapsules, liposomes, lipid emulsions, lipid microtubules/microcylinders, lipid microbubbles, lipospheres, lipopolyplexes, inverse lipid micelles, dendrimers, ethosomes, multicomposite ultrathin capsules, aquasomes, pharmacosomes, colloidosomes, niosomes, discomes, proniosomes, microspheres, microemulsions and polymeric micelles. Other suitable pharmaceutically acceptable carriers and excipients are inter alia described in Remington's Pharmaceutical Sciences, 15^{th} Ed., Mack Publishing Co., New Jersey (1991) and Bauer et al., Pharmazeutische Technologie, 5^{th} Ed., Govi-Verlag Frankfurt (1997). see, e.g., Remington: The Science and Practice of Pharmacy, 21^{st} edition; Lippincott Williams & Wilkins, 2005.

In some embodiments, a pharmaceutically acceptable carrier or composition is sterile. A pharmaceutical composition can comprise, in addition to the active agent, physiologically acceptable compounds that act, for example, as bulking agents, fillers, solubilizers, stabilizers, osmotic agents, uptake enhancers, etc. Physiologically acceptable compounds include, for example, carbohydrates, such as glucose, sucrose, lactose; dextrans; polyols such as mannitol; antioxidants, such as ascorbic acid or glutathione; preservatives; chelating agents; buffers; or other stabilizers or excipients.

The choice of a pharmaceutically acceptable carrier(s) and/or physiologically acceptable compound(s) can depend for example, on the nature of the active agent, e.g., solubility, compatibility (meaning that the substances can be present together in the composition without interacting in a manner that would substantially reduce the pharmaceutical efficacy of the pharmaceutical composition under ordinary use situations) and/or route of administration of the composition.

Pharmaceutical compositions of the invention may comprise a therapeutically effective amount of the conjugate of the invention described herein and can be structured in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for topical or oral administration. A variety of routes are applicable for administration of the conjugate of the invention, including, but not limited to, orally, topically, transdermally, subcutaneously, intravenously, intraperitoneally, intramuscularly or intraocularly. However, any other route may readily be chosen by the person skilled in the art if desired.

### Use in Methods of Treatment

Conjugates of the present invention can be used for the treatment, in particular the treatment of cancer. Accordingly, the present invention further relates to a conjugate of the invention for use in a method of treating a disease, optionally comprising the administration of an effective amount of the conjugate of the invention to a subject or patient in need thereof. Also, the present invention relates to a pharmaceutical composition of the invention for use in a method of treating a disease, optionally comprising the administration of an effective amount of the pharmaceutical composition of the invention to a subject or patient in need thereof. The disease may be associated with overexpression of Trop2. The disease may be associated with overexpression of Her2. The disease may be cancer. The cancer may be a solid tumor. The disease may be a cancer associated with overexpression of Trop2. The disease may be a cancer associated with overexpression of Her2.

The present invention also relates to the use of a conjugate of the invention for the manufacture of a medicament for treating a disease. The present invention also relates to the use of a pharmaceutical composition of the invention for the manufacture of a medicament for treating a disease. The disease may be associated with overexpression of Trop2. The disease may be associated with overexpression of Her2. The disease may be cancer. The cancer may be a solid tumor. The disease may be a cancer associated with overexpression of Trop2. The disease may be a cancer associated with overexpression of Her2.

The present invention also relates to a method of treating a disease, comprising the administration of an effective amount of a conjugate of the invention to a subject or patient in need thereof. The present invention also relates to a method of treating a disease, comprising the administration of an effective amount of a pharmaceutical composition of the invention to a subject or patient in need thereof. The disease may be associated with overexpression of Trop2. The disease may be associated with overexpression of Trop2. The disease may be cancer. The cancer may be a solid tumor. The disease may be a cancer associated with overexpression of Trop2. The disease may be a cancer associated with overexpression of Her2.

The phrase “effective amount” in general refers to an amount of a therapeutic agent (e.g., the conjugate of the invention) that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays. The exact amount will depend on the purpose of the treatment, and will be ascertainable by one skilled in the art using known techniques (see, for example, Lloyd (1999) The Art, Science and Technology of Pharmaceutical Compounding).

Further, the present invention relates to a conjugate as described herein for use in a method of treating cancer in a patient. The present invention also relates to a pharmaceutical composition as described herein for use in a method of treating cancer in a patient. The term “patient” means according to the invention a human being, a non-human primate or another animal, in particular a mammal such as a cow, horse, pig, sheep, goat, dog, cat or a rodent such as a mouse and rat. In a particularly preferred embodiment, the patient is a human being. Except when noted, the terms “patient” or “subject” are used herein interchangeably. The term “treatment” in all its grammatical forms includes therapeutic or prophylactic treatment. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of diseases.

A conjugate of the present invention may be administered at any dose that is therapeutically effective. The upper limit is usually a dose that is still safe to administer in terms of side effects. As illustrative examples a conjugate of the invention may be administered at a(n effective) dose of 50 mg/kg, 45 mg/kg, 40 mg/kg, 35 mg/kg, 30 mg/kg, 25 mg/kg, 20 mg/kg, 18 mg/kg, 16 mg/kg, 14 mg/kg, 12 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg or 0.25 mg/kg. This dose may be administered over a given amount of time, for example over two to four weeks (14 to 28 days), a period that is also called “treatment cycle”. Such a treatment cycle may be repeated, depending on the disease progression or regression, i.e. treatment outcome. In line with this, a conjugate of the invention may be administered to a patient by any suitable way, for example by injection or infusion such as intravenous infusion. In particular, a conjugate of the invention may be administered at a(n effective) dose of 5 mg/kg given as an intravenous infusion, e.g., once every three weeks.

The term “cancer”, as used herein, can denote any cancer, e.g., preferably said cancer is selected from the group consisting of: Breast cancer, Head and neck cancer, Ovary cancer, Endometrium cancer, Uterine cervix cancer, Rectum cancer, Colon cancer, Esophagus cancer, Stomach cancer, Lung cancer, Kidney cancer, Adrenal gland cancer, Bladder cancer, Liver cancer, Sarcoma, Brain cancer, Nevi and Melanomas, Urogenital cancer, Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers, Retinoblastom, Thyroid cancer, Fallopian tube cancer; further preferably said cancer is a solid cancer, e.g., selected from the group consisting of: Breast cancer, Head and neck cancer, Ovarian cancer, Endometrial cancer, Uterus cancer (e.g., including cancers of the muscle sheets), Cervical cancer, Rectum cancer, Colon cancer, Anal cancer, Esophagus cancer, Stomach cancer, Lung cancer, Kidney cancer, Adrenal gland cancer, Bladder cancer, Liver cancer, Sarcoma (e.g., including osteosarcoma and Kaposi sarcoma), Brain cancer (e.g., including pituitary tumor/s), Nevi and Melanoma cancers, Skin cancers (e.g., including squamous cell carcinoma and melanoma), Urogenital cancer (e.g., ureter and bladder cancer, testicular cancer, prostate cancer, penile cancer), Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers (e.g., including lymphoma, leukemia, myeloma, Myelodysplastic syndromes, myelofibrosis), Eye cancers (e.g., including Retinoblastoma), Neuroendocrine tumors, Cancer of unknown primary (CUP)). Preferably said cancer is a solid and/or metastatic cancer, further preferably said cancer is selected from the group consisting of: lung cancer, ovarian cancer, thyroid cancer, nonsquamous non-small cell lung carcinoma, nonmucinous ovarian carcinoma, papillary thyroid carcinoma, renal cancer, endometrial cancer, uterus cancer, ureter cancer, bladder cancer and fallopian tube cancer. Said cancer may be also selected from the group consisting of Cancer in Adolescents, Adrenocortical Carcinoma, Anal Cancer, Astrocytomas, Atypical Teratoid/Rhabdoid Tumor, Basal Cell Carcinoma, Bile Duct Cancer, Bladder Cancer, Bone Cancer, Brain Tumors, Breast Cancer, Bronchial Tumors, Cervical Cancer, Chordoma, Chronic Myeloproliferative Neoplasms, Colorectal Cancer, Craniopharyngioma, Embryonal Tumors, Medulloblastoma and Other Central Nervous System, Childhood (Brain Cancer), Endometrial Cancer (Uterine Cancer), Ependymoma, Childhood (Brain Cancer), Esophageal Cancer, Esthesioneuroblastoma (Head and Neck Cancer), Ewing Sarcoma (Bone Cancer), Extracranial Germ Cell Tumor, Childhood, Extragonadal Germ Cell Tumor, Fallopian Tube Cancer, Gallbladder Cancer, Gastric (Stomach) Cancer, Gastrointestinal Carcinoid Tumor, Gastrointestinal Stromal Tumors (GIST), Gestational Trophoblastic Disease, Head and Neck Cancer, Heart Tumors, Hepatocellular (Liver) Cancer, Histiocytosis, Langerhans Cell, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Lip and Oral Cavity Cancer (Head and Neck Cancer), Liver Cancer, Lung Cancer, Male Breast Cancer, Melanoma, Melanoma, Intraocular (Eye), Merkel Cell Carcinoma (Skin Cancer), Mesothelioma, Malignant, Mouth Cancer (Head and Neck Cancer), Multiple Endocrine Neoplasia Syndromes, Nasal Cavity and Paranasal Sinus Cancer (Head and Neck Cancer), Nasopharyngeal Cancer (Head and Neck Cancer), Neuroblastoma, Non-Small Cell Lung Cancer, Oral Cancer, Lip and Oral Cavity Cancer and Oropharyngeal Cancer (Head and Neck Cancer), Osteosarcoma, Ovarian Cancer, Pancreatic Cancer, Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Pleuropulmonary Blastoma (Lung Cancer), Primary Central Nervous System (CNS) Lymphoma, Primary Peritoneal Cancer, Prostate Cancer, Pulmonary Inflammatory Myofibroblastic Tumor (Lung Cancer), Rectal Cancer, Renal Cell (Kidney) Cancer, Retinoblastoma, Rhabdomyosarcoma, Childhood (Soft Tissue Sarcoma), Salivary Gland Cancer (Head and Neck Cancer), Skin Cancer, Small Cell Lung Cancer, Small Intestine Cancer, Soft Tissue Sarcoma, Squamous Cell Carcinoma of the Skin —see Skin Cancer, Squamous Neck Cancer with Occult Primary, Metastatic (Head and Neck Cancer), Stomach (Gastric) Cancer, Testicular Cancer, Thymoma and Thymic Carcinoma, Thyroid Cancer, Tracheobronchial Tumors (Lung Cancer), Transitional Cell Cancer of the Renal Pelvis and Ureter (Kidney (Renal Cell) Cancer), Ureter and Renal Pelvis, Transitional Cell Cancer (Kidney (Renal Cell) Cancer, Urethral Cancer, Uterine Cancer, Endometrial, Uterine Sarcoma, Vaginal Cancer, Vascular Tumors (Soft Tissue Sarcoma), Vulvar Cancer, and Wilms Tumor and Other Childhood Kidney Tumors.

By “tumor” is meant a group of cells or tissue that is formed by misregulated cellular proliferation, in particular cancer. Tumors may show partial or complete lack of structural organization and functional coordination with the normal tissue, and usually form a distinct mass of tissue, which may be either benign or malignant. In particular, the term “tumor” refers to a malignant tumor. The term “tumor” may refer to a solid tumor. According to one embodiment, the term “tumor” or “tumor cell” also refers to non-solid cancers and cells of non-solid cancers such as leukemia cells. According to another embodiment, respective non-solid cancers or cells thereof are not encompassed by the terms “tumor” and “tumor cell”.

By “metastasis” is meant the spread of cancer cells from its original site to another part of the body. The formation of metastasis is a very complex process and normally involves detachment of cancer cells from a primary tumor, entering the body circulation and settling down to grow within normal tissues elsewhere in the body. When tumor cells metastasize, the new tumor is called a secondary or metastatic tumor, and its cells normally resemble those in the original tumor. This means, for example, that, if breast cancer metastasizes to the lungs, the secondary tumor is made up of abnormal breast cells, not of abnormal lung cells. The tumor in the lung is then called metastatic breast cancer, not lung cancer.

### Items of the Invention

The invention further relates to the following items:

1. A conjugate having the formula (I):

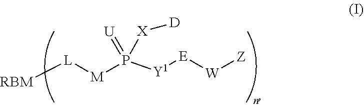

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group;
W is a moiety which, after cleavage of the group Z, is capable of forming a ring together with the spacer E, Y¹ and the phosphorus; and
n is an integer ranging from 1 to 20.

1a. The conjugate of item 1, wherein W is a moiety which, after cleavage of the group Z, is capable of forming a four- to seven-membered ring together with the spacer E, Y¹ and the phosphorus.

1b. The conjugate of item 1a, wherein W is a moiety which, after cleavage of the group Z, is capable of forming a five- or six-membered ring together with the spacer E, Y¹ and the phosphorus.

2. The conjugate of any one of items 1 to 1b, having the formula (Ia):

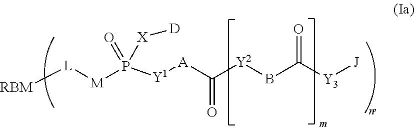

or a pharmaceutically acceptable salt or solvate thereof;
wherein:
RBM, L, M, X, D, Y¹ and n are as defined in item 1;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

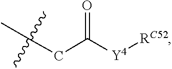

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl.

3. The conjugate of item 2, wherein m is an integer ranging from 0 to 12, preferably from 0 to 10, more preferably from 0 to 8, still more preferably from 0 to 5, even more preferably from 0 to 3.

4. The conjugate of item 2 or item 3, wherein m is 0.

5. The conjugate of item 4, wherein the conjugate has formula (Ia1):

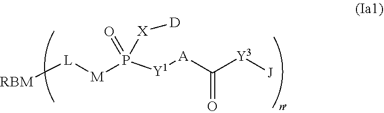

or a pharmaceutically acceptable salt or solvate thereof;
wherein RBM, L, M, X, D, Y¹, A, Y³, J and n are as defined in any one of the preceding items.

6. The conjugate of any one of items 2 to 5, wherein Y¹ is NR^{A20} or O, wherein R^{A20} is as defined in any one of the preceding items;
preferably wherein Y¹ is NH or O;
more preferably wherein Y¹ is NH.

7. The conjugate of any one of items 2 to 6, wherein A is CR^{A30}R^{A31}, wherein R^{A30} and R^{A31} are as defined in any one of the preceding items.

8. The conjugate of any one of items 2 to 7, wherein R^{A30} and R^{A31} are each independently selected from the group consisting of hydrogen, (C₁-C₈)alkyl, (C₆-C₁₀)aryl, and (C₁-C₈)alkylene(C₆-C₁₀)aryl;
preferably wherein R^{A30} and R^{A31} are each independently selected from the group consisting of hydrogen, (C₁-C₈)alkyl, and (C₁-C₈)alkylene(C₆-C₁₀)aryl;
more preferably wherein R^{A30} and R^{A31} are each independently selected from the group consisting of hydrogen and (C₁-C₈)alkyl;
more preferably wherein R^{A30} and R^{A31} are each independently selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl;
still more preferably wherein R^{A30} and R^{A31} are each independently selected from hydrogen and CH₃.

9. The conjugate of any one of items 2 to 8, wherein R^{A30} is hydrogen and R^{A31} is selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; preferably wherein R^{A30} is hydrogen and R^{A31} is selected from the group consisting of hydrogen, (C₁-C₈)alkyl, (C₆-C₁₀)aryl, and (C₁-C₈)alkylene(C₆-C₁₀)aryl;
more preferably wherein R^{A30} is hydrogen and R^{A31} is selected from the group consisting of (C₁-C₈)alkyl, and (C₁-C₈)alkylene(C₆-C₁₀)aryl;
more preferably wherein R^{A30} is hydrogen and R^{A31} is (C₁-C₈)alkyl;
still more preferably wherein R^{A30} is hydrogen and R^{A31} is selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl;
even more preferably wherein R^{A30} is hydrogen and R^{A31} is CH₃.

10. The conjugate of any one of items 7 to 9, wherein Y¹ is NR^{A20} or O, wherein R^{A20} is as defined in any one of the preceding items;
preferably wherein Y¹ is NH or O;
more preferably wherein Y¹ is NH.

11. The conjugate of any one of items 2 to 10, wherein Y³ is O.

12. The conjugate of any one of items 2 to 10, wherein Y³ is NR^{C40}, wherein R^{C40} is as defined in any one of the preceding items;
preferably wherein Y³ is NH.

13. The conjugate of any one of items 2 to 12, wherein J is

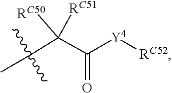

wherein R^{C50}, R^{C51}, Y⁴, R^{C52} and [symbol] are as defined in any one of the preceding items.

14. The conjugate of any one of items 2 to 13, wherein Y⁴ is O or NR^{C53}, wherein R^{C53} is as defined in any one of the preceding items;
preferably wherein Y⁴ is O or NH;
more preferably wherein Y⁴ is O.

15. The conjugate of any one of items 2 to 14, wherein R^{C50} and R^{C51} are each independently selected from the group consisting of hydrogen, (C₁-C₈)alkyl, (C₆-C₁₀)aryl, and (C₁-C₈)alkylene(C₆-C₁₀)aryl;
preferably wherein R^{C50} and R^{C51} are each independently selected from the group consisting of hydrogen, (C₁-C₈)alkyl, and (C₁-C₈)alkylene(C₆-C₁₀)aryl;
more preferably wherein R^{C50} and R^{C51} are each independently selected from the group consisting of hydrogen and (C₁-C₈)alkyl;
still more preferably wherein R^{C50} and R^{C51} are each independently selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl;
even more preferably wherein R^{C50} and R^{C51} are each independently selected from hydrogen and CH₃.

16. The conjugate of any one of items 2 to 15, wherein R^{C50} is hydrogen and R^{C51} is selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; preferably wherein R^{C50} is hydrogen and R^{C51} is selected from the group consisting of hydrogen, (C₁-C₈)alkyl, (C₆-C₁₀)aryl, and (C₁-C₈)alkylene(C₆-C₁₀)aryl;
more preferably wherein R^{C50} is hydrogen and R^{C51} is selected from the group consisting of (C₁-C₈)alkyl, and (C₁-C₈)alkylene(C₆-C₁₀)aryl;
still more wherein R^{C50} is hydrogen and R^{C51} is (C₁-C₈)alkyl;
still more preferably wherein R^{C50} is hydrogen and R^{C51} is selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl;
even more preferably wherein R^{C50} is hydrogen and R^{C51} is CH₃.

17. The conjugate of any one of items 2 to 16, wherein R^{C52} is selected from the group consisting of hydrogen, (C₁-C₈)alkyl, (C₆-C₁₀)aryl, and (C₁-C₈)alkylene(C₆-C₁₀)aryl; preferably wherein R^{C52} is selected from the group consisting of hydrogen, (C₁-C₈)alkyl, and (C₁-C₈)alkylene(C₆-C₁₀)aryl;
more preferably wherein R^{C52} is selected from the group consisting of hydrogen and (C₁-C₈)alkyl;
still more preferably wherein R^{C52} is selected from the group consisting of hydrogen, CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl;
still more preferably wherein R^{C52} is selected from the group consisting of hydrogen, CH(CH₃)₂ and C(CH₃)₃;
even more preferably wherein R^{C52} is hydrogen.

18. The conjugate of any one of items 13 to 17, wherein Y³ is NR^{C40}, wherein R^{C40} is as defined in any one of the preceding items;
preferably wherein Y³ is NH or O;
more preferably wherein Y³ is NH.

19. The conjugate of any one of items 2 to 18, wherein A is CR^{A30}R^{A31} and J is

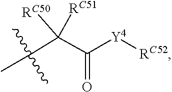

wherein R^{A30}, R^{A31}, R^{C50}, R^{C51}, Y⁴, R^{C52} and [symbol] are as defined in any one of the preceding items;
preferably wherein m is 0.

20. The conjugate of item 19, wherein Y¹ is NR^{A20}, Y³ is NR^{C40}, and Y⁴ is O, wherein R^{A20} and R^{C40} are as defined in any one of the preceding items;
preferably wherein Y¹ is NH, Y³ is NH and Y⁴ is O;
preferably wherein m is 0.

21. The conjugate of item 20, wherein R^{A30} is hydrogen, R^{A31} is CH₃, R^{C50} is hydrogen, R^{C51} is CH₃ and R^{C52} is hydrogen.

22. The conjugate of any one of items 2 to 12, wherein J is selected from the group consisting of (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl;
preferably wherein J is selected from the group consisting of (C₁-C₈)alkyl, (C₆-C₁₀)aryl, and (C₁-C₈)alkylene(C₆-C₁₀)aryl;
more preferably wherein J is selected from the group consisting of (C₁-C₈)alkyl and (C₁-C₈)alkylene(C₆-C₁₀)aryl;
still more preferably wherein J is (C₁-C₈)alkyl;
still more preferably wherein J is selected from the group consisting of CH₃, CH₂CH₃, CH₂CH₃CH₃, CH(CH₃)₂, CH₂CH₂CH₂CH₃, CH(CH₃)CH₂CH₃, CH₂CH(CH₃)₂, C(CH₃)₃, and benzyl;
even more preferably wherein J is CH(CH₃)₂ or C(CH₃)₃;
preferably wherein m is 0.

23. The conjugate of item 22, wherein Y³ is O or NR^{C40}, wherein R^{C40} is as defined in any one of the preceding items;
preferably wherein Y³ is O or NH;
more preferably wherein Y³ is O;
preferably wherein m is 0.

24. The conjugate of item 22 or item 23, wherein A is CR^{A30}R^{A31} wherein R^{A30} and R^{A31} are as defined in any one of the preceding items.

25. The conjugate of item 24, wherein Y¹ is NR^{A20} and Y³ is O, wherein R^{A20} is as defined in any one of the preceding items;
preferably wherein Y¹ is NH and Y³ is O;
preferably wherein m is 0.

26. The conjugate of item 25, wherein R^{A30} is H, R^{A31} is CH₃, Y¹ is NH, and Y³ is O;
preferably wherein J is CH(CH₃)₂ or C(CH₃)₃;
preferably wherein m is 0.

27. The conjugate of any one of items 1 to 1b having the formula (Ib):

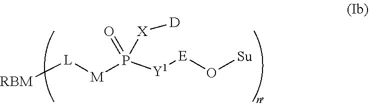

or a pharmaceutically acceptable salt or solvate thereof;
wherein:
RBM, L, M, X, D, Y¹ and n are as defined in item 1;
E is a spacer;
Su is a sugar moiety which is bound to the oxygen atom via a cleavable bond; and
wherein the oxygen, after cleavage of the sugar moiety Su, is capable of forming a ring together with the spacer E, Y¹ and the phosphorus.

27a. The conjugate of item 27, wherein the sugar moiety Su is protected or unprotected.

28. The conjugate of item 27 or 27a, wherein the sugar moiety Su is glucuronic acid:

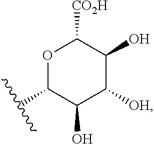 [SMILES: *[C@@H]1O[C@H](C(=O)O)[C@@H](O)[C@H](O)[C@H]1O]

wherein [symbol] indicates the position of the oxygen atom.

29. The conjugate of item 27, 27a or 28, wherein the spacer E has the following structure A:

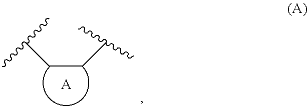

which is an optionally substituted four- to seven-membered, preferably five- or six-membered, carbocyclic or heterocyclic ring;
wherein [symbol] indicate the positions of the oxygen atom and Y¹; and
wherein preferably the attachment points of A to the oxygen atom and Y¹ are two adjacent atoms of the ring.

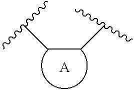

30. The conjugate of item 29, wherein has the following structure:

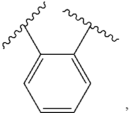 [SMILES: *c1ccccc1*]

wherein [symbol] indicate the positions of the oxygen atom and Y¹.

31. The conjugate of any one of items 27 to 30, wherein Y¹ is NR^{A20} or O, wherein R^{A20} is as defined in any one of the preceding items;
preferably wherein Y¹ is NH or O;
more preferably wherein Y¹ is NH.

32. The conjugate of item 1 having the formula (Id):

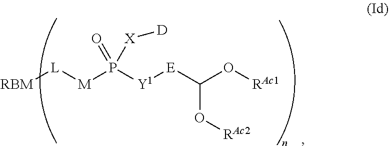

or a pharmaceutically acceptable salt or solvate thereof;
wherein:
RBM, L, M, X, D, Y¹ and n are as defined in claim 1;
E is a spacer; and
R^{Ac1} and R^{Ac2} are each independently an optionally substituted aliphatic residue or an optionally substituted aromatic residue; optionally, R^{Ac1} and R^{Ac2} can together with the oxygen atoms and the carbon atom form a 3- to 8-membered ring.

32a. The conjugate of item 32, wherein R^{Ac1} and R^{Ac2} are each independently optionally substituted (C₁-C₈)alkyl, preferably R^{Ac1} and R^{Ac2} are each independently methyl, ethyl, propyl such as, e.g., iso-propyl or butyl such as, e.g., tert-butyl.

32b. The conjugate of item 32 or 32a, wherein R^{Ac1} and R^{Ac2} are the same.

32c. The conjugate of any one of items 32 to 32b, wherein Y¹ is NR^{A20} or O, wherein R^{A20} is as defined in any one of the preceding items;
preferably wherein Y¹ is NH or O;
more preferably wherein Y¹ is NH.

32d. The conjugate of any one of items 1 to 1b having the formula (Ic):

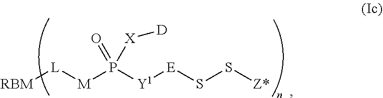

or a pharmaceutically acceptable salt or solvate thereof;
wherein:
RBM, L, M, X, D, Y¹ and n are as defined in item 1;
E is a spacer;
Z* is an optionally substituted aliphatic residue or an optionally substituted aromatic residue; and
wherein the sulfur bound to the spacer E, after cleavage of the disulfide bond, is capable of forming a ring together with the spacer E, Y¹ and the phosphorus.

32e. The conjugate of item 1 having the formula (Ie):

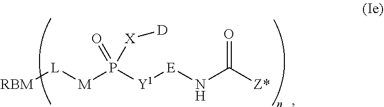

or a pharmaceutically acceptable salt or solvate thereof;
wherein:
RBM, L, M, X, D, Y¹ and n are as defined in item 1;
E is a spacer;
Z* is an optionally substituted aliphatic residue or an optionally substituted aromatic residue; and
wherein the nitrogen atom bound to the spacer E, after cleavage of the acetyl bond, is capable of forming a ring, preferably a four- to seven-membered ring, more preferably a five-or six-membered ring, together with the spacer E, Y¹ and the phosphorus.

32f. The conjugate of item 1 having the formula (If):

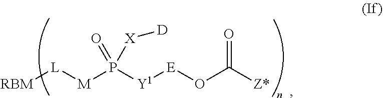

or a pharmaceutically acceptable salt or solvate thereof;
wherein:
RBM, L, M, X, D, Y¹ and n are as defined in item 1;
E is a spacer;
Z* is an optionally substituted aliphatic residue or an optionally substituted aromatic residue; and
wherein the oxygen atom bound to the spacer E, after cleavage of the acetyl bond, is capable of forming a ring, preferably a four- to seven-membered ring, more preferably a five-or six-membered ring, together with the spacer E, Y¹ and the phosphorus.

33. The conjugate of any one of items 32d to 32f, wherein the spacer E is

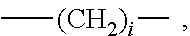

which can be optionally substituted; and
wherein i is an integer ranging from 1 to 4; preferably 2, 3 or 4; more preferably 2 or 3; still more preferably 2.

34. The conjugate of any one of items 32d to 32f or 33, wherein Z* is optionally substituted (C₁-C₈)alkyl.

34a. The conjugate of any one of items 32d to 32f, 33 or 34, wherein Y¹ is NR^{A20} or O, wherein R^{A20} is as defined in any one of the preceding claims;
preferably wherein Y¹ is NH or O;
more preferably wherein Y¹ is NH.

35. The conjugate of any one of the preceding items, wherein n is an integer ranging from 1 to 14, preferably from 2 to 14, more preferably from 3 to 14, still more preferably from 4 to 14, still more preferably from 5 to 12, still more preferably from 6 to 12, still more preferably from 7 to 10, even more preferably 8.

36. The conjugate of any one of items 1 to 34, wherein n is an integer ranging from 1 to 14, preferably from 1 to 12, more preferably from 2 to 10, still more preferably from 2 to 8, still more preferably from 2 to 6, still more preferably from 3 to 5, even more preferably 4.

37. The conjugate of any one of the preceding items, wherein the receptor binding molecule is selected from the group consisting of an antibody, an antibody fragment, a proteinaceous binding molecule with antibody-like binding properties, an aptamer, and a small molecule.

38. The conjugate of item 37, wherein the receptor binding molecule is an antibody; preferably wherein the antibody is selected from the group consisting of a monoclonal antibody, a chimeric antibody, a humanized antibody, a human antibody, and a single domain antibody, such as a camelid or shark single domain antibody.

39. The conjugate of item 38, wherein the receptor binding molecule is an antibody fragment;
preferably wherein the antibody fragment is a divalent antibody fragment, more preferably wherein the divalent antibody fragment is selected from the group consisting of a (Fab)₂′-fragment, a divalent single-chain Fv fragment, a dual affinity re-targeting (DART) antibody, and a diabody; or
preferably wherein the antibody fragment is a monovalent antibody fragment, more preferably wherein the monovalent antibody fragment is selected from the group consisting of a Fab fragment, a Fv fragment, and a single-chain Fv fragment (scFv).

40. The conjugate of item 37, wherein the receptor binding molecule is a proteinaceous binding molecule with antibody-like binding properties;
preferably wherein the proteinaceous binding molecule with antibody-like binding properties is selected from the group consisting of a mutein based on a polypeptide of the lipocalin family, a glubody, a protein based on the ankyrin scaffold, a protein based on the crystalline scaffold, an adnectin, an avimer, a DARPin, and an affibody.

41. The conjugate of any one of the preceding items, wherein X is O.

41a. The conjugate of any one of the preceding items 1 to 40, wherein X is NH.

42. The conjugate of any one of the preceding items, in particular of item 41, wherein the moiety X-D is derived from an aliphatic or aromatic alcohol.

43. The conjugate of any one of the preceding claims, wherein the drug moiety is selected from the group consisting of a Mitotic Spindle-Inhibitor such as (−)-Epipodophyllotoxin, a Dehydrogenase A-Inhibitor such as (R)-GNE-140, a Kinase-Inhibitor such as (S)-3-Hydroxy Midostaurin and (R)-3-Hydroxy Midostaurin, a BET-Inhibitor such as ABBV-744, a Estrogene Receptor Agonist such as Acolbifene, a Wee1-Inhibitor such as Adavosertib, a HSP90-Inhibitor such as Alvespimycin, a Kinase-Inhibitor such as ARS-1620, a FGFR-Inhibitor such as ASP5878, a MCT1-Inhibitor such as AZD3965, a mTOR-Inhibitor such as AZD-8055, a Kinase-Inhibitor such as Belizatinib, a HIF-2a inhibitor such as Belzutifan, a BCL-Inhibitor such as BM-1197, a VEGFR-Inhibitor such as Brivanib, a STAT3-Inhibitor such as C188, a anti tumor such as CB1151, a Kinase-Inhibitor such as Dasatinib, a EGFR-Inhibitor such as DBPR112, a CDK-Inhibitor such as Dinaciclib, a TRPC4 and TRCP5 Channel Activator such as Englerin A, a PRMT-Inhibitor such as EPZ015666, a Topoisomerase-Inhibitor such as Etoposide, a mTOR-Inhibitor such as Everolimus, a Methyltransferase-Inhibitor such as EZM 2302, a CDK-Inhibitor such as Fadraciclib, a USP7-Inhibitor such as FT671, a Estrogene Receptor Agonist such as Fulvestrant, a Estrogene Receptor Agonist such as Fulvestrant, a HSP90-Inhibitor such as Geldanamycin, a Estrogene Receptor Agonist such as GNE-274, a Kinase-Inhibitor such as GNE-493, a PRMT-Inhibitor such as GSK3326595, a Kinase-Inhibitor such as Hypothemycin, a CDK-Inhibitor such as IIIM-290, a DNA alkylator such as Illudin S, a Kinase-Inhibitor such as Ilorasertib, a Kinase-Inhibitor such as Larotrectinib, a Kinase-Inhibitor such as Larotrectinib, a IGF-1-Inhibitor such as Linsitinib, a PRMT-Inhibitor such as LLY-283, a HSP90-Inhibitor such as Luminespib, a FGFR-Inhibitor such as LY2874455, a Kinase-Inhibitor such as Mirdametinib, a Kinase-Inhibitor such as MRTX1133, a Kinase-Inhibitor such as MRTX1133, a Kinase-Inhibitor such as Ningetinib, DNA minor groove binder such as Lurbinectidin or Trabectidin, a HSP90-Inhibitor such as NMS-E973, a Ribonucleotide Reductase-Inhibitor such as NSAH, a PLK1-Inhibitor such as Onvansertib, a mTOR-Inhibitor such as Palomid 529, a Kinase-Inhibitor such as PD166326, a NEDD8-Inhibitor such as Pevonedistat, a Kinase-Inhibitor such as PF-04217903, a Kinase-Inhibitor such as PF-06843195, a HSP90-Inhibitor such as PI-103, a Methyltransferase-Inhibitor such as Pinometostat, a Topoisomerase-Inhibitor such as PNU-159682, a Topoisomerase-Inhibitor such as Podofilox, a HDAC-Inhibitor such as QTX125, a mTOR-Inhibitor such as Rapamycin, a Tankyrase-Inhibitor such as RK-287107, a Kinase-Inhibitor such as RO4987655, a Kinase-Inhibitor such as RP-3500, a BCL-Inhibitor such as S55746, a BCL-Inhibitor such as S65487, a EGFR-Inhibitor such as SDZ281-977, a Kinase-Inhibitor such as SU14813, a Kinase-Inhibitor such as TC-A 2317, a Kinase-Inhibitor such as Teleocidin A1, a Ribonucleotide Reductase-Inhibitor such as Tezacitabine, a Kinase-Inhibitor such as TG 100572, a Inhibitor of RNA splicing such as Thailanstatin A, a Kinase-Inhibitor such as UNC5293, a Kinase-Inhibitor such as UNC5293, a HSP90-Inhibitor such as VER-50589, a eIF4A-Inhibitor such as Zotatifin and analogues or prodrugs thereof.

43a. The conjugate of any one of the preceding claims, wherein the drug moiety is selected from the group consisting of camptothecin compounds, TOPK inhibitors, CDK inhibitors, bromodomain inhibitors, HSP70 inhibitors, HSP90 inhibitors, ribonucleotide reductase inhibitors, Aurora B kinase inhibitors, auristatins, maytansinoids, calicheamycins, tubulysins, amatoxins, dolastatins, pyrrolobenzodiazepine dimers, indolino-benzodiazepine dimers, radioisotopes, therapeutic proteins and peptides (or fragments thereof), KSP inhibitors, eukaryotic Translation Initiation Factor 4E (eIF4E) inhibitors, nicotinamide phosphoribosyltransferase (Nampt) inhibitors, dihydroorotate dehydrogenase (DHODH) inhibitors, taxanes, and analogues or prodrugs thereof.

44. The conjugate of item 43 or 43a, wherein the drug moiety is a camptothecin compound.

45. The conjugate of item 44, wherein the camptothecin compound is selected from the group consisting of DXD, SN38, exatecan, camptothecin, topotecan, irinotecan, belotecan, lurtotecan, rubitecan, silatecan, cositecan, and gimatecan.

46. The conjugate of item 45, wherein the camptothecin compound is DXD, SN38 or exatecan, preferably DXD or SN38.

46a. The conjugate of item 43 or 43a, wherein the drug moiety is a TOPK inhibitor.

46b. The conjugate of item 46a, wherein the TOPK inhibitor is OTS-964.

46c. The conjugate of item 43 or 43a, wherein the drug moiety is a CDK inhibitor.

46d. The conjugate of item 46c, wherein the CDK inhibitor is ganetespib or Roniciclib.

46e. The conjugate of item 43 or 43a, wherein the drug moiety is a bromodomain inhibitor.

46f. The conjugate of item 46e, wherein the bromodomain inhibitor is birabresib.

46g. The conjugate of item 43 or 43a, wherein the drug moiety is a HSP90 inhibitor.

46h. The conjugate of item 46g, wherein the HSP90 inhibitor is SNX-2112.

46i. The conjugate of item 43 or 43a, wherein the drug moiety is a ribonucleotide reductase inhibitor.

46j. The conjugate of item 46i, wherein the ribonucleotide reductase inhibitor is gemcitabine.

46k. The conjugate of item 46j, wherein the drug moiety is an Aurora B kinase inhibitor.

461. The conjugate of item 46k, wherein the Aurora B kinase inhibitor is barasertib.

46m. The conjugate of item 43 or 43a, wherein the drug moiety is a HSP70 inhibitor.

46n. The conjugate of item 46m, wherein the HSp 70 inhibitor is Triptolide.

46o. The conjugate of item 43 or 43a, wherein the drug moiety is a nicotinamide phosphoribosyltransferase (Nampt) inhibitor.

46p. The conjugate of item 460, wherein the Nampt inhibitor is Nampt-IN-1,

46q. The conjugate of item 43 or 43a, wherein the drug moiety is an eukaryotic Translation Initiation Factor 4E (eIF4E) inhibitor

46r. The conjugate of item 46q, wherein the eIF4E inhibitor is ON-013100.

46s. The conjugate of item 43 or 43a, wherein the drug moiety is a dihydroorotate dehydrogenase (DHODH) inhibitor.

46t. The conjugate of item 46s, wherein the DHODH inhibitor is Bay-2402234 or DHODH-IN-16.

46u. The conjugate of item 43 or 43a, wherein the drug moiety is a taxane.

46v. The conjugate of item 46u, wherein the taxane is Paclitaxel, albumin-bound Paclitaxel (nab-Paclitaxel), Docetaxel, Cabazitaxel or Abraxan.

47. The conjugate of item 43 or 43a, wherein the drug moiety is an auristatin.

48. The conjugate of item 47, wherein the drug moiety is monomethyl auristatin E (MMAE) or monomethyl auristatin F (MMAF).

49. The conjugate of any one of the preceding items, wherein M is O or NH.

49a. The conjugate of item 49, wherein M is O.

50. The conjugate of item 49, wherein M is O, X is O and Y¹ is NR^{A20}, wherein R^{A20} is as defined in any one of the preceding items;
preferably wherein M is O, X is O and Y¹ is NH;
preferably wherein m is 0.

50a. The conjugate of item 49a, wherein M is O, X is NH and Y¹ is O;
preferably wherein m is 0.

50b. The conjugate of item 49, wherein M is NH.

50c. The conjugate of item 50b, wherein M is NH, X is O and Y¹ is O.

51. The conjugate of item 49 or 50, wherein the drug moiety is a camptothecin compound;
preferably wherein the camptothecin compound is SN38 or DXD;
more preferably wherein the camptothecin compound is SN38.

52. The conjugate of any one of the preceding items, wherein the linker L has the formula (L-I):

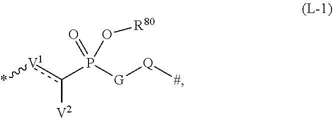

wherein:
[symbol] is a double bond; or
[symbol] is a bond;
V² is absent when [symbol] is a double bond; or
V² is selected from the group consisting of hydrogen, (C₁-C₈)alkyl, (C₆-C₁₀)aryl, and (C₁-C₈)alkylene(C₆-C₁₀)aryl when [symbol] is a bond;
V¹ is R^{V11—C} when [symbol] is a double bond; or
V¹

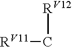

is when [symbol] is a bond;
G is NR^{G70}, S, O, or CR^{G71}R^{G72}
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl;
R⁸⁰ is an optionally substituted aliphatic residue or an optionally substituted aromatic residue;
* indicates the attachment to the receptor binding molecule (RBM); and
# indicates the attachment to M.

53. The conjugate of item 52, wherein [symbol] is a double bond; V² is absent; V¹ is R^{V11—C}; and R^{V11} is selected from the group consisting of hydrogen, (C₁-C₈)alkyl, (C₆-C₁₀)aryl, and (C₁-C₈)alkylene(C₆-C₁₀)aryl; preferably R^{V11} is hydrogen or (C₁-C₈)alkyl; more preferably R^{V11} is hydrogen.

54. The conjugate of item 52, wherein [symbol] is a bond; V² is selected from the group consisting of hydrogen, (C₁-C₈)alkyl, (C₆-C₁₀)aryl, and (C₁-C₈)alkylene(C₆-C₁₀)aryl; preferably, V² is hydrogen or (C₁-C₈)alkyl; more preferably, V² is hydrogen; V¹ is

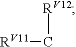

R^{V11} is selected from the group consisting of hydrogen, (C₁-C₈)alkyl, (C₆-C₁₀)aryl, and (C₁-C₈)alkylene(C₆-C₁₀)aryl; preferably R^{V11} is hydrogen or (C₁-C₈)alkyl, more preferably R^{V11} is hydrogen; R^{V12} is selected from the group consisting of hydrogen, (C₁-C₈)alkyl, (C₆-C₁₀)aryl, and (C₁-C₈)alkylene(C₆-C₁₀)aryl; preferably R^{V12} is hydrogen or (C₁-C₈)alkyl, more preferably R^{V12} is hydrogen.

55. The conjugate of any one of items 52 to 54, wherein G is NR^{G70}, wherein R^{G70} is as defined in any one of items 52 to 54;
preferably wherein G is NH.

56. The conjugate of any one of items 52 to 55, wherein Q is:

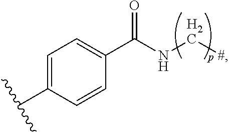

wherein:
p is an integer ranging from 2 to 20;
[symbol] indicates the attachment to G; and
# indicates the attachment to M.

56a. The conjugate of any one of items 52 to 55, wherein Q is

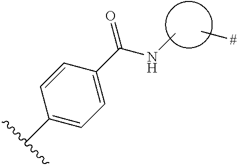

wherein [symbol] is a (C₃-C₈)carbocycle, (C₆-C₁₀)aryl (phenyl), a five- or six-membered heterocyclic ring comprising 1, 2 or 3 heteroatoms independently selected from the group consisting of N, O and S; preferably (C₃-C₈)cycloalkyl; more preferably 5-, 6-, or 7-membered cycloalkyl, even more preferably cyclohexyl;
# indicates the attachment to M; and
[symbol] indicates the attachment to G.

56b. The conjugate of item 56a, wherein [symbol] is cyclohexyl.

57. The conjugate of any one of items 52 to 56, wherein R⁸⁰ is a polyalkylene glycol unit.

58. The conjugate of item 57, wherein the polyalkylene glycol unit comprises 1 to 100 subunits having the structure:

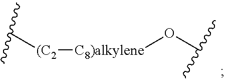

preferably wherein the polyalkylene glycol unit is:

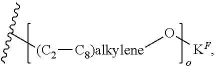

wherein:
[symbol] indicates the position of the O;
K^{F} is selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₈)alkyl and —(C₂-C₁₀)alkyl-N((C₁-C₈)alkyl)₂; preferably K^{F} is H; and
o is an integer ranging from 1 to 100.

59. The conjugate of item 57 or 58, wherein R⁸⁰ is a polyethylene glycol unit.

60. The conjugate of item 59, wherein the polyethylene glycol unit comprises 1 to 100 subunits having the structure:

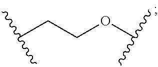 [SMILES: *CCO*]

preferably wherein the polyethylene glycol unit is:

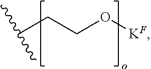

wherein:
[symbol] indicates the position of the O;
K^{F} is selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₈)alkyl and —(C₂-C₁₀)alkyl-N((C₁-C₈)alkyl)₂; preferably K^{F} is H; and
o is an integer ranging from 1 to 100.

61. The conjugate of any one of items 52 to 60, wherein attachment to the receptor binding molecule (RBM) is via a sulfur atom.

62. The conjugate of item 61, wherein the conjugate has formula (Ia2):

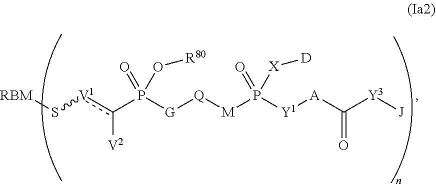

or a pharmaceutically acceptable salt or solvate thereof;
wherein RBM, V¹, V², [symbol] , R⁸⁰, G, Q, M, X, D, Y¹, A, Y³, J and n are as defined in any one of the preceding items.

63. A conjugate having the formula (Ia):

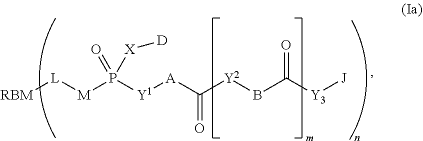

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

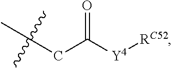

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl; and
n is an integer ranging from 1 to 20.

64. The conjugate of item 63, having formula (Ia1):

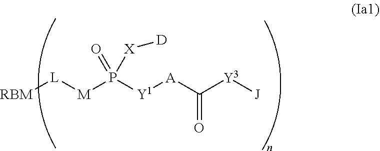

or a pharmaceutically acceptable salt or solvate thereof;
wherein RBM, L, M, X, D, Y¹, A, Y³, J and n are as defined in item 63.

65. The conjugate of item 64, wherein the conjugate has formula (Ia2):

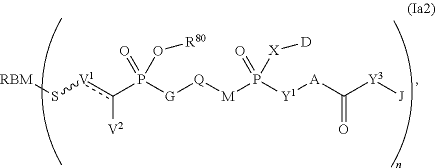

or a pharmaceutically acceptable salt or solvate thereof;
wherein RBM, M, X, D, Y¹, A, Y³, J and n are as defined in item 63 or 64;
[symbol] is a double bond; or
[symbol] is a bond;
V² is absent when [symbol] is a double bond; or
V² is selected from the group consisting of hydrogen, (C₁-C₈)alkyl, (C₆-C₁₀)aryl, and (C₁-C₈)alkylene(C₆-C₁₀)aryl when [symbol] is a bond;
V¹ is R^{V11—C} when [symbol] is a double bond; or
V¹ is

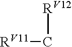

when [symbol] is a bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl; and
R⁸⁰ is an optionally substituted aliphatic residue or an optionally substituted aromatic residue.

66. The conjugate of any one of items 63 to 65, wherein RBM, L, V¹, V², [symbol] , R⁸⁰, G, Q, M, X, D, Y¹, A, Y², B, Y³, J, m and n are as defined in any one of the preceding items.

67. A conjugate having the formula (Ib1):

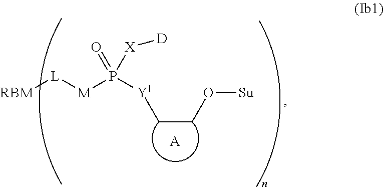

or a pharmaceutically acceptable salt or solvate thereof;
wherein:
RBM is a receptor binding molecule;
L is a linker;
M is O, NR^{M60}, or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;

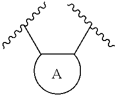

is an optionally substituted four- to seven-membered, preferably five- or six-membered, carbocyclic or heterocyclic ring;
[symbol] indicate the positions of the oxygen atom and Y¹; preferably the attachment points to the oxygen atom and Y¹ are two adjacent atoms of the ring;
Su is a sugar moiety; and
n is an integer ranging from 1 to 20.

68. The conjugate of item 67, wherein RBM, L, M, X, D, Y¹,

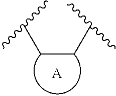

[symbol]

, Su and n are as defined in any one of the preceding items.

69. A conjugate having the formula (Ic1):

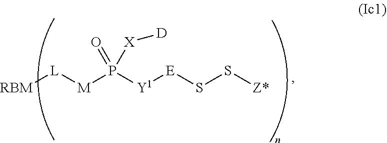

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

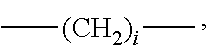

which can be optionally substituted; and wherein i is an integer ranging from 1 to 4; preferably 2, 3 or 4; more preferably 2 or 3; still more preferably 2;
Z* is an optionally substituted aliphatic residue or an optionally substituted aromatic residue; and
n is an integer ranging from 1 to 20.

70. The conjugate of item 69, wherein RBM, L, M, X, D, Y¹, E, i, Z* and n are as defined in any one of the preceding items.

70a. A conjugate having the formula (Id1):

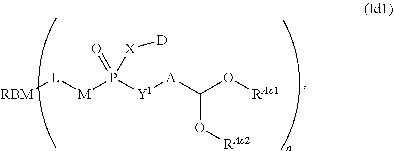

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
R^{Ac1} and R^{Ac2} are each independently an optionally substituted aliphatic residue or an optionally substituted aromatic residue; optionally, R^{Ac1} and R^{Ac2} can together with the oxygen atoms and the carbon atom form a 3- to 8-membered ring; and
n is an integer ranging from 1 to 20.

70b. The conjugate of item 70a, wherein RBM, L, M, X, D, Y¹, A, R^{Ac1}, R^{Ac2} and n are as defined in any one of the preceding items.

70c. A conjugate having the formula (Ie1):

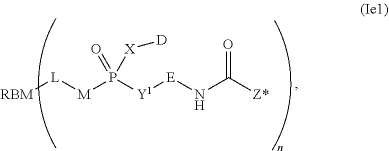

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

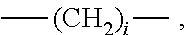

which can be optionally substituted; and wherein i is an integer ranging from 1 to 4; preferably 2, 3 or 4; more preferably 2 or 3; still more preferably 2;
Z* is an optionally substituted aliphatic residue or an optionally substituted aromatic residue; and
n is an integer ranging from 1 to 20.

70d. The conjugate of item 70c, wherein RBM, L, M, X, D, Y¹, E, i, Z* and n are as defined in any one of the preceding items.

70e. A conjugate having the formula (If1):

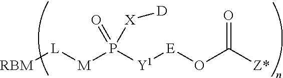

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

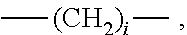

which can be optionally substituted; and wherein i is an integer ranging from 1 to 4; preferably 2, 3 or 4; more preferably 2 or 3; still more preferably 2;
Z* is an optionally substituted aliphatic residue or an optionally substituted aromatic residue; and
n is an integer ranging from 1 to 20.

70f. The conjugate of item 70e, wherein RBM, L, M, X, D, Y¹, E, i, Z* and n are as defined in any one of the preceding items.

71. A compound having the formula (II):

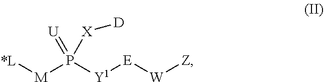

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A}21 and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group; and
W is a moiety which, after cleavage of the group Z, is capable of forming a ring together with the spacer E, Y¹ and the phosphorus atom.

71a. The compound of item 71, wherein W is a moiety which, after cleavage of the group Z, is capable of forming a four- to seven-membered ring together with the spacer E, Y¹ and the phosphorus atom.

71b. The compound of item 71a, wherein W is a moiety which, after cleavage of the group Z, is capable of forming a five- or six-membered ring together with the spacer E, Y¹ and the phosphorus atom.

72. The compound of any one of items 71 to 71b, having formula (IIa):

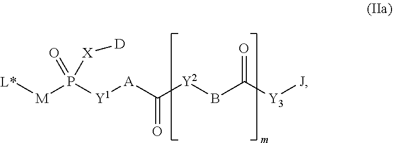

or a pharmaceutically acceptable salt or solvate thereof;
wherein:
L*, M, X, D and Y¹ are as defined in item 71;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₃)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₃)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

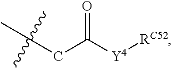

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl.

73. The compound of item 72, having formula (IIa1):

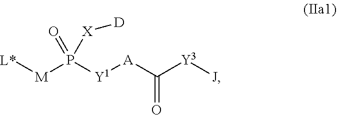

or a pharmaceutically acceptable salt or solvate thereof;
wherein L*, M, X, D, Y¹, A, Y³ and J are as defined in item 72.

74. The compound of item 73, having formula (IIa2):

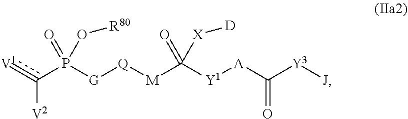

or a pharmaceutically acceptable salt or solvate thereof;
wherein M, X, D, Y¹, A, Y³ and J are as defined in item 72 or 73;
[symbol] is a triple bond; or
[symbol] is a double bond;
V² is absent when [symbol] is a triple bond; or
V² is selected from the group consisting of hydrogen, (C₁-C₈)alkyl, (C₆-C₁₀)aryl, and (C₁-C₈)alkylene(C₆-C₁₀)aryl when [symbol] is a double bond;
V¹ is R^{V11—C} when [symbol] is a triple bond; or
V¹ is

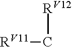

when [symbol] is a double bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl; and
R⁸⁰ is an optionally substituted aliphatic residue or an optionally substituted aromatic residue.

75. The compound of any one of items 72 to 74, wherein L*, V¹, V², [symbol] , R⁸⁰, G, Q, M, X, D, Y¹, A, Y², B, Y³, J and m are as defined in any one of the preceding items.

76. The compound of any one of items 71 to 71b, having the formula (IIb):

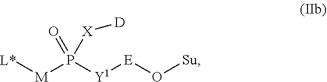

or a pharmaceutically acceptable salt or solvate thereof;
wherein:
L*, M, X, D and Y¹ are as defined in item 71;
E is a spacer; and
Su is a sugar moiety which is bound to the oxygen atom via a cleavable bond

76a. The compound of item 76, wherein the sugar moiety Su is protected or unprotected.

77. The compound of item 76 or item 76a, having the formula (IIb1):

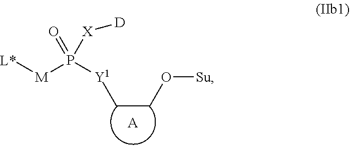

or a pharmaceutically acceptable salt or solvate thereof;
wherein:
L*, M, X, D, Y¹ and Su are as defined in item 76;

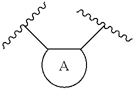

is an optionally substituted four- to seven-membered, preferably five- or six-membered, carbocyclic or heterocyclic ring; and
[symbol] indicate the positions of the oxygen atom and Y¹; preferably the attachment points to the oxygen atom and Y¹ are two adjacent atoms of the ring.

78. The compound of item 76, 76a or 77, wherein L*, M, X, D, Y¹,

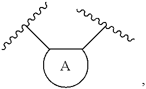

[symbol]

, E and Su are as defined in any one of the preceding items.

79. The compound of any one of items 71 to 71b, having the formula (IIc):

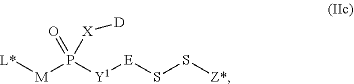

or a pharmaceutically acceptable salt or solvate thereof;
wherein:
L*, M, X, D and Y¹ are as defined in item 71;
E is a spacer;
Z* is an optionally substituted aliphatic residue or an optionally substituted aromatic residue; and
wherein the sulfur bound to the spacer E, after cleavage of the disulfide bond, is capable of forming a ring together with the spacer E, Y¹ and the phosphorus atom.

80. The compound of item 79 having the formula (IIc1):

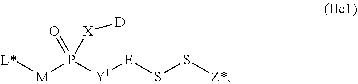

or a pharmaceutically acceptable salt or solvate thereof;
wherein:
L*, M, X, D, Y¹ and Z* are as defined in item 79; and
E is

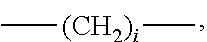

which can be optionally substituted; and wherein i is an integer ranging from 1 to 4; preferably 2, 3 or 4; more preferably 2 or 3; still more preferably 2.

81. The compound of item 79 or 80, wherein L*, M, X, D, Y¹, E, i and Z* are as defined in any one of the preceding items.

81a. The compound of item 71 having the formula (IId):

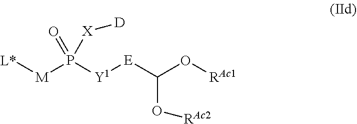

or a pharmaceutically acceptable salt or solvate thereof;
wherein:
L*, M, X, D, Y¹ and E are as defined in item 71; and
R^{Ac1} and R^{Ac}2 are each independently an optionally substituted aliphatic residue or an optionally substituted aromatic residue; optionally, R^{Ac1} and R^{Ac2} can together with the oxygen atoms and the carbon atom form a 3- to 8-membered ring.

81b. The compound of item 81a having the formula (IId1):

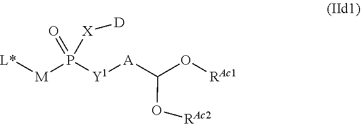

or a pharmaceutically acceptable salt or solvate thereof;
wherein:
L*, M, X, D, Y¹, R^{Ac1} and R^{Ac2} are as defined in item 81a; and
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring.

81c. The compound of item 81a or 81b, wherein RBM, L, M, X, D, Y¹, E, A, R^{Ac1} and R^{Ac2} are as defined in any one of the preceding items.

81d. The compound of item 71 having the formula (IIe):

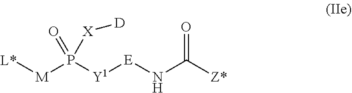

or a pharmaceutically acceptable salt or solvate thereof;
wherein:
L*, M, X, D, Y¹ and E are as defined in item 71; and
Z* is an optionally substituted aliphatic residue or an optionally substituted aromatic residue.

81e. The compound of item 81d having the formula (IIe1):

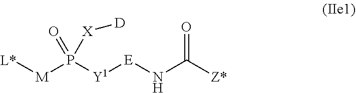

or a pharmaceutically acceptable salt or solvate thereof;
wherein:
L*, M, X, D, Y¹ and Z* are as defined in item 81d; and
E is

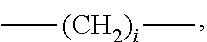

which can be optionally substituted; and wherein i is an integer ranging from 1 to 4; preferably 2, 3 or 4; more preferably 2 or 3; still more preferably 2.

81f. The compound of item 81d or 81e, wherein L*, M, X, D, Y¹, E, i and Z* are as defined in any one of the preceding items.

81g. The compound of item 71 having the formula (IIf):

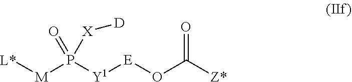

or a pharmaceutically acceptable salt or solvate thereof;
wherein L*, M, X, D, Y¹ and E are as defined in item 71; and
Z* is an optionally substituted aliphatic residue or an optionally substituted aromatic residue.

81h. The compound of item 81g having the formula (IIf1):

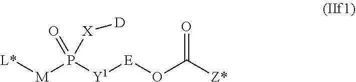

or a pharmaceutically acceptable salt or solvate thereof;
wherein L*, M, X, D, Y¹, and Z* are as defined in item 81g;
and
E is

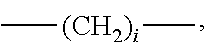

which can be optionally substituted; and wherein i is an integer ranging from 1 to 4; preferably 2, 3 or 4; more preferably 2 or 3; still more preferably 2.

81i. The compound of item 81g or 81h, wherein L*, M, X, D, Y¹, E, i and Z* are as defined in any one of the preceding items.

82. A compound having the formula (IIa):

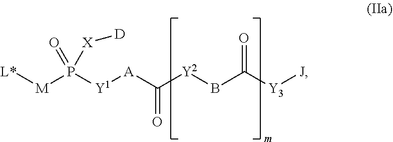

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

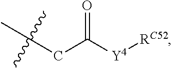

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl.

83. The compound of item 82, having formula (IIa1):

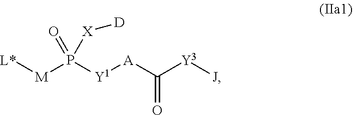

or a pharmaceutically acceptable salt or solvate thereof;
wherein L*, M, X, D, Y¹, A, Y³ and J are as defined in item 82.

84. The compound of item 83, having formula (IIa2):

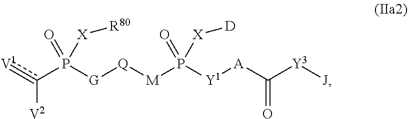

or a pharmaceutically acceptable salt or solvate thereof;
wherein M, X, D, Y¹, A, Y³ and J are as defined in item 82 or 83;
[symbol] is a triple bond; or
[symbol] is a double bond;
V² is absent when [symbol] is a triple bond; or
V² is selected from the group consisting of hydrogen, (C₁-C₈)alkyl, (C₆-C₁₀)aryl, and (C₁-C₈)alkylene(C₆-C₁₀)aryl when [symbol] is a double bond;
V¹ is R^{V11—C} when [symbol] is a triple bond; or
V¹ is

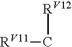

when [symbol] is a double bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl; and
R⁸⁰ is an optionally substituted aliphatic residue or an optionally substituted aromatic residue.

[symbol]

85. The compound of any one of items 82 to 84, wherein L*, V¹, V², [symbol] , R⁸⁰, G, Q, M, X, D, Y¹, A, Y², B, Y³, J and m are as defined in any one of the preceding items.

86. A compound having the formula (IIb1):

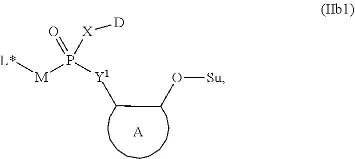

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;

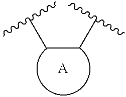

is an optionally substituted four- to seven-membered, preferably five- or six-membered, carbocyclic or heterocyclic ring; and
[symbol] indicate the positions of the oxygen atom and Y¹; preferably the attachment points to the oxygen atom and Y¹ are two adjacent atoms of the ring; and
Su is a sugar moiety.

87. The compound of item 86, wherein L*, M, X, D, Y¹,

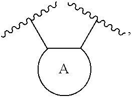

[symbol]

, and Su are as defined in any one of the preceding items.

88. A compound having the formula (IIc1):

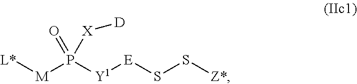

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

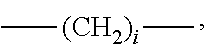

which can be optionally substituted; and wherein i is an integer ranging from 1 to 4; preferably 2, 3 or 4; more preferably 2 or 3; still more preferably 2; and
Z* is an optionally substituted aliphatic residue or an optionally substituted aromatic residue.

89. The compound of item 88, wherein L*, M, X, D, Y¹, E, i and Z* are as defined in any one of the preceding items.

90. A method of preparing a conjugate of formula (I), said method comprising:
reacting a compound of formula (II)

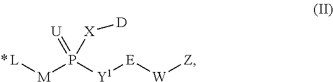

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group; and
W is a moiety which, after cleavage of the group Z, is capable of forming a ring together with the spacer E, Y¹ and the phosphorus;
with a receptor binding molecule (RBM) having a functional group reactive towards L* of a compound of formula (II), thereby forming a covalent bond between the receptor binding molecule (RBM) and the linker (L) to result in a conjugate of formula (I):

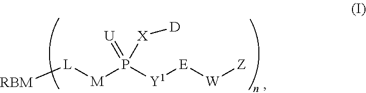

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group;
W is a moiety which, after cleavage of the group Z, is capable of forming a ring together with the spacer E, Y¹ and the phosphorus; and
n is an integer ranging from 1 to 20.

90a. The method of item 90, wherein W is a moiety which, after cleavage of the group Z, is capable of forming a four- to seven-membered ring together with the spacer E, Y¹ and the phosphorus.

90b. The method of item 90a, wherein W is a moiety which, after cleavage of the group Z, is capable of forming a five- or six-membered ring together with the spacer E, Y¹ and the phosphorus.

91. The method of any one of items 90 to 90b, said method comprising:
reacting a compound of formula (IIa)

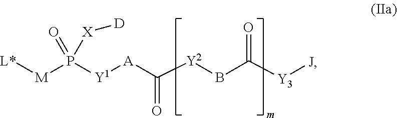

or a pharmaceutically acceptable salt or solvate thereof;
wherein:
L*, M, X, D and Y¹ are as defined in item 90;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

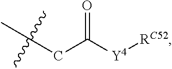

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl; with a receptor binding molecule (RBM) having a functional group reactive towards L* of a compound of formula (IIa), thereby forming a covalent bond between the receptor binding molecule (RBM) and the linker (L) to result in a conjugate of formula (Ia)

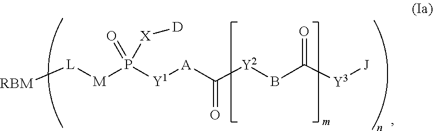

or a pharmaceutically acceptable salt or solvate thereof;
wherein:
RBM, L, M, X, D, Y¹ and n are as defined in item 90;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

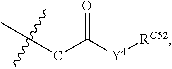

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring; or
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl.

92. The method of item 91, said method comprising:
Reacting a compound of formula (IIa1)

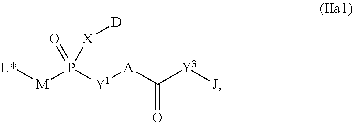

or a pharmaceutically acceptable salt or solvate thereof;
wherein L*, M, X, D, Y¹, A, Y³ and J are as defined in item 91;
with a receptor binding molecule (RBM) having a functional group reactive towards L* of a compound of formula (IIa1), thereby forming a covalent bond between the receptor binding molecule (RBM) and the linker (L) to result in a conjugate of formula (Ia1)

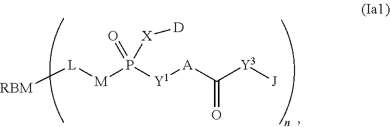

or a pharmaceutically acceptable salt or solvate thereof;
wherein:
RBM, L, M, X, D, Y¹ A, Y³, J and n are as defined in item 91.

93. The method of item 92, said method comprising:
Reacting a compound of formula (IIa2):

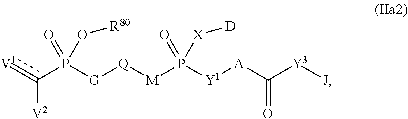

or a pharmaceutically acceptable salt or solvate thereof;
wherein M, X, D, Y¹, A, Y³ and J are as defined in item 91 or 92;
[symbol] is a triple bond; or
[symbol] is a double bond;
V² is absent when [symbol] is a triple bond; or
V² is selected from the group consisting of hydrogen, (C₁-C₈)alkyl, (C₆-C₁₀)aryl, and (C₁-C₈)alkylene(C₆-C₁₀)aryl when [symbol] is a double bond;
V¹ is R^{V11—C} when [symbol] is a triple bond; or
V¹ is

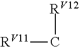

when [symbol] is a double bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl; and
R⁸⁰ is an optionally substituted aliphatic residue or an optionally substituted aromatic residue with a thiol-containing molecule of formula (III)

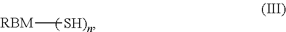

wherein RBM is a receptor binding molecule; and
n is an integer ranging from 1 to 20;
to result in a conjugate of formula (Ia2)

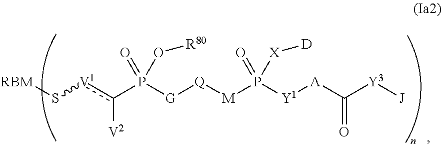

or a pharmaceutically acceptable salt or solvate thereof;
wherein:
RBM, M, X, D, Y¹, A, Y³, J and n are as defined in item 91 or 92;
[symbol] is a double bond when [symbol] in a compound of formula (IIa2) is a triple bond; or
[symbol] is a bond when [symbol] in a compound of formula (IIa2) is a double bond;
V² is absent when [symbol] is a double bond; or
V² is selected from the group consisting of hydrogen, (C₁-C₈)alkyl, (C₆-C₁₀)aryl, and (C₁-C₈)alkylene(C₆-C₁₀)aryl when [symbol] is a bond;
V¹ is R^{V11—C} when [symbol] is a double bond; or
V¹ is

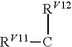

when [symbol] is a bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl; and
R⁸⁰ is an optionally substituted aliphatic residue or an optionally substituted aromatic residue.

94. The method of any one of items 91 to 93, wherein RBM, L, L*, V¹, V², [symbol] , [symbol] , R⁸⁰, G, Q, M, X, D, Y¹, A, Y², B, Y³, J, m and n are as defined in any one of the preceding items.

95. The method of any one of items 90 to 90b, comprising:
reacting a compound of formula (IIb)

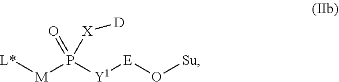

or a pharmaceutically acceptable salt or solvate thereof;
wherein:
L*, M, X, D and Y¹ are as defined in item 90;
E is a spacer;
Su is a sugar moiety which is bound to the oxygen atom via a cleavable bond; and wherein the oxygen, after cleavage of the sugar moiety Su, is capable of forming a ring together with the spacer E, Y¹ and the phosphorus;
with a receptor binding molecule (RBM) having a functional group reactive towards L* of a compound of formula (IIb), thereby forming a covalent bond between the receptor binding molecule (RBM) and the linker (L) to result in a conjugate of formula (Ib)

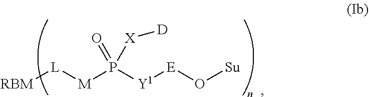

or a pharmaceutically acceptable salt or solvate thereof;
wherein:
RBM, L, M, X, D, Y¹ and n are as defined in item 90;
E is a spacer;
Su is a sugar moiety which is bound to the oxygen atom via a cleavable bond; and wherein the oxygen, after cleavage of the sugar moiety Su, is capable of forming a ring together with the spacer E, Y¹ and the phosphorus.

96. The method of item 95, said method comprising:
Reacting a compound of formula (IIb1):

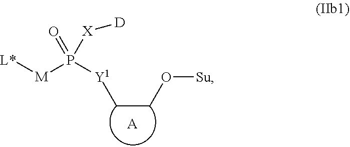

or a pharmaceutically acceptable salt or solvate thereof;
wherein:
L*, M, X, D, Y¹ and Su are as defined in item 95;

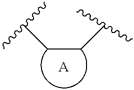

is an optionally substituted four- to seven-membered, preferably five- or six-membered, carbocyclic or heterocyclic ring; and
[symbol] indicate the positions of the oxygen atom and Y¹; preferably the attachment points to the oxygen atom and Y¹ are two adjacent atoms of the ring
with a receptor binding molecule (RBM) having a functional group reactive towards L* of a compound of formula (IIb1), thereby forming a covalent bond between the receptor binding molecule (RBM) and the linker (L) to result in a conjugate of formula (Ib1)

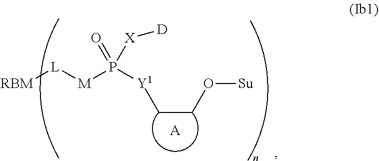

or a pharmaceutically acceptable salt or solvate thereof;
wherein:
RBM, L, M, X, D, Y¹, Su and n are as defined in item 95;

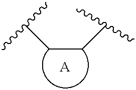

is an optionally substituted four- to seven-membered, preferably five- or six-membered, carbocyclic or heterocyclic ring; and
[symbol] indicate the positions of the oxygen atom and Y¹; preferably the attachment points to the oxygen atom and Y¹ are two adjacent atoms of the ring.

97. The method of item 95 or 96, wherein RBM, L, L*, M, X, D, Y¹,

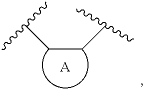

[symbol]

, E, Su and n are as defined in any one of the preceding items.

98. The method of any one of item 90 to 90b, comprising:
reacting a compound of formula (IIc)

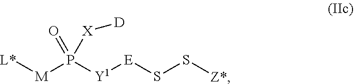

or a pharmaceutically acceptable salt or solvate thereof;
wherein:
L*, M, X, D and Y¹ are as defined in item 90;
E is a spacer; and
Z* is an optionally substituted aliphatic residue or an optionally substituted aromatic residue;
wherein the sulfur bound to the spacer E, after cleavage of the disulfide bond, is capable of forming a ring together with the spacer E, Y¹ and the phosphorus;
with a receptor binding molecule (RBM) having a functional group reactive towards L* of a compound of formula (IIc), thereby forming a covalent bond between the receptor binding molecule (RBM) and the linker (L) to result in a conjugate of formula (Ic)

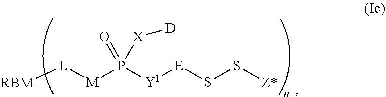

or a pharmaceutically acceptable salt or solvate thereof;
wherein:
RBM, L, M, X, D, Y¹ and n are as defined in item 90;
E is a spacer; and
Z* is an optionally substituted aliphatic residue or an optionally substituted aromatic residue;
wherein the sulfur bound to the spacer E, after cleavage of the disulfide bond, is capable of forming a ring together with the spacer E, Y¹ and the phosphorus atom.

99. The method of item 98, comprising:
Reacting a compound of formula (IIc1)

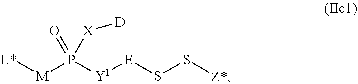

or a pharmaceutically acceptable salt or solvate thereof;
wherein L*, M, X, D, Y¹ and Z* are as defined in item 98; and
E is

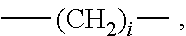

which can be optionally substituted; and wherein i is an integer ranging from 1 to 4; preferably 2, 3 or 4; more preferably 2 or 3; still more preferably 2
with a receptor binding molecule (RBM) having a functional group reactive towards L* of a compound of formula (IIc), thereby forming a covalent bond between the receptor binding molecule (RBM) and the linker (L) to result in a conjugate of formula (Ic1)

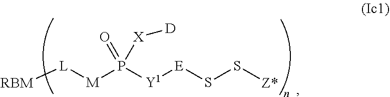

or a pharmaceutically acceptable salt or solvate thereof;
wherein:
RBM, L, M, X, D, Y¹, Z* and n are as defined in item 98; and
E is

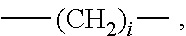

which can be optionally substituted; and wherein i is an integer ranging from 1 to 4; preferably 2, 3 or 4; more preferably 2 or 3; still more preferably 2.

100. The method of item 98 or 99, wherein RBM, L, L*, M, X, D, Y¹, E, i, Z* and n are as defined in any one of the preceding items.

100a. The method of any one of items 90 to 90b, comprising:
reacting a compound of formula (IId)

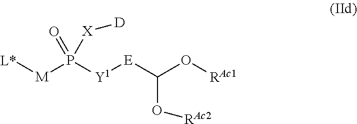

or a pharmaceutically acceptable salt or solvate thereof; wherein
L*, M, X, D, Y¹ and E are as defined in item 90; and
R^{Ac1} and R^{Ac2} are each independently an optionally substituted aliphatic residue or an optionally substituted aromatic residue; optionally, R^{Ac1} and R^{Ac2} can together with the oxygen atoms and the carbon atom form a 3- to 8-membered ring;
with a receptor binding molecule (RBM) having a functional group reactive towards L* of a compound of formula (IId), thereby forming a covalent bond between the receptor binding molecule (RBM) and the linker (L) to result in a conjugate of formula (Id)

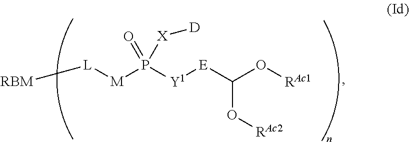

or a pharmaceutically acceptable salt or solvate thereof; wherein
RBM, L, M, X, D, Y¹, E, and n are as defined in item 90; and
R^{Ac1} and R^{Ac2} are each independently an optionally substituted aliphatic residue or an optionally substituted aromatic residue; optionally, R^{Ac1} and R^{Ac2} can together with the oxygen atoms and the carbon atom form a 3- to 8-membered ring.

100b. The method of item 100a, wherein RBM, L, L*, M, X, D, Y¹, E, R^{Ac1}, R^{Ac2} and n are as defined in any one of the preceding items.

100c. The method of any one of items 90 to 90b, comprising:
reacting a compound of formula (IIe):

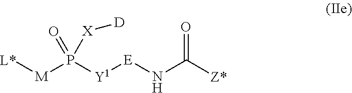

or a pharmaceutically acceptable salt or solvate thereof; wherein
L*, M, X, D, Y¹ and E are as defined in item 90; and
Z* is an optionally substituted aliphatic residue or an optionally substituted aromatic residue;
with a receptor binding molecule (RBM) having a functional group reactive towards L* of a compound of formula (IIe), thereby forming a covalent bond between the receptor binding molecule (RBM) and the linker (L) to result in a conjugate of formula (Ie)

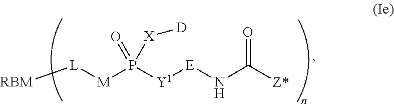

or a pharmaceutically acceptable salt or solvate thereof; wherein:
RBM, L, M, X, D, Y¹, E and n are as defined in item 90; and
Z* is an optionally substituted aliphatic residue or an optionally substituted aromatic residue.

100d. The method of item 100c, wherein RBM, L, L*, M, X, D, Y¹, E, i, Z* and n are as defined in any one of the preceding items.

100e. The method of any one of items 90 to 90b, comprising:
reacting a compound of formula (IIf)

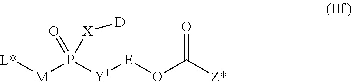

or a pharmaceutically acceptable salt or solvate thereof; wherein
L*, M, X, D, Y¹ and E are as defined in item 90; and
Z* is an optionally substituted aliphatic residue or an optionally substituted aromatic residue;
with a receptor binding molecule (RBM) having a functional group reactive towards L* of a compound of formula (IIe), thereby forming a covalent bond between the receptor binding molecule (RBM) and the linker (L) to result in a conjugate of formula (If)

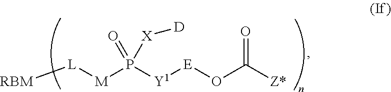

or a pharmaceutically acceptable salt or solvate thereof; wherein:
RBM, L, M, X, D, Y¹, E and n are as defined in item 90; and
Z* is an optionally substituted aliphatic residue or an optionally substituted aromatic residue.

100f. The method of item 100e, wherein RBM, L, L*, M, X, D, Y¹, E, i, Z* and n are as defined in any one of the preceding items.

101. A method of preparing a conjugate of formula (Ia), said method comprising:
reacting a compound of formula (IIa)

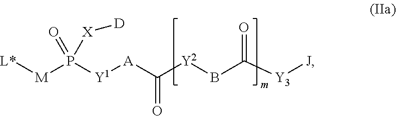

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

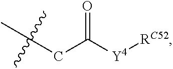

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl;
with a receptor binding molecule (RBM) having a functional group reactive towards L* of a compound of formula (IIa), thereby forming a covalent bond between the receptor binding molecule (RBM) and the linker (L) to result in a conjugate of formula (Ia)

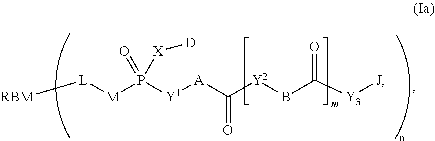

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is

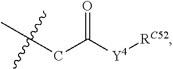

wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl; and
n is an integer ranging from 1 to 20.

102. The method of item 101, said method comprising:
Reacting a compound of formula (IIa1)

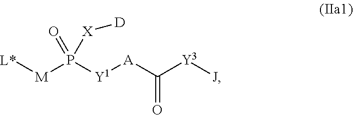

or a pharmaceutically acceptable salt or solvate thereof;
wherein L*, M, X, D, Y¹, A, Y³ and J are as defined in item 101;
with a receptor binding molecule (RBM) having a functional group reactive towards L* of a compound of formula (IIa1), thereby forming a covalent bond between the receptor binding molecule (RBM) and the linker (L) to result in a conjugate of formula (Ia1)

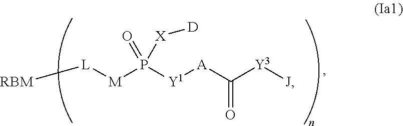

or a pharmaceutically acceptable salt or solvate thereof;
wherein:
RBM, L, M, X, D, Y¹ A, Y³, J and n are as defined in item 101.

103. The method of item 102, said method comprising:
Reacting a compound of formula (IIa2):

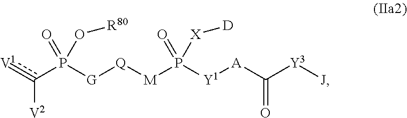

or a pharmaceutically acceptable salt or solvate thereof;
wherein M, X, D, Y¹, A, Y³ and J are as defined in item 101 or 102;
[symbol] is a triple bond; or
[symbol] is a double bond;
V² is absent when [symbol] is a triple bond; or
V² is selected from the group consisting of hydrogen, (C₁-C₈)alkyl, (C₆-C₁₀)aryl, and (C₁-C₈)alkylene(C₆-C₁₀)aryl when [symbol] is a double bond;
V¹ is R^{V11—C} when [symbol] is a triple bond; or
V¹ is

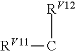

when [symbol] is a double bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl; and
R⁸⁰ is an optionally substituted aliphatic residue or an optionally substituted aromatic residue
with a thiol-containing molecule of formula (III)

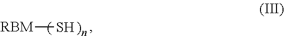

wherein RBM is a receptor binding molecule; and
n is an integer ranging from 1 to 20;
to result in a conjugate of formula (Ia2)

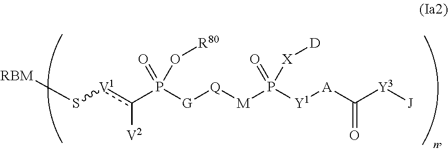

or a pharmaceutically acceptable salt or solvate thereof;
wherein:
RBM, M, X, D, Y¹, A, Y³, J and n are as defined in item 101 or 102;
[symbol] is a double bond when [symbol] in a compound of formula (IIa2) is a triple bond; or
[symbol] is a bond when [symbol] in a compound of formula (IIa2) is a double bond;
V² is absent when [symbol] is a double bond; or
V² is selected from the group consisting of hydrogen, (C₁-C₈)alkyl, (C₆-C₁₀)aryl, and (C₁-C₈)alkylene(C₆-C₁₀)aryl when [symbol] is a bond;
V¹ is R^{V11—C} when [symbol] is a double bond; or
V¹ is

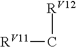

when [symbol] is a bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl; and
R⁸⁰ is an optionally substituted aliphatic residue or an optionally substituted aromatic residue.

104. The method of any one of items 101 to 103, wherein RBM, L, L*, V¹, V², [symbol] , [symbol] , R⁸⁰, G, Q, M, X, D, Y¹, A, Y², B, Y³, J, m and n are as defined in any one of the preceding items.

105. A method of preparing a conjugate of formula (Ib1), said method comprising:
Reacting a compound of formula (IIb1):

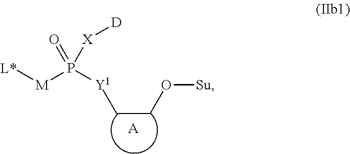

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;

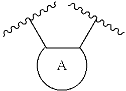

is an optionally substituted four- to seven-membered, preferably five- or six-membered, carbocyclic or heterocyclic ring; and
[symbol] indicate the positions of the oxygen atom and Y¹; preferably the attachment points to the oxygen atom and Y¹ are two adjacent atoms of the ring; and
Su is a sugar moiety
with a receptor binding molecule (RBM) having a functional group reactive towards L* of a compound of formula (IIb1), thereby forming a covalent bond between the receptor binding molecule (RBM) and the linker (L) to result in a conjugate of formula (Ib1)

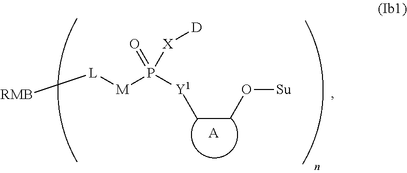

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;

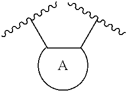

is an optionally substituted four- to seven-membered, preferably five- or six-membered, carbocyclic or heterocyclic ring;
[symbol] indicate the positions of the oxygen atom and Y¹; preferably the attachment points to the oxygen atom and Y¹ are two adjacent atoms of the ring;
Su is a sugar moiety; and
n is an integer ranging from 1 to 20.

106. The method of item 105, wherein RBM, L, L*, M, X, D, Y¹,

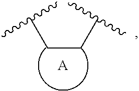

[symbol]

, Su and n are as defined in any one of the preceding items.

107. A method of preparing a conjugate of formula (Ic1), comprising: Reacting a compound of formula (Ic1)

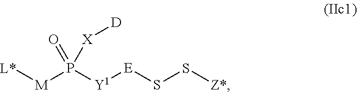

or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

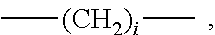

which can be optionally substituted; and wherein i is an integer ranging from 1 to 4; preferably 2, 3 or 4; more preferably 2 or 3; still more preferably 2; and
Z* is an optionally substituted aliphatic residue or an optionally substituted aromatic residue.
with a receptor binding molecule (RBM) having a functional group reactive towards L* of a compound of formula (IIc1), thereby forming a covalent bond between the receptor binding molecule (RBM) and the linker (L) to result in a conjugate of formula (Ic1)

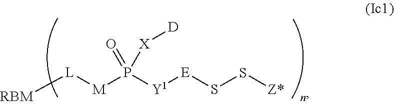

or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is

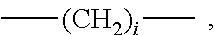

which can be optionally substituted; and wherein i is an integer ranging from 1 to 4; preferably 2, 3 or 4; more preferably 2 or 3; still more preferably 2;
Z* is an optionally substituted aliphatic residue or an optionally substituted aromatic residue; and
n is an integer ranging from 1 to 20.

108. The method of item 107, wherein RBM, L, L*, M, X, D, Y¹, E, i, Z* and n are as defined in any one of the preceding items.

109. A conjugate, or a pharmaceutically acceptable salt or solvate thereof, obtainable or being obtained by a method of any one of items 90 to 108.

110. A pharmaceutical composition comprising a conjugate of any one of items 1 to 70 and 109.

111. The pharmaceutical composition of item 110, wherein the pharmaceutical composition comprises a population of a conjugate of any one of items 1 to 70 and 109, and wherein the average number of drug moieties per receptor binding molecule in the composition is from more than 0 to about 14, preferably from about 1 to about 14, more preferably from about 2 to about 14, still more preferably from about 3 to about 14, still more preferably from about 4 to about 14, still more preferably from about 5 to about 12, still more preferably from about 6 to about 12, still more preferably from about 6 to about 10, even more preferably about 8.

112. The pharmaceutical composition of item 110, wherein the pharmaceutical composition comprises a population of a conjugate of any one of items 1 to 70 and 109, and wherein the average number of drug moieties per receptor binding molecule is from more than 0 to about 14, preferably from about 1 to about 14, more preferably from about 1 to about 12, still more preferably from about 2 to about 10, still more preferably from about 2 to about 8, still more preferably from about 2 to about 6, still more preferably from about 3 to about 5, even more preferably about 4.

113. A conjugate of any one of items 1 to 70 and 109 for use in a method of treating a disease.

113a. A conjugate of any one of items 1 to 70 and 109 for use in the manufacture of a medicament for treating a disease.

113b. A conjugate of any one of items 1 to 70 and 109 for use as a medicament.

114. The conjugate for use of item 113, 113a or 113b, wherein the disease is cancer.

115. A pharmaceutical composition of any one of items 110 to 112 for use in a method of treating a disease.

116. The pharmaceutical composition for use of item 115, wherein the disease is cancer.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number. For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

As used herein the terms “about”, “approximately” or “essentially” mean within 20%, preferably within 15%, preferably within 10%, and more preferably within 5% of a given value or range. It also includes the concrete number, i.e. “about 20” includes the number of 20.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### EXAMPLES

An even better understanding of the present invention and of its advantages will be evident from the following examples, offered for illustrative purposes only. The examples are not intended to limit the scope of the present invention in any way.

### General Information

### Chemicals, Solvents and Antibodies

Chemicals and solvents were purchased from Merck (Merck group, Germany), TCI (Tokyo chemical industry CO., LTD., Japan), Iris Biotech (Iris Biotech GmbH, Germany), MCE (MedChemExpress, USA) and Carl Roth (Carl Roth GmbH+Co. KG, Germany) and used without further purification. Dry solvents were purchased from Merck (Merck group, Germany). Trastuzumab was purchased from Roche (Hoffmann-La Roche A G, Switzerland). Enhertu was purchased from Daichi-Sankyo (Daiichi Sankyō K. K, Japan), and Trodelvy was purchased from Gilead sciences (Gilead incorporated, United states).

### Preparative HPLC

Preperative HPLC was performed on a BÜCHI Pure C-850 Flash-Prep system (BÜCHI Labortechnik A G, Switzerland) using a VP 250/10 Macherey-Nagel Nucleodur C18 HTec Spum column (Macherey-Nagel GmbH & Co. Kg, Germany) for smaller scales. The following gradients were used: Method C: A=H2O+0.1% TFA (trifluoroacetic acid), B=MeCN (acetonitrile)+0.1% TFA, flow rate 6 ml/min, 30% B 0-5 min, 30-70% B 5-35 min, 99% B 35-45 min. Method D: A=H2O, B=MeCN (acetonitrile), flow rate 6 ml/min, 30% B 0-5 min, 30-70% B 5-35 min, 99% B 35-45 min. For larger scales, a VP 250/21 Macherey-Nagel Nucleodur C18 HTec Spum column (Macherey-Nagel GmbH & Co. Kg, Germany) was used with the following gradients were used: Method E: A=H2O+0.1% TFA (trifluoroacetic acid), B=MeCN (acetonitrile)+0.1% TFA, flow rate 14 ml/min, 30% B 0-5 min, 30-70% B 5-35 min, 99% B 35-45 min. Large scales have been purified with a VP 250/32 Macherey-Nagel Nucleodur C18 HTec Spum column (Macherey-Nagel GmbH & Co. Kg, Germany) with the following gradients: Method F: A=H2O+0.1% TFA (trifluoroacetic acid), B=MeCN (acetonitrile)+0.1% TFA, flow rate 32 ml/min, 30% B 0-5 min, 30-90% B 5-35 min, 99% B 35-45 min.

### Hiqh Resolution LC/MS

Small molecules, linker-payloads, antibodies and ADCs were analyzed using a Waters H-class instrument equipped with a quaternary solvent manager, a Waters sample manager-FTN, a Waters PDA detector and a Waters column manager with an Acquity UPLC protein BEH C₄ column (300 Å, 1.7 μm, 2.1 mm×50 mm) for antibodies and ADCs. Here, samples were eluted at a column temperature of 80° C. The following gradient was used: A: 0.1% formic acid in H₂O; B: 0.1% formic acid in MeCN. 25% B 0-1 min, 0.4 mL/min, 25-95% B 1-3.5 min 0.2 mL/min, 95% B 3.5-4.5 min 0.2 mL/min, 95-25% B 4.5-5 min 0.4 mL/min, 25-95% B 5-5.5 min 0.4 mL/min, 95-25% B 5.5-7.5 min 0.4 mL/min. Mass analysis was conducted with a Waters XEVO G2-XS QTof analyzer. Proteins were ionized in positive ion mode applying a cone voltage of 40 kV. Raw data was analyzed with MaxEnt 1. Small molecules and linker-payloads were analyzed with an Acquity UPLC-BEH C18 column (300 Å, 1.7 μm,2.1 mm×50 mm). Here, samples were eluted at a column temperature of 45° C. with a flow rate of 0.4 mL/min. The following gradient was used: A: 0.1% formic acid in H₂O; B: 0.1% formic acid in MeCN. 2% B 0-1 min, 2-98% B 1-5 min, 98% B 5-5.5 min, 98-2% B 5.5-6 min, 2% B 6-7 min.

### Low Resolution LC/MS

Small molecules were analyzed on a Vanquish Flex UHPLC System with a DAD detector, Split Sampler FT (4° C.), Column Compartment H (45° C.) and binary pump F (Thermo Fisher Scientific, USA) using a Waters Acquity UPLC-CSH C18 column (130 Å, 1.7 μm, 2.1 mm×100 mm) with a flow rate of 0.4 mL/min. UV chromatograms were recorded at 220 or 254 nm. The following gradient was used: A: 0.1% formic acid in H₂O; B: 0.1% formic acid in MeCN. 2% B 0-1 min, 2-98% B 1-5 min, 98% B 5-6 min, 98-2% B 6-6.5 min.

### Antibody Expression

Palivizumab, Datopotamab, Brentuximab and Sacituzumab were transiently expressed in Expi-CHO—S cells (Thermo Fisher) by co-transfecting cells with pcDNA3.4 expression plasmids (Thermo Fisher), coding for the heavy and light chain of the respective sequences in a 1:1 ratio, using the Expi-CHO transfection system (Thermo Fisher).

Brentuximab: heavy chain (Fc-wild-type; SEQ ID NO: 2), light chain (SEQ ID NO: 3); Datopotamab: heavy chain (SEQ ID NO: 4), light chain (SEQ ID NO: 5); and Sacituzumab: heavy chain (SEQ ID NO: 6), light chain (SEQ ID NO: 7). Cells were harvested by centrifugation at 300 g for 5 minutes at 4° C. To clear micro particles from supernatant, supernatants were centrifuged at 4000-5000 g for 30 min at 4° C. For further clarification supernatants were passed through a 0.22 μm filter. Antibodies were purified from cleared and filtered supernatants via Protein A chromatography and analyzed by HPLC-SEC, HPLC-HIC, LC-MS and SDS-PAGE.

### Preparative Size-Exclusion-Chromatography

Protein purification by size-exclusion chromatography was conducted with an AKTA Pure FPLC system (GE Healthcare, United States) equipped with a F9-C-fraction collector.

### ADC Concentration Determination

The ADC concentrations were determined in a 96-well plate with a Pierce™ Rapid Gold BCA Protein Assay Kit (Thermo Fisher Scientific, USA) and a Bradford reagent B6916 (Merck, Germany) with pre-diluted protein assay standards of bovine gamma globulin (Thermo Fisher Scientific, USA). Results of both Assays were arithmetically averaged.

### Sample Preparation of ADCs and Antibodies for MS

0.5 μl PNGase-F solution (Pomega, Germany, Recombinant, cloned from Elizabethkingia miricola 10 u/μl) and 5 μL of a 100 mM solution of DTT in water were added to 50 μl of 0.2 mg/mL antibody or ADC in PBS and the solution was incubated at 37° C. for at least 2 hours. Samples were subjected to LC/MS, injecting 2 μl for each sample.

### Analytical Size-Exclusion Chromatography

Analytical size-exclusion chromatography (A-SEC) of the ADCs was conducted on a Vanquish Flex UHPLC System with a DAD detector, Split Sampler FT (4° C.), Column Compartment H (25° C.) and binary pump F (Thermo Fisher Scientific, USA) using a MAbPac SEC-1 300 Å, 4×300 mm column (Thermo Fisher Scientific, USA) with a flow rate of 0.15 mL/min. Separation of different ADC/mAb populations have been achieved during a 30 minute isocratic gradient using a phosphate buffer at pH 7 (20 mM Na2HPO4/NaH2PO4, 300 mM NaCl, 5% v/v isopropyl alcohol as a mobile phase. 8 μg ADC/mAb where loaded onto the column for A-SEC analysis. UV chromatograms were recorded at 220 and 280 nm.

### Analytical Hydrophobic Interaction Chromatography

The measurements were conducted on a Vanquish Flex UHPLC System (2.9) with a MabPac HIC Butyl 4.6×100 mm column (Thermo Fischer Scientific, USA). Separation of different ADCs/antibodies have been achieved with the following gradient: A: 1 M (NH4)2SO4, 500 mM NaCl, 100 mM NaH2PO4 pH 7.4 B: 20 mM NaH2PO4, 20% (v/v) Isopropyl alcohol, pH 7.4. 0% B: 0-1 min, 0-95% B: 1-15 min, 95% B: 15-20 min, 95-0% B: 20-23 min, 0% B: 23-25 min, with a flow of 700 uL/min. 15 μg sample where loaded onto the column for each analysis. UV chromatograms were recorded at 220 and 280 nm.

### General Method for the Conjugation of the Linker-Payload Constructs to Antibodies

50 μl of the antibody solution of 10.0 mg/ml in P5-conjugation buffer (50 mM Tris, 1 mM EDTA, 100 mM NaCl, pH 8.3 at RT) were mixed with 3.33 μl of a 10 mM TCEP solution in P5-conjugation buffer. Directly afterwards, 1.67 μl of a 40 mM solution of the linker-payload construct dissolved in DMSO were added. The mixture was shaken at 350 rpm and 25° C. for 16 hours. The reaction mixtures were purified by preparative size-exclusion chromatography with a 25 ml Superdex™ 200 Increase 10/300GL (Cytiva, Sweden) and a flow of 0.8 ml/min eluting with sterile PBS (Merck, Germany). The antibody containing fractions were pooled and concentrated by spin-filtration (Amicon® Ultra-2 mL MWCO: 30 kDa, Merck, Germany).

### In Vitro Cytotoxicity (In Vitro Efficacy)

To investigate direct cytotoxicity of ADCs, respective cells were seeded in a 96-well plate (flat bottom, 5000 cells/well, suspended in 100 μl medium) and incubated for 7 days with increasing concentrations of the ADCs in medium (0-3 μg/ml) to generate a dose-response curve. Before viability analysis, the supernatant over the adherent cells was removed and replaced by fresh medium. Killing was analyzed afterwards, using resazurin (Merck group, Germany) as the cell viability dye at a final concentration of 55 μM. Fluorescence emission at 590 nM was measured on a Microplate reader Infinite M1000 Pro (Tecan). Cell viability was measured by dividing the fluorescence of ADC-treated cells with the fluorescence from control cells, treated in the same way with medium only.

### Serum-Stability of the ADC Constructs

40 μl of normal rat serum, containing the corresponding ADCs in a concentration of 0.4 mg/ml in at least 80% rat serum (Thermo Fisher Scientific, USA) were sterile filtered with UFC30GVOS centrifugal filter units (Merck, Germany) and incubated at 37° C. for 1, 3 and 7 days. Samples for day 0 were directly processed further. The supernatant of 50 μl anti human igG (Fc-Specific) agarose slurry (Sigma Aldrich, United States) was removed by centrifugation and the remaining resin washed three times with 300 μL PBS. The resin was incubated with 40 μl of the serum-ADC mix for 1 h at room temperature. Afterwards, the supernatant was removed and the resin washed 3 times with 300 μL PBS. Following by incubation for 5 minutes with 60 μl 100 mM Glycin buffer pH 2.3 at room temperature. This solution was rebuffed to PBS by using 0.5 mL Zeba™ Spin Desalting Columns with 7K MWCO (Thermo Fisher Scientific, USA). The samples were processed further for MS-measurements, as described above.

### Example 1: Linker-Payload Synthesis

### Dipeptide Synthesis

### L-alanine-4-methylbenzylamide

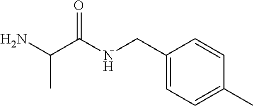

Boc-L-Alanine (270 mg, 1.43 mmol, 1 eq) and Pybop (890 mg, 1.71 mmol, 1.2 eq) were dissolved in DMF and purged with N2 for 5 min. DIPEA (0.75 ml, 4.29 mmol, 3 eq) was added dropwise and 4-methylbenzyamine (208 mg, 1.71 mmol, 1.2 eq) was added. The mixture was stirred at rt overnight. The solvent was removed in vacuo, the residue was dissolved in EtOAc/H₂O and extracted 3 times. The organic phase was washed with brine, dried over MgSO₄ and the solvent was removed. The residue was purified via flash chromatography.

The Boc-protected compound (290 mg, 0.99 mmol) was dissolved in DCM (5 ml) and THF (1.5 ml) was added. Water was added (250 μl) and the mixture was stirred for 15 min. The solvents were removed under N₂ stream. After extraction with saturated NaHCO₃ solution and EtOAc, the organic phase was dried over MgSO₄ and the solvent was removed. (110 mg, 0.57 mmol, 40% over two steps). HR-MS for C₁₁H₁₇N₂O⁺ [M+H]⁺ calcd.:

193.1336, found 193.1335.

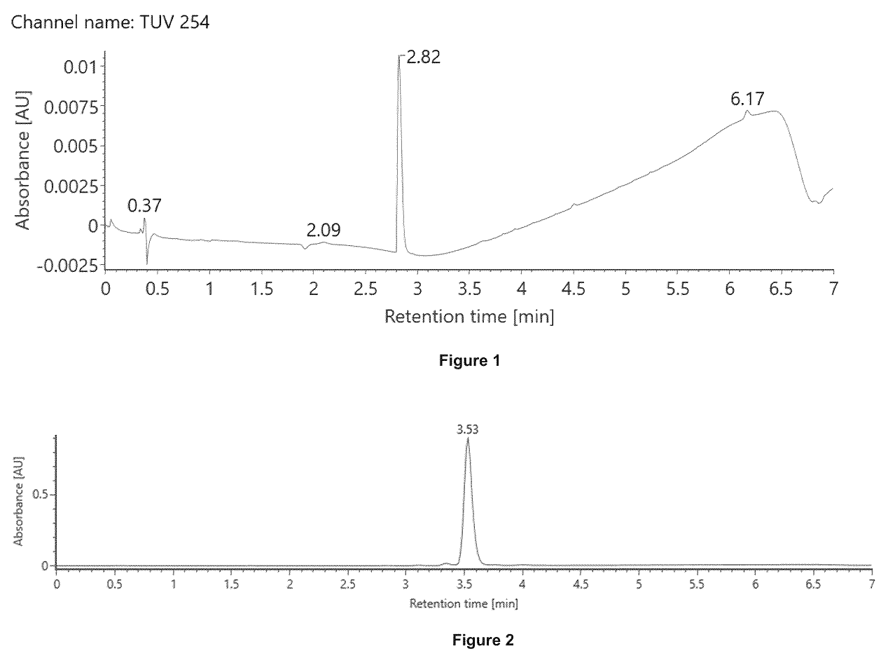

FIG. 1 shows an HPLC/UV chromatogram of the compound L-alanine-4-methylbenzylamide.

### L-alanine-L-alanine-tert.-butylester

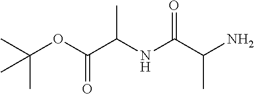

Fmoc-L-Alanine (4 g, 12.84 mmol, 1 eq) was dissolved in DMF. PyBop (8.68 g, 16.72 mmol, 1.3 eq) and DIPEA (8.8 ml, 51.36 mmol, 4 eq) were added and the solution was purged with N₂ for 5 min. L-Ala-OtBu x HCl (2.32 g, 12.84 mmol, 1 eq) was added and the reaction mixture was stirred at rt overnight under argon. The solvent was removed in vacuo, the residue was extracted with H₂O/EtOAc, washed with brine and dried over MgSO₄. After removing the solvent, the crude product was purified via flash chromatography.

The Fmoc-protected compound (3 g, 6.84 mmol) was dissolved in piperidine/DMF (20%) and stirred for 15 min. The solvents were removed in vacuo and the residue was purified via column chromatography (5%-10% MeOH in DCM). (1.26 g, 5.83 mmol, 45% over two steps). HR-MS for C₁₀H₂₁N₂O₃⁺ [M+H]⁺ calcd.: 217.1547, found 217.1616. Analytical Data matched the literature: https://doi.org/10.1021/acsmedchemlett.9b00240)

### β-alanine-L-alanine-tert.-butylester

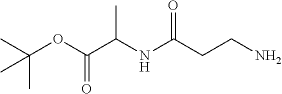

Fmoc-b-Ala-OH (2 g, 6.42 mmol, 1 eq) was dissolved in DMF. PyBop (4.34 g, 8.36 mmol, 1.3 eq) and DIPEA (4.4 ml, 25.68 mmol, 4 eq) were added and the solution was purged with N₂ for 5 min. Ala-OtBu x HCl (1.16 g, 6.42 mmol, 1 eq) was added and the reaction mixture was stirred at rt overnight under argon. The solvent was removed in vacuo, the residue was extracted with H₂O/EtOAc, washed with brine and dried over MgSO₄. After removing the solvent, the crude product was purified via flash chromatography.

The Fmoc-protected compound was dissolved in piperidine/DMF (20%) and stirred for 1 h. The solvents were removed in vacuo and the residue was purified via column chromatography (5%-10% MeOH in DCM). (1.26 g, 5.83 mmol, 45% over two steps) (674 mg, 3.12 mmol, 52% over two steps) HR-MS for C₁₀H₂₁N₂O₃⁺ [M+H]⁺ calcd.: 217.1547, found

217.1572.

### P5(PEG12)-COOH

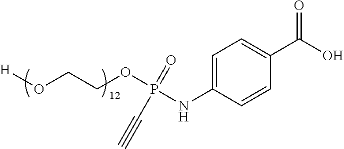

A 25-ml Schlenk flask was charged with 40 mg bis(diisopropylamino)chlorophosphine (150 μmol, 1.00 eq.) under an argon atmosphere, cooled to 0° C. and 330 μL ethynylmagnesium bromide solution (0.5 M in THF, 165 μmol, 1.10 eq.) was added drop wise. The yellowish solution was allowed to warm to room temperature and stirred for further 30 minutes. 245 mg (450 μmol, 3.0 eq.) of the PEG12-Diol, dissolved in 0.8 mL 1H tetrazole solution (0.45 M in MeCN, 360 μmol, 2.50 eq.) were added and the white suspension was stirred overnight at room temperature. The formation of the desired phosphonite was monitored by ³¹P-NMR. 39 mg 4-azidobenzoic-acid (150 μmol, 1.00 eq.), dissolved in 2 mL of DMF was added and the suspension further stirred for 24 h at room temperature. The crude reaction mixture was purified using preparative HPLC (Method D) and lyophilization. (25 mg, 34 μmol, 23%). HR-MS for C₃₃H₅₇NO₁₆P⁺ [M+H]⁺ calcd.: 754.3410, found 754.3398

FIG. 2 shows an HPLC/UV chromatogram of the compound P5(PEG12)-COOH.

### General Method 1 for the Synthesis of Pro5-Phosphoramidates

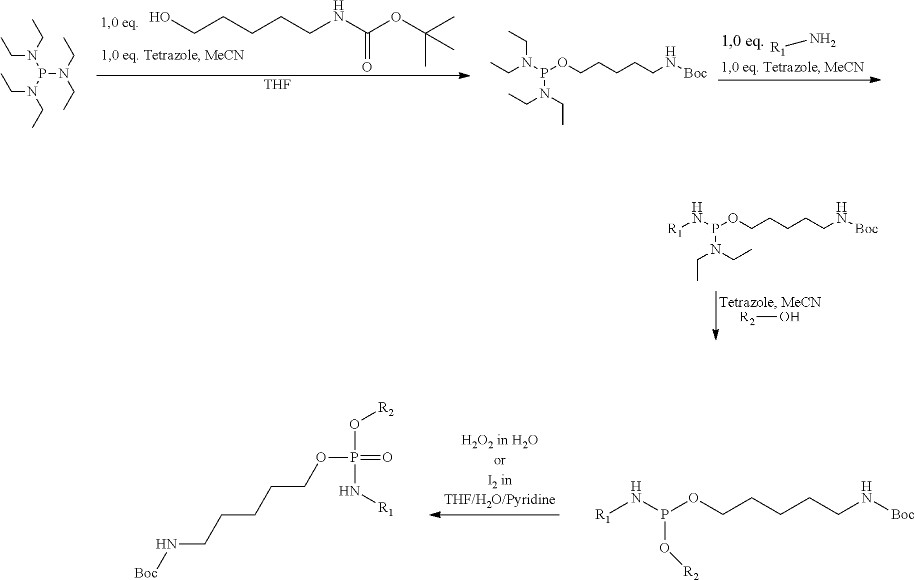

In the above scheme, R¹ is a residue covered by the group

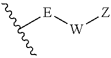

of the compounds and conjugates described herein, and R2 is a drug moiety (D).

A dry 25 ml Schlenk tube with stirring bar was charged with 247 mg (1.0 mmol, 1.0 eq) tris(diethylamino)phosphine in 1 ml THF under a nitrogen atmosphere and cooled to 0° C. with wet ice. Tetrazole solution (0.45 M in acetonitrile, 2.2 ml, 1.0 eq) was added and directly afterwards N-Boc-5-aminopentanol (162 mg, 0.8 mmol, 0.8 eq.) in 1.0 ml THF was added dropwise within 2 min. The cooling bath was removed and the mixture was allowed to stir at rt for 1 h. The reaction mixture was cooled to 0° C. with wet ice. Tetrazole solution (0.45 M in acetonitrile, 2.2 ml, 1.0 eq) was added and directly afterwards an amine, such as L-alanine-tertbutylester (174 mg, 1.2 mmol, 1.2 eq) in 1 ml THF was added dropwise within 2 min. The cooling bath was removed and the mixture was allowed to stir at rt for 1 h. 1.0 mmol of the desired alcohol was pre dried in vacuo for 1 h, dissolved in 2 ml THF and cooled to 0° C. with wet ice. Tetrazole solution (0.45 M in acetonitrile, 2.2 mL, 1.0 eq) and the reaction mixture was added. The mixture was allowed to stir at rt for 1 h. The reaction mixture was oxidized by addition of I₂—Oxidizer (0.15 M solution in THF/pyridine/H₂O=39:10:1) The dropwise addition of the I₂—Oxidizer was continued until the color of the oxidizer remained in the solution. Alternatively, one equivalent of hydrogen peroxide (H₂O₂) as a 30% solution in water was added. The mixture was purified by preparative HPLC.

### O-(5-tert.-butoxy-carbonyl-aminopentyl)-N-(L-alanine-tert.-butylester)-O—(SN38)-Phosphoramidate

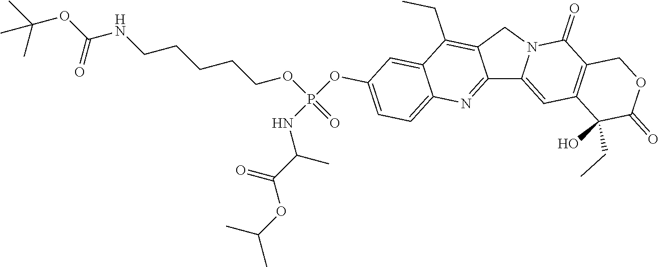 [SMILES: CCc1c2c(nc3ccc(OP(=O)(NC(C)C(=O)OC(C)C)OCCCCCNC(=O)OC(C)(C)C)cc13)-c1cc3c(c(=O)n1C2)COC(=O)[C@]3(O)CC]

The title compound was synthesized in accordance to general Method 1 from 7.3 mg SN-38 (0.0186 mmol). The product was obtained as yellow solid after preparative HPLC (Method C) and lyophilization. (3.07 mg, 0.0039 mmol, 21%). HR-MS for C₃₉H₅₃N₄NaO₁₁P⁺ [M+Na]⁺ calc.: 807.3341 found: 807.3368.

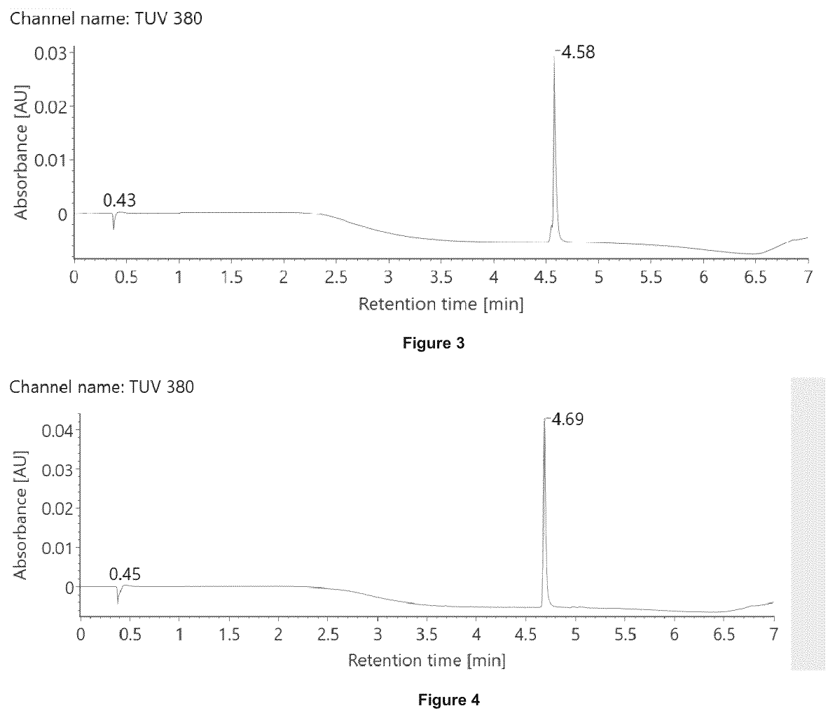

FIG. 3 shows an HPLC/UV chromatogram of the compound O-(5-tert.-butoxy-carbonyl-aminopentyl)-N-(L-alanine-tert.-butylester)-O—(SN38)-Phosphoramidate.

### O-(5-tert.-butoxy-carbonyl-aminopentyl)-N-(4-Methylbenzyl)-O—(SN38)-Phosphoramidate

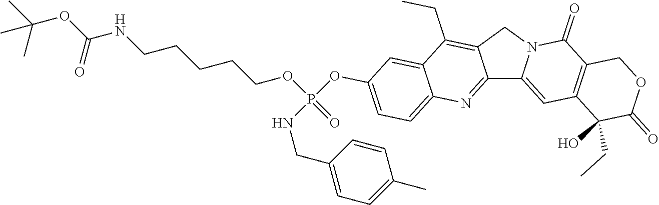 [SMILES: CCc1c2c(nc3ccc(OP(=O)(NCc4ccc(C)cc4)OCCCCCNC(=O)OC(C)(C)C)cc13)-c1cc3c(c(=O)n1C2)COC(=O)[C@]3(O)CC]

The title compound was synthesized in accordance to general Method 1 from 5.2 mg SN-38 (0.0127 mmol). The product was obtained as yellow solid after preparative HPLC (Method C) and lyophilization. (1.1 mg, 0.00145 mmol, 11%). HR-MS for C₄₀H₅₀N₄O₉P⁺ [M+H]⁺ calc.: 761.3310, found 761.3905.

FIG. 4 shows an HPLC/UV chromatogram of the compound O-(5-tert.-butoxy-carbonyl-aminopentyl)-N-(4-Methylbenzyl)-O—(SN38)-Phosphoramidate.

### O-(5-tert.-butoxy-carbonyl-aminopentyl)-N-(L-alanine-4-methylbenzylamide)-O—(SN38)-Phosphoramidate

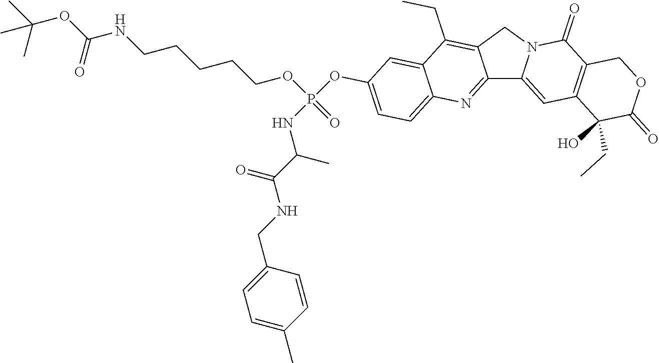 [SMILES: CCc1c2c(nc3ccc(OP(=O)(NC(C)C(=O)NCc4ccc(C)cc4)OCCCCCNC(=O)OC(C)(C)C)cc13)-c1cc3c(c(=O)n1C2)COC(=O)[C@]3(O)CC]

The title compound was synthesized in accordance to general Method 1 from 5.8 mg SN-38 (0.0142 mmol). The product was obtained as yellow solid after preparative HPLC (Method D) and lyophilization. (1.5 mg, 0.0018 mmol, 13%). HR-MS for C₄₃H₅₅N₅O₁₀P⁺ [M+H]⁺ calc.: 832.3681, found 832.3892.

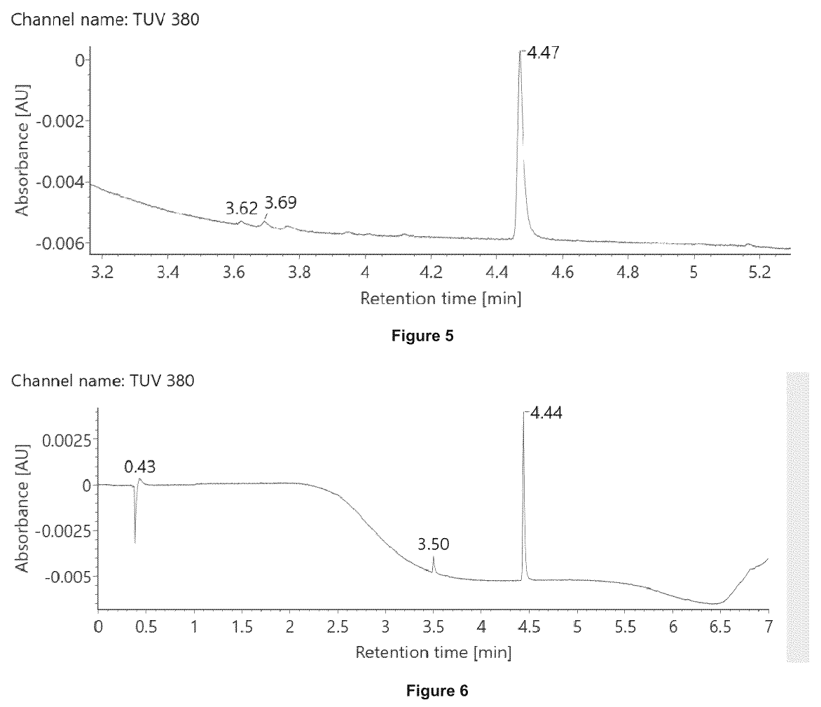

FIG. 5 shows an HPLC/UV chromatogram of the compound O-(5-tert.-butoxy-carbonyl-aminopentyl)-N—(L-alanine-4-methylbenzylamide)-O—(SN38)-Phosphoramidate.

### O-(5-tert.-butoxy-carbonyl-aminopentyl)-N-(L-alanine-L-alanine-tert.-butylester)-O—(SN38)-Phosphoramidate

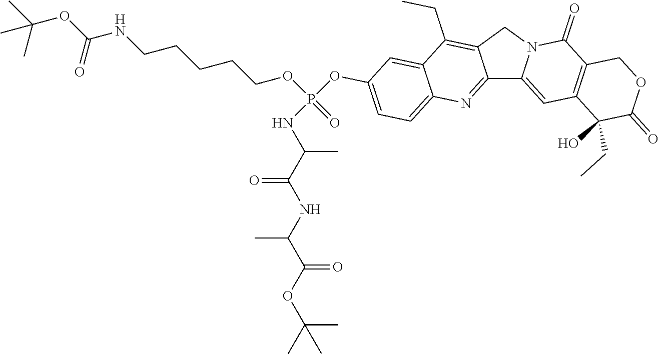 [SMILES: CCc1c2c(nc3ccc(OP(=O)(NC(C)C(=O)NC(C)C(=O)OC(C)(C)C)OCCCCCNC(=O)OC(C)(C)C)cc13)-c1cc3c(c(=O)n1C2)COC(=O)[C@]3(O)CC]

The title compound was synthesized in accordance to general Method 1 from 5.5 mg SN-38 (0.0135 mmol). The product was obtained as yellow solid after preparative HPLC (Method C) and lyophilization. (0.8 mg, 0.000935 mmol, 7%). HR-MS for C₄₂H₅₉N₅O₁₂P⁺ [M+H]⁺ calc.: 856.3892, found 856.4261.

FIG. 6 shows an HPLC/UV chromatogram of the compound O-(5-tert.-butoxy-carbonyl-aminopentyl)-N-(L-alanine-L-alanine-tert.-butylester)-O—(SN38)-Phosphoramidate.

### O-(5-tert.-butoxy-carbonyl-aminopentyl)-N-(β-alanine-L-alanine-tert.-butylester)-O—(SN38)-Phosphoramidate

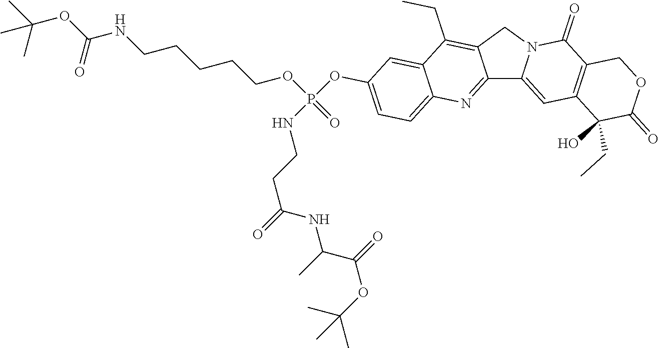 [SMILES: CCc1c2c(nc3ccc(OP(=O)(NCCC(=O)NC(C)C(=O)OC(C)(C)C)OCCCCCNC(=O)OC(C)(C)C)cc13)-c1cc3c(c(=O)n1C2)COC(=O)[C@]3(O)CC]

The title compound was synthesized in accordance to general Method 1 from 8.0 mg SN-38 (0.0196 mmol). The product was obtained as yellow solid after preparative HPLC (Method C) and lyophilization. (4.6 mg, 0.00536 mmol, 27%). HR-MS for C₄₂H₅₉N₅O₁₂P⁺ [M+H]⁺ calcd.: 856.3892, found 856.3880.

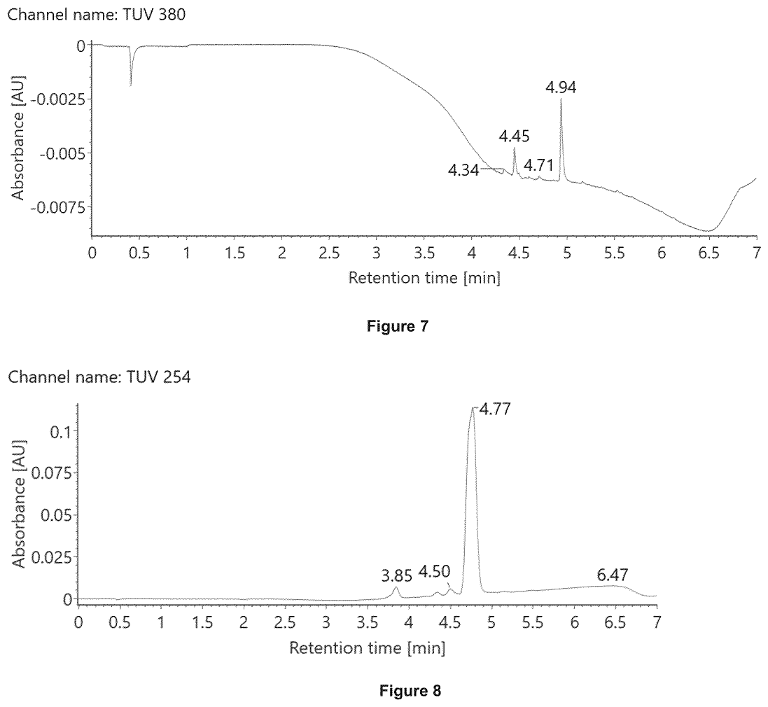

FIG. 7 shows an HPLC/UV chromatogram of the compound O-(5-tert.-butoxy-carbonyl-aminopentyl)-N—(R-alanine-L-alanine-tert.-butylester)-O—(SN38)-Phosphoramidate.

### O-(5-tert.-butoxy-carbonyl-aminopentyl)-N-(L-alanine-tert.-butylester)-O-(DxD)-Phosphoramidate

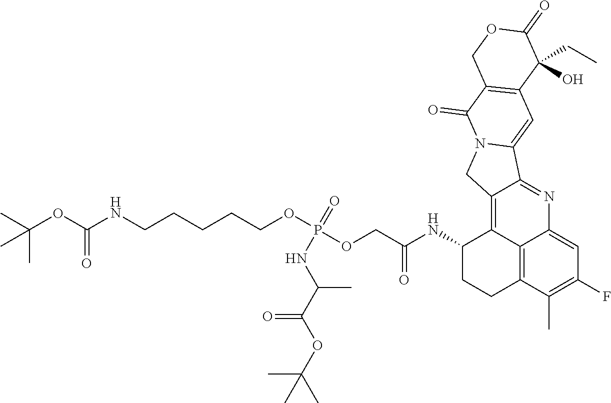 [SMILES: CC[C@@]1(O)C(=O)OCc2c1cc1n(c2=O)Cc2c-1nc1cc(F)c(C)c3c1c2[C@@H](NC(=O)COP(=O)(NC(C)C(=O)OC(C)(C)C)OCCCCCNC(=O)OC(C)(C)C)CC3]

The title compound was synthesized in accordance to general Method 1 from 7.0 mg DxD (0.014 mmol). The product was obtained as yellow solid after preparative HPLC (Method C) and lyophilization. (1.75 mg, 0.0019 mmol, 13.9%). HR-MS for C₄₃H₅₈FN₅O₁₂P⁺ [M+H]⁺ calcd.: 886.3798, found 886.3781.

FIG. 8 shows an HPLC/UV chromatorgram of the compound O-(5-tert.-butoxy-carbonyl-aminopentyl)-N-(L-alanine-tert.-butylester)-O-(DxD)-Phosphoramidate.

### O-(5-tert.-butoxy-carbonyl-aminopentyl)-N-(L-alanine-L-Alanine-tert.-butylester)-O-(DxD)-Phosphoramidate

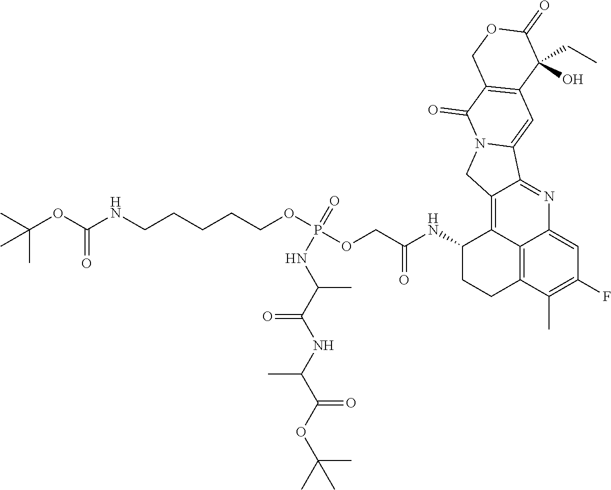 [SMILES: CC[C@@]1(O)C(=O)OCc2c1cc1n(c2=O)Cc2c-1nc1cc(F)c(C)c3c1c2[C@@H](NC(=O)COP(=O)(NC(C)C(=O)NC(C)C(=O)OC(C)(C)C)OCCCCCNC(=O)OC(C)(C)C)CC3]

The title compound was synthesized in accordance to general Method 1 from 10.2 mg DxD (0.021 mmol). The product was obtained as yellow solid after preparative HPLC (Method C) and lyophilization. (5.6 mg, 0.0058 mmol, 27.6%). HR-MS for C₄₆H₆₃FN₆O₁₃P⁺ [M+H]⁺ calcd.: 957.4169, found 957.4149.

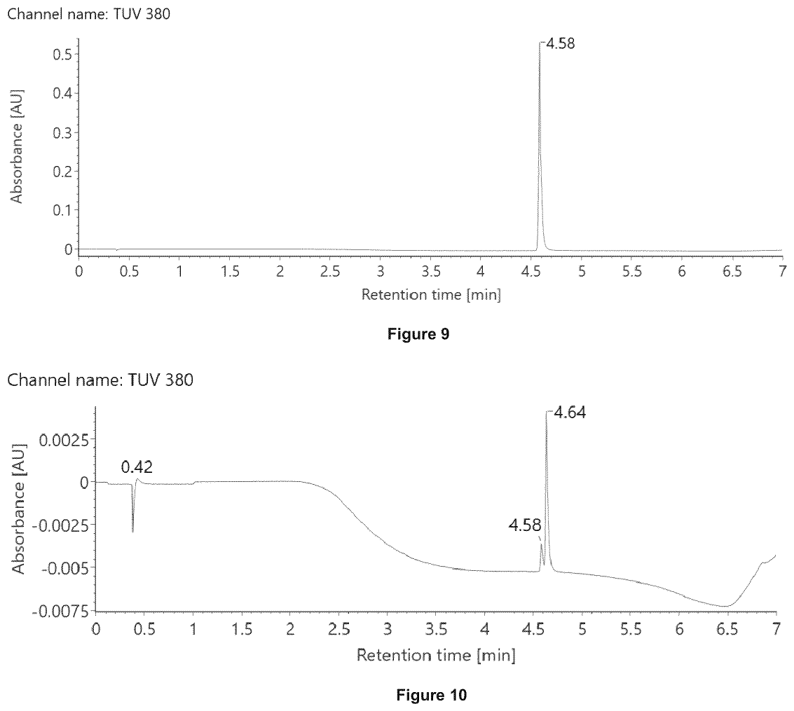

FIG. 9 shows an HPLC/UV chromatogram of the compound O-(5-tert.-butoxy-carbonyl-aminopentyl)-N-(L-alanine-L-Alanine-tert.-butylester)-O-(DxD)-Phosphoramidate.

### Di-O-(5-tert.-butoxy-carbonyl-aminopentyl)-O—(SN38)-Phosphate

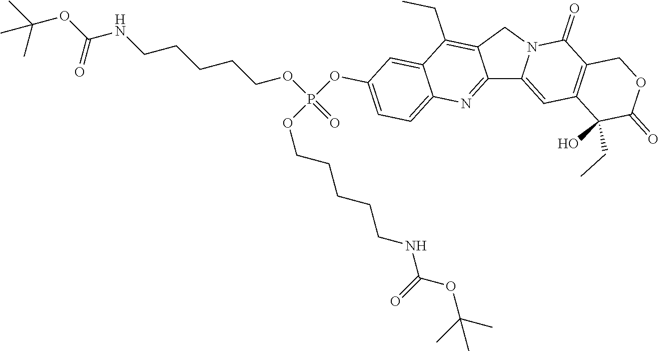 [SMILES: CCc1c2c(nc3ccc(OP(=O)(OCCCCCNC(=O)OC(C)(C)C)OCCCCCNC(=O)OC(C)(C)C)cc13)-c1cc3c(c(=O)n1C2)COC(=O)[C@]3(O)CC]

The title compound was synthesized in accordance to general Method 1 from 7.3 mg SN-38 (0.0186 mmol). The product has been isolated as a side-product from the reaction and was obtained as yellow solid after preparative HPLC (Method C) and lyophilization. (3.76 mg, 0.0045 mmol, 24%). HR-MS for C₄₂H₆₀N₄O₁₂P⁺ [M+H]⁺ calcd.:

843.3940, found 843.4021.

FIG. 10 shows an HPLC/UV chromatogram of the compound Di-O-(5-tert.-butoxy-carbonyl-aminopentyl)-O—(SN38)-Phosphate.

### General Method 2 for the Boc Deprotection in the Presence of the Tert-Butyl Ester, Followed by Amide Coupling to the Deprotected Amine

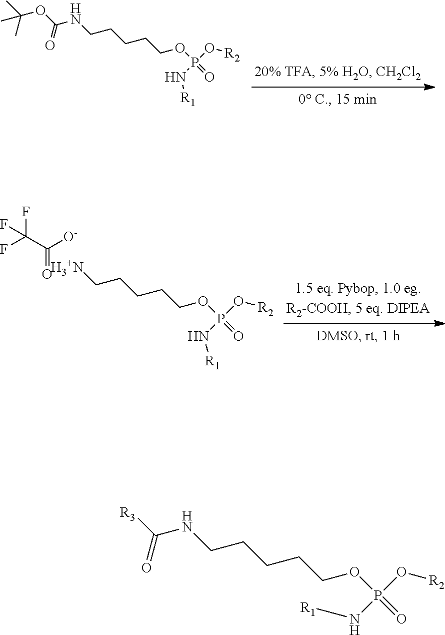

In the above scheme, R1 is a residue covered by the group

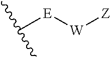

of the compounds and conjugates described herein, and R2 is a drug moiety (D), R3 is part of L*.

The boc protected phosphoramidates were dissolved in 0.5 ml DCM and 5% H₂O were added. After cooling the mixture to 0° C. in an ice bath, 0.1 ml TFA (20%) were added and the solution was stirred at 0° C. for 15 min. The solvents were removed under N2 stream and the product was dried in vacuo. The products were directly transferred to the next step without any further purification.

The residue was dissolved in 0.5 ml DMSO. 1.5 eq. PyBop, dissolved in DMSO and 5.0 eq. DIPEA were added. After 5 minutes, 1.0 eq of desired carboxylic acid dissolved in DMSO was added and the mixture was stirred at RT for 1h. The mixture was diluted with H2O/MeCN (2:1) and directly subjected to preparative HPLC purification.

### O—(P5(OEt)-amidopentyl)-N-(L-Alanine-tert.-butylester)-O—(SN38)-Phosphoramidate

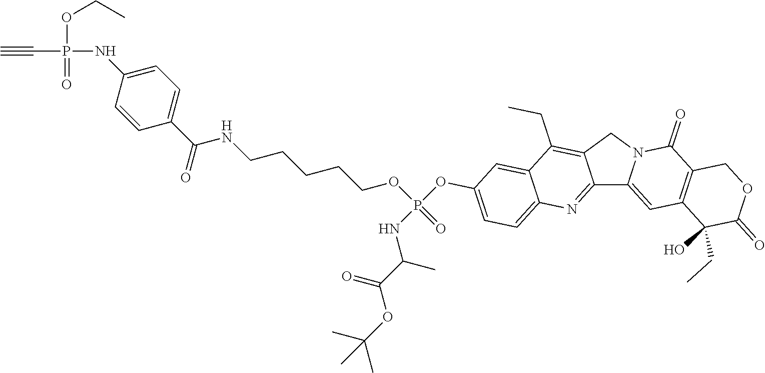 [SMILES: C#CP(=O)(Nc1ccc(C(=O)NCCCCCOP(=O)(NC(C)C(=O)OC(C)(C)C)Oc2ccc3nc4c(c(CC)c3c2)Cn2c-4cc3c(c2=O)COC(=O)[C@]3(O)CC)cc1)OCC]

The title compound was synthesized in accordance to general Method 2 from 0.9 mg O-(5-tert.-butoxy-carbonyl-aminopentyl)-N-(L-alanine-tert.-butylester)-O—(SN38)-Phosphoramidate (0.00114 mmol, 1 eq) and 5.9 μl of P5(OEt)-COOH (0.2M stock in DMSO, 0.00114 mmol, 1 eq). The product was obtained as yellow solid after preparative HPLC (Method C) and lyophilization. (0.4 mg, 0.000435 mmol, 38% over two steps) HR-MS for C₄₅H₅₆N₅O₁₂P₂⁺ [M+H]⁺ calcd.: 920.3395, found: 920.3428.

FIG. 11 shows an HPLC/UV chromatogram of the compound O—(P5(OEt)-amidopentyl)-N-(L-Alanine-tert.-butylester)-O—(SN38)-Phosphoramidate.

### O—(P5(PEG2)-amidopentyl)-N-(L-Alanine-tert.-butylester)-O—(SN38)-Phosphoramidate

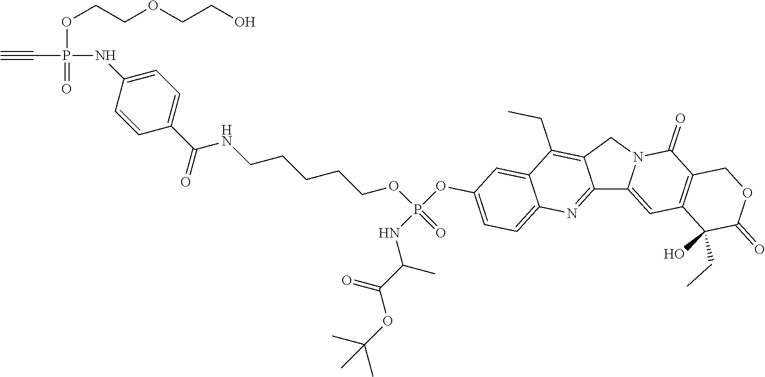 [SMILES: C#CP(=O)(Nc1ccc(C(=O)NCCCCCOP(=O)(NC(C)C(=O)OC(C)(C)C)Oc2ccc3nc4c(c(CC)c3c2)Cn2c-4cc3c(c2=O)COC(=O)[C@]3(O)CC)cc1)OCCOCCO]

The title compound was synthesized in accordance to general Method 2 from 16.15 mg O-(5-tert.-butoxy-carbonyl-aminopentyl)-N-(L-alanine-tert.-butylester)-O—(SN38)-Phosphoramidate (0.02058 mmol, 1 eq) and 103 μl of P5(PEG2)-COOH (doi.org/10.1002/anie.201904193) 0.2M stock in DMSO, 0.02058 mmol, 1 eq). The product was obtained as yellow solid after preparative HPLC (Method C) and lyophilization. (9.2 mg, 0.00939 mmol, 53% over two steps) HR-MS for C₄₇H₆₀N₅O₁₄P₂⁺ [M+H]⁺ calcd.: 980.3607, found 980.3638.

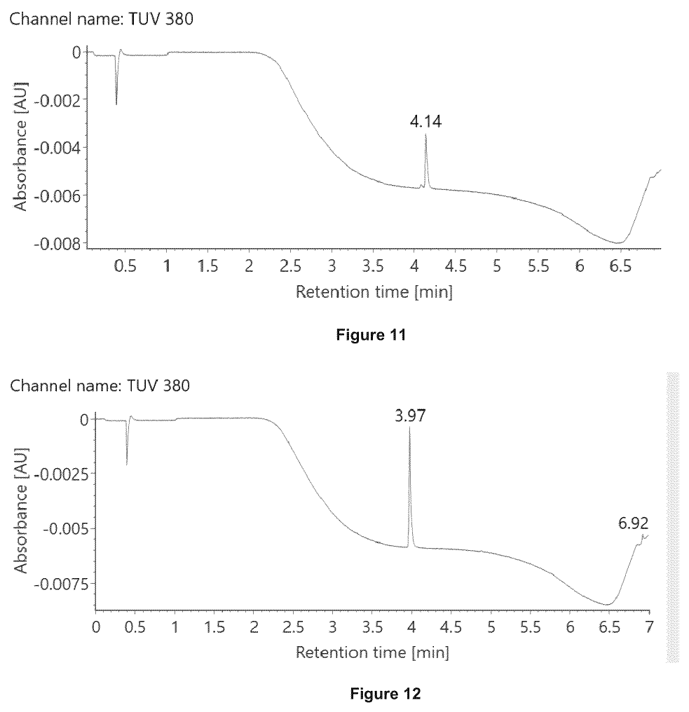

FIG. 12 shows an HPLC chromatogram of the compound O—(P5(PEG2)-amidopentyl)-N-(L-Alanine-tert.-butylester)-O—(SN38)-Phosphoramidate.

### O—(P5(PEG12)-amidopentyl)-N-(L-Alanine-tert.-butylester)-O—(SN38)-Phosphoramidate

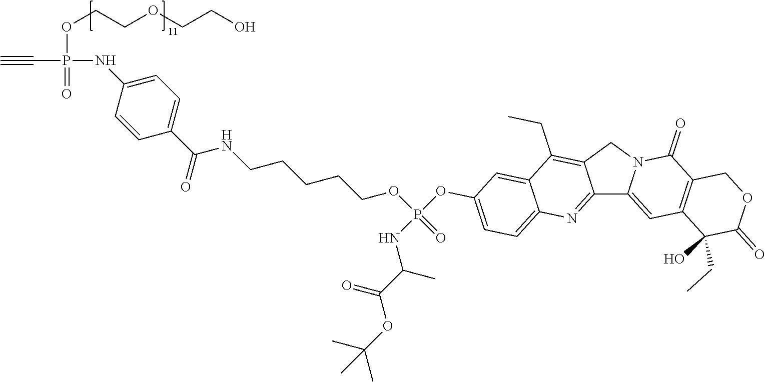

The title compound was synthesized in accordance to general Method 2 from 3.8 mg mg O-(5-tert.-butoxy-carbonyl-aminopentyl)-N-(L-alanine-tert.-butylester)-O—(SN38)-Phosphoramidate (0.0045 mmol, 1 eq) and 23 μl of P5(PEG12)-COOH (0.2M stock in DMSO, 0.0067 mmol, 1 eq). The product was obtained as yellow solid after preparative HPLC (Method C) and lyophilization. (1.05 mg, 0.00076 mmol, 17% over two steps). HR-MS for C₆₇H₁₀₁NO₂₄P₂²⁺ [M+H]²⁺ calcd.: 710.8151, found 710.8147.

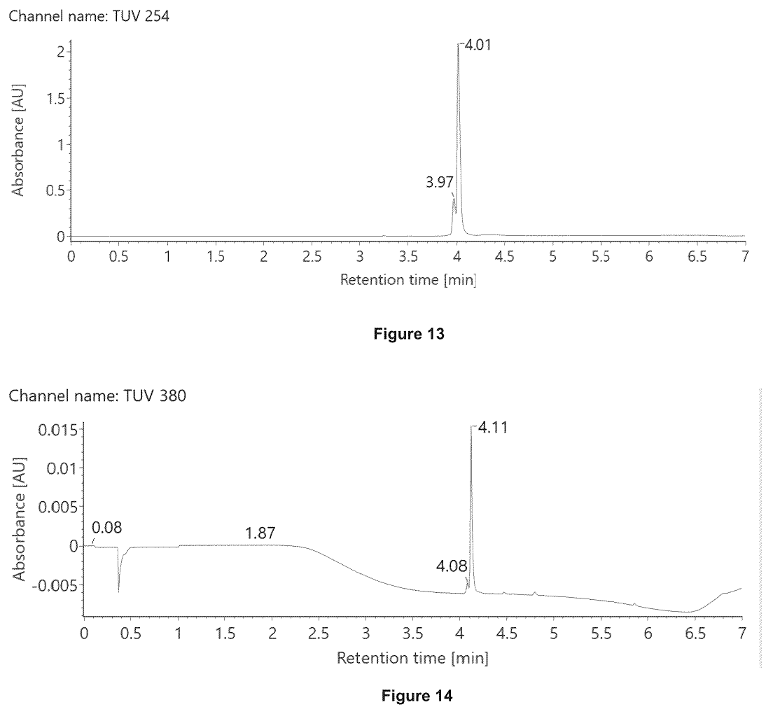

FIG. 13 shows an HPLC/UV chromatogram of the compound O—(P5(PEG12)-amidopentyl)-N-(L-Alanine-tert.-butylester)-O—(SN38)-Phosphoramidate.

### O—(P5(PEG12)-amidopentyl)-N-(4-Methylbenzvl)-O—(SN38)-Phosphoramidate

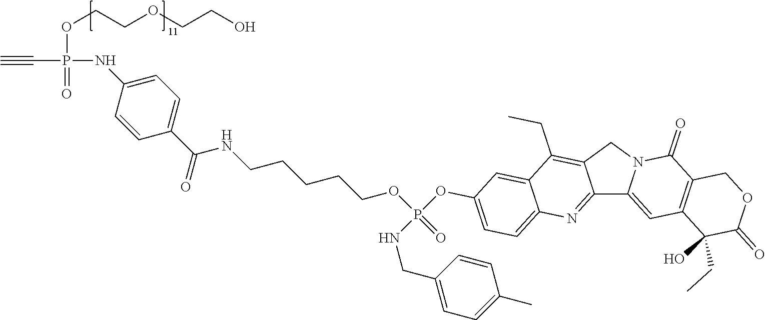

The title compound was synthesized in accordance to general Method 2 from 1.1 mg O-(5-tert-butoxy-carbonyl-aminopentyl)-N-(4-Methylbenzyl)-O—(SN38)-Phosphoramidate (0.00145 mmol, 1 eq) and 7.3 μl of P5(PEG12)-COOH (0.2M stock in DMSO, 0.00145 mmol, 1 eq). The product was obtained as yellow solid after preparative HPLC (Method C) and lyophilization. (0.98 mg, 0.000702 mmol, 48% over two steps). HR-MS for C₆₈H₉₇N₅O₂₂P₂²⁺ [M+2H]²⁺ calcd.: 698.8045, found 698.8568.

FIG. 14 shows an HPLC/UV chromatogram of the compound O—(P5(PEG12)-amidopentyl)-N-(4-Methylbenzyl)-O—(SN38)-Phosphoramidate.

### O—(P5(PEG12)-amidopentyl)-N-(L-alanine-L-Alanine-tert.-butylester)-O—(SN38)-Phosphoramidate

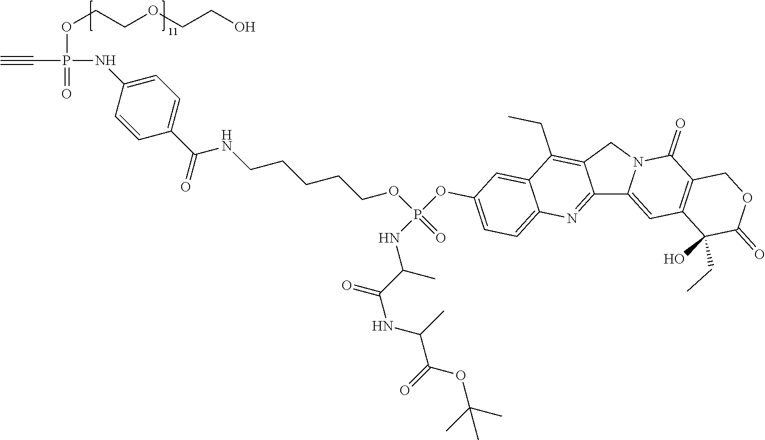

The title compound was synthesized in accordance to general Method 2 from 0.8 mg O-(5-tert.-butoxy-carbonyl-aminopentyl)-N-(L-alanine-L-Alanine-tert.-butylester)-O—(SN38)-Phosphoramidate (0.000935 mmol, 1 eq) and 4.7 μl of P5(PEG12)-COOH (0.2M stock in DMSO, 0.000935 mmol, 1 eq). The product was obtained as yellow solid after preparative HPLC (Method C) and lyophilization. (0.25 mg, 0.000168 mmol, 18% over two steps). HR-MS for C₇₀H₁₀₆N₆O₂₅P₂⁺ [M+2H]²⁺ calcd.: 746.3336, found 746.3315.

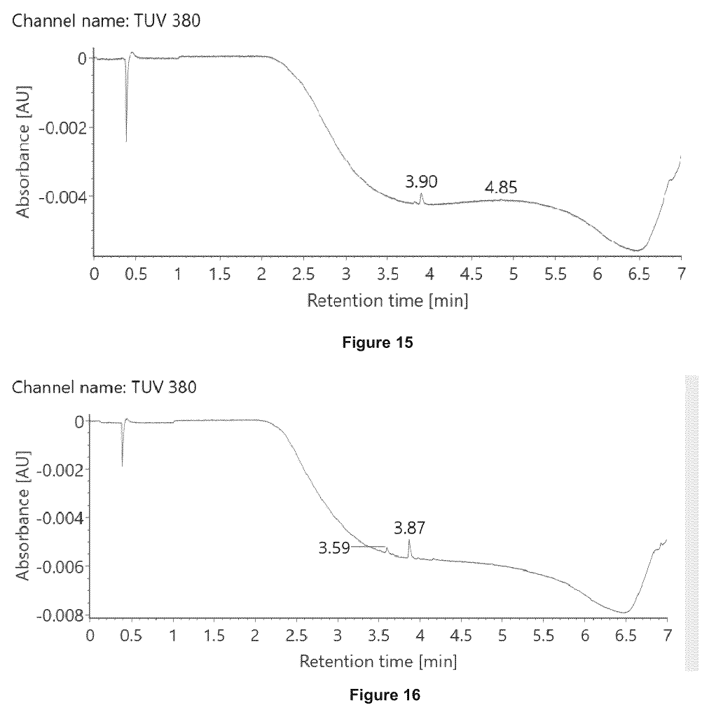

FIG. 15 shows an HPLC chromatogram of the compound O—(P5(PEG12)-amidopentyl)-N-(L-alanine-L-Alanine-tert.-butylester)-O—(SN38)-Phosphoramidate

### O—(P5(PEG12)-amidopentyl)-N-(β-alanine-L-alanine-tert.-butylester)-O—(SN38)

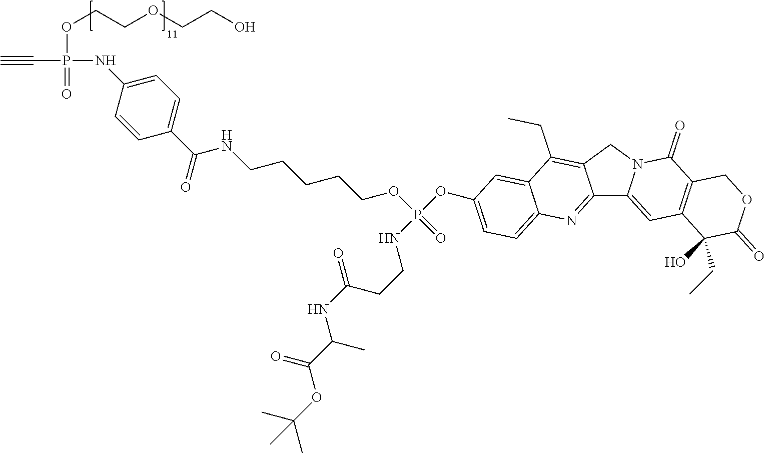

The title compound was synthesized in accordance to general Method 2 from 2.3 mg O-(5-tert.-butoxy-carbonyl-aminopentyl)-N-(β-alanine-L-alanine-tert.-butylester)-O—(SN38)-Phosphoramidate (0.0026 mmol, 1 eq) and 13 μl of P5(PEG12)-COOH (0.2M stock in DMSO, 0.0026 mmol, 1 eq). The product was obtained as yellow solid after preparative HPLC (Method C) and lyophilization. (1.56 mg, 0.00109 mmol, 41% over two steps). HR-MS for C₇₀H₁₀₆N₆O₂₅P₂⁺ [M+2H]²⁺ calcd.: 746.3336, found: 746.3318.

FIG. 16 shows an HPLC/UV chromatogram of the compound O—(P5(PEG12)-amidopentyl)-N-(β-alanine-L-alanine-tert.-butylester)-O—(SN38)-Phosphoramidate.

### O—P5(PEG12)-amidopentyl-Phosphate-O-(5-Aminopentyl)-O-SN38-Phosphate TFA salt

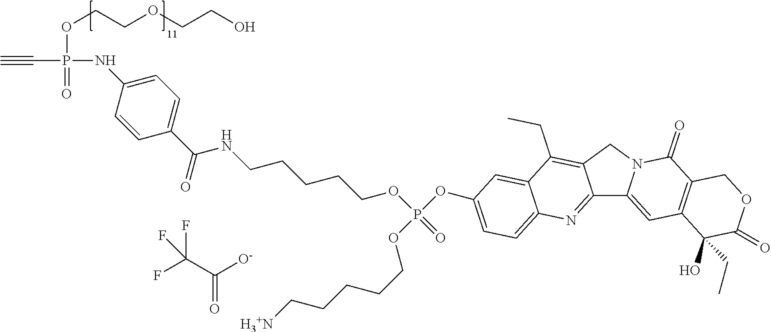

The title compound was synthesized in accordance to general Method 2 from 3.8 mg Di-O-(5-tert.-butoxy-carbonyl-aminopentyl)-O—(SN38)-Phosphate (0.0045 mmol, 1 eq) and 23 μl of P5(PEG12)-COOH (0.2M stock in DMSO, 0.0045 mmol, 1 eq). The product was obtained as yellow solid after preparative HPLC (Method C) and lyophilization. (1.05 mg, 0.00076 mmol, 17% over two steps). HR-MS for C₆₅H₉₉N₅O₂₃P₂⁺ [M+2H]²⁺ calcd.: 689.8098, found 689.8564.

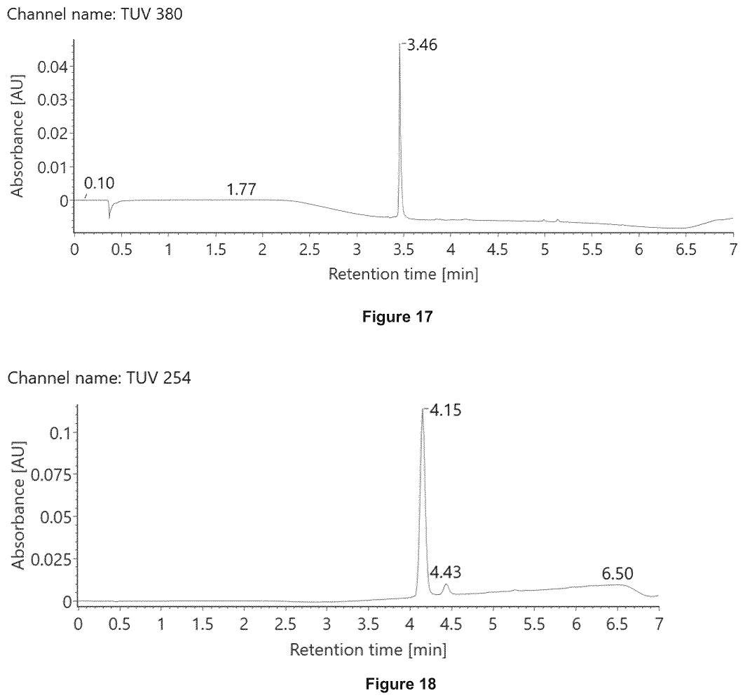

FIG. 17 shows an HPLC/UV chromatogram of the compound O—P5(PEG12)-amidopentyl-Phosphate-O-(5-Aminopentyl)-O-SN38-Phosphate TFA salt.

### O—(P5(PEG2)-amidopentyl)-N-(L-alanine-L-alanine-tert.-butylester)-O-(DxD)-Phosphoramidate

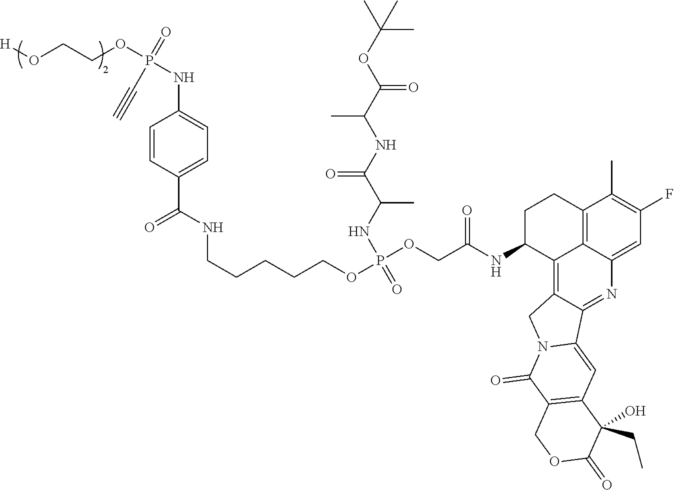

The title compound was synthesized in accordance to general Method 2 from 2.9 mg of O-(5-tert.-butoxy-carbonyl-aminopentyl)-N-(L-alanine-L-alanine-tert.-butylester)-O—(DxD)-Phosphoramidate (0.003 mmol, 1 eq). The product was obtained as yellow solid after preparative HPLC (Method C) and lyophilization. (1 mg, 0.87 μmol, 30% over two steps). HR-MS for C₅₄H₆₉FN₇O₁₆P₂⁺ [M+H]⁺ calcd.: 1152.4255, found 1152.4267.

FIG. 18 shows an HPLC/UV chromatogram of the compound O—(P5(PEG2)-amidopentyl)-N-(L-alanine-L-alanine-tert.-butylester)-O-(DxD)-Phosphoramidate.

### O—(P5(PEG2)-amidopentyl)-N-(L-alanine-tert.-butylester)-O-(DxD)-Phosphoramidate

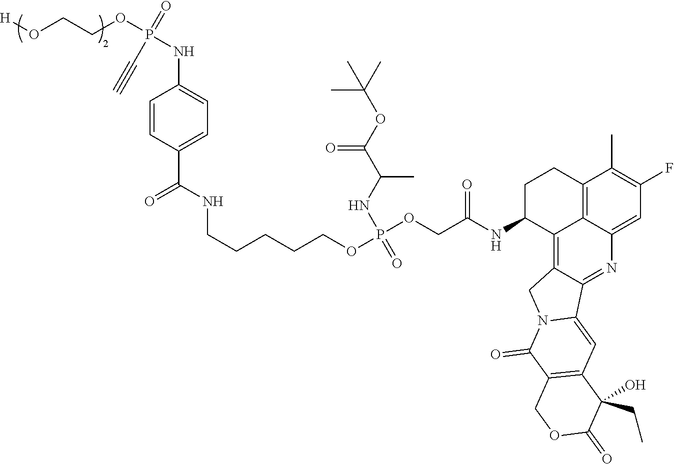

The title compound was synthesized in accordance to general Method 2 from 1.6 mg of O-(5-tert.-butoxy-carbonyl-aminopentyl)-N-(L-alanine-tert.-butylester)-O-(DxD)-Phosphoramidate (0.002 mmol, 1 eq). The product was obtained as yellow solid after preparative HPLC (Method C) and lyophilization. (1.7 mg, 0.0016 mmol, 83% over two steps). HR-MS for C₅₁H₆₄FN₆O₁₅P₂⁺ [M+H]⁺ calcd.: 1081.3883, found 1081.3879.

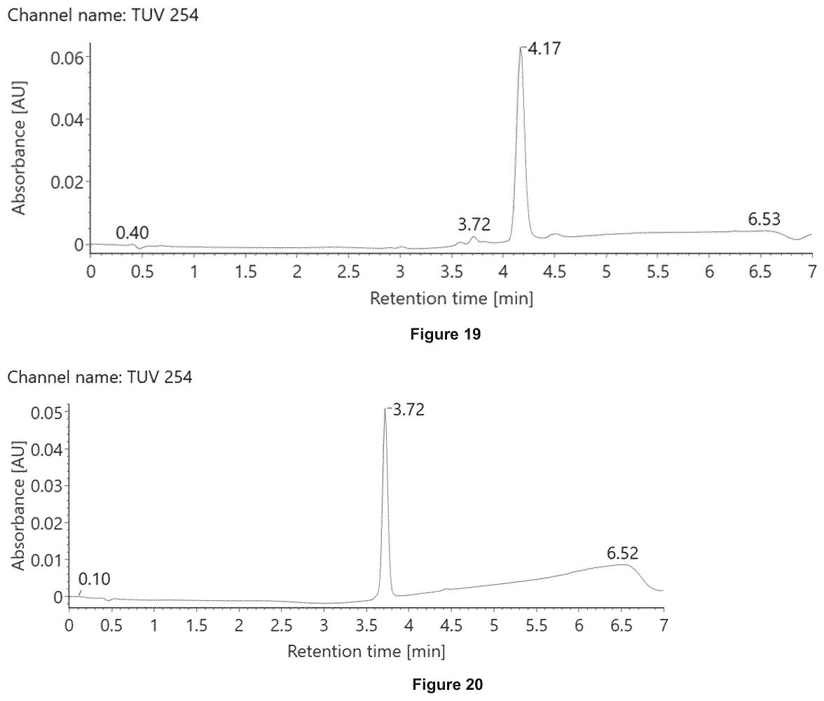

FIG. 19 shows an HPLC/UV chromatogram of the compound O—(P5(PEG2)-amidopentyl)-N-(L-alanine-tert.-butylester)-O-(DxD)-Phosphoramidate.

### O—(P5(PEG2)-amidopentyl)-N-(L-alanine-L-alanine-COOH)—O-(DxD)-Phosphoramidate

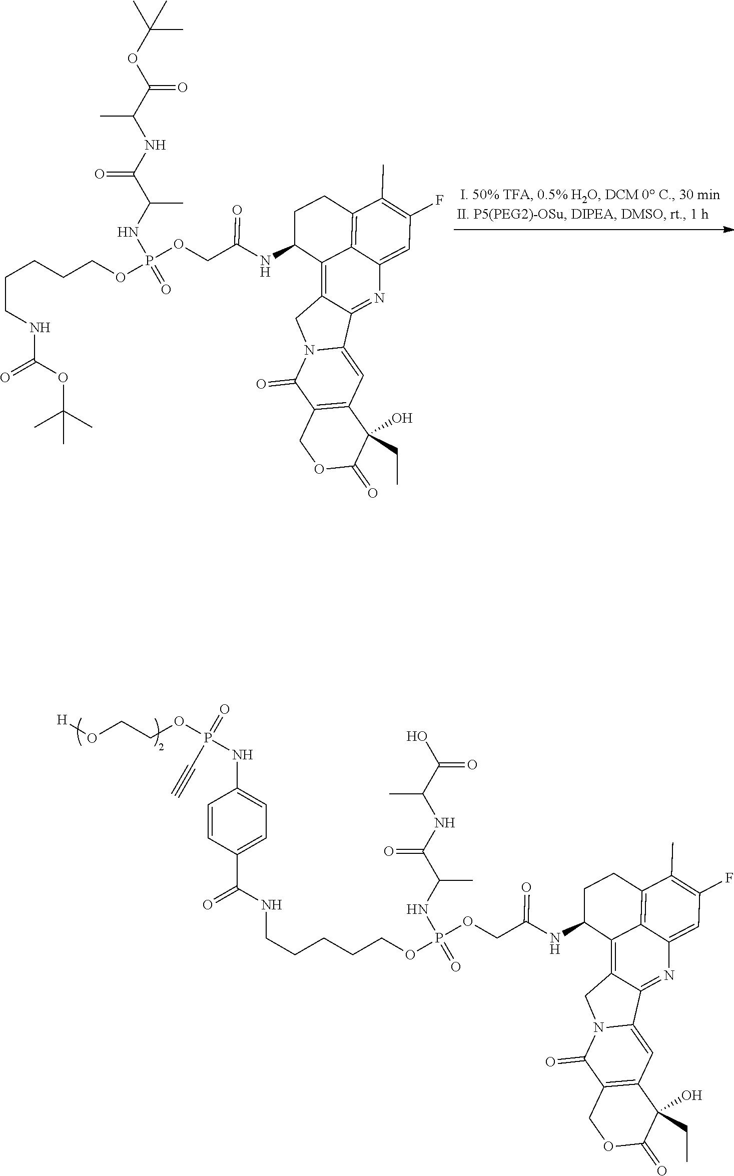

2.9 mg of O-(5-tert.-butoxy-carbonyl-aminopentyl)-N-(L-alanine-L-alanine-tert.-butylester)-O-(DxD)-Phosphoramidate (0.003 mmol) were dissolved in 0.5 mL of 50% TFA in DCM containing 5% water and cooled with wet ice. The reaction was stirred for 45 minutes on ice or until the Boc- and tBu-protecting groups were completely removed (followed by HPLC-MS). The solvents were removed in nitrogen stream and the residue was dried under high vacuum at a Schlenk-line. The dried deprotected Alco5 building block was dissolved in 60 μL DMSO and incubated with 30 μL of a 200 mM P5-PEG2-OSu solution (0.006 mmol, 2 eq) and 5 μL DIPEA (0.030 mmol, 10 eq). The reaction was stirred for 1 h at room temperature. Subsequently, the reaction was diluted with H2O/MeCN (1:1) and purified by HPLC (40 to 99% MeCN in H2O+0.1% TFA). 0.6 mg of the title compound were isolated (0.55 μmol, 18.4%). HR-MS for C₅₀H₆₁FN₇O₁₆P₂⁺ [M+H]⁺ calcd.: 1096.3629, found

1096.36135.

FIG. 20 shows an HPLC/UV chromatogram of the compound O—(P5(PEG2)-amidopentyl)-N-(L-alanine-L-alanine-COOH)—O-(DxD)-Phosphoramidate.

### γ-Aminobutyric acid-L-alanine-tert.-butylester

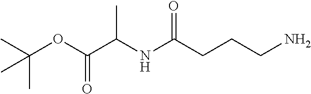 [SMILES: CC(NC(=O)CCCN)C(=O)OC(C)(C)C]

Fmoc γ-Aminobutyric acid (155 mg, 0.5 mmol, 1 eq) was dissolved in DMF. PyBop (321 mg, 0.6 mmol, 1.3 eq) and DIPEA (0.33 ml, 1.9 mmol, 4 eq) were added and the solution was purged with N₂ for 5 min. Ala-OtBu x HCl (86 mg, 0.5 mmol, 1 eq) was added and the reaction mixture was stirred at rt for 1 h under argon. The solution was diluted with EtOAc and washed with sat. NaHCO₃. The aqueous layer was extracted with EtOAc and the combined organic layers were washed with brine and dried over MgSO₄. After removing the solvent, the crude product was purified via flash chromatography.

The Fmoc-protected compound was dissolved in piperidine/DMF (20%) and stirred for 30 min. The solvents were removed in vacuo and the residue was purified via column chromatography (5%-10% MeOH in DCM). (57 mg, 0.25 mmol, 45% over two steps) MS for C₁₁H₂₃N₂O₃⁺ [M+H]⁺ calcd.: 231.17, found 231.14.

### 5-Aminovaleric acid-L-alanine-tert.-butylester

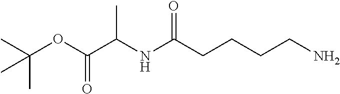 [SMILES: CC(NC(=O)CCCCN)C(=O)OC(C)(C)C]

Fmoc 5-Aminovaleric acid (170 mg, 0.5 mmol, 1 eq) was dissolved in DMF. PyBop (338 mg, 0.7 mmol, 1.3 eq) and DIPEA (0.35 ml, 2 mmol, 4 eq) were added and the solution was purged with N₂ for 5 min. Ala-OtBu x HCl (91 mg, 0.5 mmol, 1 eq) was added and the reaction mixture was stirred at rt for 1 h under argon. The solution was diluted with EtOAc and washed with sat. NaHCO₃. The aqueous layer was extracted with EtOAc and the combined organic layers were washed with brine and dried over MgSO₄. After removing the solvent, the crude product was purified via flash chromatography.

The Fmoc-protected compound was dissolved in piperidine/DMF (20%) and stirred for 30 min. The solvents were removed in vacuo and the residue was purified via column chromatography (5%-10% MeOH in DCM). (114 mg, 0.47 mmol, 94% over two steps) MS for C₁₂H₂₅N₂O₃⁺ [M+H]⁺ calcd.: 245.2, found 245.1.

### O-(5-tert.-butoxy-carbonyl-aminopentyl)-N—(B3-alanine-L-Alanine-tert.-butylester)-O-(DxD)-Phosphoramidate

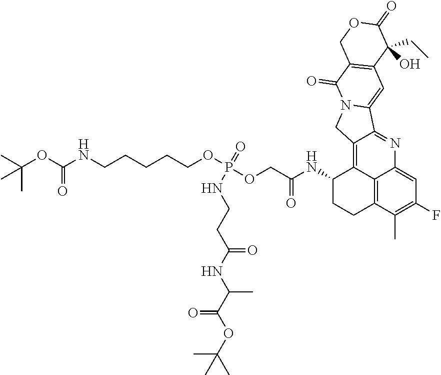 [SMILES: CC[C@@]1(O)C(=O)OCc2c1cc1n(c2=O)Cc2c-1nc1cc(F)c(C)c3c1c2[C@@H](NC(=O)COP(=O)(NCCC(=O)NC(C)C(=O)OC(C)(C)C)OCCCCCNC(=O)OC(C)(C)C)CC3]

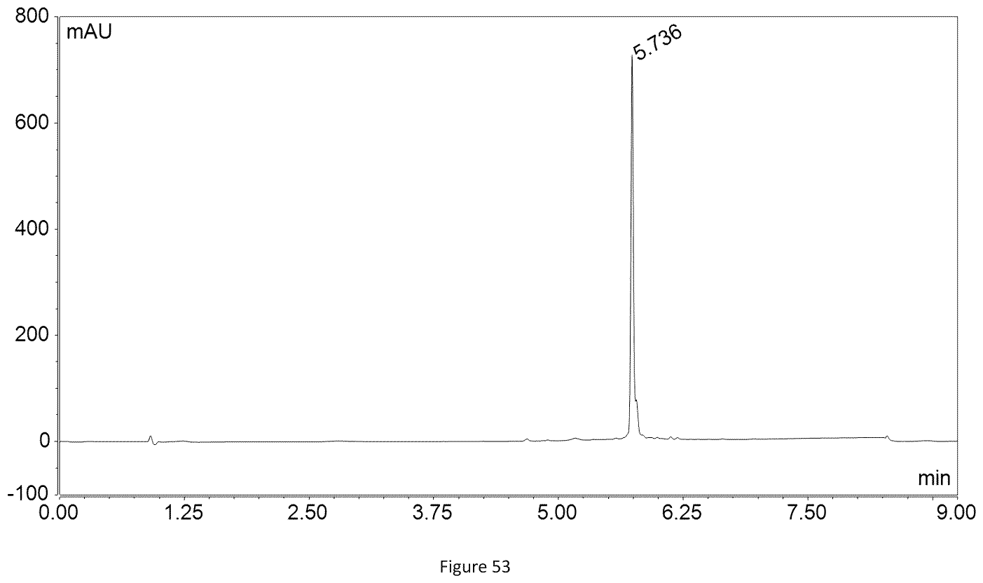

The title compound was synthesized in accordance to general Method 1 from 5.8 mg DxD (0.012 mmol). The product was obtained as brown solid after preparative HPLC (Method C) and lyophilization. (1.05 mg, 0.0011 mmol, 9.1%). HR-MS for C₄₆H₆₃FN₆O₁₃P⁺ [M+H]⁺ calcd.: 957.4169, found 957.4166 FIG. 53 shows an HPLC/UV chromatogram of the compound O-(5-tert.-butoxy-carbonyl-aminopentyl)-N—(β-alanine-L-Alanine-tert.-butylester)-O-(DxD)-Phosphoramidate.

### O-(5-tert.-butoxy-carbonyl-aminopentyl)-N-(γ-Aminobutyric acid-L-Alanine-tert.-butylester)-O- (DxD)-Phosphoramidate

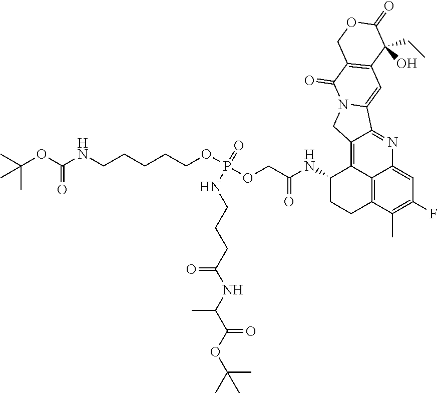 [SMILES: CC[C@@]1(O)C(=O)OCc2c1cc1n(c2=O)Cc2c-1nc1cc(F)c(C)c3c1c2[C@@H](NC(=O)COP(=O)(NCCCC(=O)NC(C)C(=O)OC(C)(C)C)OCCCCCNC(=O)OC(C)(C)C)CC3]

The title compound was synthesized in accordance to general Method 1 from 5.8 mg DxD (0.012 mmol). The product was obtained as yellow solid after preparative HPLC (Method C) and lyophilization. (3.3 mg, 0.0034 mmol, 28%). HR-MS for C₄₇H₆₅FN₆O₁₃P⁺ [M+H]⁺ calcd.: 971.4326, found 971.4333.

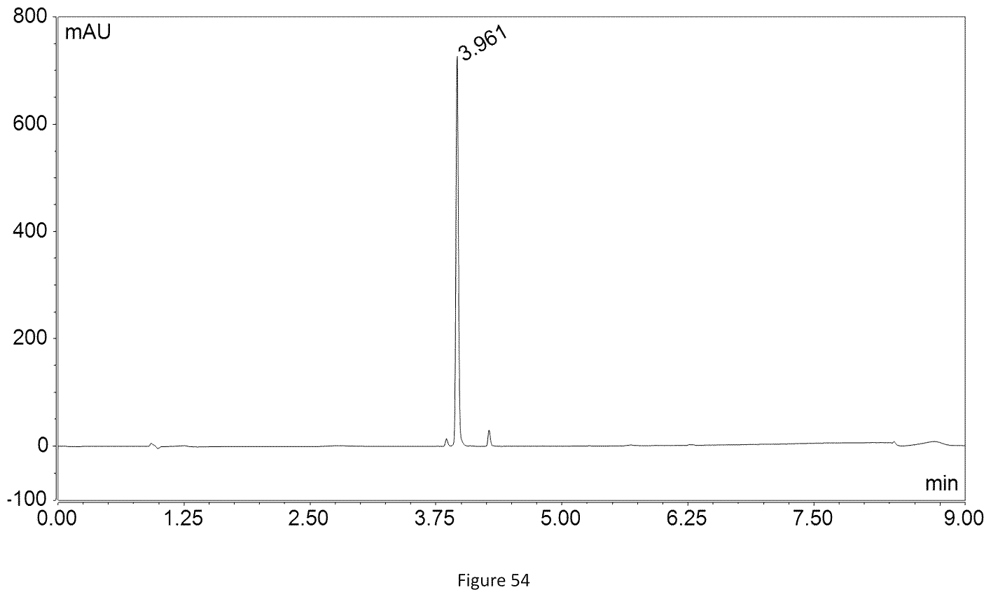

FIG. 54 shows an HPLC/UV chromatogram of the compound O-(5-tert.-butoxy-carbonyl-aminopentyl)-N-(γ-Aminobutyric acid-L-Alanine-tert.-butylester)-O-(DxD)-Phosphoramidate.

### O-(5-tert.-butoxy-carbonyl-aminopentyl)-N-(5-Aminovaleric acid-L-Alanine-tert.-butylester)-O-(DxD)-Phosphoramidate

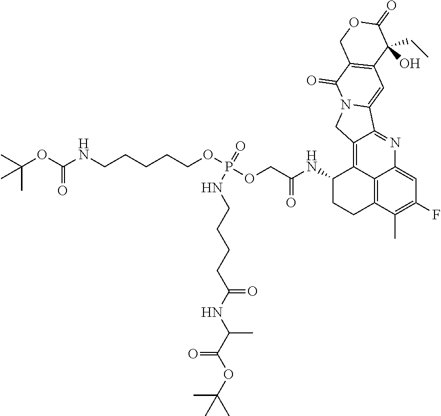 [SMILES: CC[C@@]1(O)C(=O)OCc2c1cc1n(c2=O)Cc2c-1nc1cc(F)c(C)c3c1c2[C@@H](NC(=O)COP(=O)(NCCCCC(=O)NC(C)C(=O)OC(C)(C)C)OCCCCCNC(=O)OC(C)(C)C)CC3]

The title compound was synthesized in accordance to general Method 1 from 5.7 mg DxD (0.012 mmol). The product was obtained as yellow solid after preparative HPLC (Method C) and lyophilization. (4.6 mg, 0.0047 mmol, 39%). HR-MS for C₄₆H₆₃FN₆O₁₃P⁺ [M+H]⁺ calcd.: 985.4482, found 985.4472.

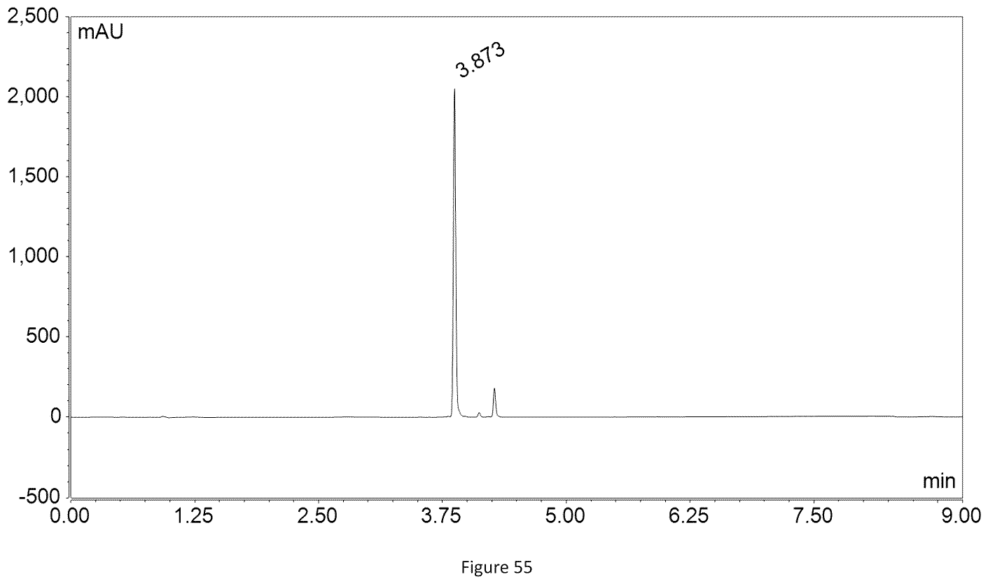

FIG. 55 shows an HPLC/UV chromatogram of the compound O-(5-tert.-butoxy-carbonyl-aminopentyl)-N-(5-Aminovaleric acid-L-Alanine-tert.-butylester)-O-(DxD)-Phosphoramidate.

### O-(5-tert.-butoxy-carbonyl-aminopentyl)-N-(L-alanine-tert.-butylester)-O—(OTS-964)-Phosphoramidate

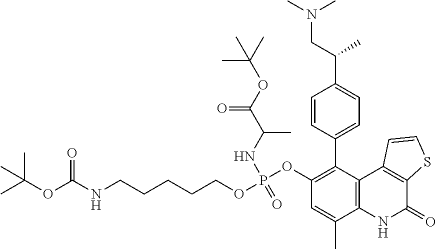 [SMILES: Cc1cc(OP(=O)(NC(C)C(=O)OC(C)(C)C)OCCCCCNC(=O)OC(C)(C)C)c(-c2ccc([C@@H](C)CN(C)C)cc2)c2c1[nH]c(=O)c1sccc12]

The title compound was synthesized in accordance to general Method 1 from 6 mg OTS-964 (0.014 mmol). The product was obtained as solid after preparative HPLC (Method C) and lyophilization. (8.8 mg, 0.0112 mmol, 80.1%). MS for C₄₀H₅₈N₄O₈PS⁺ [M+H]⁺ calcd.: 785.4, found 785.3.

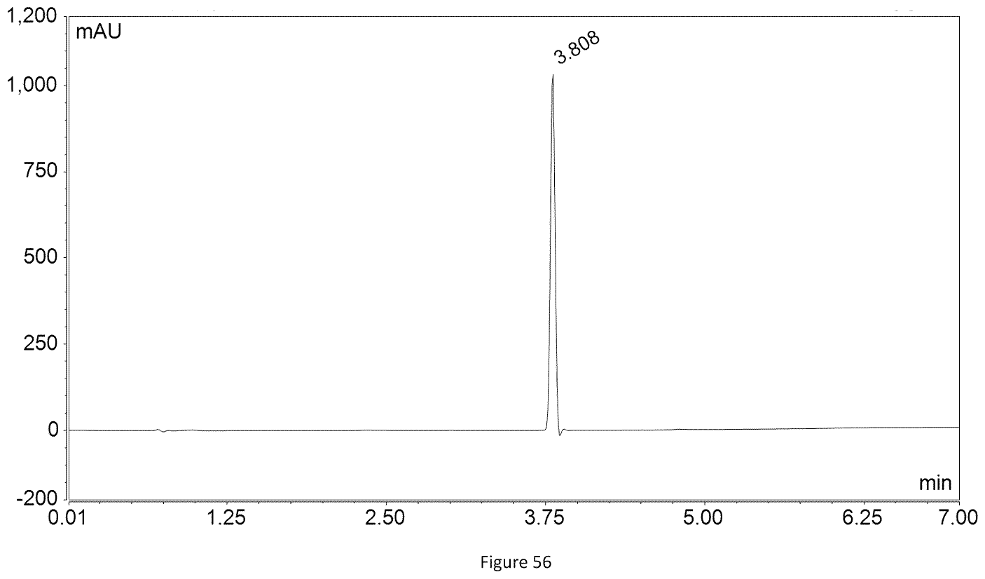

FIG. 56 shows an HPLC/UV chromatogram of the compound O-(5-tert.-butoxy-carbonyl-aminopentyl)-N-(L-alanine-tert.-butylester)-O—(OTS-964)-Phosphoramidate.

### O-(5-tert.-butoxy-carbonyl-aminopentyl)-N-(L-alanine-tert.-butylester)-O-(Ganetespib)-Phosphoramidate

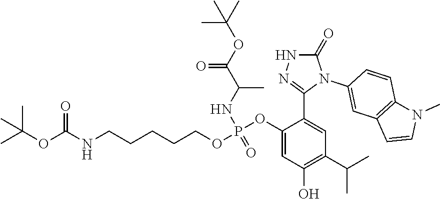 [SMILES: CC(NP(=O)(OCCCCCNC(=O)OC(C)(C)C)Oc1cc(O)c(C(C)C)cc1-c1n[nH]c(=O)n1-c1ccc2c(ccn2C)c1)C(=O)OC(C)(C)C]

The title compound was synthesized in accordance to general Method 1 from 10 mg Ganetespib (0.028 mmol). The product was obtained as solid after preparative HPLC (Method C) and lyophilization. (5 mg, 0.007 mmol, 25%). MS for C₃₇H₅₄N₆O₉P⁺ [M+H]⁺ calcd.:

757.4 found 757.3.

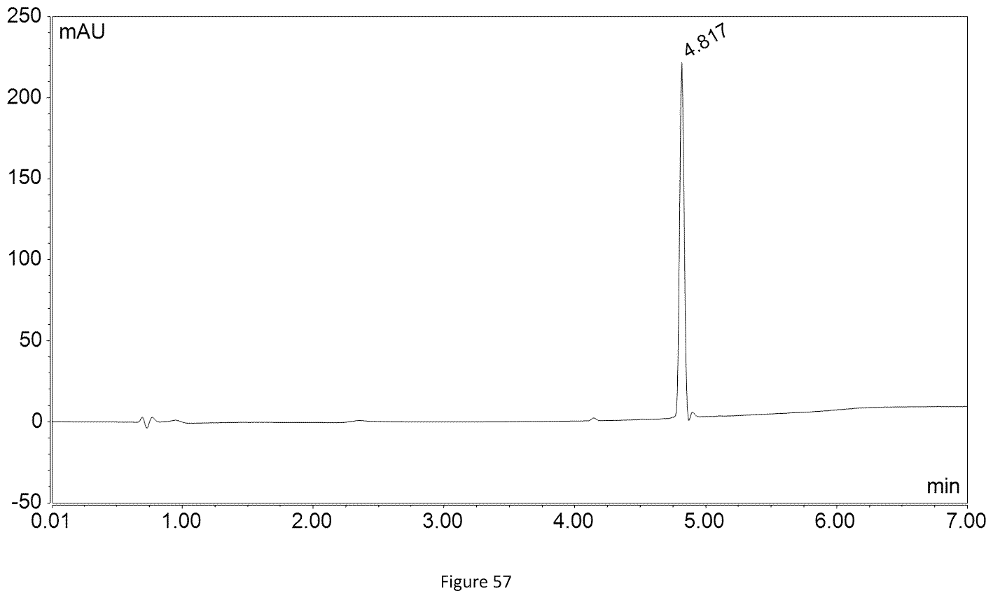

FIG. 57 shows an HPLC/UV chromatogram of the compound O-(5-tert.-butoxy-carbonyl-aminopentyl)-N-(L-alanine-tert.-butylester)-O-(Ganetespib)-Phosphoramidate.

### O-(5-tert.-butoxy-carbonyl-aminopentyl)-N-(L-alanine-tert.-butylester)-O-(Birabresib)-Phosphoramidate

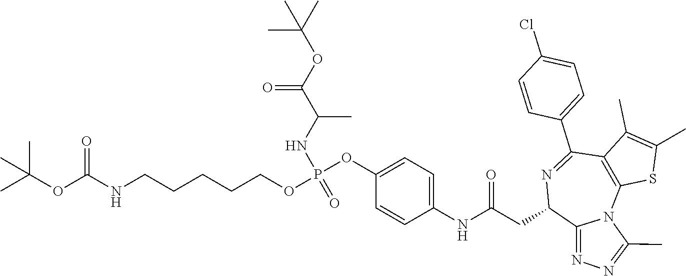 [SMILES: Cc1sc2c(c1C)C(c1ccc(Cl)cc1)=N[C@@H](CC(=O)Nc1ccc(OP(=O)(NC(C)C(=O)OC(C)(C)C)OCCCCCNC(=O)OC(C)(C)C)cc1)c1nnc(C)n1-2]

The title compound was synthesized in accordance to general Method 1 from 10 mg Birabresib (0.020 mmol). The product was obtained as white solid after preparative HPLC (Method C) and lyophilization. (15.3 mg, 0.0172 mmol, 86%). MS for C₄₂H₅₆ClN₇O₈PS+[M+H]⁺ calcd.: 884.3 found 884.3.

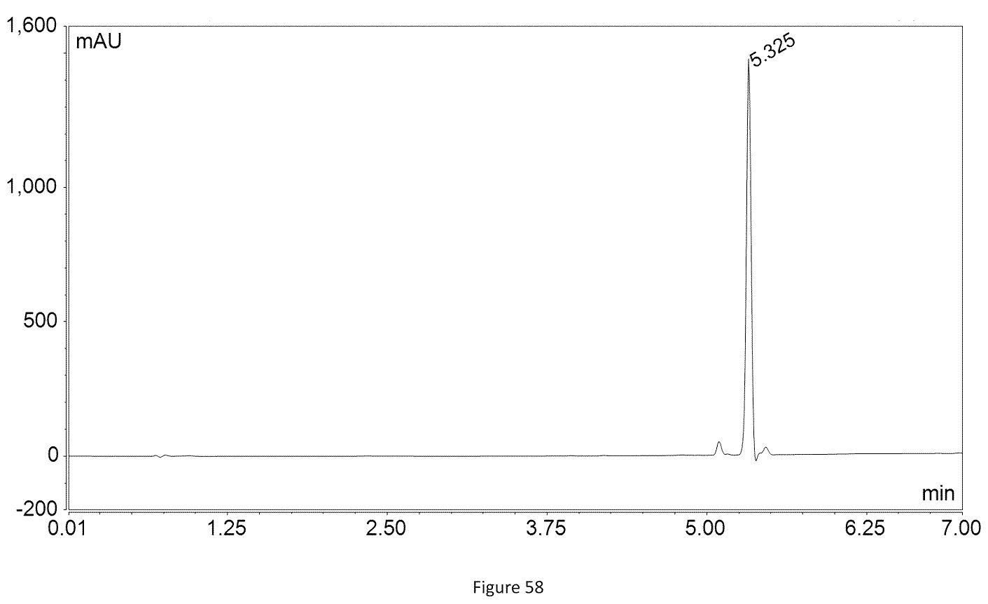

FIG. 58 shows an HPLC/UV chromatogram of the compound O-(5-tert.-butoxy-carbonyl-aminopentyl)-N-(L-alanine-tert.-butylester)-O-(Birabresib)-Phosphoramidate.

### O-(5-tert.-butoxy-carbonyl-aminopentyl)-N-(L-alanine-L-alanine-tert.-butyl)-O—(SNX-2112)-Phosphoramidate

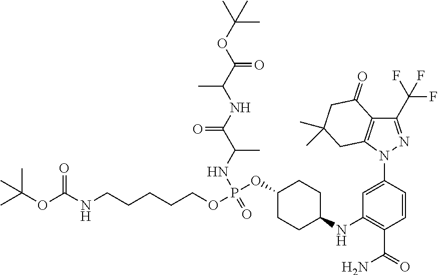 [SMILES: CC(NP(=O)(OCCCCCNC(=O)OC(C)(C)C)O[C@H]1CC[C@H](Nc2cc(-n3nc(C(F)(F)F)c4c3CC(C)(C)CC4=O)ccc2C(N)=O)CC1)C(=O)NC(C)C(=O)OC(C)(C)C]

The title compound was synthesized in accordance to general Method 1 from 11.5 mg SNX-2112 (0.0248 mmol). The product was obtained as white solid after preparative HPLC (Method C) and lyophilization. (6.1 mg, 0.0066 mmol, 26.5%). HR-MS for C₄₃H₆₆F₃N₇O₁₀P⁺ [M+H]⁺ calcd.: 928.4555 found 928.46185.

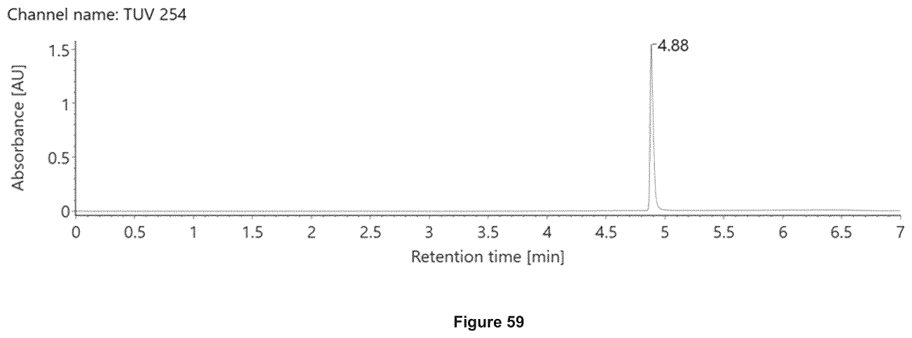

FIG. 59 shows an HPLC/UV chromatogram of the compound O-(5-tert.-butoxy-carbonyl-aminopentyl)-N-(L-alanine-L-alanine-tert.-butyl)-O—(SNX-2112)-Phosphoramidate.

### O-(5-tert.-butoxy-carbonyl-aminopentyl)-N-(L-alanine-L-alanine-tert.-butyl)-O-(Gemcitabine)

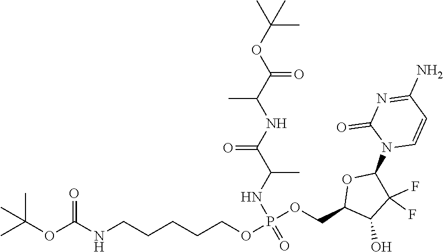 [SMILES: CC(NP(=O)(OCCCCCNC(=O)OC(C)(C)C)OC[C@H]1O[C@@H](n2ccc(N)nc2=O)C(F)(F)[C@@H]1O)C(=O)NC(C)C(=O)OC(C)(C)C]

The title compound was synthesized in accordance to general Method 1 from 50 mg Gemcitabine (0.19 mmol). The product was obtained as white solid after preparative HPLC (Method C) and lyophilization. (8 mg, 0.01 mmol, 5.8%). MS for C₂₉H₅₀F₂N₆O₁₁P⁺ [M+H]⁺ calcd.: 727.3 found 727.2.

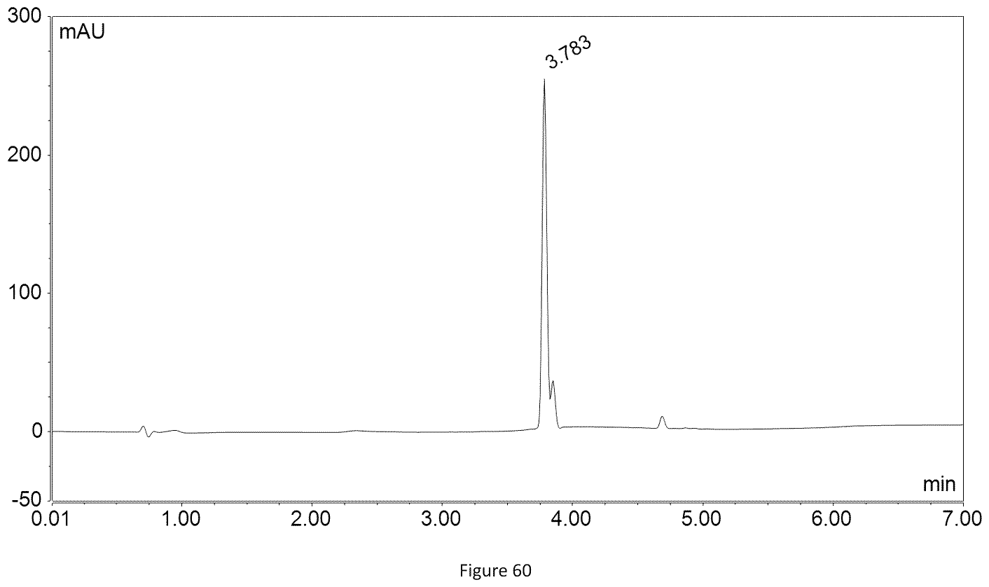

FIG. 60 shows an HPLC/UV chromatogram of the compound O-(5-tert.-butoxy-carbonyl-aminopentyl)-N-(L-alanine-L-alanine-tert.-butyl)-O-(Gemcitabine)-Phosphoramidate.

### O-(5-tert.-butoxy-carbonyl-aminopentyl)-N-(L-alanine-L-alanine-tert.-butyl)-O-(Barasertib)-Phosphoramidate

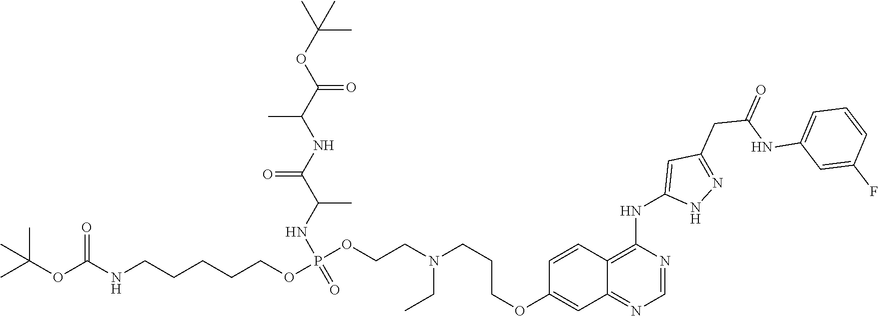 [SMILES: CCN(CCCOc1ccc2c(Nc3cc(CC(=O)Nc4cccc(F)c4)n[nH]3)ncnc2c1)CCOP(=O)(NC(C)C(=O)NC(C)C(=O)OC(C)(C)C)OCCCCCNC(=O)OC(C)(C)C]

The title compound was synthesized in accordance to general Method 1 from 5 mg Barasertib (0.01 mmol). The product was obtained as white solid after preparative HPLC (Method C) and lyophilization. (9.5 mg, 0.0097 mmol, 97%). HR-MS for C₄₆H₆₉FN₁₀O₁₀P⁺ [M+H]⁺ calcd.: 971.4914 found 971.41.

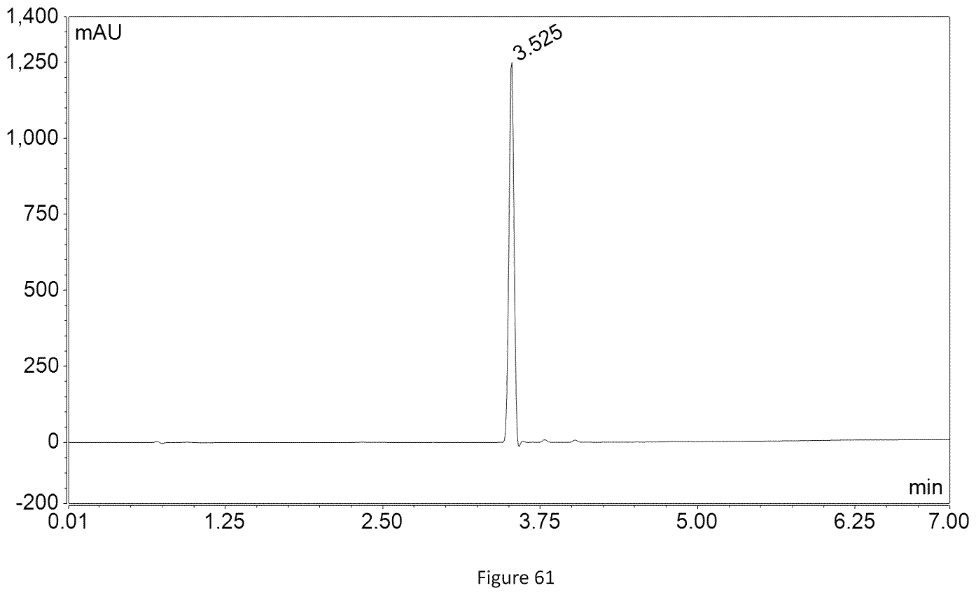

FIG. 61 shows an HPLC/UV chromatogram of the compound O-(5-tert.-butoxy-carbonyl-aminopentyl)-N-(L-alanine-L-alanine-tert.-butyl)-O-(Barasertib)-Phosphoramidate.

### O—(P5(PEG24)-amidopentyl)-N-(L-alanine-tert.-butylester)-O-(DxD)-Phosphoramidate

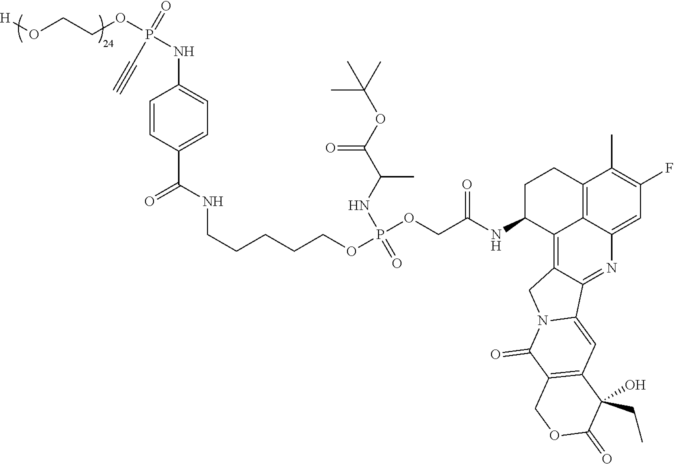

The title compound was synthesized in accordance to general Method 1 from 8.3 mg of O-(5-tert.-butoxy-carbonyl-aminopentyl)-N-(L-alanine-tert.-butylester)-O-(Dxd)-Phosphoramidate (0.009 mmol, 1 eq). The product was obtained as yellowish oil after preparative HPLC (Method C) and lyophilization. (13.3 mg, 0.007 mmol, 77% over two steps). HR-MS for C₉₅H₁₅₃FN₆O₃₇P₂²⁺ [M+2H]²⁺ calcd.: 1025.4862, found 1025.4873

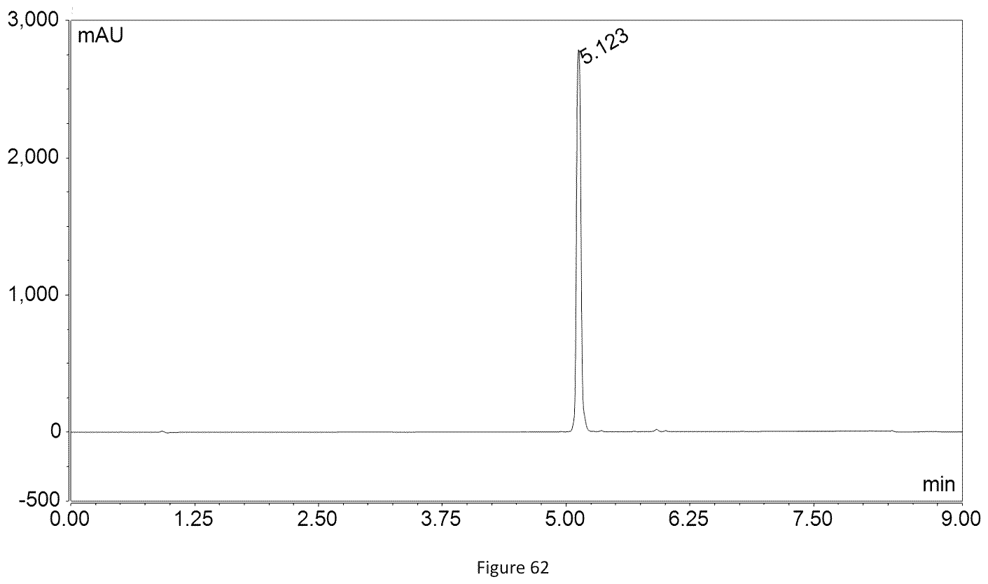

FIG. 62 shows an HPLC/UV chromatogram of the compound O—(P5(PEG24)-amidopentyl)-N-(L-alanine-tert.-butylester)-O-(DxD)-Phosphoramidate.

### O—(P5(PEG24)-amidopentyl)-N-(L-alanine-L-alanine-COOH)—O-(DxD)-Phosphoramidate

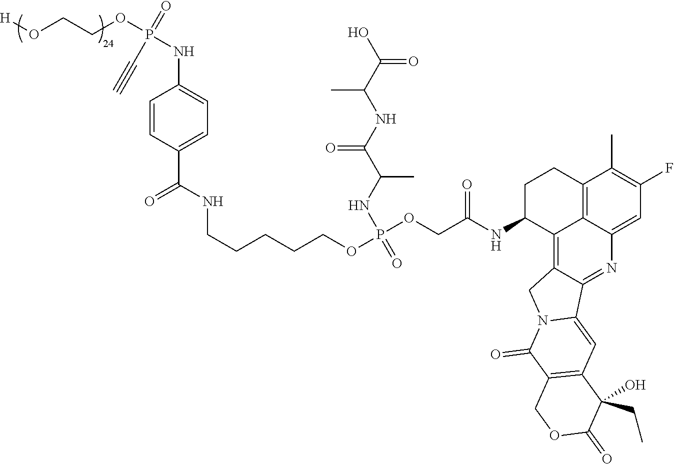

4.4 mg of O-(5-tert.-butoxy-carbonyl-aminopentyl)-N-(L-alanine-L-alanine-tert.-butylester)-O-(DxD)-Phosphoramidate (0.006 mmol) were dissolved in 0.5 mL of 50% TFA in DCM containing 5% water and cooled with wet ice. The reaction was stirred for 45 minutes on ice. The solvents were removed in nitrogen stream and the residue was dried under high vacuum at a Schlenk-line. The residue was dissolved in 100 μL DMSO and incubated with 41 μL of P5-PEG24-COOH (0.2 M in DMSO, 0.008 mmol, 1.5 eq), 33 μL PyBOP (0.2 M in DMSO, 0.007 mmol, 1.2 eq) and 10 μL DIPEA (0.055 mmol, 10 eq). The reaction was stirred for 1 h at room temperature. Subsequently, the reaction was diluted with H₂O/MeCN (1:1) and purified by HPLC (Method C). 7.2 mg of the title compound were isolated (3.5 μmol, 58%). HR-MS for C₉₄H₁₅₀FN₇O₃₈P₂²⁺ [M+2H]²⁺ calcd.: 1032.9735, found 1032.9683.

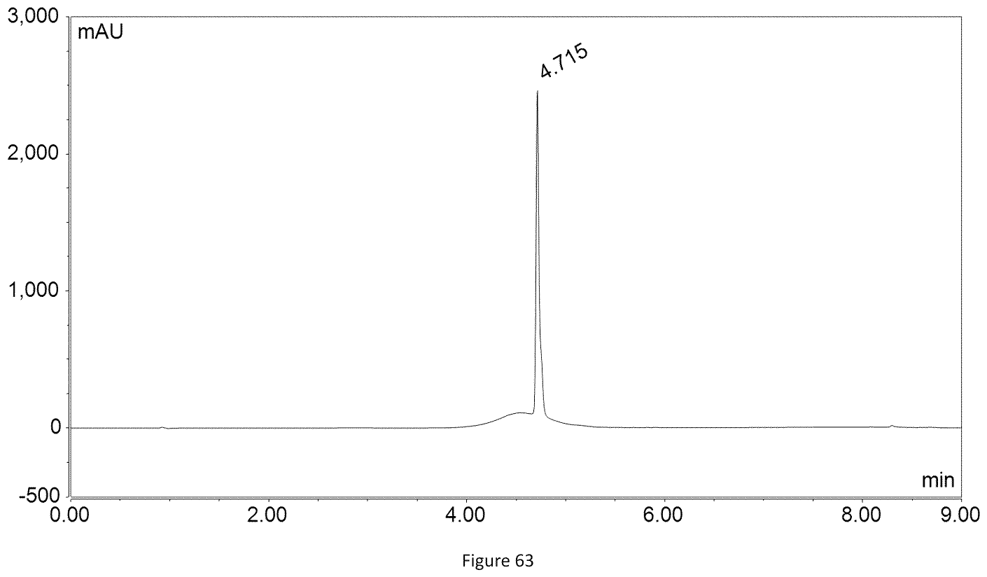

FIG. 63 shows an HPLC/UV chromatogram of the compound O—(P5(PEG24)-amidopentyl)-N-(L-alanine-L-alanine-COOH)—O-(DxD)-Phosphoramidate.

### O-(6-Maleimidocaproic acid-amidopentyl)-N-(L-alanine-L-alanine-COOH)—O-(DxD)-Phosphoramidate

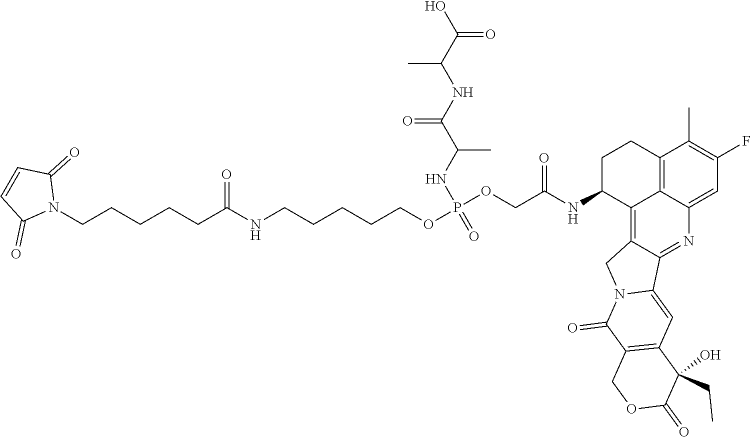 [SMILES: CC[C@@]1(O)C(=O)OCc2c1cc1n(c2=O)Cc2c-1nc1cc(F)c(C)c3c1c2[C@@H](NC(=O)COP(=O)(NC(C)C(=O)NC(C)C(=O)O)OCCCCCNC(=O)CCCCCN1C(=O)C=CC1=O)CC3]

3 mg of O-(5-tert.-butoxy-carbonyl-aminopentyl)-N-(L-alanine-L-alanine-tert.-butylester)-O-(DxD)-Phosphoramidate (0.003 mmol) were dissolved in 0.5 mL of 50% TFA in DCM containing 5% water and cooled with wet ice. The reaction was stirred for 45 minutes on ice. The solvents were removed in nitrogen stream and the residue was dried under high vacuum at a Schlenk-line. The residue was dissolved in 100 μL DMSO and 2 mg 6-Maleimidocaproic acid NHS-ester (0.006 mmol, 1.5 eq) and 11 μL DIPEA (0.06 mmol, 20 eq) were added. The reaction was stirred for 30 minutes at room temperature. Subsequently, the reaction was diluted with H₂O/MeCN (1:1) and purified by HPLC (Method C). 1 mg of the title compound were isolated (1 μmol, 33%). MS for C₄₇H₅₈FN₇O₁₄P⁺ [M+H]⁺ calcd.: 994.4, found

994.3.

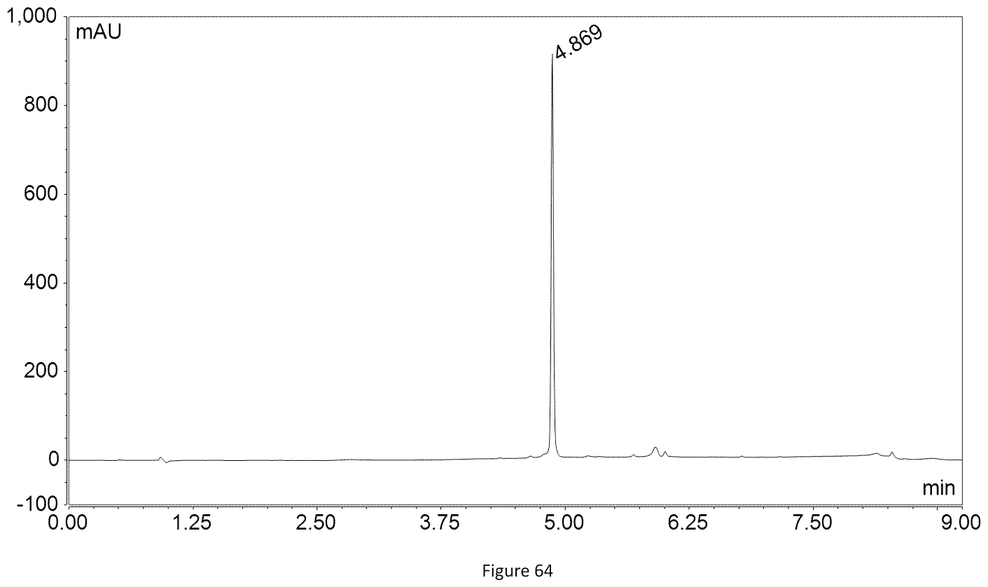

FIG. 64 shows an HPLC/UV chromatogram of the compound O-(6-Maleimidocaproic acid-amidopentyl)-N-(L-alanine-L-alanine-COOH)—O-(DxD)-Phosphoramidate.

### O-(lodoaceticacid-amidopentyl)-N-(L-alanine-L-alanine-COOH)—O-(DxD)-Phosphoramidate

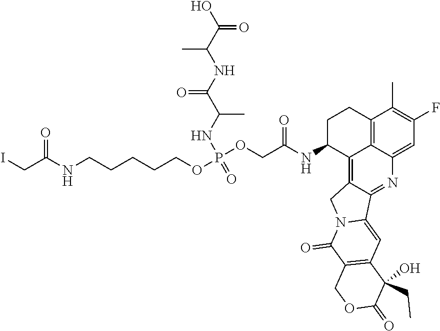 [SMILES: CC[C@@]1(O)C(=O)OCc2c1cc1n(c2=O)Cc2c-1nc1cc(F)c(C)c3c1c2[C@@H](NC(=O)COP(=O)(NC(C)C(=O)NC(C)C(=O)O)OCCCCCNC(=O)CI)CC3]

3.5 mg of O-(5-tert.-butoxy-carbonyl-aminopentyl)-N-(L-alanine-L-alanine-tert.-butylester)-O-(DxD)-Phosphoramidate (0.003 mmol) were dissolved in 0.5 mL of 50% TFA in DCM containing 5% water and cooled with wet ice. The reaction was stirred for 45 minutes on ice. The solvents were removed in nitrogen stream and the residue was dried under high vacuum at a Schlenk-line. The residue was dissolved in 100 μL DMSO and 2.1 mg iodoacetic acid NHS-ester (0.007 mmol, 2 eq) and 6.4 μL DIPEA (0.04 mmol, 10 eq) were added. The reaction was stirred for 30 minutes at room temperature. Subsequently, the reaction was diluted with H₂O/MeCN (1:1) and purified by HPLC (Method C). 1 mg of the title compound were isolated (1 μmol, 33%). MS for C₃₉H₄₈FIN₆O₁₂P⁺ [M+H]⁺ calcd.: 969.2, found

969.2.

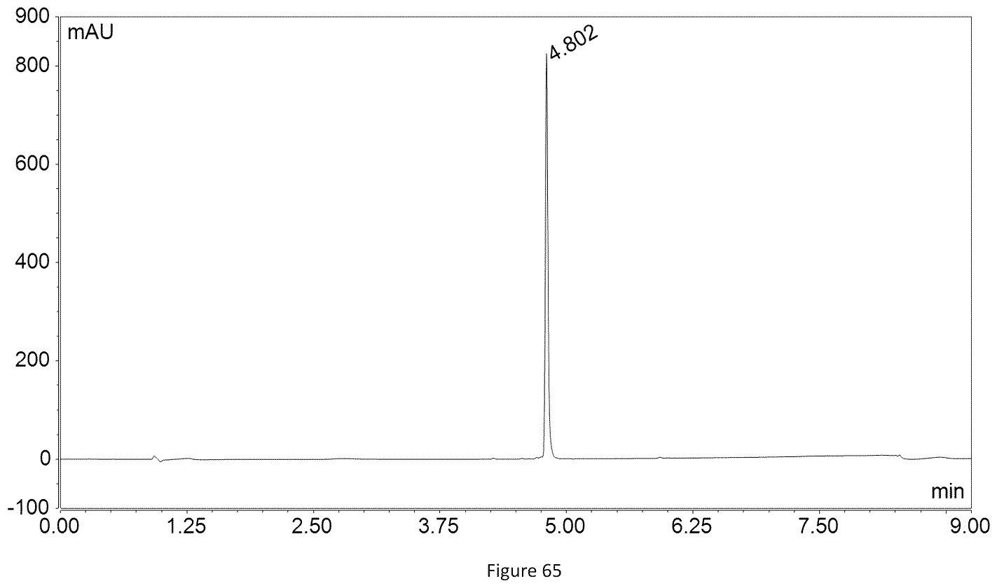

FIG. 65 shows an HPLC/UV chromatogram of the compound O-(lodoaceticacid-amidopentyl)-N-(L-alanine-L-alanine-COOH)—O-(DxD)-Phosphoramidate.

### O—(P5(PEG24)-amidopentyl)-N—(B3-alanine-L-alanine-COOH)—O-(DxD)-Phosphoramidate

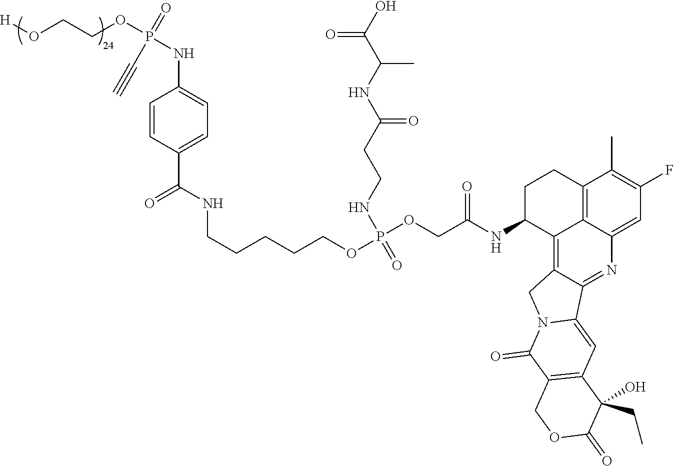

1 mg of O-(5-tert.-butoxy-carbonyl-aminopentyl)-N—(β-alanine-L-alanine-tert.-butylester)-O-(DxD)-Phosphoramidate (0.001 mmol) were dissolved in 0.4 mL of 50% TFA in DCM containing 5% water and cooled with wet ice. The reaction was stirred for 3 h on ice. The solvents were removed in nitrogen stream and the residue was dried under high vacuum at a Schlenk-line. The residue was dissolved in 100 μL DMSO and incubated with 9 μL of P5-PEG24-COOH (0.2 M in DMSO, 0.002 mmol, 1.5 eq), 7 μL PyBOP (0.2 M in DMSO, 0.0013 mmol, 1.2 eq) and 4 μL DIPEA (0.0225 mmol, 20 eq). The reaction was stirred for 1 h at room temperature. Subsequently, the reaction was diluted with H2O/MeCN (1:1) and purified by HPLC (Method C). 1.2 mg of the title compound were isolated (0.58 μmol, 58%). HR-MS for C₉₄H₁₅₀FN₇O₃₈P₂²⁺ [M+2H]²⁺ calcd.: 1032.9735, found 1032.9686.

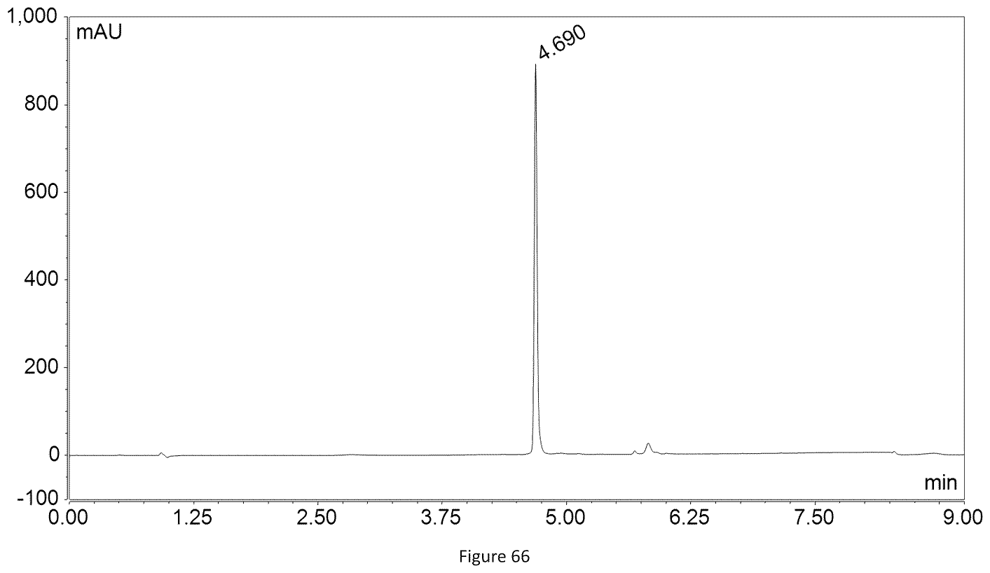

FIG. 66 shows an HPLC/UV chromatogram of the compound O—(P5(PEG24)-amidopentyl)-N—(β-alanine-L-alanine-COOH)—O-(DxD)-Phosphoramidate.

### O—(P5(PEG24)-amidopentyl)-N-(γ-Aminobutyric acid-L-alanine-COOH)—O-(DxD)-Phosphoramidate

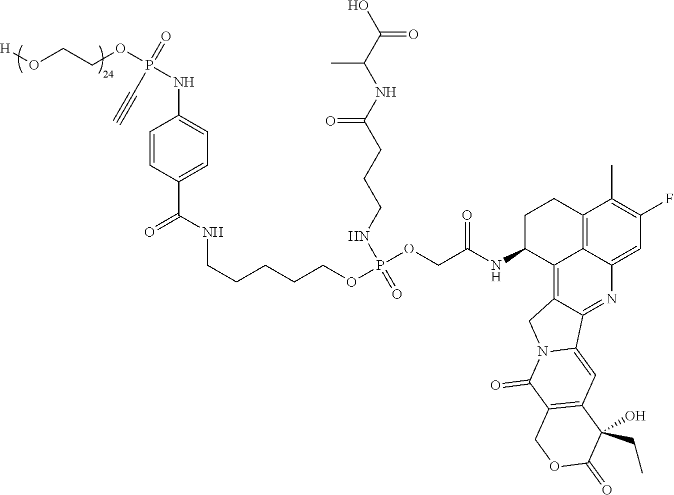

3.3 mg of O-(5-tert.-butoxy-carbonyl-aminopentyl)-N-(γ-Aminobutyric acid-L-alanine-tert.-butylester)-O-(DxD)-Phosphoramidate (0.004 mmol) were dissolved in 0.4 mL of 50% TFA in DCM containing 5% water and cooled with wet ice. The reaction was stirred for 3 h on ice. The solvents were removed in nitrogen stream and the residue was dried under high vacuum at a Schlenk-line. The residue was dissolved in 100 μL DMSO and incubated with 30 μL of P5-PEG24-COOH (0.2 M in DMSO, 0.006 mmol, 1.5 eq), 24 μL PyBOP (0.2 M in DMSO, 0.005 mmol, 1.2 eq) and 7 μL DIPEA (0.04 mmol, 10 eq). The reaction was stirred for 1 h at room temperature. Subsequently, the reaction was diluted with H2O/MeCN (1:1) and purified by HPLC (Method C). 5.6 mg of the title compound were isolated (2.7 μmol, 67%). HR-MS for C₉₅H₁₅₂FN₇O₃₈P₂²⁺ [M+2H]²⁺ calcd.: 1039.9813, found 1039.9763.

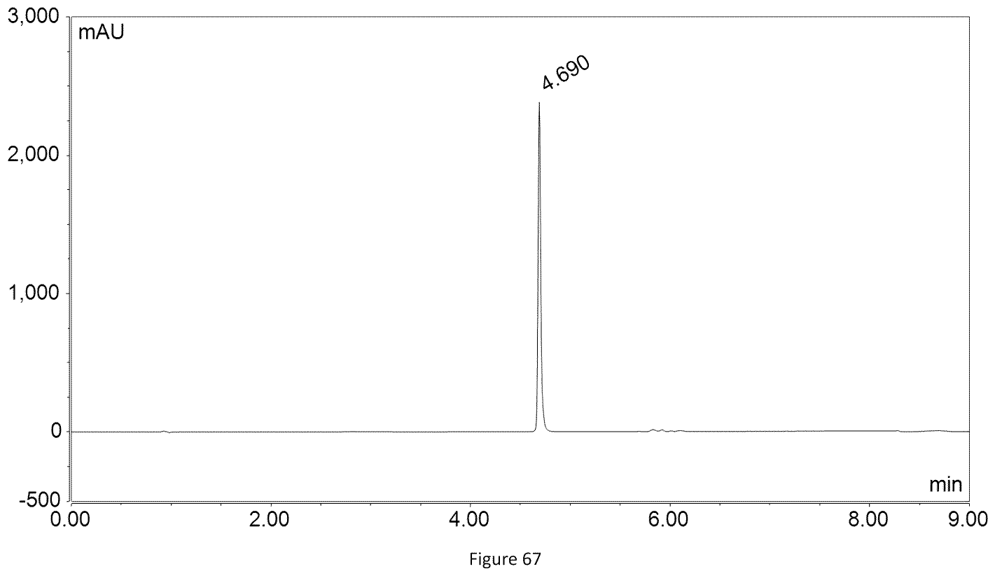

FIG. 67 shows an HPLC/UV chromatogram of the compound O—(P5(PEG24)-amidopentyl)-N-(γ-Aminobutyric acid-L-alanine-COOH)—O-(DxD)-Phosphoramidate.

### O—(P5(PEG24)-amidopentyl)-N-(5-Aminovaleric acid-L-alanine-COOH)—O-(DxD)-Phosphoramidate

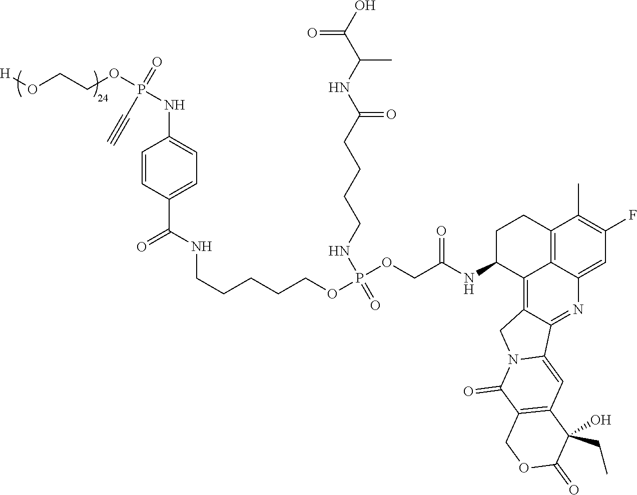

4.6 mg of O-(5-tert.-butoxy-carbonyl-aminopentyl)-N-(5-Aminovaleric acid-L-alanine-tert.-butylester)-O-(DxD)-Phosphoramidate (0.005 mmol) were dissolved in 0.4 mL of 50% TFA in DCM containing 5% water and cooled with wet ice. The reaction was stirred for 3 h on ice. The solvents were removed in nitrogen stream and the residue was dried under high vacuum at a Schlenk-line. The residue was dissolved in 100 μL DMSO and incubated with 42 μL of P5-PEG24-COOH (0.2 M in DMSO, 0.008 mmol, 1.5 eq), 33 μL PyBOP (0.2 M in DMSO, 0.007 mmol, 1.2 eq) and 10 μL DIPEA (0.05 mmol, 10 eq). The reaction was stirred for 1 h at room temperature. Subsequently, the reaction was diluted with H2O/MeCN (1:1) and purified by HPLC (Method C). 9.7 mg of the title compound were isolated (4.6 μmol, 90%). HR-MS for C₉₆H₁₅₄FN₇O₃₈P₂²⁺ [M+2H]²⁺ calcd.: 1046.9891, found 1046.9886.

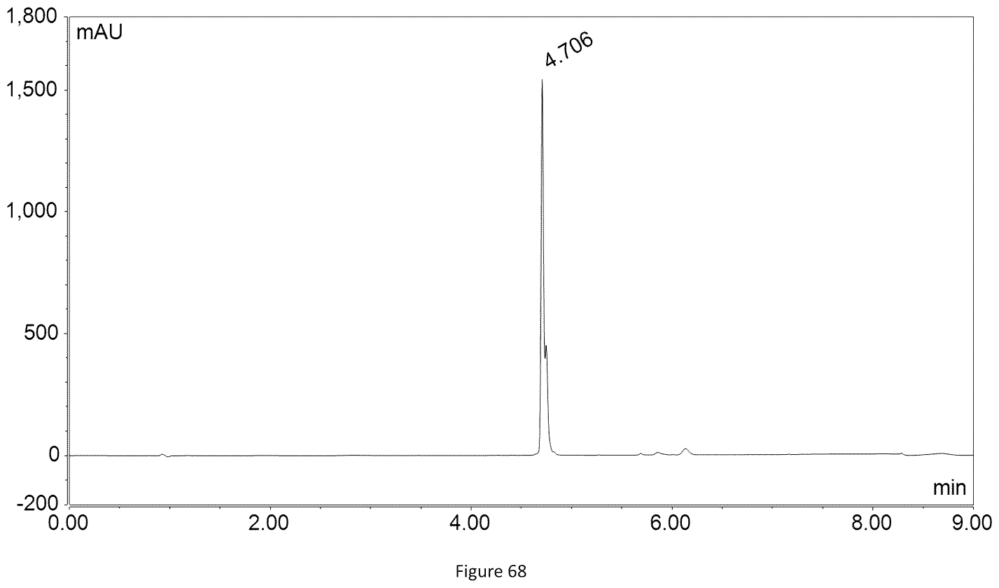

FIG. 68 shows an HPLC/UV chromatogram of the compound O—(P5(PEG24)-amidopentyl)-N-(5-Aminovaleric acid-L-alanine-COOH)—O-(DxD)-Phosphoramidate.

### O—(P5(PEG24)-amidopentyl)-N-(L-alanine-tert.-butylester)-O—(OTS-964)-Phosphoramidate

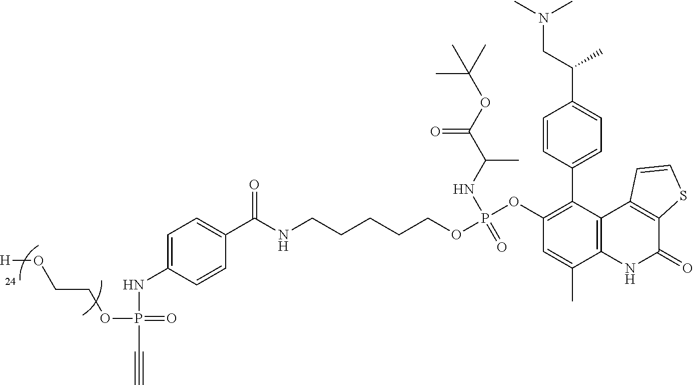

The title compound was synthesized in accordance to general Method 1 from 8.8 mg of O-(5-tert.-butoxy-carbonyl-aminopentyl)-N-(L-alanine-tert.-butylester)-O—(OTS-964)-Phosphoramidate (0.011 mmol, 1 eq). The product was obtained as yellowish oil after preparative HPLC (Method C) and lyophilization. (19.4 mg, 0.010 mmol, 91% over two steps). MS for C₉₂H₁₅₃N₅O₃₃P₂S²⁺[M+2H]²⁺ calcd.: 974.98, found 975.10.

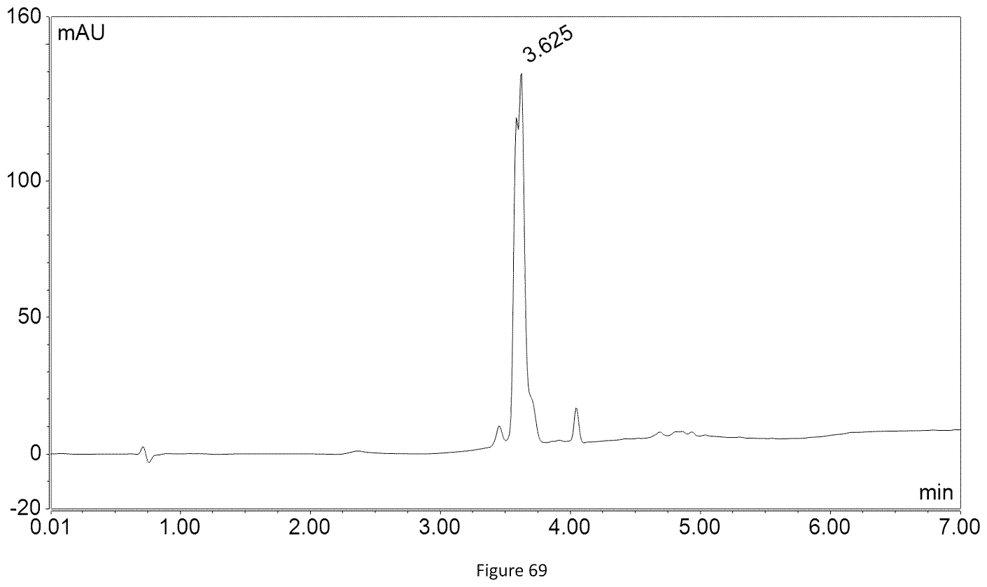

FIG. 69 shows an HPLC/UV chromatogram of the compound O—(P5(PEG24)-amidopentyl)-N-(L-alanine-tert.-butylester)-O—(OTS-964)-Phosphoramidate.

### O—(P5(PEG24)-amidopentyl)-N-(L-alanine-tert.-butylester)-O-(Ganetespib)-Phosphoramidate

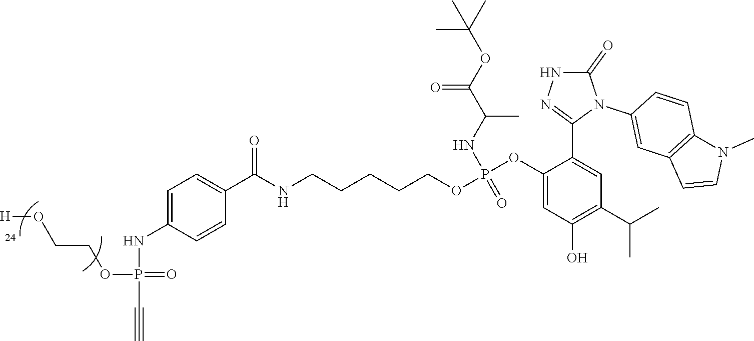

The title compound was synthesized in accordance to general Method 1 from mg of O-(5-tert.-butoxy-carbonyl-aminopentyl)-N-(L-alanine-tert.-butylester)-O-(Ganetespib)-Phosphoramidate (0.007 mmol, 1 eq). The product was obtained as yellowish oil after preparative HPLC (Method C) and lyophilization. (5.5 mg, 0.0028 mmol, 40% over two steps). MS for C₈₉H₁₄₉N₇O₃₄P₂²⁺ [M+2H]²⁺ calcd.: 960.98, found 961.0.

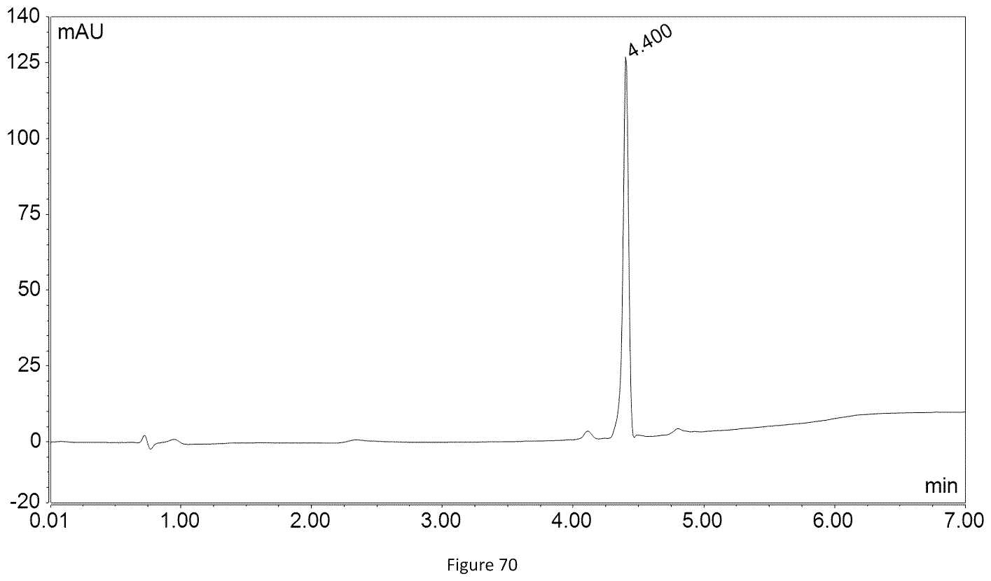

FIG. 70 shows an HPLC/UV chromatogram of the compound O—(P5(PEG24)-amidopentyl)-N-(L-alanine-tert.-butylester)-O-(Ganetespib)-Phosphoramidate.

### O—(P5(PEG24)-amidopentyl)-N-(L-alanine-tert.-butylester)-O-(Birabresib)-Phosphoramidate

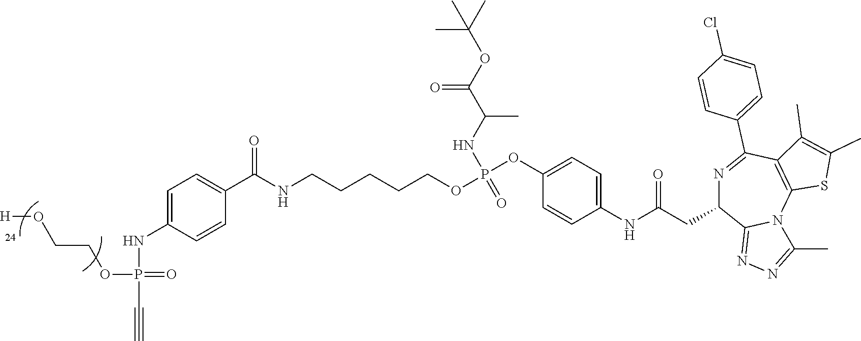

The title compound was synthesized in accordance to general Method 1 from 11.8 mg of O-(5-tert.-butoxy-carbonyl-aminopentyl)-N-(L-alanine-tert.-butylester)-O-(Birabresib)-Phosphoramidate (0.008 mmol, 1 eq). The product was obtained as yellowish oil after preparative HPLC (Method C) and lyophilization. (5.5 mg, 0.0027 mmol, 33.8% over two steps). MS for C₉₄H₁₅₁ClN₈O₃₃P₂S²⁺[M+2H]²⁺ calcd.: 1025.0, found 1024.5.

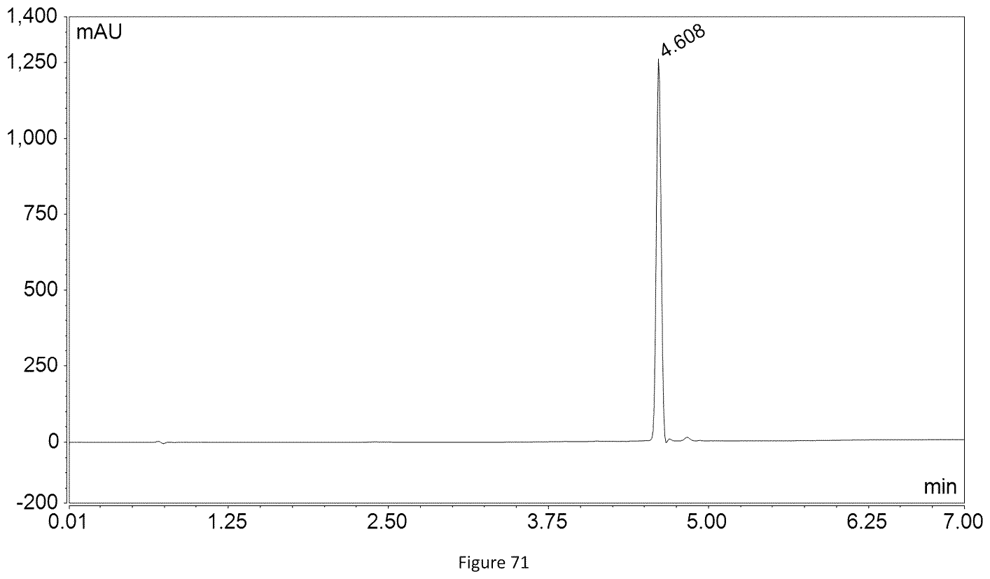

FIG. 71 shows an HPLC/UV chromatogram of the compound O—(P5(PEG24)-amidopentyl)-N-(L-alanine-tert.-butylester)-O-(Birabresib)-Phosphoramidate.

### O—(P5(PEG24)-amidopentyl)-N-(L-alanine-L-alanine-tert.-butylester)-O—(SNX-2112)-Phosphoramidate

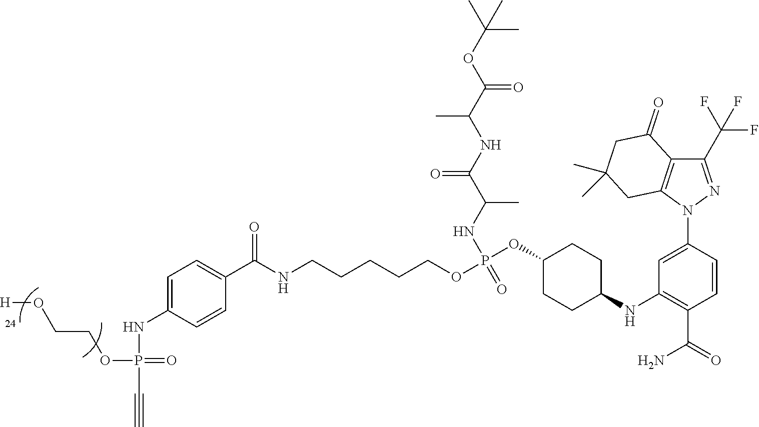

The title compound was synthesized in accordance to general Method 1 from 6.1 mg of O-(5-tert.-butoxy-carbonyl-aminopentyl)-N-(L-alanine-L-alanine-tert.-butylester)-O— (SNX-2112)-Phosphoramidate (0.0066 mmol, 1 eq). The product was obtained as yellowish oil after preparative HPLC (Method C) and lyophilization. (7.05 mg, 0.0034 mmol, 51.3% over two steps). MS for C₉₅H₁₆₁F₃N₈O₃₅P₂²⁺ [M+2H]²⁺ calcd.: 1047.0, found 1046.6.

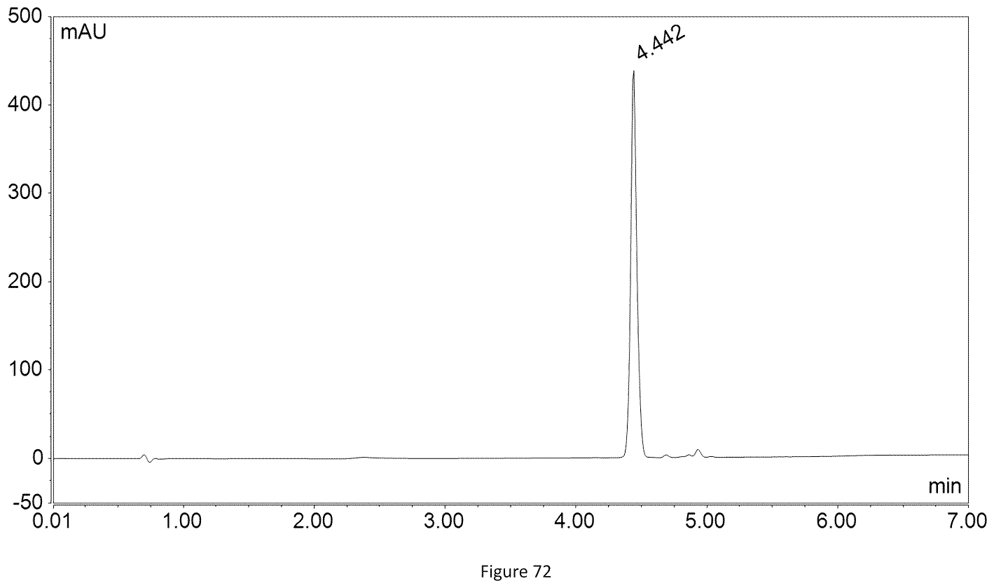

FIG. 72 shows an HPLC/UV chromatogram of the compound O—(P5(PEG24)-amidopentyl)-N-(L-alanine-L-alanine-tert.-butylester)-O—(SNX-2112)-Phosphoramidate.

### O—(P5(PEG24)-amidopentyl)-N-(L-alanine-L-alanine)-O—(SNX-2112)-Phosphoramidate

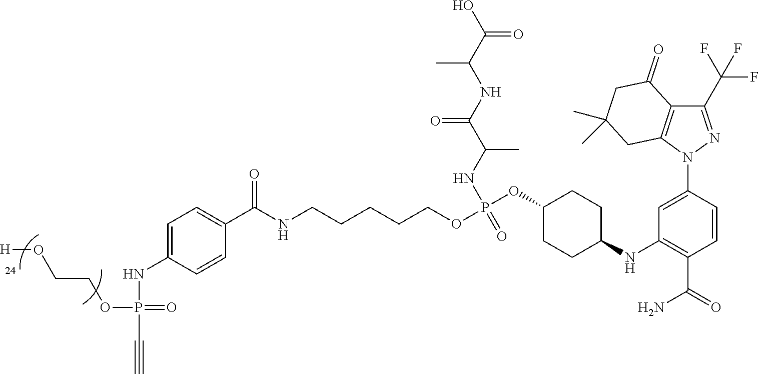

7.1 mg of O-(5-tert.-butoxy-carbonyl-aminopentyl)-N-(L-alanine-L-alanine-tert.-butylester)-O—(SNX-2112)-Phosphoramidate (0.0034 mmol) were dissolved in 0.5 mL of 50% TFA in DCM containing 5% water and cooled with wet ice. The reaction was stirred for 45 minutes on ice or until the Boc- and tBu-protecting groups were completely removed (followed by HPLC-MS). The solvents were removed in nitrogen stream and the residue was diluted with H₂O/MeCN (1:1) and purified by preparative HPLC (Method C). 2.7 mg of the title compound were isolated (1.4 μmol, 40%). MS for C₉₁H₁₅₃F₃N₈O₃₅P₂²⁺ [M+2H]²⁺ calcd.:

1018.5, found 1018.4.

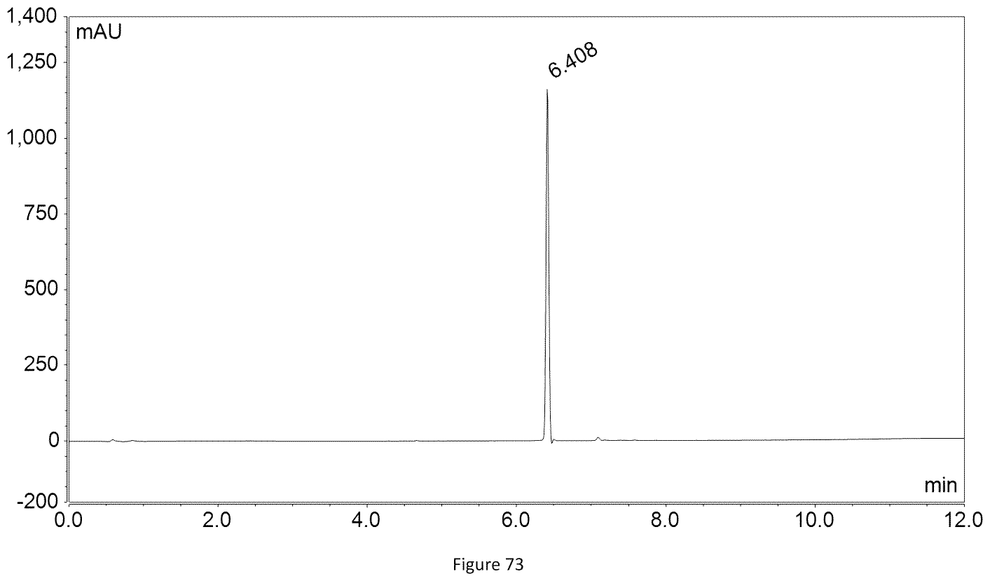

FIG. 73 shows an HPLC/UV chromatogram of the compound O—(P5(PEG24)-amidopentyl)-N—(L-alanine-L-alanine)-O—(SNX-2112)-Phosphoramidate.

### O—(P5(PEG24)-amidopentyl)-N—(L-alanine-L-alanine-tert.-butylester)-O-(Gemcitabine)-Phosphoramidate

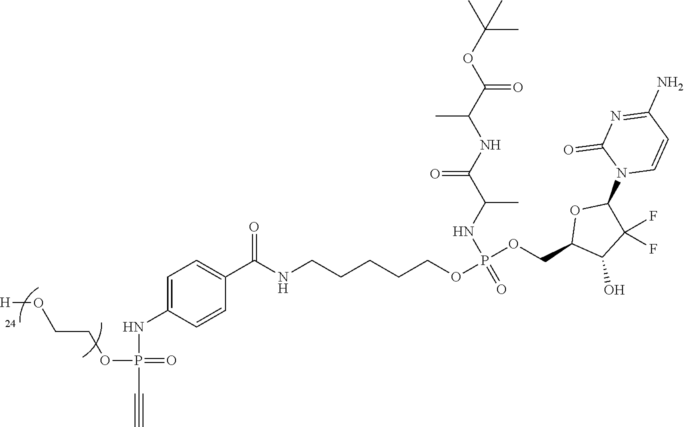

The title compound was synthesized in accordance to general Method 1 from 8 mg of O-(5-tert.-butoxy-carbonyl-aminopentyl)-N-(L-alanine-L-alanine-tert.-butylester)-O-(Gemcitabine)-Phosphoramidate (0.011 mmol, 1 eq). The product was obtained as yellowish oil after preparative HPLC (Method C) and lyophilization. (3.05 mg, 0.0016 mmol, 14.5% over two steps). MS for C₈₁H₁₄₅F₂N₇O₃₆P₂²⁺ [M+2H]²⁺ calcd.: 946.0, found 946.0.

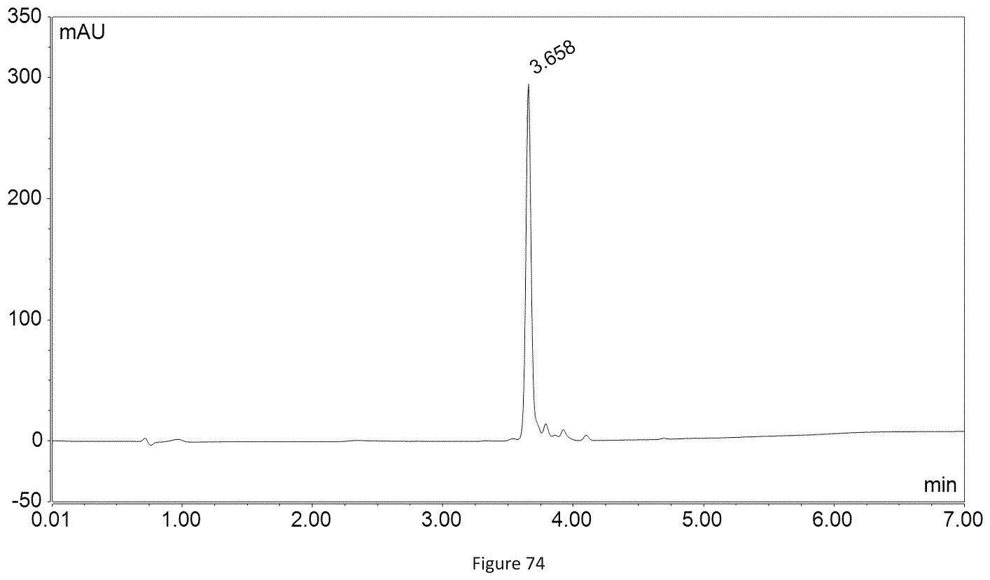

FIG. 74 shows an HPLC/UV chromatogram of the compound O—(P5(PEG24)-amidopentyl)-N—(L-alanine-L-alanine-tert.-butylester)-O-(Gemcitabine)-Phosphoramidate.

### O—(P5(PEG24)-amidopentyl)-N-(L-alanine-L-alanine)-O-(Gemcitabine)-Phosphoramidate

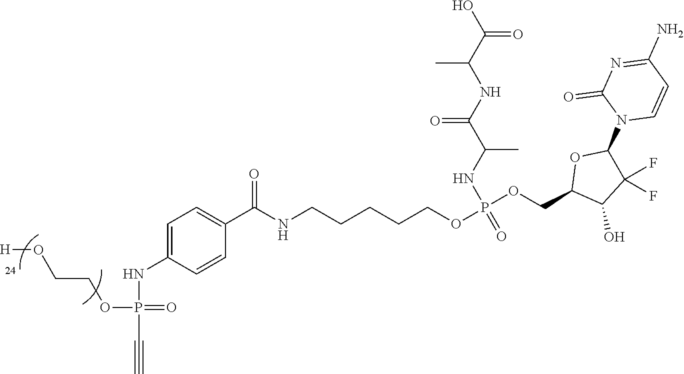

5 mg of O-(5-tert.-butoxy-carbonyl-aminopentyl)-N-(L-alanine-L-alanine-tert.-butylester)-O-(Gemcitabine)-Phosphoramidate (0.003 mmol) were dissolved in 0.5 mL of 50% TFA in DCM containing 5% water and cooled with wet ice. The reaction was stirred for 45 minutes on ice or until the Boc- and tBu-protecting groups were completely removed (followed by HPLC-MS). The solvents were removed in nitrogen stream and the residue was diluted with H₂O/MeCN (1:1) and purified by preparative HPLC (Method C). 4 mg of the title compound were isolated (2.2 μmol, 72.7%). MS for C₇₇H₁₃₇F₂N₇O₃₆P₂²⁺ [M+2H]²⁺ calcd.: 917.9, found 918.6.

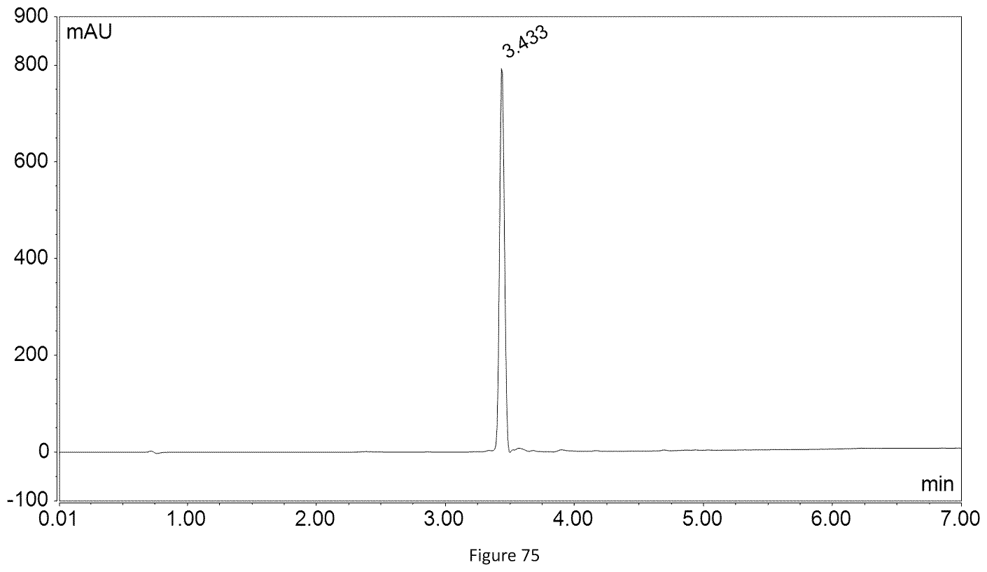

FIG. 75 shows an HPLC/UV chromatogram of the compound O—(P5(PEG24)-amidopentyl)-N—(L-alanine-L-alanine)-O-(Gemcitabine)-Phosphoramidate.

### O—(P5(PEG24)-amidopentyl)-N-(L-alanine-L-alanine-tert.-butylester)-O-(Barasertib)-Phosphoramidate

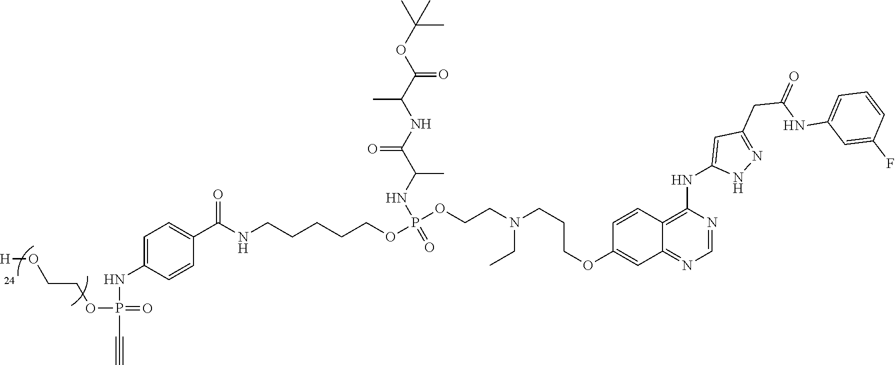

The title compound was synthesized in accordance to general Method 1 from 9.6 mg of O-(5-tert.-butoxy-carbonyl-aminopentyl)-N-(L-alanine-L-alanine-tert.-butylester)-O-(Barasertib)-Phosphoramidate (0.01 mmol, 1 eq). The product was obtained as yellowish oil after preparative HPLC (Method C) and lyophilization. (16 mg, 0.0075 mmol, 75% over two steps). MS for C₉₈H₁₆₄FN₁₁O₃₅P₂²⁺ [M+2H]²⁺ calcd.: 1068.54, found 1068.76.

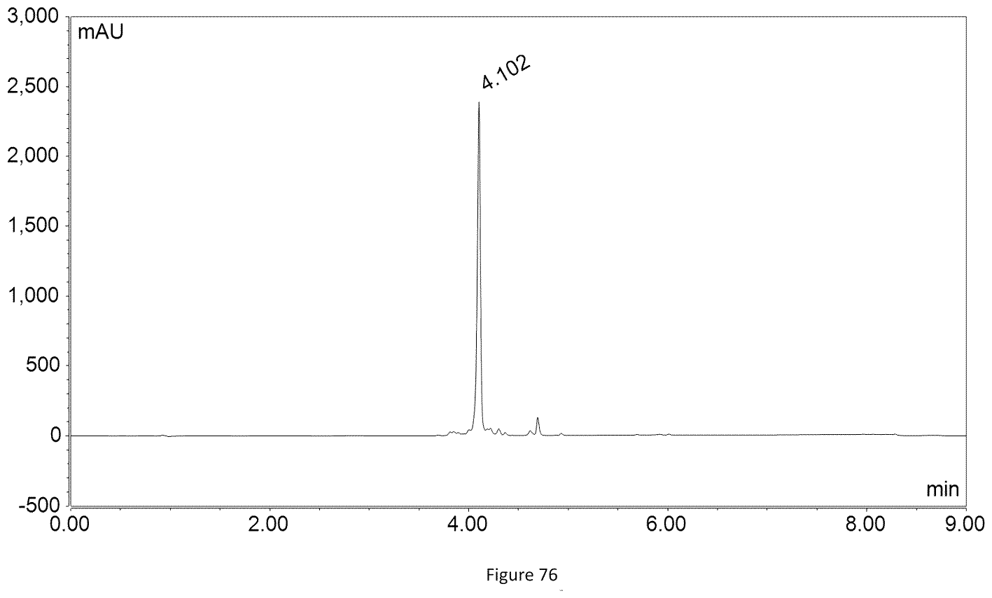

FIG. 76 shows an HPLC/UV chromatogram of the compound O—(P5(PEG24)-amidopentyl)-N—(L-alanine-L-alanine-tert.-butylester)-O-(Barasertib)-Phosphoramidate.

### O—(P5(PEG24)-amidopentyl)-N-(L-alanine-L-alanine)-O-(Barasertib)-Phosphoramidate

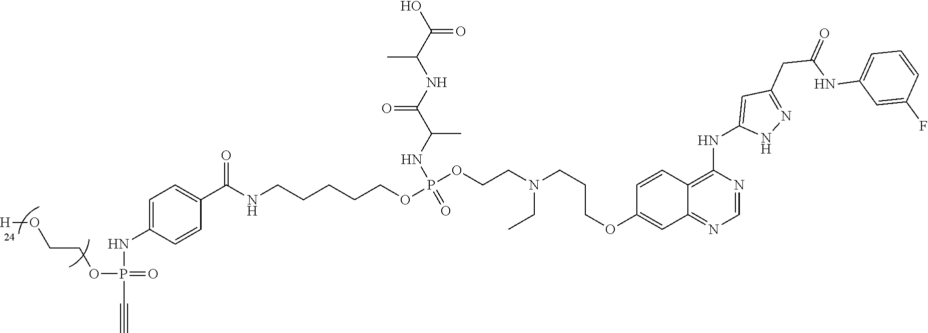

5.7 mg of O-(5-tert.-butoxy-carbonyl-aminopentyl)-N-(L-alanine-L-alanine-tert.-butylester)-O-(Barasertib)-Phosphoramidate (0.0027 mmol) were dissolved in 0.5 mL of 50% TFA in DCM containing 5% water and cooled with wet ice. The reaction was stirred for 45 minutes on ice or until the Boc- and tBu-protecting groups were completely removed (followed by HPLC-MS). The solvents were removed in nitrogen stream and the residue was diluted with H₂O/MeCN (1:1) and purified by preparative HPLC (Method C). 2.4 mg of the title compound were isolated (1.15 μmol, 42.6%). MS for C₉₄H₁₅₆FN₁₁O₃₅P₂²⁺ [M+2H]²⁺ calcd.: 1040.5, found 1040.0.

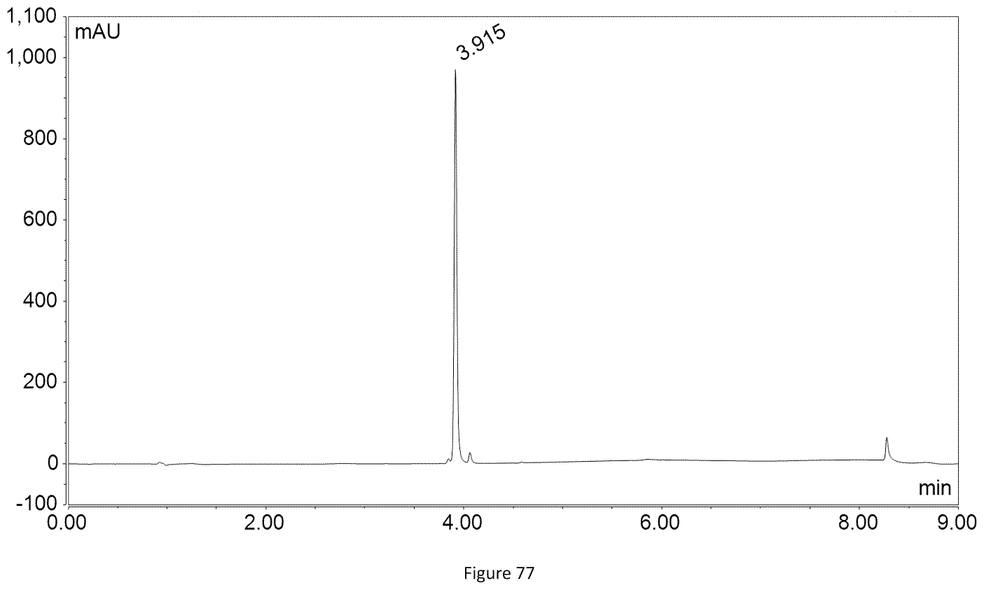

FIG. 77 shows an HPLC/UV chromatogram of the compound O—(P5(PEG24)-amidopentyl)-N—(L-alanine-L-alanine)-O-(Barasertib)-Phosphoramidate.

### Synthesis of P5(PEG24)-COOH

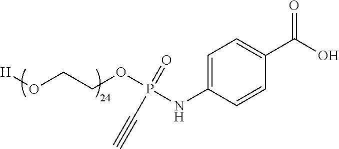

The title compound was synthesized in accordance with the following procedure. A 25 ml Schlenk tube with stirring bar was charged with 50 mg bis(diisopropylamino)chlorophosphine (187 μmol, 1.00 eq.) under a nitrogen atmosphere and cooled to 0° C. with wet ice. Slowly 450 μL ethynylmagnesium bromide solution (0.5 M in THF, 225 μmol, 1.20 eq.) were added. The cooling bath was removed after 5 minutes and the solution was allowed to stir at rt for 30 minutes. 36 mg tert-butyl-4-amiobenzoate (187 μmol, 1.00 eq) was dissolved in 0.5 ml of 1H-Tetrazole in Acetonitrile solution (0.45M, 1.2 eq.), added slowly to the reaction mixture, and stirred at rt for 30 minutes 201 mg of HO—PEG24-OH (187 μmol, 1.0 eq) was dissolved in 0.5 ml of 1H tetrazole in Acetonitrile solution (0.45M, 1,2 eq.), added to the reaction mixture slowly and stirred at rt for 30 minutes. A solution of hydrogen peroxide in water (0.1 ml, 30%) was added to the reaction mixture and stirred for five minutes. All volatiles were removed under reduced pressure, the obtained solid was dissolved in 2 ml TFA and stirred for 30 minutes. TFA was removed in a nitrogen stream and the product purified by preparative HPLC.

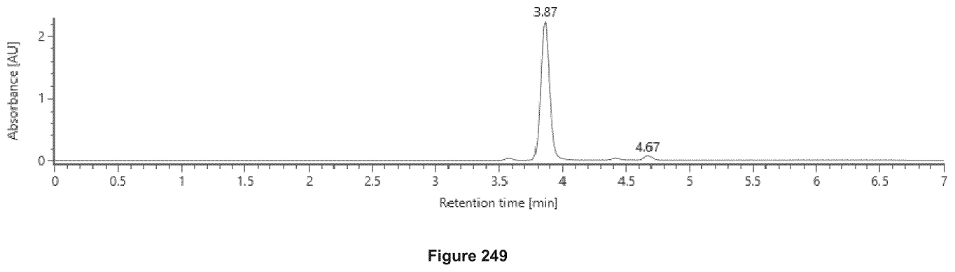

Preparative HPLC was performed on a BÜCHI Pure C-850 Flash-Prep system (BÜCHI Labortechnik A G, Switzerland) using a VP 250/21 Macherey-Nagel Nucleodur C18 HTec Spum column (Macherey-Nagel GmbH & Co. Kg, Germany) the following gradients: Method D: (A=H2O+0.1% TFA (trifluoroacetic acid), B=MeCN (acetonitrile)+0.1% TFA, flow rate 14 ml/min, 30% B 0-5 min, 30-70% B 5-35 min, 99% B 35-45 min. The product was obtained as colourless oil after preparative HPLC and lyophilization. (53.4 mg, 40 μmol, 21%). HR-MS for C₅₇H₁₀₆NO₂₈P²⁺ [M+2H]²⁺ calcd.: 641.8314, found 641.84318.FIG. 249 shows an HPLC/UV chromatogram of the P5(PEG24)-COOH.

### General Method 3 for Nitrophenyl Phosphoramidates from 4-Nitrophenyl Phosphorodichloridate

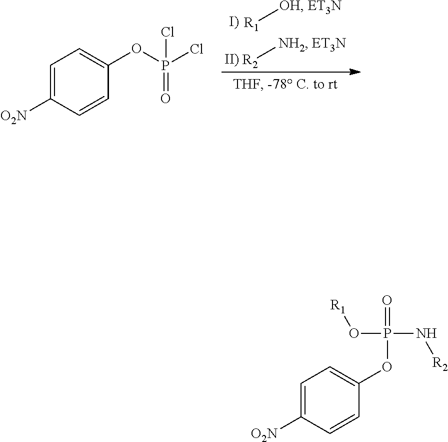

A solution of 4-nitrophenyl phosphorodichloridate (0.5 g, 1.95 mmol) in THF (6 mL) was cooled to −78° C. under argon atmosphere. To this clear solution, triethylamine (1.17 mL, 8.4 mmol) was added followed by dropwise addition of a suitable alcohol (1 eq., 1.95 mmol). The suspension was stirred at room temperature for 2 h. The reaction was again cooled to −78° C. and to this solution, a suitable amine, amino acid or peptide (0.354 g, 1.95 mmol) was added in one portion. The resulted solution was stirred at room temperature for 3 h. The reaction mixture was diluted with EtOAc (15 mL) and filtered through a Buchner funnel. The filtrate obtained was washed with water and brine. The organic layer was dried over Na2SO4, filtered and concentrated in vacuo to obtain the crude material which was purified by silica gel chromatography using a gradient elution (EtOAC: Cyclohexane; 0:100 to 25:70) to give the desired 4-nitrophenyl phophoramidate.

### General Method 4 for Boc Deprotection in the Presence of the Tert-Butyl Ester Followed by Amide Coupling

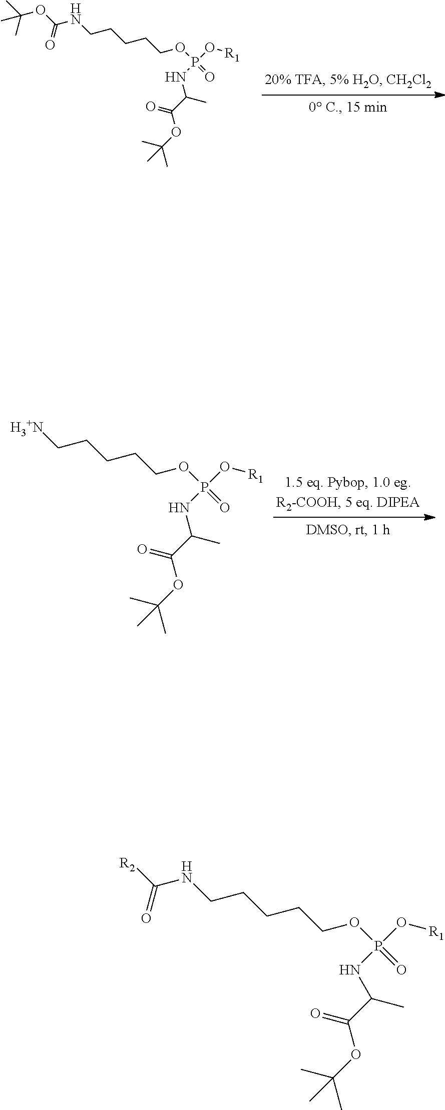

The Boc protected phosphoramidate was dissolved in 0.5 ml DCM and 5% H2O were added. After cooling the mixture to 0° C. in an ice bath, 0.2 ml TFA were added and the solution was stirred at 0° C. for 30 min to 3 h until deprotection was complete. The solvents were removed under N₂ stream and the product was dried in vacuo. The products were directly transferred to the next step without any further purification.

The residue was dissolved in 0.5 ml DMSO. 1.5 eq. PyBop, dissolved in DMSO and 5.0 eq. DIPEA were added. After 5 minutes, 1.0 eq of desired carboxylic acid dissolved in DMSO was added and the mixture was stirred at RT for 1 h. The mixture was diluted with H2O/MeCN (2:1) and directly subjected to preparative HPLC purification.

### General Method 5 for Boc- and Tert-Butyl Ester Deprotection, Followed by Amide Coupling

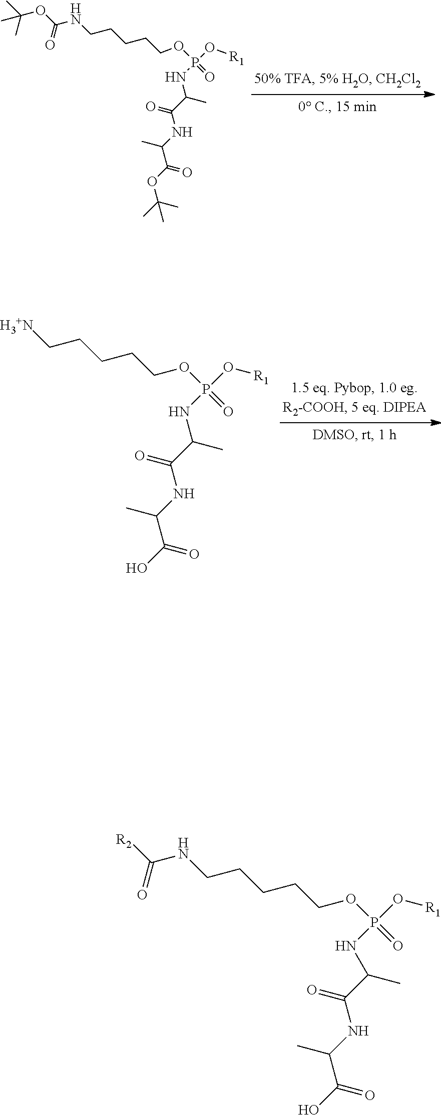

The Boc protected phosphoramidates were dissolved in 0.5 ml DCM and 5% H2O were added. After cooling the mixture to 0° C. in an ice bath, 0.5 ml TFA were added and the solution was stirred at 0° C. for 30 min to 3 h until deprotection was complete. The solvents were removed under N₂ stream and the product was dried in vacuo. The products were directly transferred to the next step without any further purification.

The residue was dissolved in 0.5 ml DMSO. 1.5 eq. PyBop, dissolved in DMSO and 5.0 eq. DIPEA were added. After 5 minutes, 1.0 eq of desired carboxylic acid dissolved in DMSO was added and the mixture was stirred at RT for 1 h. The mixture was diluted with H2O/MeCN (2:1) and directly subjected to preparative HPLC purification.

### General Method 6 for the Synthesis of Phosphoramidates from 4-Nitrophenol Phosphoramidates

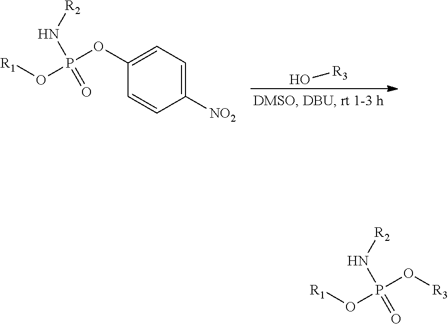

A respective alcohol is dissolved with 3 equivalents of a suitable 4-nitrophenol phosphoramidate precursor in DMSO. 6 equivalents DBU is added and the reaction is stirred at room temperature for 30 minutes to 3 hours, or until the alcohol is fully consumed. After completion, the reaction is diluted with H2O/MeCN (2:1) and directly subjected to preparative HPLC purification.

### General Method 7 for the Synthesis of Phosphoramidates from 4-Nitrophenol Phosphoramidates

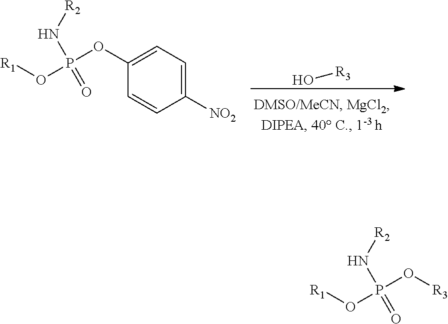

3 equivalents magnesium chloride were placed in a Schlenk flask equipped with magnetic stirrer and heat dried under vacuum. A respective alcohol is dissolved with 3 equivalents of a suitable 4-nitrophenol phosphoramidate precursor in DMSO and added to the magnesium chloride under argon. 3 equivalents DIPEA is added and the reaction is heated to 40° C. and stirred for 30 minutes to 3 hours, or until the alcohol is fully consumed. After completion, the reaction is diluted with H2O/MeCN (2:1) and directly subjected to preparative HPLC purification.

### General Method 8 for the Synthesis of Phosphoramidates from Boc-Aminopentane Phenyl Phosphite, Alcohols and Amines

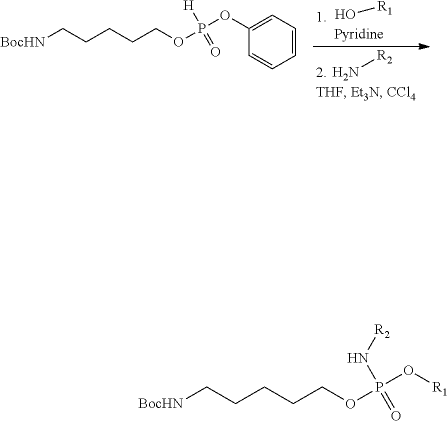

10 equivalents boc-aminopentane phenyl phosphite were dissolved in 0.5 mL dry pyridine/100 mg and transferred to a heat dried and argon flushed 25 mL Schlenk flask equipped with magnetic stir bar and septum. 1 equivalent of alcohol dissolved in 0.5 mL pyridine/10 mg were added to the phosphite solution and stirred under an argon atmosphere for 1 h at room temperature or until the alcohol is fully consumed. Pyridine was evaporated and the crude residue is dissolved in dry THF. 20 equivalents diAlaOtBu were dissolved in dry THF and transferred to a heat-dried and argon flushed 25 mL Schlenk flask equipped with magnetic stir bar and septum. The phosphite in THF was added to the stirring solution of diAlaOtBu and stirred at room temperature under argon. Subsequently, 20 equivalents of anhydrous triethylamine and 40 equivalents of carbon tetrachloride were added. The reaction was stirred at room temperature for 2 h or until all phosphite was consumed. The solvents were evaporated in an argon stream and the crude residue was purified via HPLC to yield the desired phosphoramidate.

### Synthesis of boc-aminopentane phenyl phosphite

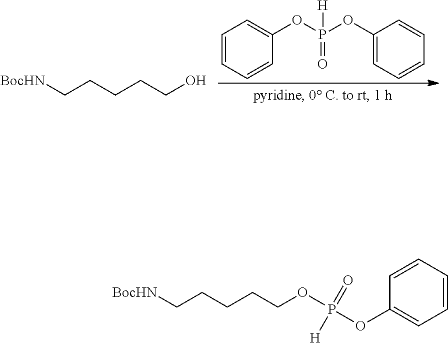

A 25 mL Schlenk-flask, equipped with magnetic stir bar and septum, was heat dried under vacuum and flushed with argon. The Schlenk-flask was charged with 1.7 g diphenyl phosphite (7.4 mmol, 3 equivalents) and dissolved in 5 mL dry pyridine. The phosphite solution was cooled to 0° C. with wet ice and 0.5 g boc-aminopentanol (2.5 mmol, 1 equivalent) dissolved in 3 mL dry pyridine were added dropwise. After complete addition, the wet ice was removed and the reaction was stirred for 60 minutes under argon. Pyridine was evaporated under reduced pressure and the residue was suspended in cyclohexane/EtOAc (95:5) and purified by flash column chromatography (cyclohexane/EtOAc, 0-80% EtOAc, 5 CV 0% EtoAc, 10 CV to 50% EtOAc and 10 CV to 80% EtOAc). 0.53 g of boc-aminopentan phenyl phosphite (1.5 mmol, 62.4%) were isolated as clear colourless oil. HR-MS for C₁₆ H₂₇NO₅P⁺ [M+H]⁺ calcd.: 344.1621, found: 344.16277.

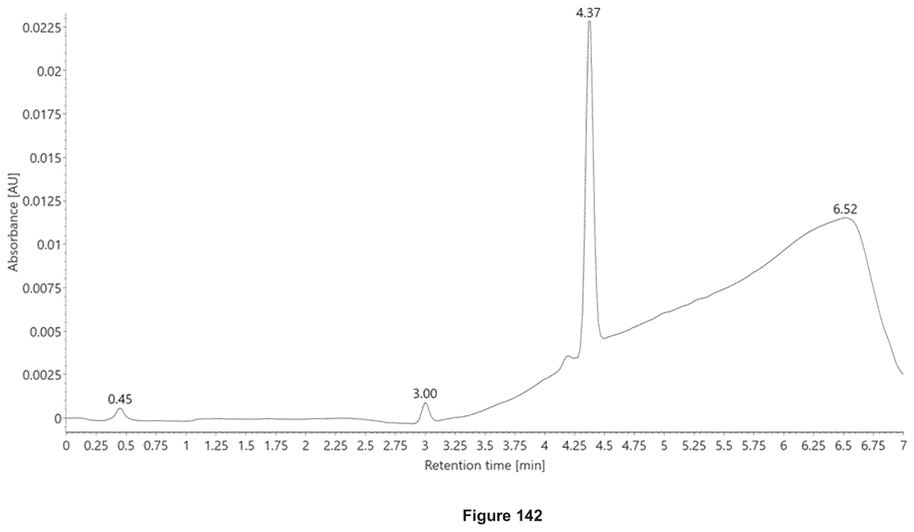

FIG. 142 shows an HPLC/UV chromatogram of the compound boc-aminopentane phenyl phosphite.

### O-(5-tert.-butoxy-carbonyl)-amidopentyl-Phosphoramidate-N-(L-alanine-iso-propylester)-O-4-nitrophenyl

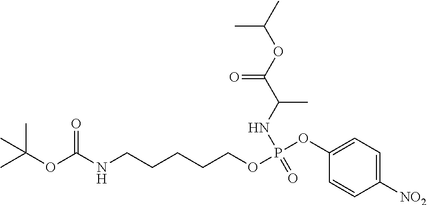 [SMILES: CC(C)OC(=O)C(C)NP(=O)(OCCCCCNC(=O)OC(C)(C)C)Oc1ccc([N+](=O)[O-])cc1]

The title compound was synthesized in accordance with the general Method 3 for the synthesis of nitrophenyl phosphoramidates from 269 mg 4-nitrophenyl phosphorodichloridate (1.05 mmol), 203 mg Boc-aminopentanol (1.0 mmol) and 184 mg L-alanine isopropyl ester hydrochloride (1.1 mmol). The product was obtained as white solid (367 mg, 0.71 mmol, 71%). MS for C₂₂H₃₇N₃O₉P⁺ [M+H]⁺ calcd.: 518.2 found 518.3.

### O-(5-tert.-butoxy-carbonyl)-aminopentvl-Phosphoramidate-N-(L-alanine-iso-propvlester)-O-SN38

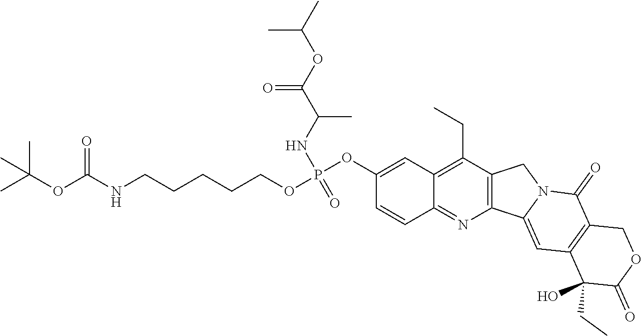 [SMILES: CCc1c2c(nc3ccc(OP(=O)(NC(C)C(=O)OC(C)C)OCCCCCNC(=O)OC(C)(C)C)cc13)-c1cc3c(c(=O)n1C2)COC(=O)[C@]3(O)CC]

The title compound was synthesized in accordance with general Method 6 from 6 mg SN38 (0.016 mmol) and 16 mg O-(5-tert.-butoxy-carbonyl-amidopentyl)-Phosphoramidate-N-(L-alanine-iso-propylester)-O-4-nitrophenyl (0.031 mmol). The product was obtained as white solid after preparative HPLC (Method C) and lyophilization. (8.5 mg, 0.011 mmol, 69%). HR-MS for C₃₈H₅₂N₄O₁₁P⁺ [M+H]⁺ calcd.: 771.3365 found 771.3374.

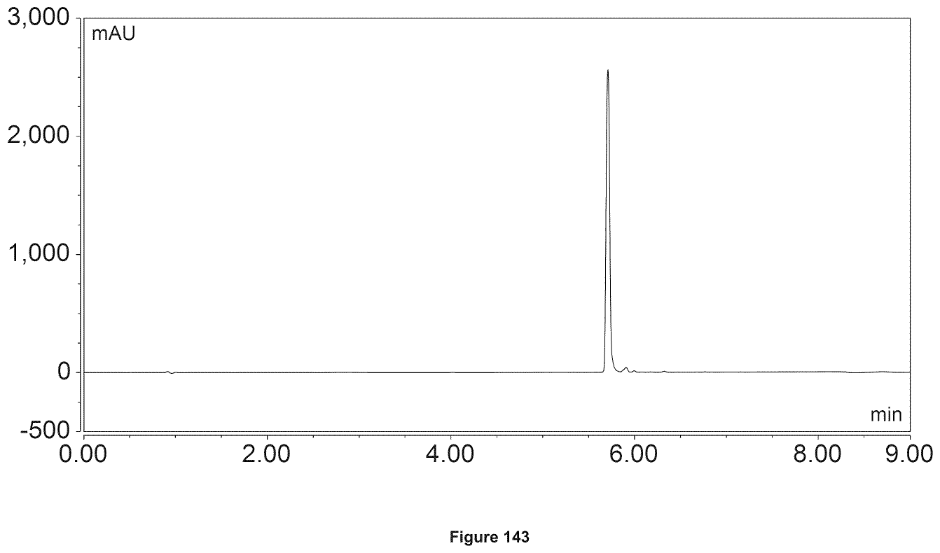

FIG. 143 shows an HPLC/UV chromatogram of the compound O-(5-tert.-butoxy-carbonyl)-aminopentyl-Phosphoramidate-N-(L-alanine-iso-propylester)-O—SN38.

### O—P5(PEG24)-amidopentyl-Phosphoramidate-N-(L-alanine-iso-propylester)-O—SN38

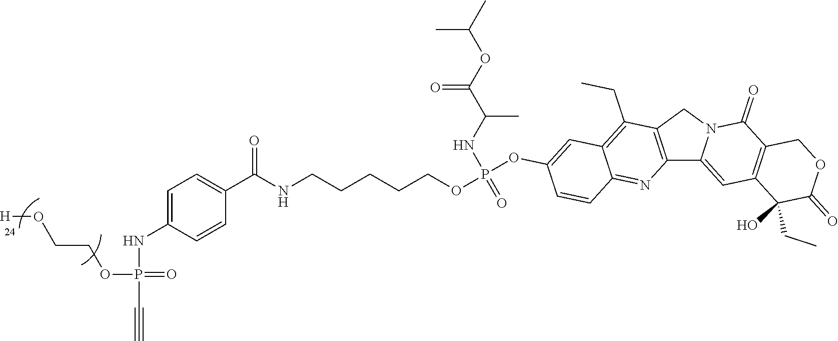

The title compound was synthesized in accordance to general method for Boc-deprotection followed by amide coupling from 8.5 mg of O-(5-tert.-butoxy-carbonyl-aminopentyl)-Phosphoramidate-N-(L-alanine-iso-propylester)-O—SN38 (0.011 mmol, 1 eq). The product was obtained as yellowish oil after preparative HPLC (Method C) and lyophilization. (16.7 mg, 0.009 mmol, 81% over two steps). HR-MS for C₉₀H₁₄₇N₅O₃₆P₂²⁺ [M+2H]⁺ calcd.: 967.9645, found 967.9616.

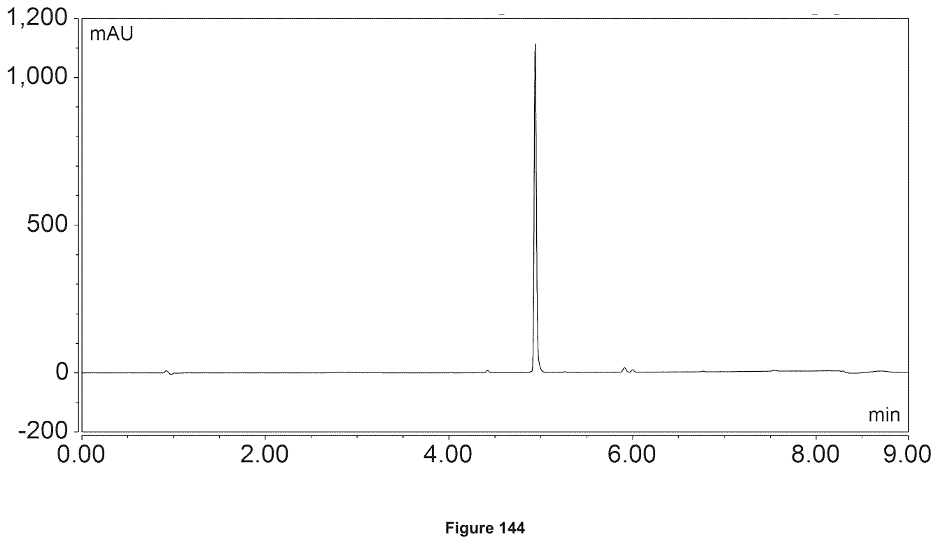

FIG. 144 shows an HPLC/UV chromatogram of the compound O—P5(PEG24)-amidopentyl-Phosphoramidate-N-(L-alanine-iso-propylester)-O—SN38.

### O-(5-tert.-butoxy-carbonyl)-aminopentvl-Phosphoramidate-N-(L-alanine-iso-propvlester)-O—ON-013100

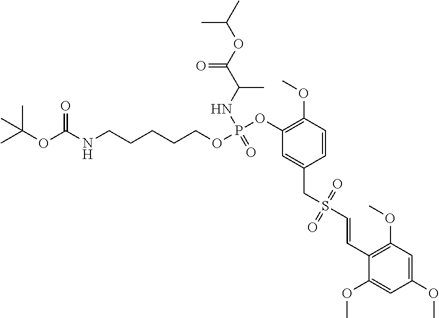 [SMILES: COc1cc(OC)c(/C=C/S(=O)(=O)Cc2ccc(OC)c(OP(=O)(NC(C)C(=O)OC(C)C)OCCCCCNC(=O)OC(C)(C)C)c2)c(OC)c1]

The title compound was synthesized in accordance with general Method 1 from 6.0 mg ON-013100 (0.015 mmol) and 24 mg O-(5-tert.-butoxy-carbonyl-amidopentyl)-Phosphoramidate-N-(L-alanine-iso-propylester)-O-4-nitrophenyl (0.046 mmol). The product was obtained as yellow solid after preparative HPLC (Method C) and lyophilization. (8.4 mg, 0.011 mmol, 73%). HR-MS for C₃₅H₅₄N₂O₁₃PS⁺ [M+H]⁺ calcd.: 773.3079 found 773.3118.

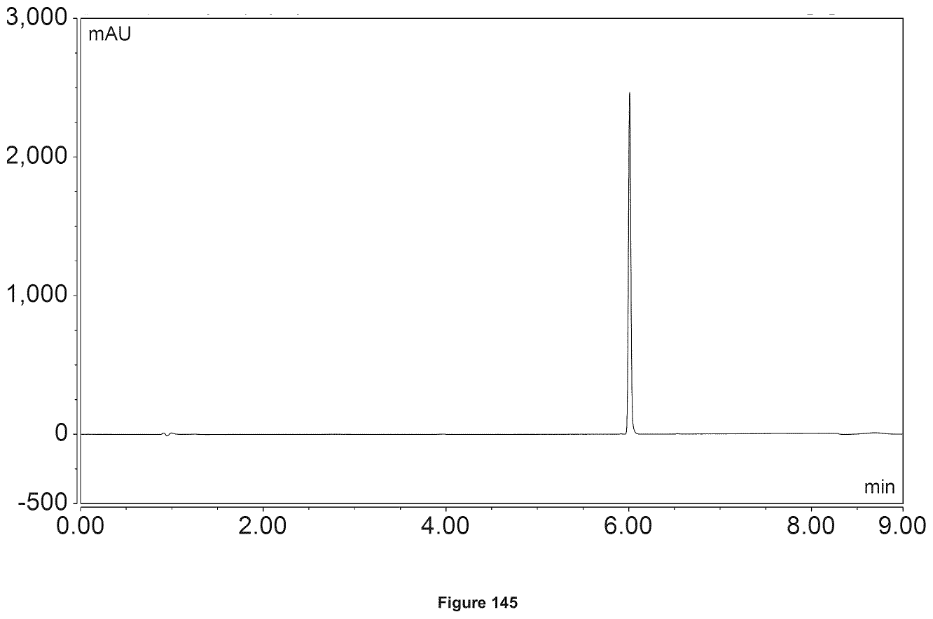

FIG. 145 shows an HPLC/UV chromatogram of the compound O-(5-tert.-butoxy-carbonyl)-aminopentyl-Phosphoramidate-N-(L-alanine-iso-propylester)-O—ON-013100.

### O—P5(PEG24)-amidopentyl-Phosphoramidate-N-(L-alanine-iso-propvlester)-O—ON-013100

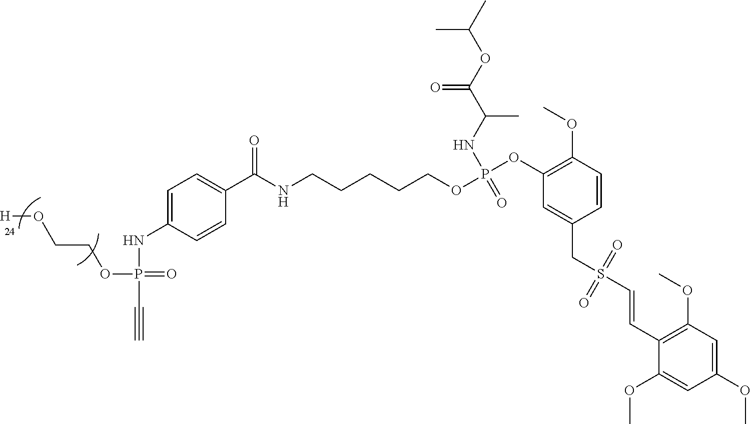

The title compound was synthesized in accordance to general method for Boc- and tert-butyl deprotection followed by amide coupling from 8.4 mg of O-(5-tert.-butoxy-carbonyl-aminopentyl)-Phosphoramidate-N-(L-alanine-iso-propylester)-O—ON-013100 (0.011 mmol, 1 eq). The product was obtained as yellowish oil after preparative HPLC (Method C) and lyophilization. (11.7 mg, 0.006 mmol, 55% over two steps). HR-MS for C₈₇H₁₄₉N₃O₃₈P₂S²⁺[M+2H]²⁺ calcd.: 968.9502, found 968.9505.

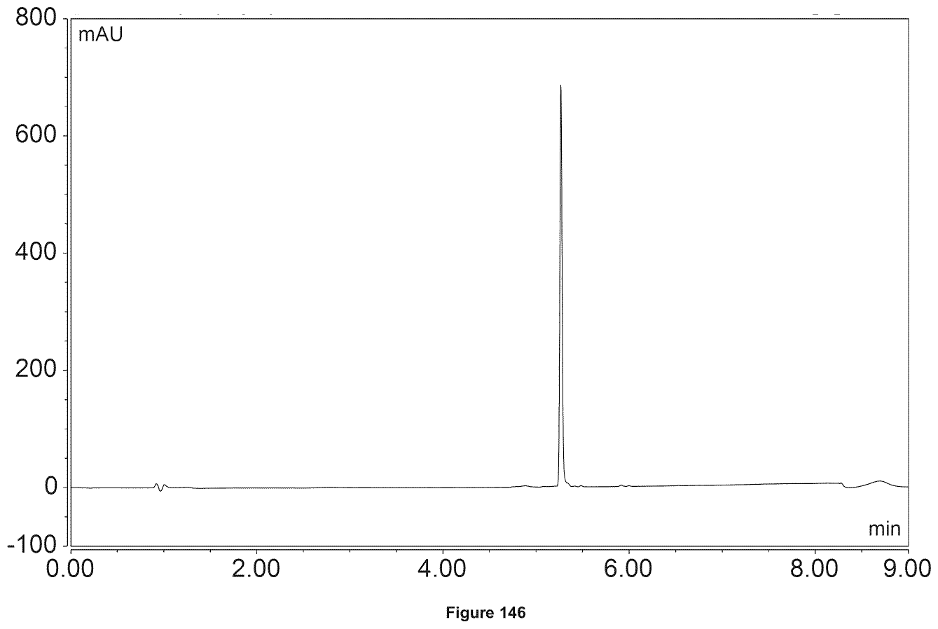

FIG. 146 shows an HPLC/UV chromatogram of the compound O—P5(PEG24)-amidopentyl-Phosphoramidate-N-(L-alanine-iso-propylester)-O—ON-013100.

### O-(5-tert.-butoxy-carbonyl)-aminopentvl-Phosphoramidate-N-(L-alanine-iso-propvlester)-O— Ganetespib

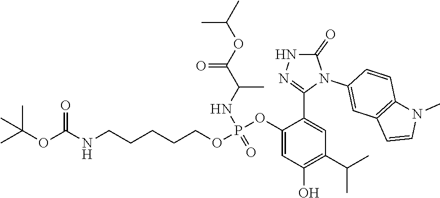 [SMILES: CC(C)OC(=O)C(C)NP(=O)(OCCCCCNC(=O)OC(C)(C)C)Oc1cc(O)c(C(C)C)cc1-c1n[nH]c(=O)n1-c1ccc2c(ccn2C)c1]

The title compound was synthesized in accordance with general Method 1 from 7.0 mg Ganetespib (0.019 mmol) and 10 mg O-(5-tert.-butoxy-carbonyl-amidopentyl)-Phosphoramidate-N-(L-alanine-iso-propylester)-O-4-nitrophenyl (0.019 mmol). The product was obtained as white solid after preparative HPLC (Method C) and lyophilization. (1.6 mg, 0.002 mmol, 11%). HR-MS for C₃₆H₅₂N₅O₉P⁺ [M+H]⁺ calcd.: 743.3528 found 743.3542.

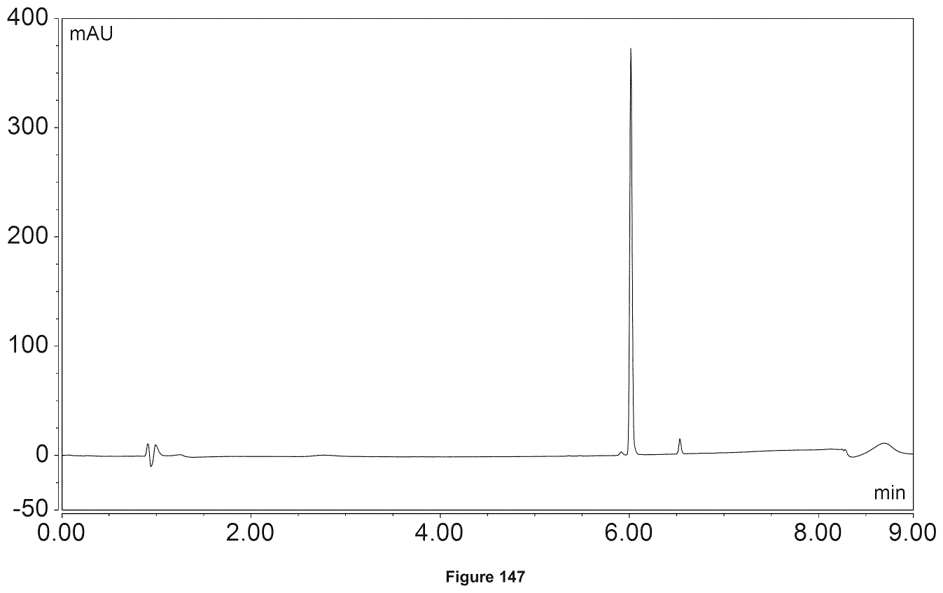

FIG. 147 shows an HPLC/UV chromatogram of the compound O-(5-tert.-butoxy-carbonyl)-aminopentyl-Phosphoramidate-N-(L-alanine-iso-propylester)-O-Ganetespib.

### O—P5(PEG24)-amidopentyl-Phosphoramidate-N-(L-alanine-iso-propvlester)-O-Ganetespib

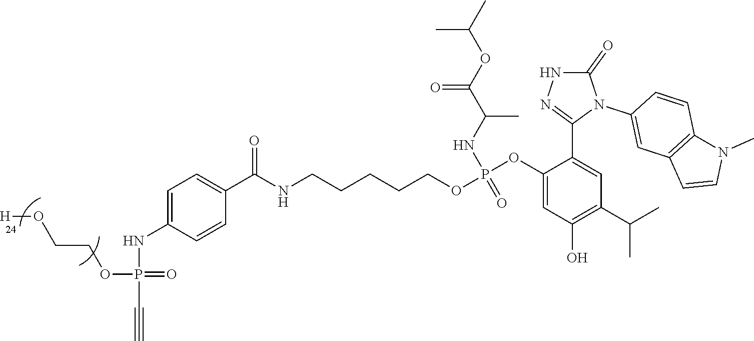

The title compound was synthesized in accordance to general method for Boc- and tert-butyl deprotection followed by amide coupling from 1.6 mg of O-(5-tert.-butoxy-carbonyl-aminopentyl)-Phosphoramidate-N-(L-alanine-iso-propylester)-O-Ganetespib (0.002 mmol, 1 eq). The product was obtained as yellowish oil after preparative HPLC (Method C) and lyophilization. (2.6 mg, 0.001 mmol, 65% over two steps). HR-MS for C₈₈H₁₄₇N₇O₃₄P₂²⁺ [M+2H]²⁺ calcd.: 953.9727, found 953.9735.

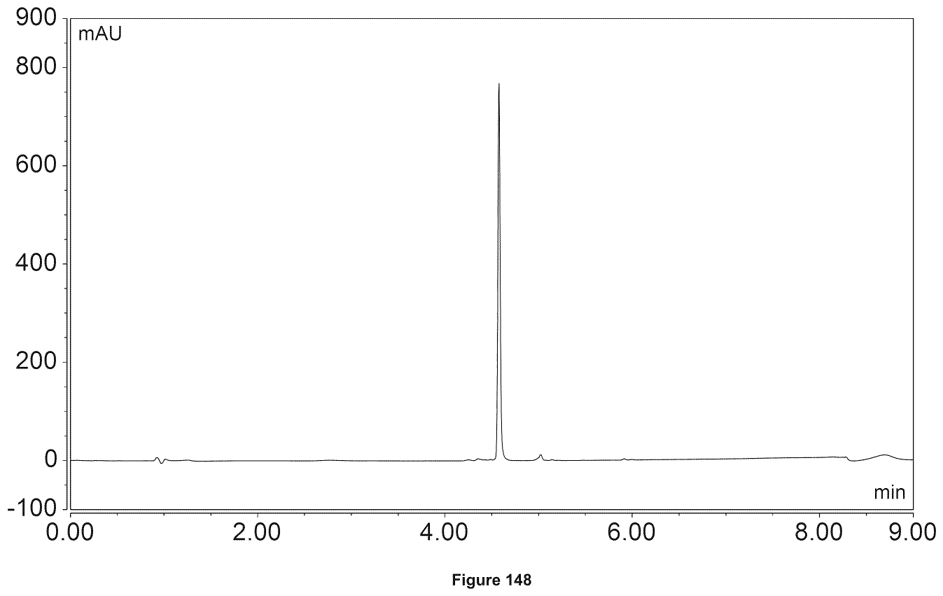

FIG. 148 shows an HPLC/UV chromatogram of the compound O—P5(PEG24)-amidopentyl-Phosphoramidate-N-(L-alanine-iso-propylester)-O-Ganetespib.

### O-(5-tert.-butoxy-carbonyl)-amidopentyl-Phosphoramidate-N-(plycine-tert.-butylester)-O-4-nitrophenyl

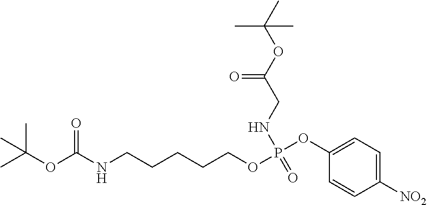 [SMILES: CC(C)(C)OC(=O)CNP(=O)(OCCCCCNC(=O)OC(C)(C)C)Oc1ccc([N+](=O)[O-])cc1]

The title compound was synthesized in accordance with the general method for the synthesis of nitrophenyl phosphoramidates from 269 mg 4-nitrophenyl dichlorophosphate (1.05 mmol), 203 mg Boc-aminopentanol (1.0 mmol) and 184 mg glycine tert.-butyl ester hydrochloride (1.1 mmol). The product was obtained as white solid (393 mg, 0.76 mmol, 76%). MS for C₂₂H₃₇N₃O₉P⁺ [M+H]⁺ calcd.: 518.2 found 518.3.

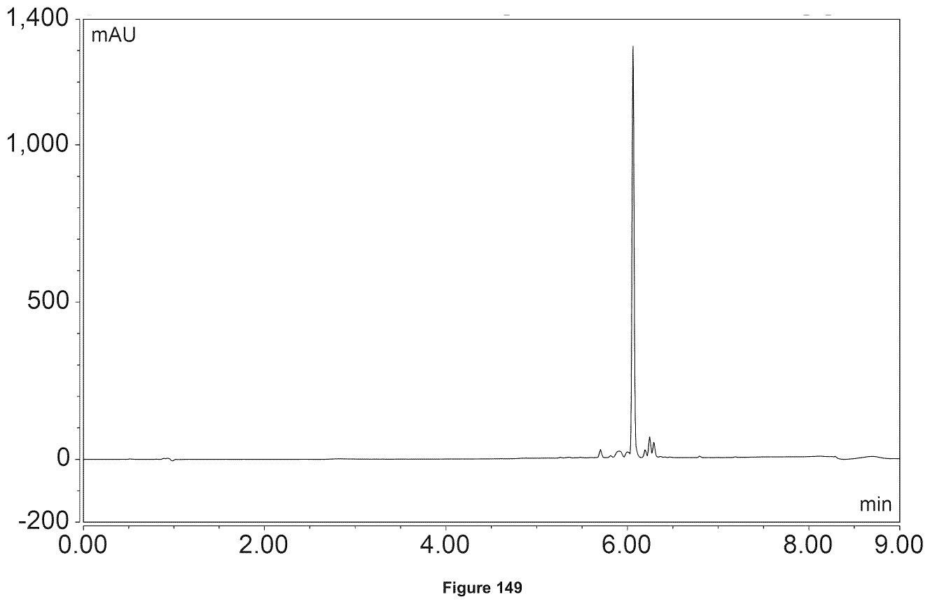

FIG. 149 shows an HPLC/UV chromatogram of the compound O-(5-tert.-butoxy-carbonyl)-amidopentyl-Phosphoramidate-N-(glycine-tert.-butylester)-O-4-nitrophenyl.

### O-(5-tert.-butoxy-carbonyl)-amidopentyl-Phosphoramidate-N-(glycine-tert.-butylester)-O-SN38

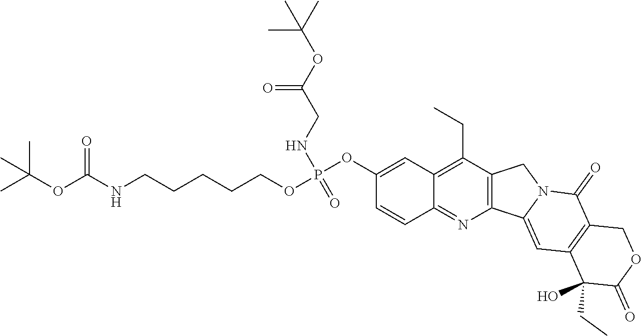 [SMILES: CCc1c2c(nc3ccc(OP(=O)(NCC(=O)OC(C)(C)C)OCCCCCNC(=O)OC(C)(C)C)cc13)-c1cc3c(c(=O)n1C2)COC(=O)[C@]3(O)CC]

The title compound was synthesized in accordance with general Method 1 from 6.6 mg SN38 (0.017 mmol) and 26 mg O-(5-tert.-butoxy-carbonyl-amidopentyl)-Phosphoramidate-N-(glycine-tert.-butylester)-O-4-nitrophenyl (0.05 mmol). The product was obtained as white solid after preparative HPLC (Method C) and lyophilization. (11 mg, 0.014 mmol, 82%). HR-MS for C₃₈H₅₂N₄O₁₁P⁺ [M+H]⁺ calcd.: 771.3365 found 771.3374.

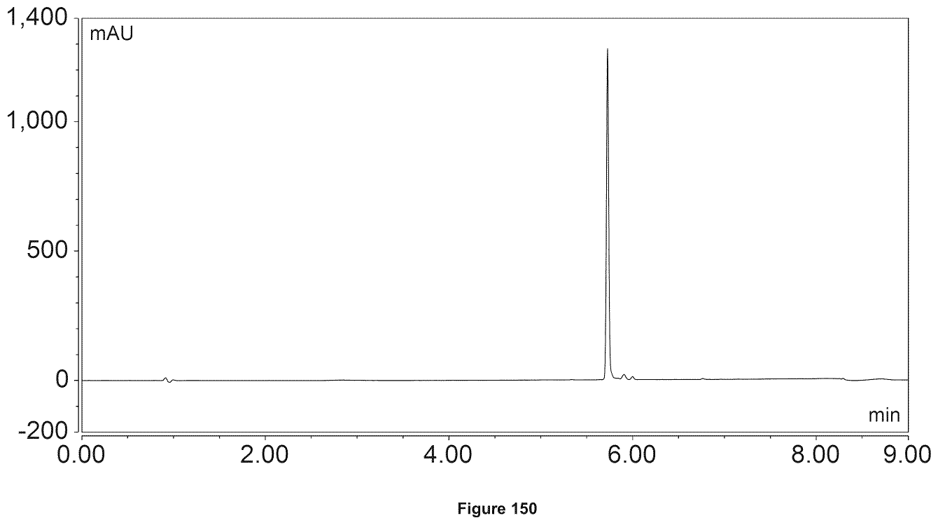

FIG. 150 shows an HPLC/UV chromatogram of the compound O-(5-tert.-butoxy-carbonyl)-amidopentyl-Phosphoramidate-N-(glycine-tert.-butylester)-O—SN38.

### O—P5(PEG24)-amidopentyl-Phosphoramidate-N-(L-alanine-iso-propylester)-O—SN38

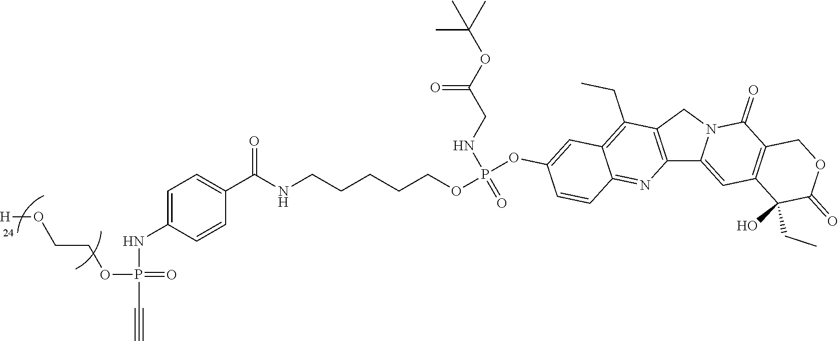

The title compound was synthesized in accordance to general Method 4 for Boc deprotection in the presence of the tert-butyl ester followed by amide coupling from 11 mg of O-(5-tert.-butoxy-carbonyl-amidopentyl)-Phosphoramidate-N-(glycine-tert.-butylester)-O—SN38 (0.011 mmol, 1 eq). The product was obtained as yellowish oil after preparative HPLC (Method C) and lyophilization. (16.7 mg, 0.009 mmol, 81% over two steps). HR-MS for C₉₀H₁₄₇N₅O₃₆P₂²⁺ [M+2H]²⁺ calcd.: 967.9645, found 967.9616.

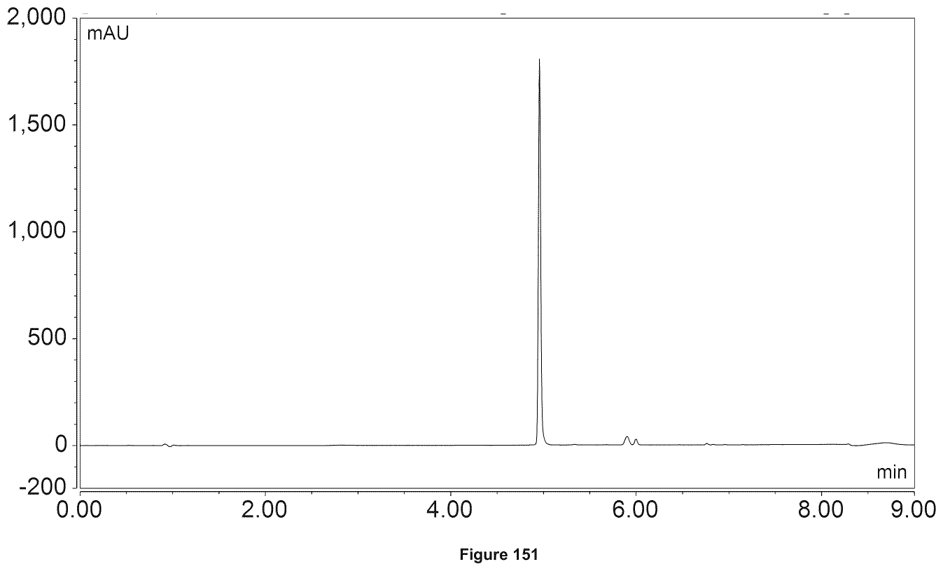

FIG. 151 shows an HPLC/UV chromatogram of the compound O—P5(PEG24)-amidopentyl-Phosphoramidate-N-(L-alanine-iso-propylester)-O—SN38.

### O-(5-tert.-butoxy-carbonyl)-amidopentyl-Phosphoramidate-N-(2,2-dimethylaminobutyric acid-tert.-butylester)-O-4-nitrophenyl

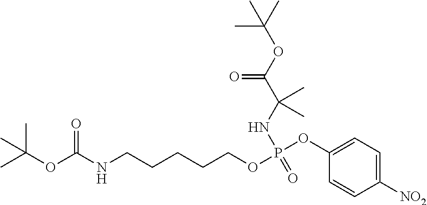 [SMILES: CC(C)(C)OC(=O)NCCCCCOP(=O)(NC(C)(C)C(=O)OC(C)(C)C)Oc1ccc([N+](=O)[O-])cc1]

The title compound was synthesized in accordance with the general Method for the synthesis of nitrophenyl phosphoramidates from 269 mg 4-nitrophenyl dichlorophosphate (1.05 mmol), 203 mg Boc-aminopentanol (1.0 mmol) and 175 mg 2,2-dimethylaminobutyric acid tert-butylester (1.1 mmol). The product was obtained as white solid (327 mg, 0.6 mmol, 60%). MS for C₂₄H₄₁N₃O₉P⁺ [M+H]⁺ calcd.: 546.3, found 546.4.

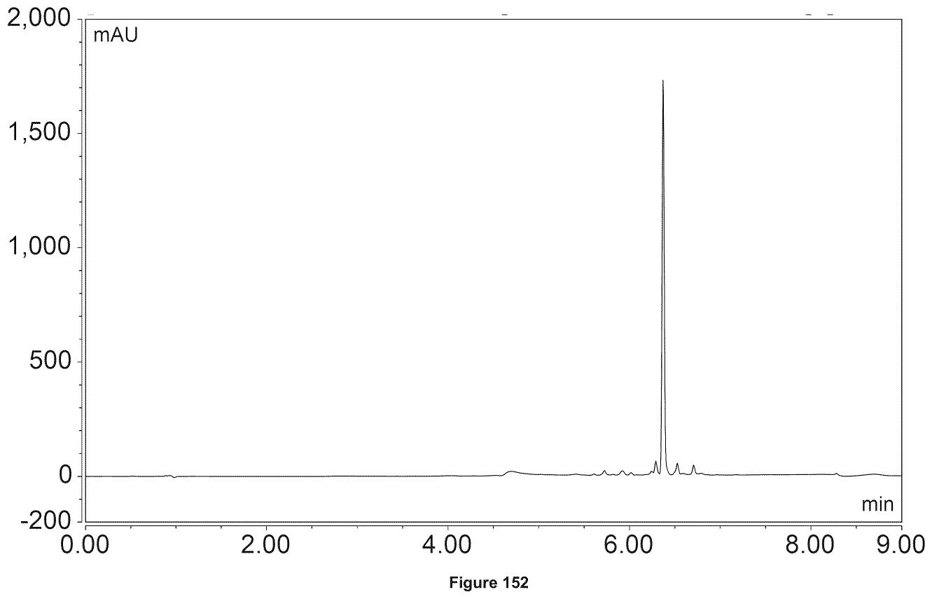

FIG. 152 shows an HPLC/UV chromatogram of the compound O-(5-tert.-butoxy-carbonyl)-amidopentyl-Phosphoramidate-N-(2,2-dimethylaminobutyric acid-tert.-butylester)-O-4-nitrophenyl.

### O-(5-tert.-butoxy-carbonyl)-amidopentyl-Phosphoramidate-N-(2,2-dimethylaminobutyric acid-tert.-butylester)-O-SN38

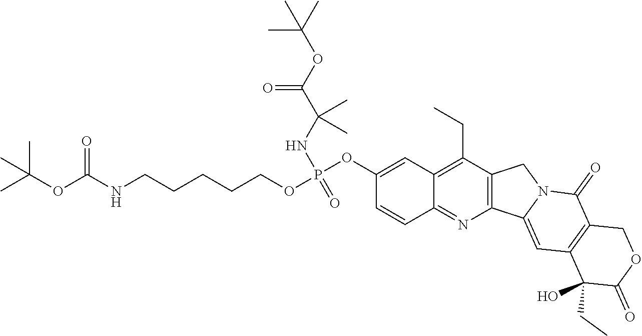 [SMILES: CCc1c2c(nc3ccc(OP(=O)(NC(C)(C)C(=O)OC(C)(C)C)OCCCCCNC(=O)OC(C)(C)C)cc13)-c1cc3c(c(=O)n1C2)COC(=O)[C@]3(O)CC]

The title compound was synthesized in accordance with general Method 6 from 6.1 mg SN38 (0.016 mmol) and 30 mg O-(5-tert.-butoxy-carbonyl-amidopentyl)-Phosphoramidate-N-(2,2-dimethylaminobutyric acid-tert.-butylester)-O-4-nitrophenyl (0.05 mmol). The product was obtained as white solid after preparative HPLC (Method C) and lyophilization. (9.3 mg, 0.012 mmol, 75%). HR-MS for C₄₀H₅₆N₄O₁₁P⁺ [M+H]⁺ calcd.: 799.3678 found 799.3690.

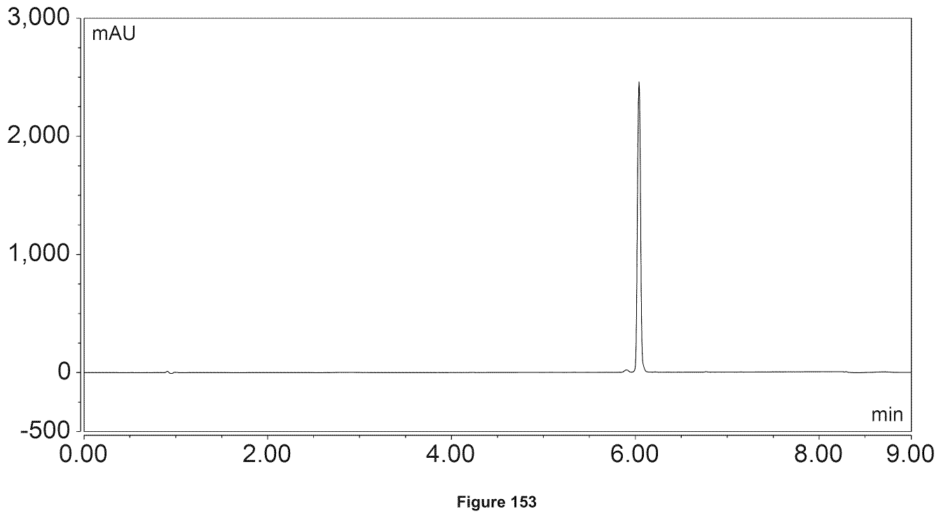

FIG. 153 shows an HPLC/UV chromatogram of the compound O-(5-tert.-butoxy-carbonyl)-amidopentyl-Phosphoramidate-N-(2,2-dimethylaminobutyric acid-tert.-butylester)-O—SN38.

### O—P5(PEG24)-amidopentyl-Phosphoramidate-N-(2,2-dimethylaminobutyric acid-tert.-butylester)-O-SN38

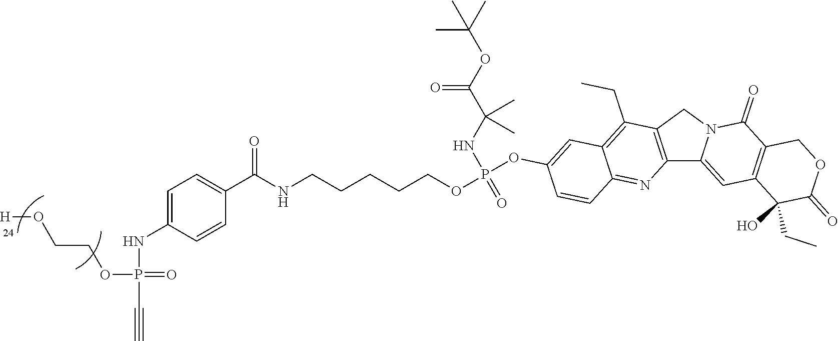

The title compound was synthesized in accordance to general Method 4 for Boc deprotection in the presence of the tert-butyl ester followed by amide coupling from 9.3 mg of O-(5-tert.-butoxy-carbonyl-amidopentyl)-Phosphoramidate-N-(2,2-dimethylaminobutyric acid-tert.-butylester)-O—SN38 (0.012 mmol, 1 eq). The product was obtained as yellowish oil after preparative HPLC (Method C) and lyophilization. (16.7 mg, 0.009 mmol, 81% over two steps). HR-MS for C₉₂H₁₅₁N₅O₃₆P₂²⁺ [M+2H]²⁺ calcd.: 981.9802, found 981.9803.

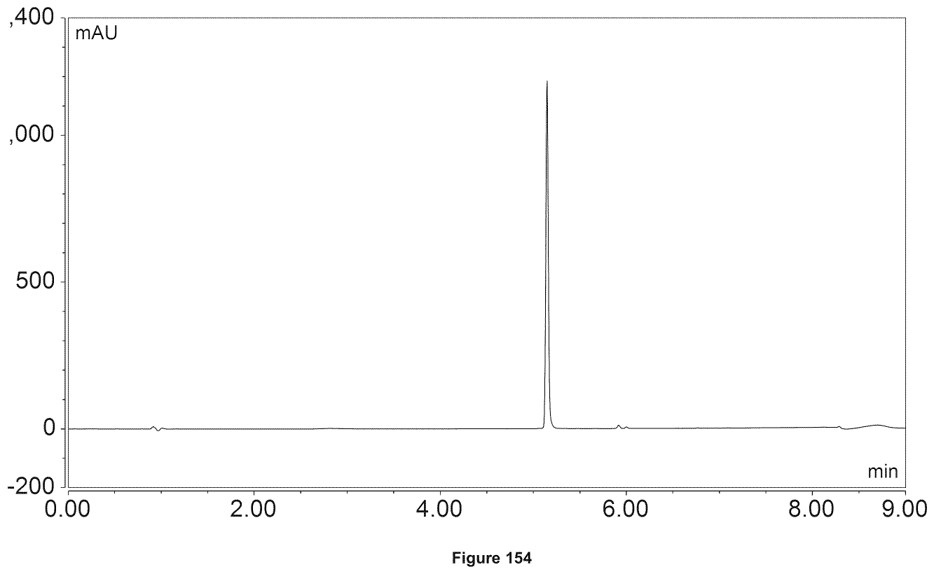

FIG. 154 shows an HPLC/UV chromatogram of the compound O—P5(PEG24)-amidopentyl-Phosphoramidate-N-(2,2-dimethylaminobutyric acid-tert.-butylester)-O—SN38.

### O-5-(5-tert.-butoxy-carbonyl)-amidopentyl-Phosphoramidate-N-(L-alanine-L-alanine-tert.-butylester)-O-SN38

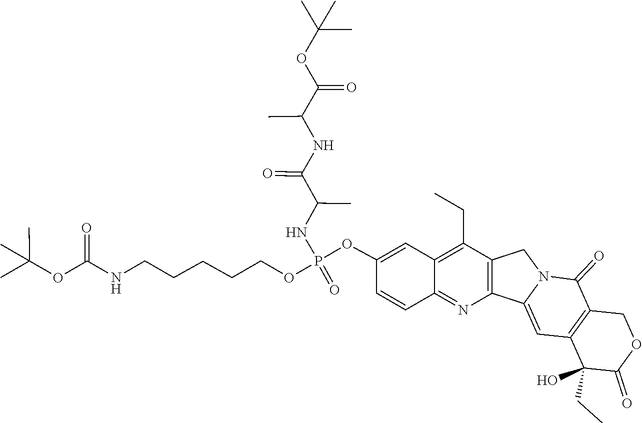 [SMILES: CCc1c2c(nc3ccc(OP(=O)(NC(C)C(=O)NC(C)C(=O)OC(C)(C)C)OCCCCCNC(=O)OC(C)(C)C)cc13)-c1cc3c(c(=O)n1C2)COC(=O)[C@]3(O)CC]

The title compound was synthesized in accordance with general Method 6 from 6.1 mg SN38 (0.016 mmol) and 25 mg O-(5-tert.-butoxy-carbonyl-amidopentyl)-Phosphoramidate-N-(L-alanine-L-alanine-tert.-butylester)-O-4-nitrophenyl (0.04 mmol). The product was obtained as yellow solid after preparative HPLC (Method C) and lyophilization. (4.6 mg, 0.005 mmol, 34%). HR-MS for C₄₂H₆₀N₅O₁₂P²⁺ [M+2H]²⁺ calcd.: 857.3965 found 857.3973.

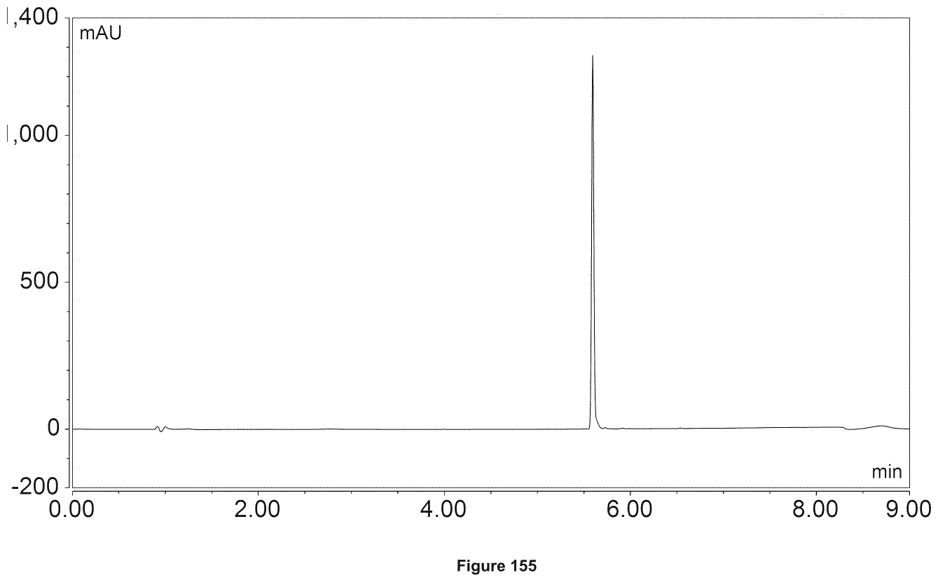

FIG. 155 shows an HPLC/UV chromatogram of the compound O-5-(5-tert.-butoxy-carbonyl)-amidopentyl-Phosphoramidate-N-(L-alanine-L-alanine-tert.-butylester)-O—SN38.

### O—P5(PEG24)-amidopentyl-Phosphoramidate-N-(L-alanine-L-alanine)-O—SN38

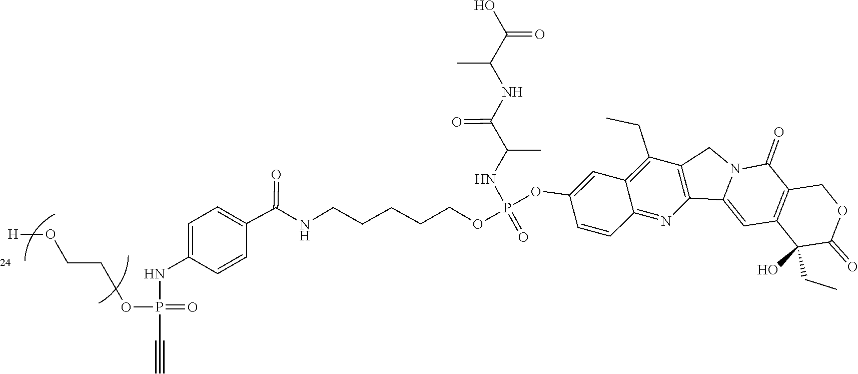

The title compound was synthesized in accordance to general Method 5 for Boc- and tert-butyl deprotection followed by amide coupling from 4.6 mg of O-(5-tert.-butoxy-carbonyl-amidopentyl)-Phosphoramidate-N-(L-alanine-L-alanine-tert.-butylester)-O—SN38 (0.005 mmol, 1 eq). The product was obtained as yellowish oil after preparative HPLC (Method C) and lyophilization. (3.8 mg, 0.002 mmol, 40% over two steps). HR-MS for C₉₀H₁₄₆FN₆O₃₇P₂²⁺ [M+2H]²⁺ calcd.: 982.4596, found 982.4569.

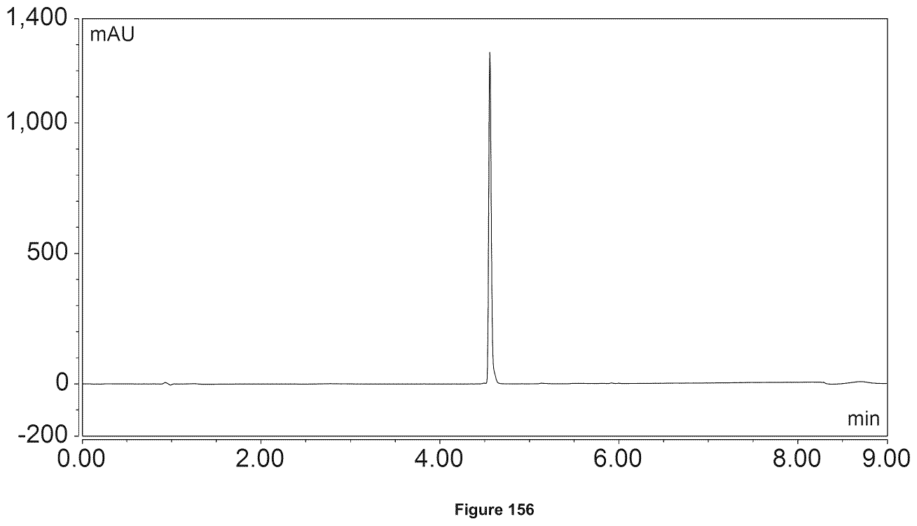

FIG. 156 shows an HPLC/UV chromatogram of the compound O—P5(PEG24)-amidopentyl-Phosphoramidate-N-(L-alanine-L-alanine)-O—SN38.

### O-5-tert.-butoxy-carbonyl-amidopentyl-Phosphoramidate-N-(L-alanine-L-alanine-tert.-butylester)-O—ON-013100

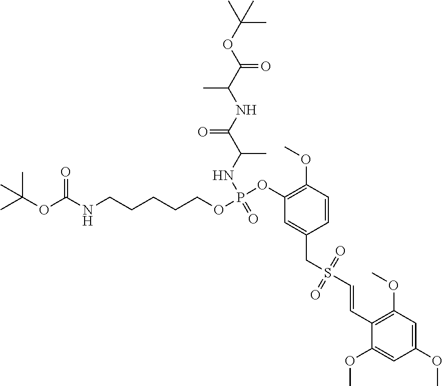 [SMILES: COc1cc(OC)c(/C=C/S(=O)(=O)Cc2ccc(OC)c(OP(=O)(NC(C)C(=O)NC(C)C(=O)OC(C)(C)C)OCCCCCNC(=O)OC(C)(C)C)c2)c(OC)c1]

The title compound was synthesized in accordance with general Method 6 from 6.0 mg ON-013100 (0.015 mmol) and 24 mg O-(5-tert.-butoxy-carbonyl-amidopentyl)-Phosphoramidate-N-(L-alanine-L-alanine-tert.-butylester)-O-4-nitrophenyl (0.046 mmol). The product was obtained as yellow solid after preparative HPLC (Method C) and lyophilization. (11.0 mg, 0.013 mmol, 87%). HR-MS for C₃₉H₆₁N₃O₁₄PS⁺ [M+H]⁺ calcd.: 858.3606 found 858.3623.

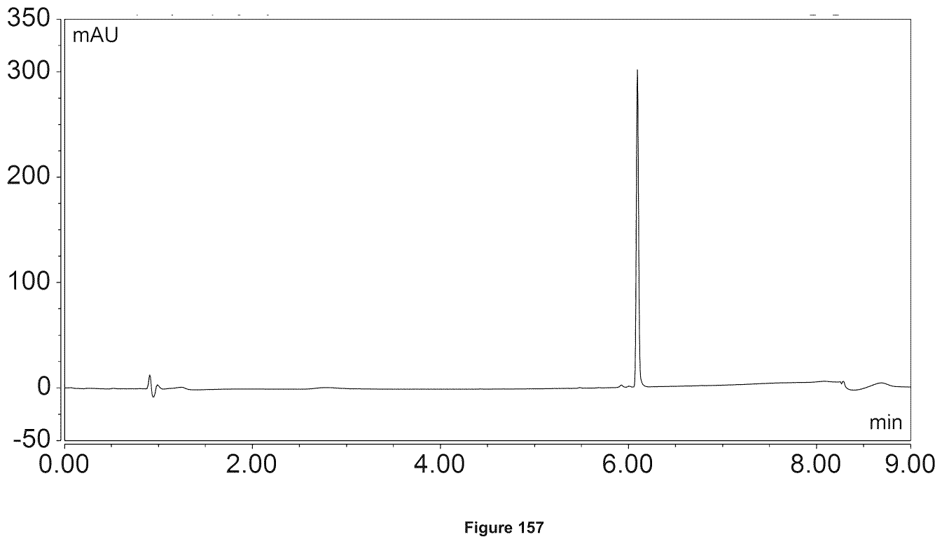

FIG. 157 shows an HPLC/UV chromatogram of the compound O-5-tert.-butoxy-carbonyl-amidopentyl-Phosphoramidate-N-(L-alanine-L-alanine-tert.-butylester)-O—ON-013100.

### O—P5(PEG24)-amidopentyl-Phosphoramidate-N-(L-alanine-L-alanine)-O—ON-013100

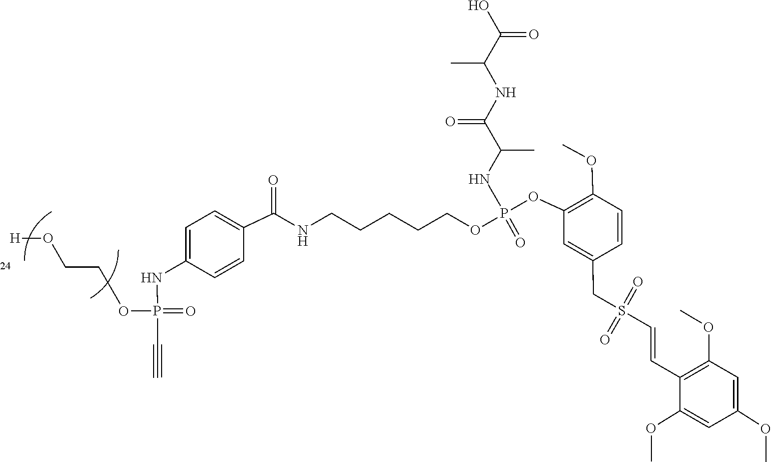

The title compound was synthesized in accordance to general Method 5 for Boc- and tert-butyl deprotection followed by amide coupling from 11 mg of O-(5-tert.-butoxy-carbonyl-amidopentyl)-Phosphoramidate-N-(L-alanine-L-alanine-tert.-butylester)-O—ON-013100 (0.013 mmol, 1 eq). The product was obtained as yellowish oil after preparative HPLC (Method C) and lyophilization. (3.2 mg, 0.002 mmol, 12% over two steps). HR-MS for C₈₇H₁₄₈N₄O₃₉P₂S²⁺[M+2H]²⁺ calcd.: 983.4453, found 983.4390.

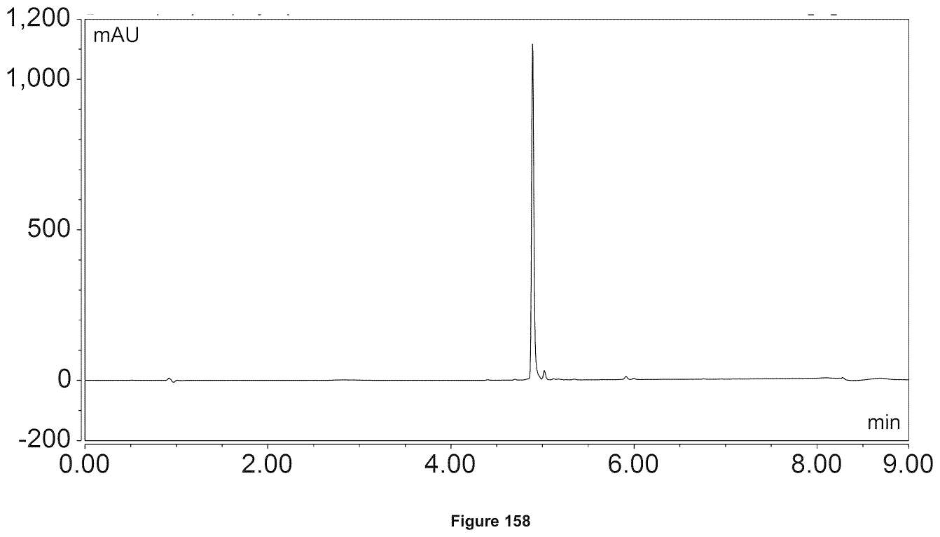

FIG. 158 shows an HPLC/UV chromatogram of the compound O—P5(PEG24)-amidopentyl-Phosphoramidate-N-(L-alanine-L-alanine)-O—ON-013100.

### O-5-(tert.-butoxy-carbonyl)-amidopentyl-Phosphoramidate-N-(L-alanine-L-alanine-tert.-butylester)-O-Ganetespib

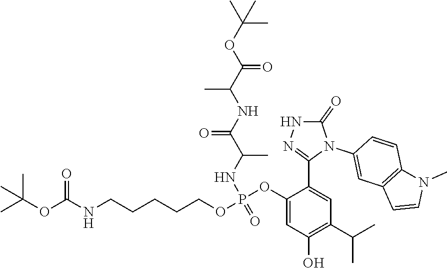 [SMILES: CC(NP(=O)(OCCCCCNC(=O)OC(C)(C)C)Oc1cc(O)c(C(C)C)cc1-c1n[nH]c(=O)n1-c1ccc2c(ccn2C)c1)C(=O)NC(C)C(=O)OC(C)(C)C]

The title compound was synthesized in accordance with general Method 6 from 10.0 mg Ganetespib (0.027 mmol) and 16.5 mg O-(5-tert.-butoxy-carbonyl-amidopentyl)-Phosphoramidate-N-(L-alanine-L-alanine-tert.-butylester)-O-4-nitrophenyl (0.027 mmol). The product was obtained as yellow solid after preparative HPLC (Method C) and lyophilization. (7.4 mg, 0.009 mmol, 33%). HR-MS for C₄₀H₅₉N₇O₁₀P⁺ [M+H]⁺ calcd.: 828.4056 found 828.4039.

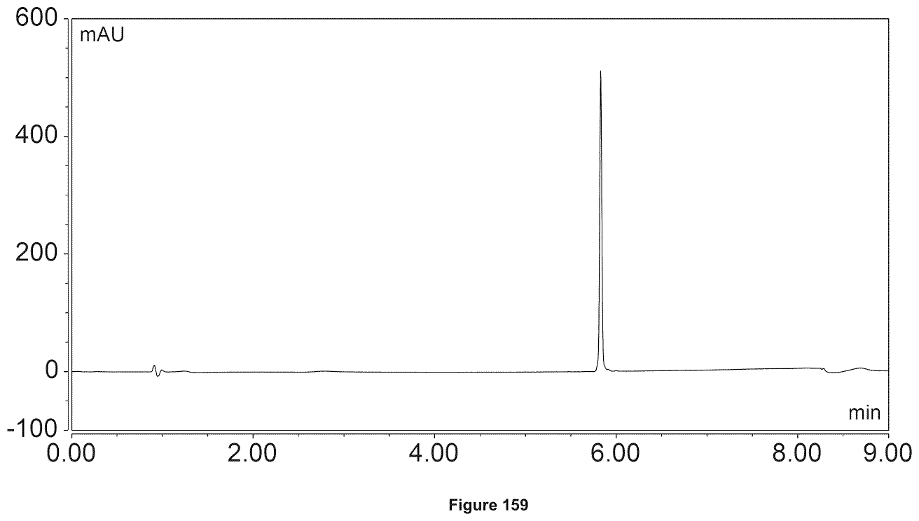

FIG. 159 shows an HPLC/UV chromatogram of the compound O-5-(tert.-butoxy-carbonyl)-amidopentyl-Phosphoramidate-N-(L-alanine-L-alanine-tert.-butylester)-O-Ganetespib.

### O—P5(PEG24)-amidopentyl-Phosphoramidate-N-(L-alanine-L-alanine)-O-Ganetespib

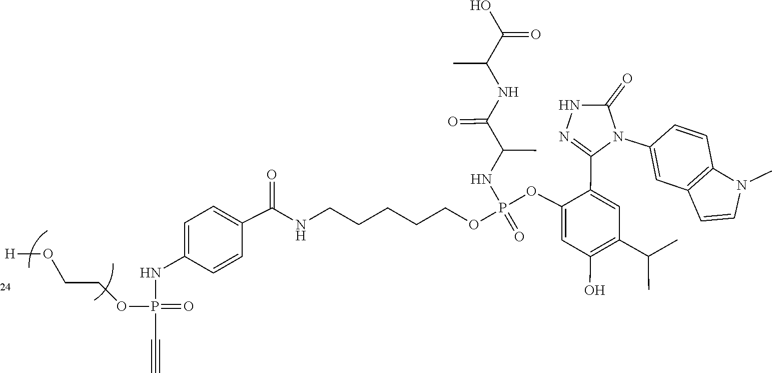

The title compound was synthesized in accordance to general Method 5 for Boc- and tert-butyl deprotection followed by amide coupling from 4 mg of O-(5-tert.-butoxy-carbonyl-amidopentyl)-Phosphoramidate-N-(L-alanine-L-alanine-tert.-butylester)-O— Ganetespib (0.005 mmol, 1 eq). The product was obtained as yellowish oil after preparative HPLC (Method C) and lyophilization. (1.2 mg, 0.6 μmol, 12% over two steps). HR-MS for C₈₈H₁₄₆N₈O₃₅P₂²⁺ [M+2H]²⁺ calcd.: 968.4678, found 968.4623.

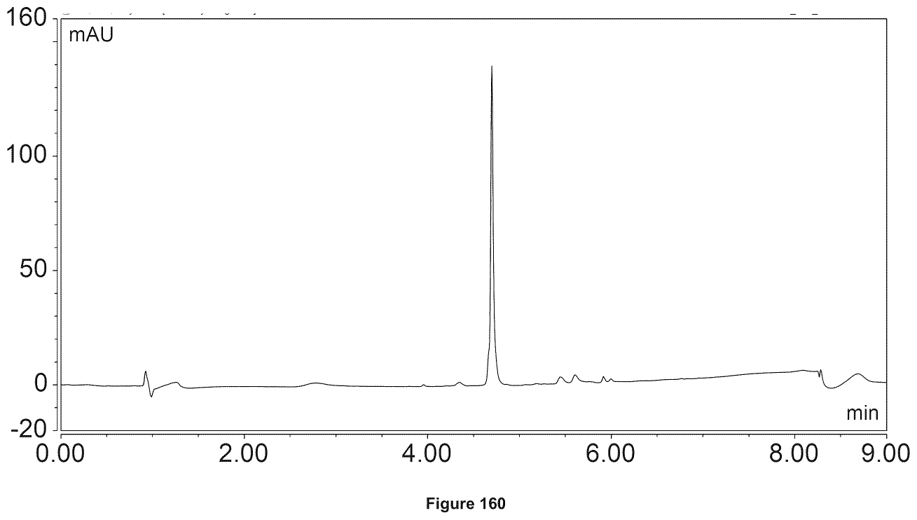

FIG. 160 shows an HPLC/UV chromatogram of the compound O—P5(PEG24)-amidopentyl-Phosphoramidate-N-(L-alanine-L-alanine)-O-Ganetespib.

### O-(5-tert.-butoxy-carbonyl)-amidocvclohexvl-Phosphoramidate-N-(L-alanine-L-alanine-tert.-butylester)-O-4-nitrophenyl

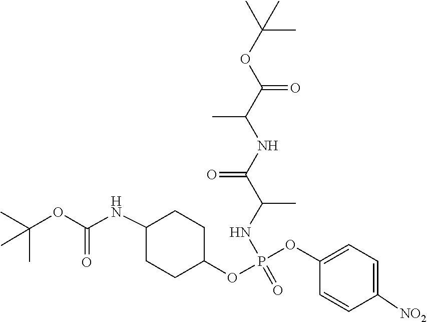 [SMILES: CC(NP(=O)(Oc1ccc([N+](=O)[O-])cc1)OC1CCC(NC(=O)OC(C)(C)C)CC1)C(=O)NC(C)C(=O)OC(C)(C)C]

The title compound was synthesized in accordance with the general Method 3 for nitrophenyl phosphoramidates from 269 mg 4-nitrophenyl phosphorodichloridate (1.05 mmol), 215 mg 4-(Boc-amino)cyclohexanol (1.0 mmol) and 330 mg L-alanine-L-alanine tert.-butyl ester trifluoroacetate (1.1 mmol). The product was obtained as white solid (412 mg, 0.67 mmol, 67%). MS for C₂₂H₃₇N₃O₉P⁺ [M+H]⁺ calcd.: 615.3 found, 615.3.

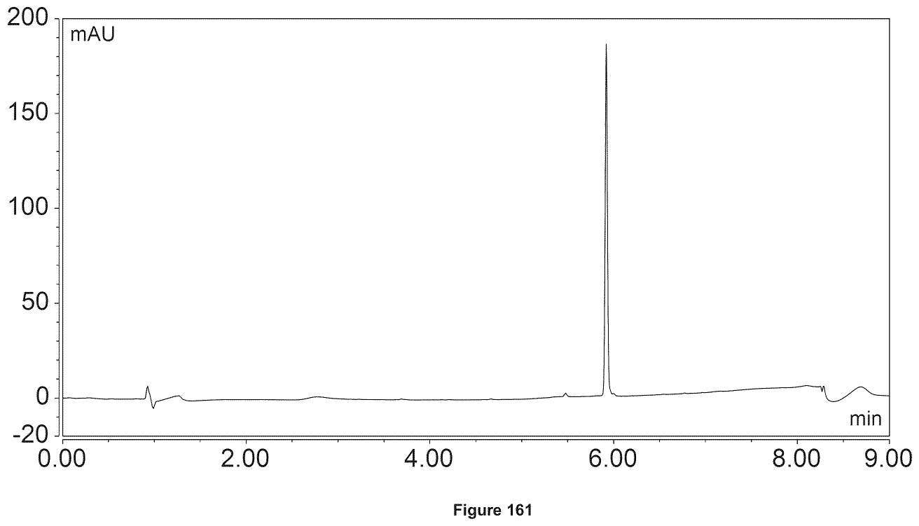

FIG. 161 shows an HPLC/UV chromatogram of the compound O-(5-tert.-butoxy-carbonyl)-amidocyclohexyl-Phosphoramidate-N-(L-alanine-L-alanine-tert.-butylester)-O-4-nitrophenyl.

### O-5-(tert.-butoxy-carbonyl)-amidocvclohexvl-Phosphoramidate-N-(L-alanine-L-alanine-tert.-butylester)-O-SN38

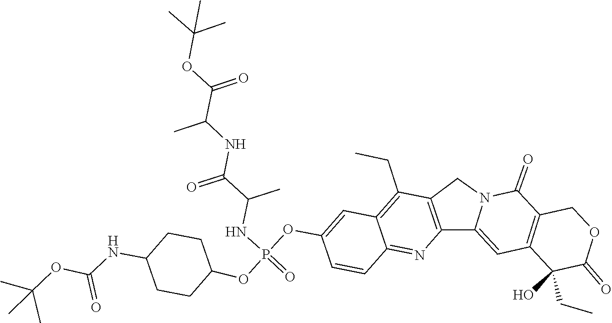 [SMILES: CCc1c2c(nc3ccc(OP(=O)(NC(C)C(=O)NC(C)C(=O)OC(C)(C)C)OC4CCC(NC(=O)OC(C)(C)C)CC4)cc13)-c1cc3c(c(=O)n1C2)COC(=O)[C@]3(O)CC]

The title compound was synthesized in accordance with general Method 6 from 6.4 mg SN38 (0.016 mmol). The product was obtained as yellow solid after preparative HPLC (Method C) and lyophilization. (12.2 mg, 0.014 mmol, 88%). HR-MS for C₄₃H₅₉N₅O₁₂P⁺ [M+H]⁺ calcd.: 868.3892, found 868.3931.

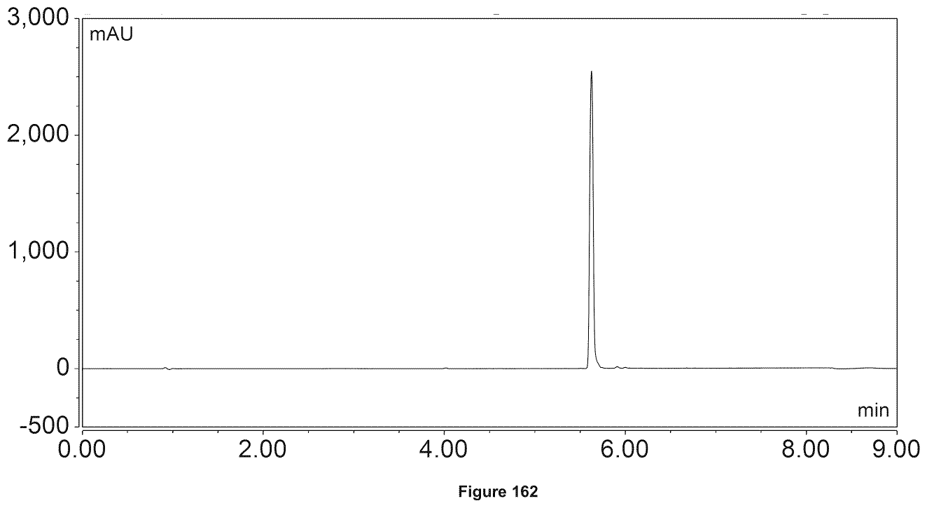

FIG. 162 shows an HPLC/UV chromatogram of the compound O-5-(tert.-butoxy-carbonyl)-amidocyclohexyl-Phosphoramidate-N-(L-alanine-L-alanine-tert.-butylester)-O—SN38.

### O—P5(PEG24)-amidocyclohexyl-Phosphoramidate-N-(L-alanine-L-alanine)-O-SN38

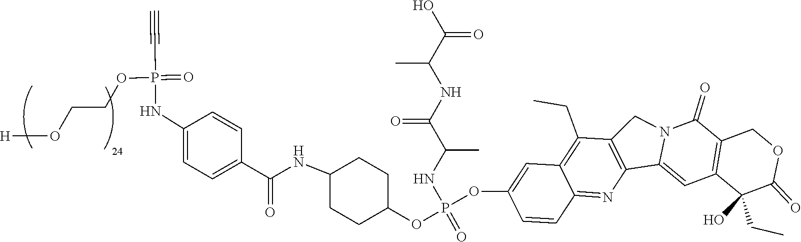

The title compound was synthesized in accordance to general Method 5 for Boc- and tert-butyl deprotection followed by amide coupling from 6.4 mg of O-5-(tert.-butoxy-carbonyl)-amidocyclohexyl-Phosphoramidate-N-(L-alanine-L-alanine-tert.-butylester)-O—SN38 (0.009 mmol, 1 eq). The product was obtained as yellowish oil after preparative HPLC (Method C) and lyophilization. (8.6 mg, 0.004 mmol, 48% over two steps). HR-MS for C₉₁H₁₄₆N₆O₃₇P₂²⁺ [M+2H]²⁺ calcd.: 988.4596, found 988.4565.

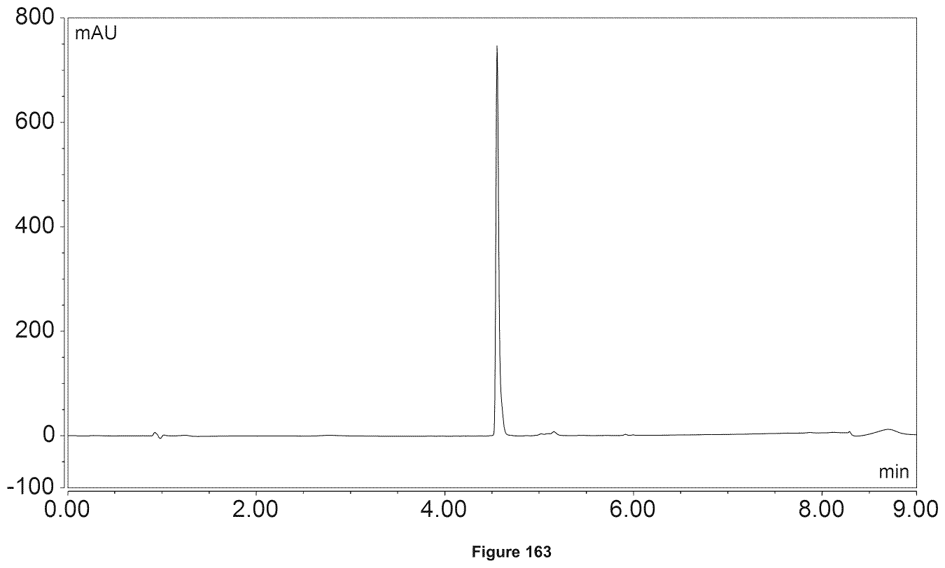

FIG. 163 shows an HPLC/UV chromatogram of the compound O—P5(PEG24)-amidocyclohexyl-Phosphoramidate-N-(L-alanine-L-alanine)-O—SN38.

### O-5-(tert.-butoxy-carbonyl)-amidocyclohexyl-Phosphoramidate-N-(L-alanine-tert.-butylester)-O-4-nitrophenyl

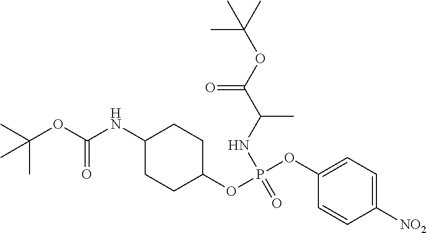 [SMILES: CC(NP(=O)(Oc1ccc([N+](=O)[O-])cc1)OC1CCC(NC(=O)OC(C)(C)C)CC1)C(=O)OC(C)(C)C]

The title compound was synthesized in accordance with the general Method 3 for the synthesis of nitrophenyl phosphoramidates from 269 mg 4-nitrophenyl phosphorodichloridate (1.05 mmol), 215 mg 4-(Boc-amino)cyclohexanol (1.0 mmol) and 200 mg L-alanine tert.-butyl ester hydrochloride (1.1 mmol). The product was obtained as white solid (421 mg, 0.67 mmol, 78%). MS for C₂₄H₃₉N₃O₉P⁺ [M+H]⁺ calcd.: 544.2 found, 544.4.

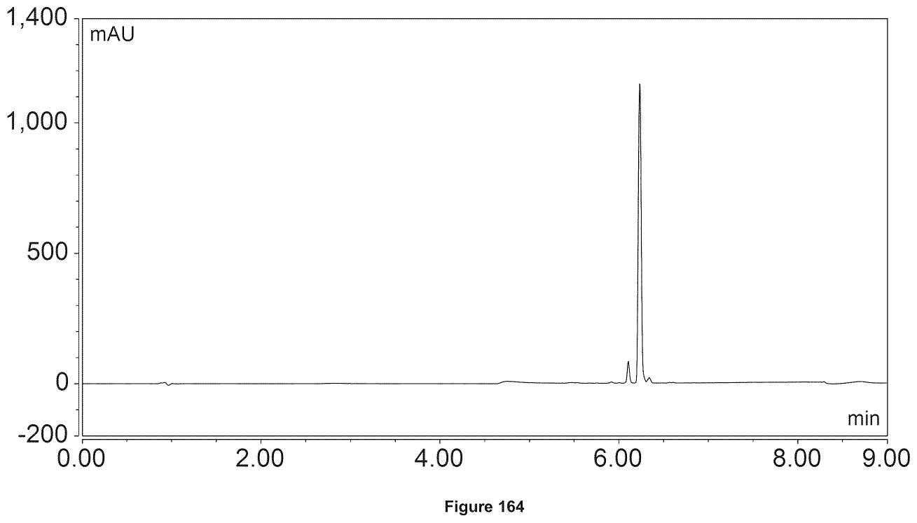

FIG. 164 shows an HPLC/UV chromatogram of the compound O-5-(tert.-butoxy-carbonyl)-amidocyclohexyl-Phosphoramidate-N-(L-alanine-tert.-butylester)-O-4-nitrophenyl.

### O-5-(tert.-butoxy-carbonyl)-amidocyclohexyl-Phosphoramidate-N-(L-alanine-tert.-butylester)-O-SN38

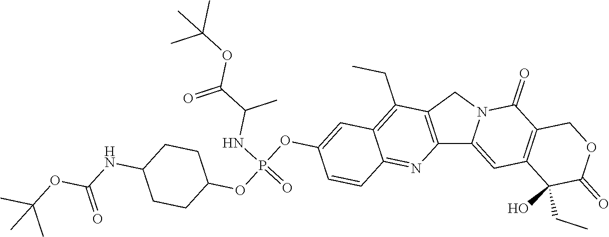 [SMILES: CCc1c2c(nc3ccc(OP(=O)(NC(C)C(=O)OC(C)(C)C)OC4CCC(NC(=O)OC(C)(C)C)CC4)cc13)-c1cc3c(c(=O)n1C2)COC(=O)[C@]3(O)CC]

The title compound was synthesized in accordance with general Method 6 from 6.1 mg SN38 (0.016 mmol). The product was obtained as yellow solid after preparative HPLC (Method C) and lyophilization. (10.6 mg, 0.013 mmol, 81%). HR-MS for C₄₀H₅₄N₄O₁₁P⁺ [M+H]⁺ calcd.: 797.3521, found 797.3552.

### O—P5(PEG24)-amidocvclohexvl-Phosphoramidate-N-(L-alanine-tert.-butylester)-O-SN38

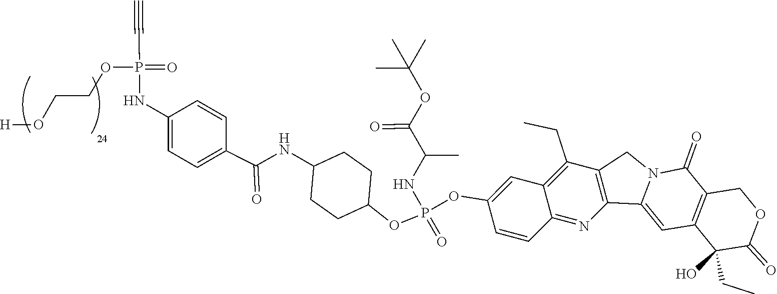

The title compound was synthesized in accordance to general Method 4 for Boc deprotection in the presence of the tert-butyl ester followed by amide coupling from 10.6 mg of O-5-(tert.-butoxy-carbonyl)-amidocyclohexyl-Phosphoramidate-N-(L-alanine-tert.-butylester)-O—SN38 (0.013 mmol, 1 eq). The product was obtained as yellowish oil after preparative HPLC (Method C) and lyophilization. (16.3 mg, 0.008 mmol, 62% over two steps). HR-MS for C₉₂H₁₄₉N₅O₃₆P₂²⁺ [M+2H]²⁺ calcd.: 980.9724, found 980.9706

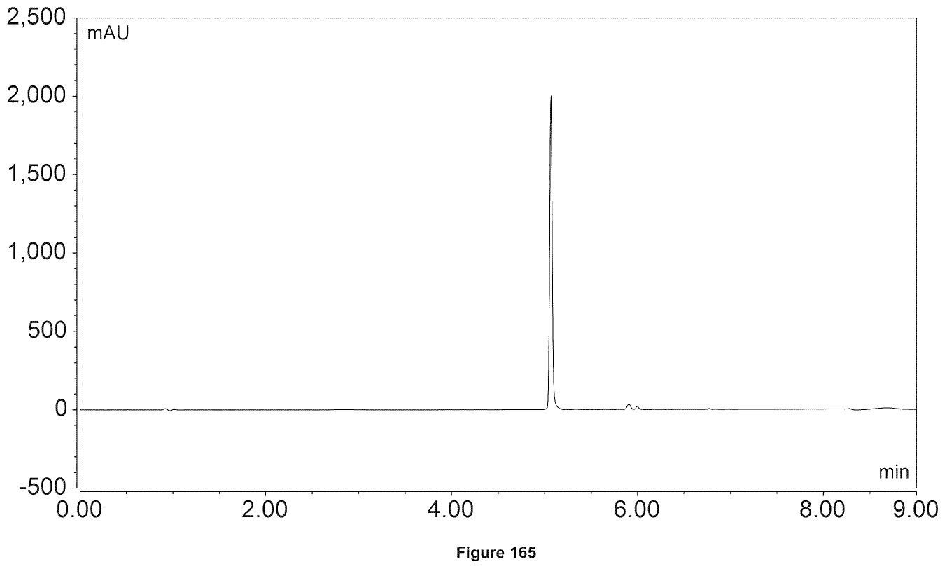

FIG. 165 shows an HPLC/UV chromatogram of the compound O—P5(PEG24)-amidocyclohexyl-Phosphoramidate-N-(L-alanine-tert.-butylester)-O—SN38.

### O-5-(tert.-butoxy-carbonyl)-amidocvclohexvl-Phosphoramidate-N-(L-alanine-iso-propvlester)-O-4-nitrophenyl

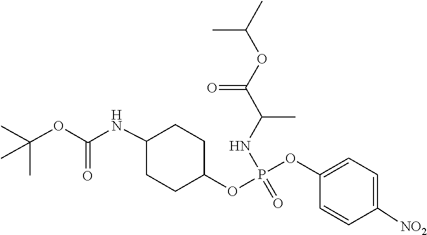

The title compound was synthesized in accordance with the general Method 3 for the synthesis of nitrophenyl phosphoramidates from 269 mg 4-nitrophenyl phosphorodichloridate (1.05 mmol), 215 mg 4-(Boc-amino)cyclohexanol (1.0 mmol) and 184 mg L-alanine isopropyl ester hydrochloride (1.1 mmol). The product was obtained as white solid (402 mg, 0.67 mmol, 76%). MS for C₂₃H₃₇N₃O₉P⁺ [M+H]⁺ calcd.: 530.2 found, 530.3.

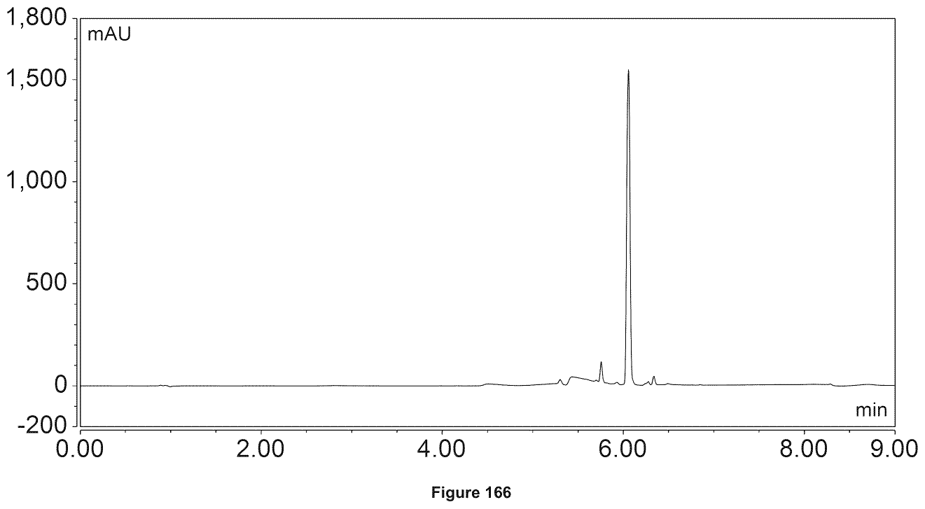

FIG. 166 shows an HPLC/UV chromatogram of the compound O-5-(tert.-butoxy-carbonyl)-amidocyclohexyl-Phosphoramidate-N-(L-alanine-iso-propylester)-O-4-nitrophenyl.

### O-5-(tert.-butoxy-carbonyl)-amidocvclohexvl-Phosphoramidate-N-(L-alanine-iso-propvlester)-O-SN38

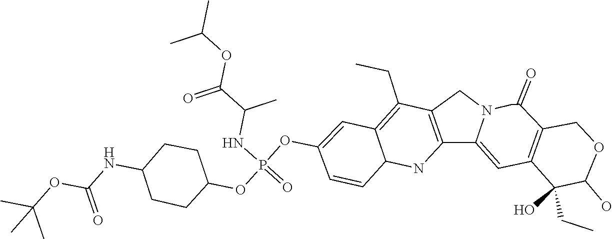

The title compound was synthesized in accordance with general Method 6 from 7.6 mg SN38 (0.019 mmol). The product was obtained as yellow solid after preparative HPLC (Method C) and lyophilization. (11.0 mg, 0.014 mmol, 74%). HR-MS for C₃₉H₅₂N₄O₁₁P⁺ [M+H]⁺ calcd.: 783.3365, found 783.3359.

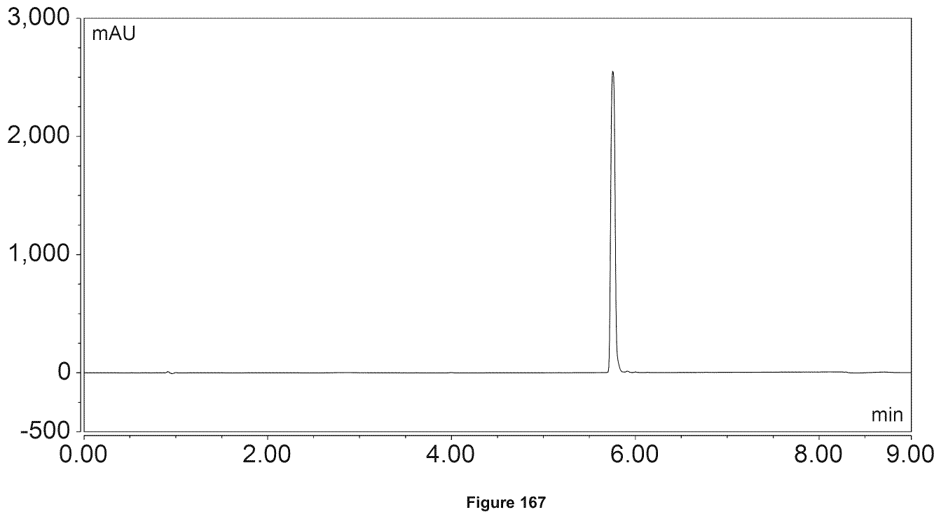

FIG. 167 shows an HPLC/UV chromatogram of the compound O-5-(tert.-butoxy-carbonyl)-amidocyclohexyl-Phosphoramidate-N-(L-alanine-iso-propylester)-O—SN38.

### O—P5(PEG24)-amidocyclohexyl-Phosphoramidate-N-(L-alanine-iso-propvlester)-O-SN38

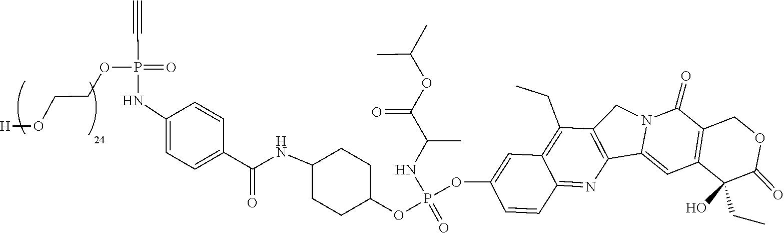

The title compound was synthesized in accordance to general Method 4 for Boc deprotection followed by amide coupling from 11 mg of O-5-(tert.-butoxy-carbonyl)-amidocyclohexyl-Phosphoramidate-N-(L-alanine-iso-propylester)-O—SN38 (0.014 mmol, 1 eq). The product was obtained as yellowish oil after preparative HPLC (Method C) and lyophilization. (22.7 mg, 0.012 mmol, 85% over two steps). HR-MS for C₉₁H₁₄₇N₅O₃₆P₂²⁺ [M+2H]²⁺ calcd.: 973.9645, found 973.9664.

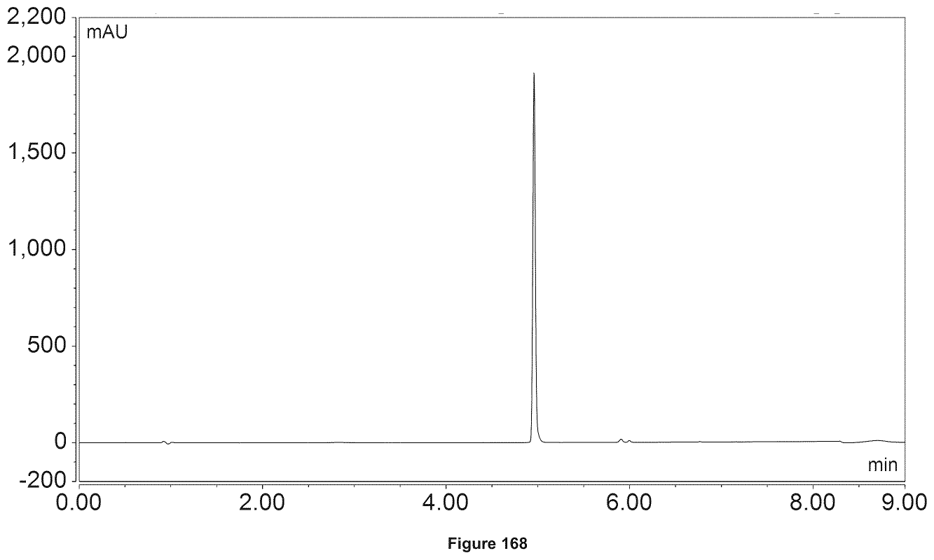

FIG. 168 shows an HPLC/UV chromatogram of the compound O—P5(PEG24)-amidocyclohexyl-Phosphoramidate-N-(L-alanine-iso-propylester)-O—SN38.

### O-5-(tert.-butoxy-carbonyl)-amidocyclohexyl-Phosphoramidate-N-(2,2-aminobutyric acid-tert.-butylester)-O-4-nitrophenyl

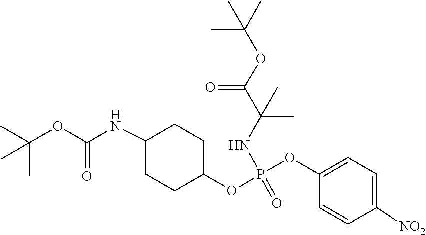 [SMILES: CC(C)(C)OC(=O)NC1CCC(OP(=O)(NC(C)(C)C(=O)OC(C)(C)C)Oc2ccc([N+](=O)[O-])cc2)CC1]

The title compound was synthesized in accordance with the general Method 3 for the synthesis of nitrophenyl phosphoramidates from 269 mg 4-nitrophenyl phosphorodichloridate (1.05 mmol), 215 mg 4-(Boc-amino)cyclohexanol (1.0 mmol) and 175 mg aminoisobutyric acid tert.-butyl ester (1.1 mmol). The product was obtained as white solid (346 mg, 0.67 mmol, 78%). MS for C₂₅H₄₁N₃O₉P⁺ [M+H]⁺ calcd.: 558.3 found, 558.4.

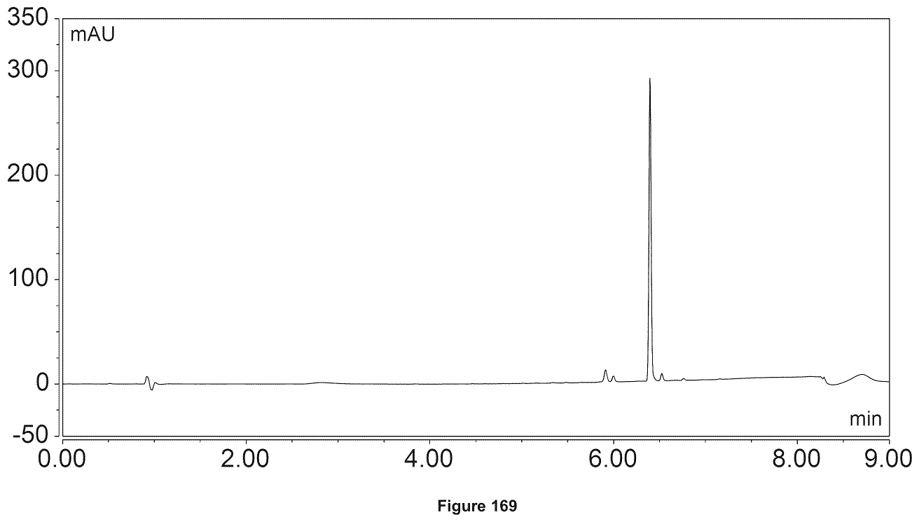

FIG. 169 shows an HPLC/UV chromatogram of the compound O-5-(tert.-butoxy-carbonyl)-amidocyclohexyl-Phosphoramidate-N-(2,2-aminobutyric acid-tert.-butylester)-O-4-nitrophenyl.

### O-5-(tert.-butoxy-carbonyl)-amidocyclohexyl-Phosphoramidate-N-(2,2-aminobutyric acid-tert.-butylester)-O-SN38

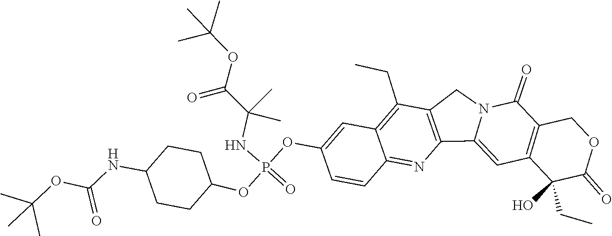 [SMILES: CCc1c2c(nc3ccc(OP(=O)(NC(C)(C)C(=O)OC(C)(C)C)OC4CCC(NC(=O)OC(C)(C)C)CC4)cc13)-c1cc3c(c(=O)n1C2)COC(=O)[C@]3(O)CC]

The title compound was synthesized in accordance with general Method 6 from 7.1 mg SN38 (0.018 mmol). The product was obtained as yellow solid after preparative HPLC (Method C) and lyophilization. (6.3 mg, 0.008 mmol, 44%). MS for C₄₁H₅₆N₄O₁₁P⁺ [M+H]⁺ calcd.: 811.4, found 811.4.

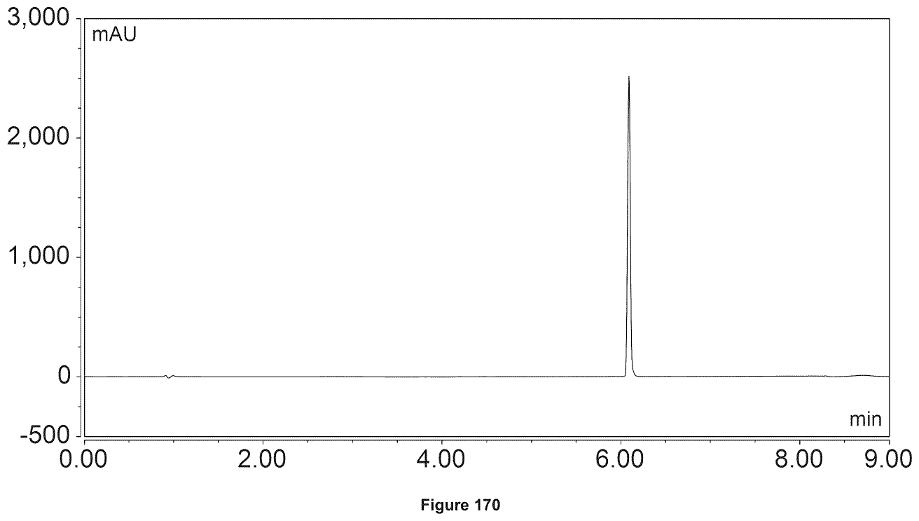

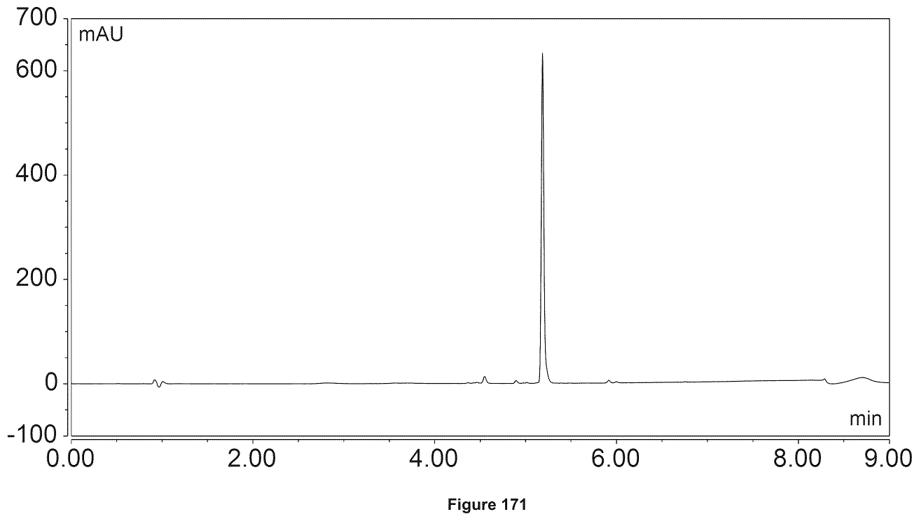

FIG. 170 shows an HPLC/UV chromatogram of the compound O-5-(tert.-butoxy-carbonyl)-amidocyclohexyl-Phosphoramidate-N-(2,2-aminobutyric acid-tert.-butylester)-O—SN38.

### O—P5(PEG24)-amidocyclohexyl-Phosphoramidate-N-(2,2-aminobutyric acid-tert.-butylester)-O—SN38

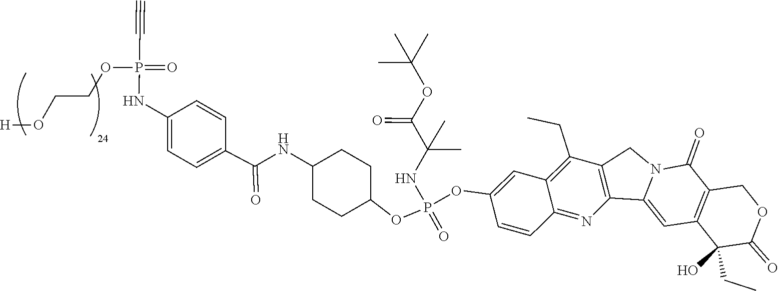

The title compound was synthesized in accordance to general Method 4 for Boc deprotection in the presence of the tert-butyl ester followed by amide coupling from 6.3 mg of O-(5-tert.-butoxy-carbonyl-aminocyclohexyl)-N-(L-alanine-tert.-butyl)-O—(SN38)-Phosphoramidate (0.008 mmol, 1 eq). The product was obtained as yellowish oil after preparative HPLC (Method C) and lyophilization. (10.7 mg, 0.005 mmol, 68% over two steps).

FIG. 171 shows an HPLC/UV chromatogram of the compound O—P5(PEG24)-amidocyclohexyl-Phosphoramidate-N-(2,2-aminobutyric acid-tert-butylester)-O—SN38.

### O-5-(tert.-butoxy-carbonyl)-amidopentyl-Phosphoramidate-N-(2-tert.-butyl-disulfide-ethyl)-O-4-nitrophenyl

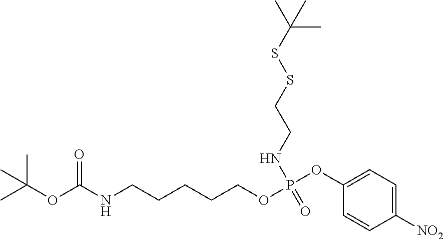 [SMILES: CC(C)(C)OC(=O)NCCCCCOP(=O)(NCCSSC(C)(C)C)Oc1ccc([N+](=O)[O-])cc1]

The title compound was synthesized in accordance with the general Method 3 for the synthesis of nitrophenyl phosphoramidates from 250 mg 4-nitrophenyl phosphorodichloridate (0.98 mmol), 199 mg Boc-aminopentanol (0.98 mmol) and 165 mg cysteamine tert.-butyl disulfide (0.98 mmol). The product was obtained as white solid (215 mg, 0.4 mmol, 40%). MS for C₂₂H₃₉N₃O₇PS₂⁺ [M+H]⁺ calcd.: 552.2, found 552.3.

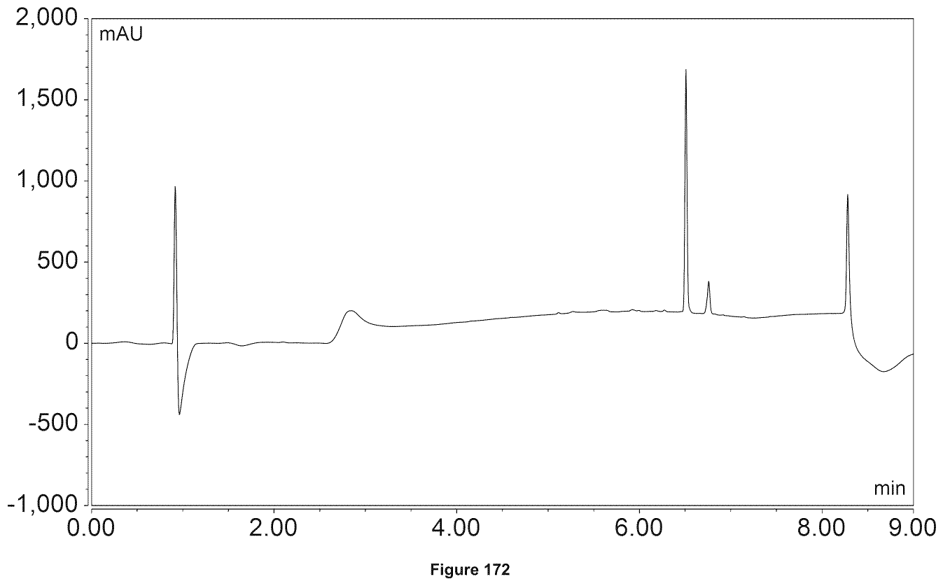

FIG. 172 shows an HPLC/UV chromatogram of the compound O-5-(tert.-butoxy-carbonyl)-amidopentyl-Phosphoramidate-N-(2-tert.-butyl-disulfide-ethyl)-O-4-nitrophenyl.

### O-5-(tert.-butoxy-carbonyl)-amidopentyl-Phosphoramidate-N-(2-tert.-butyl-disulfide-ethyl)-SN38

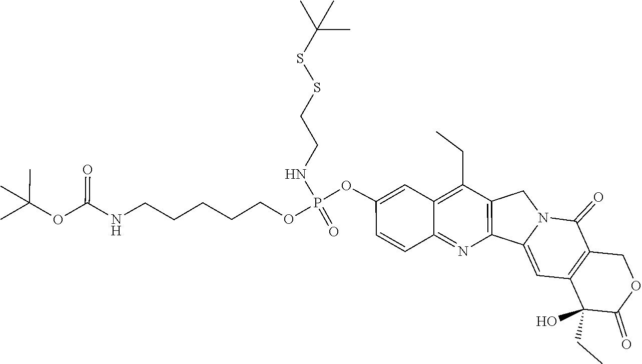 [SMILES: CCc1c2c(nc3ccc(OP(=O)(NCCSSC(C)(C)C)OCCCCCNC(=O)OC(C)(C)C)cc13)-c1cc3c(c(=O)n1C2)COC(=O)[C@]3(O)CC]

The title compound was synthesized in accordance with general Method 6 from 5 mg SN38 (0.013 mmol) and 21 mg O-5-(tert.-butoxy-carbonyl)-amidopentyl-Phosphoramidate-N-(2-tert.-butyl-disulfide-ethyl)-O-4-nitrophenyl (0.04 mmol). The product was obtained as yellow solid after preparative HPLC (Method C) and lyophilization. (9.0 mg, 0.011 mmol, 84%). HR-MS for C₃₈H₅₄N₄O₉PS₂⁺ [M+H]⁺ calcd.: 805.3064 found 805.3097.

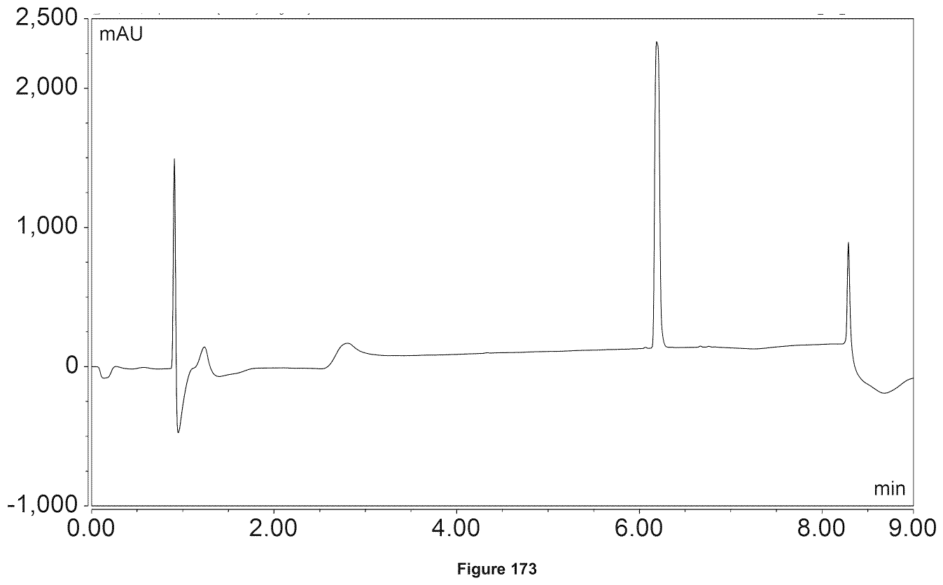

FIG. 173 shows an HPLC/UV chromatogram of the compound O-5-(tert.-butoxy-carbonyl)-amidopentyl-Phosphoramidate-N-(2-tert.-butyl-disulfide-ethyl)-SN38.

### O—P5(PEG24)-amidopentyl-Phosphoramidate-N-(2-tert.-butyl-disulfide-ethyl)-SN38

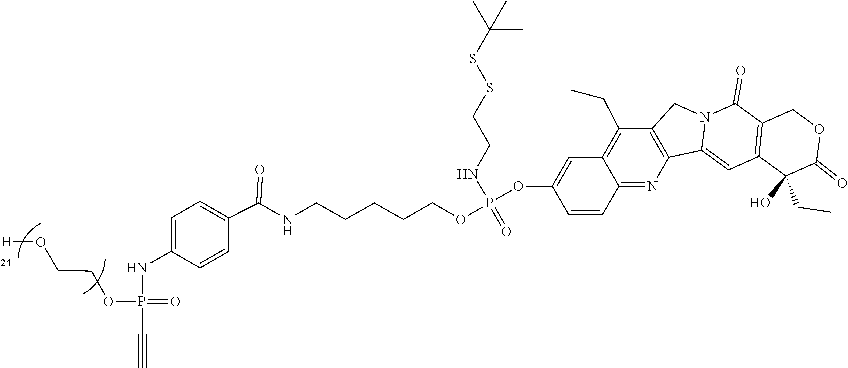

The title compound was synthesized in accordance to general Method 4 for Boc deprotection in the presence of the tert-butyl ester followed by amide coupling from 8.1 mg of O-5-(tert.-butoxy-carbonyl)-amidopentyl-Phosphoramidate-N-(2-tert.-butyl-disulfide-ethyl)-SN38 (0.010 mmol, 1 eq). The product was obtained as yellowish oil after preparative HPLC (Method C) and lyophilization. (13.0 mg, 0.007 mmol, 70% over two steps). HR-MS for C₉₀H₁₄₉N₅O₃₄P₂S₂²⁺ [M+2H]²⁺ calcd.: 984.9495, found 984.9486.

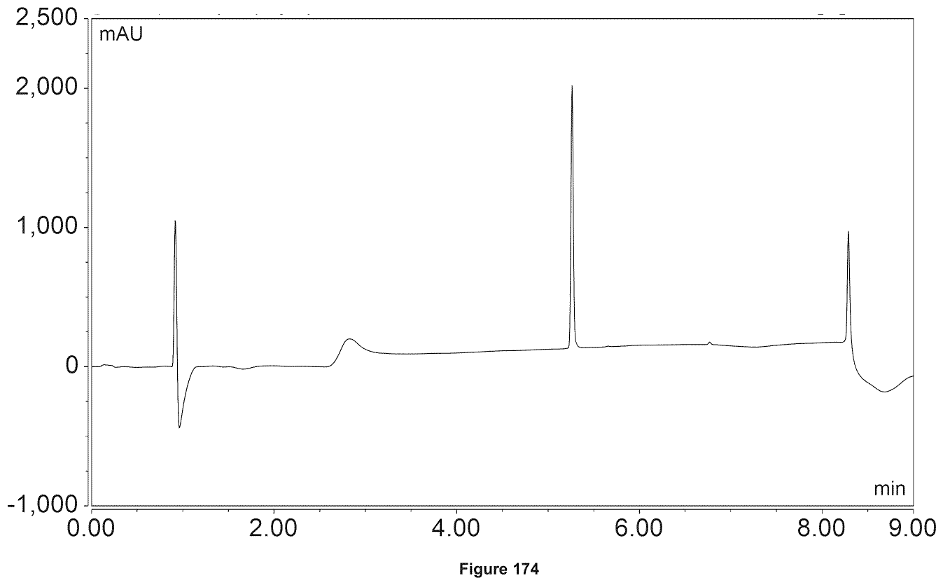

FIG. 174 shows an HPLC/UV chromatogram of the compound O—P5(PEG24)-amidopentyl-Phosphoramidate-N-(2-tert.-butyl-disulfide-ethyl)-SN38.

### O-5-(tert.-butoxy-carbonyl)-amidopentyl-Phosphoramidate-N-(2-tert.-butyl-disulfide-ethyl)-DXD

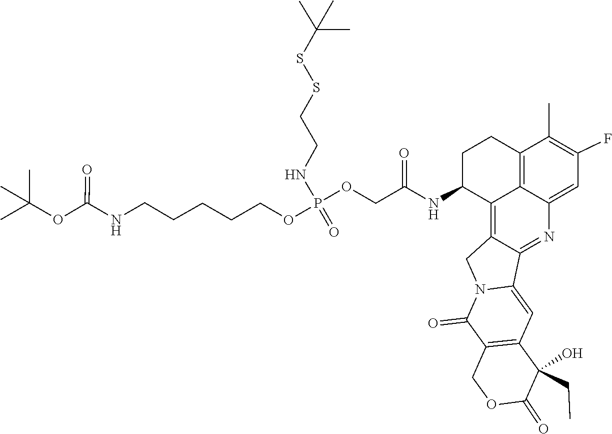 [SMILES: CC[C@@]1(O)C(=O)OCc2c1cc1n(c2=O)Cc2c-1nc1cc(F)c(C)c3c1c2[C@@H](NC(=O)COP(=O)(NCCSSC(C)(C)C)OCCCCCNC(=O)OC(C)(C)C)CC3]

The title compound was synthesized in accordance with general Method 7 from 5 mg Dxd (0.01 mmol) and 16.8 mg O-5-(tert.-butoxy-carbonyl)-amidopentyl-Phosphoramidate-N-(2-tert.-butyl-disulfide-ethyl)-O-4-nitrophenyl (0.03 mmol). The product was obtained as yellow solid after preparative HPLC (Method C) and lyophilization. (8.1 mg, 0.009 mmol, 89%). HR-MS for C₄₂H₅₈FN₅O₁₀PS₂⁺ [M+H]⁺ calcd.: 906.3341 found 906.3363.

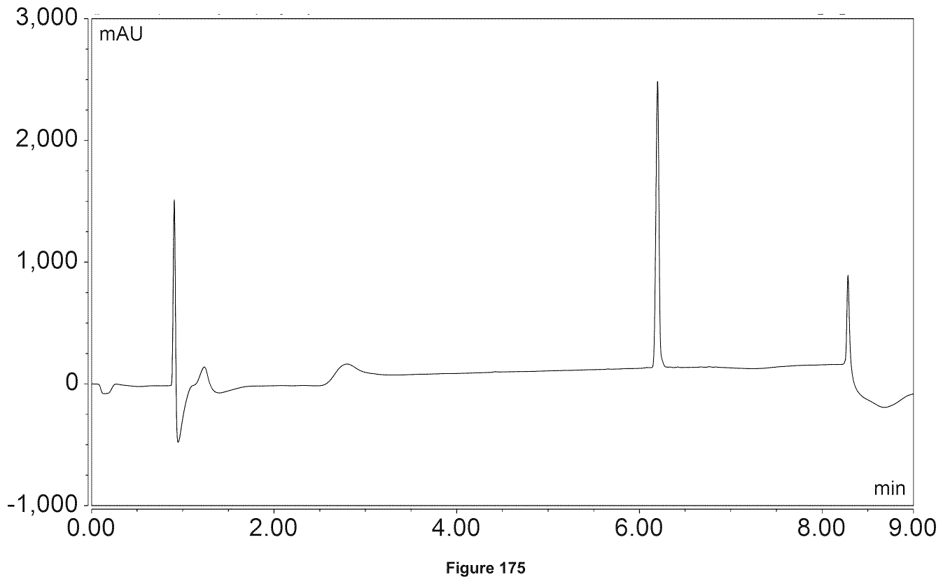

FIG. 175 shows an HPLC/UV chromatogram of the compound O-5-(tert.-butoxy-carbonyl)-amidopentyl-Phosphoramidate-N-(2-tert.-butyl-disulfide-ethyl)-DXD.

### O—P5(PEG24)-amidopentyl-Phosphoramidate-N-(2-tert.-butyl-disulfide-ethyl)-DXD

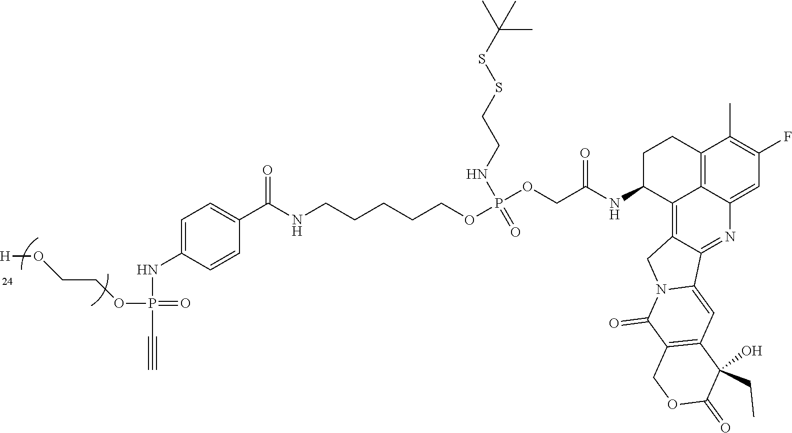

The title compound was synthesized in accordance to general Method 4 for Boc deprotection in the presence of the tert-butyl ester followed by amide coupling from 8.1 mg of O-5-(tert.-butoxy-carbonyl)-amidopentyl-Phosphoramidate-N-(2-tert.-butyl-disulfide-ethyl)-DXD (0.009 mmol, 1 eq). The product was obtained as yellowish oil after preparative HPLC (Method C) and lyophilization. (7.9 mg, 0.004 mmol, 44% over two steps). HR-MS for C₉₄H₁₅₃N₆O₃₅P₂S₂²⁺ [M+2H]²⁺ calcd.: 1035.9650, found 1035.9632.

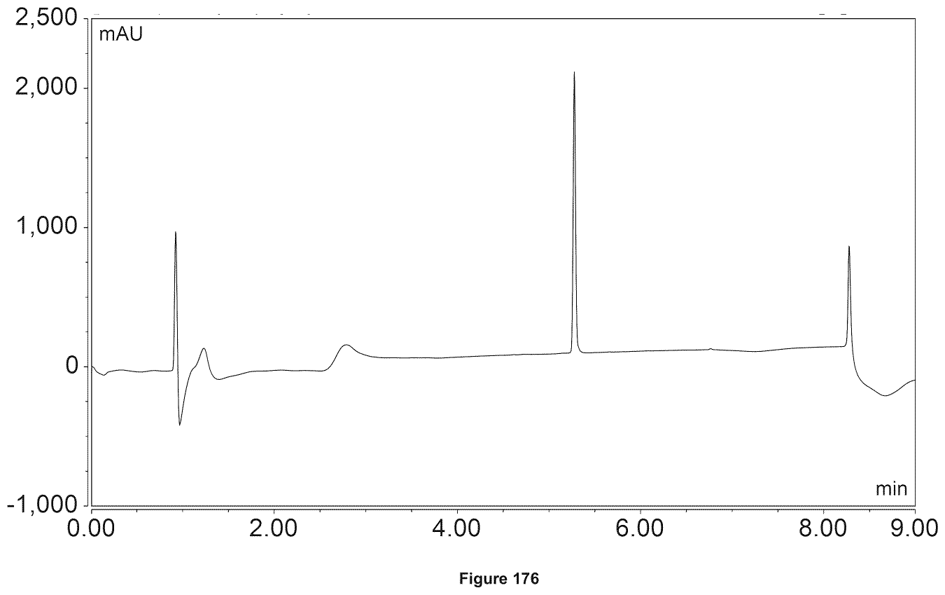

FIG. 176 shows an HPLC/UV chromatogram of the compound O—P5(PEG24)-amidopentyl-Phosphoramidate-N-(2-tert.-butyl-disulfide-ethyl)-DXD.

### O-5-(phenylmethoxy-carbonyl)-amidopentyl-Phosphoramidate-N-(2-2-Diethoxy-ethyl)-O-4-nitrophenyl

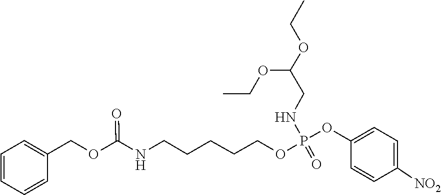

The title compound was synthesized in accordance with the general Method 3 for the synthesis of nitrophenyl phosphoramidates from 256 mg 4-nitrophenyl phosphorodichloridate (1.0 mmol), 237 mg Cbz-aminopentanol (1.0 mmol) and 133 mg aminoacetaldehyde diethyl acetal (1.0 mmol). The product was obtained as white solid (514 mg, 0.98 mmol, 98%). MS for C₂₂H₃₉N₃O₉P⁺ [M+H]⁺ calcd.: 554.2, found 554.2.

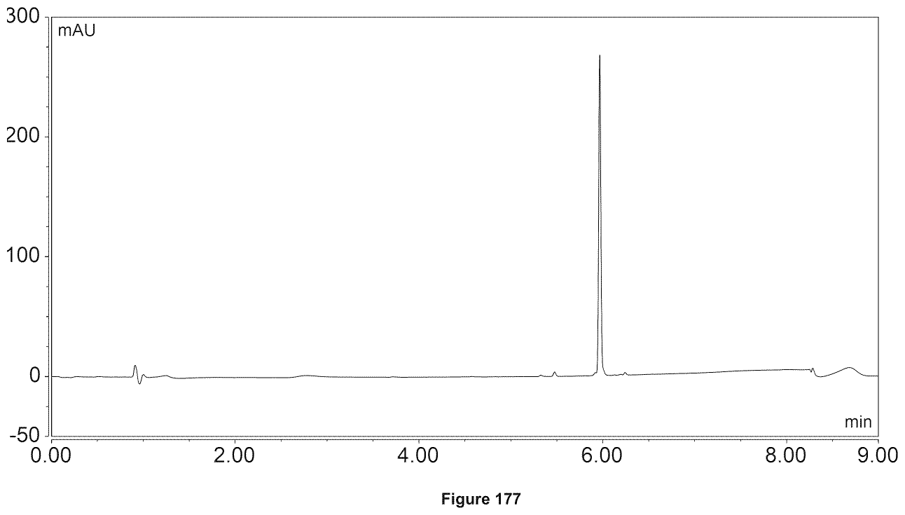

FIG. 177 shows an HPLC/UV chromatogram of the compound O-5-(phenylmethoxy-carbonyl)-amidopentyl-Phosphoramidate-N-(2-2-Diethoxy-ethyl)-O-4-nitrophenyl.

### O-5-(phenylmethoxy-carbonyl)-amidopentyl-Phosphoramidate-N-(2-2-Diethoxy-ethyl)-O-SN38

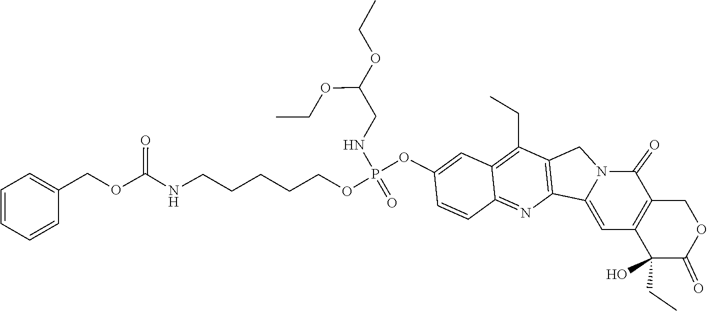 [SMILES: CCOC(CNP(=O)(OCCCCCNC(=O)OCc1ccccc1)Oc1ccc2nc3c(c(CC)c2c1)Cn1c-3cc2c(c1=O)COC(=O)[C@]2(O)CC)OCC]

The title compound was synthesized in accordance with general Method 6 from 4.6 mg SN38 (0.012 mmol) and 32.5 mg O-5-(phenylmethoxy-carbonyl)-amidopentyl-Phosphoramidate-N-(2-2-Diethoxy-ethyl)-O-4-nitrophenyl (0.059 mmol). The product was obtained as yellow solid after preparative HPLC (Method C) and lyophilization. (3.5 mg, 0.004 mmol, 36%).

### O—P5(PEG24)-amidopentyl-Phosphoramidate-N-(2-2-Diethoxy-ethyl)-O-SN38

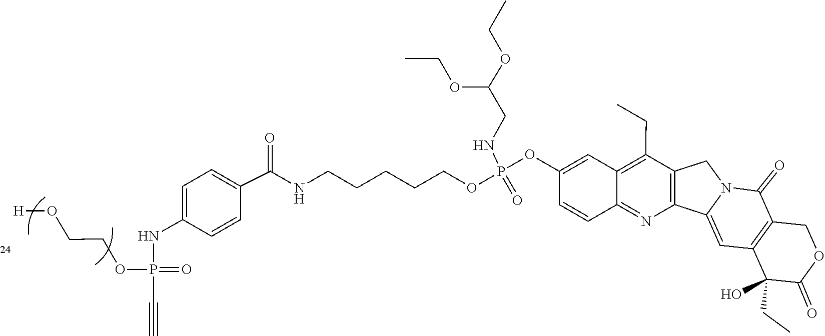

The title compound was synthesized from 7.3 mg of O-5-(phenylmethoxy-carbonyl)-amidopentyl-Phosphoramidate-N-(2-2-Diethoxy-ethyl)-O—SN38 (0.008 mmol, 1 eq), which was dissolved in 1 mL MeOH and 1 mg Pd/C and stirred under H2 atmosphere to remove the Cbz protecting group. After complete deprotection the reaction was filtered, and the solvent was removed under reduced pressure. The residue was dissolved in 0.5 ml DMSO. 1.5 eq. PyBop, dissolved in DMSO and 5.0 eq. DIPEA were added. After 5 minutes, 1.0 eq of P5-PEG24-COOH dissolved in DMSO was added and the mixture was stirred at RT for 1 h. The mixture was diluted with H2O/MeCN (2:1) and directly subjected to preparative HPLC purification. The product was obtained as yellowish oil after preparative HPLC (Method C) and lyophilization. (8.2 mg, 0.004 mmol, 50% over two steps).

### O-5-(phenylmethoxy-carbonyl)-amidopentyl-Phosphoramidate-N-(2-2-Diethoxy-ethyl)-O-DXD

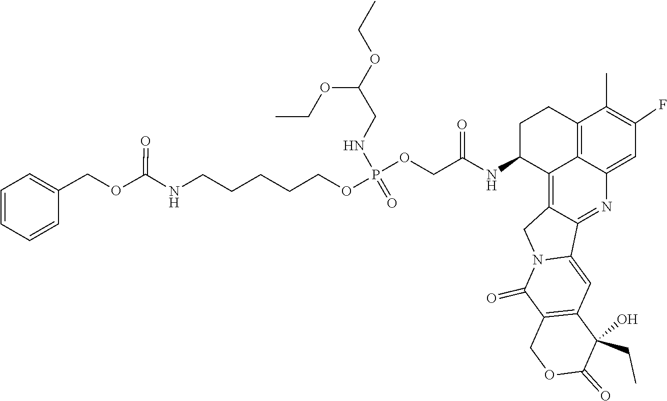 [SMILES: CCOC(CNP(=O)(OCCCCCNC(=O)OCc1ccccc1)OCC(=O)N[C@H]1CCc2c(C)c(F)cc3nc4c(c1c23)Cn1c-4cc2c(c1=O)COC(=O)[C@]2(O)CC)OCC]

The title compound was synthesized in accordance with general Method 7 from 5.8 mg Dxd (0.012 mmol) and 32.5 mg O-5-(phenylmethoxy-carbonyl)-amidopentyl-Phosphoramidate-N-(2-2-Diethoxy-ethyl)-O-4-nitrophenyl (0.059 mmol). The product was obtained as yellow solid after preparative HPLC (Method C) and lyophilization. (7.3 mg, 0.008 mmol, 68%).

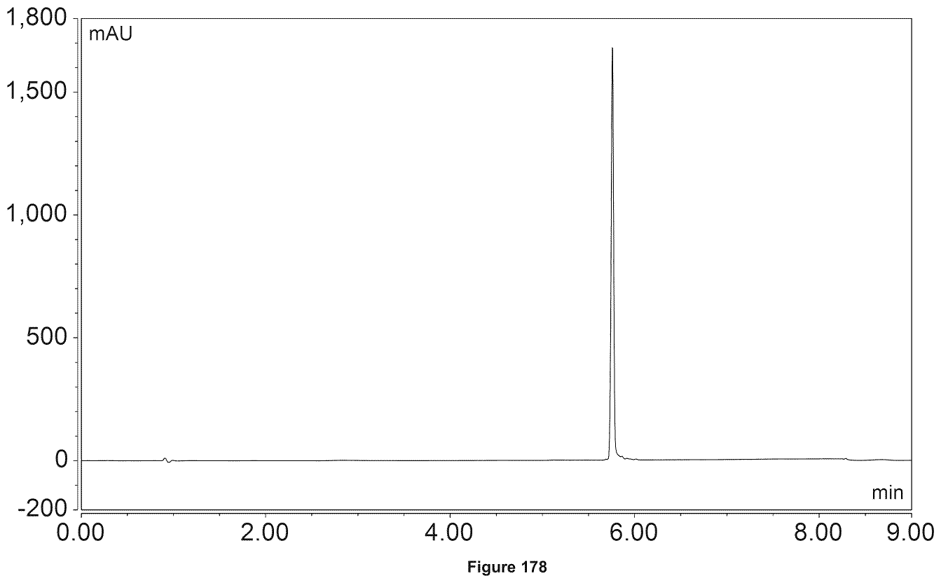

FIG. 178 shows an HPLC/UV chromatogram of the compound O-5-(phenylmethoxy-carbonyl)-amidopentyl-Phosphoramidate-N-(2-2-Diethoxy-ethyl)-O-DXD.

### O—P5(PEG24)-amidopentyl-Phosphoramidate-N-(2-2-Diethoxy-ethyl)-O-DXD

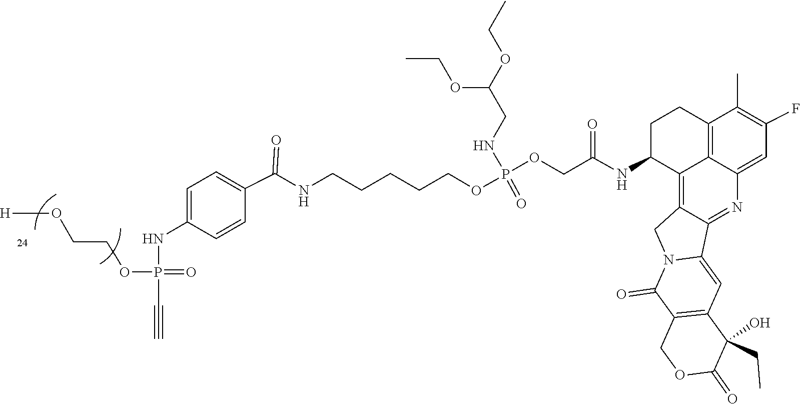

The title compound was synthesized from 7.3 mg of O-(5-tert.-butoxy-carbonyl-aminopentyl)-N-(aminoacetaldehyde diethyl acetal)-O-(DXd)-Phosphoramidate (0.008 mmol, 1 eq), which was dissolved in 1 mL MeOH and 1 mg Pd/C and stirred under H2 atmosphere to remove the Cbz protecting group. After complete deprotection the reaction was filtered, and the solvent was removed under reduced pressure. The residue was dissolved in 0.5 ml DMSO. 1.5 eq. PyBop, dissolved in DMSO and 5.0 eq. DIPEA were added. After 5 minutes, 1.0 eq of P5-PEG24-COOH dissolved in DMSO was added and the mixture was stirred at RT for 1 h. The mixture was diluted with H2O/MeCN (2:1) and directly subjected to preparative HPLC purification. The product was obtained as yellowish oil after preparative HPLC (Method C) and lyophilization. (8.2 mg, 0.004 mmol, 50% over two steps).

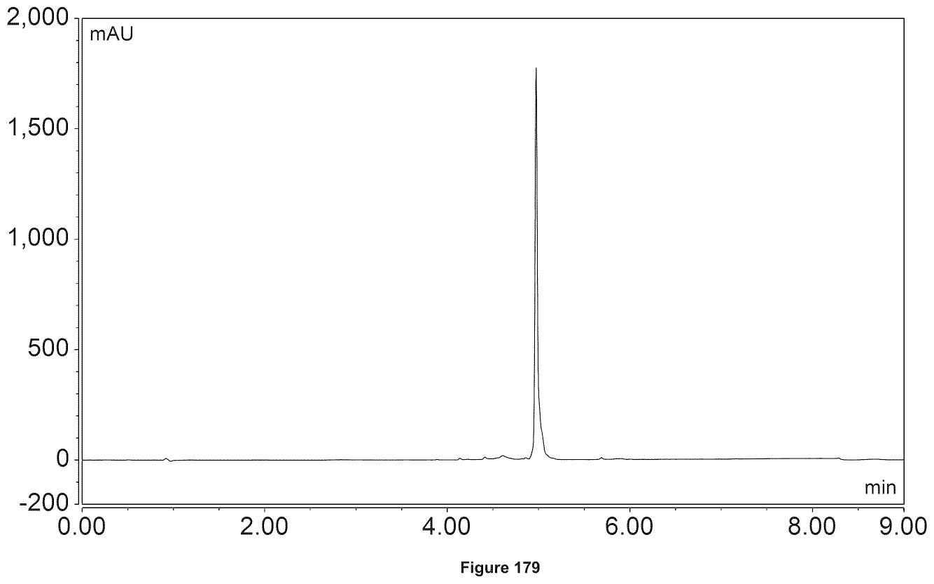

FIG. 179 shows an HPLC/UV chromatogram of the compound O—P5(PEG24)-amidopentyl-Phosphoramidate-N-(2-2-Diethoxy-ethyl)-O-DXD.

### O-5-(tert.-butoxy-carbonyl)-amidopentyl-Phosphoramidate-N-(L-alanine-L-alanine-tert.-butylester)-O-DHODH-IN-16

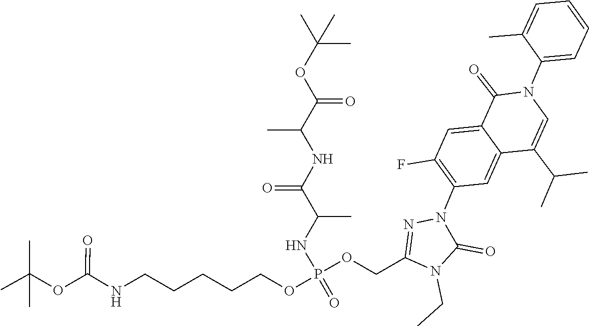 [SMILES: CCn1c(COP(=O)(NC(C)C(=O)NC(C)C(=O)OC(C)(C)C)OCCCCCNC(=O)OC(C)(C)C)nn(-c2cc3c(C(C)C)cn(-c4ccccc4C)c(=O)c3cc2F)c1=O]

The title compound was synthesized in accordance with general Method 6 from 5 mg DHODH-IN-16 (0.01 mmol) and 20 mg O-(5-tert.-butoxy-carbonyl-amidopentyl)-Phosphoramidate-N-(L-alanine-L-alanine-tert.-butylester)-O-4-nitrophenyl (0.033 mmol). The product was obtained as white solid after preparative HPLC (Method C) and lyophilization. (5.3 mg, 0.0058 mmol, 51%). MS for C₄₄H₆₄FN₇O₁₀P⁺ [M+H]⁺ calcd.: 900.4 found 900.5.

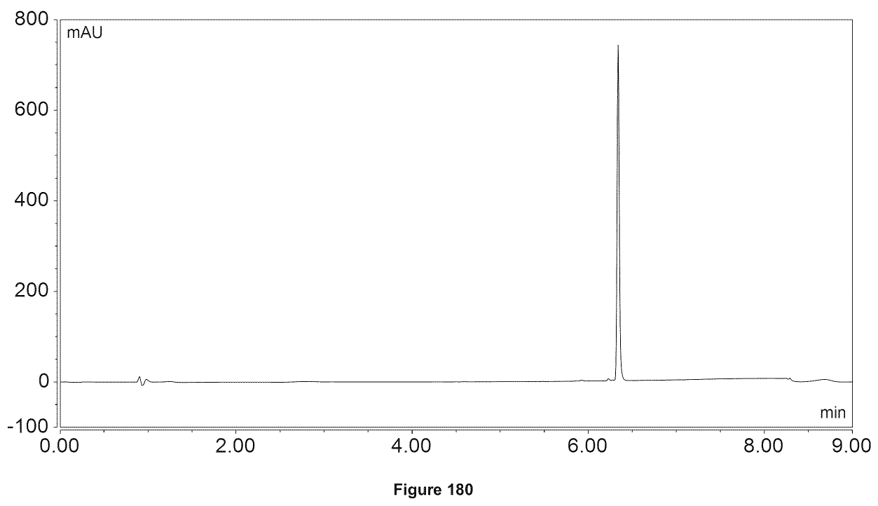

FIG. 180 shows an HPLC/UV chromatogram of the compound O-5-(tert.-butoxy-carbonyl)-amidopentyl-Phosphoramidate-N-(L-alanine-L-alanine-tert.-butylester)-O-DHODH-IN-16.

### O—P5(PEG24)-amidopentyl-Phosphoramidate-N-(L-alanine-L-alanine-tert.-butylester)-O-DHODH-IN-16

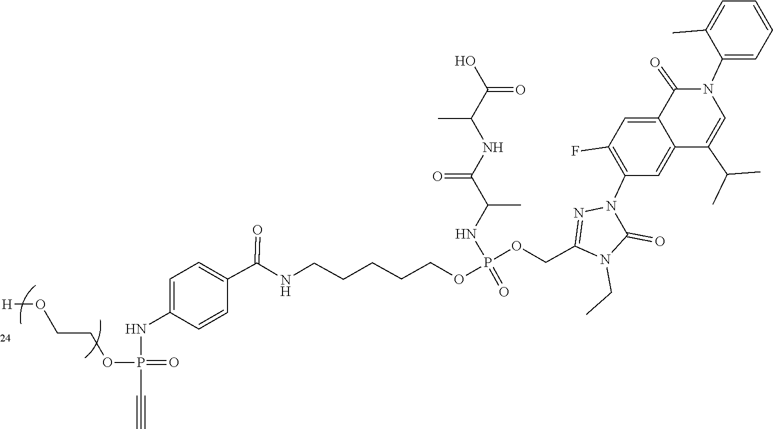

The title compound was synthesized in accordance to general Method 5 for Boc- and tert-butyl deprotection followed by amide coupling from 5.3 mg of O-(5-tert.-butoxy-carbonyl-aminopentyl)-N-(L-alanine-L-alanine-tert.-butylester)-O-(DHODH-IN-16)-Phosphoramidate (0.0058 mmol, 1 eq). The product was obtained as yellowish oil after preparative HPLC (Method C) and lyophilization. (6.4 mg, 0.003 mmol, 52% over two steps). MS for C₉₂H₁₅₂FN₈O₃₅P₂³⁺ [M+3H]³⁺ calcd.: 669.9, found 670.0.

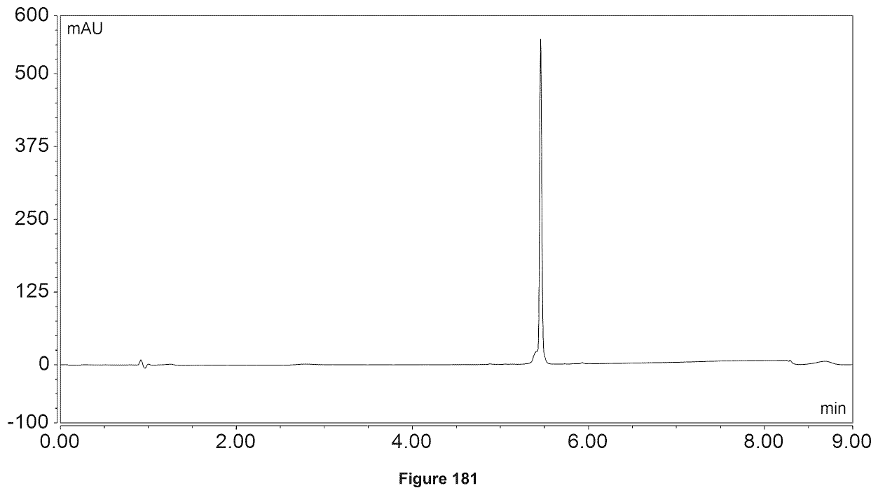

FIG. 181 shows an HPLC/UV chromatogram of the compound O—P5(PEG24)-amidopentyl-Phosphoramidate-N-(L-alanine-L-alanine-tert.-butylester)-O-DHODH-IN-16.

### O-5-(tert.-butoxy-carbonyl)-amidopentyl-Phosphoramidate-N-(L-alanine-L-alanine-tert.-butylester)-O-Roniciclib

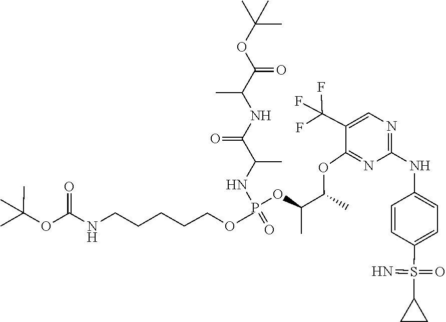

The title compound was synthesized in accordance with general Method 6 from 5 mg Roniciclib (0.012 mmol). The product was obtained as white solid after preparative HPLC (Method C) and lyophilization. (2 mg, 0.0022 mmol, 19%). MS for C₃₈H₆₁F₃N₇O₁₀PS²⁺ [M+2H]²⁺ calcd.: 447.7, found 447.7.

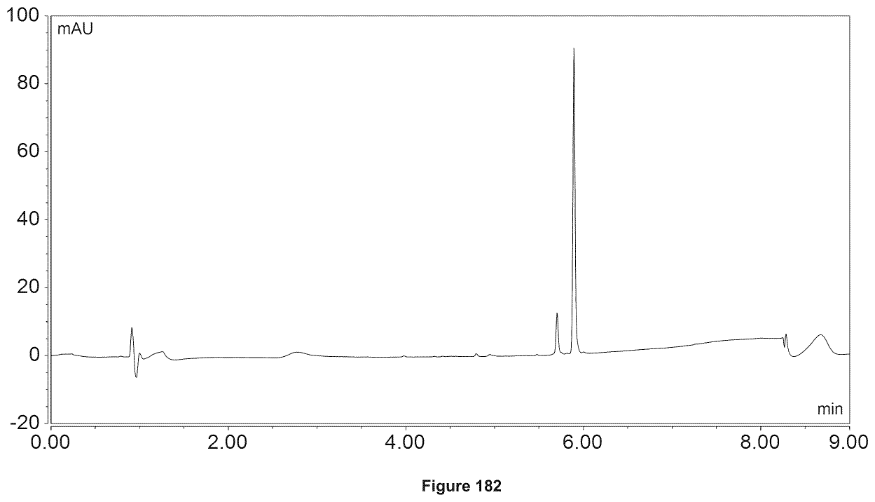

FIG. 182 shows an HPLC/UV chromatogram of the compound O-5-(tert.-butoxy-carbonyl)-amidopentyl-Phosphoramidate-N-(L-alanine-L-alanine-tert.-butylester)-O-Roniciclib.

### O—P5(PEG24)-amidopentyl-Phosphoramidate-N-(L-alanine-L-alanine-tert.-butylester)-O-Roniciclib

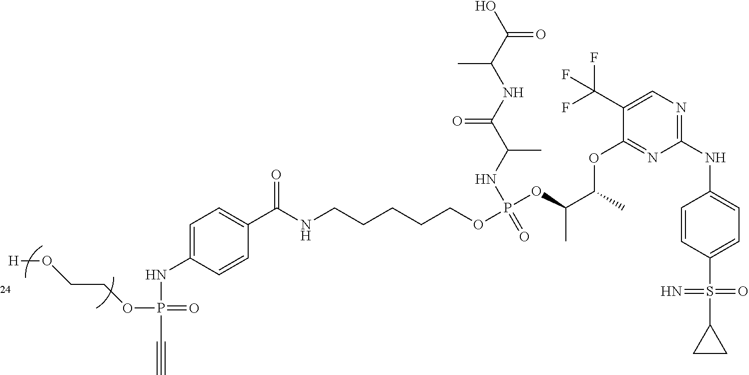

The title compound was synthesized in accordance to general Method 5 for Boc and tert-butyl ester deprotection followed by amide coupling from 2 mg of O-5-(tert.-butoxy-carbonyl)-amidopentyl-Phosphoramidate-N-(L-alanine-L-alanine-tert.-butylester)-O-Roniciclib (0.0022 mmol, 1 eq). The product was obtained as colorless oil after preparative HPLC (Method C) and lyophilization. (0.4 mg, 0.2 μmol, 10% over two steps). MS for C₈₆H₁₄₇F₃N₈O₃₅P₂²⁺ [M+2H]²⁺ calcd.: 1001.4, found 1001.6.

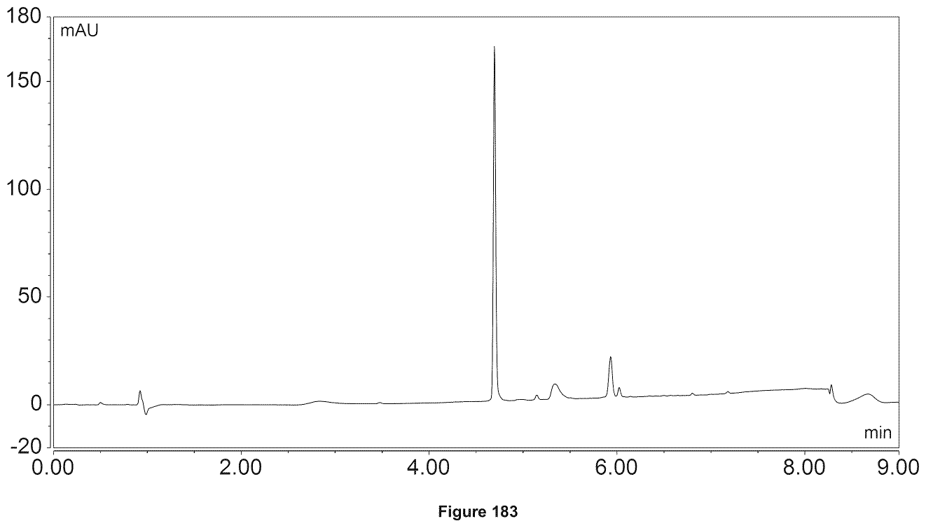

FIG. 183 shows an HPLC/UV chromatogram of the compound O—P5(PEG24)-amidopentyl-Phosphoramidate-N-(L-alanine-L-alanine-tert.-butylester)-O-Roniciclib.

### O-5-(tert.-butoxy-carbonyl)-amidopentyl-Phosphoramidate-N-(L-alanine-L-alanine-tert.-butylester)-O-Nampt-IN-1

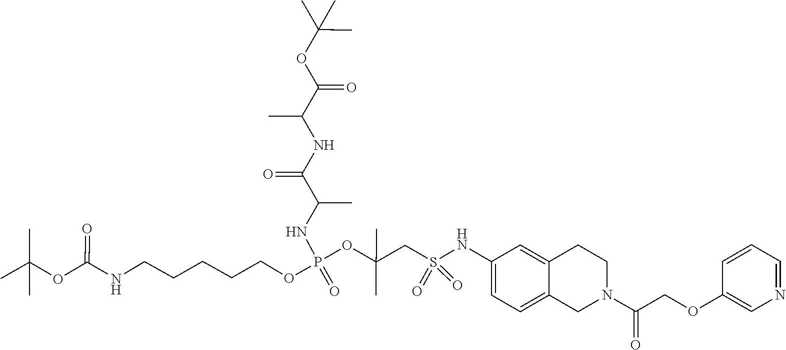 [SMILES: CC(NP(=O)(OCCCCCNC(=O)OC(C)(C)C)OC(C)(C)CS(=O)(=O)Nc1ccc2c(c1)CCN(C(=O)COc1cccnc1)C2)C(=O)NC(C)C(=O)OC(C)(C)C]

The title compound was synthesized in accordance with general Method 6 from 9 mg Nampt-IN-1 (0.021 mmol). The product was obtained as white solid after preparative HPLC (Method C) and lyophilization. (6.7 mg, 0.0076 mmol, 36%). MS for C₄₀H₆₄N₆O₁₂PS⁺ [M+H]⁺ calcd.: 883.4, found 883.4.

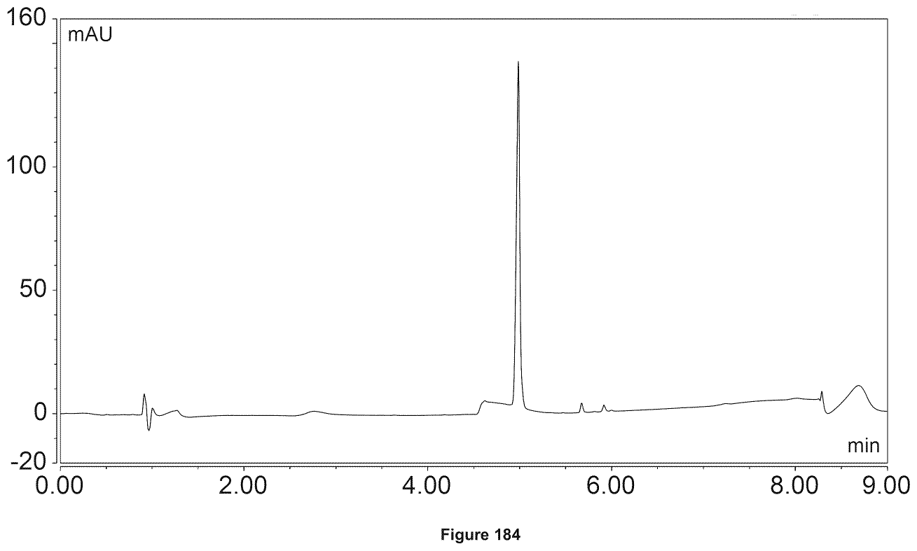

FIG. 184 shows an HPLC/UV chromatogram of the compound O-5-(tert.-butoxy-carbonyl)-amidopentyl-Phosphoramidate-N-(L-alanine-L-alanine-tert.-butylester)-O-Nampt-IN-1.

### O—P5(PEG24)-amidopentyl-Phosphoramidate-N-(L-alanine-L-alanine-tert.-butylester)-O-Nampt-IN-1

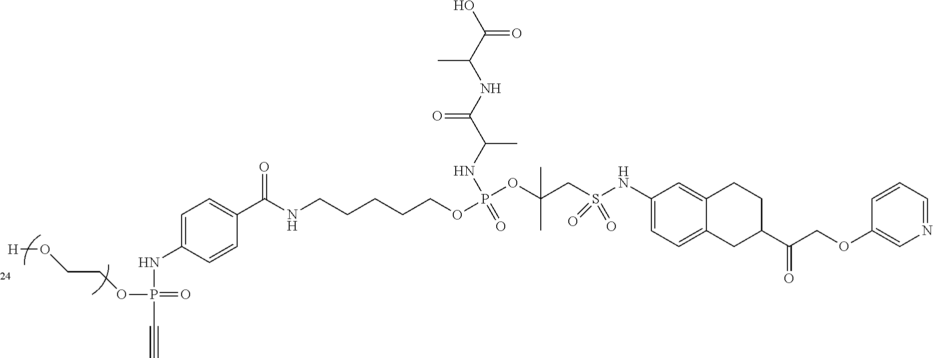

The title compound was synthesized in accordance with general Method 5 for Boc- and tert-butyl ester deprotection followed by amide coupling from 6.7 mg of O-(5-tert.-butoxy-carbonyl-aminopentyl)-N-(L-alanine-L-alanine-tert.-butylester)-O-(Nampt-IN-1)-Phosphoramidate (0.0076 mmol, 1 eq). The product was obtained as yellow oil after preparative HPLC (Method C) and lyophilization. (6.7 mg, 0.0034 mmol, 45% over two steps). MS for C₈₈H₁₅₁N₇O₃₇P₂S²⁺[M+2H]²⁺ calcd.: 996.0, found 996.1.

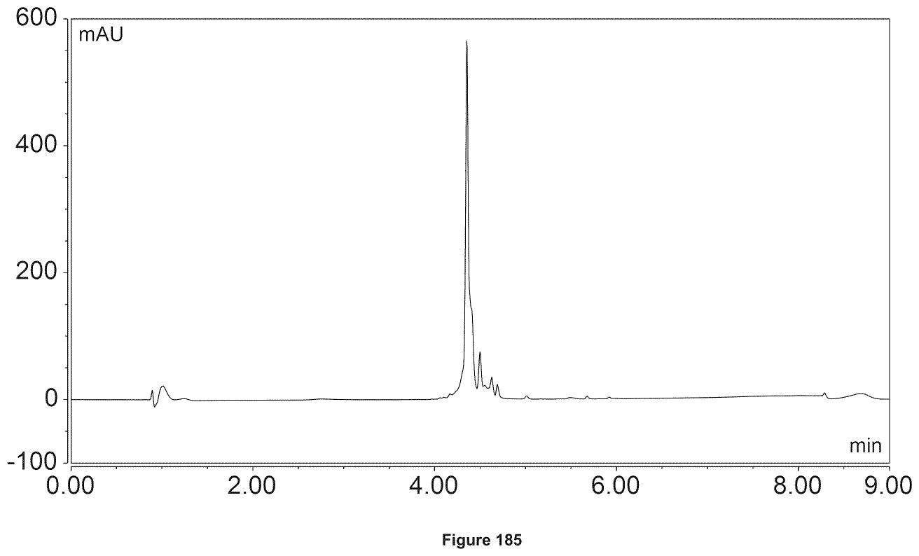

FIG. 185 shows an HPLC/UV chromatogram of the compound O—P5(PEG24)-amidopentyl-Phosphoramidate-N-(L-alanine-L-alanine-tert.-butylester)-O-Nampt-IN-1.

### O-5-(tert.-butoxy-carbonyl)-amidopentyl-Phosphoramidate-N-(L-alanine-L-alanine-tert.-butylester)-O-Paclitaxel

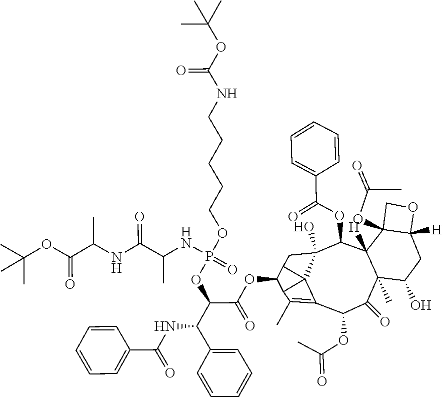

The title compound was synthesized in accordance with general Method 7 from 5 mg Paclitaxel (0.006 mmol). The product was obtained as white solid after preparative HPLC (Method C) and lyophilization. (4.6 mg, 0.0035 mmol, 58%). MS for C₆₇H₉₁N₄O₂₁P²⁺ [M+2H]²⁺ calcd.: 659.3 found 659.2.

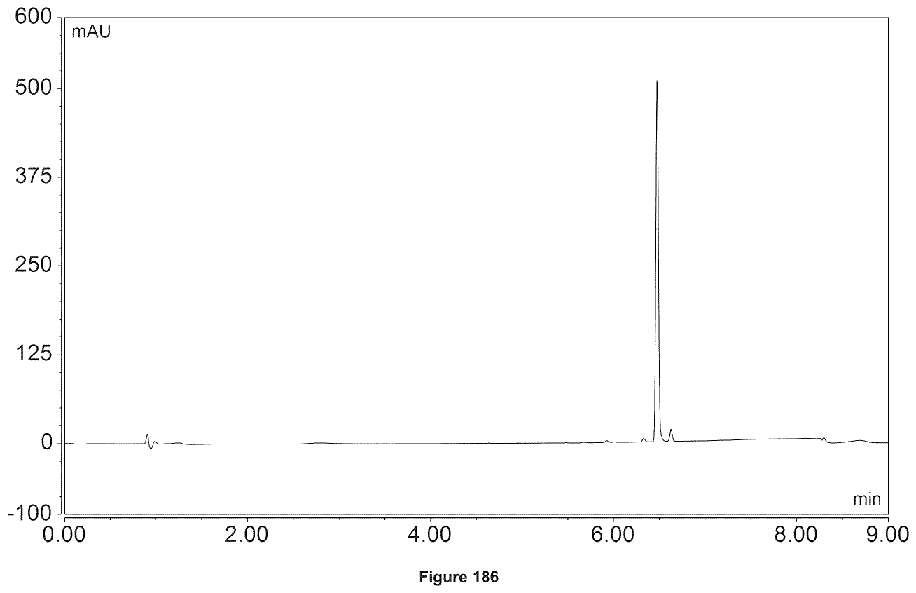

FIG. 186 shows an HPLC/UV chromatogram of the compound O-5-(tert.-butoxy-carbonyl)-amidopentyl-Phosphoramidate-N-(L-alanine-L-alanine-tert.-butylester)-O-Paclitaxel.

### O—P5(PEG24)-amidopentyl-Phosphoramidate-N-(L-alanine-L-alanine-tert.-butylester)-O-Paclitaxel

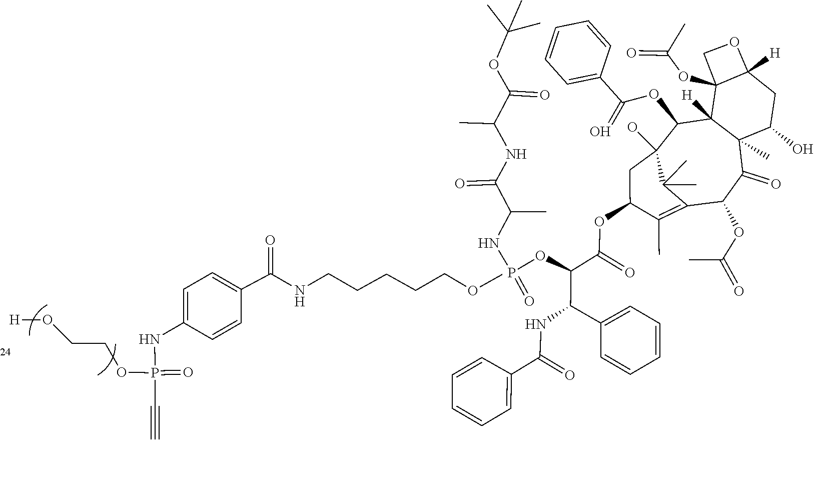

The title compound was synthesized in accordance with general Method 4 for Boc deprotection in the presence of the tert-butyl ester followed by amide coupling from 3.5 mg of O-5-(tert.-butoxy-carbonyl)-amidopentyl-Phosphoramidate-N-(L-alanine-L-alanine-tert.-butylester)-O-Paclitaxel (0.0027 mmol, 1 eq). The product was obtained as colorless oil after preparative HPLC (Method C) and lyophilization. (3.6 mg, 0.0015 mmol, 55% over two steps). HR-MS for C₁₁—H₁₈₆N₅O₄₆P₂³⁺ [M+3H]⁺ calcd.: 828.0621, found 828.0642.

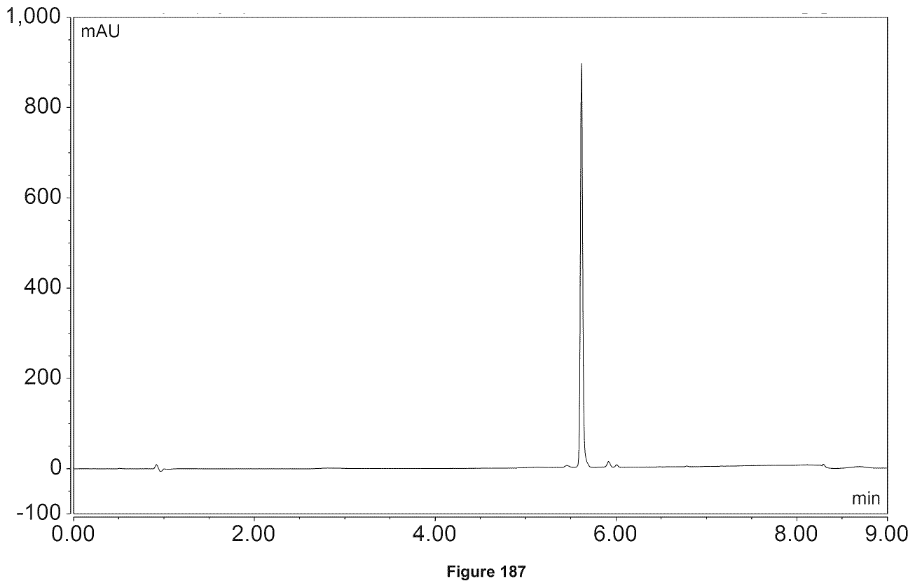

FIG. 187 shows an HPLC/UV chromatogram of the compound O—P5(PEG24)-amidopentyl-Phosphoramidate-N-(L-alanine-L-alanine-tert.-butylester)-O-Paclitaxel.

### O-5-(tert.-butoxy-carbonyl)-amidopentyl-Phosphoramidate-N-(L-alanine-L-alanine-tert.-butylester)-O-Triptolide

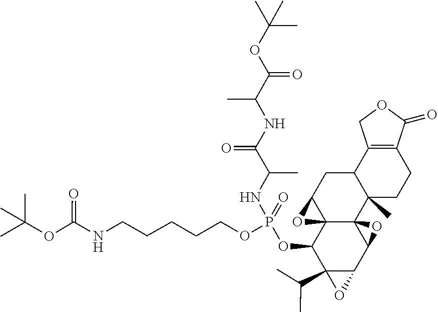 [SMILES: CC(NP(=O)(OCCCCCNC(=O)OC(C)(C)C)O[C@@H]1[C@@]2(C(C)C)O[C@H]2[C@@H]2O[C@]23[C@]12O[C@H]2CC1C2=C(CC[C@@]13C)C(=O)OC2)C(=O)NC(C)C(=O)OC(C)(C)C]

The title compound was synthesized in accordance with general Method 7 from 10 mg Triptolide (0.028 mmol). The product was obtained as white solid after preparative HPLC (Method C) and lyophilization. (19 mg, 0.023 mmol, 83%). MS for C₄₀H₆₃N₃O₁₃P⁺ [M+H]⁺ calcd.: 824.4 found 824.5.

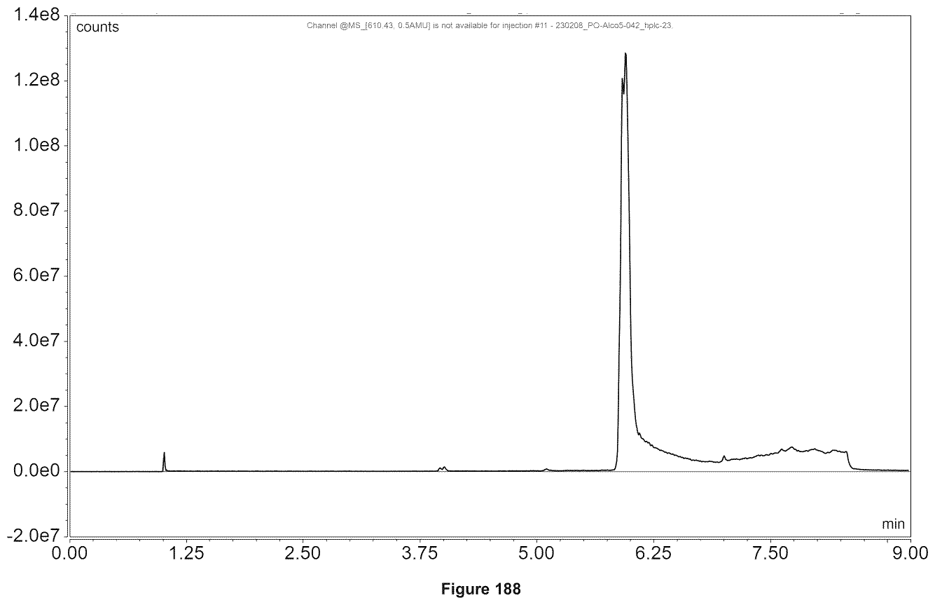

FIG. 188 shows an HPLC/UV chromatogram of the compound O-5-(tert.-butoxy-carbonyl)-amidopentyl-Phosphoramidate-N-(L-alanine-L-alanine-tert.-butylester)-O-Triptolide.

### O—P5(PEG24)-amidopentyl-Phosphoramidate-N-(L-alanine-L-alanine-tert.-butylester)-O-Triptolide

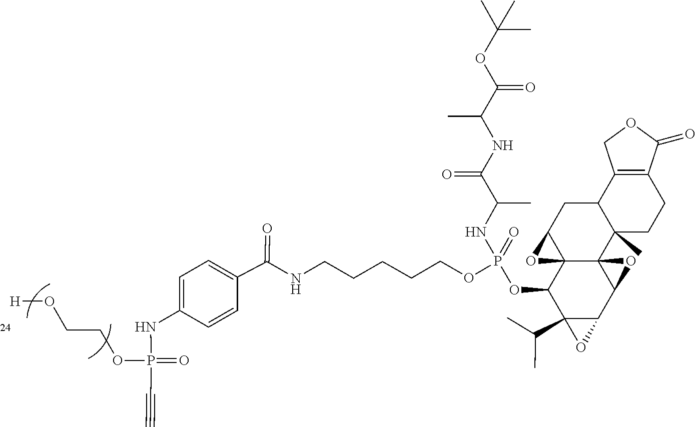

The title compound was synthesized in accordance with general Method 5 for Boc- and tert-butyl ester deprotection followed by amide coupling from 19 mg of O-(5-tert.-butoxy-carbonyl-aminopentyl)-N-(L-alanine-L-alanine-tert.-butylester)-O-(Triptolide)-Phosphoramidate (0.023 mmol, 1 eq). The product was obtained as colorless oil after preparative HPLC (Method C) and lyophilization. (10.5 mg, 0.005 mmol, 23% over two steps).

### O-(5-tert.-butoxy-carbonyl)-amidopentyl-Phosphoramidate-N-(alanine-tert.-butylester)-O—SN38

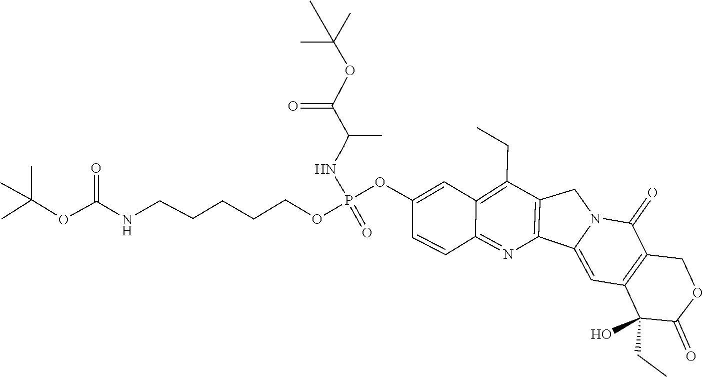

The title compound was synthesized in accordance with general Method 6 from 6.4 mg SN38 (0.016 mmol) and 21.7 mg O-(5-tert.-butoxy-carbonyl-amidopentyl)-Phosphoramidate-N-(alanine-tert.-butylester)-O-4-nitrophenyl (0.04 mmol). The product was obtained as white solid after preparative HPLC (Method C) and lyophilization. (9.9 mg, 0.013 mmol, 78%). HR-MS for C₃₉H₅₄N₄O₁₁P+ [M+H]+ calcd.: 785.3522, found 785.3574.

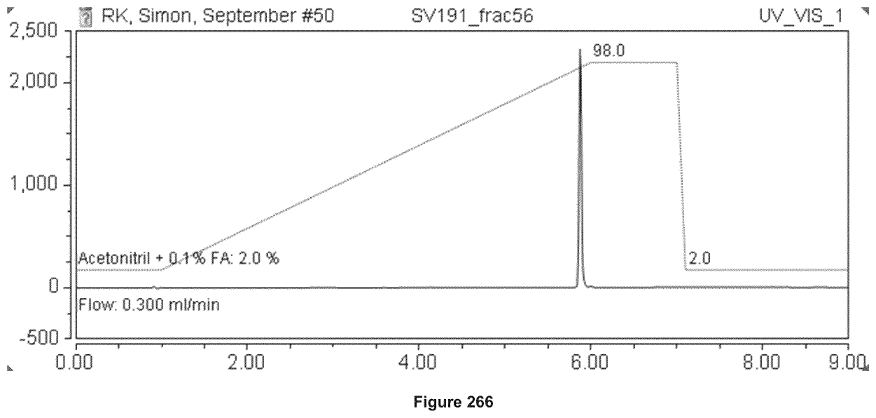

FIG. 266 shows an HPLC/UV chromatogram of the compound O-(5-tert.-butoxy-carbonyl)-amidopentyl-Phosphoramidate-N-(alanine-tert.-butylester)-O—SN38.

### O—P5(PEG24)-amidopentyl-Phosphoramidate-N-(L-alanine-tert.-butylester)-O-SN38

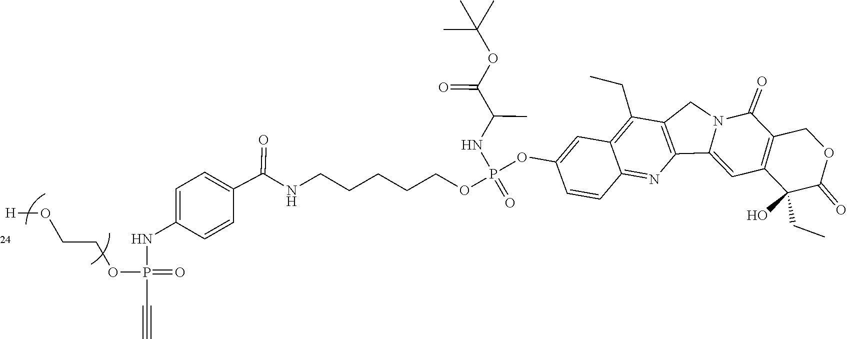

The title compound was synthesized in accordance to general method 4 for Boc deprotection in the presence of the tert-butyl ester followed by amide coupling from 10 mg of O-(5-tert.-butoxy-carbonyl-amidopentyl)-Phosphoramidate-N-(alanine-tert.-butylester)-O—SN38 (0.013 mmol, 1 eq). The product was obtained as yellowish oil after preparative HPLC (Method C) and lyophilization. (15.4 mg, 0.008 mmol, 61% over two steps). HR-MS for C₉₁H₁₄₉N₅O₃₆P₂²⁺ [M+2H]²⁺ calcd.: 974.9724, found 974.9752.

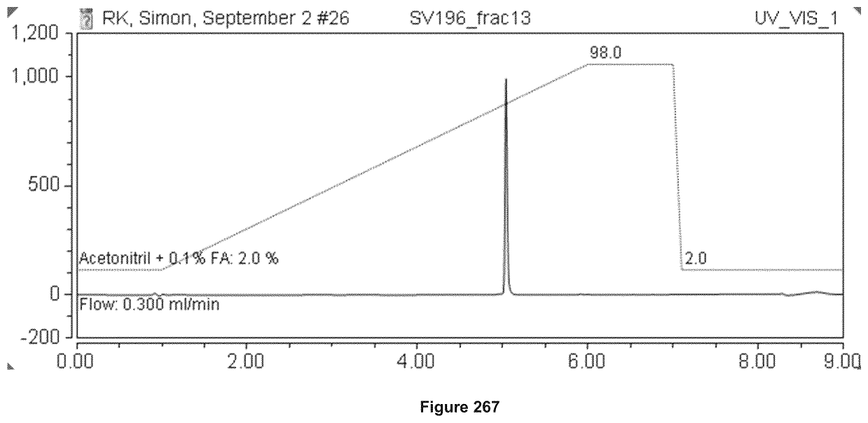

FIG. 267 shows an HPLC/UV chromatogram of the compound O—P5(PEG24)-amidopentyl-Phosphoramidate-N-(L-alanine-tert.-butylester)-O—SN38.

### O-(5-tert.-butoxy-carbonyl)-amidopentyl-Phosphoramidate-N-(2-ethoxy-6-(3R,4S,5S,6S)-(methoxycarbonyl)tetrahydro-2H-pyran-3,4,5-triyl triacetate)-O-4-nitrophenyl

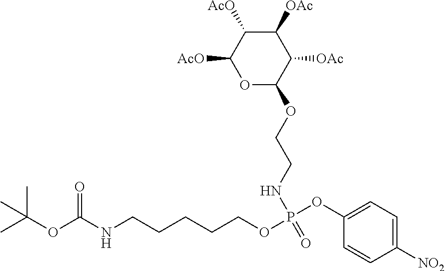

The title compound was synthesized in accordance with the general method 3 for the synthesis of nitrophenyl phosphoramidates from 135 mg 4-nitrophenyl phosphorodichloridate (0.5 mmol), 107 mg Boc-aminopentanol (0.5 mmol) and 199 mg 2-ethoxy-6-(3R,4S,5S,6S)-(methoxycarbonyl)tetrahydro-2H-pyran-3,4,5-triyl triacetate (0.5 mmol). The product was obtained as white solid (64 mg, 0.08 mmol, 16%). HR-MS for C₃₁H₄₇N₃O₁₇P⁺ [M+H]⁺ calcd.: 764.2638, found: 764.2619.

### O-(5-tert.-butoxy-carbonyl)-amidopentyl-Phosphoramidate-N-(2-ethoxy-6-(3R,4S,5S,6S)-(methoxycarbonyl)tetrahydro-2H-pyran-3,4,5-triyl triacetate)-O-SN38

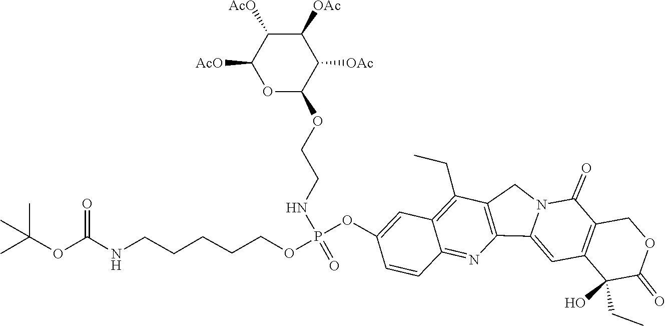 [SMILES: CCc1c2c(nc3ccc(OP(=O)(NCCO[C@H]4O[C@@H](OC(C)=O)[C@H](OC(C)=O)[C@@H](OC(C)=O)[C@@H]4OC(C)=O)OCCCCCNC(=O)OC(C)(C)C)cc13)-c1cc3c(c(=O)n1C2)COC(=O)[C@]3(O)CC]

The title compound was synthesized in accordance with general Method 6 from 5 mg SN38 (0.013 mmol) and 29 mg O-(5-tert.-butoxy-carbonyl)-amidopentyl-Phosphoramidate-N-(2-ethoxy-6-(3R,4S,5S,6S)-(methoxycarbonyl)tetrahydro-2H-pyran-3,4,5-triyl tri acetate)-O-4-nitro phenyl (0.038 mmol). The product was obtained as white solid after preparative HPLC (Method C) and lyophilization. (2.5 mg, 0.003 mmol, 23%). HR-MS for C₄₇H₆₂N₄O₁₉P⁺ [M+H]⁺ calcd.: 1017.3740, found 1017.3703.

### O—P5(PEG24)-amidopentyl-Phosphoramidate-N-(2-ethoxy-6-(3R,4S,5S,6S)-(methoxycarbonyl)tetrahydro-2H-pyran-3,4,5-triyl triacetate)-O-SN38

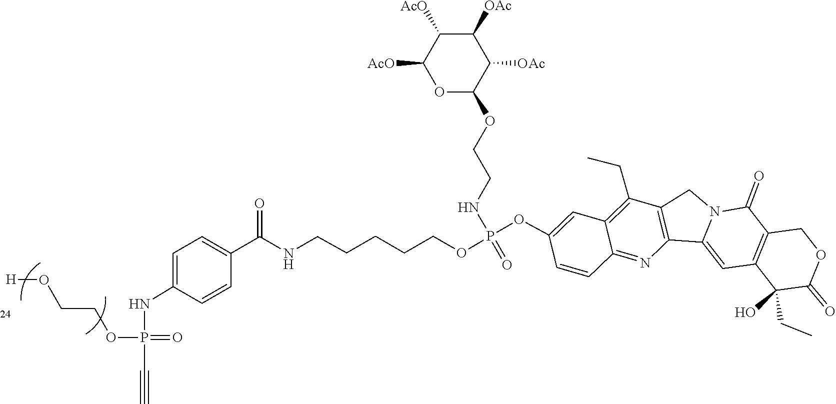

The title compound was synthesized in accordance to general method 4 for Boc-deprotection followed by amide coupling from 2.3 mg of O-(5-tert.-butoxy-carbonyl)-amidopentyl-Phosphoramidate-N-(2-ethoxy-6-(3R,4S,5S,6S)-(methoxycarbonyl)tetrahydro-2H-pyran-3,4,5-triyl triacetate)-O—SN38 (0.003 mmol, 1 eq). The product was obtained as yellowish oil after preparative HPLC (Method C) and lyophilization. (1.6 mg, 0.7 μmol, 23% over two steps). HR-MS for C₉₉H₁₅₇N₅O₄₄P₂²⁺ [M+2H]⁺ calcd.: 1091.4850, found 1091.4816.

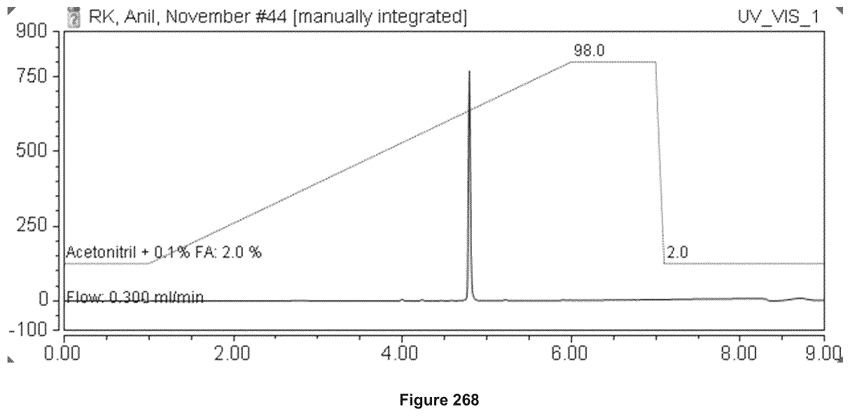

FIG. 268 shows an HPLC/UV chromatogram of the compound O—P5(PEG24)-amidopentyl-Phosphoramidate-N-(2-ethoxy-6-(3R,4S,5S,6S)-(methoxycarbonyl)tetrahydro-2H-pyran-3,4,5-triyl triacetate)-O—SN38.

### O-(5-tert.-butoxy-carbonyl)-amidopentyl-Phosphoramidate-N-(2-Acetoxy-ethyl)-O-4-nitrophenyl

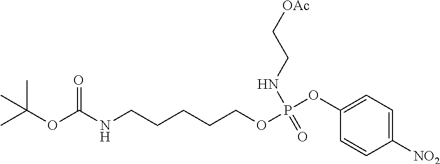 [SMILES: CC(=O)OCCNP(=O)(OCCCCCNC(=O)OC(C)(C)C)Oc1ccc([N+](=O)[O-])cc1]

The title compound was synthesized in accordance with the general method 3 for the synthesis of nitrophenyl phosphoramidates from 250 mg 4-nitrophenyl phosphorodichloridate (1 mmol), 198 mg Boc-aminopentanol (1 mmol) and 136 mg 2-Acetoxy-ethylamine (1 mmol). The product was obtained as white solid (157 mg, 0.32 mmol, 32%). HR-MS for C₂₀H₃₃N₃O₉P⁺ [M+Na]+ calcd.: 490.1949512.2, found 512.1.

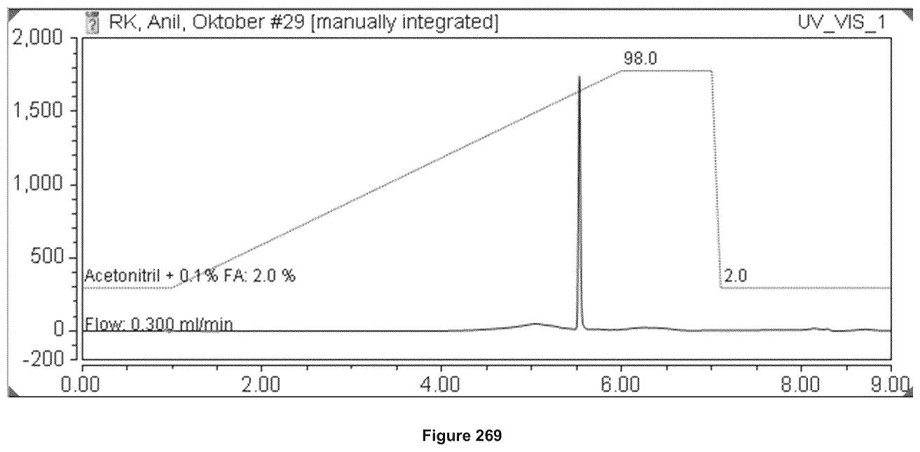

FIG. 269 shows an HPLC/UV chromatogram of the compound O-(5-tert.-butoxy-carbonyl)-amidopentyl-Phosphoramidate-N-(2-Acetoxy-ethyl)-O-4-nitrophenyl.

### O-(5-tert.-butoxy-carbonyl)-amidopentyl-Phosphoramidate-N-(2-Acetoxy-ethyl)-O-DXD

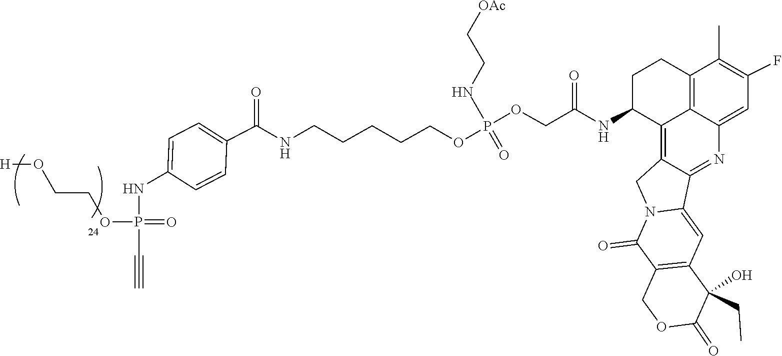

The title compound was synthesized in accordance with general Method 6 from 5 mg DXD (0.010 mmol) and 15 mg O-(5-tert.-butoxy-carbonyl)-amidopentyl-Phosphoramidate-N-(2-Acetoxy-ethyl)-O-4-nitrophenyl (0.038 mmol). The product was obtained as white solid after preparative HPLC (Method C) and lyophilization. (3.6 mg, 0.005 mmol, 50%). HR-MS for C₄₀H₅₂FN₅O₁₂P⁺ [M+H]⁺ calcd.: 844.3329 found 844.3329.

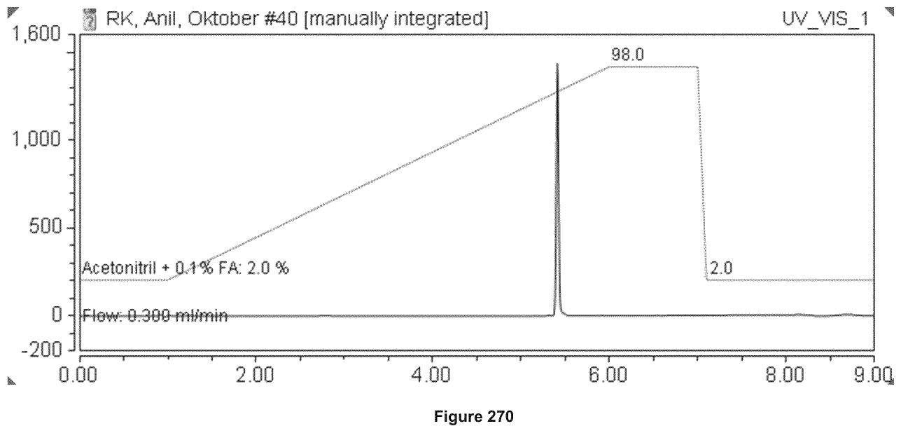

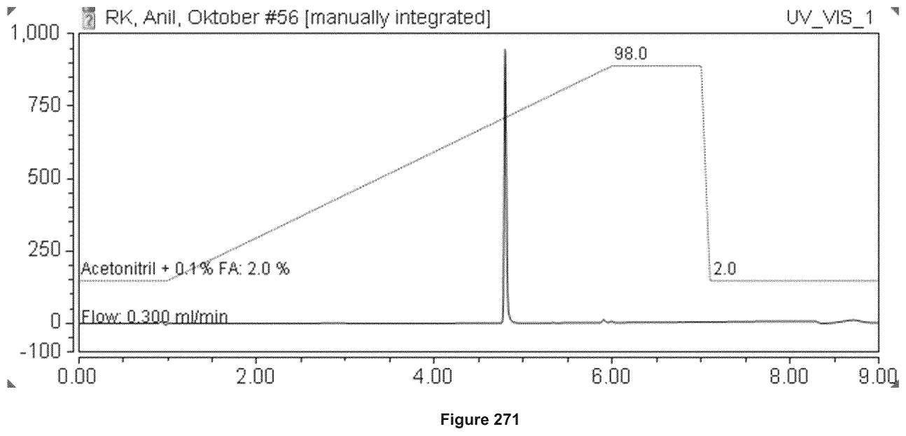

FIG. 270 shows an HPLC/UV chromatogram of the compound O-(5-tert.-butoxy-carbonyl)-amidopentyl-Phosphoramidate-N-(2-Acetoxy-ethyl)-O-DXD.

### O—P5(PEG24)-amidopentyl-Phosphoramidate-N-(2-Acetoxy-ethyl)-O-DXD

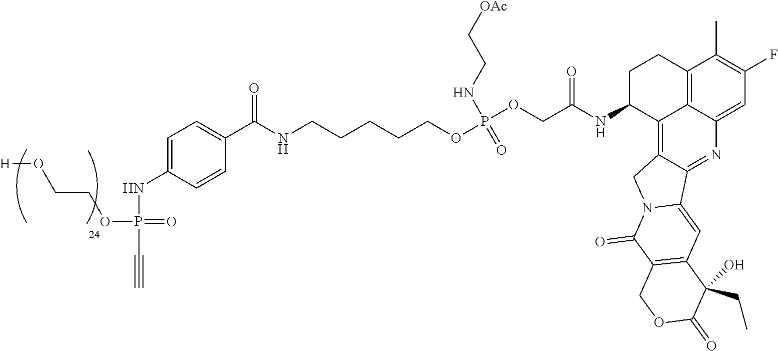

The title compound was synthesized in accordance to general method 5 for Boc-deprotection followed by amide coupling from 3.6 mg of O-(5-tert.-butoxy-carbonyl)-amidopentyl-Phosphoramidate-N-(2-Acetoxy-ethyl)-O-DXD (0.005 mmol, 1 eq). The product was obtained as yellowish oil after preparative HPLC (Method C) and lyophilization. (6.3 mg, 0.003 mmol, 62% over two steps). HR-MS for C₉₂H₁₄₇FN₆O₃₇P₂²⁺ [M+2H]⁺ calcd.: 1004.4627, found 1004.4586.

FIG. 271 shows an HPLC/UV chromatogram of the compound O—P5(PEG24)-amidopentyl-Phosphoramidate-N-(2-Acetoxy-ethyl)-O-DXD.

### O-(5-tert.-butoxy-carbonyl)-amidopentyl-Phosphoramidate-N-(2-Acetamido-ethyl)-O-4-nitrophenyl

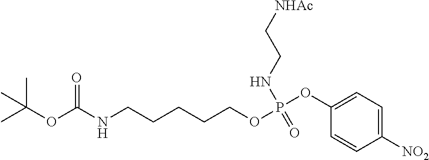 [SMILES: CC(=O)NCCNP(=O)(OCCCCCNC(=O)OC(C)(C)C)Oc1ccc([N+](=O)[O-])cc1]

The title compound was synthesized in accordance with the general method 3 for the synthesis of nitrophenyl phosphoramidates from 403 mg 4-nitrophenyl phosphorodichloridate (1.6 mmol), 305 mg Boc-aminopentanol (1.5 mmol) and 169 mg N-(2-aminoethyl)acetamide (1.7 mmol). The product was obtained as white solid (653 mg, 1.32 mmol, 89%). HR-MS for C₂₀H₃₄N₄O₈P⁺ [M+H]⁺ calcd.: 489.2109, found 489.15.

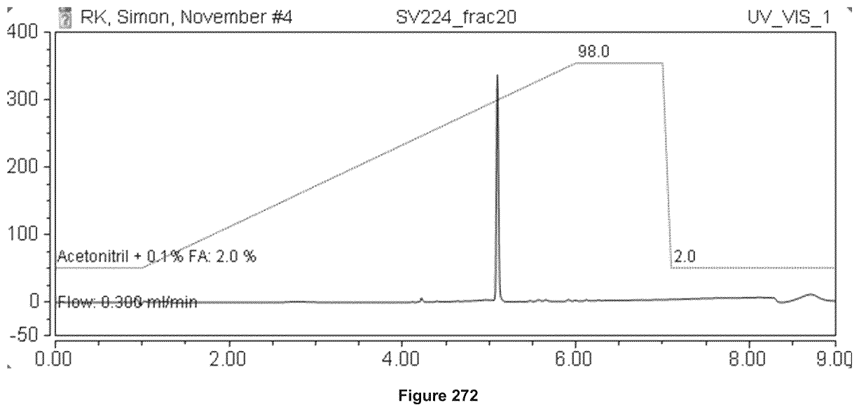

FIG. 272 shows an HPLC/UV chromatogram of the compound O-(5-tert.-butoxy-carbonyl)-amidopentyl-Phosphoramidate-N-(2-Acetamido-ethyl)-O-4-nitrophenyl.

### O-(5-tert.-butoxy-carbonyl)-amidopentyl-Phosphoramidate-N-(2-Acetamido-ethyl)-O-SN38

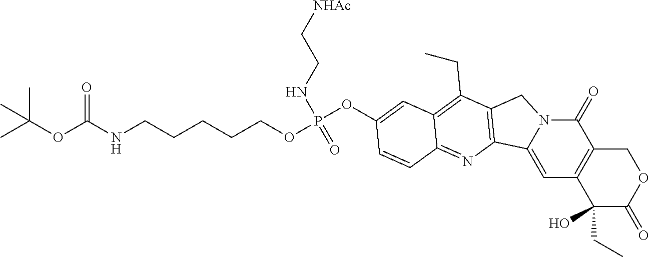 [SMILES: CCc1c2c(nc3ccc(OP(=O)(NCCNC(C)=O)OCCCCCNC(=O)OC(C)(C)C)cc13)-c1cc3c(c(=O)n1C2)COC(=O)[C@]3(O)CC]

The title compound was synthesized in accordance with general Method 6 from 5.5 mg SN38 (0.014 mmol) and 17.5 mg O-(5-tert.-butoxy-carbonyl)-amidopentyl-Phosphoramidate-N-(2-Acetamido-ethyl)-O-4-nitrophenyl (0.04 mmol). The product was obtained as white solid after preparative HPLC (Method C) and lyophilization. (5.7 mg, 0.008 mmol, 54%). HR-MS for C₃₆H₄₉N₅O₁₀P⁺ [M+H]⁺ calcd.: 742.3212 found 742.3198.

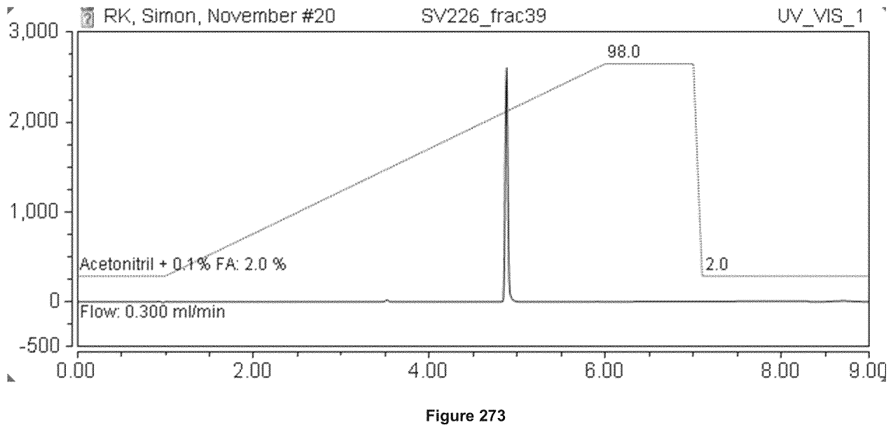

FIG. 273 shows an HPLC/UV chromatogram of the compound O-(5-tert.-butoxy-carbonyl)-amidopentyl-Phosphoramidate-N-(2-Acetamido-ethyl)-O—SN38.

### O-P5-(PEG24)-amidopentyl-Phosphoramidate-N-(2-Acetamido-ethyl)-O-SN38

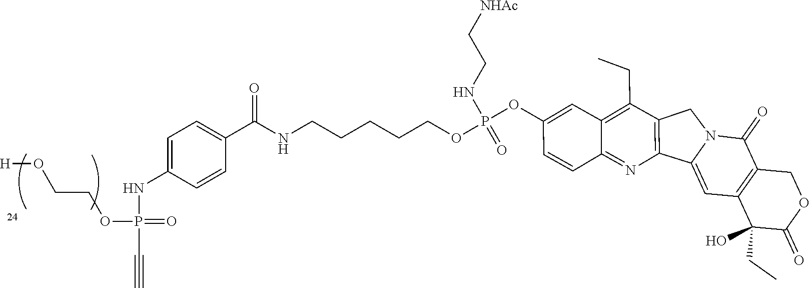

The title compound was synthesized in accordance to general method for Boc-deprotection followed by amide coupling from 5.7 mg O-(5-tert.-butoxy-carbonyl)-amidopentyl-Phosphoramidate-N-(2-Acetamido-ethyl)-O—SN38 (0.008 mmol, 1 eq). The product was obtained as yellowish oil after preparative HPLC (Method C) and lyophilization. (10.9 mg, 0.006 mmol, 75% over two steps). HR-MS for C88H144N6O35P22+[M+2H]+ calcd.: 953.4569, found 953.4541.

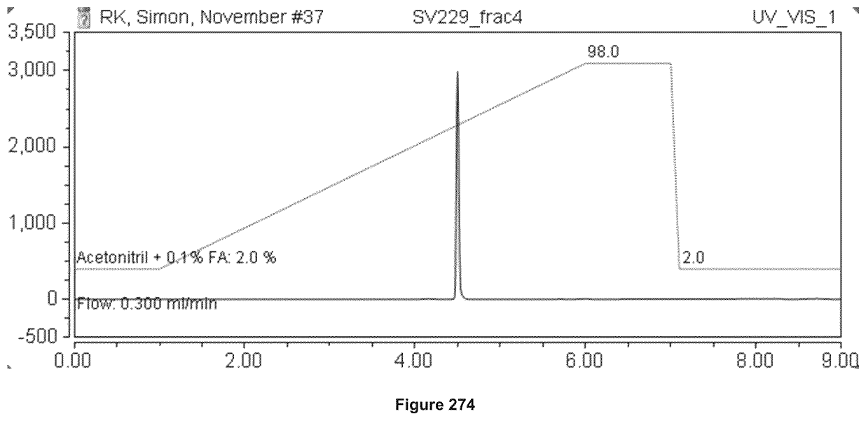

FIG. 274 shows an HPLC/UV chromatogram of the compound O-P5-(PEG24)-amidopentyl-Phosphoramidate-N-(2-Acetamido-ethyl)-O—SN38.

### O-5-(phenylmethoxy-carbonyl)-amidopentyl-Phosphoramidate-N-(2-2-Diethoxy-ethyl)-O—SN38

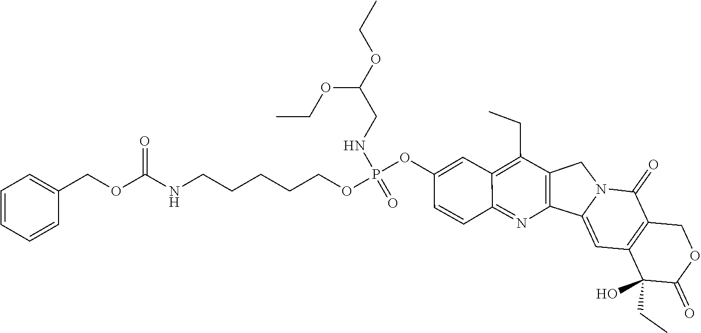 [SMILES: CCOC(CNP(=O)(OCCCCCNC(=O)OCc1ccccc1)Oc1ccc2nc3c(c(CC)c2c1)Cn1c-3cc2c(c1=O)COC(=O)[C@]2(O)CC)OCC]

The title compound was synthesized in accordance with general Method 1 from 4.6 mg SN38 (0.012 mmol) and 32.5 mg O-5-(phenylmethoxy-carbonyl)-amidopentyl-Phosphoramidate-N-(2-2-Diethoxy-ethyl)-O-4-nitrophenyl (0.059 mmol). The product was obtained as yellow solid after preparative HPLC (Method C) and lyophilization. (3.5 mg, 0.004 mmol, 36%). HR-MS for C41 H52N4O11P+ [M+H]+ calcd.: 807.3365 found 807.3366.

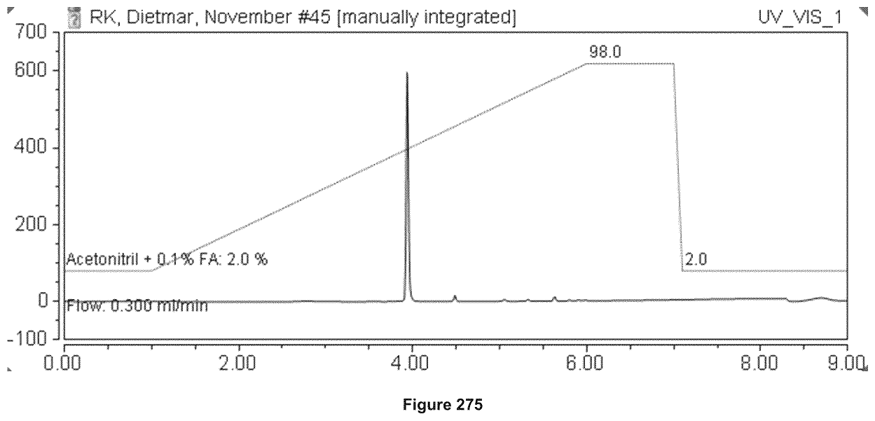

FIG. 275 shows an HPLC/UV chromatogram of the compound O-5-(phenylmethoxy-carbonyl)-amidopentyl-Phosphoramidate-N-(2-2-Diethoxy-ethyl)-O—SN38.

### O—P5(PEG24)-amidopentyl-Phosphoramidate-N-(2-2-Diethoxy-ethyl)-O-SN38

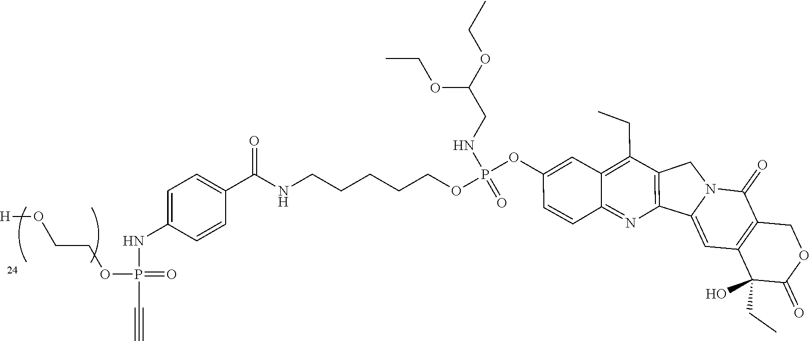

The title compound was synthesized from 7.3 mg of O-5-(phenylmethoxy-carbonyl)-amidopentyl-Phosphoramidate-N-(2-2-Diethoxy-ethyl)-O—SN38 (0.008 mmol, 1 eq), which was dissolved in 1 mL MeOH and 1 mg Pd/C and stirred under H2 atmosphere to remove the Cbz protecting group. After complete deprotection the reaction was filtered, and the solvent was removed under reduced pressure. The residue was dissolved in 0.5 ml DMSO. 1.5 eq. PyBop, dissolved in DMSO and 5.0 eq. DIPEA were added. After 5 minutes, 1.0 eq of P5-PEG24-COOH dissolved in DMSO was added and the mixture was stirred at RT for 1h. The mixture was diluted with H2O/MeCN (2:1) and directly subjected to preparative HPLC purification. The product was obtained as yellowish oil after preparative HPLC (Method C) and lyophilization. (8.2 mg, 0.004 mmol, 50% over two steps). HR-MS for C90H149N5O36P22+[M+2H]+ calcd.: 968.9724, found 968.9790.

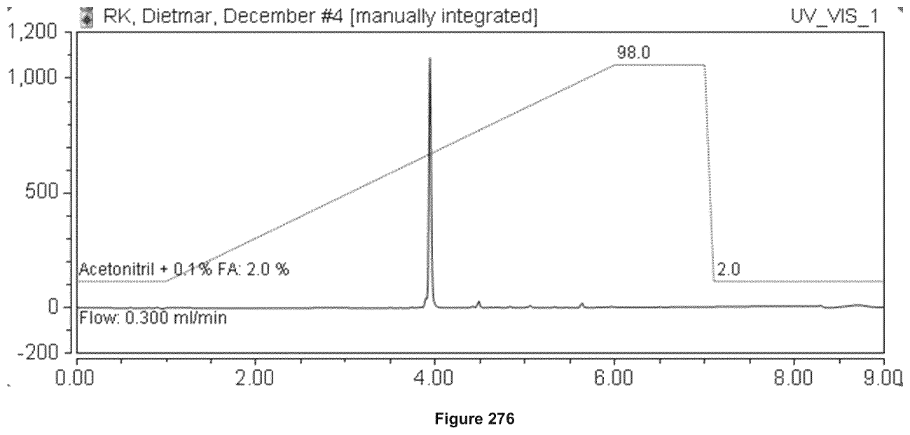

FIG. 276 shows an HPLC/UV chromatogram of the compound O—P5(PEG24)-amidopentyl-Phosphoramidate-N-(2-2-Diethoxy-ethyl)-O—SN38.

### O-(5-tert.-butoxy-carbonyl)-amidopentyl-Phosphoramidate-O-(lactic acid-iso-propylester)-O-4-nitrophenyl

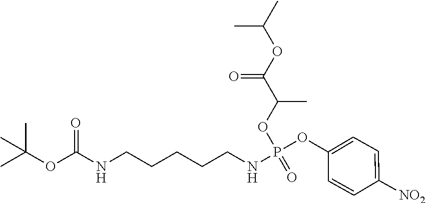 [SMILES: CC(C)OC(=O)C(C)OP(=O)(NCCCCCNC(=O)OC(C)(C)C)Oc1ccc([N+](=O)[O-])cc1]

The title compound was synthesized in accordance with the general method 3 for the synthesis of nitrophenyl phosphoramidates from 406 mg 4-nitrophenyl phosphorodichloridate (1.6 mmol), 336 mg N-Boc-1,5-diaminopentane (1.66 mmol) and 200 mg isopropyl lactate (1.5 mmol). The product was obtained as white solid (510 mg, 0.98 mmol, 65%). MS for C₂₂H₃₇N₃O₉P⁺ [M+H]⁺ calcd.: 518.23, found 518.21.

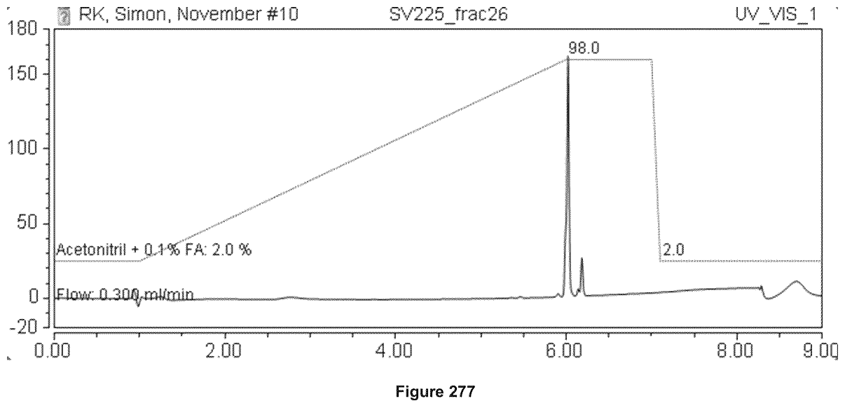

FIG. 277 shows an HPLC/UV chromatogram of the compound O-(5-tert.-butoxy-carbonyl)-amidopentyl-Phosphoramidate-O-(lactic acid-iso-propylester)-O-4-nitrophenyl.

### O-(5-tert.-butoxy-carbonyl)-amidopentyl-Phosphoramidate-O-(lactic acid-iso-propylester)-O-SN38

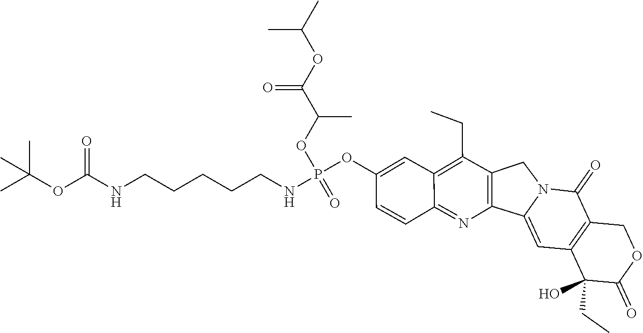 [SMILES: CCc1c2c(nc3ccc(OP(=O)(NCCCCCNC(=O)OC(C)(C)C)OC(C)C(=O)OC(C)C)cc13)-c1cc3c(c(=O)n1C2)COC(=O)[C@]3(O)CC]

The title compound was synthesized in accordance with general Method 6 from 8.4 mg SN38 (0.021 mmol) and 33.2 mg O-(5-tert.-butoxy-carbonyl)-amidopentyl-Phosphoramidate-O-(lactic acid-iso-propylester)-O-4-nitrophenyl (0.06 mmol). The product was obtained as white solid after preparative HPLC (Method C) and lyophilization. (11.3 mg, 0.015 mmol, 69%). HR-MS for C₃₈H₅₂N₄O₁₁P⁺ [M+H]⁺ calcd.: 771.3365 found 771.3380.

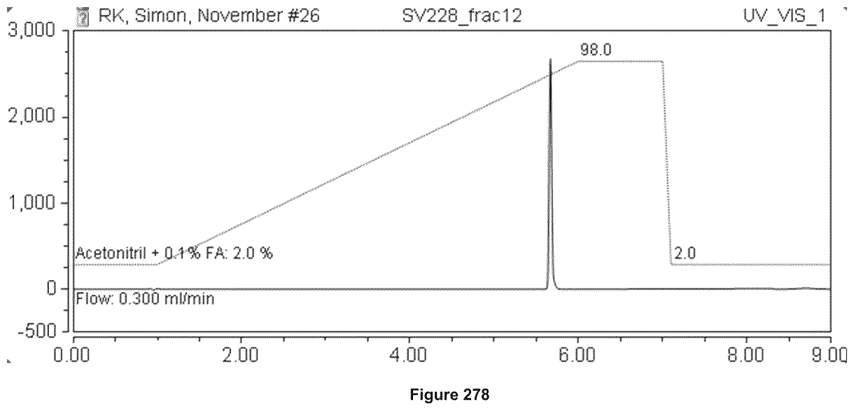

FIG. 278 shows an HPLC/UV chromatogram of the compound O-(5-tert.-butoxy-carbonyl)-amidopentyl-Phosphoramidate-O-(lactic acid-iso-propylester)-O—SN38.

### O-P5-(PEG24)-amidopentyl-Phosphoramidate-O-(lactic acid-iso-propylester)-O-SN38

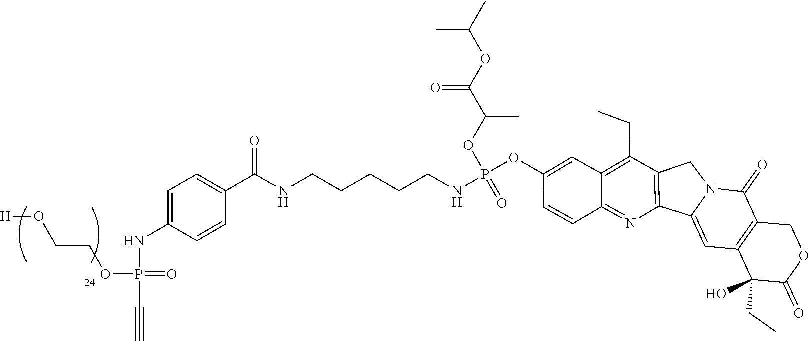

The title compound was synthesized in accordance to general method 4 for Boc-deprotection followed by amide coupling from 11.3 mg O-(5-tert.-butoxy-carbonyl)-amidopentyl-Phosphoramidate-O-(lactic acid-iso-propylester)-O—SN38 (0.015 mmol, 1 eq). The product was obtained as yellowish oil after preparative HPLC (Method C) and lyophilization. (25.2 mg, 0.013 mmol, 86% over two steps). HR-MS for C₉₀H₁₄₇N₅O₃₆P₂²⁺ [M+2H]⁺ calcd.: 967.9645, found 967.9618.

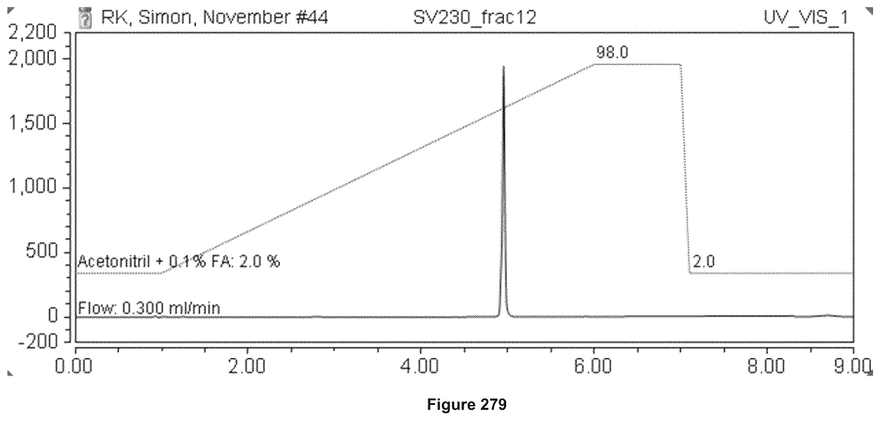

FIG. 279 shows an HPLC/UV chromatogram of the compound O-P5-(PEG24)-amidopentyl-Phosphoramidate-O-(lactic acid-iso-propylester)-O—SN38.

### O-(5-tert.-butoxy-carbonyl)-amidopentyl-Phosphoramidate-O-(glycolic acid-iso-propylester)-N-Exatecan

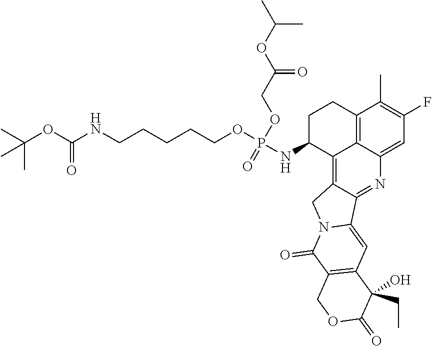 [SMILES: CC[C@@]1(O)C(=O)OCc2c1cc1n(c2=O)Cc2c-1nc1cc(F)c(C)c3c1c2[C@@H](NP(=O)(OCCCCCNC(=O)OC(C)(C)C)OCC(=O)OC(C)C)CC3]

The title compound was synthesized in accordance with general Method 8 from 580 mg boc-aminopentane phenyl phosphite (1.7 mmol) and 200 mg isopropyl glycolate (1.7 mmol), which yielded 153 mg of boc-aminopentane-isopropyl glycol-phosphite. The title compound was synthesized from 13.9 mg (0.04 mmol) of the phosphite and 13.4 mg Exatecan (0.025 mmol). The product was obtained as yellow solid after preparative HPLC (Method C) and lyophilization. (14.9 mg, 0.019 mmol, 76%). HR-MS for C₃₉H₅₁FN₄O₁₁P⁺ [M+H]⁺ calcd.: 801.3271 found 801.3237.

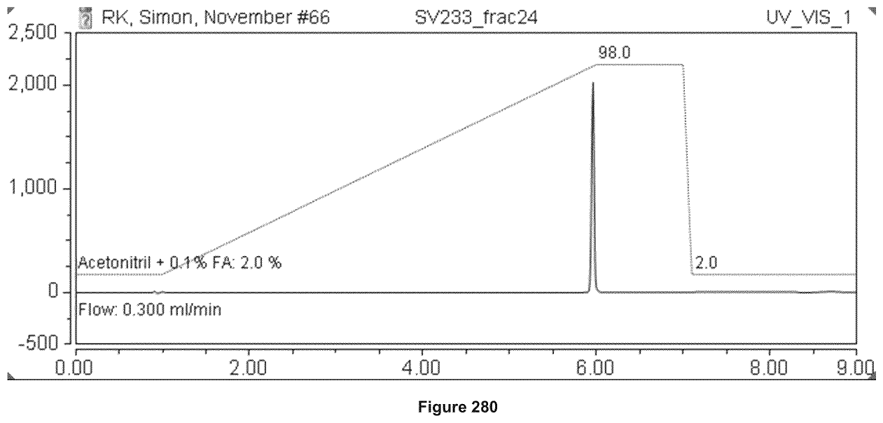

FIG. 280 shows an HPLC/UV chromatogram of the compound O-(5-tert.-butoxy-carbonyl)-amidopentyl-Phosphoramidate-O-(glycolic acid-iso-propylester)-N-Exatecan.

### O-P5-(PEG24)-amidopentyl-Phosphoramidate-O-(glycolic acid-iso-propylester)-N-Exatecan

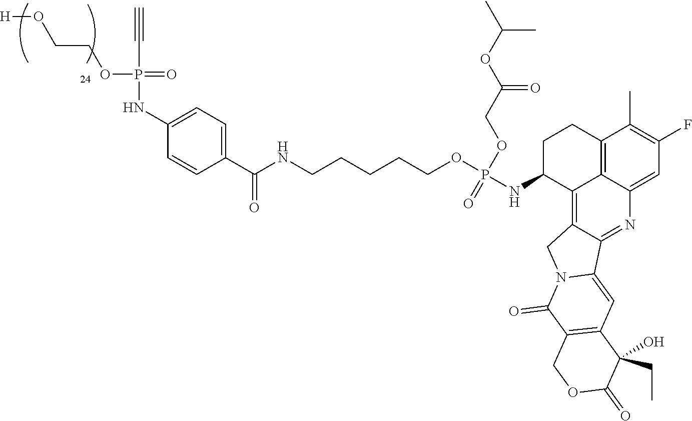

The title compound was synthesized in accordance to general method 4 for Boc-deprotection followed by amide coupling from 14.9 mg O-(5-tert.-butoxy-carbonyl)-amidopentyl-Phosphoramidate-O-(glycolic acid-iso-propylester)-N-Exatecan (0.019 mmol, 1 eq). The product was obtained as yellowish oil after preparative HPLC (Method C) and lyophilization. (29.3 mg, 0.015 mmol, 79% over two steps). HR-MS for C₉₁H₁₄₆FN₅O₃₆P₂²⁺ [M+2H]⁺ calcd.: 982.9598, found 982.9562.

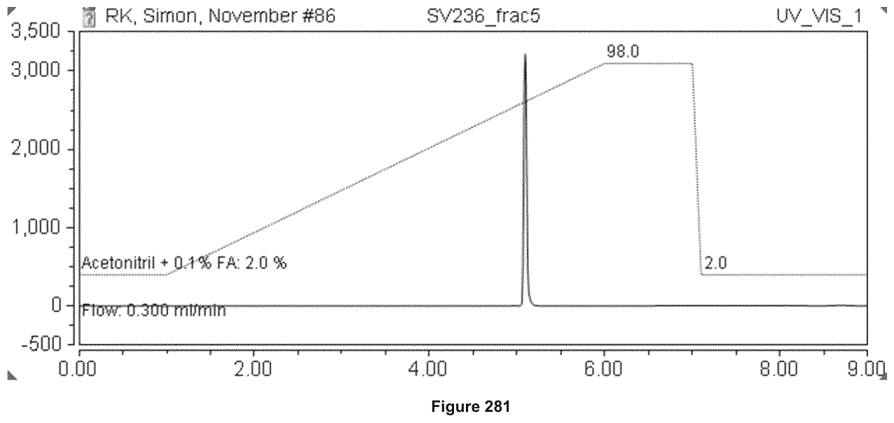

FIG. 281 shows an HPLC/UV chromatogram of the compound O-P5-(PEG24)-amidopentyl-Phosphoramidate-O-(glycolic acid-iso-propylester)-N-Exatecan.

### O-(5-tert.-butoxy-carbonyl)-amidopentyl-Phosphoramidate-N-(L-alanine-L-alanine-tert.-butyl ester)-O-BAY-2402234

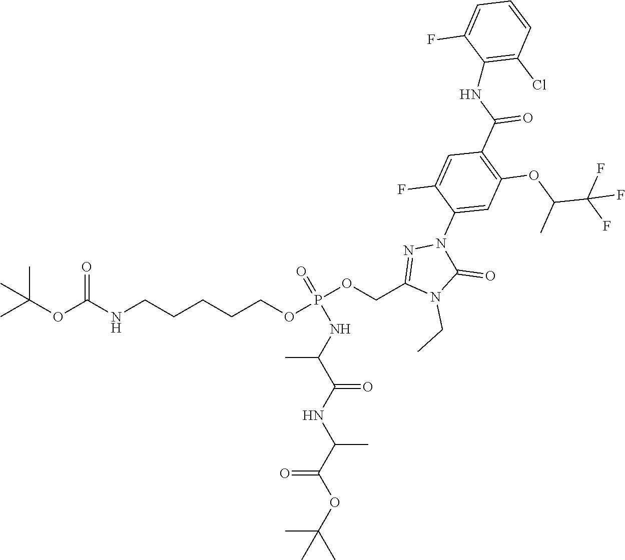 [SMILES: CCn1c(COP(=O)(NC(C)C(=O)NC(C)C(=O)OC(C)(C)C)OCCCCCNC(=O)OC(C)(C)C)nn(-c2cc(OC(C)C(F)(F)F)c(C(=O)Nc3c(F)cccc3Cl)cc2F)c1=O]

The title compound was synthesized in accordance with general Method 6 from 2 mg BAY-2402234 (0.004 mmol) and 11.6 mg O-(5-tert.-butoxy-carbonyl)-amidopentyl-Phosphoramidate-O-(L-alanine-L-alanine-tert.-butyl ester)-O-4-nitrophenyl (0.019 mmol). The product was obtained as white solid after preparative HPLC (Method C) and lyophilization. (2.6 mg, 0.003 mmol, 70%). HR-MS for C₄₁H₅₇ClF₅N₇O₁₁P⁺ [M+H]⁺ calcd.: 984.3457, found 984.34158.

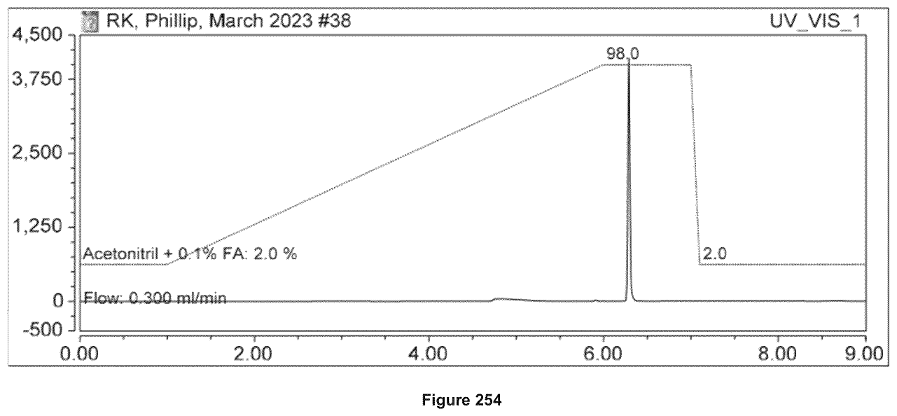

FIG. 254 shows an HPLC/UV chromatogram of the compound O-(5-tert.-butoxy-carbonyl)-amidopentyl-Phosphoramidate-N-(L-alanine-L-alanine-tert.-butyl ester)-O-BAY-2402234.

### O-P5-(PEG24)-amidopentyl-Phosphoramidate-N-(L-alanine-L-alanine)-O-BAY-2402234

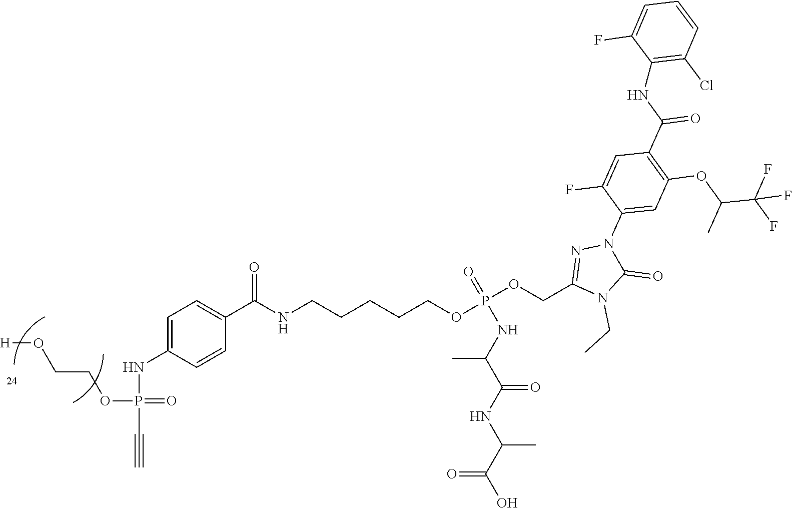

The title compound was synthesized in accordance to general method 5 for Boc-deprotection followed by amide coupling from 2.13 mg O-(5-tert.-butoxy-carbonyl)-N-(L-alanine-L-alanine-tert.-butyl ester)-O-(BAY-2402234)-Phosphoramidate (0.003 mmol, 1 eq). The product was obtained as yellowish oil after preparative HPLC (Method C) and lyophilization. (2.2 mg, 0.001 mmol, 48% over two steps). HR-MS for C₈₉H₁₄₄ClF₅N₈O₃₆P₂²⁺ [M+2H]⁺ calcd.: 1046.4378, found 1046.43863.

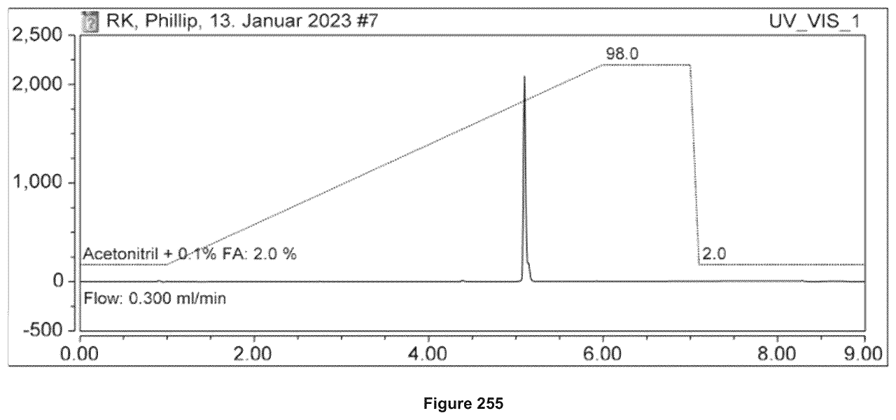

FIG. 255 shows an HPLC/UV chromatogram of the compound O-P5-(PEG24)-amidopentyl-Phosphoramidate-N-(L-alanine-L-alanine)-O-BAY-2402234.

### O-(5-tert-butoxy-carbonyl)-amidopentyl-Phosphoramidate-N-(L-alanine-tert.-butyl ester)-O—ON-013100

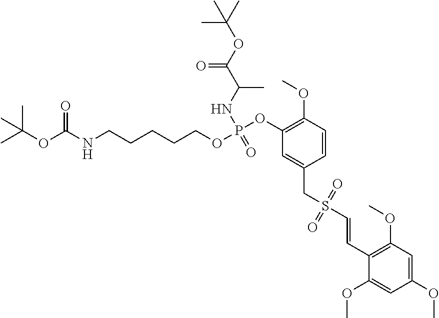 [SMILES: COc1cc(OC)c(/C=C/S(=O)(=O)Cc2ccc(OC)c(OP(=O)(NC(C)C(=O)OC(C)(C)C)OCCCCCNC(=O)OC(C)(C)C)c2)c(OC)c1]

The title compound was synthesized in accordance with general Method 6 from 5 mg ON-013100 (0.013 mmol) and 20.2 mg O-(5-tert.-butoxy-carbonyl)-amidopentyl-Phosphoramidate-O-(alanine-tert.-butyl ester)-O-4-nitrophenyl (0.04 mmol). The product was obtained as white solid after preparative HPLC (Method C) and lyophilization. (8.6 mg, 0.011 mmol, 84%). MS for C₃₆H₅₆N₂O₁₃PS⁺ [M+H]⁺ calcd.: 787.3235 found 787.3.

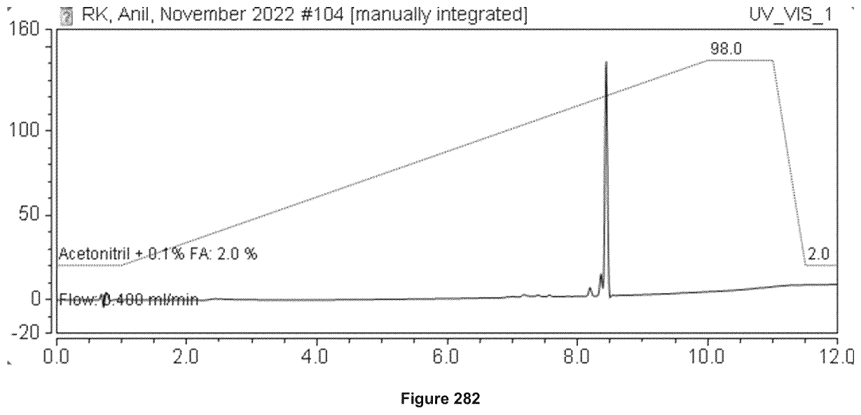

FIG. 282 shows an HPLC/UV chromatogram of the compound O-(5-tert.-butoxy-carbonyl)-amidopentyl-Phosphoramidate-N-(L-alanine-tert.-butyl ester)-O—ON-013100.

### O—P5(PEG24)-amidopentyl-Phosphoramidate-N-(L-alanine-tert.-butyl ester)-O—ON-013100

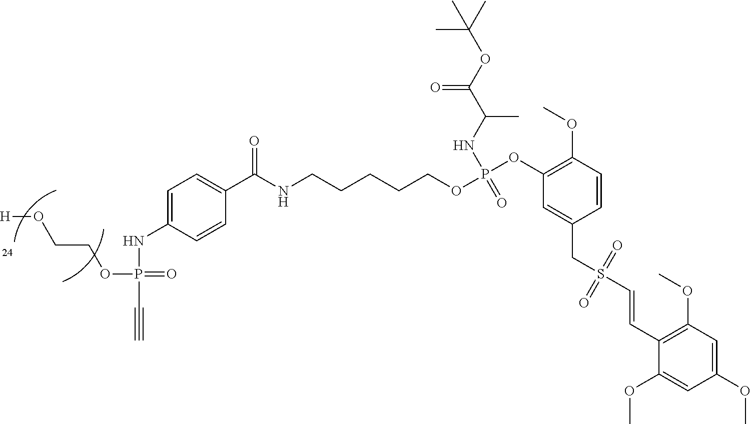

The title compound was synthesized in accordance to general method 4 for Boc- and tert-butyl deprotection followed by amide coupling from 11.8 mg of O-(5-tert.-butoxy-carbonyl-aminopentyl)-N-(L-alanine-tert.-butyl ester)-O—(ON-013100)-Phosphoramidate (0.015 mmol, 1 eq). The product was obtained as yellowish oil after preparative HPLC (Method C) and lyophilization. (12.3 mg, 0.006 mmol, 42% over two steps). MS for C₈₈H₁₅₁N₃O₃₈P₂S²⁺[M+2H]⁺ calcd.: 975.96, found 976.0.

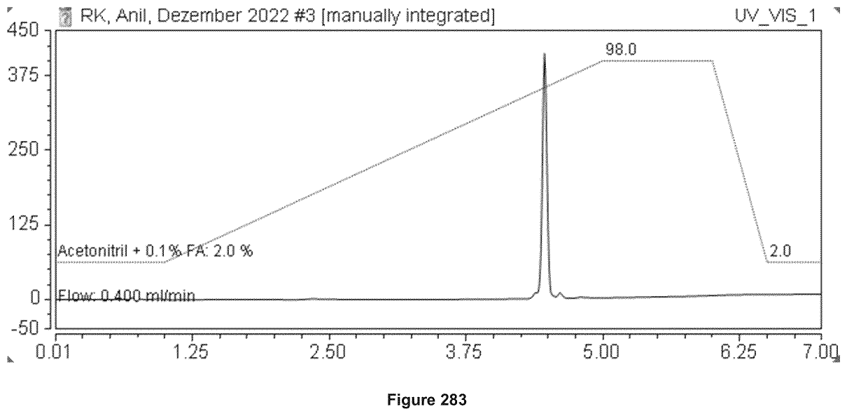

FIG. 283 shows an HPLC/UV chromatogram of the compound O—P5(PEG24)-amidopentyl-Phosphoramidate-N-(L-alanine-tert.-butyl ester)-O—ON-013100.

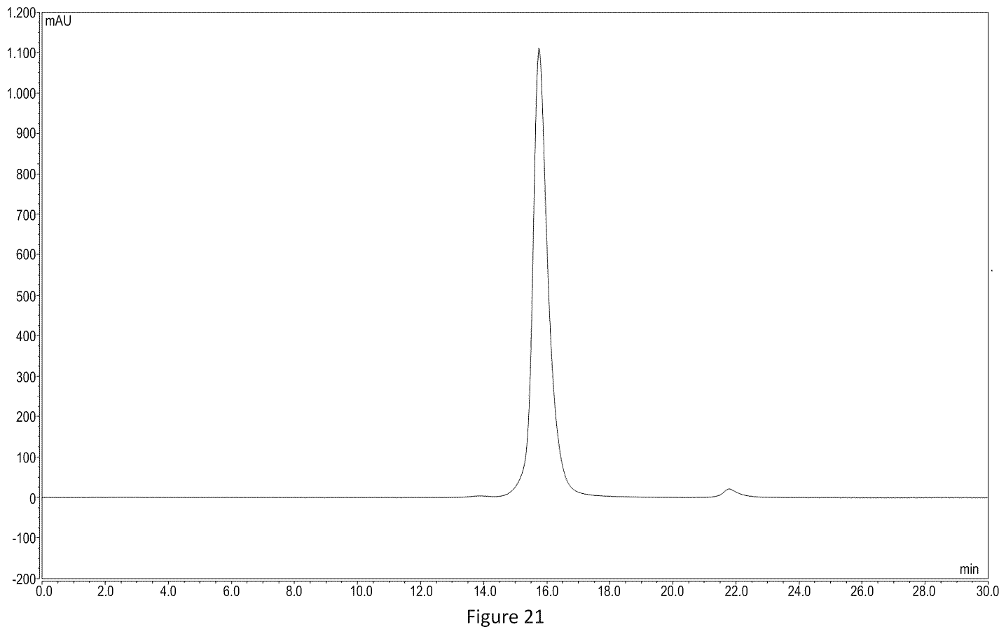

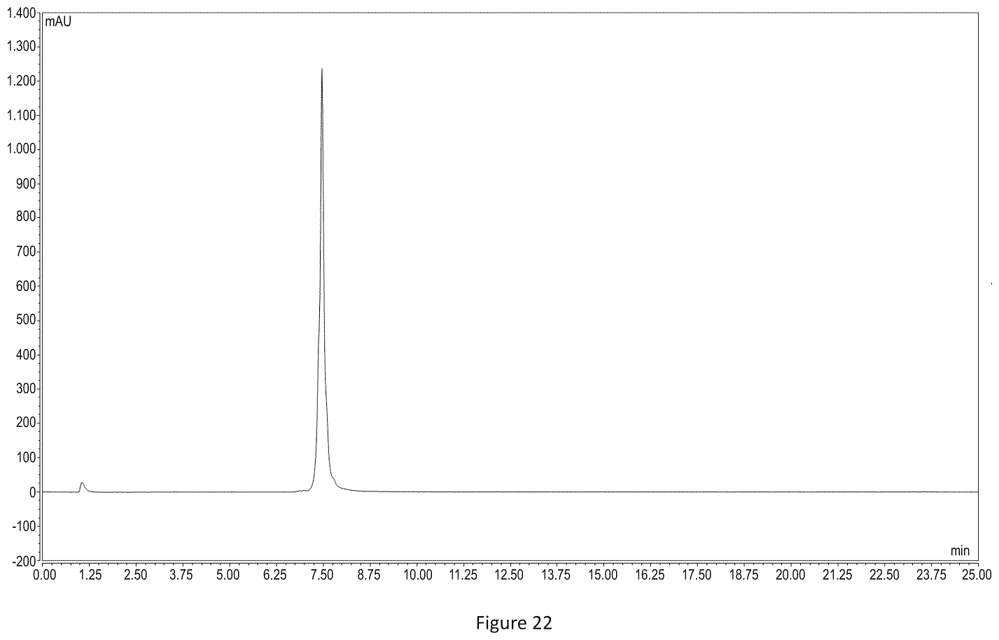

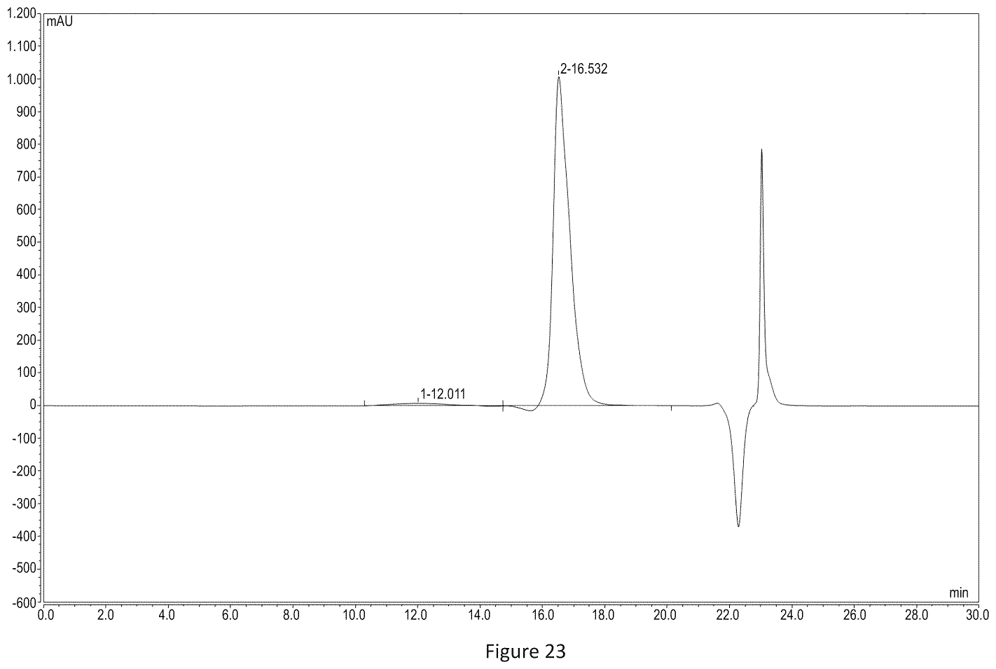

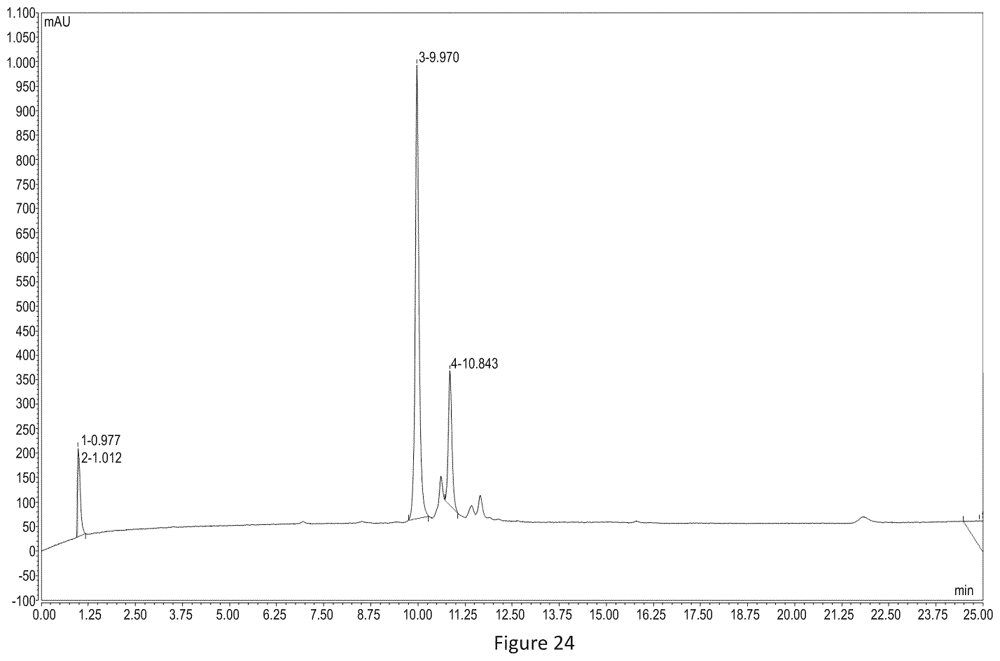

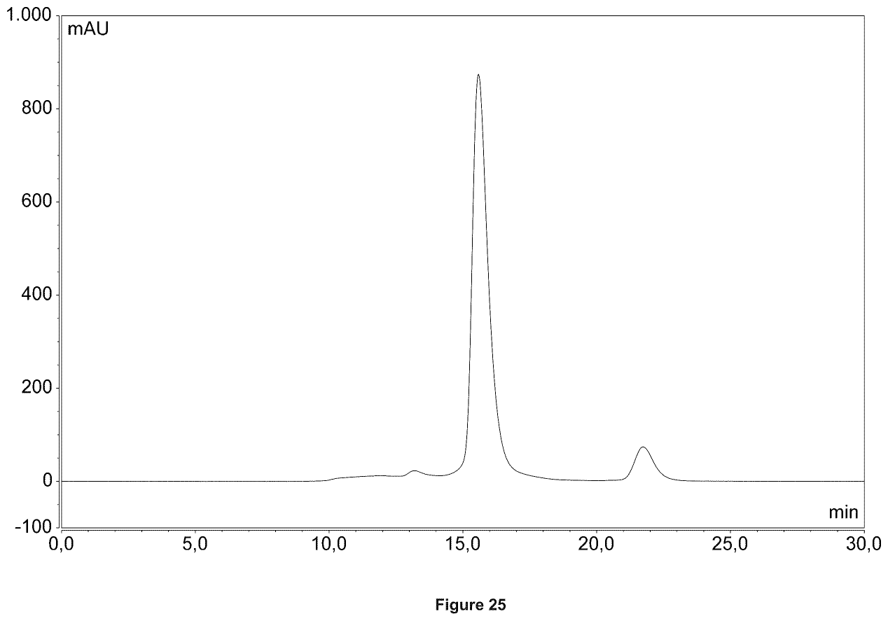

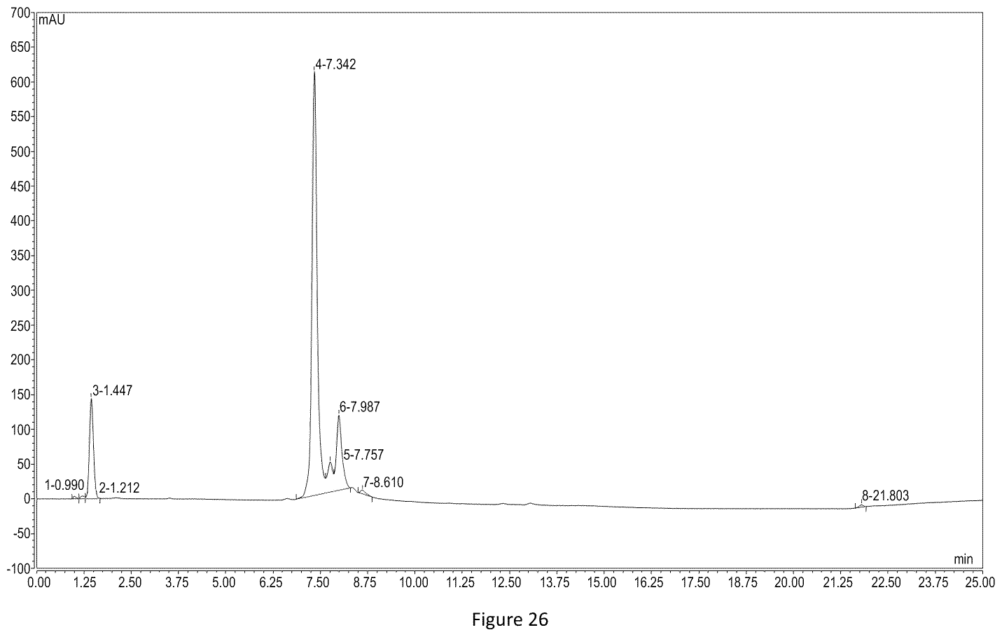

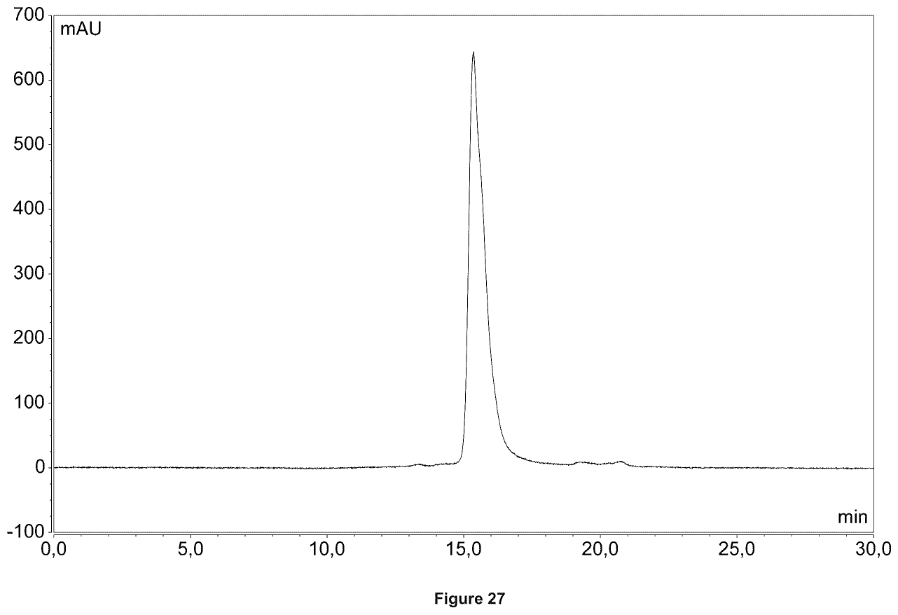

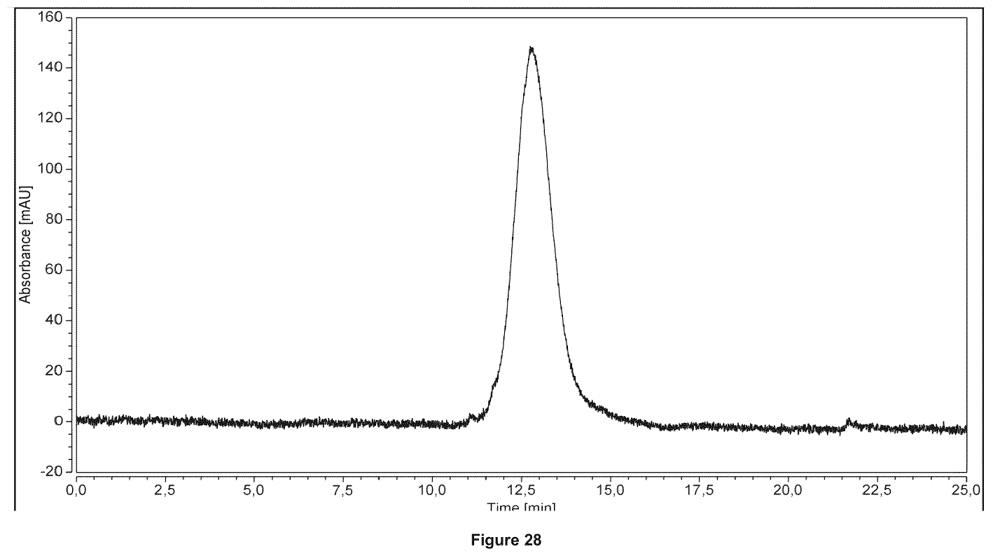

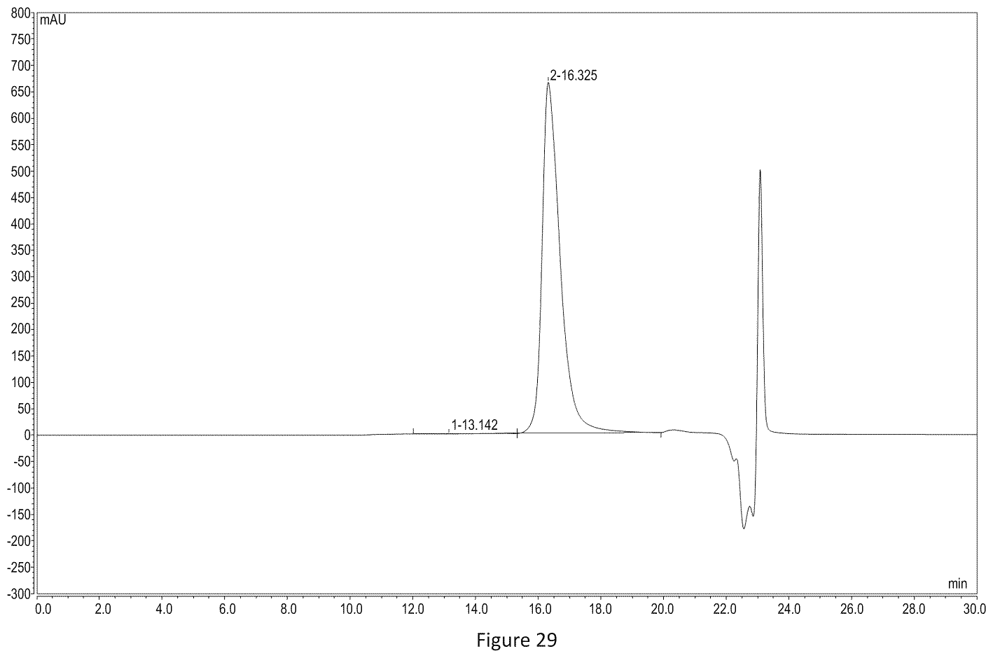

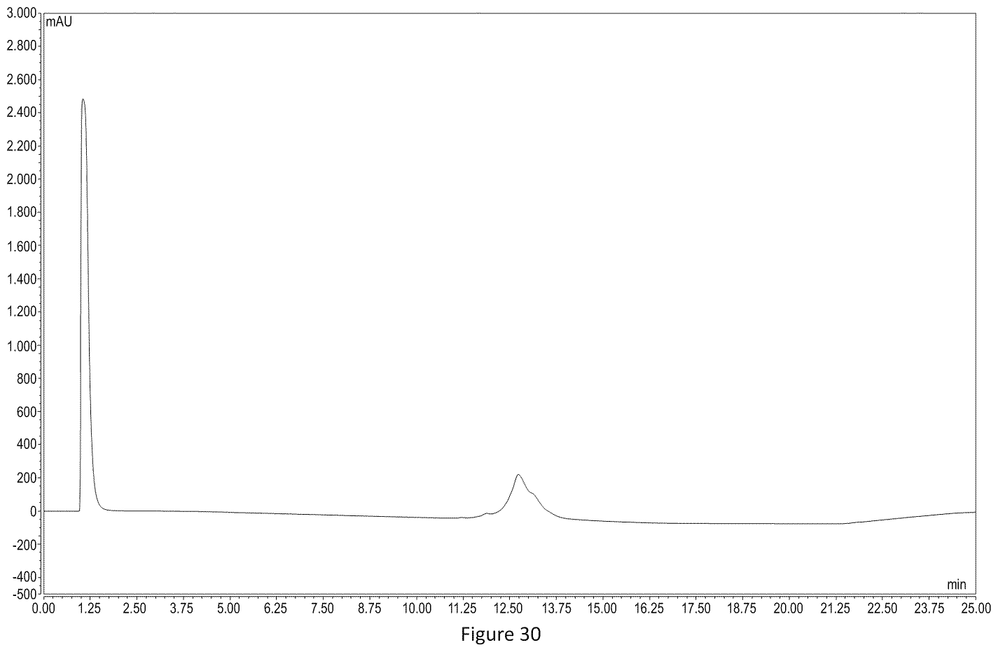

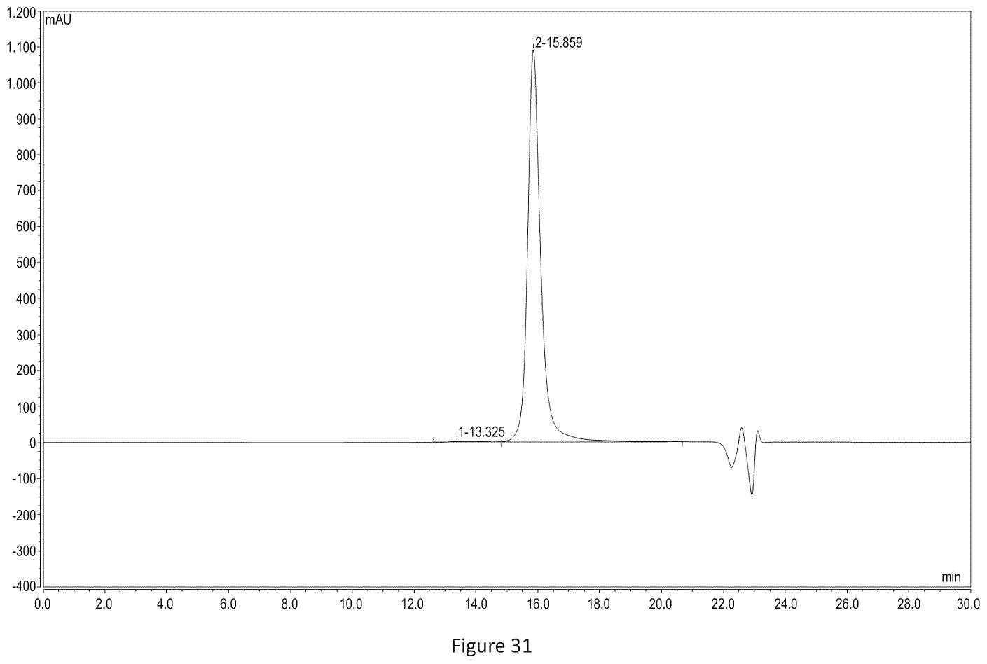

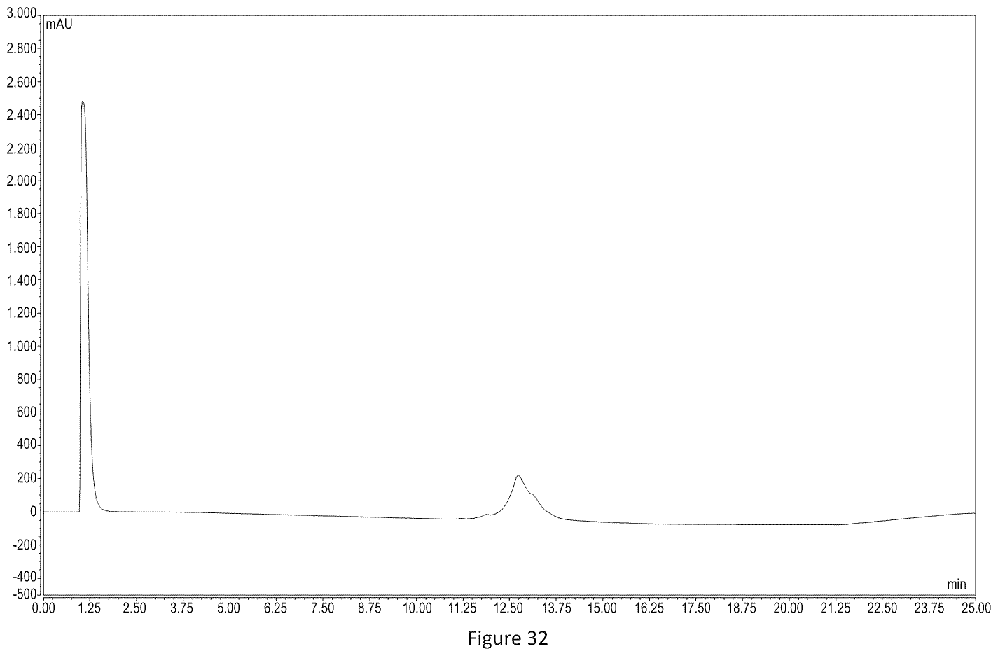

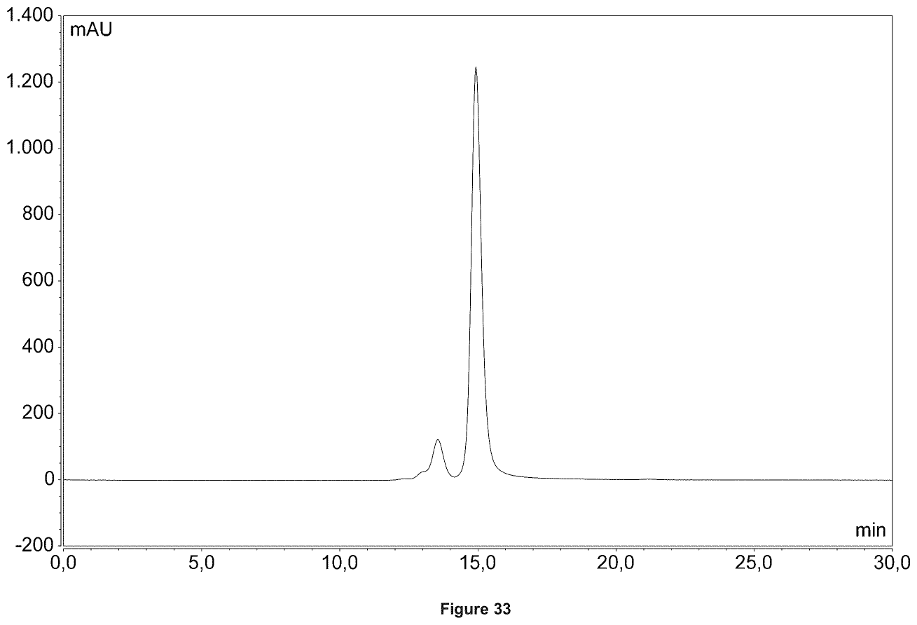

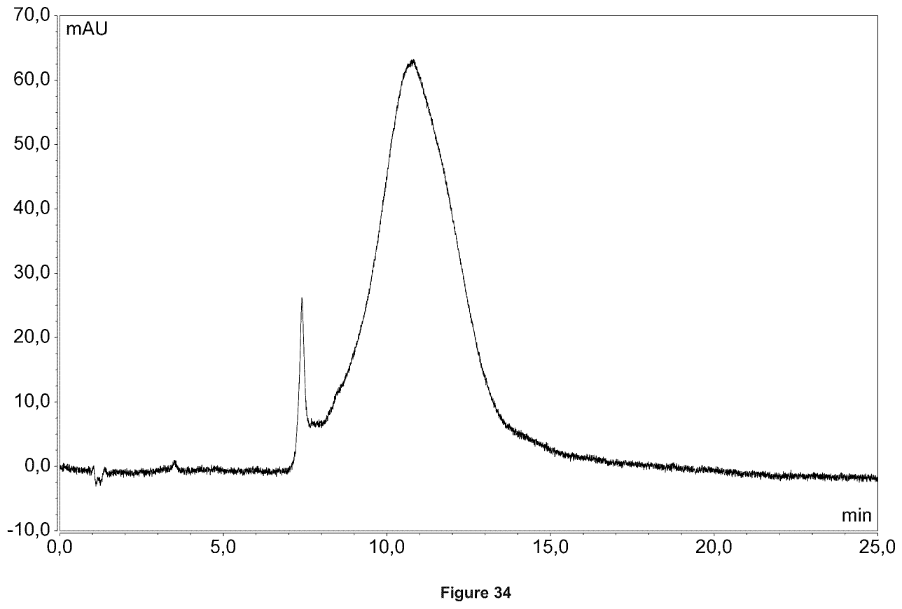

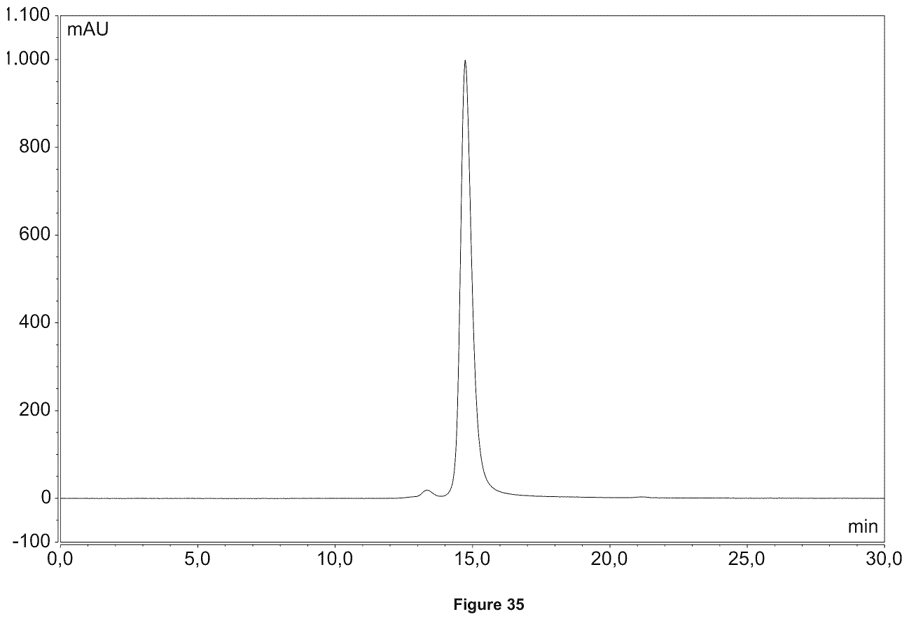

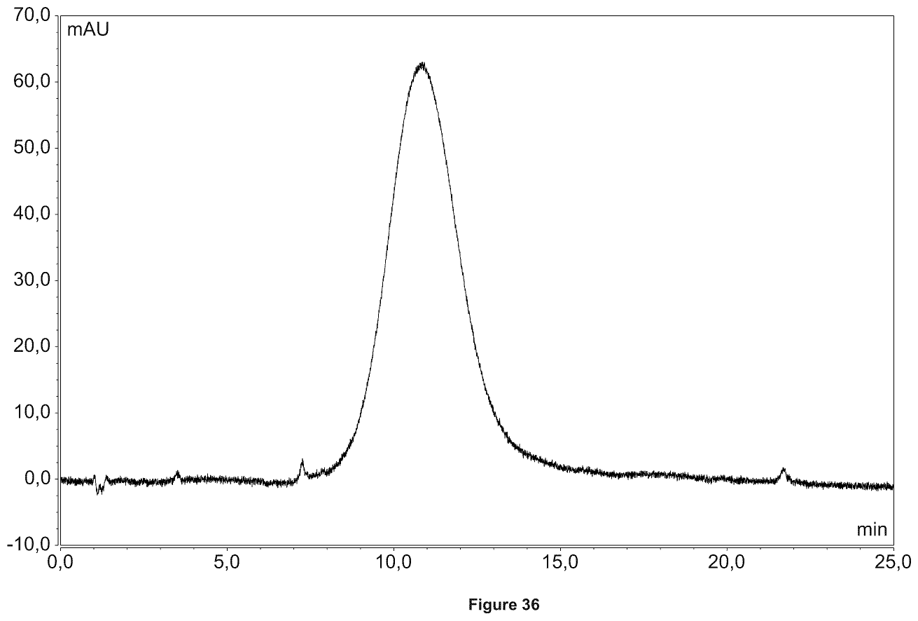

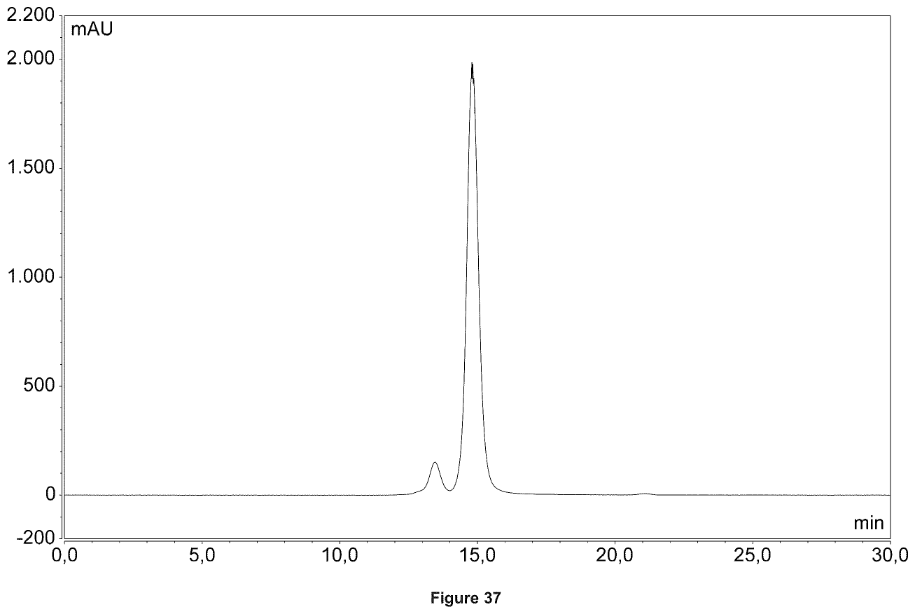

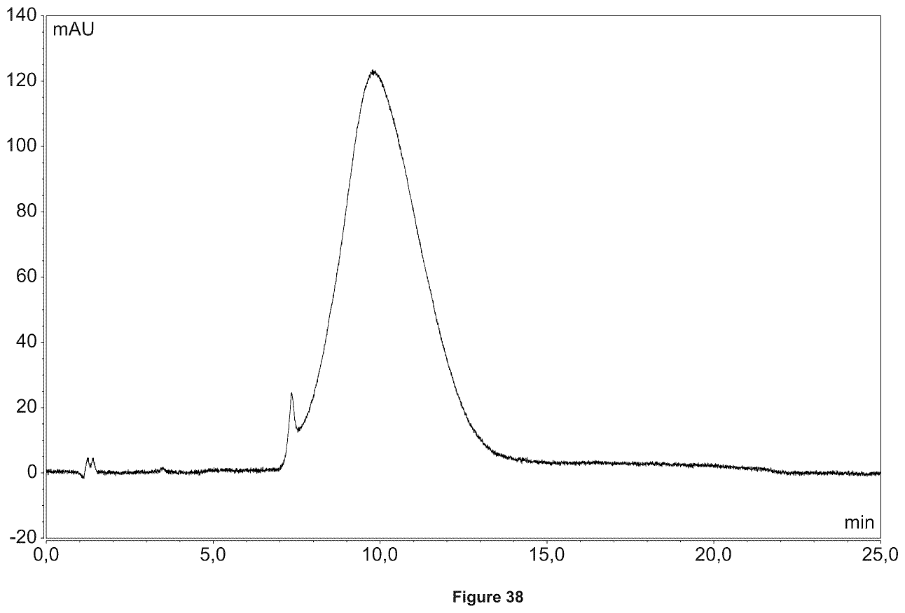

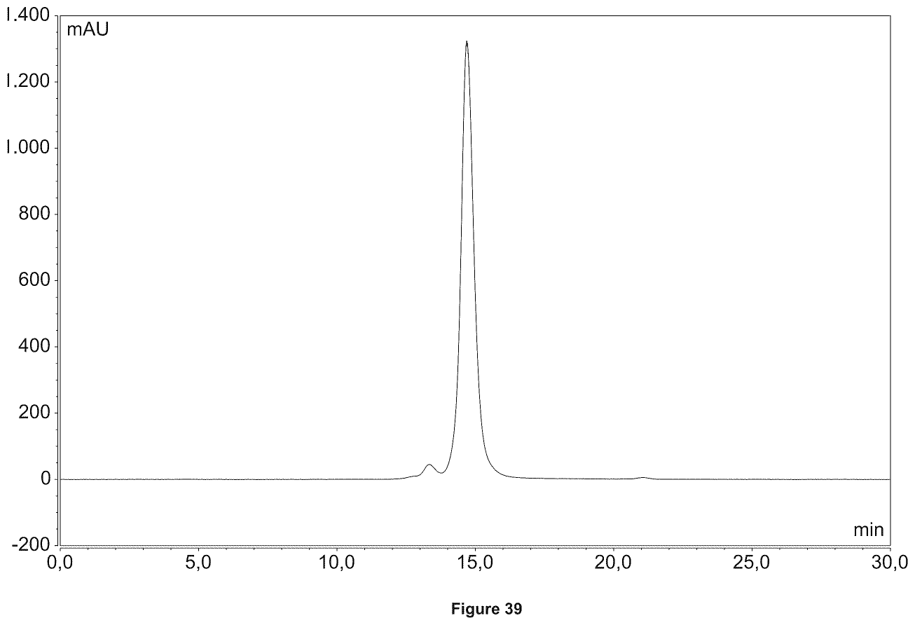

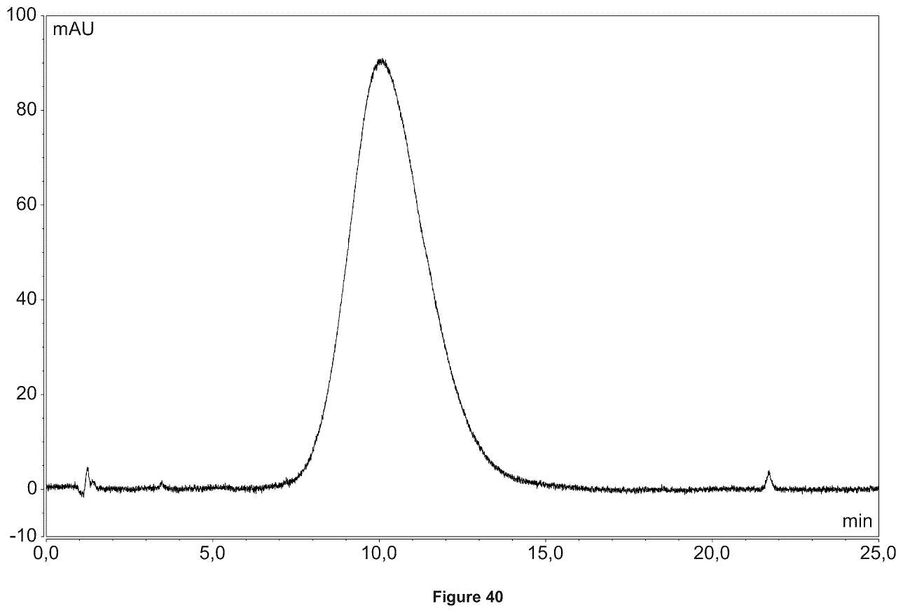

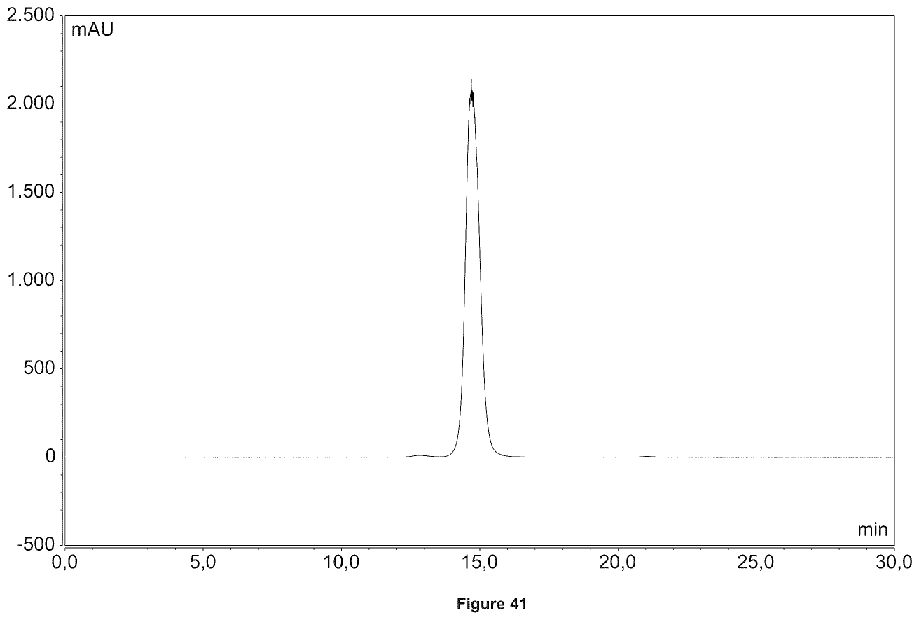

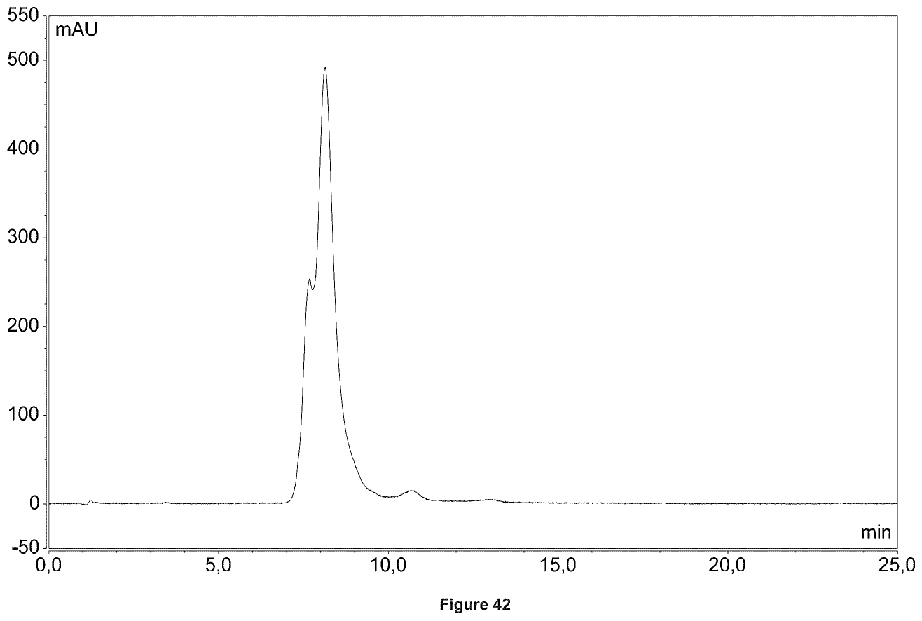

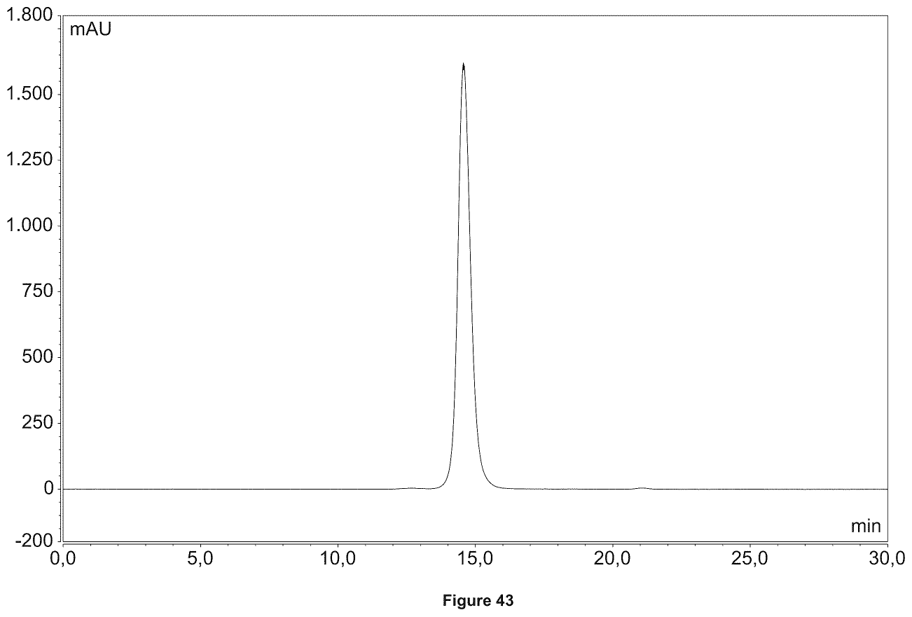

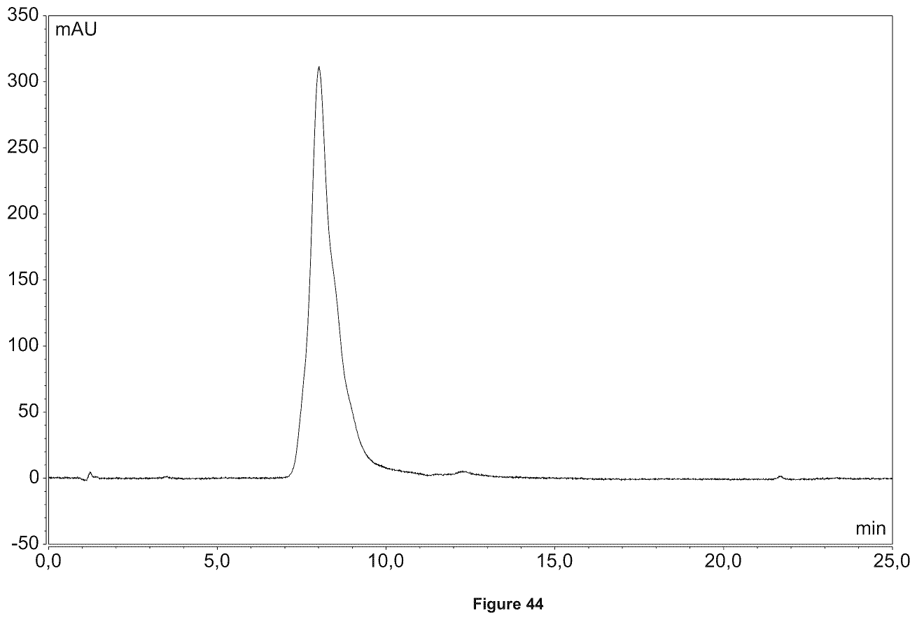

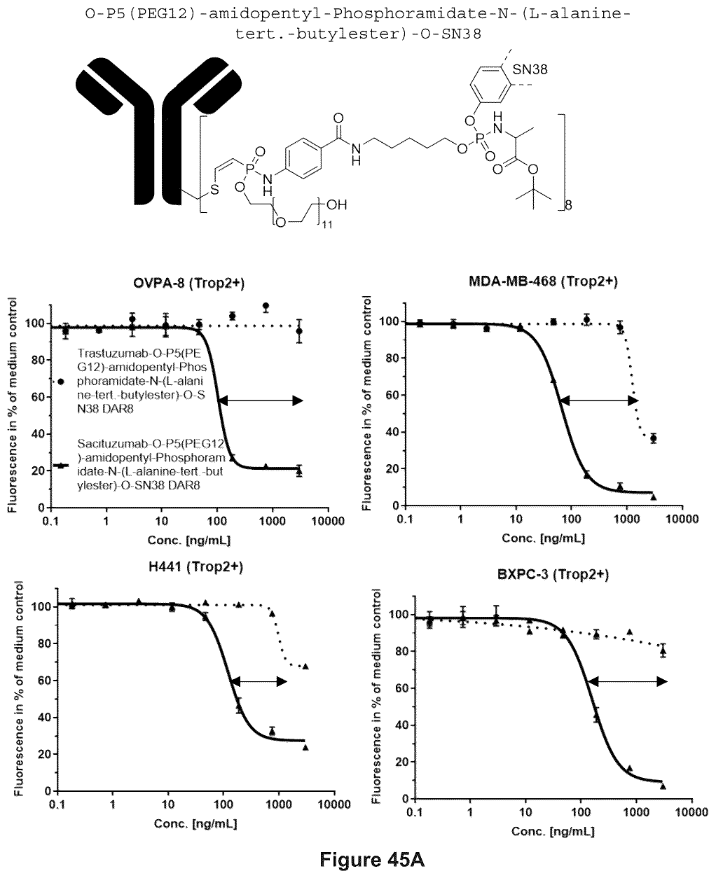

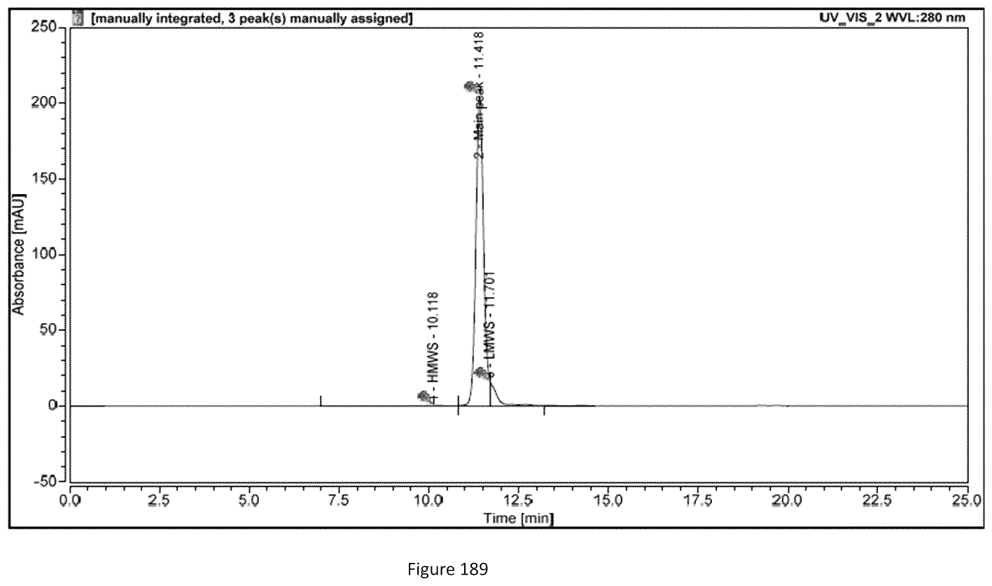

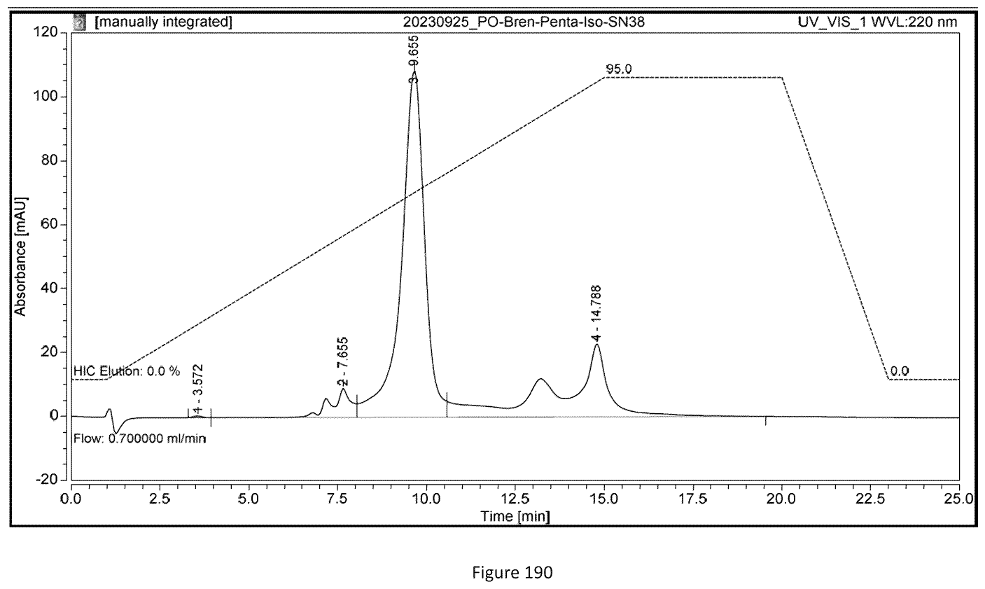

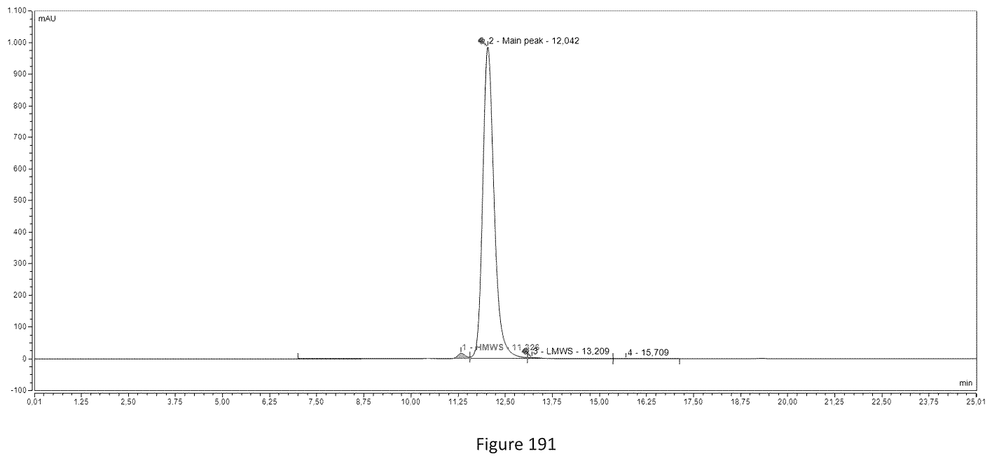

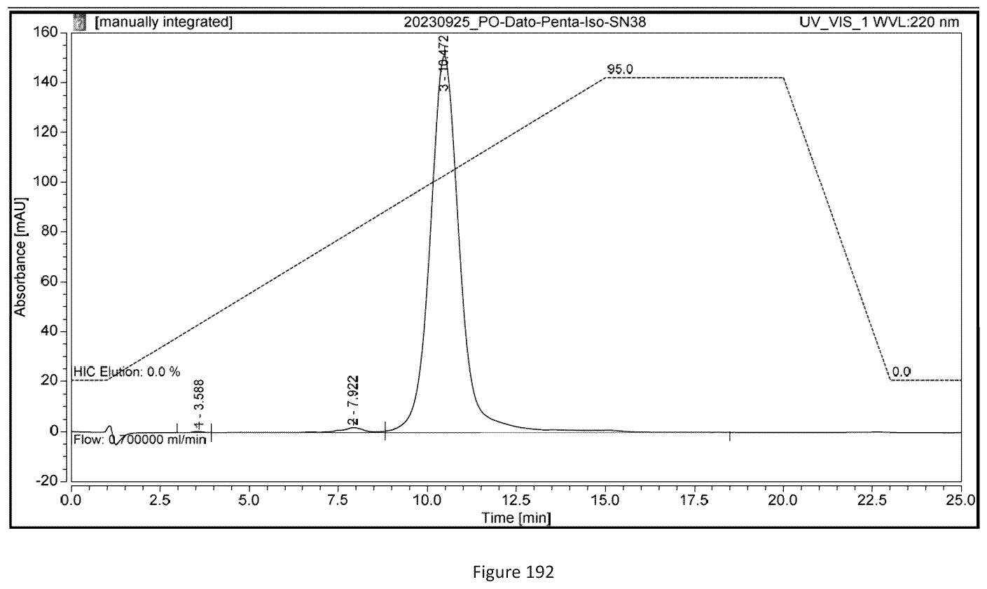

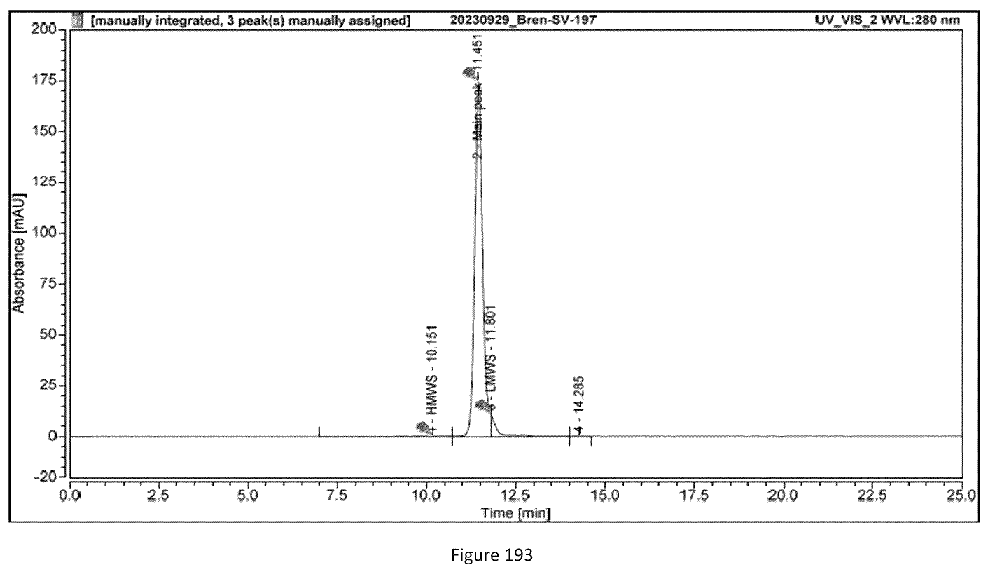

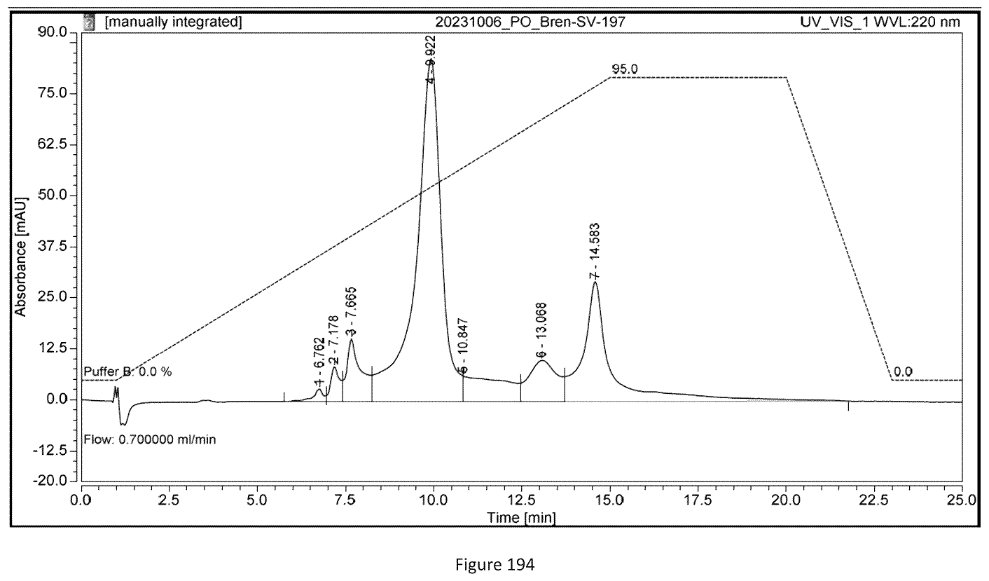

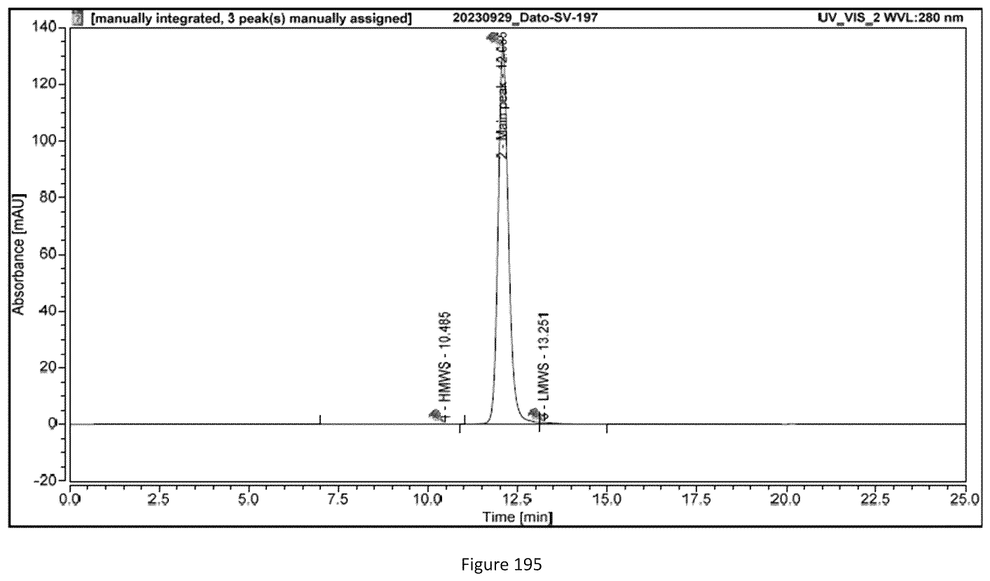

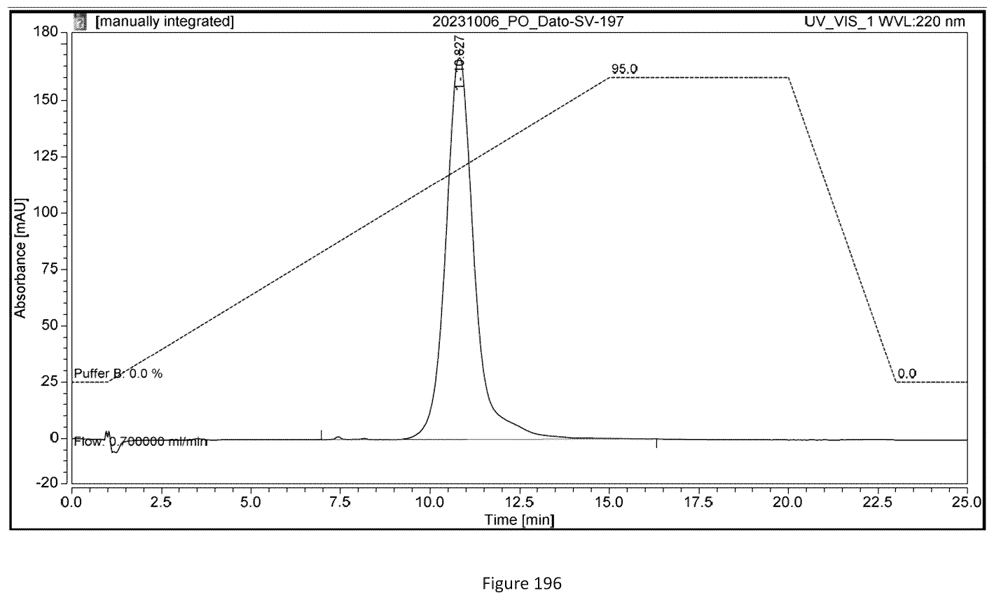

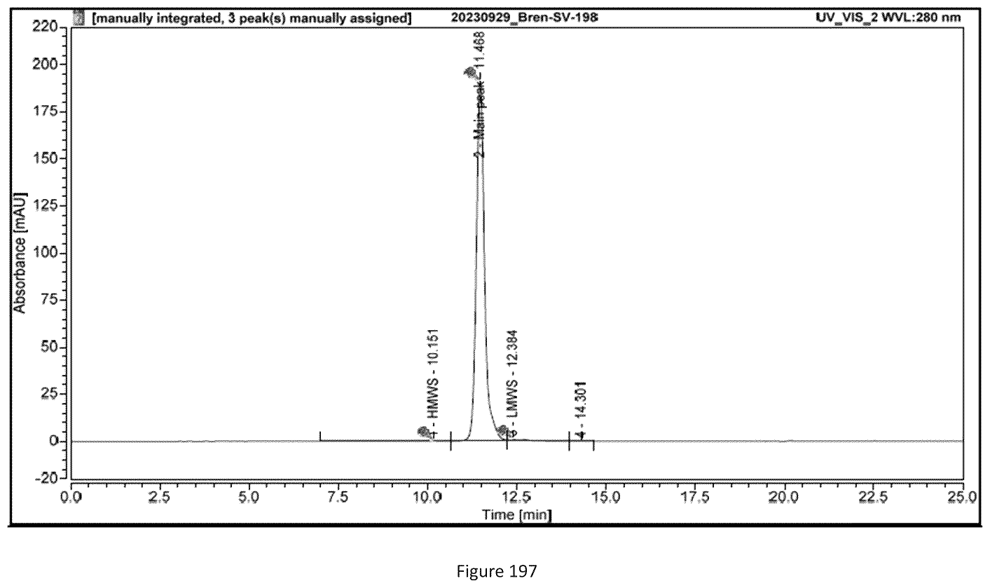

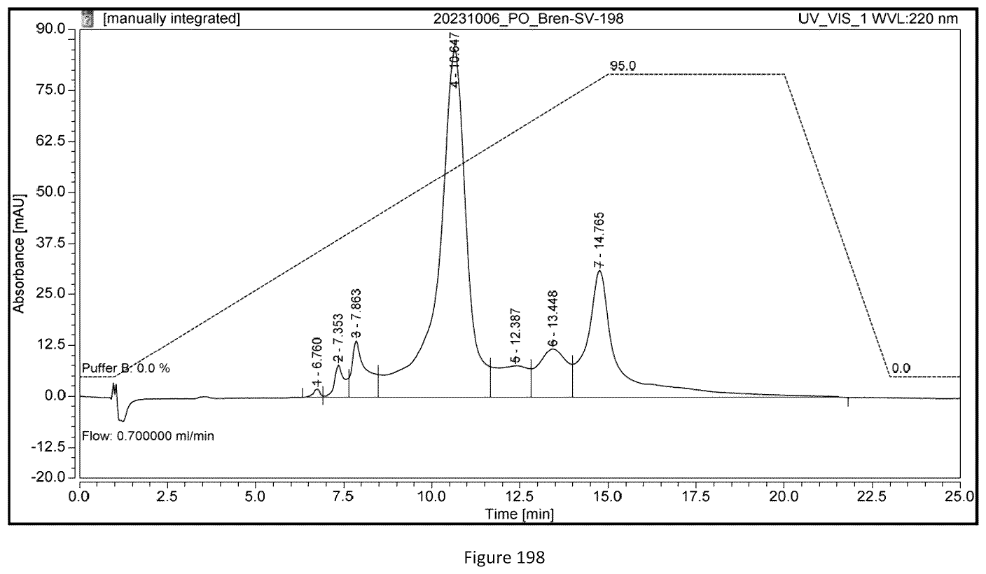

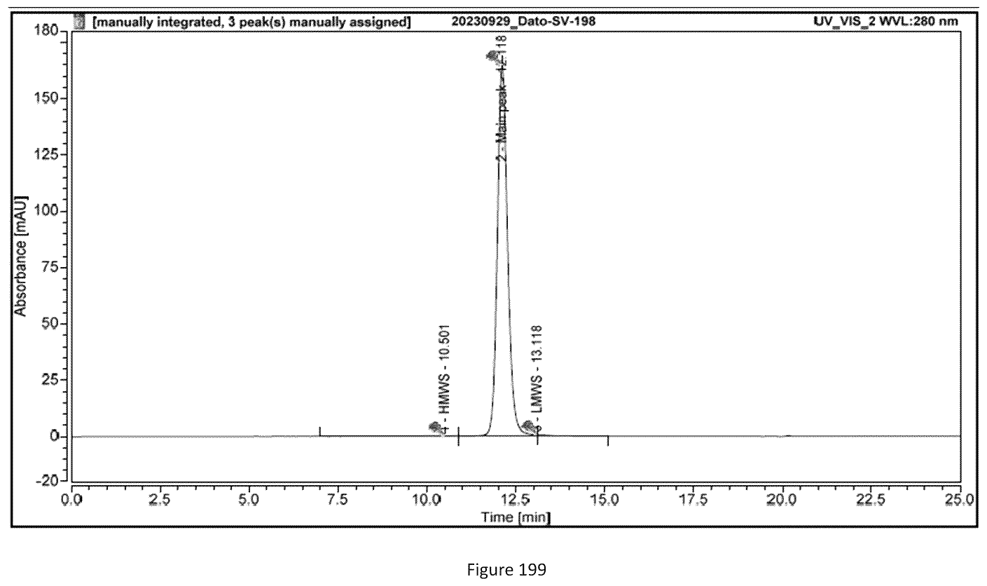

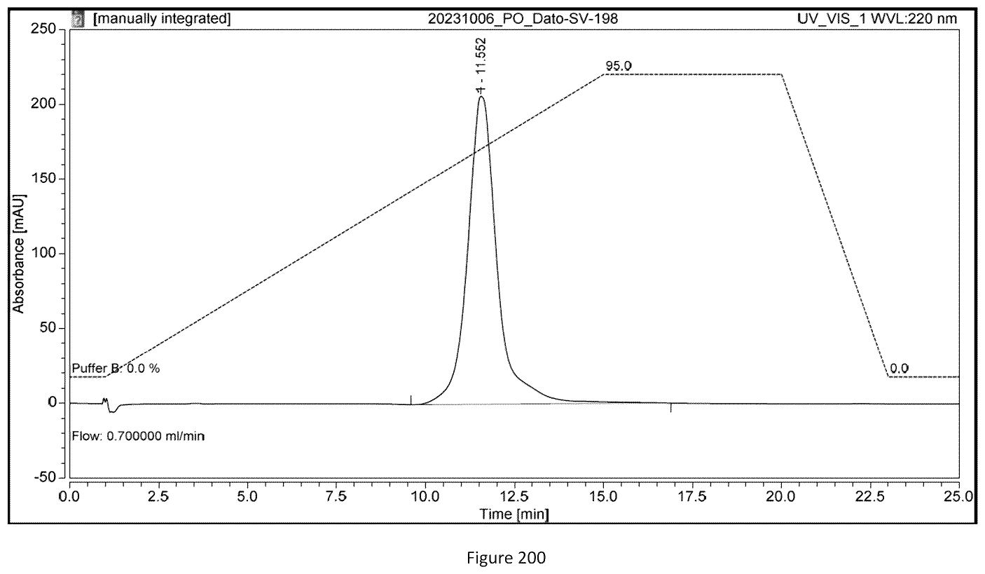

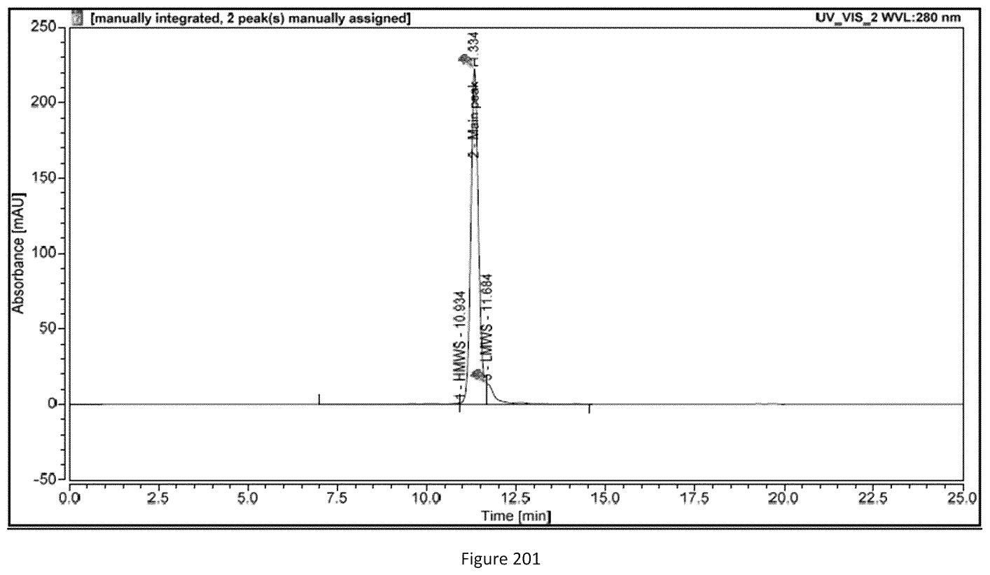

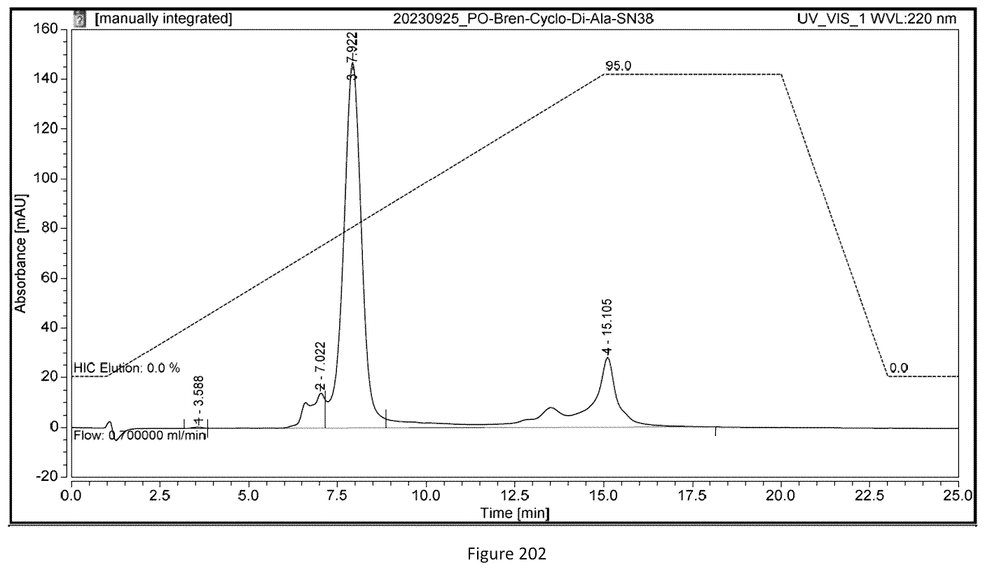

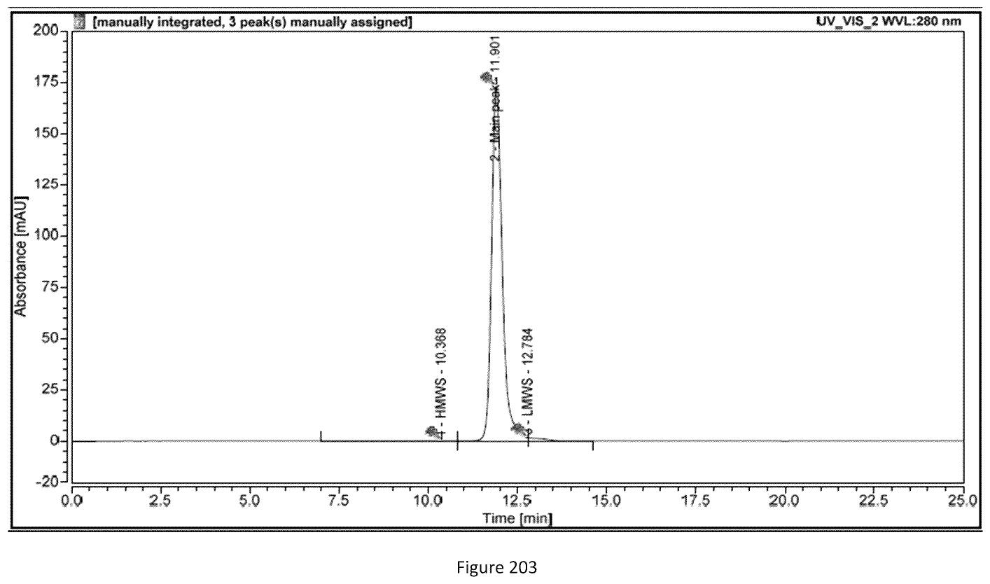

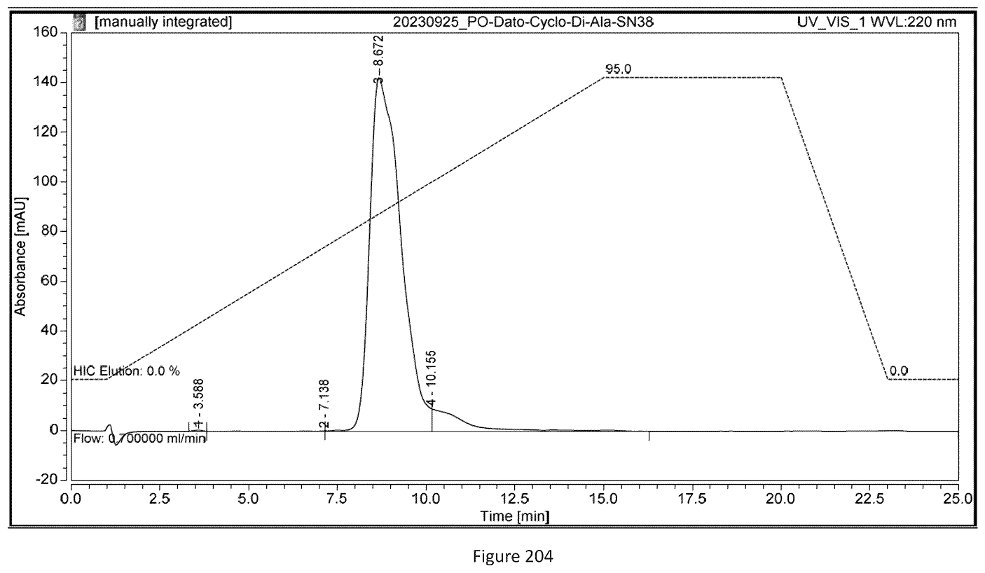

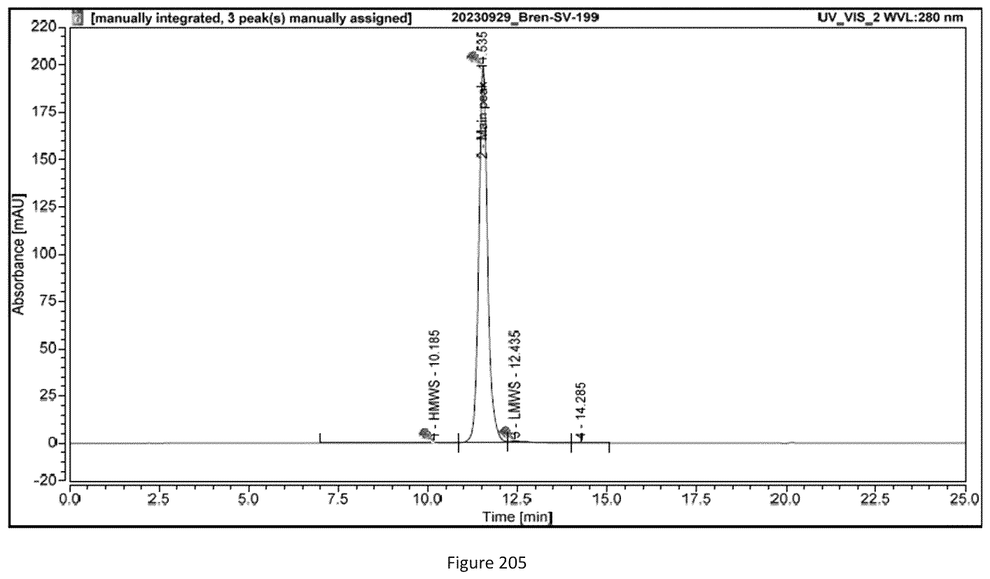

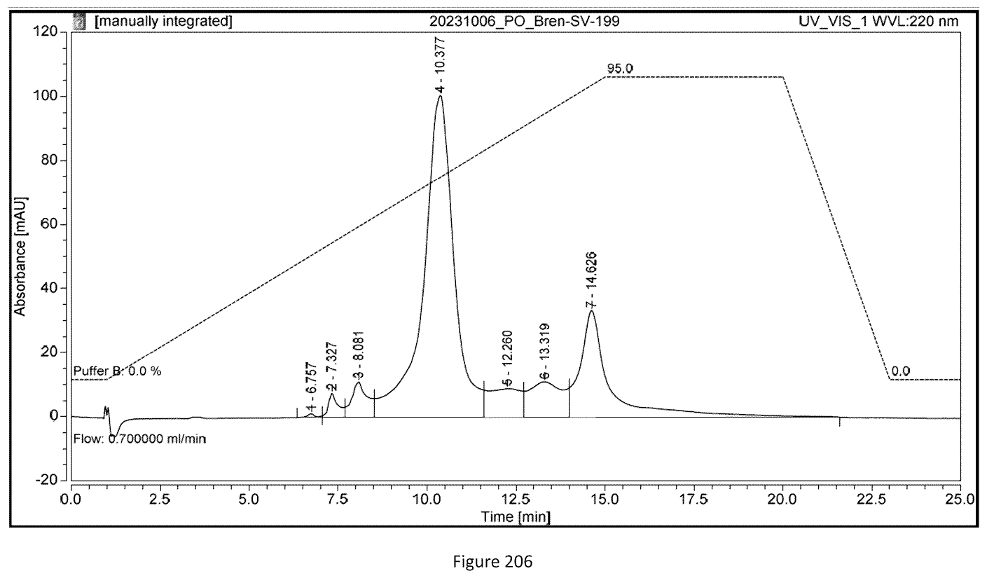

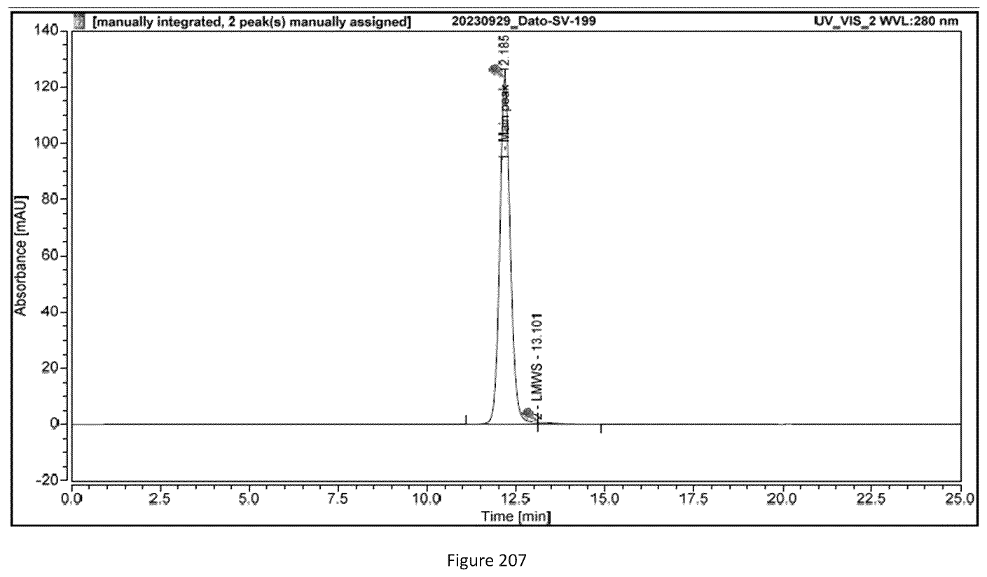

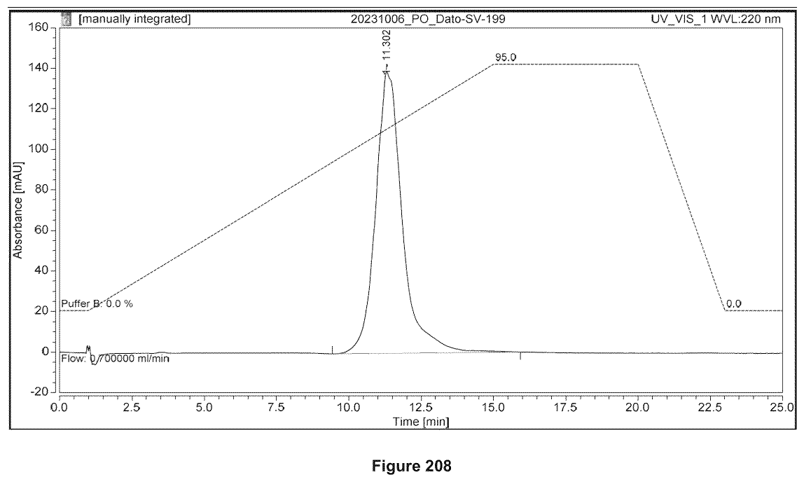

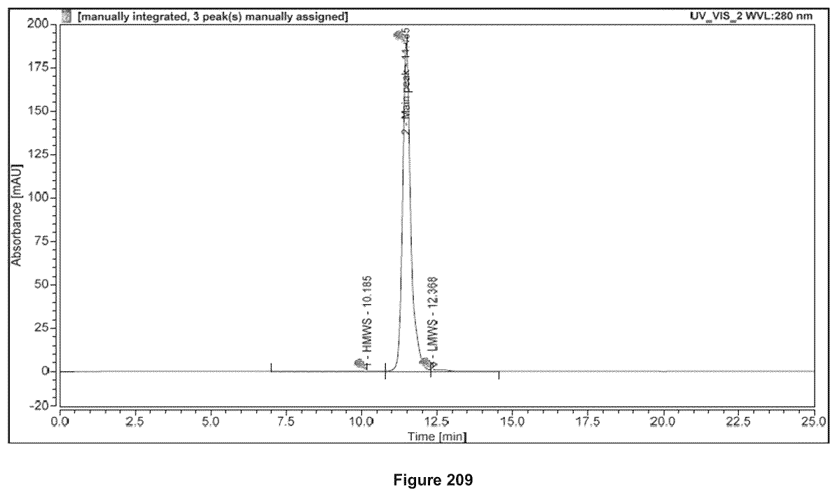

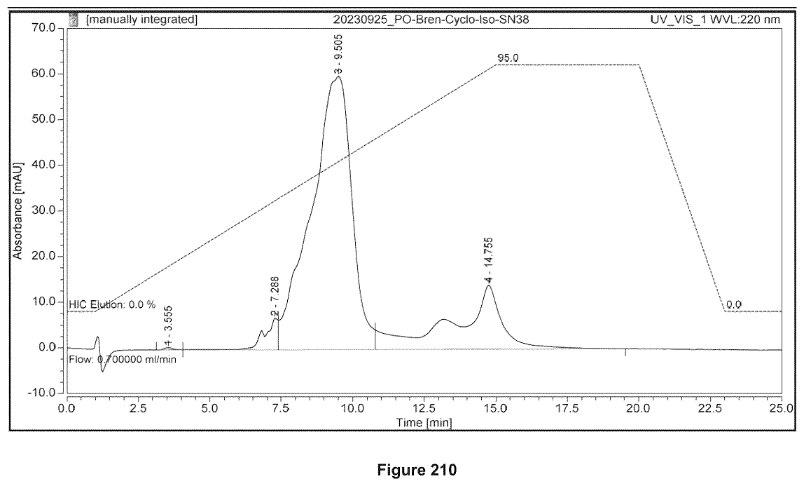

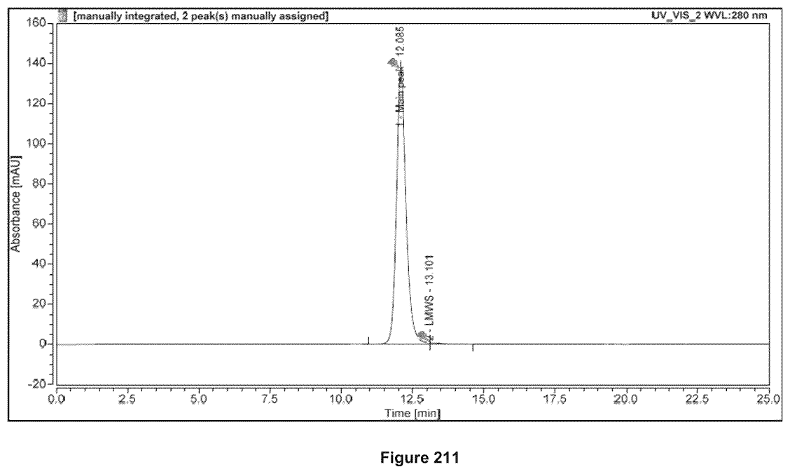

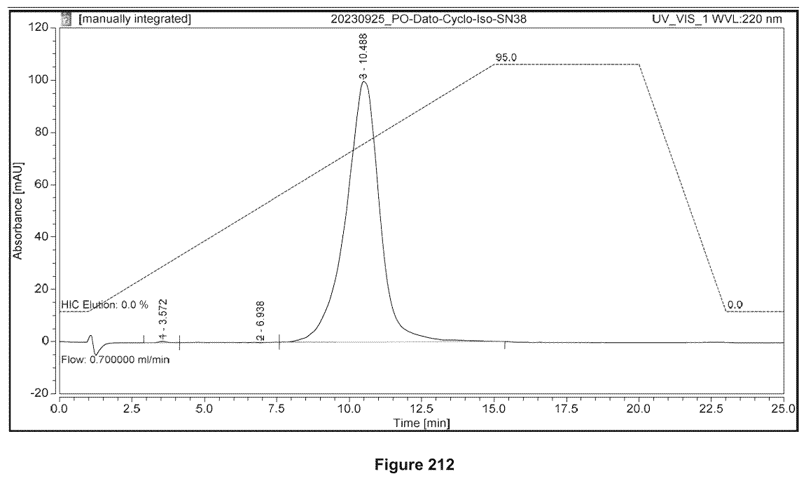

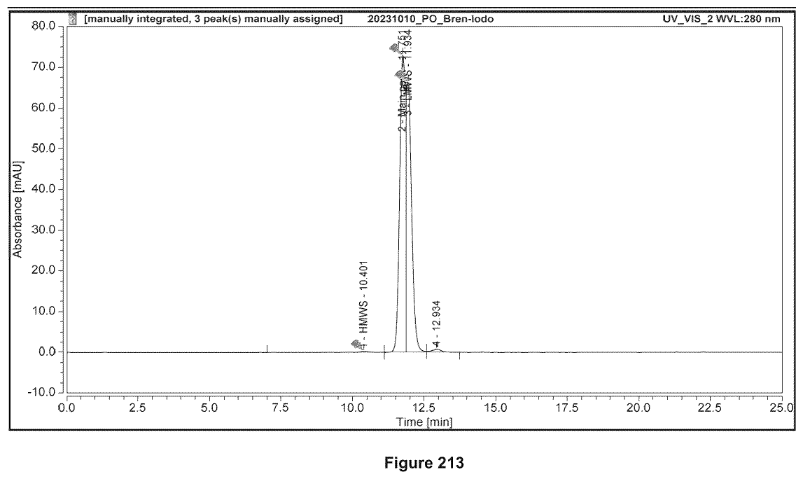

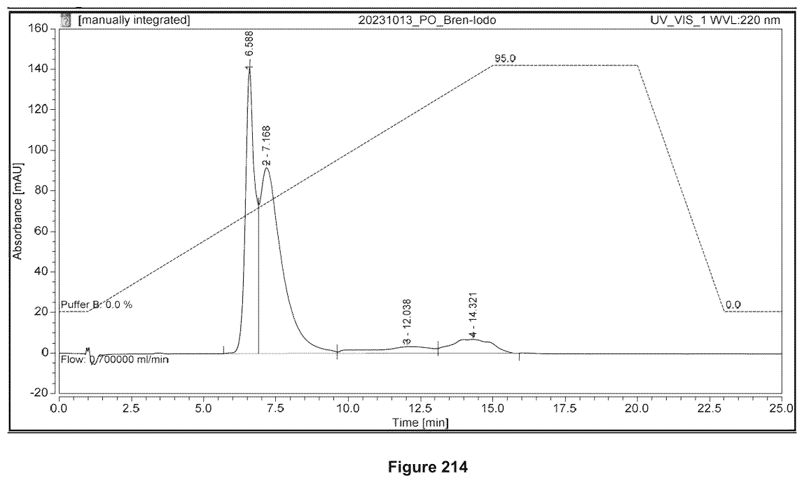

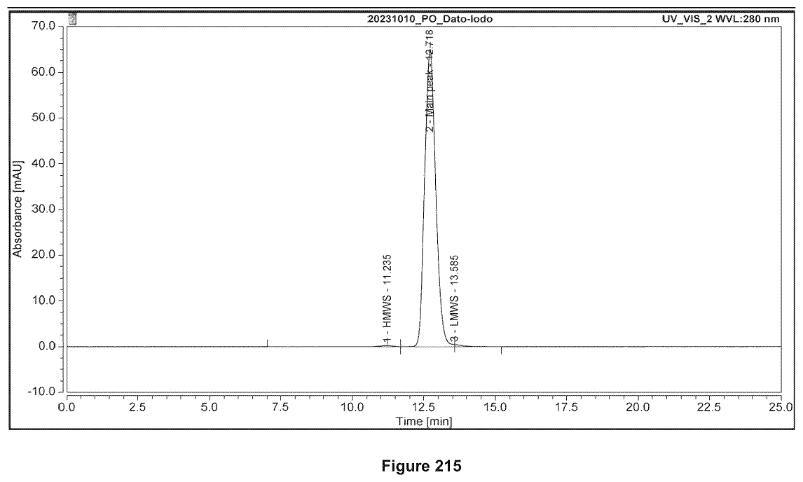

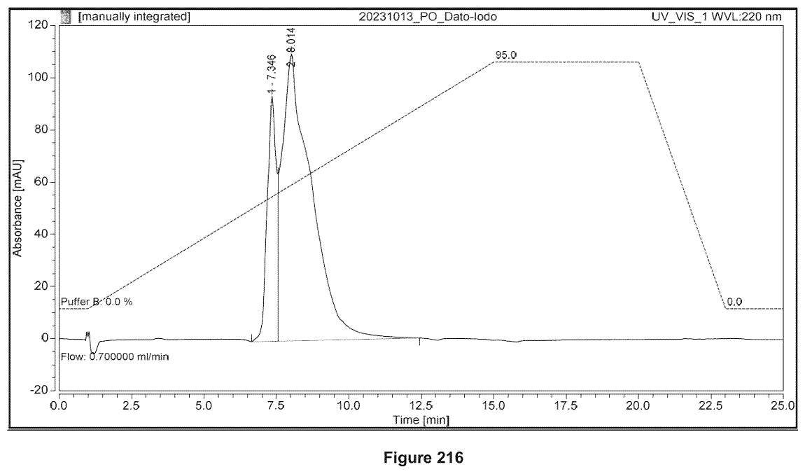

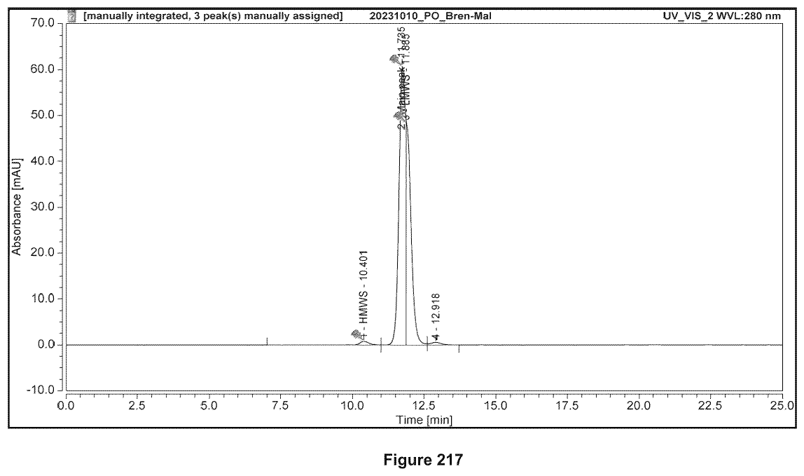

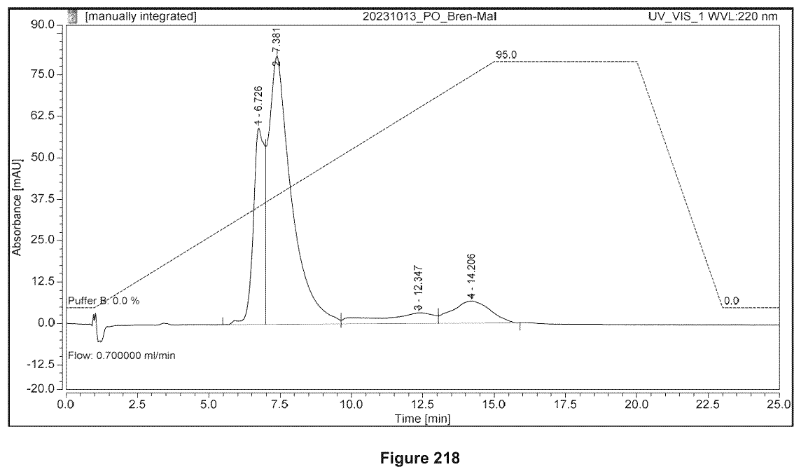

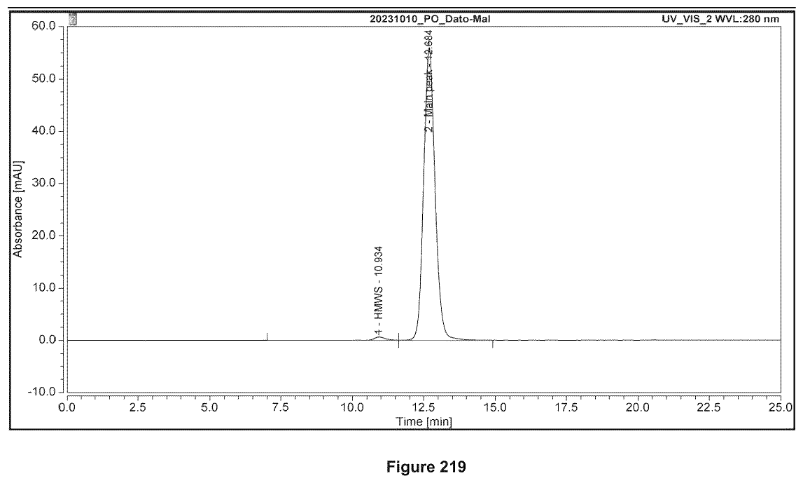

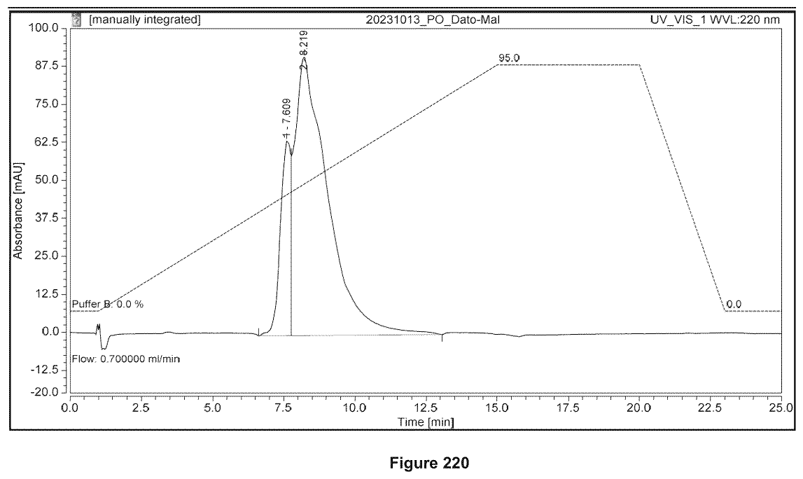

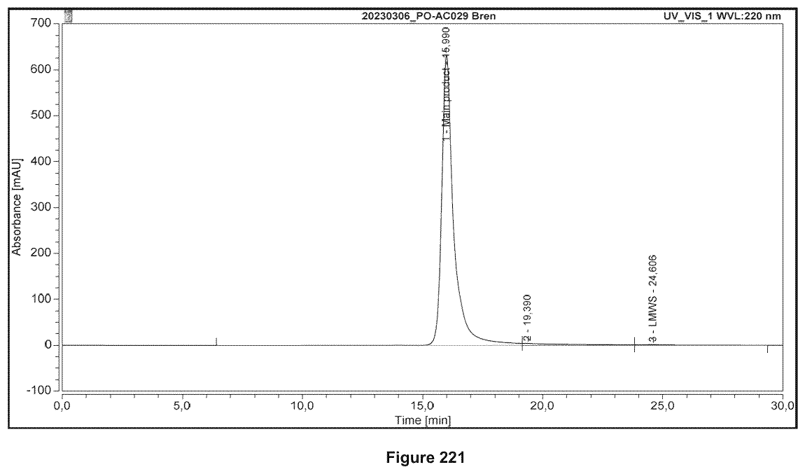

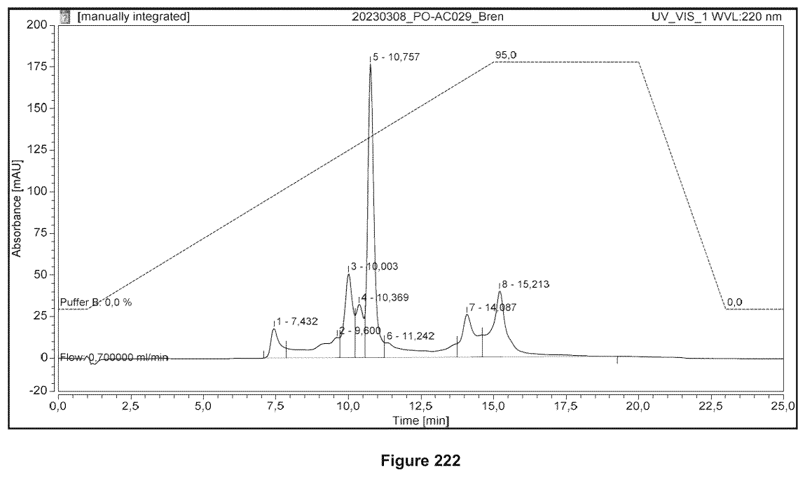

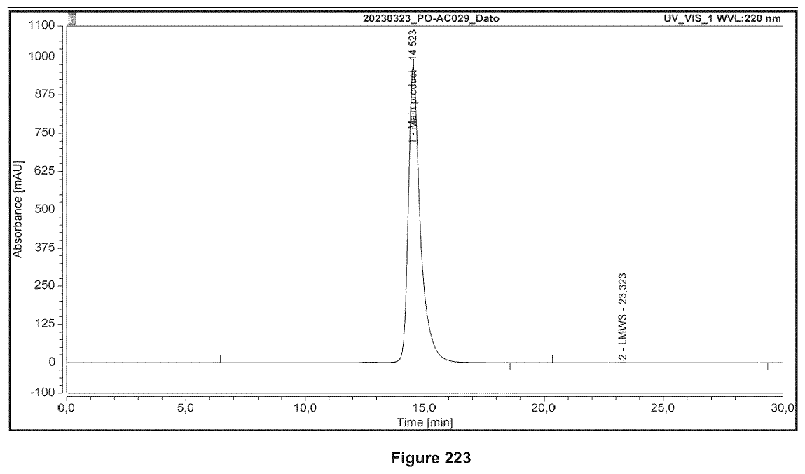

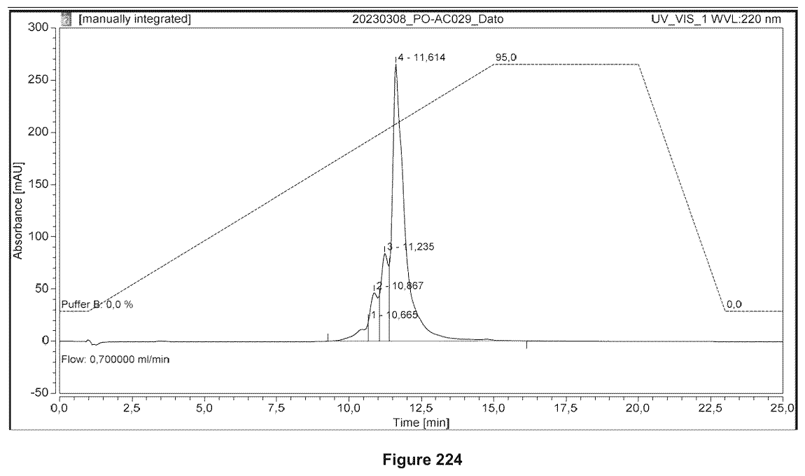

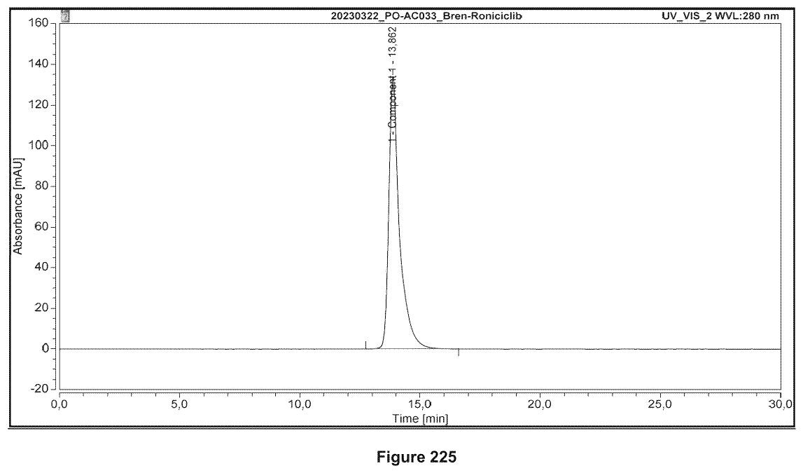

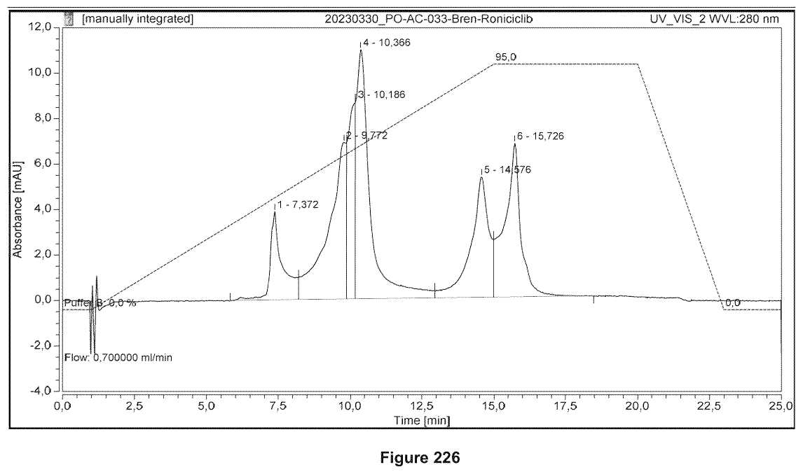

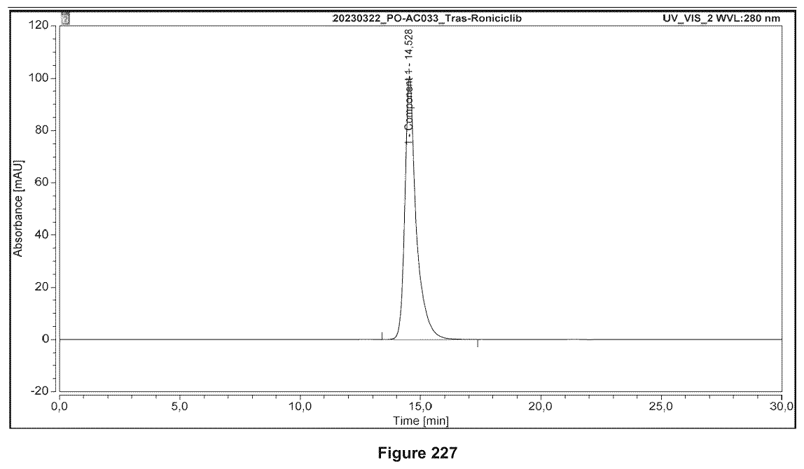

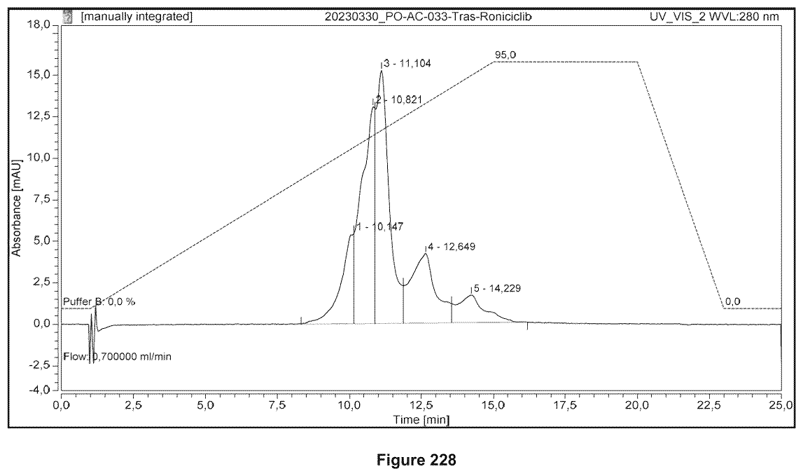

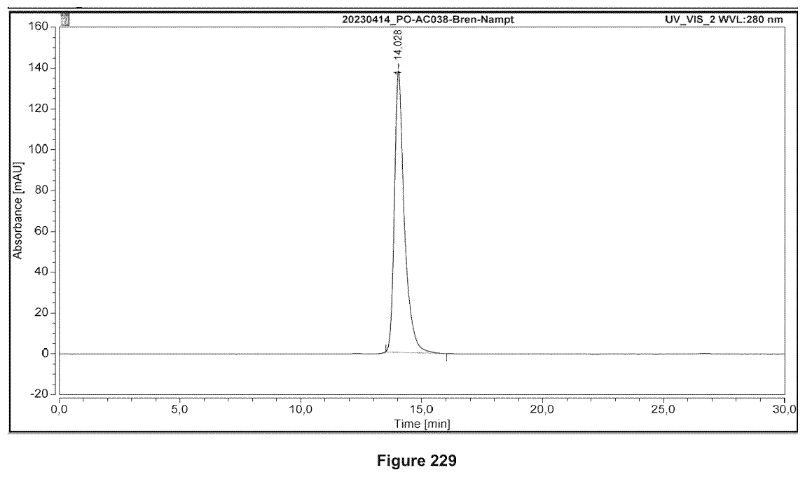

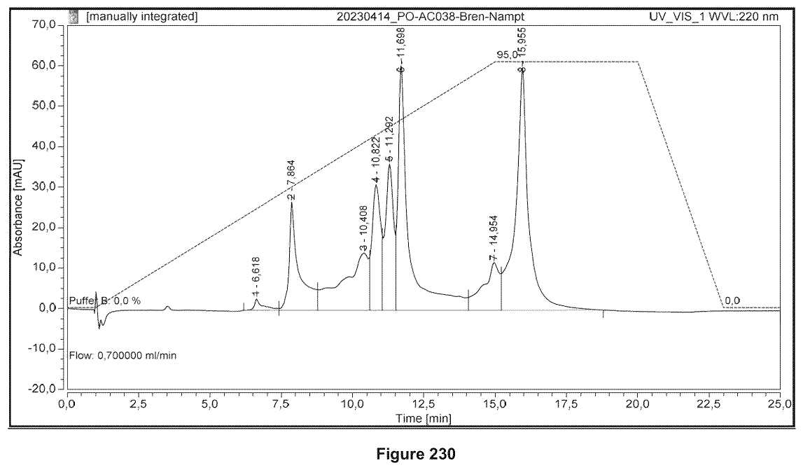

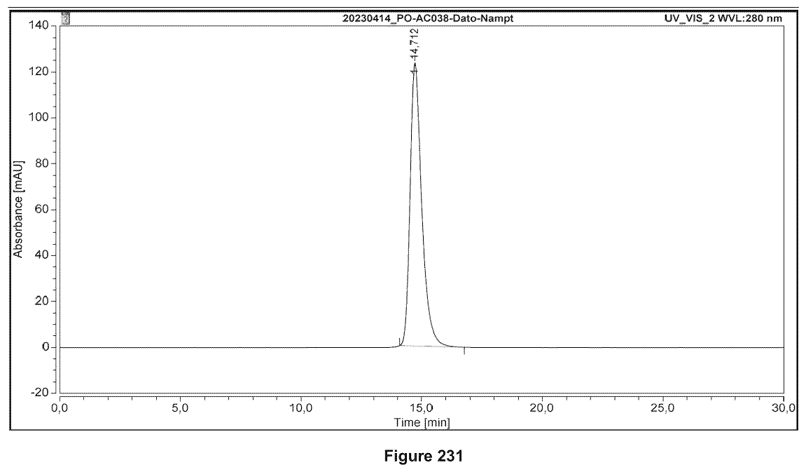

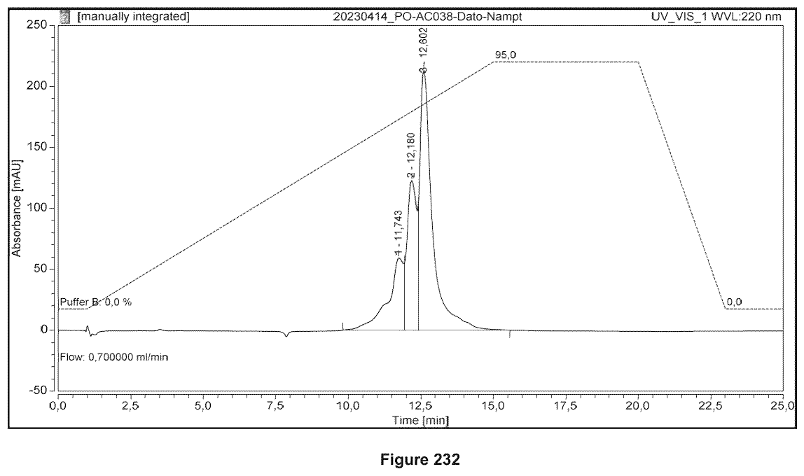

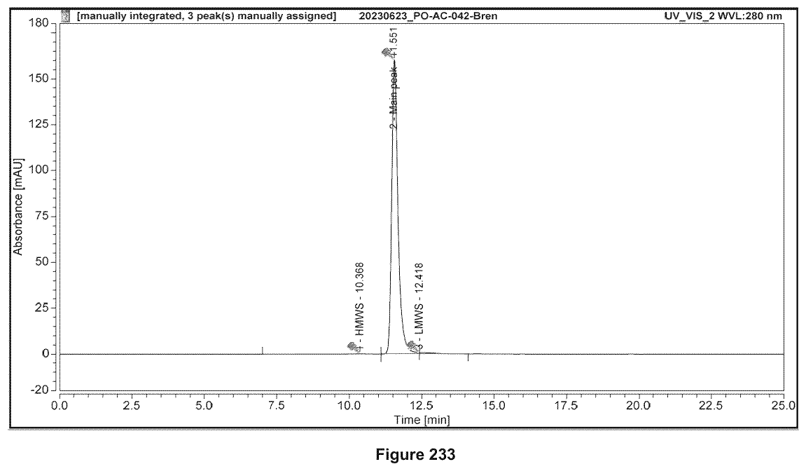

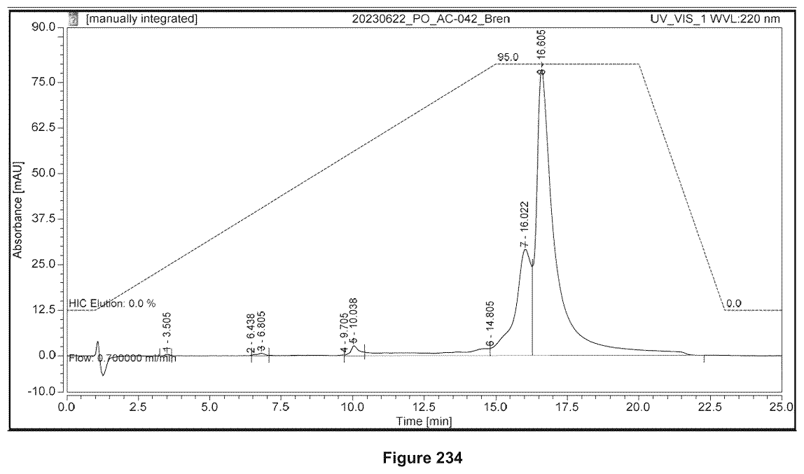

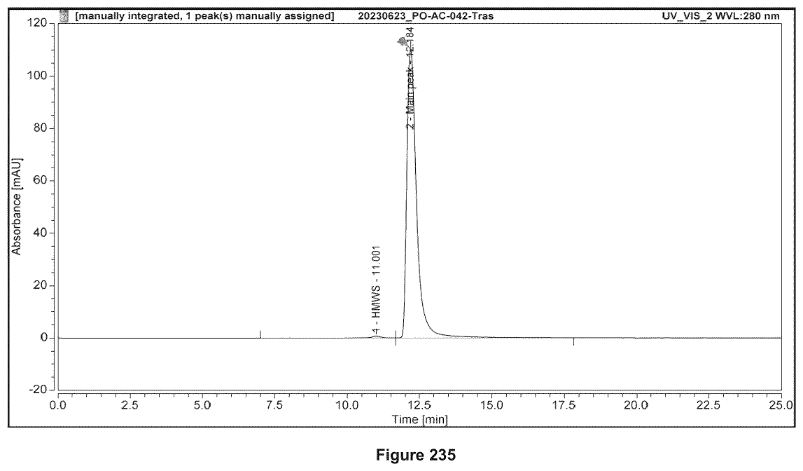

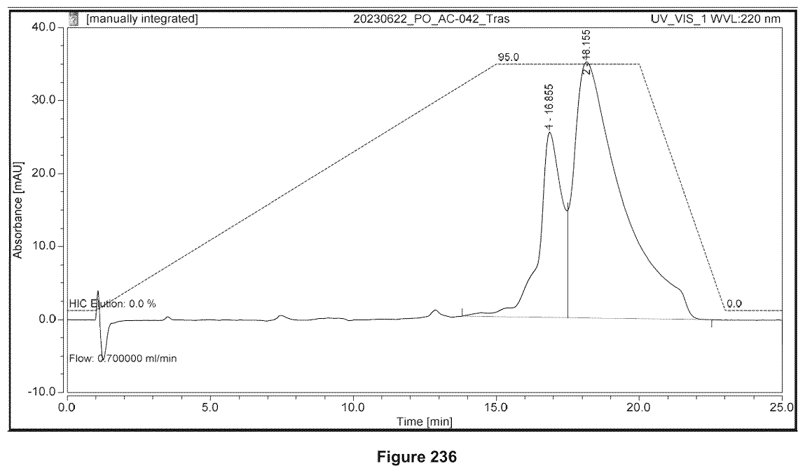

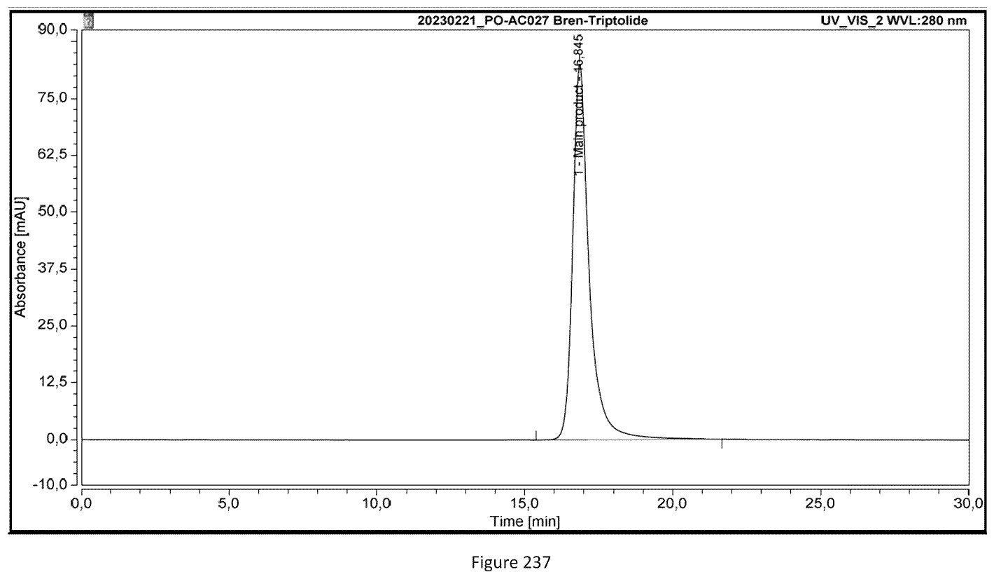

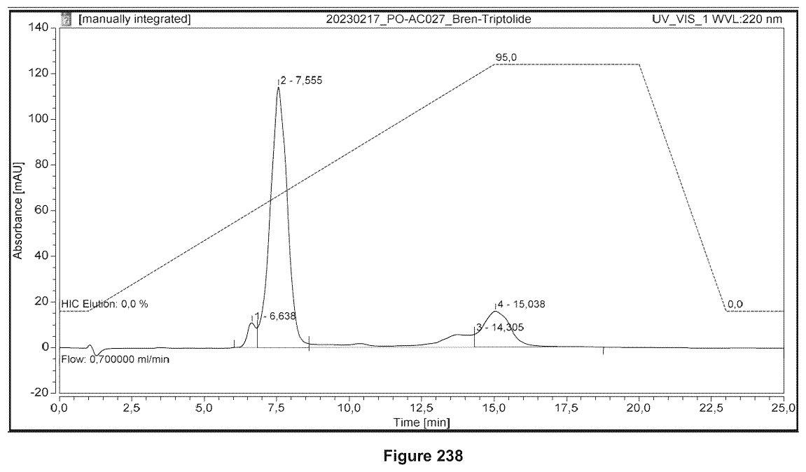

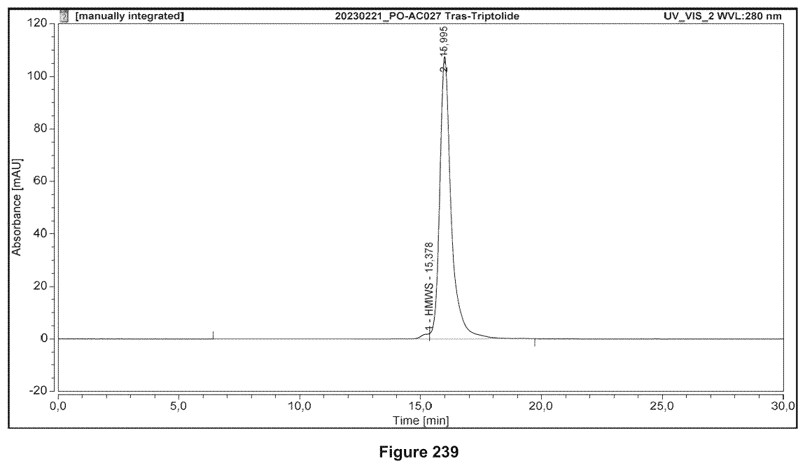

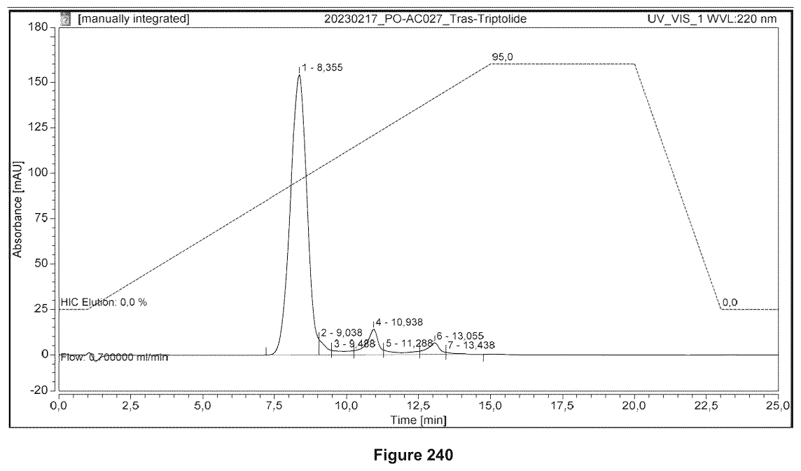

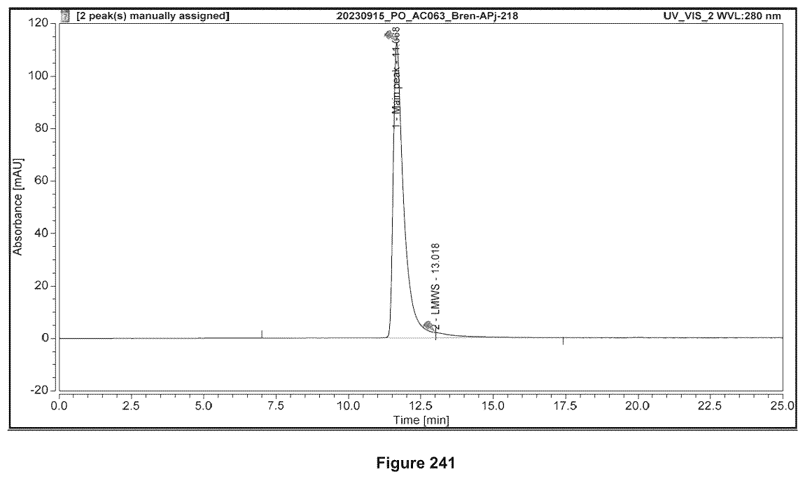

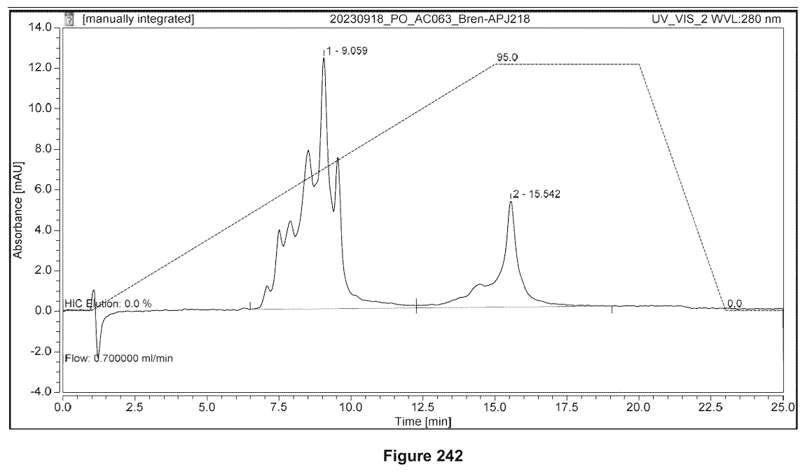

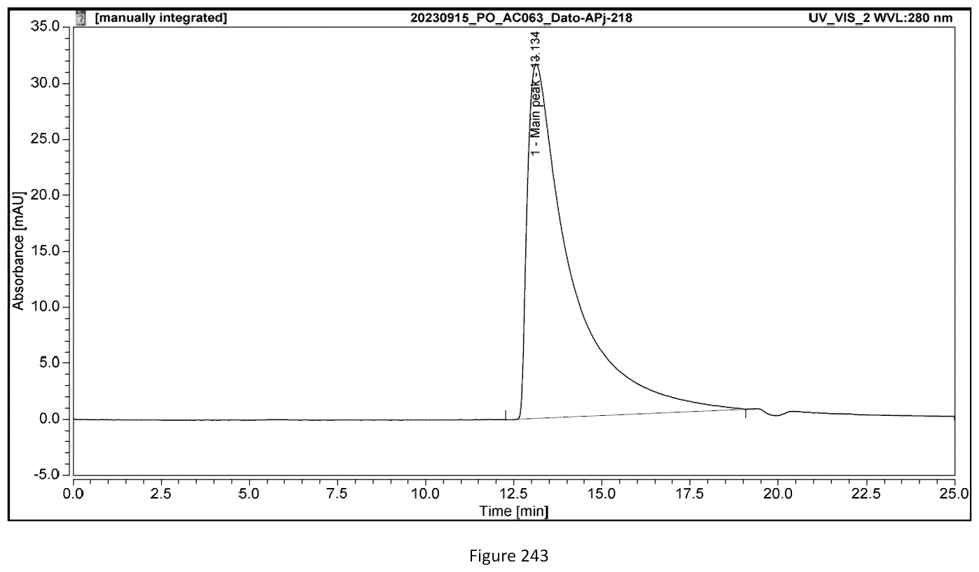

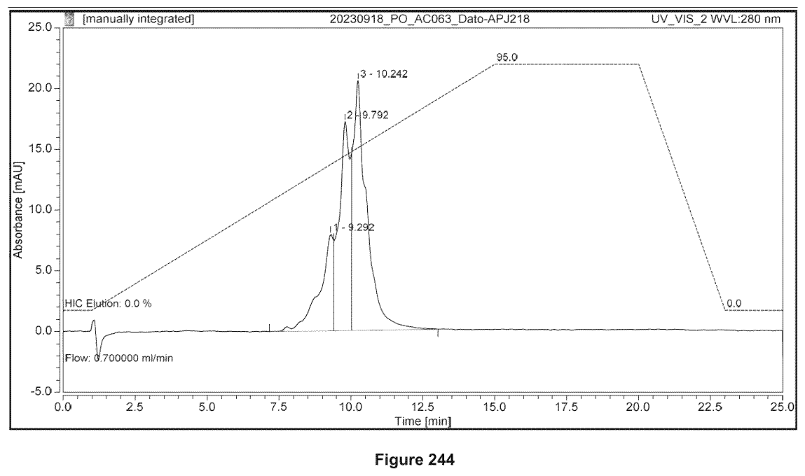

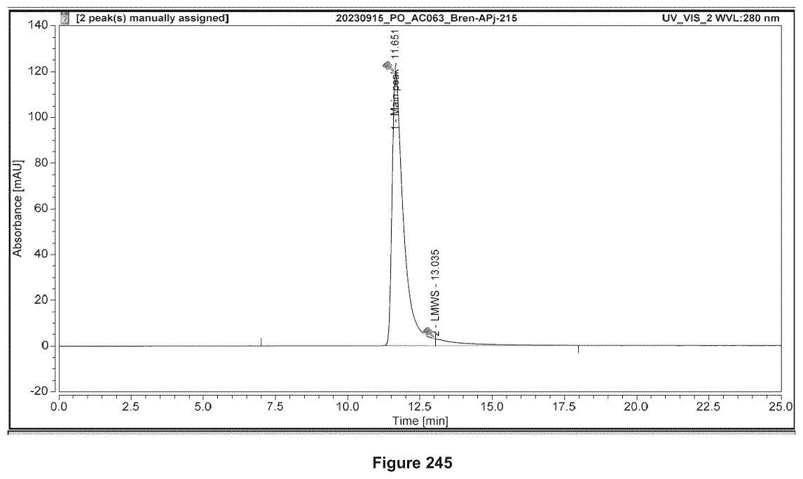

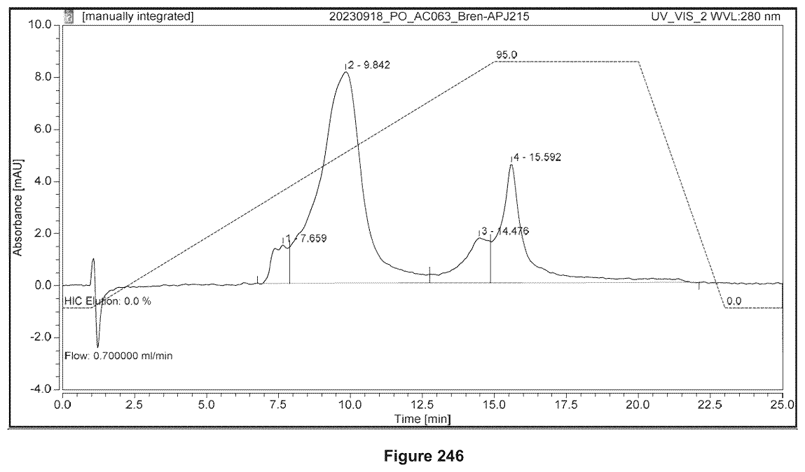

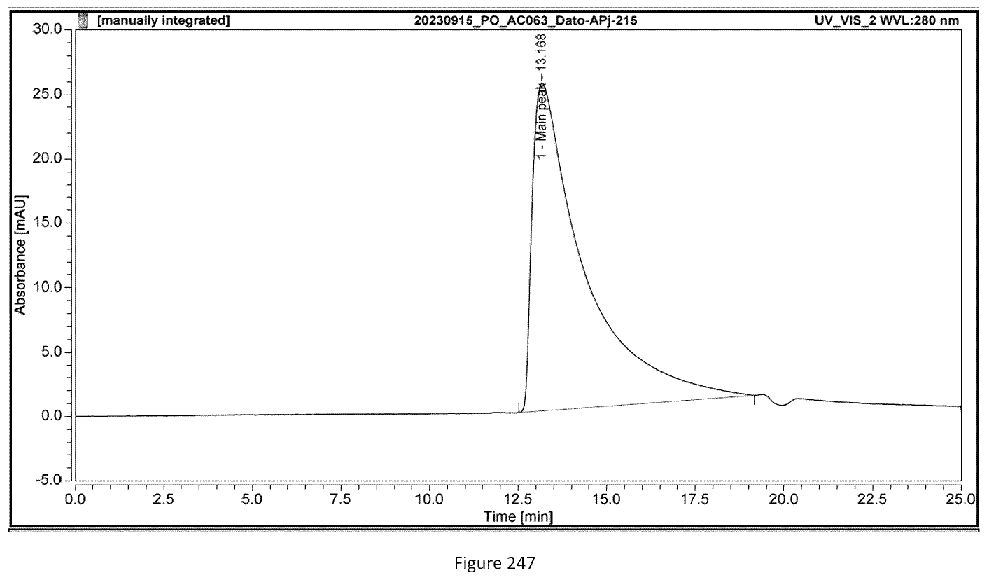

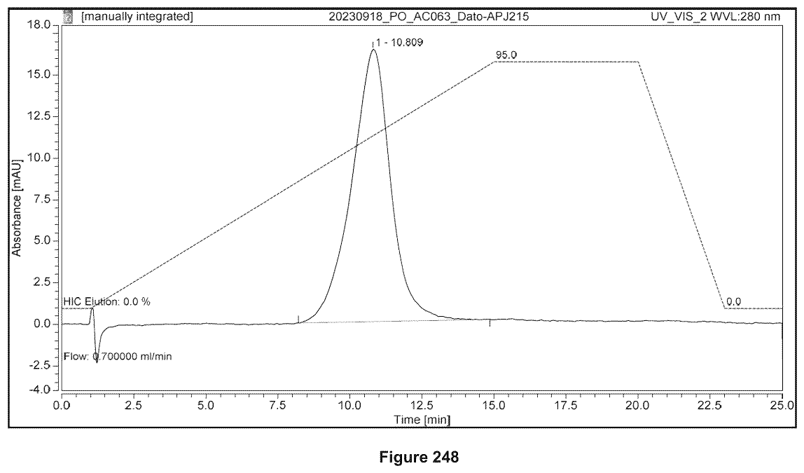

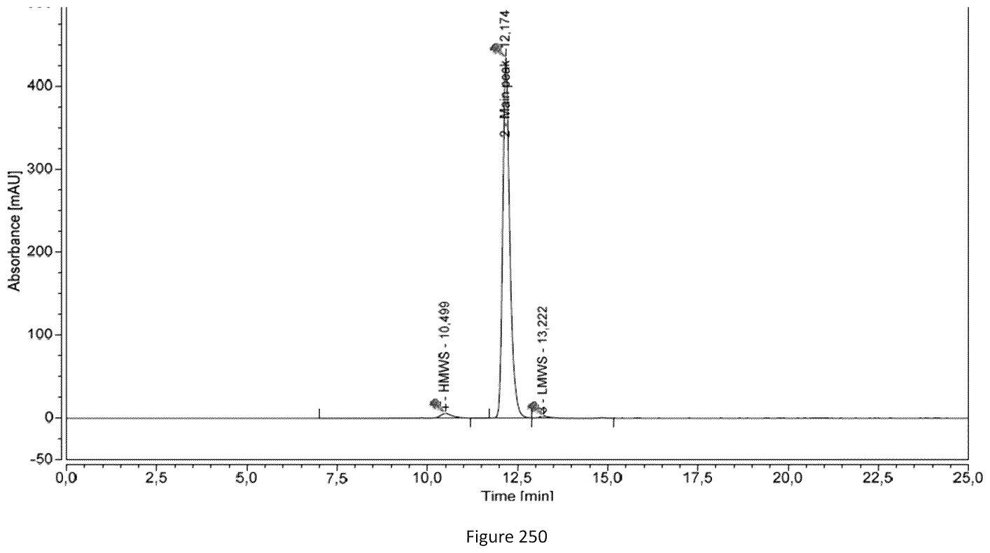

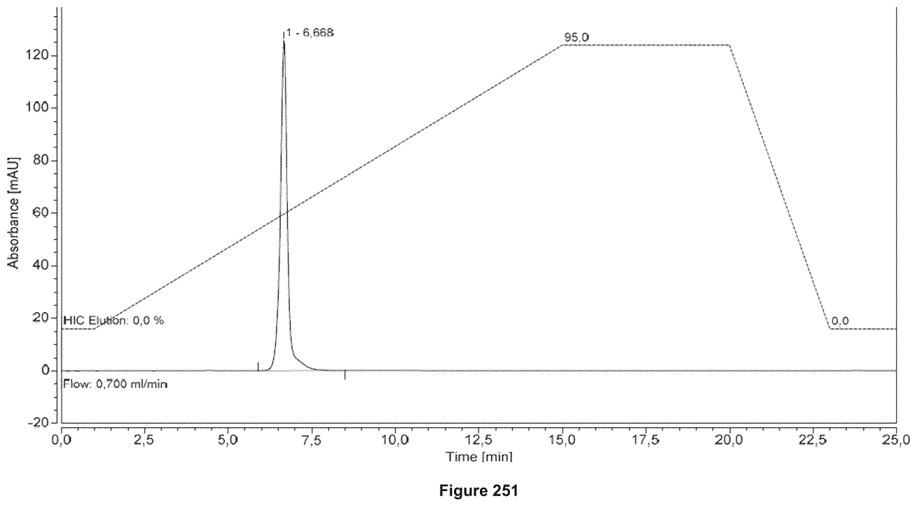

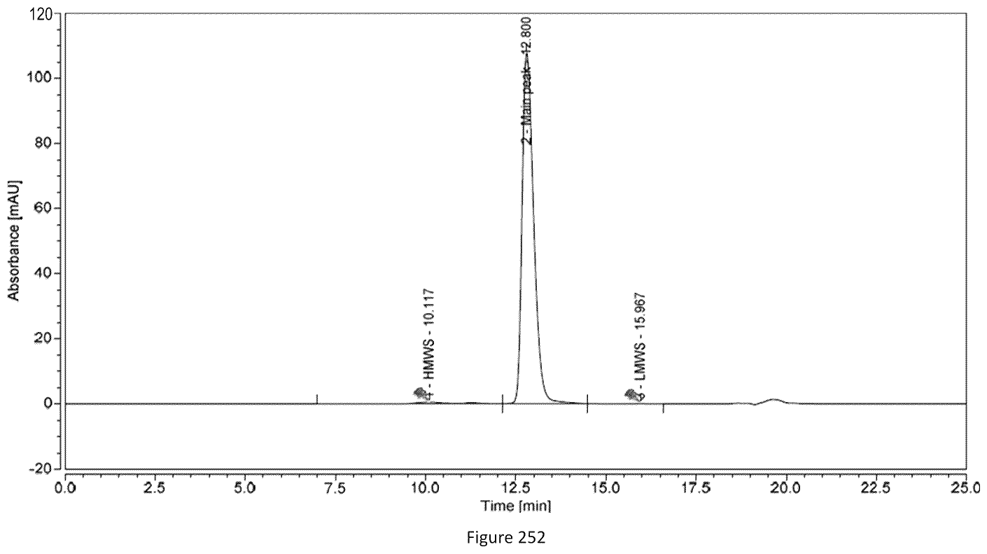

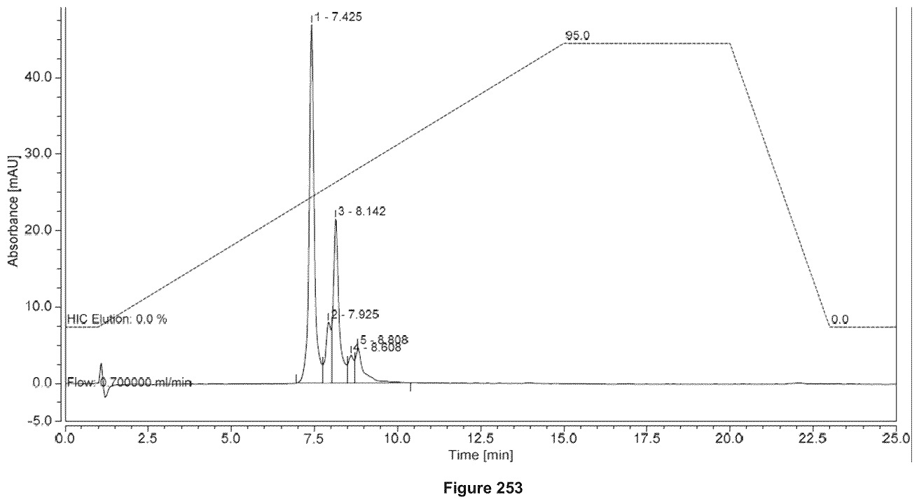

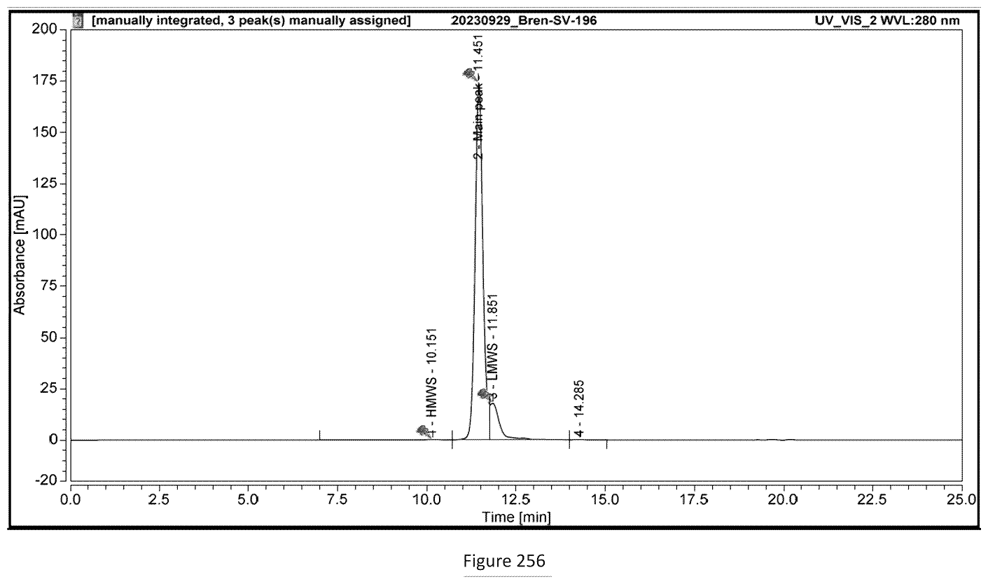

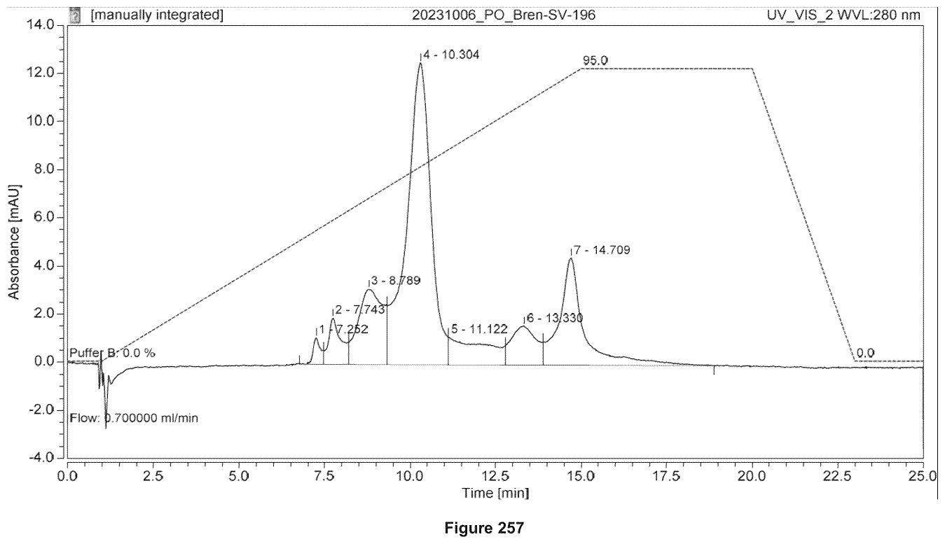

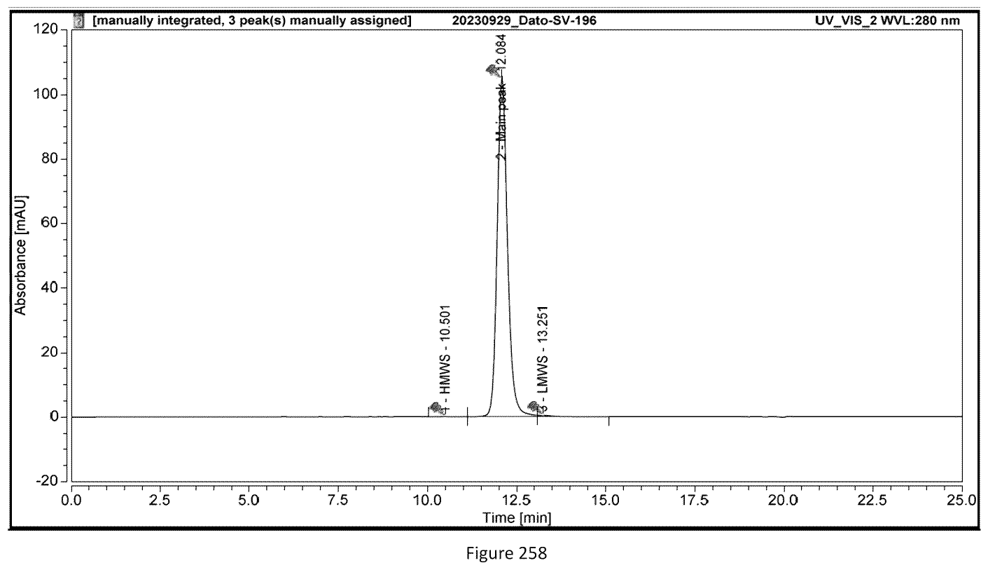

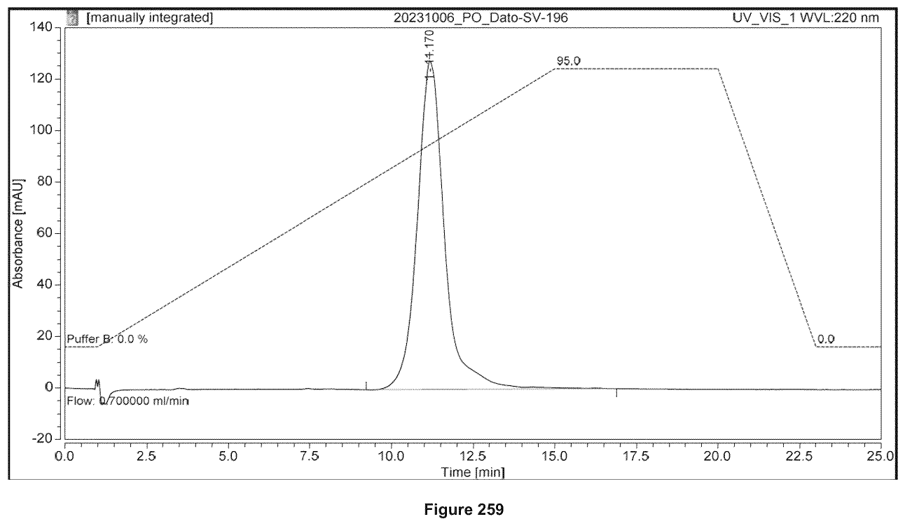

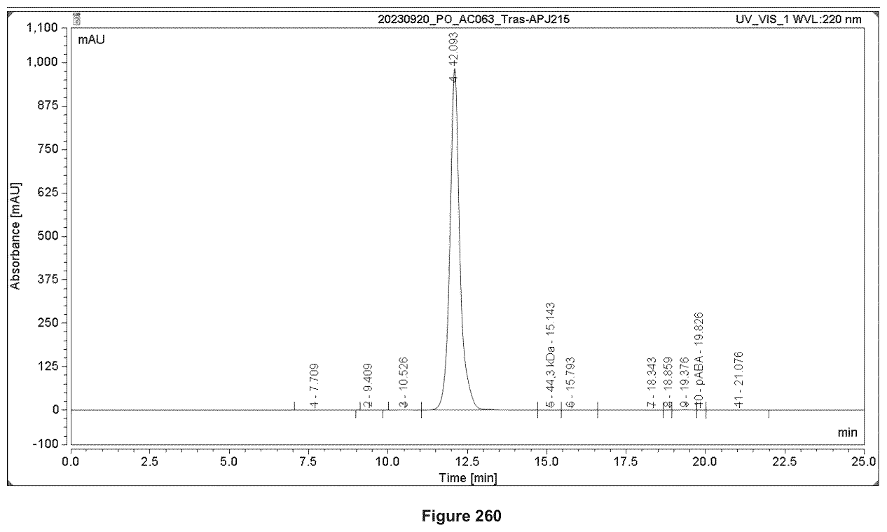

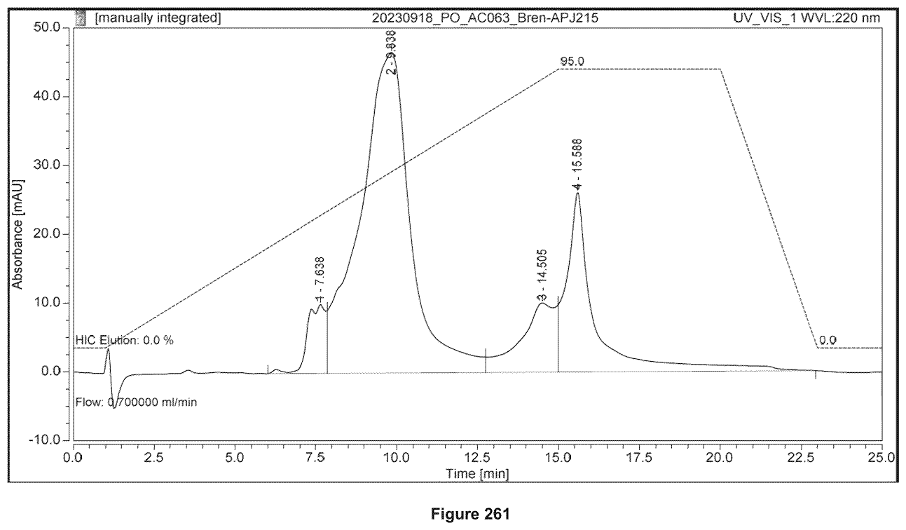

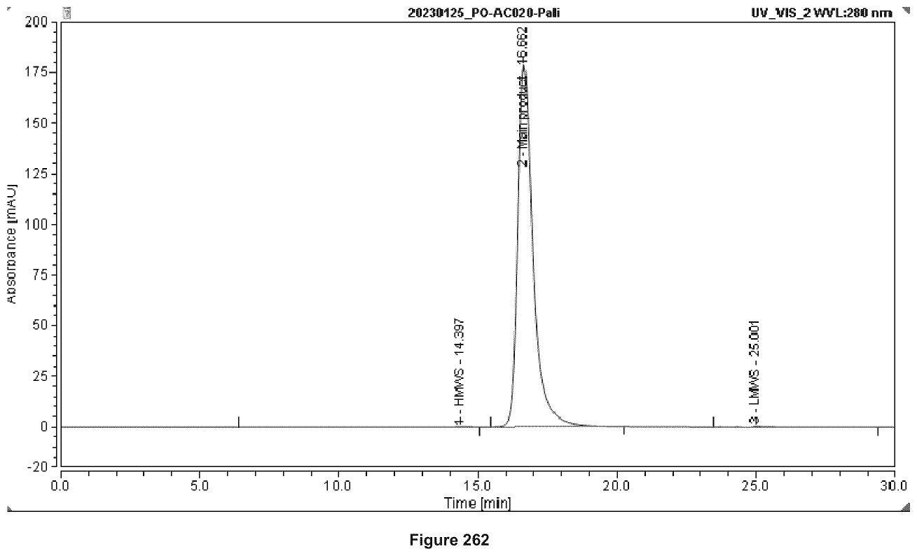

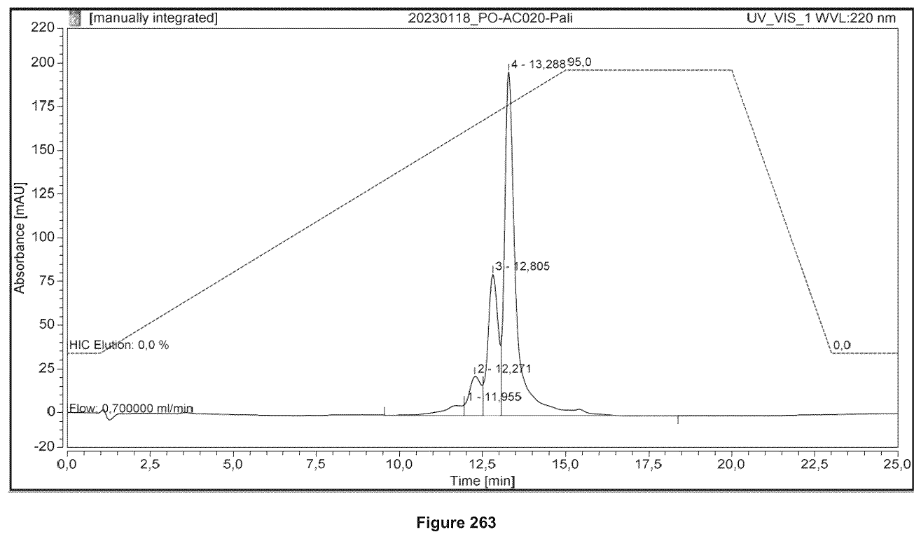

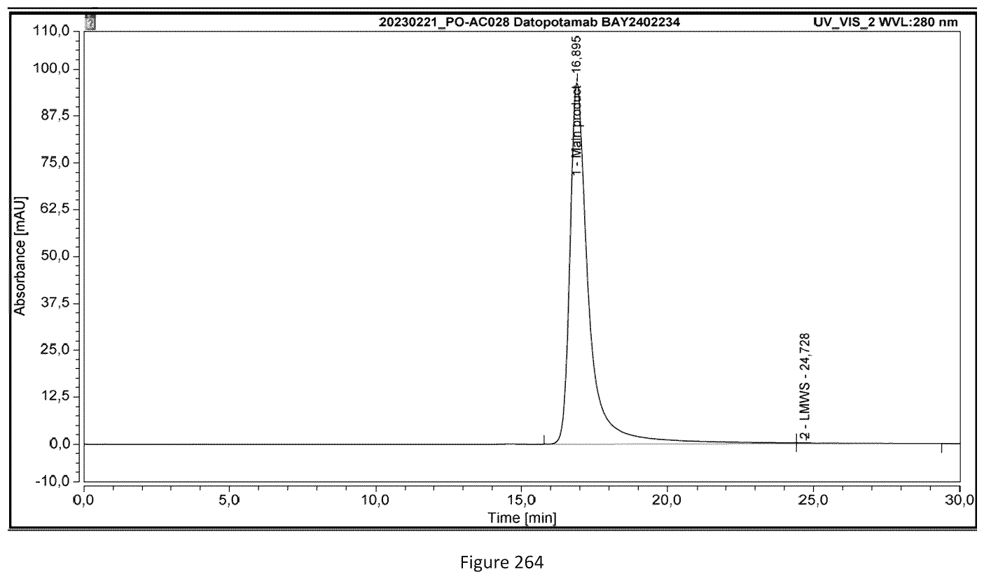

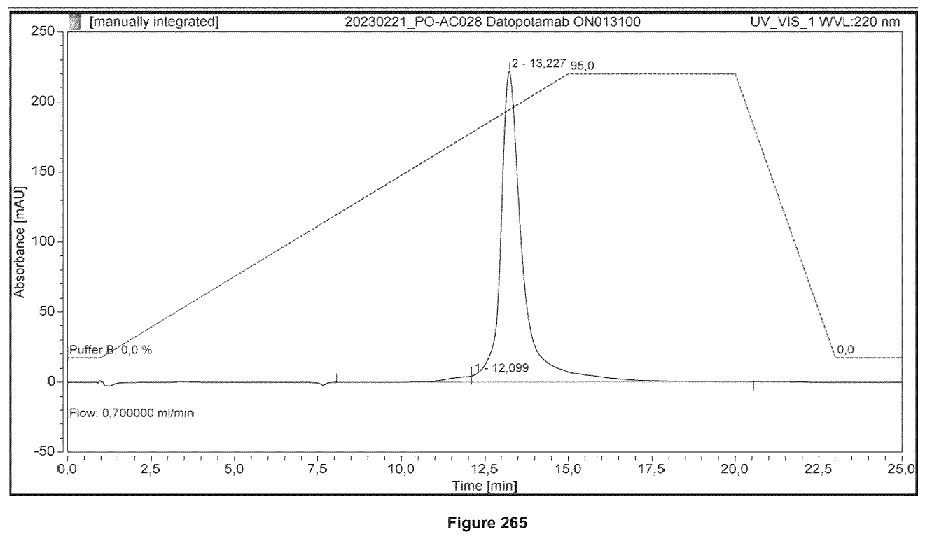

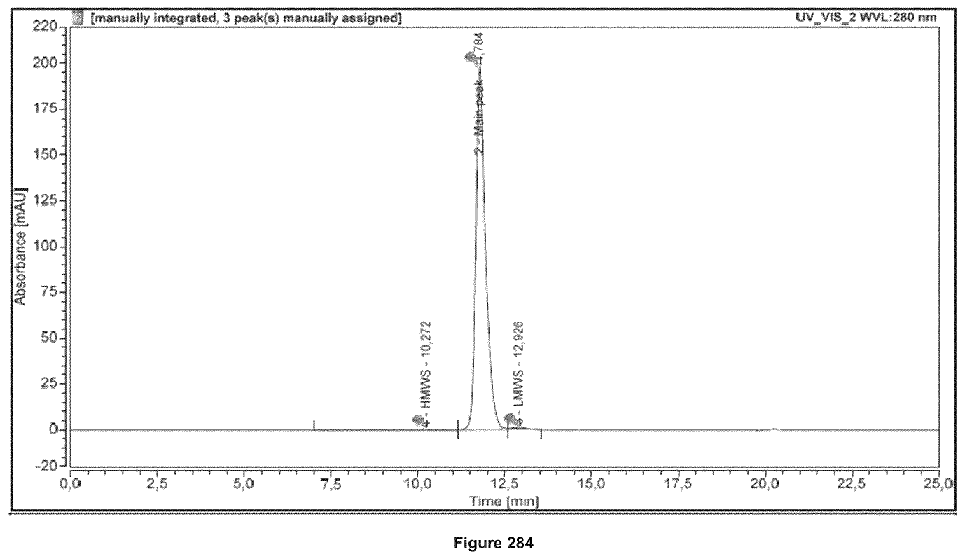

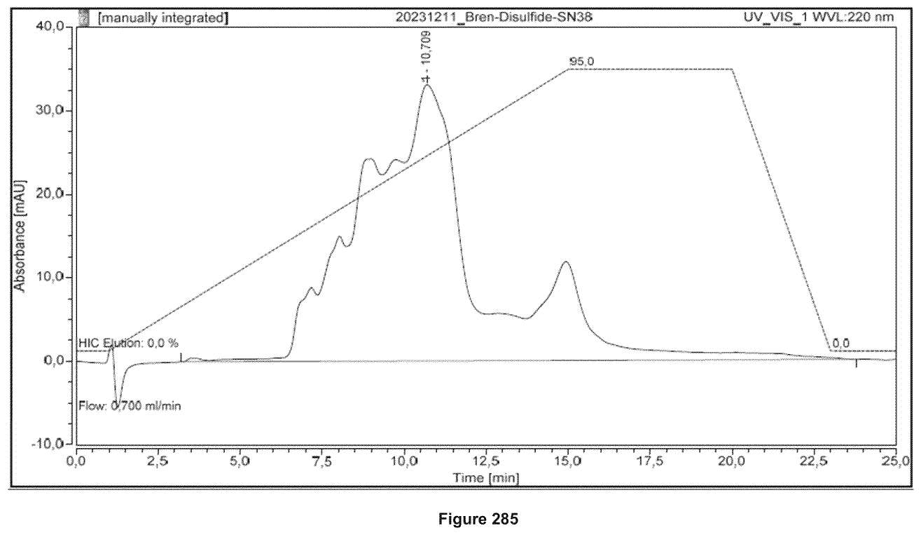

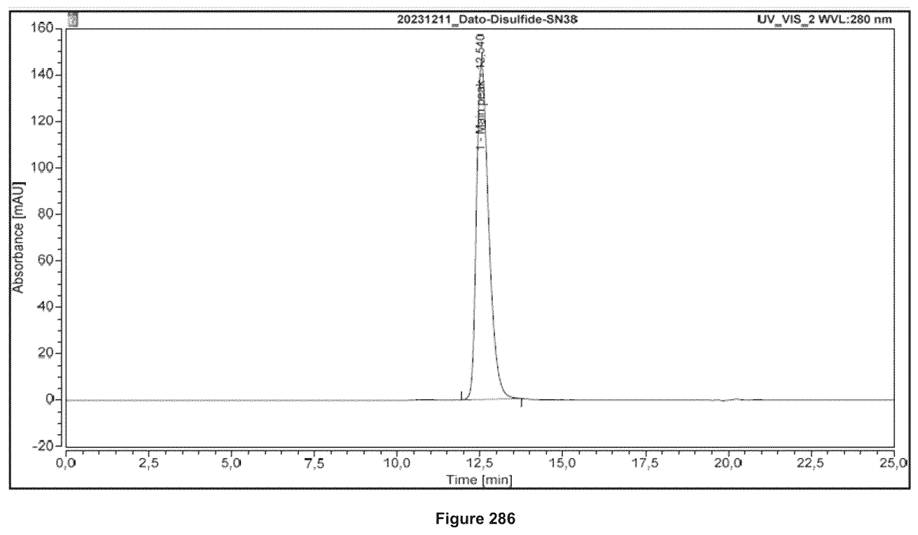

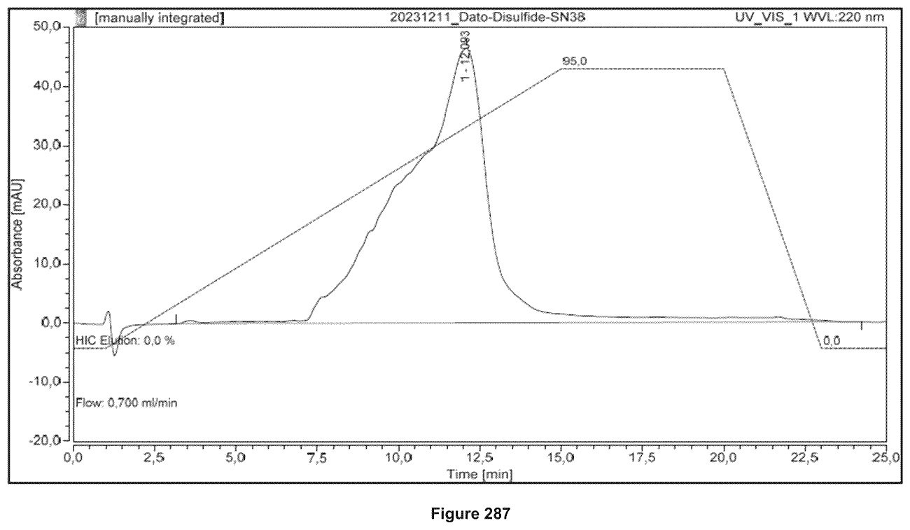

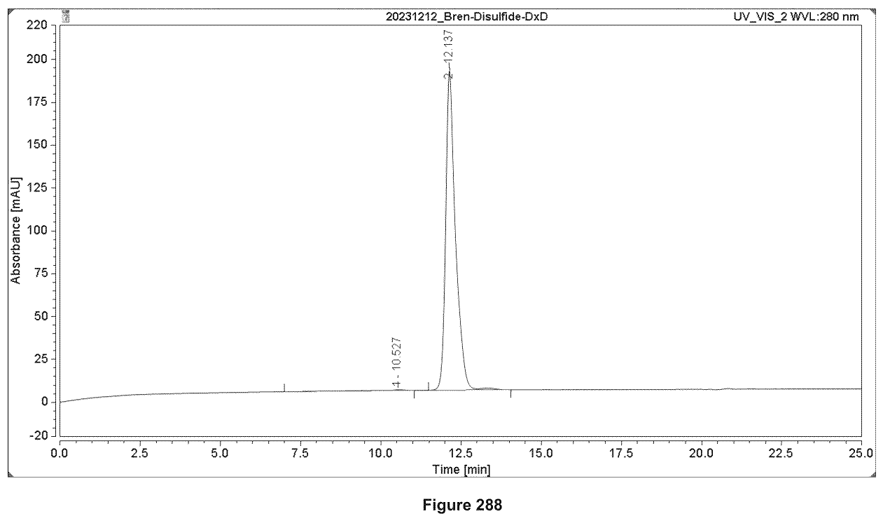

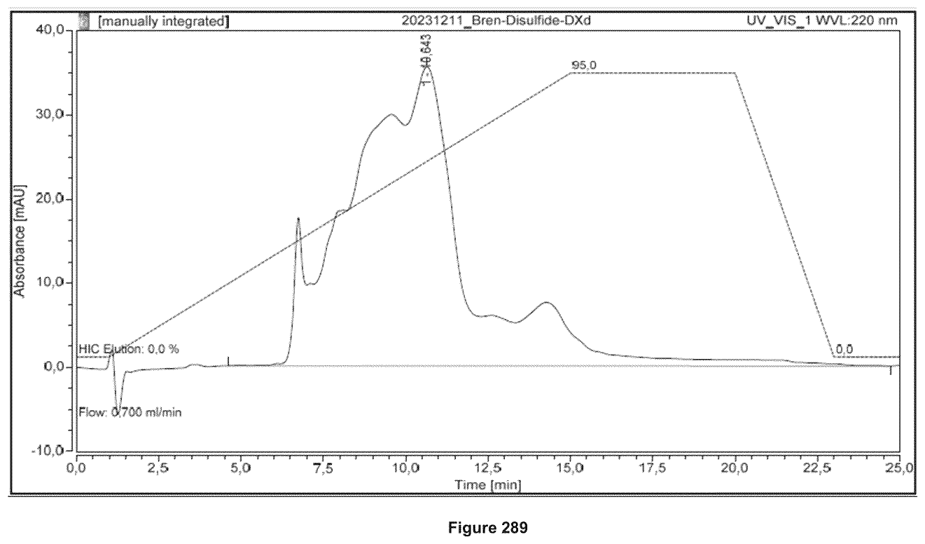

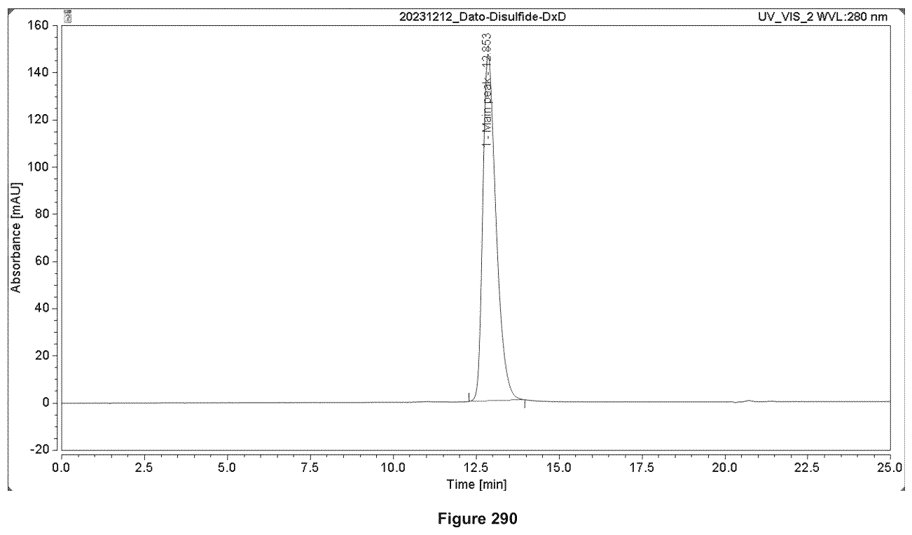

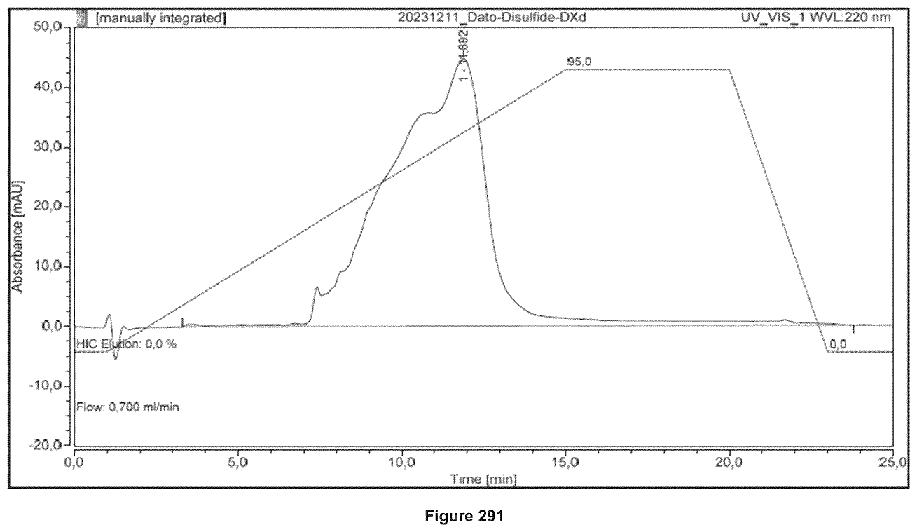

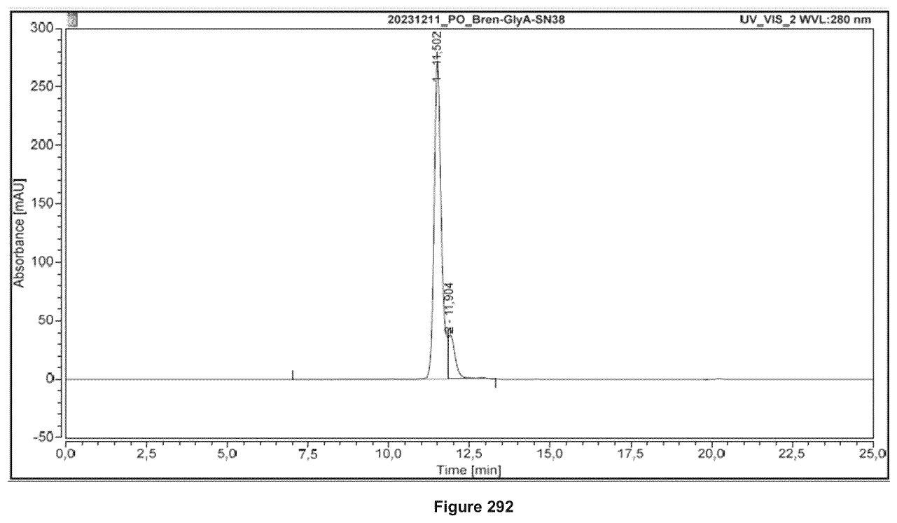

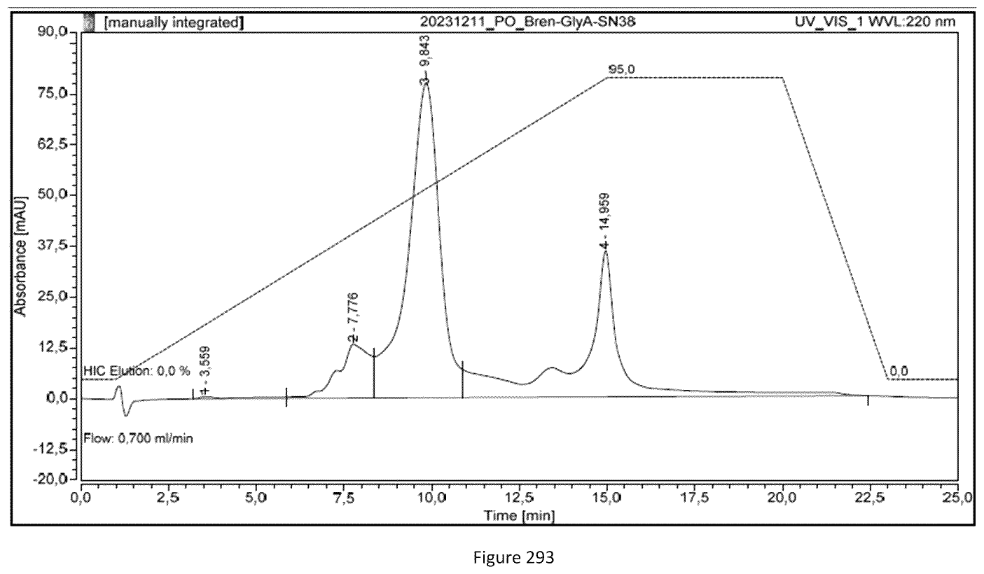

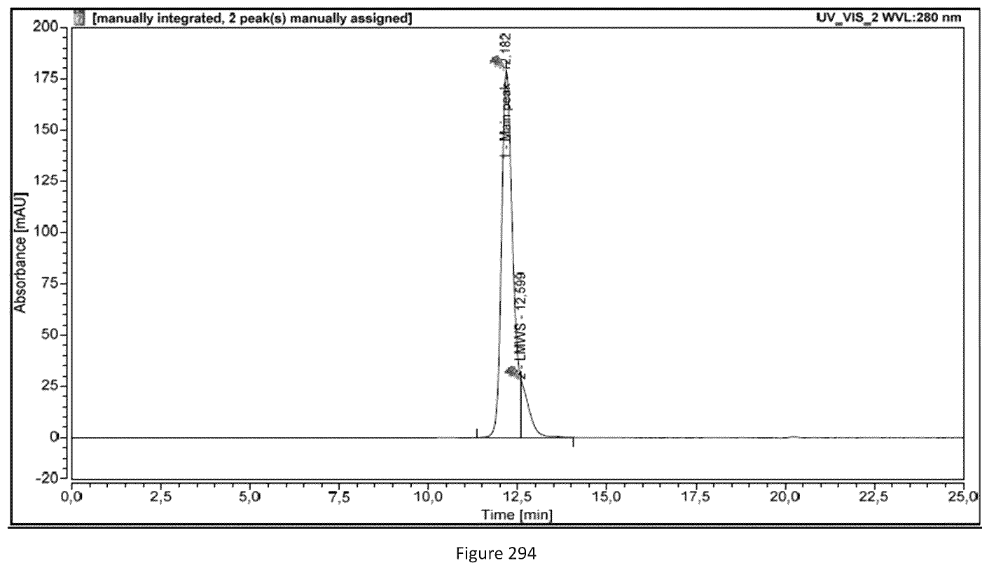

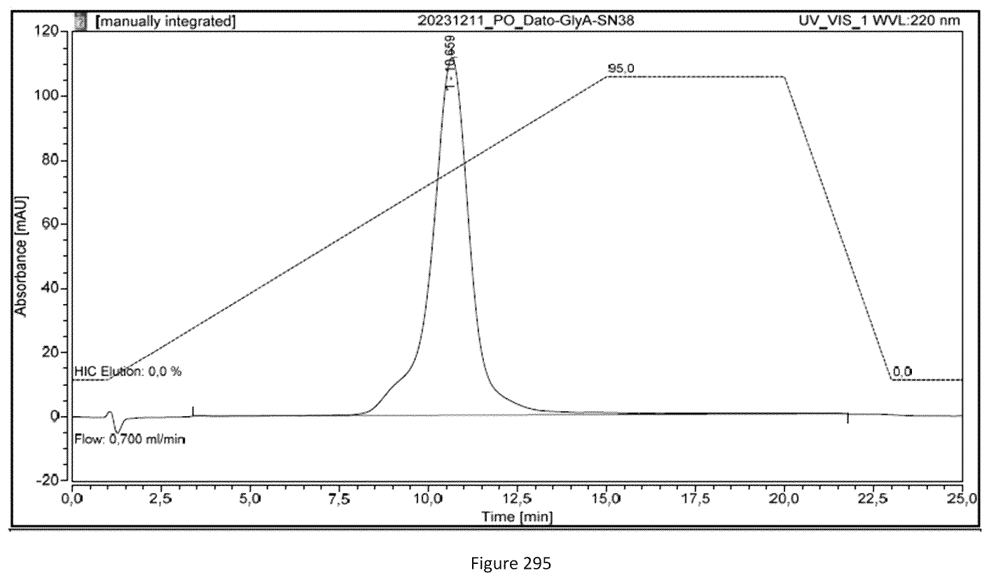

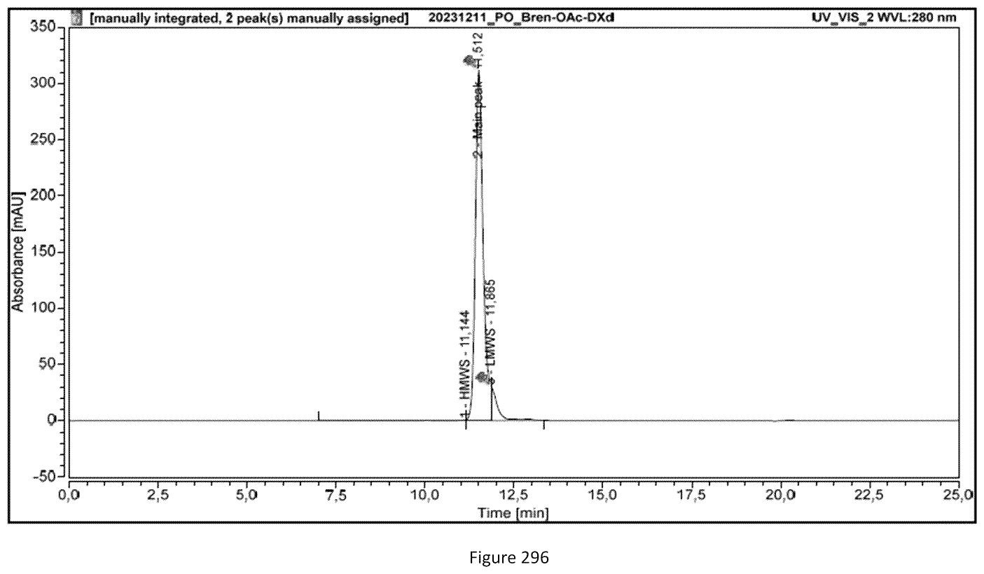

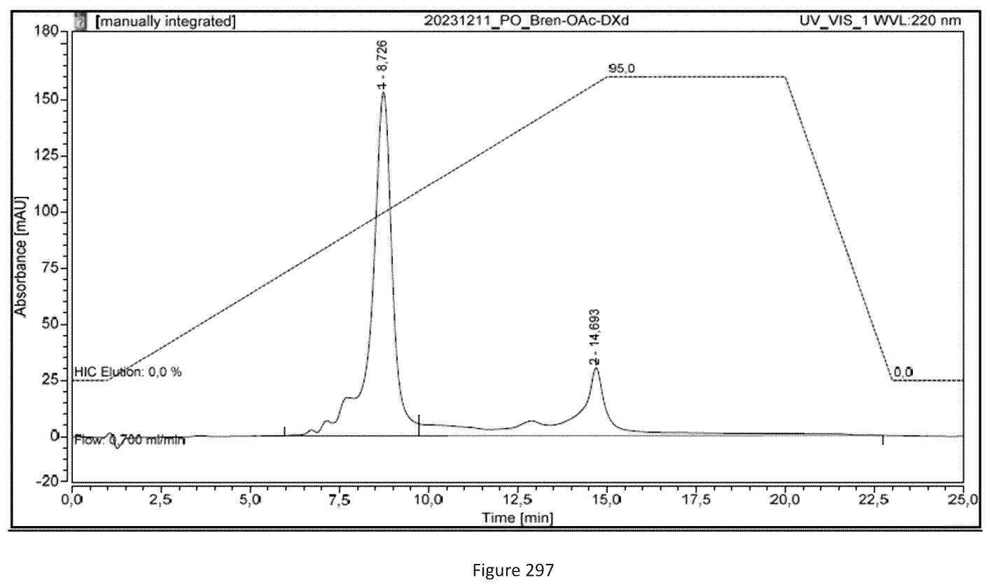

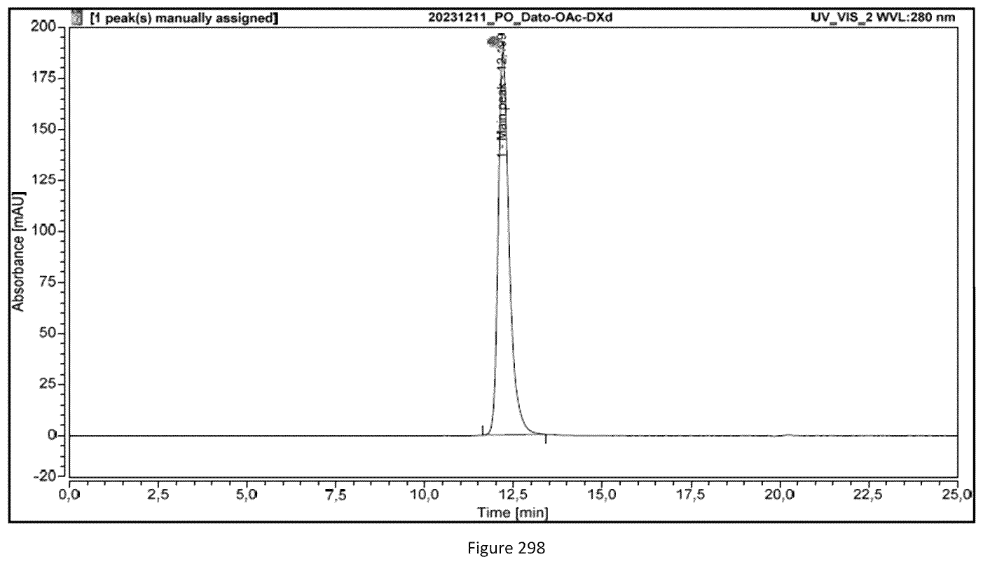

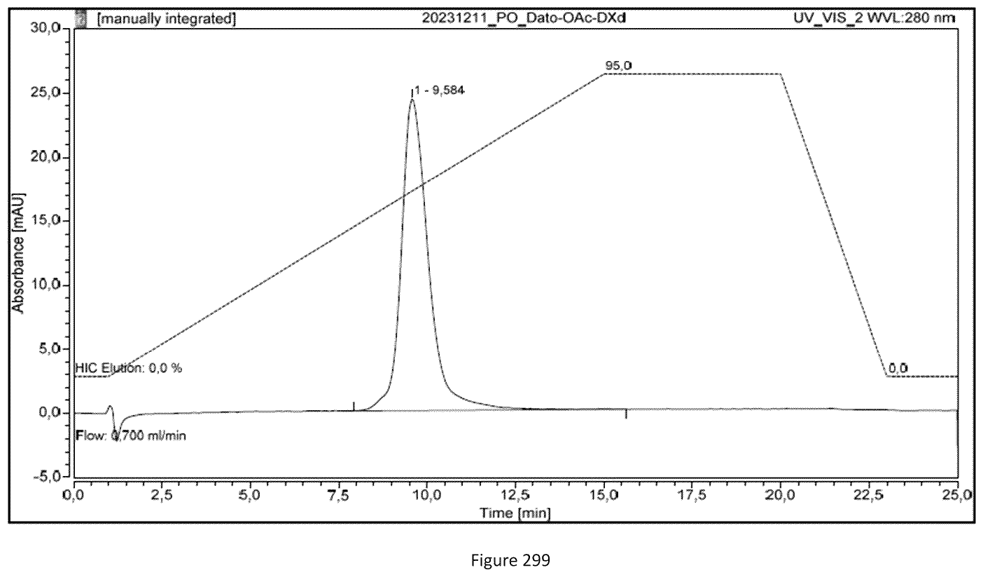

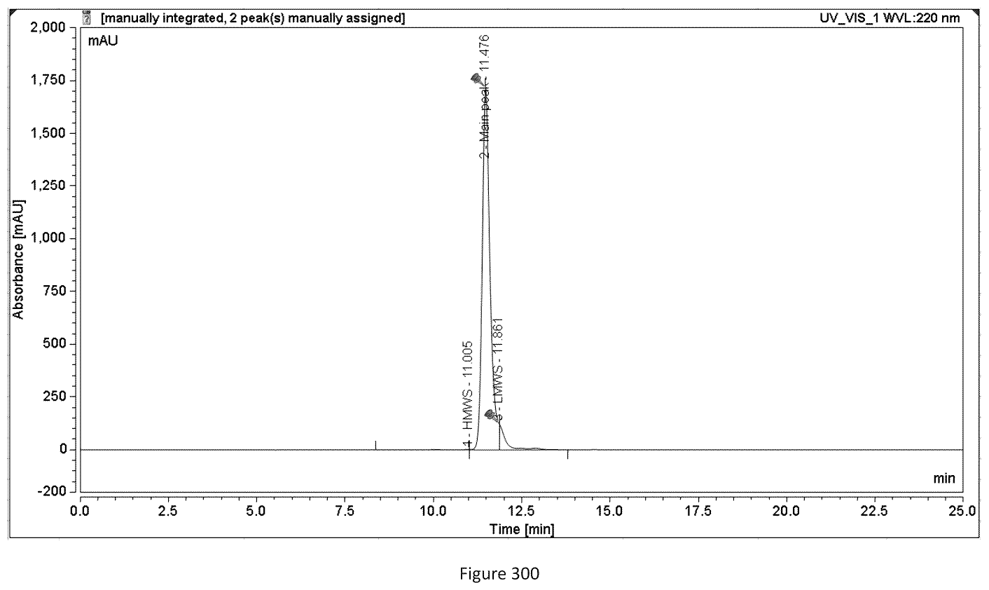

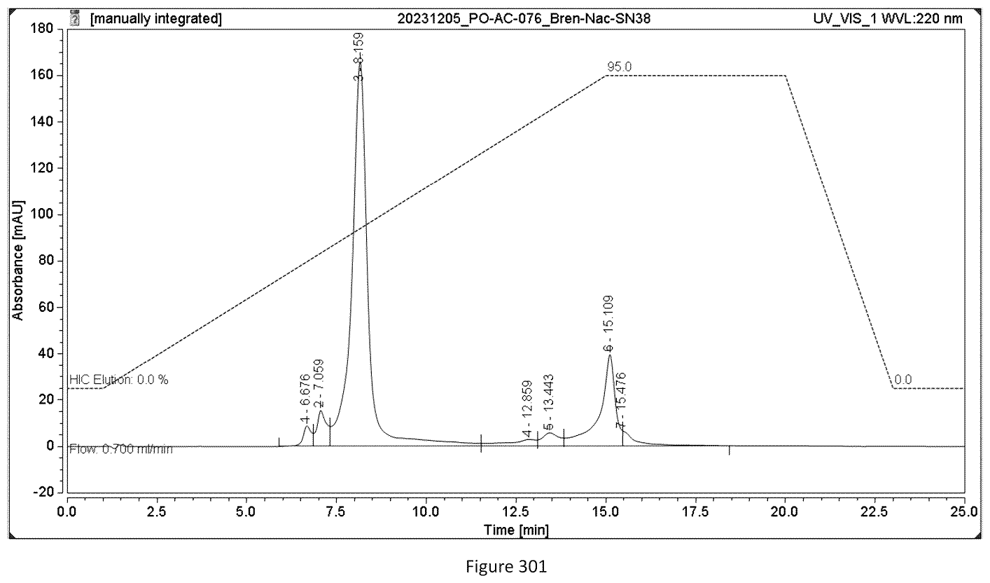

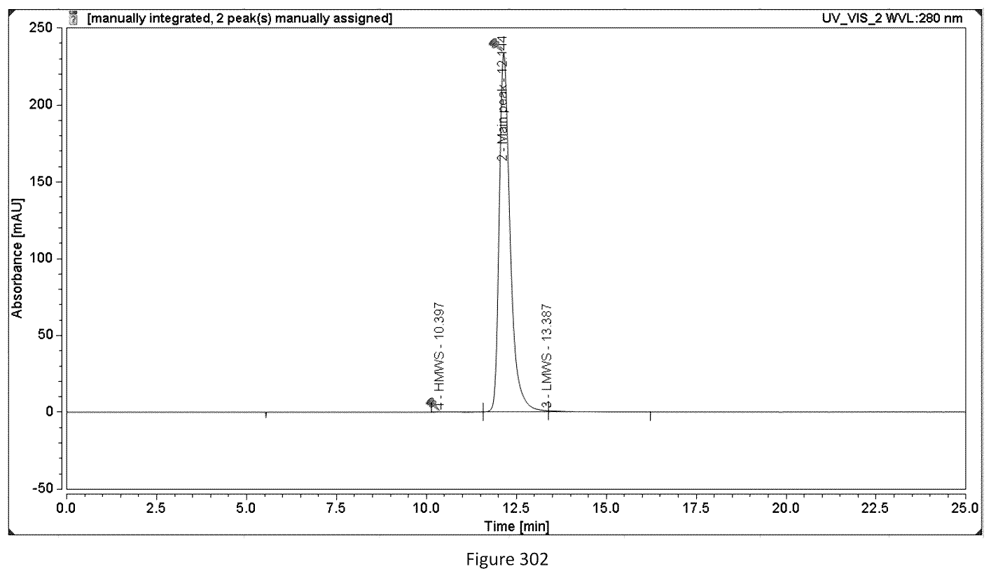

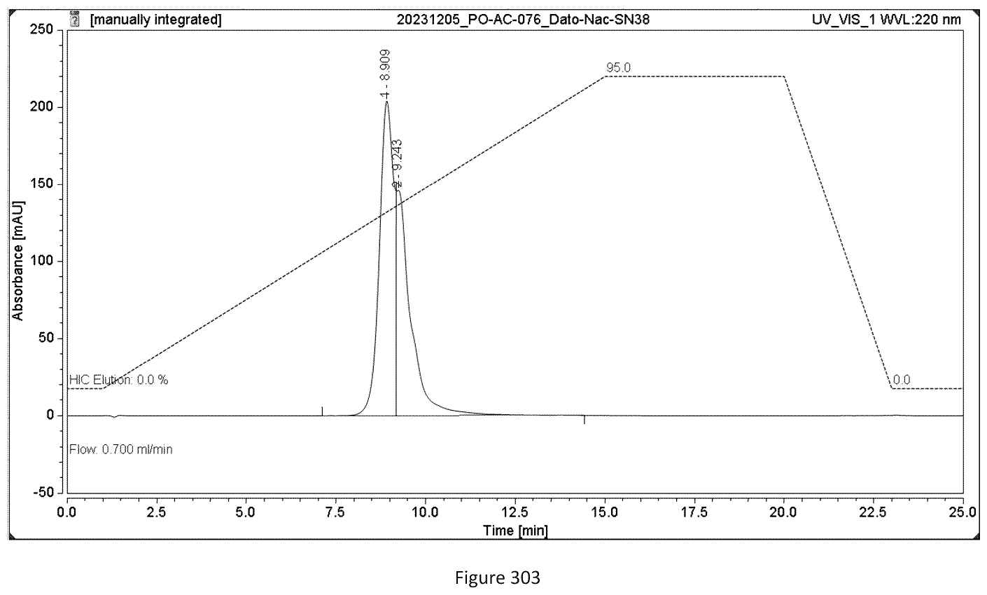

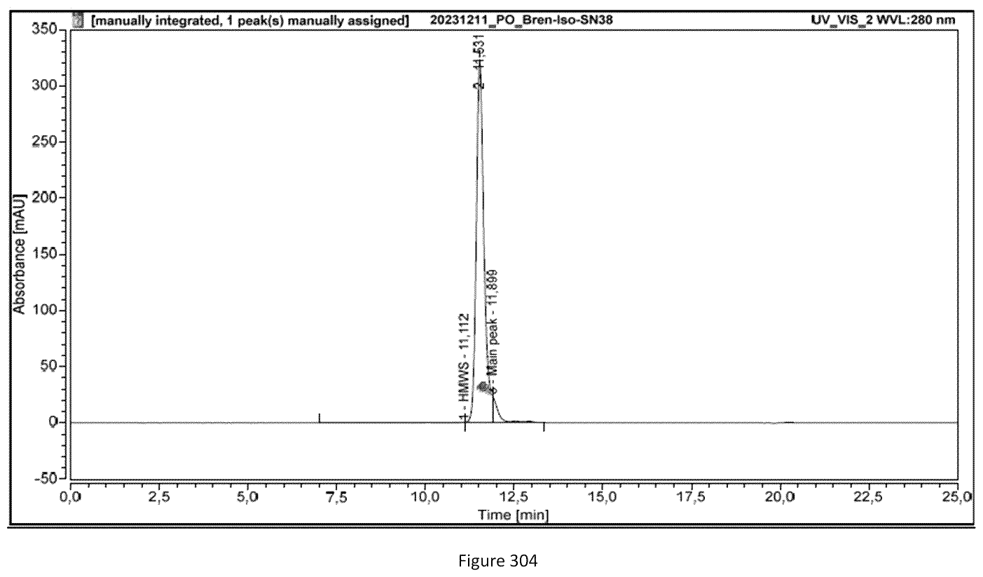

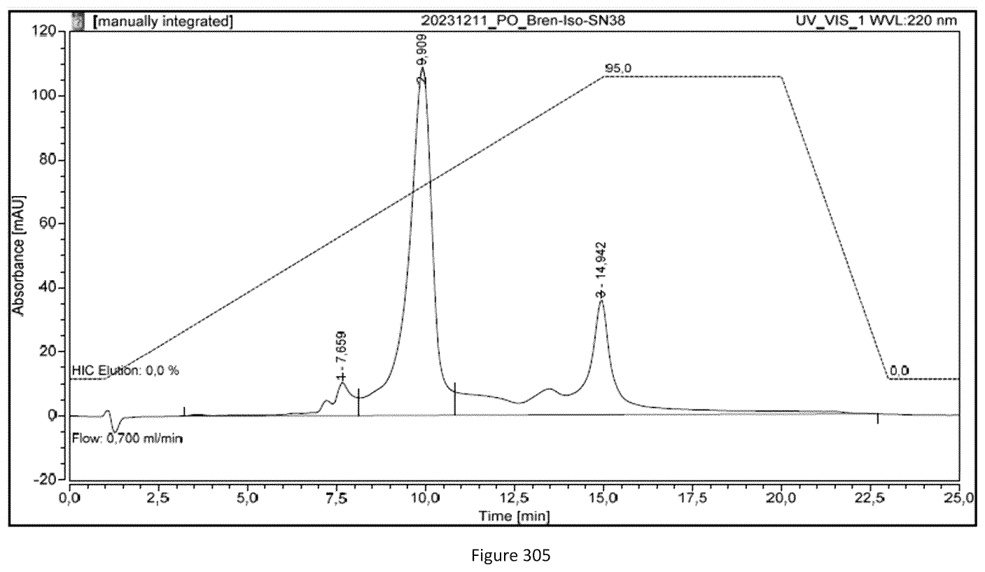

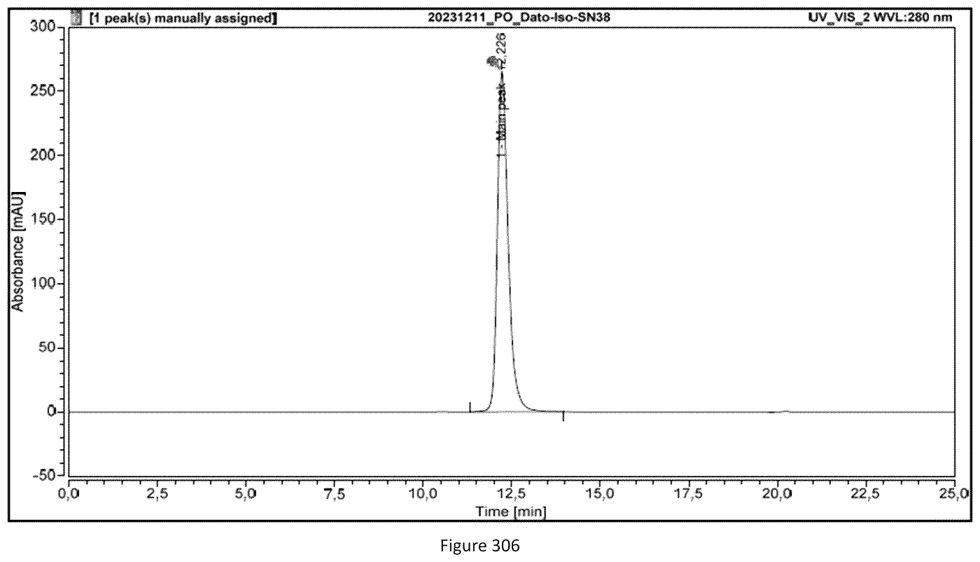

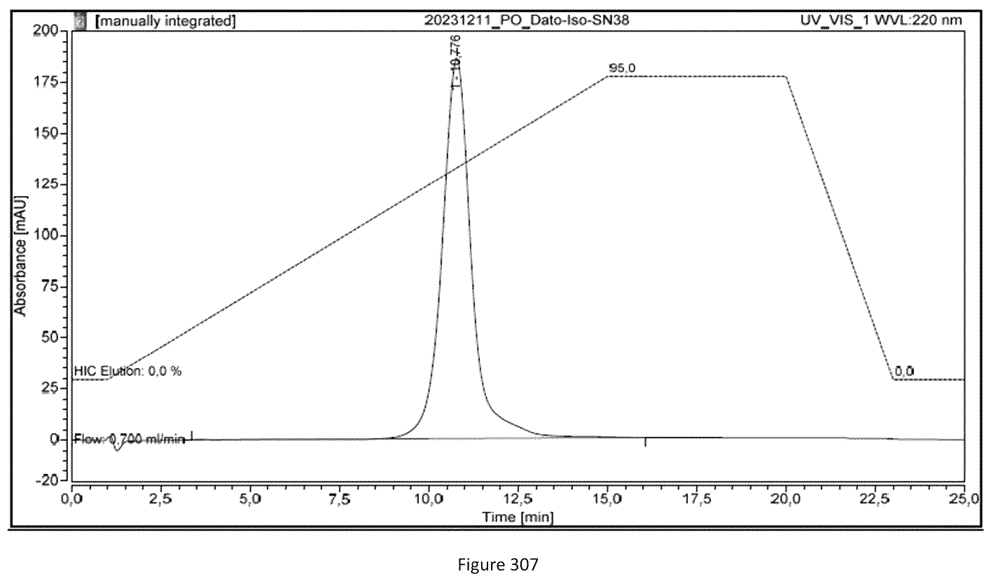

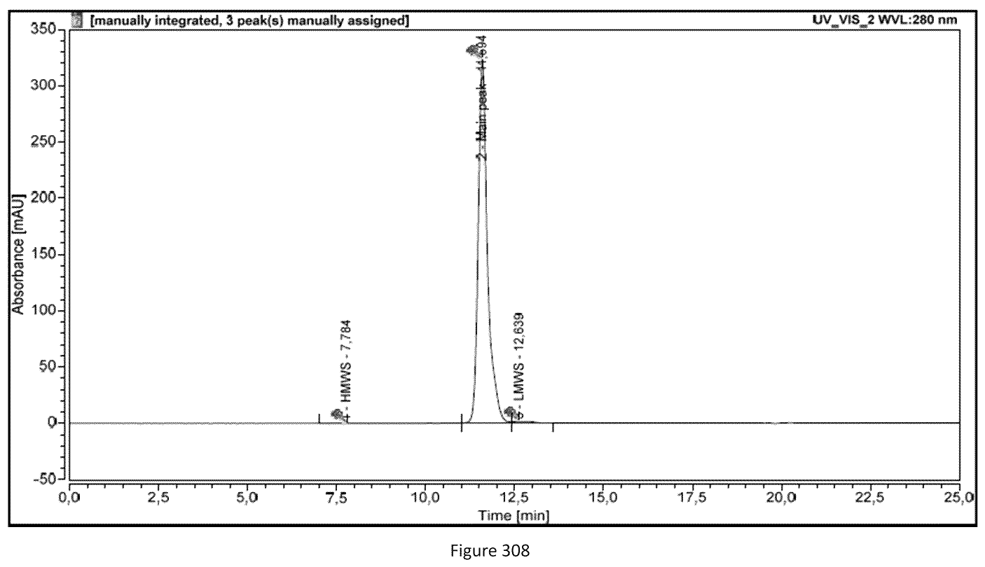

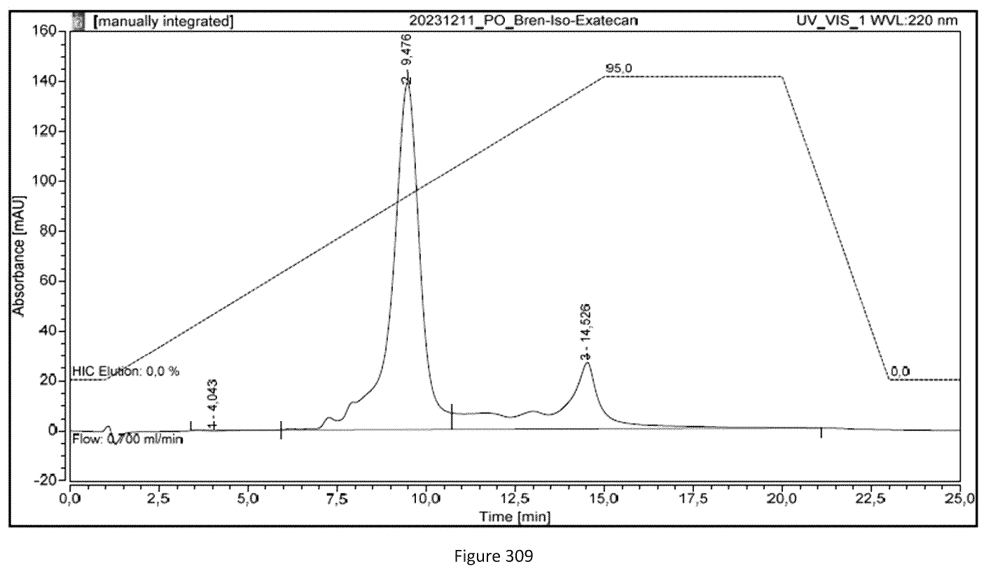

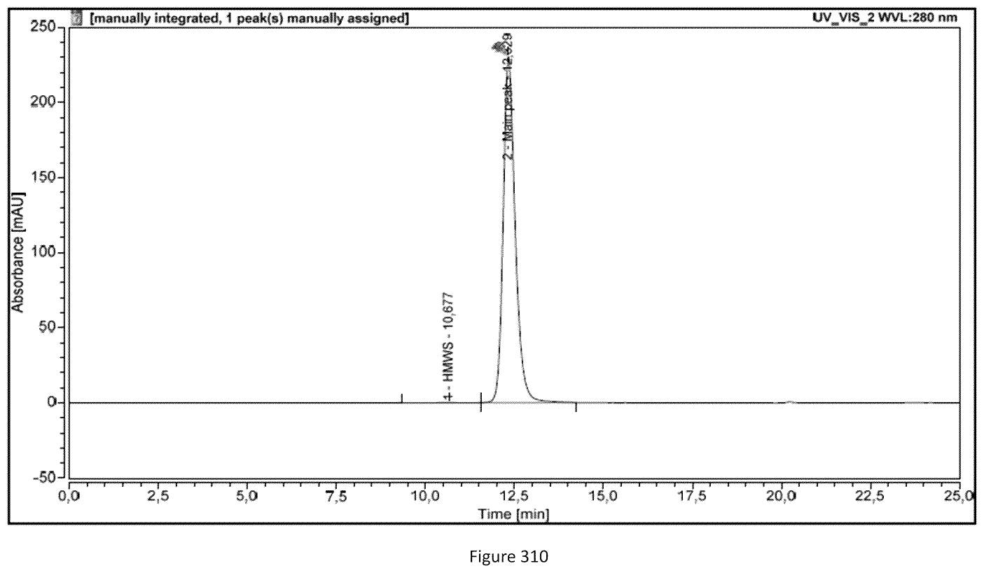

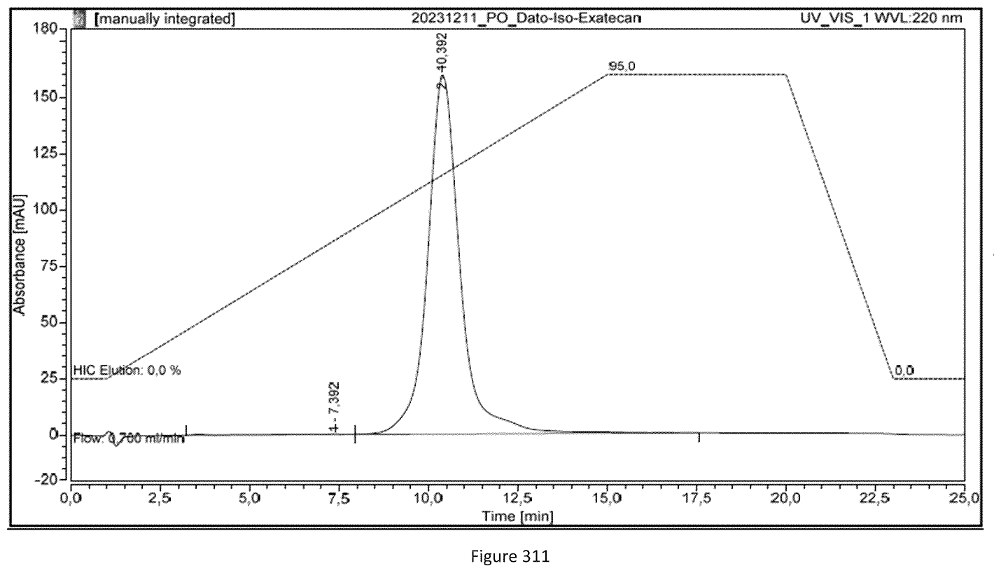

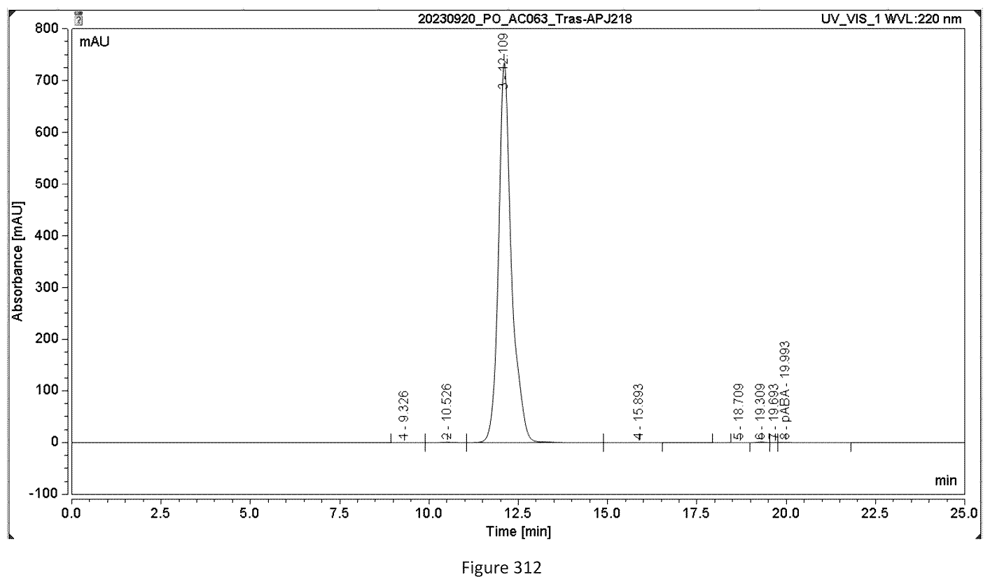

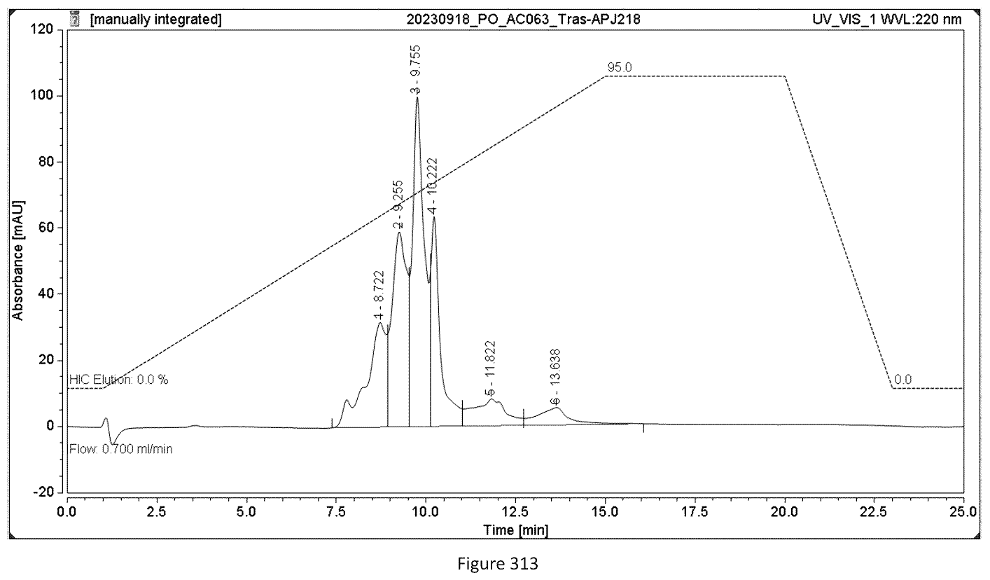

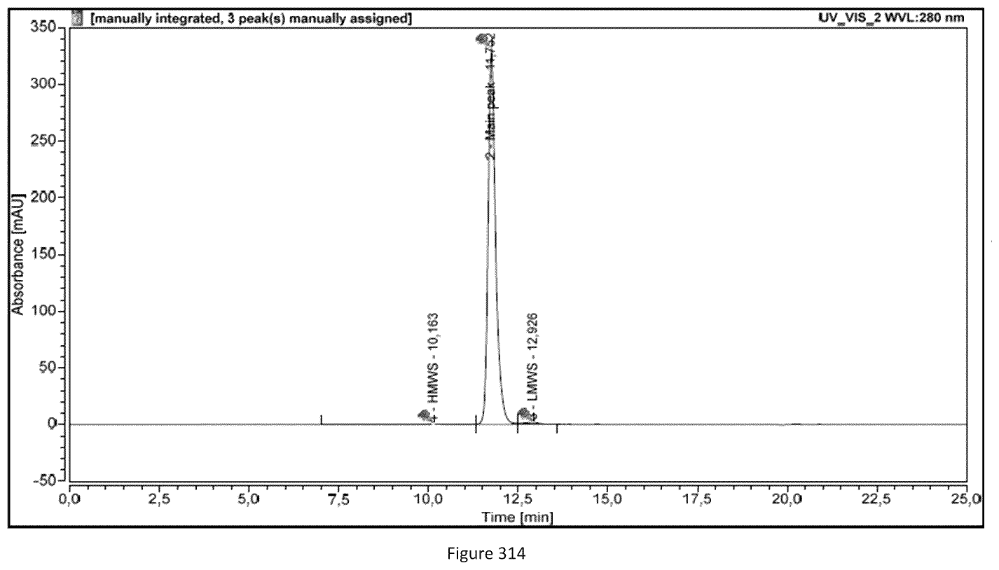

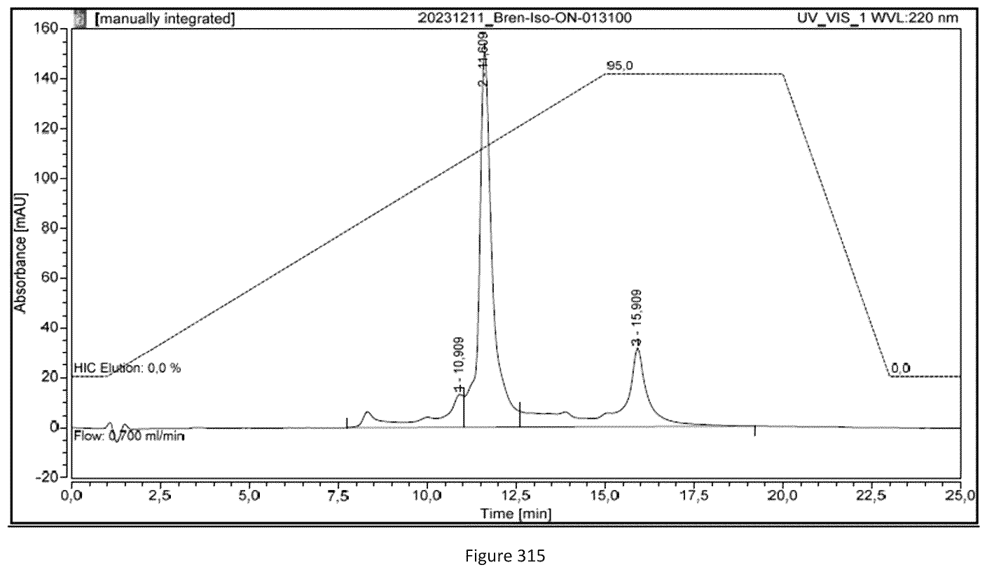

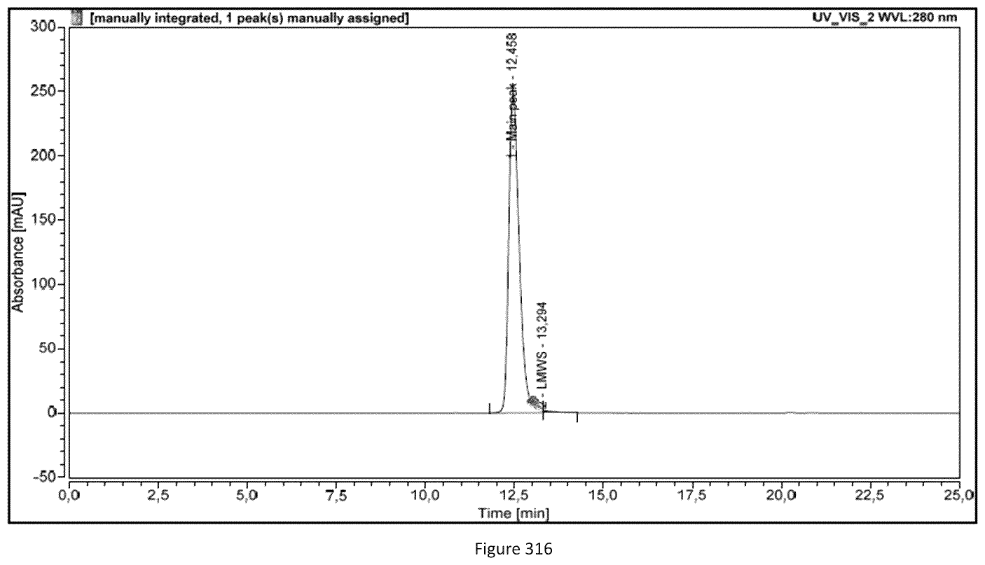

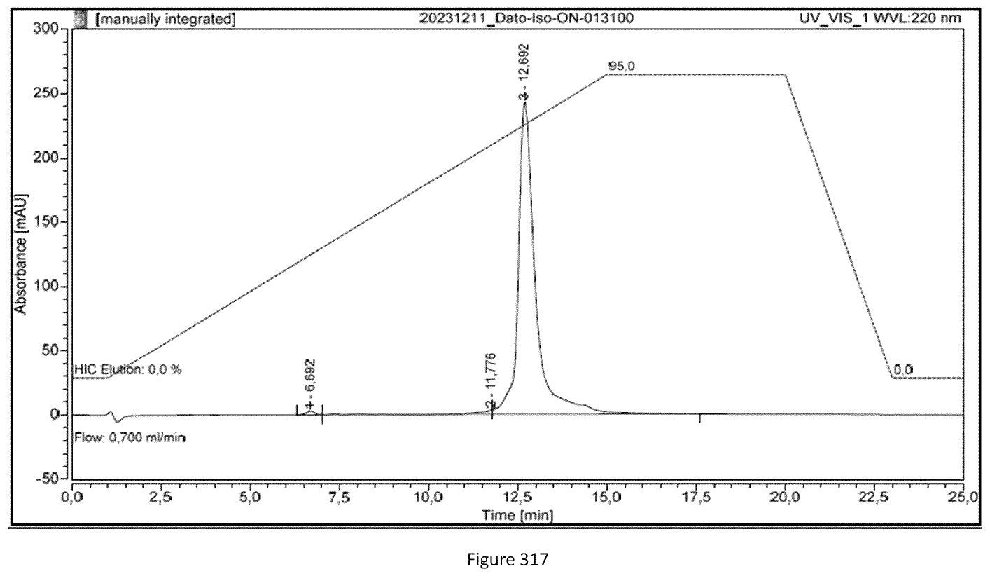

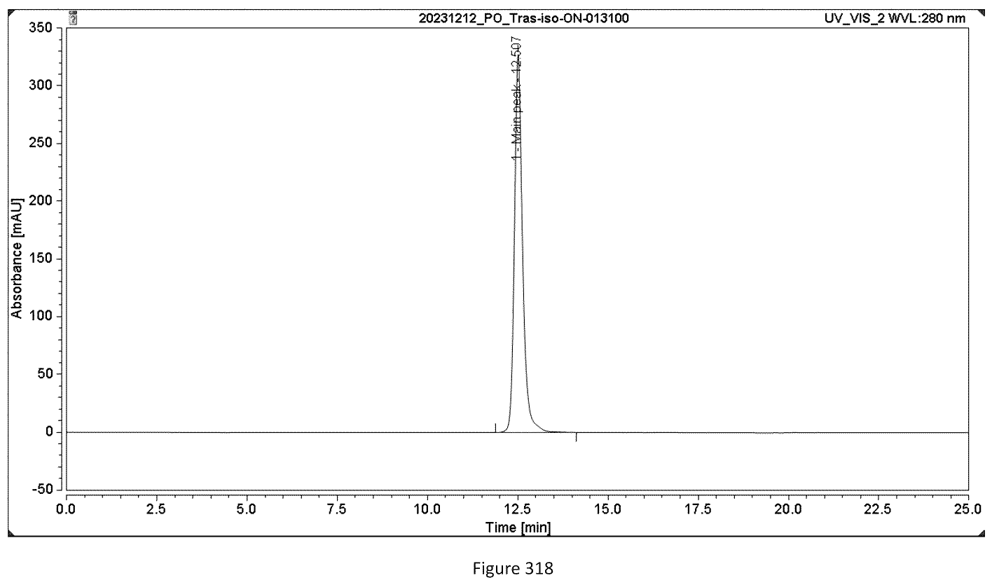

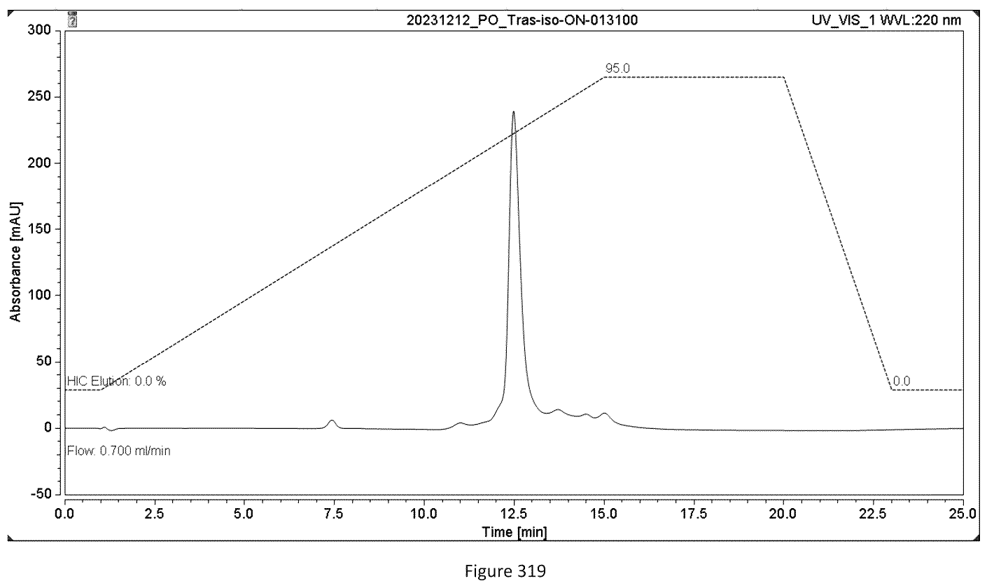

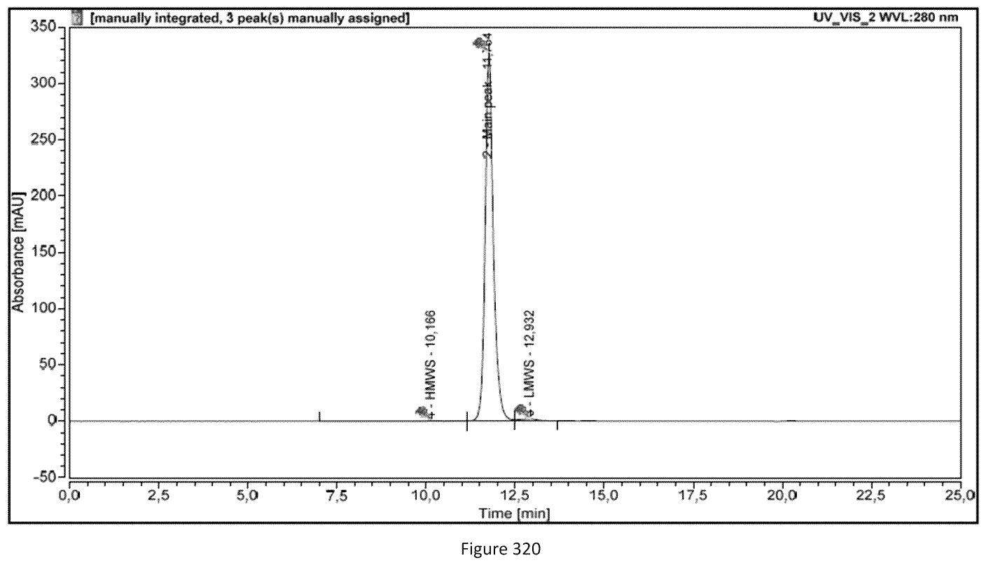

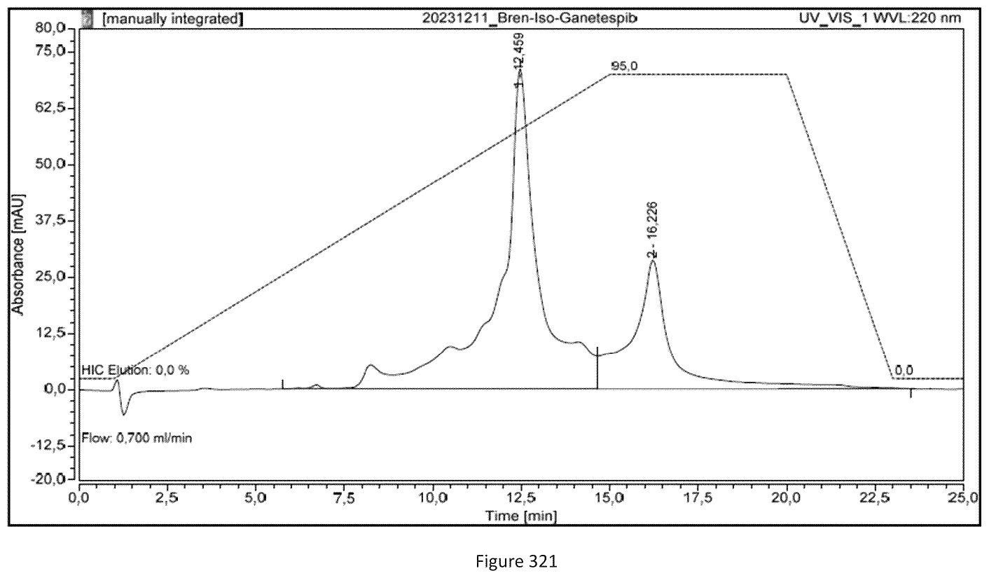

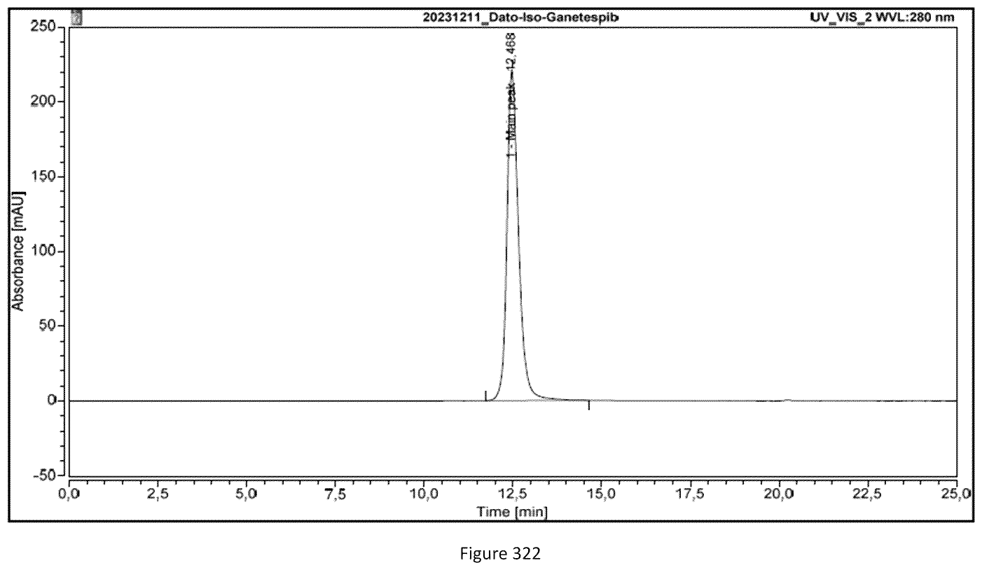

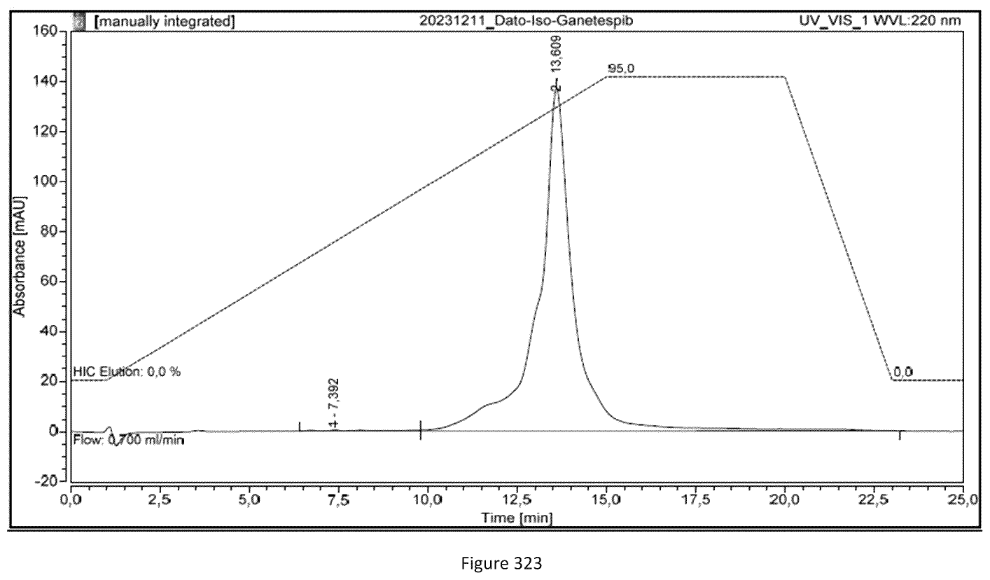

### Example 2: Analytics of the Synthesized ADCs and Antibody Starting Materials

| | | | MS analysis |
|---|---|---|---|
| | | | of the unconjugated |
| | | Analytical | mAb of fully conjugated |
| Antibody/ADC | Analytical SEC | HIC | DAR8 species, DAR |
| [symbol] | see FIG. 21 | see FIG. 22 | LC: calcd.: 23439 found: 23438 HC: calcd.: 49146 found: 49149 |
| Trastuzumab | | | |
| [symbol] | see FIG. 23 | see FIG. 24 | LC: calcd.: 23335 found: 23334 HC: calcd.: 49288 found: 49289 |
| Sacituzumab | | | |
| [symbol] | see FIG. 25 | see FIG. 26 | LC: calcd.: 23296 found: 23294 HC: calcd.: 49200 found: 49202 |
| Palivizumab | | | |
| 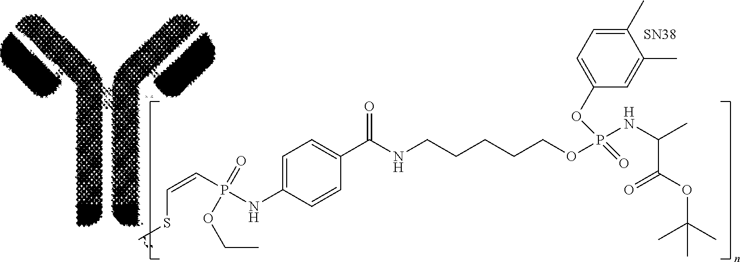 | | | DARav: 7.8 LC: calcd.: 24358 found: 24358 HC: calcd.: 51904 found: 51909 |
| Trastuzumab-O-P5(OEt)-amidopentyl-Phosphoramidate-N-(L-alanine-tert.-butylester)-O-SN38 | | | |
| 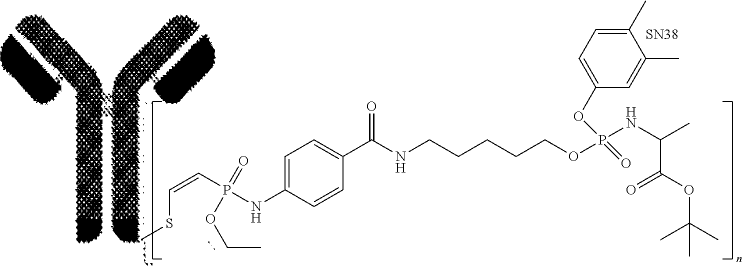 | | | DARav: 7.8 LC: calcd.: 24254 found: 24254 HC: calcd.: 52046 found: 52049 |
| Sacituzumab-O-P5(OEt)-amidopentyl-Phosphoramidate-N-(L-alanine-tert.-butylester)-O-SN38 | | | |
| 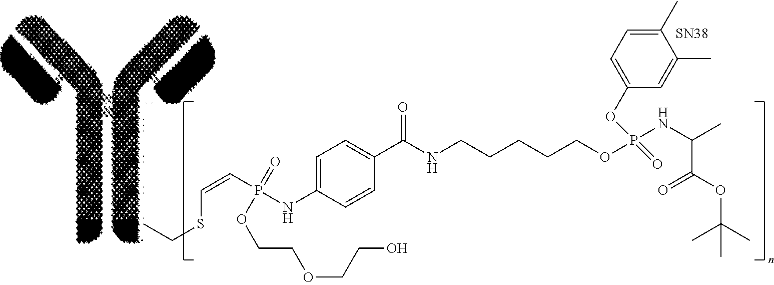 | see FIG. 27 | see FIG. 28 | DARav: 8.0 LC: calcd.: 24314 found: 24314 HC: calcd.: 52226 found: 52229 |
| Sacituzumab-O-P5(PEG2)-amidopentyl-Phosphoramidate-N-(L-alanine-tert.-butylester)-O-SN38 | | | |
| 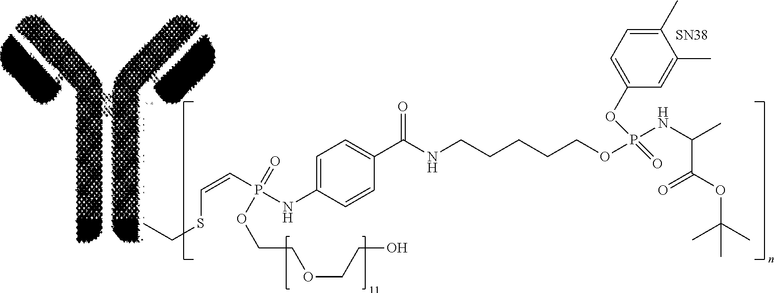 | | | DARav: 8.0 LC: calcd.: 24858 found: 24858 HC: calcd.: 53405 found: 53411 |
| Trastuzumab-O-P5(PEG12)-amidopentyl-Phosphoramidate-N-(L-alanine-tert.-butylester)-O-SN38 | | | |
| 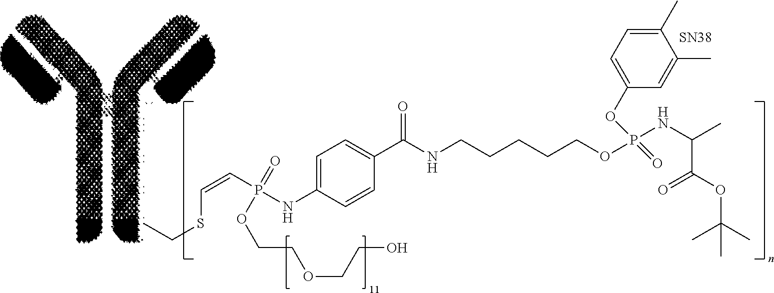 | see FIG. 29 | see FIG. 30 | DARav: 8.0 LC: calcd.: 24755 found: 24754 HC: calcd.: 53547 found: 53550 |
| Sacituzumab-O-P5(PEG12)-amidopentyl-Phosphoramidate-N-(L-alanine-tert.-butylester)-O-SN38 | | | |
| 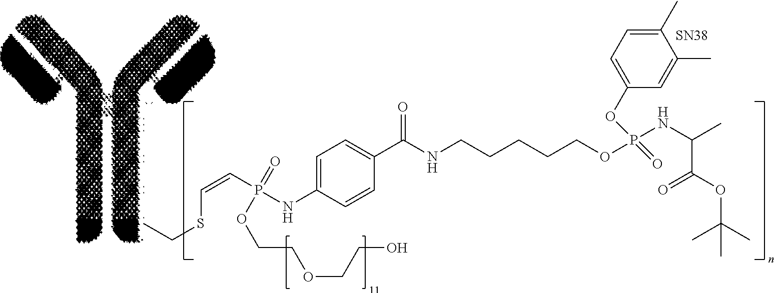 | see FIG. 31 | see FIG. 32 | DARav: 8.0 LC: calcd.: 25300 found: 25299 HC: calcd.: 53605 found: 53605 |
| Palivizumab-O-P5(PEG12)-amidopentyl-Phosphoramidate-N-(L-alanine-tert.butylester)-O-SN38 | | | |
| 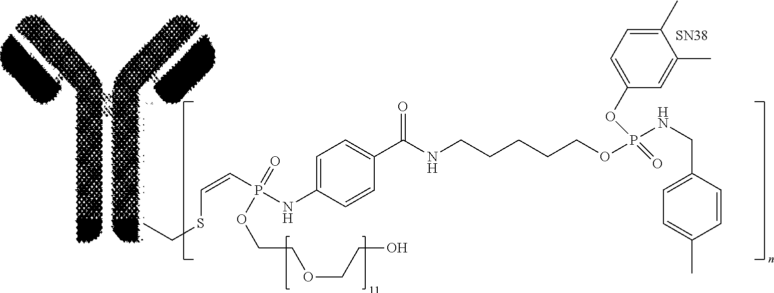 | | | DARav: 8.0 LC: calcd.: 24835 found: 24834 HC: calcd.: 53333 found: 53338 |
| Trastuzumab-O-P5(PEG12)-amidopentyl-Phosphoramidate-N-(4-Methylbenzyl)-O-SN38 | | | |
| 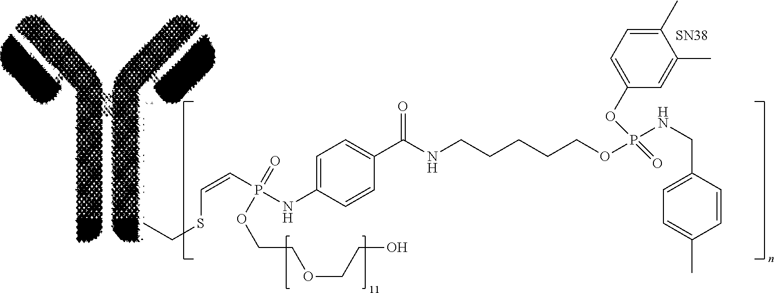 | | | DARav: 8.0 LC: calcd.: 24692 found: 24692 HC: calcd.: 53475 found: 53479 |
| Sacituzumab-O-P5(PEG12)-amidopentyl-Phosphoramidate-N-(4-Methylbenzyl)-O-SN38 | | | |
| 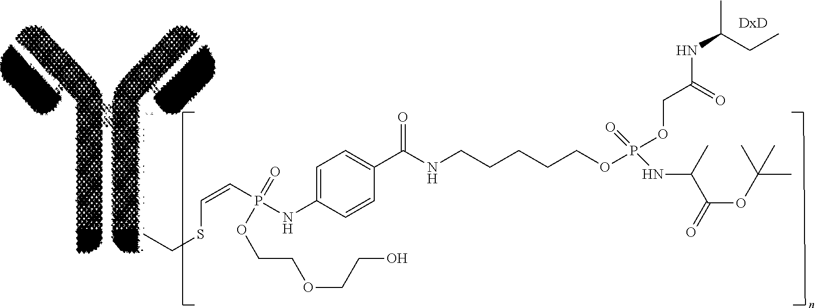 | see FIG. 33 | see FIG. 34 | DARav: 7.8 LC: calcd.: 24520 found: 24519 HC: calcd.: 52389 found: 52392 |
| Trastuzumab-O-P5(PEG2)-amidopentyl-Phosphoramidate-N-(L-alanine-tert.-butylester)-O-DxD | | | |
| 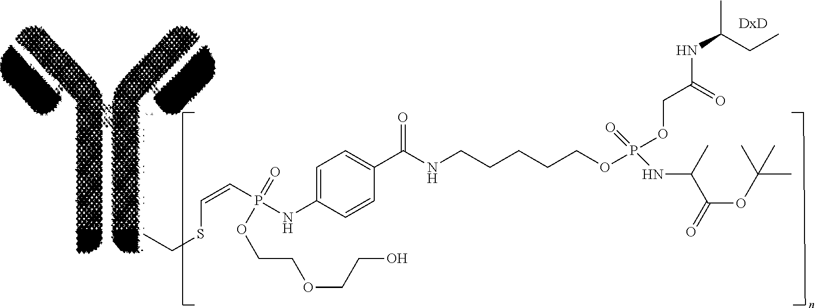 | see FIG. 35 | see FIG. 36 | DARav: 8.0 LC: calcd.: 24377 found: 24376 HC: calcd.: 52443 found: 52445 |
| Palivizumab-O-P5(PEG2)-amidopentyl-Phosphoramidate-N-(L-alanine-tert.-butylester)-O-DxD | | | |
| 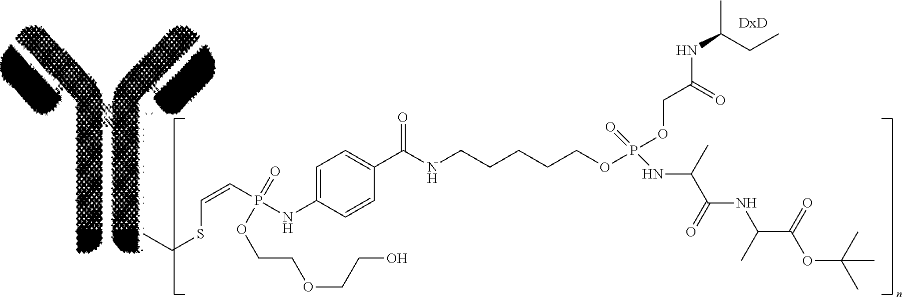 | see FIG. 37 | see FIG. 38 | DARav: 7.8 LC: calcd.: 24591 found: 24590 HC: calcd.: 52602 found: 52606 |
| Trastuzumab-O-P5(PEG2)-amidopentyl-Phosphoramidate-N-(L-alanine-L-alanine-tert.-butylester)-O-DxD | | | |
| 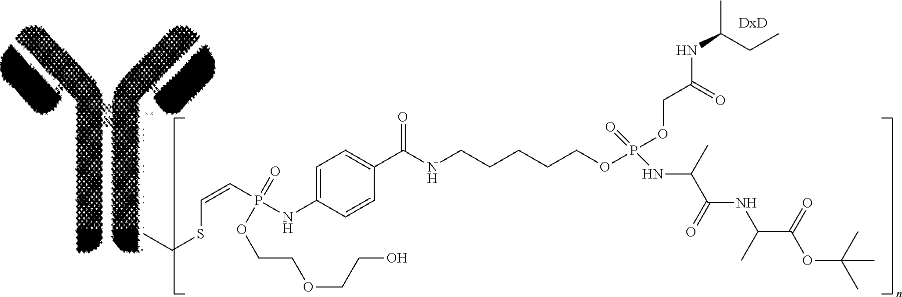 | see FIG. 39 | see FIG. 40 | DARav: 7.9 LC: calcd.: 24448 found: 24447 HC: calcd.: 52656 found: 52658 |
| Palivizumab-O-P5(PEG2)-amidopentyl-Phosphoramidate-N-(L-alanine-L-alanine-tert.-butylester)-O-DxD | | | |
| 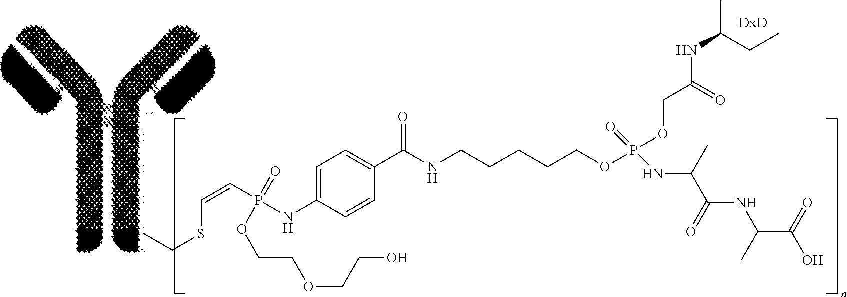 | see FIG. 41 | see FIG. 42 | DARav: 7.0 LC: calcd.: 24535 found: 24534 HC: calcd.: 52434, found: 52437, 51341, 50245 |
| Trastuzumab-O-P5(PEG2)-amidopentyl-Phosphoramidate-N-(L-alanine-L-alanine-COOH)-O-DxD | | | |
| 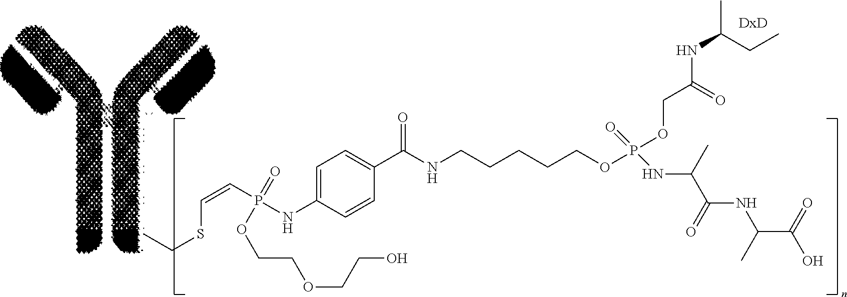 | see FIG. 43 | see FIG. 44 | LC: 7.8 calcd.: 24392 found: 24391 HC: calcd.: 52488 found: 52490, 51395, 50298 |
| Palivizumab-O-P5(PEG2)-amidopentyl-Phosphoramidate-N-(L-alanine-L-alanine-COOH)-O-DxD | | | |

| | | | MS analysis of the |
|---|---|---|---|
| | | | unconjugated mAb |
| | Analytical | Analytical | or fully conjugated |
| Antibody/ADC | SEC | HIC | DAR8 species, DAR |
| Brentuximab | see | see | LC: |
| | FIG. 250 | FIG. 251 | calcd.: 25518 |
| | | | found: 25514 |
| | | | HC: |
| | | | calcd.: 49028 |
| | | | found: 48877 |
| Datopotamab | see | see | LC: |
| | FIG. 252 | FIG. 253 | calcd.: 23402 |
| | | | found: 23397 |
| | | | HC: |
| | | | calcd.: 49116 |
| | | | found: 48981 |
| Brentuximab-O-P5(PEG24)- | see | see | DARav: 8.0 |
| amidopentyl-Phosphoramidate-N- | FIG. 189 | FIG. 190 | LC: |
| (L-alanine-iso-propylerster)-O- | | | calcd.: 27449 |
| SN38 | | | found: 27449 |
| | | | HC: |
| | | | calcd.: 54681 |
| | | | found: 54682 |
| Datopotamab-O-P5(PEG24)- | see | see | DARav: 8.0 |
| amidopentyl-Phosphoramidate-N- | FIG. 191 | FIG. 192 | LC: |
| (L-alanine-iso-propylerster)-O- | | | calcd.: 25332 |
| SN38 | | | found: 25332 |
| | | | HC: |
| | | | calcd.: 54786 |
| | | | found: 54787 |
| Brentuximab-O-P5(PEG24)- | see | see | DARav: 8.0 |
| amidopentyl-Phosphoramidate-N- | FIG. 193 | FIG. 194 | LC: |
| (glycine-tert.-butylester)-O-SN38 | | | calcd.: 27449 |
| | | | found: 27449 |
| | | | HC: |
| | | | calcd.: 54681 |
| | | | found: 54682 |
| Datopotamab-O-P5(PEG24)- | see | see | DARav: 8.0 |
| amidopentyl-Phosphoramidate-N- | FIG. 195 | FIG. 196 | LC: |
| (glycine-tert.-butylester)-O-SN38 | | | calcd.: 25332 |
| | | | found: 25332 |
| | | | HC |
| | | | calcd.: 54786 |
| | | | found: 54786 |
| Brentuximab-O-P5(PEG24)- | see | see | DARav: 8.0 |
| amidopentyl-Phosphoramidate-N- | FIG. 197 | FIG. 198 | LC: |
| (2,2-dimethylaminobutyric acid- | | | calcd.: 27477 |
| tert.-butylester)-O-SN38 | | | found: 27477 |
| | | | HC: |
| | | | calcd.: 54765 |
| | | | found: 54766 |
| Datopotamab-O-P5(PEG24)- | see | see | DARav: 8.0 |
| amidopentyl-Phosphoramidate-N- | FIG. 199 | FIG. 200 | LC: |
| (2,2-dimethylaminobutyric acid- | | | calcd.: 25360 |
| tert.-butylester)-O-SN38 | | | found: 25360 |
| | | | HC: |
| | | | calcd.: 54870 |
| | | | found: 54871 |
| Brentuximab-O-P5(PEG24)- | see | see | DARav: 8.0 |
| amidocyclohexyl- | FIG. 201 | FIG. 202 | LC |
| Phosphoramidate-N-(L-alanine-L- | | | calcd.: 27490 |
| alanine)-O-SN38 | | | found: 27490 |
| | | | HC: |
| | | | calcd.: 54804 |
| | | | found: 54805 |
| Datopotamab-O-P5(PEG24)- | see | see | DARav: 8.0 |
| amidocyclohexyl- | FIG. 203 | FIG. 204 | LC |
| Phosphoramidate-N-(L-alanine-L- | | | calcd.: 25373 |
| alanine)-O-SN38 | | | found: 25373 |
| | | | HC: |
| | | | calcd.: 54909 |
| | | | found: 54910 |
| Brentuximab-O-P5(PEG24)- | see | see | DARav: 8.0 |
| amidocyclohexyl- | FIG. 205 | FIG. 206 | LC: |
| Phosphoramidate-N-(L-alanine- | | | calcd.: 27475 |
| tert.-butylester)-O-SN38 | | | found: 27475 |
| | | | HC: |
| | | | calcd.: 54759 |
| | | | found: 54760 |
| Datopotamab-O-P5(PEG24)- | see | see | DARav: 8.0 |
| amidocyclohexyl- | FIG. 207 | FIG. 208 | LC: |
| Phosphoramidate-N-(L-alanine- | | | calcd.: 25358 |
| tert.-butylester)-O-SN38 | | | found: 25358 |
| | | | HC: |
| | | | calcd.: 54864 |
| | | | found: 54865 |
| Brentuximab-O-P5(PEG24)- | see | see | DARav: 8.0 |
| amidocyclohexyl- | FIG. 209 | FIG. 210 | LC: |
| Phosphoramidate-N-(L-alanine-iso- | | | calcd.: 27461 |
| propylester)-O-SN38 | | | found: 27461 |
| | | | HC: |
| | | | calcd.: 54717 |
| | | | found: 54718 |
| Datopotamab-O-P5(PEG24)- | see | see | DARav: 8.0 |
| amidocyclohexyl- | FIG. 211 | FIG. 212 | LC: |
| Phosphoramidate-N-(L-alanine-iso- | | | calcd.: 25344 |
| propylester)-O-SN38 | | | found: 25344 |
| | | | HC: |
| | | | calcd.: 54822 |
| | | | found: 54822 |
| Brentuximab-O-P5(PEG24)- | | | DARav: 6.2 |
| amidopentyl-Phosphoramidate-N- | | | LC: |
| (2-tert.-butyl-disulfide-ethyl)-O- | | | calcd.: 27483 |
| SN38 | | | found: 27483, 25514 |
| | | | HC: |
| | | | calcd.: 56228 |
| | | | found: 56228, 54259, |
| | | | 50320 |
| Datopotamab-O-P5(PEG24)- | | | DARav: 7.1 |
| amidopentyl-Phosphoramidate-N- | | | LC |
| (2-tert.-butyl-disulfide-ethyl)-O- | | | calcd.: 25366 |
| SN38 | | | found: 25366, 23398 |
| | | | HC: |
| | | | calcd.: 56332 |
| | | | found: 56333, 54363, |
| | | | 50425 |
| Brentuximab-O-P5(PEG24)- | | | DARav: 4.9 |
| amidopentyl-Phosphoramidate-N- | | | LC: |
| (2-tert.-butyl-disulfide-ethyl)-O-DXd | | | calcd.: 27584 |
| | | | found: 27584, 25514 |
| | | | HC: |
| | | | calcd.: 56531 |
| | | | found: 56531, 54461, |
| | | | 52391, 50320 |
| Datopotamab-O-P5(PEG24)- | | | DARav: 6.5 |
| amidopentyl-Phosphoramidate-N- | | | LC: |
| (2-tert.-butyl-disulfide-ethyl)-O-DXd | | | calcd.: 25467 |
| | | | found: 25467 |
| | | | HC: |
| | | | calcd.: 56636 |
| | | | found: 56636, 54565, |
| | | | 52494, 50425 |
| Brentuximab-5-Pentylacetamide- | see | see | DARav: 5.3 |
| Phosphoramidate-N-(L-alanine-L- | FIG. 213 | FIG. 214 | LC: |
| alanine)-O-DXd | | | calcd.: 26354 |
| | | | found: 26355 |
| | | | HC: |
| | | | calcd.: 51396 |
| | | | found: 51398, 50557, |
| | | | 49716, 48875 |
| Datopotamab-O-Pentylacetamide- | see | see | DARav: 4.4 |
| Phosphoramidate-N-(L-alanine-L- | FIG. 215 | FIG. 216 | LC: |
| alanine)-O-DXd | | | calcd.: 24237 |
| | | | found: 24238 |
| | | | HC: |
| | | | calcd.: 51501 |
| | | | found: 51503, 50662, |
| | | | 49822, 48981 |
| Brentuximab-6-(2,5- | see | see | DARav: 6.3 |
| dioxopyrrolidin-1-yl)-N-(5- | FIG. 217 | FIG. 218 | LC: |
| hydroxypentyl)hexanamido- | | | calcd.: 26507 |
| Phosphoramidate-N-(L-alanine-L- | | | found: 26508 |
| alanine)-O-DXd | | | HC: |
| | | | calcd.: 51855 |
| | | | found: 51857, 48970 |
| Datopotamab-6-(2,5- | see | see | DARav: 5.1 |
| dioxopyrrolidin-1-yl)-N-(5- | FIG. 219 | FIG. 220 | LC: |
| hydroxypentyl)hexanamido- | | | calcd.: 24390 |
| Phosphoramidate-N-(L-alanine-L- | | | found: 24391 |
| alanine)-O-DXd | | | HC: |
| | | | calcd.: 51960 |
| | | | found: 51962, 50968, |
| | | | 49975 |
| Brentuximab-O-P5(PEG24)- | see | see | DARav: 8.0 |
| amidopentyl-Phosphoramidate-N- | FIG. 221 | FIG. 222 | LC: |
| (L-alanine-L-alanine)-O-DHODH- | | | calcd.: 27522 |
| IN-16 | | | found: 27521 |
| | | | HC: |
| | | | calcd.: 56345 |
| | | | found: 56344 |
| Datopotamab-O-P5(PEG24)- | see | see | DARav: 8.0 |
| amidopentyl-Phosphoramidate-N- | FIG. 223 | FIG. 224 | LC: |
| (L-alanine-L-alanine)-O-DHODH- | | | calcd.: 25105 |
| IN-16 | | | found: 25403 |
| | | | HC: |
| | | | calcd.: 56449 |
| | | | found: 56449 |
| Brentuximab-O-P5(PEG24)- | see | see | DARav: 8.0 |
| amidopentyl-Phosphoramidate-N- | FIG. 225 | FIG. 226 | LC: |
| (L-alanine-L-alanine)-O-Roniciclib | | | calcd.: 27515 |
| | | | found: 27515 |
| | | | HC: |
| | | | calcd.: 54881 |
| | | | found: 54882 |
| Trastuzumab-O-P5(PEG24)- | see | see | DARav: 8.0 |
| amidopentyl-Phosphoramidate-N- | FIG. 227 | FIG. 228 | LC: |
| (L-alanine-L-alanine)-O-Roniciclib | | | calcd.: 25439 |
| | | | found: 25439 |
| | | | HC: |
| | | | calcd.: 55153 |
| | | | found: 55154 |
| Brentuximab-O-P5(PEG24)- | see | see | DARav: 8.0 |
| amidopentyl-Phosphoramidate-N- | FIG. 229 | FIG. 230 | LC: |
| (L-alanine-L-alanine)-O-Nampt-IN-1 | | | calcd.: 27561 |
| | | | found: 27561 |
| | | | HC |
| | | | calcd.: 55019 |
| | | | found: 55019 |
| Datopotamab-O-P5(PEG24)- | see | see | DARav: 8.0 |
| amidopentyl-Phosphoramidate-N- | FIG. 231 | FIG. 232 | LC: |
| (L-alanine-L-alanine)-O-Nampt-IN-1 | | | calcd.: 25444 |
| | | | found: 25444 |
| | | | HC: |
| | | | calcd.: 55123 |
| | | | found: 55125 |
| Brentuximab-O-P5(PEG24)- | see | see | DARav: 8.0 |
| amidopentyl-Phosphoramidate-N- | FIG. 233 | FIG. 234 | LC: |
| (L-alanine-L-alanine-tert.- | | | calcd.: 27995 |
| butylester)-O-Paclitaxel | | | found: 27996 |
| Trastuzumab-O-P5(PEG24)- | see | see | DARav: 8.0 |
| amidopentyl-Phosphoramidate-N- | FIG. 235 | FIG. 236 | LC: |
| (L-alanine-L-alanine-tert.- | | | calcd.: 25920 |
| butylester)-O-Paclitaxel | | | found: 25920 |
| Brentuximab-O-P5(PEG24)- | see | see | DARav: 7.0 |
| amidopentyl-Phosphoramidate-N- | FIG. 237 | FIG. 238 | LC: |
| (L-alanine-L-alanine-tert.- | | | calcd.: 27445 |
| butylester)-O-Triptolide | | | found: 27445 |
| | | | HC: |
| | | | calcd.: 54672 |
| | | | found: 54673, 52738 |
| Trastuzumab-O-P5(PEG24)- | see | see | DARav: 7.0 |
| amidopentyl-Phosphoramidate-N- | FIG. 239 | FIG. 240 | LC: |
| (L-alanine-L-alanine-tert.- | | | calcd.: 25369 |
| butylester)-O-Triptolide | | | found: 25371 |
| | | | HC: |
| | | | calcd.: 54945 |
| | | | found: 54944, 53010 |
| Brentuximab-O-P5(PEG24)- | see | see | DARav: 7.7 |
| amidopentyl-Phosphoramidate-N- | FIG. 241 | FIG. 242 | LC: |
| (L-alanine-L-alanine-)-O- | | | calcd.: 27480 |
| ON013100 | | | found: 27480, 25514 |
| | | | HC: |
| | | | calcd.: 54774 |
| | | | found: 54775 |
| Datopotamab-O-P5(PEG24)- | see | see | DARav: 8 |
| amidopentyl-Phosphoramidate-N- | FIG. 243 | FIG. 244 | LC: |
| (L-alanine-L-alanine-)-O- | | | calcd.: 25363 |
| ON013100 | | | found: 25363 |
| | | | HC: |
| | | | calcd.: 54879 |
| | | | found: 54880 |
| Brentuximab-O-P5(PEG24)- | | | DARav: 8.0 |
| amidopentyl-Phosphoramidate-N- | | | LC: |
| (L-alanine-iso-propylester)-O- | | | calcd.: 27451 |
| ON013100 | | | found: 27451 |
| | | | HC: |
| | | | calcd.: 56132 |
| | | | found: 56132 |
| Datopotamab-O-P5(PEG24)- | | | DARav: 8.0 |
| amidopentyl-Phosphoramidate-N- | | | LC: |
| (L-alanine-iso-propylester)-O- | | | calcd.: 25334 |
| ON013100 | | | found: 25334 |
| | | | HC: |
| | | | calcd.: 56236 |
| | | | found: 56237 |
| Brentuximab-O-P5(PEG24)- | see | see | DARav: 8.0 |
| amidopentyl-Phosphoramidate-N- | FIG. 245 | FIG. 246 | LC |
| (L-alanine-L-alanine)-O- | | | calcd.: 27450 |
| Ganetespib | | | found: 27450 |
| | | | HC: |
| | | | calcd.: 54684 |
| | | | found: 54685 |
| Datopotamab-O-P5(PEG24)- | see | see | DARav: 8.0 |
| amidopentyl-Phosphoramidate-N- | FIG. 247 | FIG. 248 | LC: |
| (L-alanine-L-alanine)-O- | | | calcd.: 25333 |
| Ganetespib | | | found: 25333 |
| | | | HC: |
| | | | calcd.: 54789 |
| | | | found: 54790 |
| Brentuximab-O-P5(PEG24)- | see | see | DARav: 8.0 |
| amidopentyl-Phosphoramidate-N- | FIG. 256 | FIG. 257 | LC: |
| (L-alanine-tert.-butylerster)-O- | | | calcd.: 27463 |
| SN38 | | | found: 27463 |
| | | | HC: |
| | | | calcd.: 54723 |
| | | | found: 54724 |
| Datopotamab-O-P5(PEG24)- | see | see | DARav: 8.0 |
| amidopentyl-Phosphoramidate-N- | FIG. 258 | FIG. 259 | LC: |
| (L-alanine-tert.-butylerster)-O- | | | calcd.: 25346 |
| SN38 | | | found: 25346 |
| | | | HC: |
| | | | calcd.: 54828 |
| | | | found: 54828 |
| Brentuximab-O-P5(PEG24)- | see | see | DARav: 8.0 |
| amidopentyl-Phosphoramidate-N- | FIG. 292 | FIG. 293 | LC: |
| (2-ethoxy-6-(3R,4S,5S,6S)- | | | calcd.: 27695 |
| (methoxycarbonyl)tetrahydro-2H- | | | found: 27695 |
| pyran-3,4,5-triyl triacetate)-O-SN38 | | | HC: |
| | | | calcd.: 55419 |
| | | | found: 55420 |
| Datopotamab-O-P5(PEG24)- | see | see | DARav: 8.0 |
| amidopentyl-Phosphoramidate-N- | FIG. 294 | FIG. 295 | LC: |
| (2-ethoxy-6-(3R,4S,5S,6S)- | | | calcd.: 25578 |
| (methoxycarbonyl)tetrahydro-2H- | | | found: 25578 |
| pyran-3,4,5-triyl triacetate)-O-SN38 | | | HC: |
| | | | calcd.: 5524 |
| | | | found: 55427 |
| Brentuximab-O-P5(PEG24)- | see | see | DARav: 8.0 |
| amidopentyl-Phosphoramidate-N- | FIG. 296 | FIG. 297 | LC: |
| (2-Acetoxy-ethyl)-O-DXD | | | calcd.: 27522 |
| | | | found: 27522 |
| | | | HC: |
| | | | calcd.: 54900 |
| | | | found: 54901 |
| Datopotamab-O-P5(PEG24)- | see | see | DARav: 8.0 |
| amidopentyl-Phosphoramidate-N- | FIG. 298 | FIG. 299 | LC: |
| (2-Acetoxy-ethyl)-O-DXD | | | calcd.: 25405 |
| | | | found: 25405 |
| | | | HC: |
| | | | calcd.: 55005 |
| | | | found: 55006 |
| Brentuximab-O-P5(PEG24)- | see | see | DARav: 8.0 |
| amidopentyl-Phosphoramidate-N- | FIG. 300 | FIG. 301 | LC: |
| (2-Acetamido-ethyl)-O-SN38 | | | calcd.: 27420 |
| | | | found: 27421 |
| | | | HC: |
| | | | calcd.: 54594 |
| | | | found: 54595 |
| Datopotamab-O-P5(PEG24)- | see | see | DARav: 8.0 |
| amidopentyl-Phosphoramidate-N- | FIG. 302 | FIG. 303 | LC |
| (2-Acetamido-ethyl)-O-SN38 | | | calcd.: 25303 |
| | | | found: 25303 |
| | | | HC: |
| | | | calcd.: 54699 |
| | | | found: 54700 |
| Brentuximab-O-P5-(PEG24)- | see | see | DARav: 8.0 |
| amidopentyl-Phosphoramidate-O- | FIG. 304 | FIG. 305 | LC: |
| (lactic acid-iso-propylester)-O- | | | calcd.: 27449 |
| SN38 | | | found: 27449 |
| | | | HC: |
| | | | calcd.: 54681 |
| | | | found: 54682 |
| Datopotamab-O-P5-(PEG24)- | see | see | DARav: 8.0 |
| amidopentyl-Phosphoramidate-O- | FIG. 306 | FIG. 307 | LC: |
| (lactic acid-iso-propylester)-O- | | | calcd.: 25332 |
| SN38 | | | found: 25332 |
| | | | HC: |
| | | | calcd.: 54786 |
| | | | found: 54787 |
| Brentuximab-O-P5-(PEG24)- | see | see | DARav: 8.0 |
| amidopentyl-Phosphoramidate-O- | FIG. 308 | FIG. 309 | LC: |
| (glycolic acid-iso-propylester)-N- | | | calcd.: 27479 |
| Exatecan | | | found: 27479 |
| | | | HC: |
| | | | calcd.: 54771 |
| | | | found: 54772 |
| Datopotamab-O-P5-(PEG24)- | see | see | DARav: 8.0 |
| amidopentyl-Phosphoramidate-O- | FIG. 310 | FIG. 311 | LC: |
| (glycolic acid-iso-propylester)-N- | | | calcd.: 25362 |
| Exatecan | | | found: 25362 |
| | | | HC: |
| | | | calcd.: 54876 |
| | | | found: 54877 |
| Trastuzumab-O-P5(PEG24)- | see | see | DARav: 8.0 |
| amidopentyl-Phosphoramidate-N- | FIG. 312 | FIG. 313 | LC: |
| (L-alanine-L-alanine-)-O- | | | calcd.: 25405 |
| ON013100 | | | found: 25405 |
| | | | HC: |
| | | | calcd.: 55047 |
| | | | found: 55048 |
| Trastuzumab-O-P5(PEG24)- | see | see | DARav: 8.0 |
| amidopentyl-Phosphoramidate-N- | FIG. 318 | FIG. 319 | LC: |
| (L-alanine-iso-propylester)-O- | | | calcd.: 25376 |
| ON013100 | | | found: 25375 |
| | | | HC: |
| | | | calcd.: 56404 |
| | | | found: 56405 |
| Trastuzumab-O-P5(PEG24)- | see | see | DARav: 8.0 |
| amidopentyl-Phosphoramidate-N- | FIG. 260 | FIG. 261 | LC: |
| (L-alanine-L-alanine)-O- | | | calcd.: 25375 |
| Ganetespib | | | found: 25375 |
| | | | HC: |
| | | | calcd.: 54957 |
| | | | found: 54958 |
| Trastuzumab-O-P5(PEG24)- | | | DARav: 8.0 |
| amidopentyl-Phosphoramidate-N- | | | LC: |
| (L-alanine-iso-propylester)-O- | | | calcd.: 25346 |
| Ganetespib | | | found: 25345 |
| | | | HC: |
| | | | calcd.: 56314 |
| | | | found: 56315 |
| Palivizumab-O-P5-(PEG24)- | see | see | DARav: 8.0 |
| amidopentyl-Phosphoramidate-N- | FIG. 262 | FIG. 263 | LC: |
| (L-alanine-L-alanine)-O-BAY- | | | calcd.: 25387 |
| 2402234 | | | found: 25387 |
| | | | HC: |
| | | | calcd.: 55478 |
| | | | found: 55480 |
| Datopotamab-O-P5-(PEG24)- | see | see | DARav: 8.0 |
| amidopentyl-Phosphoramidate-N- | FIG. 264 | FIG. 265 | LC: |
| (L-alanine-L-alanine)-O-BAY- | | | calcd.: 25489 |
| 2402234 | | | found: 25489 |
| | | | HC: |
| | | | calcd.: 55256 |
| | | | found: 55258 |
| Brentuximab-O-P5(PEG24)- | see | see | DARav: 6.2 |
| amidopentyl-Phosphoramidate-N- | FIG. 284 | FIG. 285 | LC: |
| (2-tert.-butyl-disulfide-ethyl)-O- | | | calcd.: 27483 |
| SN38 | | | found: 27483, 25514 |
| | | | HC: |
| | | | calcd.: 56228 |
| | | | found: 56228, 54259, |
| | | | 50320 |
| Datopotamab-O-P5(PEG24)- | see | see | DARav: 7.1 |
| amidopentyl-Phosphoramidate-N- | FIG. 286 | FIG. 287 | LC: |
| (2-tert.-butyl-disulfide-ethyl)-O- | | | calcd.: 25366 |
| SN38 | | | found: 25366, 23398 |
| | | | HC: |
| | | | calcd.: 56332 |
| | | | found: 56333, 54363, |
| | | | 50425 |
| Brentuximab-O-P5(PEG24)- | see | see | DARav: 4.9 |
| amidopentyl-Phosphoramidate-N- | FIG. 288 | FIG. 289 | LC: |
| (2-tert.-butyl-disulfide-ethyl)-O- | | | calcd.: 27584 |
| DXD | | | found: 27584, 25514 |
| | | | HC: |
| | | | calcd.: 56531 |
| | | | found: 56531, 54461, |
| | | | 50320 |
| O-P5(PEG24)-amidopentyl- | see | see | DARav: 6.6 |
| Phosphoramidate-N-(2-tert.-butyl- | FIG. 290 | FIG. 291 | LC: |
| disulfide-ethyl)-O-DXD | | | calcd.: 25467 |
| | | | found: 25467, 23398 |
| | | | HC: |
| | | | calcd.: 56635 |
| | | | found: 56636, 54566, |
| | | | 50425 |
| Brentuximab-O-P5(PEG24)- | see | see | DARav: 8.0 |
| amidopentyl-Phosphoramidate-N- | FIG. 314 | FIG. 315 | LC: |
| (L-alanine-iso-propylester)-O- | | | calcd.: 27451 |
| ON013100 | | | found: 27451 |
| | | | HC: |
| | | | calcd.: 56132 |
| | | | found: 56132 |
| Datopotamab-O-P5(PEG24)- | see | see | DARav: 8.0 |
| amidopentyl-Phosphoramidate-N- | FIG. 316 | FIG. 317 | LC: |
| (L-alanine-iso-propylester)-O- | | | calcd.: 25334 |
| ON013100 | | | found: 25334 |
| | | | HC: |
| | | | calcd.: 56236 |
| | | | found: 56237 |
| Brentuximab-O-P5(PEG24)- | see | see | DARav: 8.0 |
| amidopentyl-Phosphoramidate-N- | FIG. 320 | FIG. 321 | LC: |
| (L-alanine-isopropylester)-O- | | | calcd.: 27421 |
| Ganetespib | | | found: 27421 |
| | | | HC: |
| | | | calcd.: 56042 |
| | | | found: 56042 |
| Datopotamab-O-P5(PEG24)- | see | see | DARav: 8.0 |
| amidopentyl-Phosphoramidate-N- | FIG. 322 | FIG. 323 | LC: |
| (L-alanine-isopropylester)-O- | | | calcd.: 25304 |
| Ganetespib | | | found: 25304 |
| | | | HC: |
| | | | calcd.: 56146 |
| | | | found: 56147 |

FIG. 21 shows an analytical SEC chromatogram of Trastuzumab. SEC means size exclusion chromatography.

FIG. 22 shows an analytical HIC chromatogram of Trastuzumab. HIC means hydrophobic interaction chromatography.

FIG. 23 shows an analytical SEC chromatogram of Sacituzumab.

FIG. 24 shows an analytical HIC chromatogram of Sacituzumab.

FIG. 25 shows an analytical SEC chromatogram of Palivizumab.

FIG. 26 shows an analytical HIC chromatogram of Palivizumab.

FIG. 27 shows an analytical SEC chromatogram of Sacituzumab-O—P5(PEG2)-amidopentyl-Phosphoramidate-N-(L-alanine-tert.-butylester)-O—SN38.

FIG. 28 shows an analytical HIC chromatogram of Sacituzumab-O—P5(PEG2)-amidopentyl-Phosphoramidate-N-(L-alanine-tert.-butylester)-O—SN38.

FIG. 29 shows an analytical SEC chromatogram of Sacituzumab-O—P5(PEG12)-amidopentyl-Phosphoramidate-N-(L-alanine-tert.-butylester)-O—SN38.

FIG. 30 shows an analytical HIC chromatogram of Sacituzumab-O—P5(PEG12)-amidopentyl-Phosphoramidate-N-(L-alanine-tert.-butylester)-O—SN38.

FIG. 31 shows an analytical SEC chromatogram of Palivizumab-O—P5(PEG12)-amidopentyl-Phosphoramidate-N-(L-alanine-tert.-butylester)-O—SN38.

FIG. 32 shows an analytical HIC chromatogram of Palivizumab-O—P5(PEG12)-amidopentyl-Phosphoramidate-N-(L-alanine-tert.-butylester)-O—SN38.

FIG. 33 shows an analytical SEC chromatogram of Trastuzumab-O—P5(PEG2)-amidopentyl-Phosphoramidate-N-(L-alanine-tert.-butylester)-O-DxD.

FIG. 34 shows an analytical HIC chromatogram of Trastuzumab-O—P5(PEG2)-amidopentyl-Phosphoramidate-N-(L-alanine-tert.-butylester)-O-DxD.

FIG. 35 shows an analytical SEC chromatogram of Palivizumab-O—P5(PEG2)-amidopentyl-Phosphoramidate-N-(L-alanine-tert.-butylester)-O-DxD.

FIG. 36 shows an analytical HIC chromatogram of Palivizumab-O—P5(PEG2)-amidopentyl-Phosphoramidate-N-(L-alanine-tert.-butylester)-O-DxD.

FIG. 37 shows an analytical SEC chromatogram of Trastuzumab-O—P5(PEG2)-amidopentyl-Phosphoramidate-N-(L-alanine-L-alanine-tert.-butylester)-O-DxD.

FIG. 38 shows an analytical HIC chromatogram of Trastuzumab-O—P5(PEG2)-amidopentyl-Phosphoramidate-N-(L-alanine-L-alanine-tert.-butylester)-O-DxD.

FIG. 39 shows an analytical SEC chromatogram of Palivizumab-O—P5(PEG2)-amidopentyl-Phosphoramidate-N-(L-alanine-L-alanine-tert.-butylester)-O-DxD.

FIG. 40 shows an analytical HIC chromatogram of Palivizumab-O—P5(PEG2)-amidopentyl-Phosphoramidate-N-(L-alanine-L-alanine-tert.-butylester)-O-DxD.

FIG. 41 shows an analytical SEC chromatogram of Trastuzumab-O—P5(PEG2)-amidopentyl-Phosphoramidate-N-(L-alanine-L-alanine —COOH)—O-DxD.

FIG. 42 shows an analytical HIC chromatogram of Trastuzumab-O—P5(PEG2)-amidopentyl-Phosphoramidate-N-(L-alanine-L-alanine-COOH)—O-DxD.

FIG. 43 shows an analytical SEC chromatogram of Palivizumab-O—P5(PEG2)-amidopentyl-Phosphoramidate-N-(L-alanine-L-alanine-COOH)—O-DxD.

FIG. 44 shows an analytical HIC chromatogram of Palivizumab-O—P5(PEG2)-amidopentyl-Phosphoramidate-N-(L-alanine-L-alanine-COOH)—O-DxD.

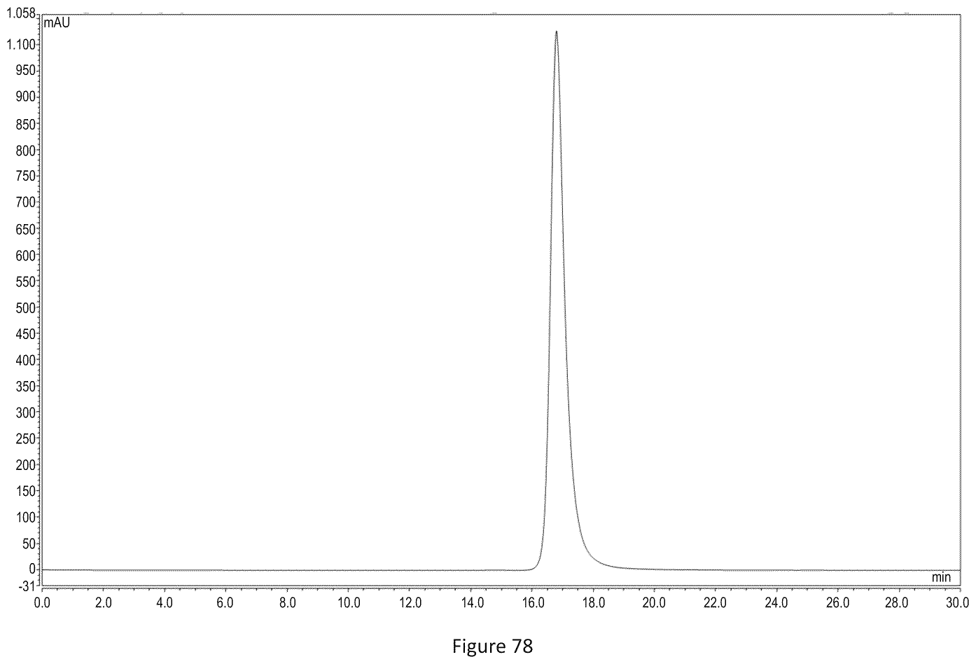

FIG. 78 shows an analytical SEC chromatogram of Trastuzumab-O—P5(PEG24)-amidopentyl-Phosphoramidate-N-(L-alanine-tert.-butylester)-O-OTS-964.

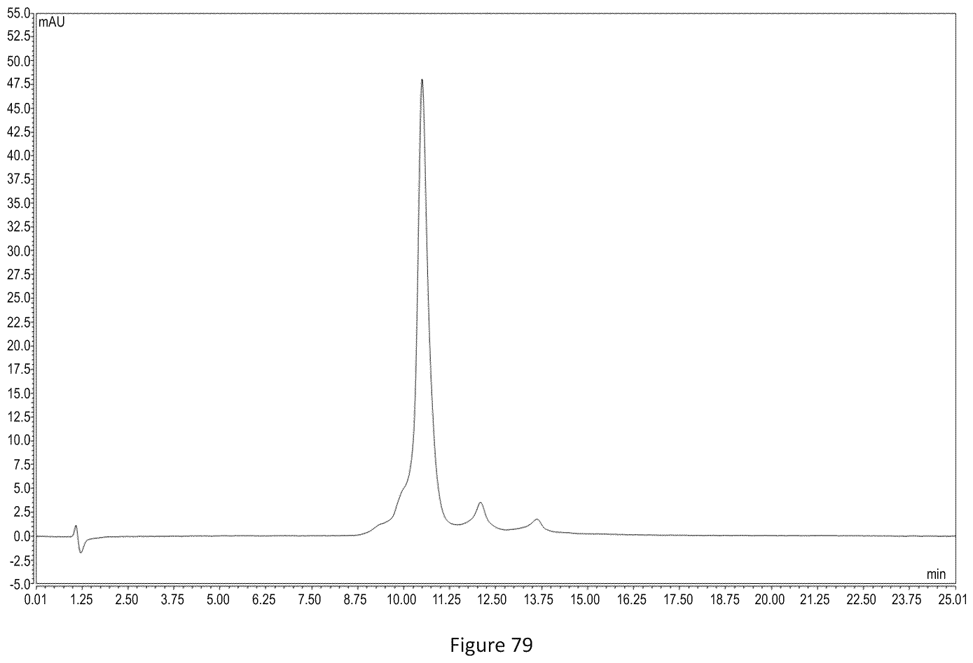

FIG. 79 shows an analytical HIC chromatogram of Trastuzumab-O—P5(PEG24)-amidopentyl-Phosphoramidate-N-(L-alanine-tert.-butylester)-O-OTS-964.

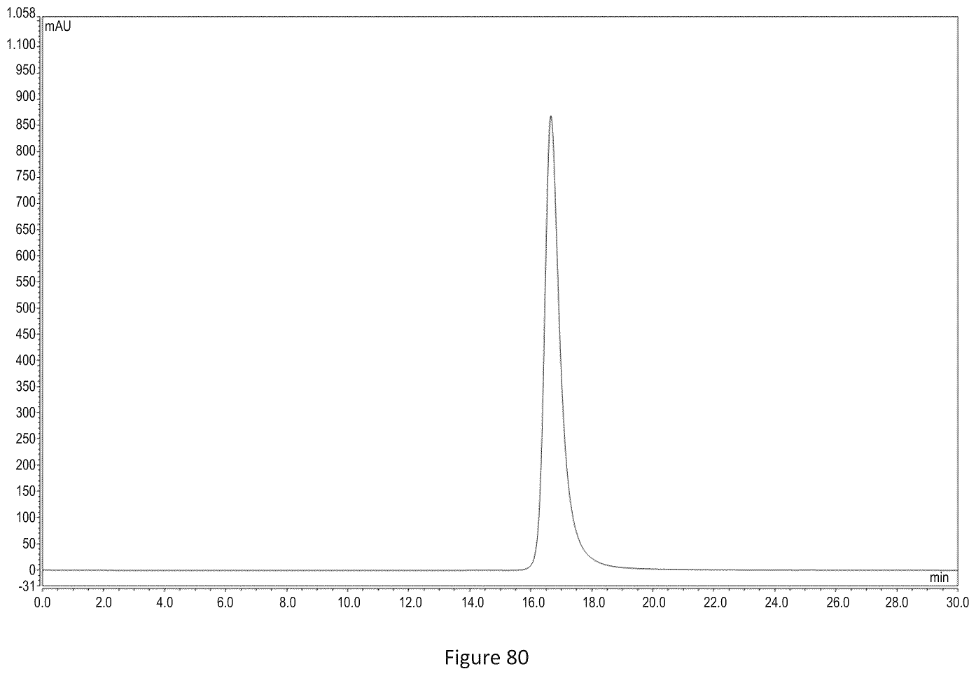

FIG. 80 shows an analytical SEC chromatogram of Palivizumab-O—P5(PEG24)-amidopentyl-Phosphoramidate-N-(L-alanine-tert.-butylester)-O-OTS-964.

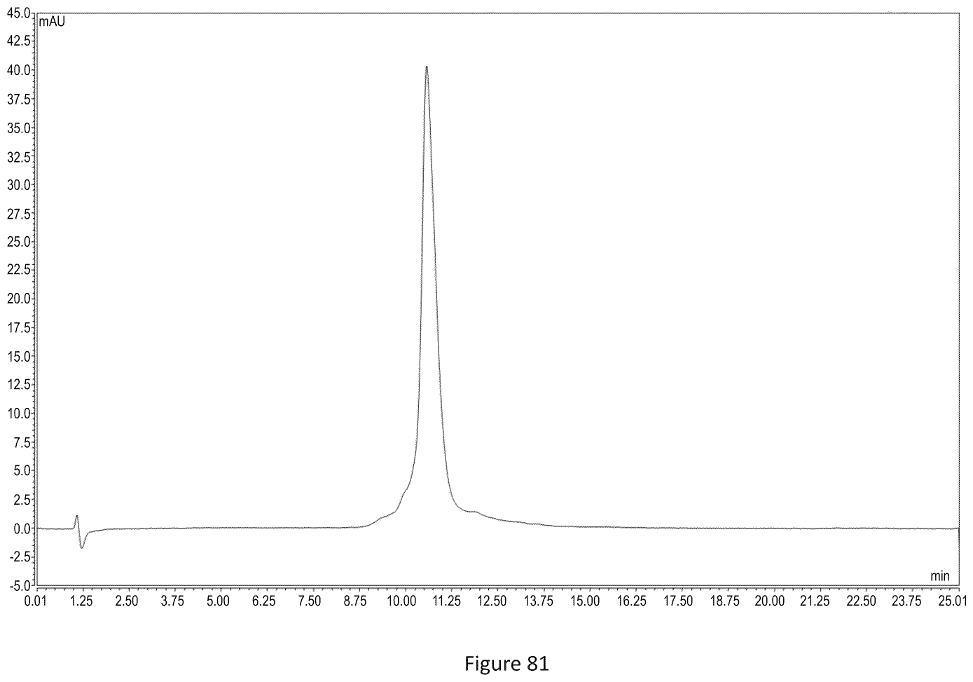

FIG. 81 shows an analytical HIC chromatogram of Palivizumab-O—P5(PEG24)-amidopentyl-Phosphoramidate-N-(L-alanine-tert.-butylester)-O-OTS-964.

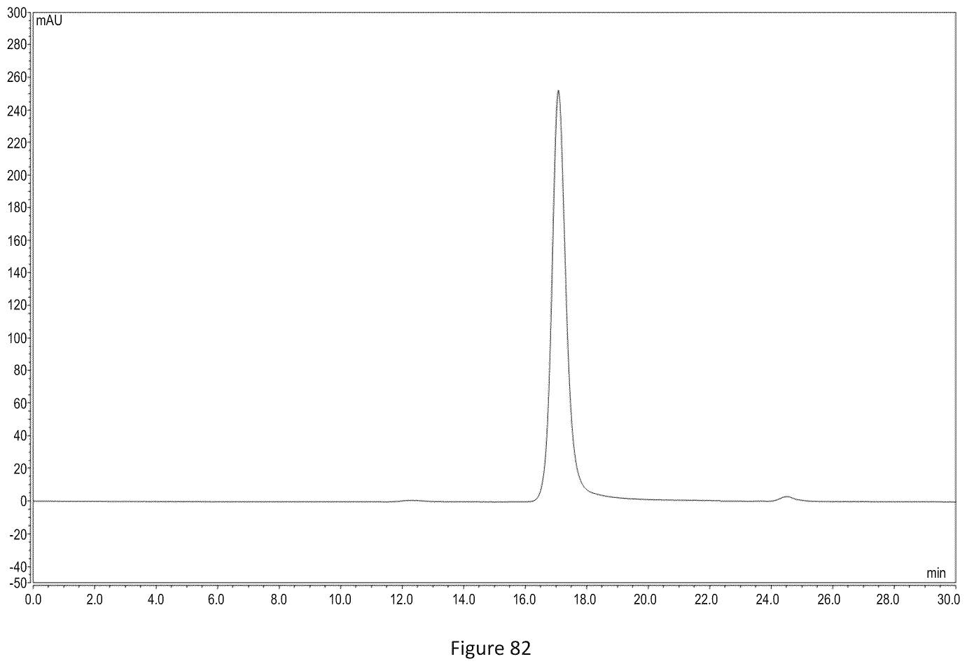

FIG. 82 shows an analytical SEC chromatogram of Trastuzumab-O—P5(PEG24)-amidopentyl-Phosphoramidate-N-(L-alanine-tert.-butylester)-O-Ganetespib.

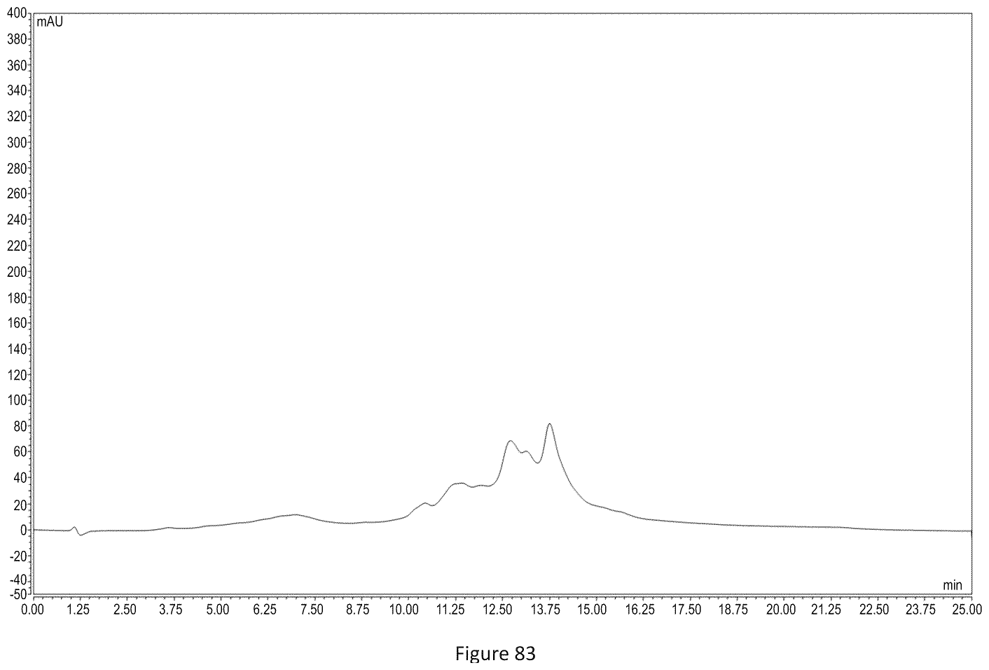

FIG. 83 shows an analytical HIC chromatogram of Trastuzumab-O—P5(PEG24)-amidopentyl-Phosphoramidate-N-(L-alanine-tert.-butylester)-O-Ganetespib.

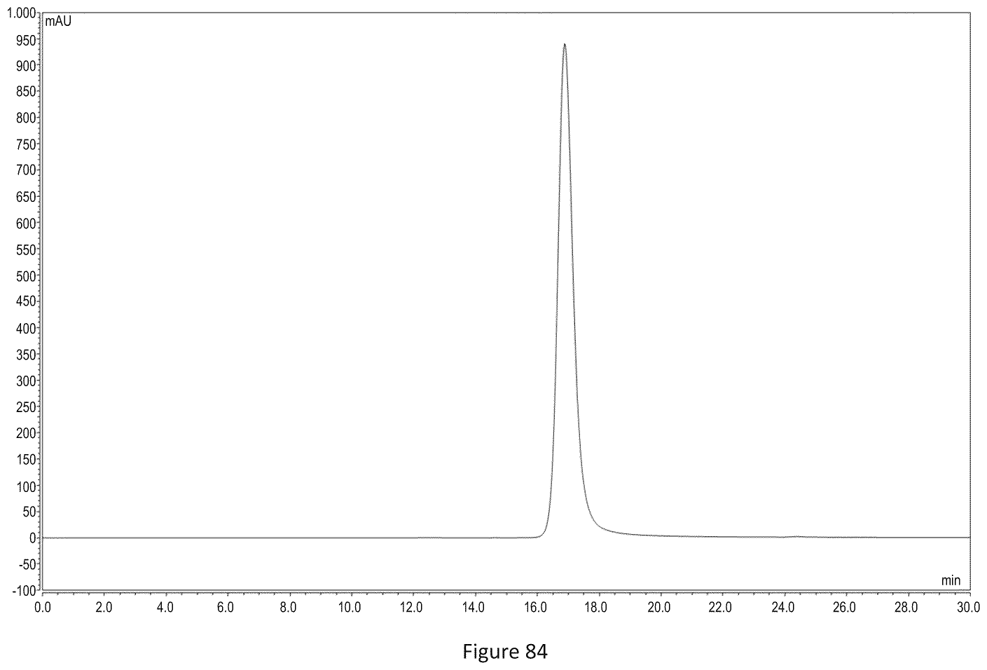

FIG. 84 shows an analytical SEC chromatogram of Palivizumab-O—P5(PEG24)-amidopentyl-Phosphoramidate-N-(L-alanine-tert.-butylester)-O-Ganetespib.

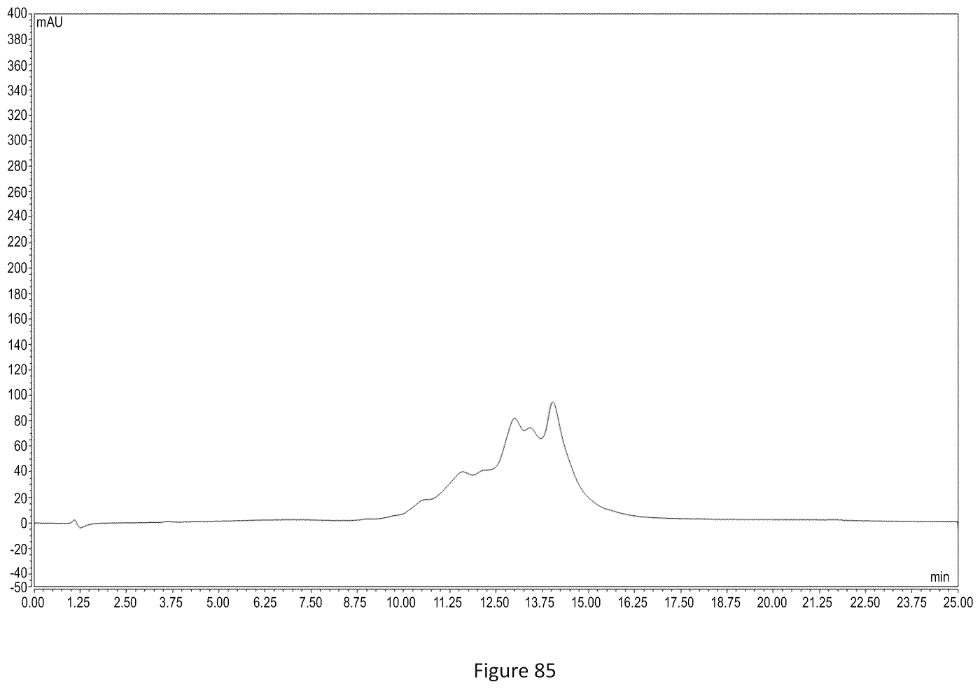

FIG. 85 shows an analytical HIC chromatogram of Palivizumab-O—P5(PEG24)-amidopentyl-Phosphoramidate-N-(L-alanine-tert.-butylester)-O-Ganetespib.

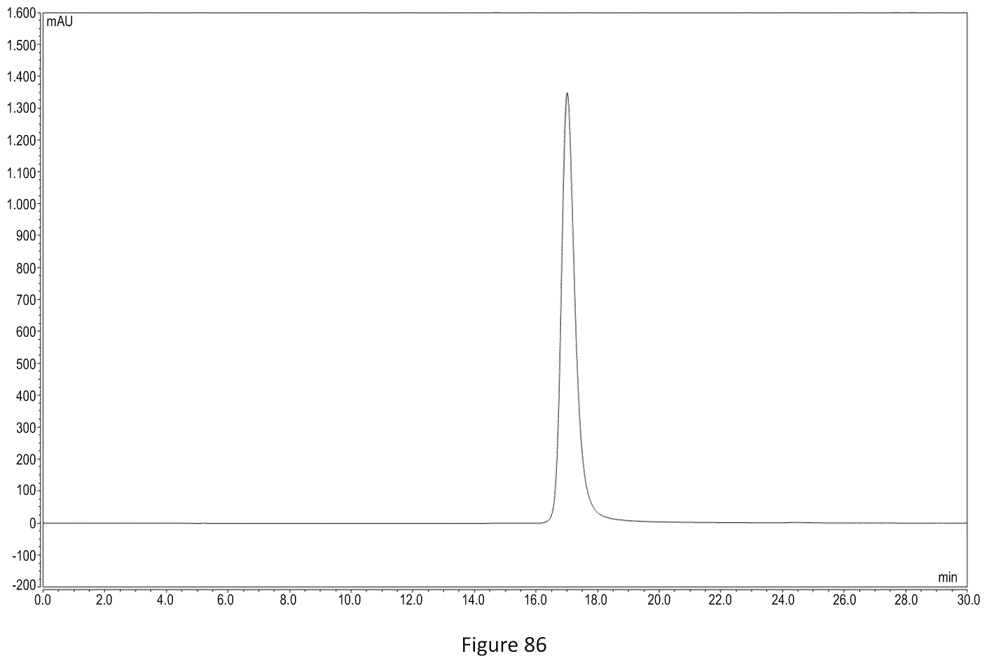

FIG. 86 shows an analytical SEC chromatogram of Trastuzumab-O—P5(PEG24)-amidopentyl-Phosphoramidate-N-(L-alanine-tert.-butylester)-O-Birabresib.

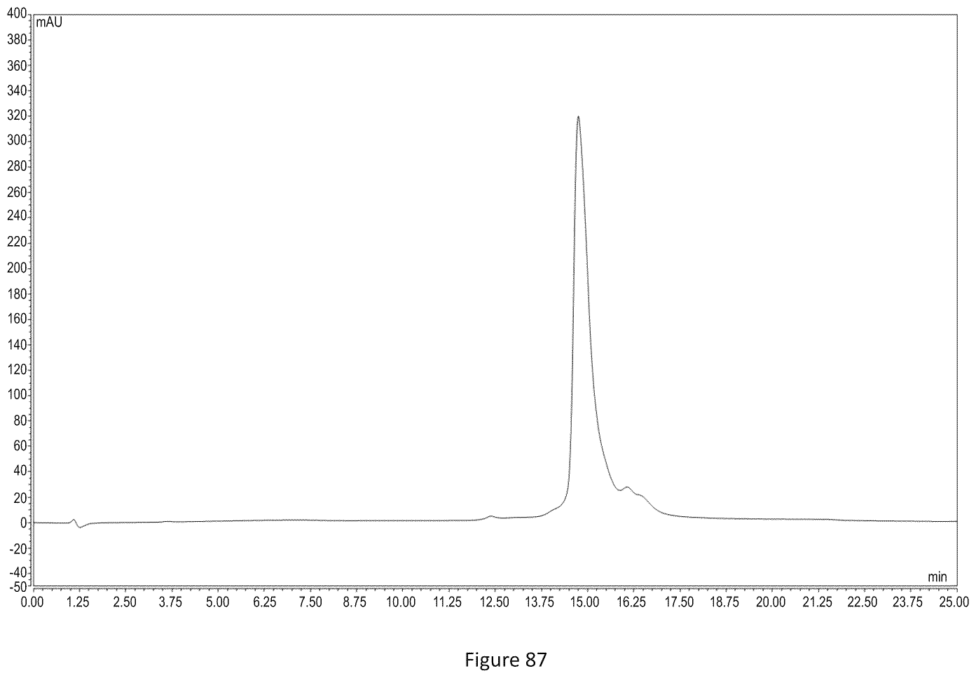

FIG. 87 shows an analytical HIC chromatogram of Trastuzumab-O—P5(PEG24)-amidopentyl-Phosphoramidate-N-(L-alanine-tert.-butylester)-O-Birabresib.

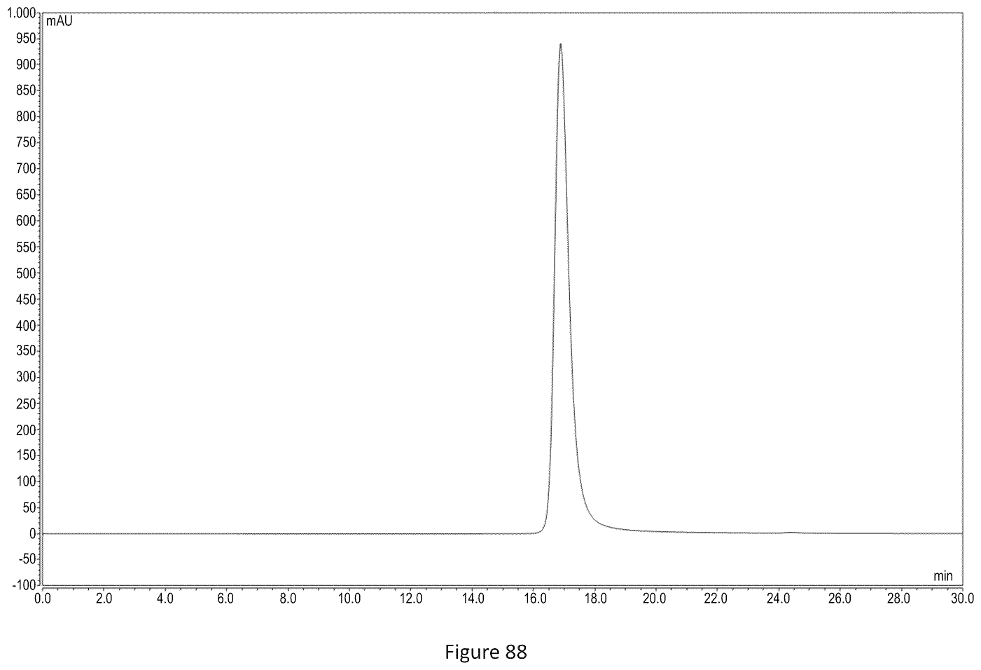

FIG. 88 shows an analytical SEC chromatogram of Palivizumab-O—P5(PEG24)-amidopentyl-Phosphoramidate-N-(L-alanine-tert.-butylester)-O-Birabresib.

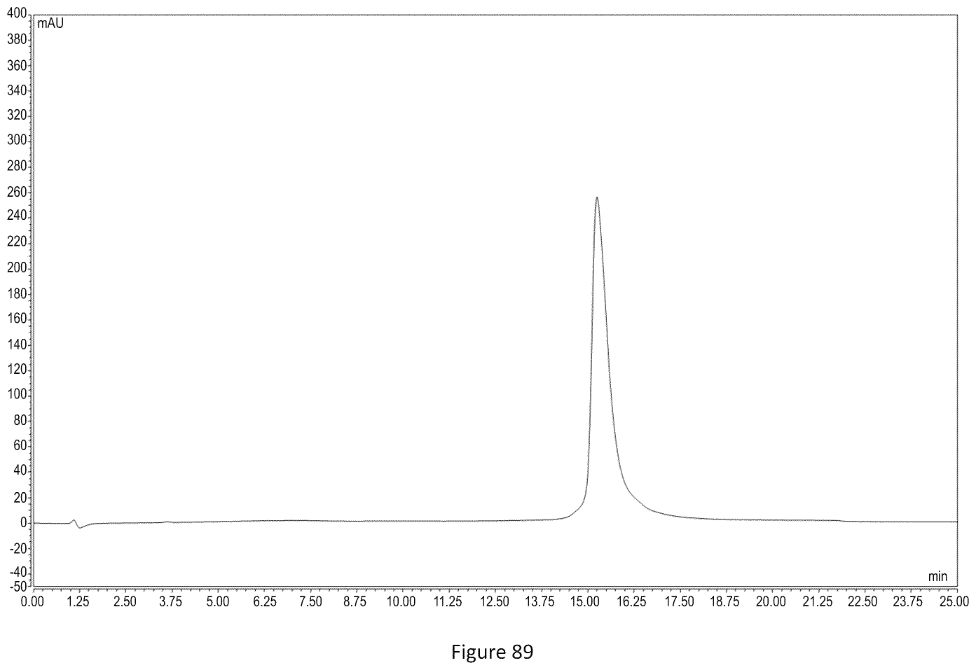

FIG. 89 shows an analytical HIC chromatogram of Palivizumab-O—P5(PEG24)-amidopentyl-Phosphoramidate-N-(L-alanine-tert.-butylester)-O-Birabresib.

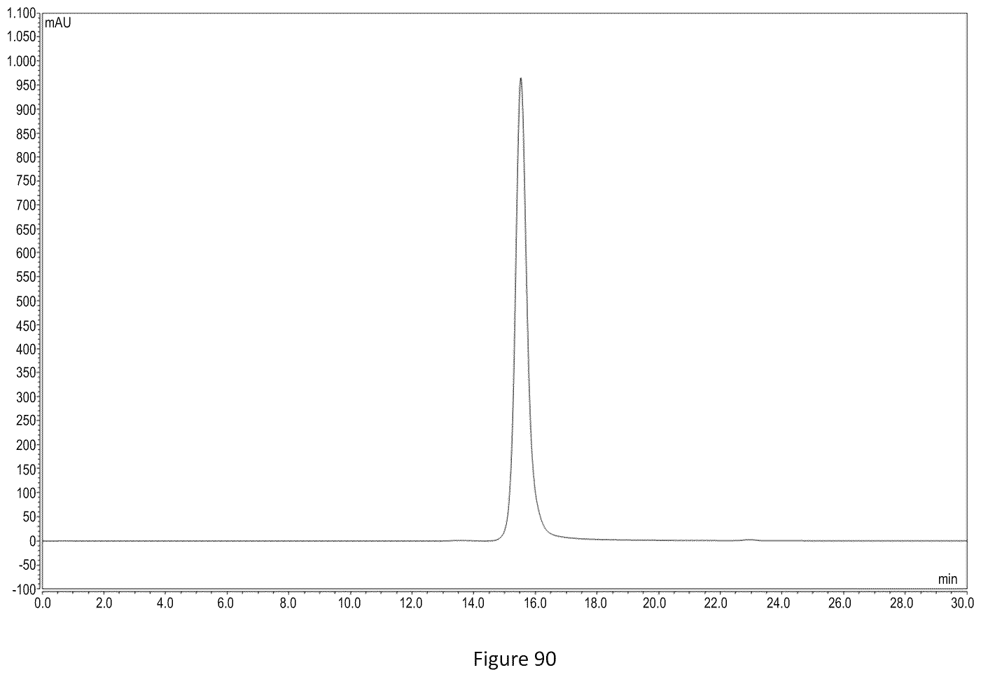

FIG. 90 shows an analytical SEC chromatogram of Trastuzumab-O—P5(PEG24)-amidopentyl-Phosphoramidate-N-(L-alanine-L-alanine)-O—SNX-2112.

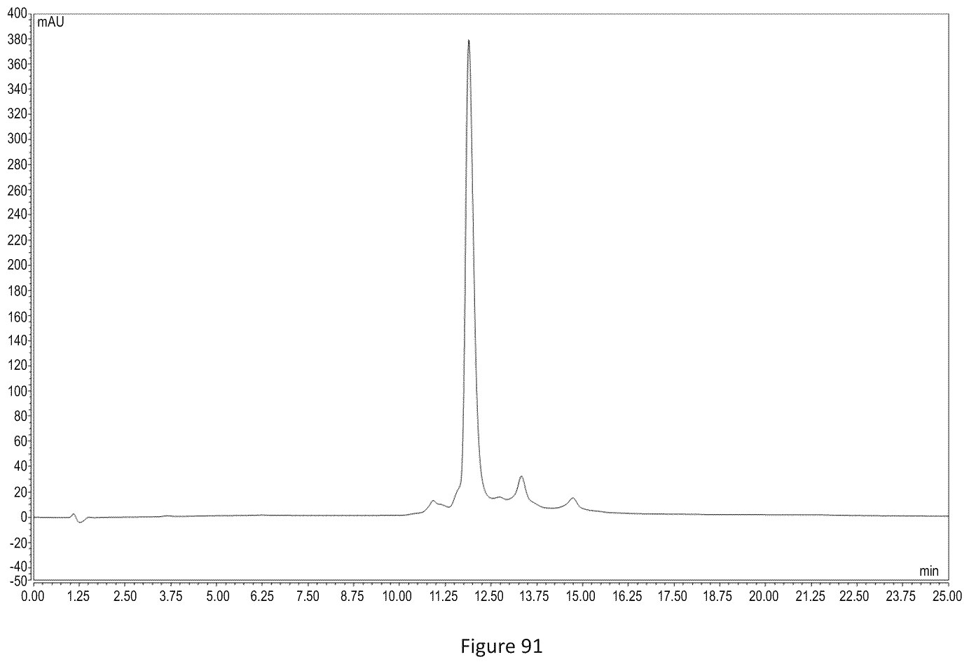

FIG. 91 shows an analytical HIC chromatogram of Trastuzumab-O—P5(PEG24)-amidopentyl-Phosphoramidate-N-(L-alanine-L-alanine)-O—SNX-2112.

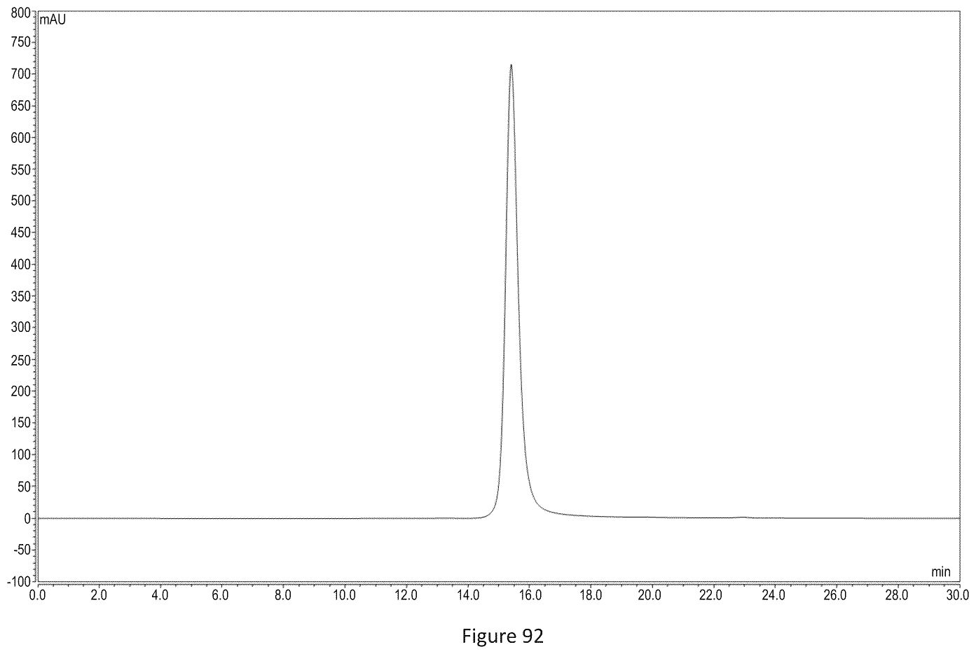

FIG. 92 shows an analytical SEC chromatogram of Palivizumab-O—P5(PEG24)-amidopentyl-Phosphoramidate-N-(L-alanine-L-alanine)-O—SNX-2112.

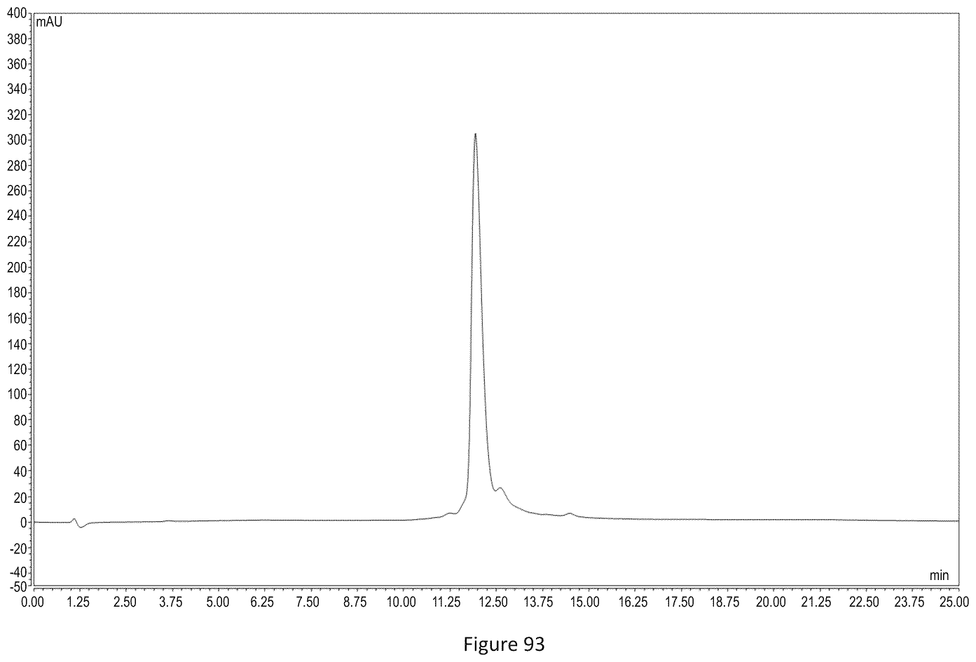

FIG. 93 shows an analytical HIC chromatogram of Palivizumab-O—P5(PEG24)-amidopentyl-Phosphoramidate-N-(L-alanine-L-alanine)-O—SNX-2112.

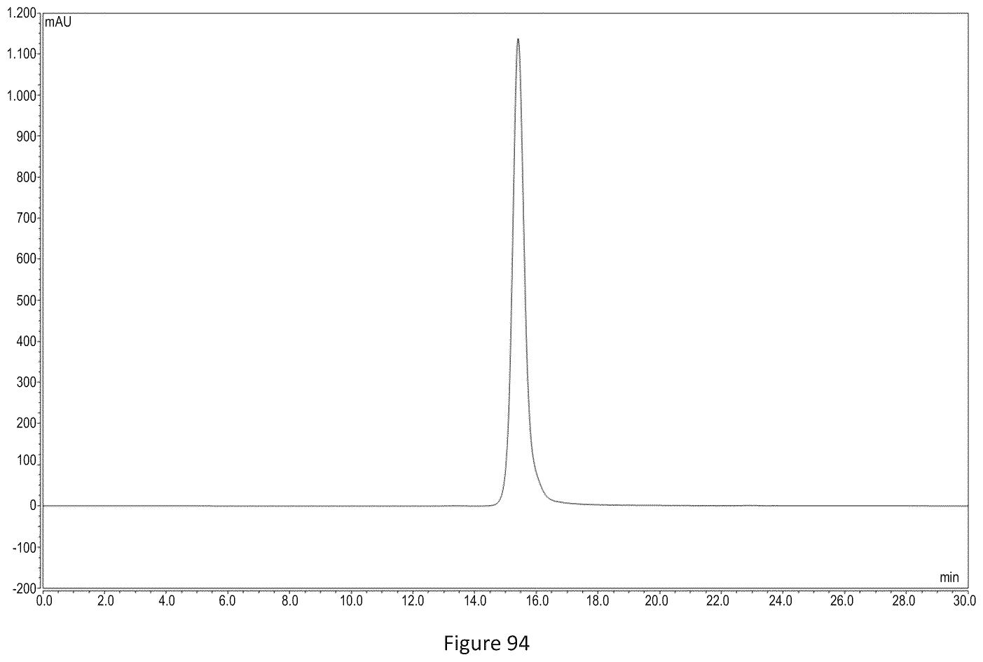

FIG. 94 shows an analytical SEC chromatogram of Trastuzumab-O—P5(PEG24)-amidopentyl-Phosphoramidate-N-(L-alanine-L-alanine)-O-Gemcitabine.

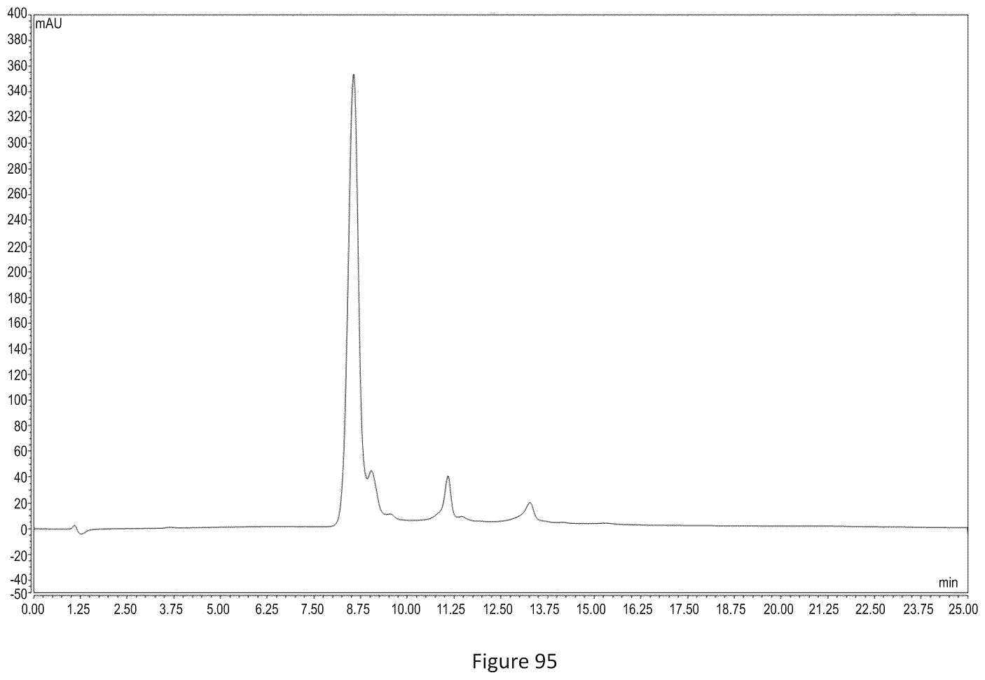

FIG. 95 shows an analytical HIC chromatogram of Trastuzumab-O—P5(PEG24)-amidopentyl-Phosphoramidate-N-(L-alanine-L-alanine)-O-Gemcitabine.

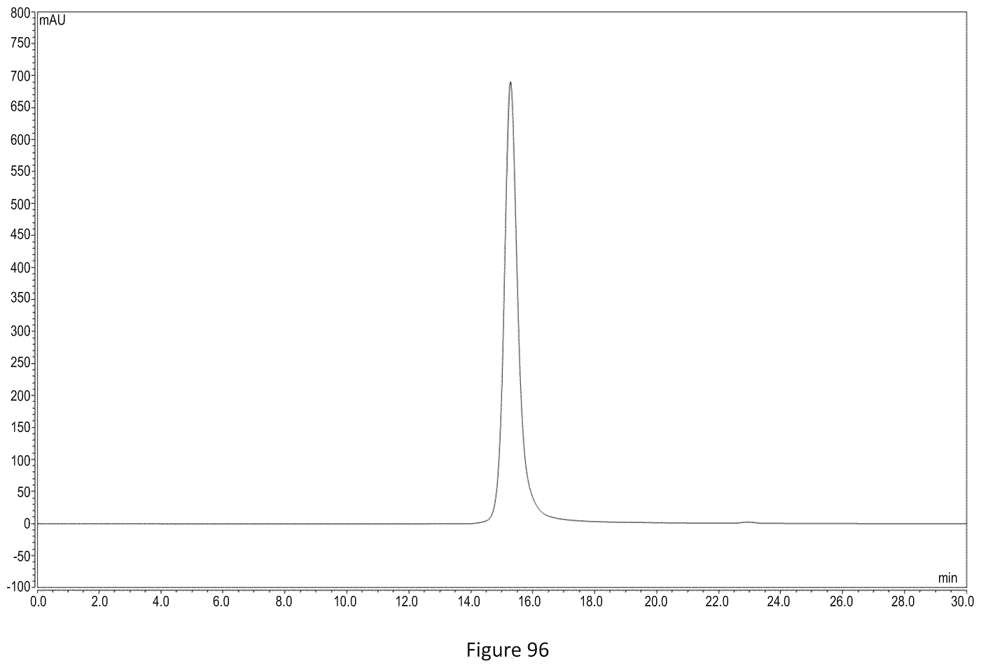

FIG. 96 shows an analytical SEC chromatogram of Palivizumab-O—P5(PEG24)-amidopentyl-Phosphoramidate-N-(L-alanine-L-alanine)-O-Gemcitabine.

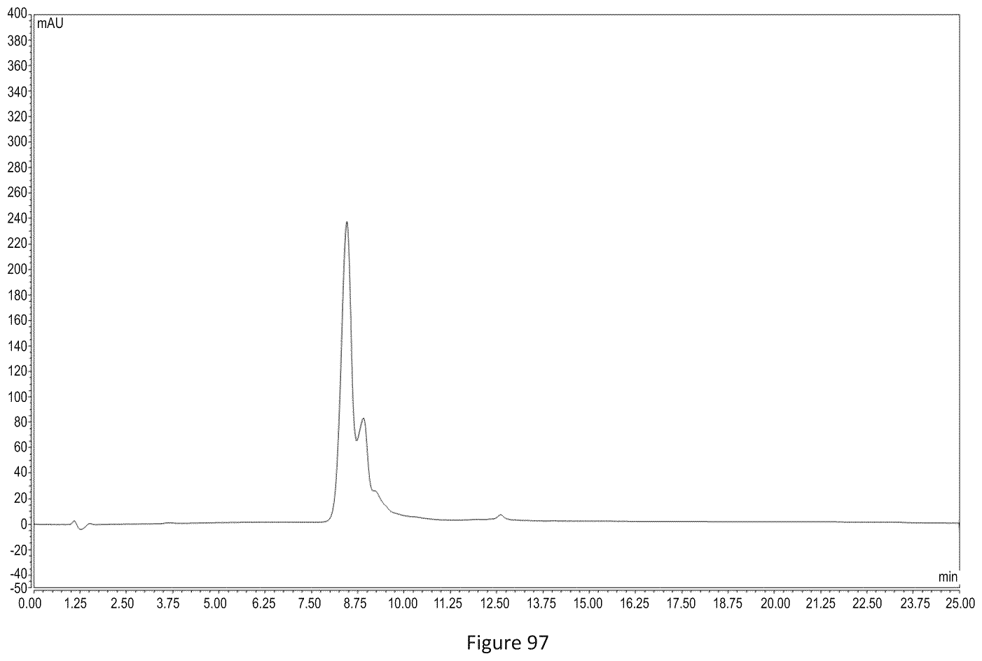

FIG. 97 shows an analytical HIC chromatogram of Palivizumab-O—P5(PEG24)-amidopentyl-Phosphoramidate-N-(L-alanine-L-alanine)-O-Gemcitabine.

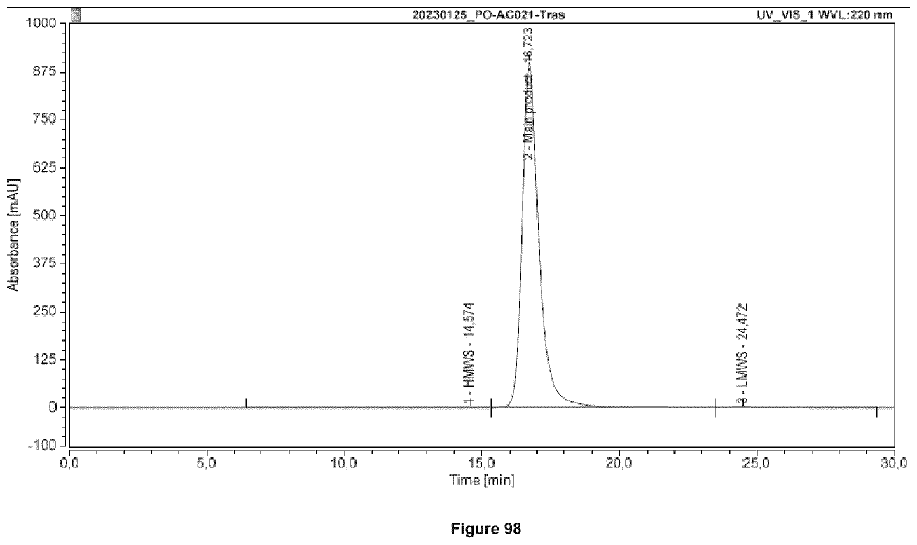

FIG. 98 shows an analytical SEC chromatogram of Trastuzumab-O—P5(PEG24)-amidopentyl-Phosphoramidate-N-(L-alanine-L-alanine)-O-Barasertib.

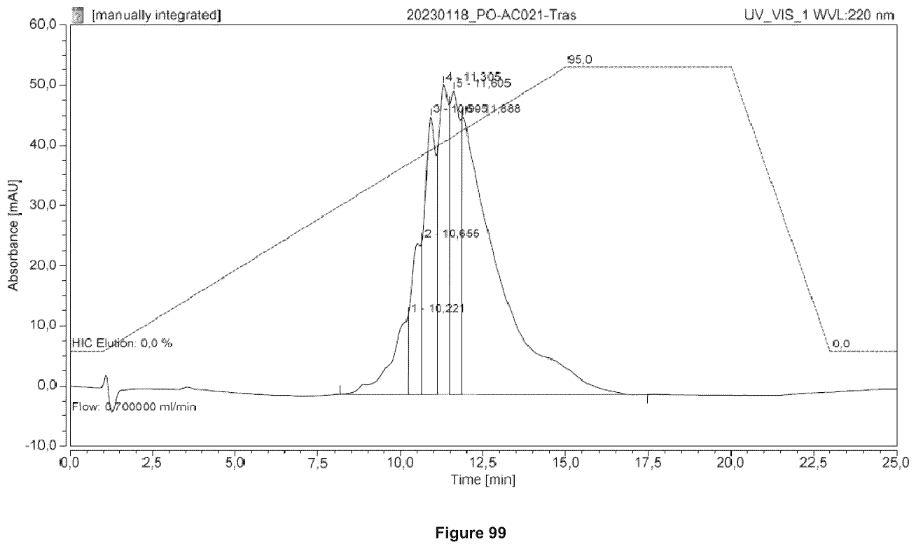

FIG. 99 shows an analytical HIC chromatogram of Trastuzumab-O—P5(PEG24)-amidopentyl-Phosphoramidate-N-(L-alanine-L-alanine)-O-Barasertib.

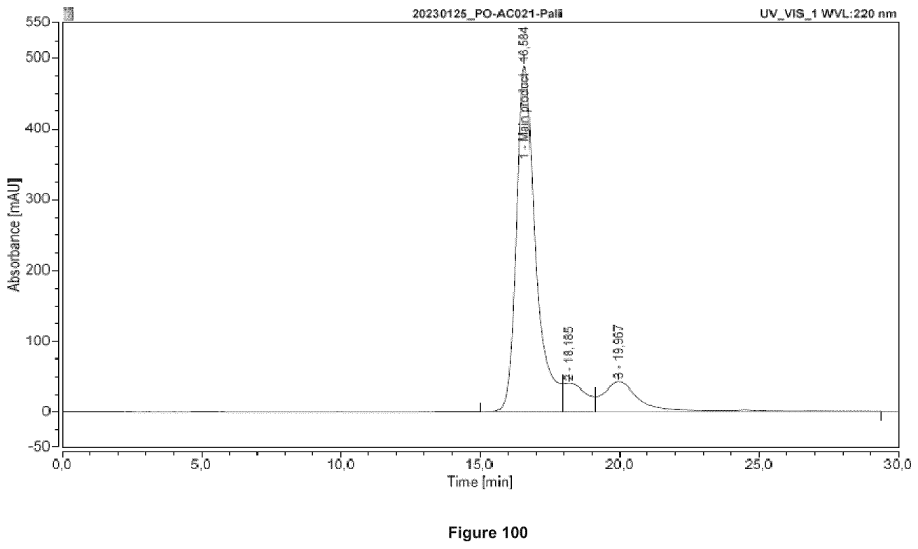

FIG. 100 shows an analytical SEC chromatogram of Palivizumab-O—P5(PEG24)-amidopentyl-Phosphoramidate-N-(L-alanine-L-alanine)-O-Barasertib.

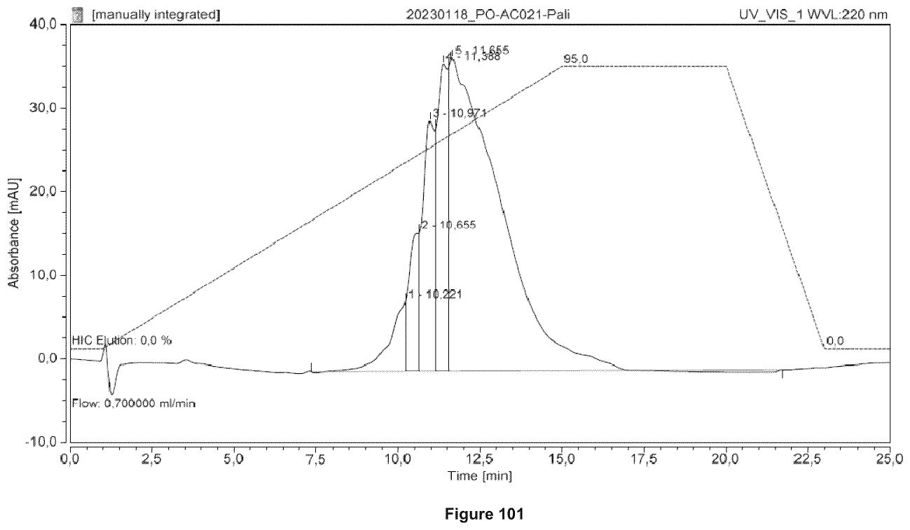

FIG. 101 shows an analytical HIC chromatogram of Palivizumab-O—P5(PEG24)-amidopentyl-Phosphoramidate-N-(L-alanine-L-alanine)-O-Barasertib.

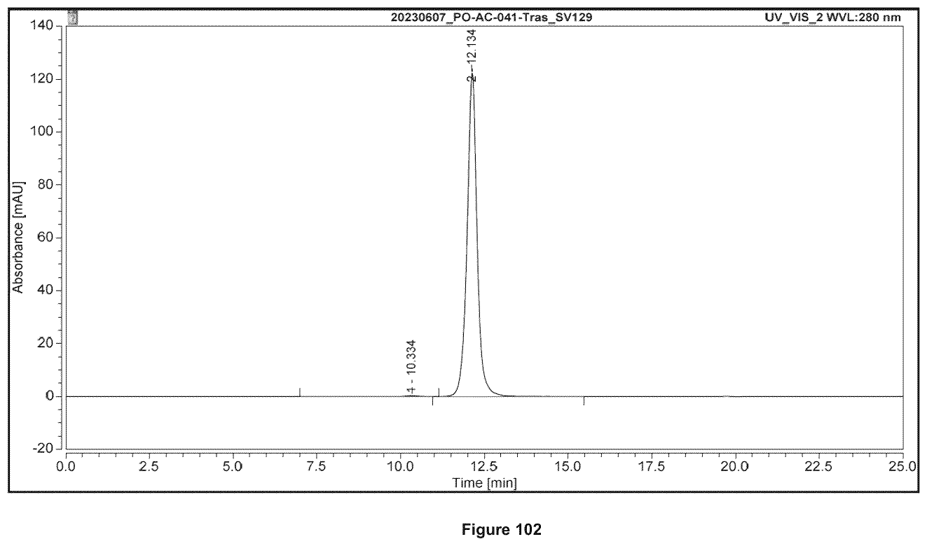

FIG. 102 shows an analytical SEC chromatogram of Trastuzumab-O—P5(PEG24)-amidopentyl-Phosphoramidate-N-(L-alanine-tert.-butylester)-O-Dxd.

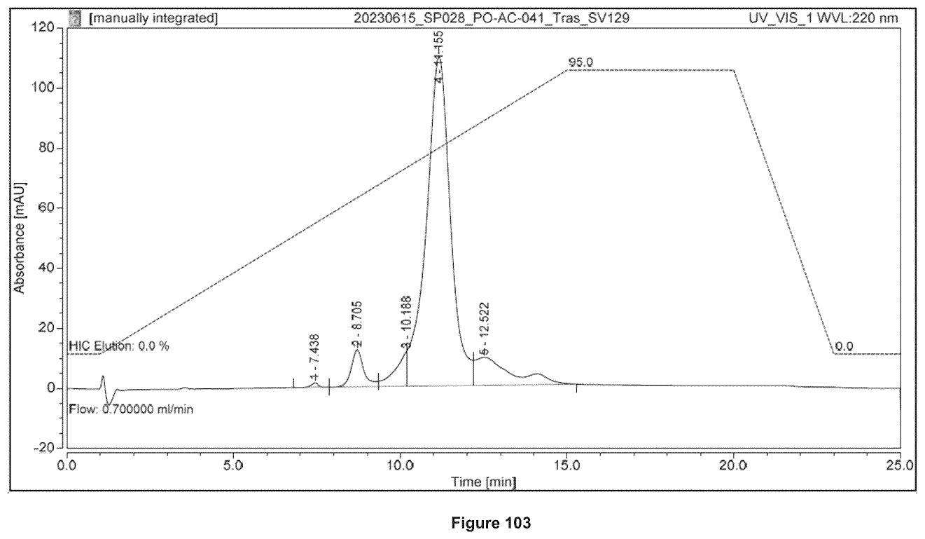

FIG. 103 shows an analytical HIC chromatogram of Trastuzumab-O—P5(PEG24)-amidopentyl-Phosphoramidate-N-(L-alanine-tert.-butylester)-O-Dxd.

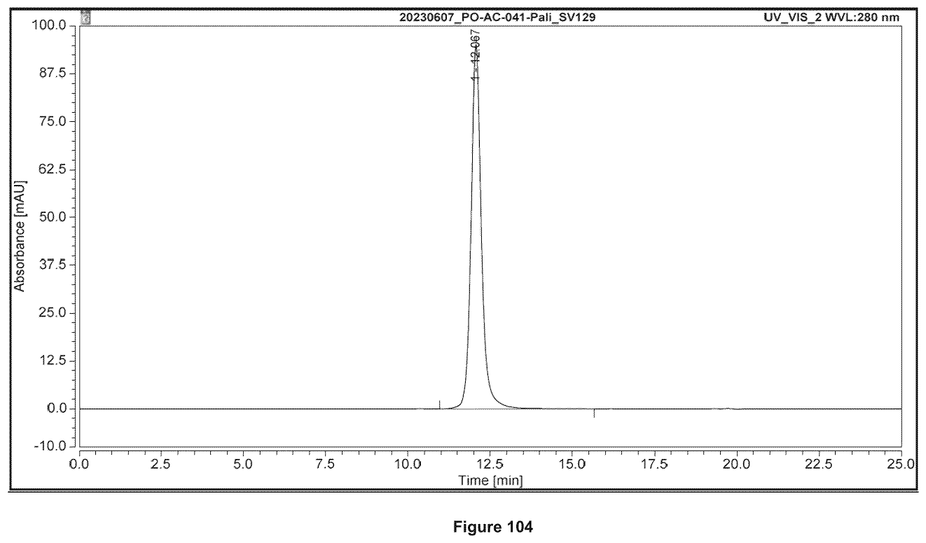

FIG. 104 shows an analytical SEC chromatogram of Palivizumab-O—P5(PEG24)-amidopentyl-Phosphoramidate-N-(L-alanine-tert.-butylester)-O-Dxd.

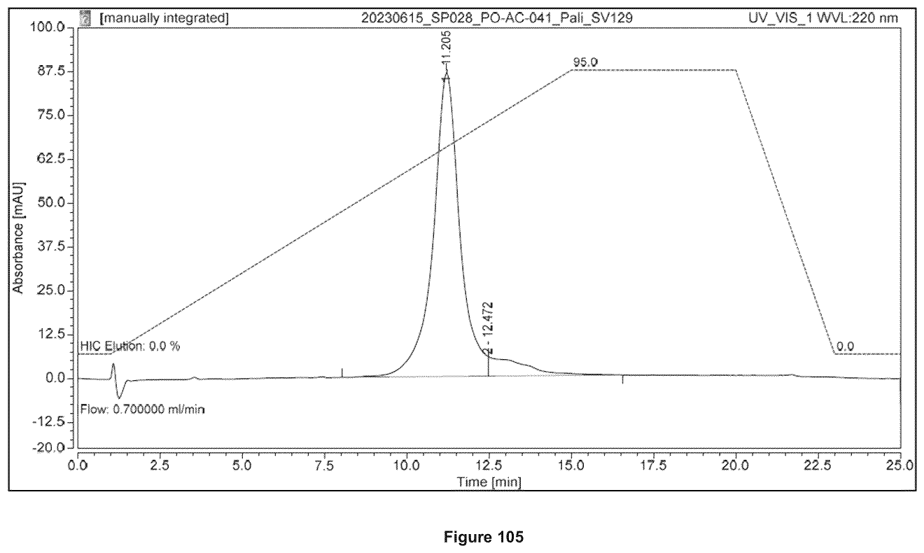

FIG. 105 shows an analytical HIC chromatogram of Palivizumab-O—P5(PEG24)-amidopentyl-Phosphoramidate-N-(L-alanine-tert.-butylester)-O-Dxd.

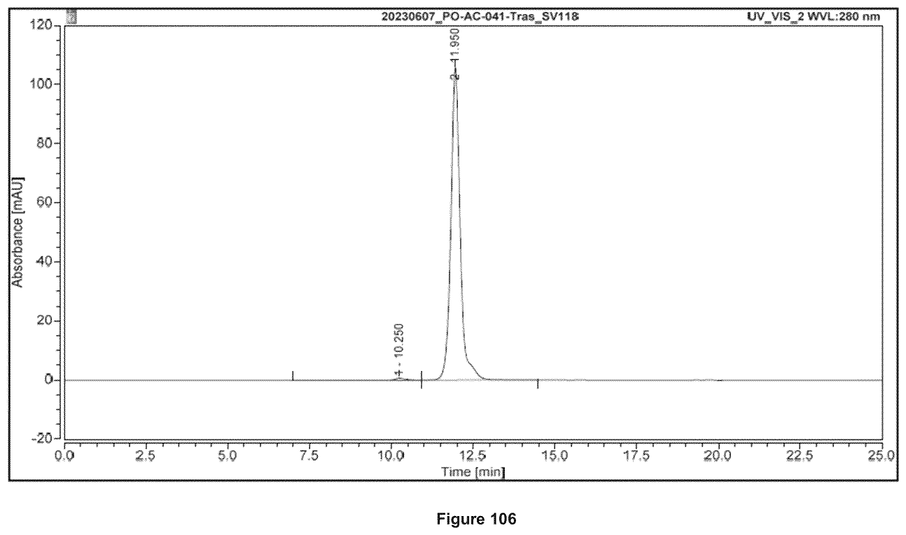

FIG. 106 shows an analytical SEC chromatogram of Trastuzumab-O—P5(PEG24)-amidopentyl-Phosphoramidate-N-(L-alanine-L-alanine)-O-Dxd.

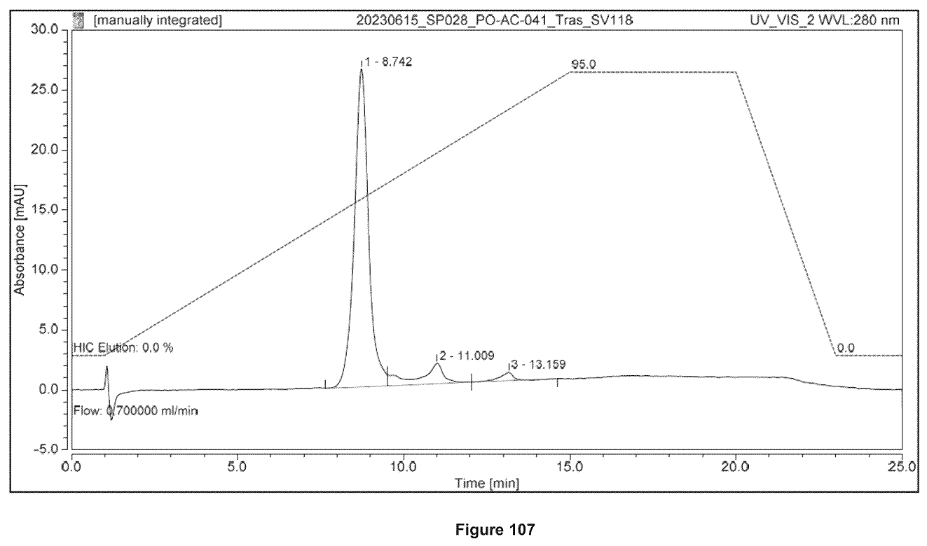

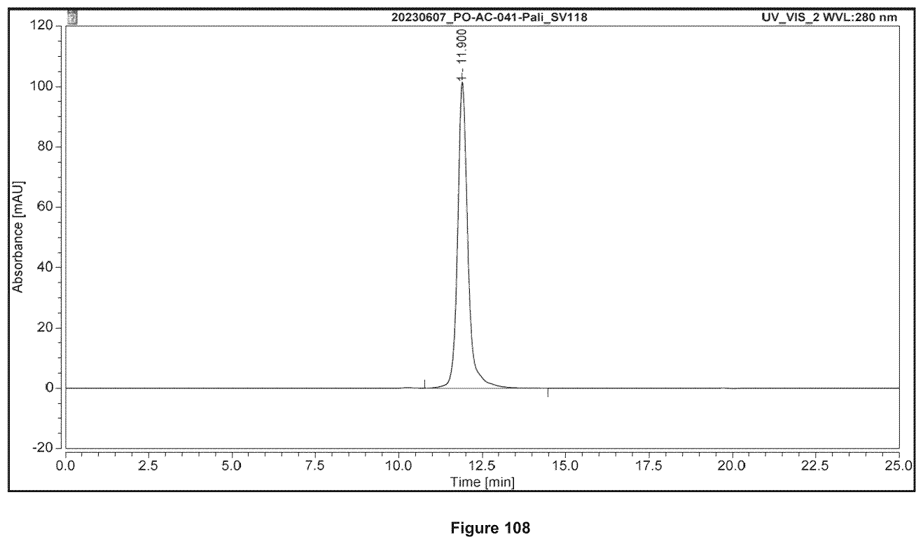

FIG. 107 shows an analytical HIC chromatogram of Trastuzumab-O—P5(PEG24)-amidopentyl-Phosphoramidate-N-(L-alanine-L-alanine)-O-Dxd.

FIG. 108 shows an analytical SEC chromatogram of Palivizumab-O—P5(PEG24)-amidopentyl-Phosphoramidate-N-(L-alanine-L-alanine)-O-Dxd.

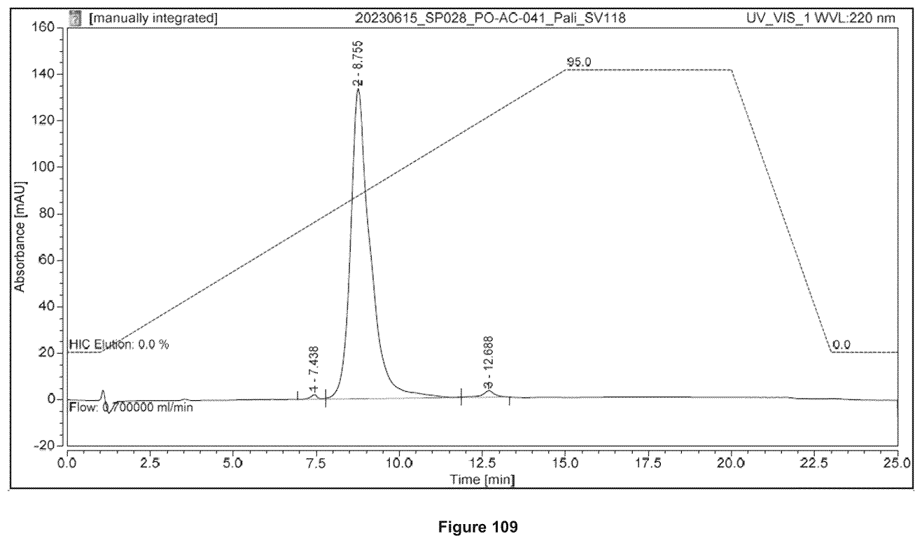

FIG. 109 shows an analytical HIC chromatogram of Palivizumab-O—P5(PEG24)-amidopentyl-Phosphoramidate-N-(L-alanine-L-alanine)-O-Dxd.

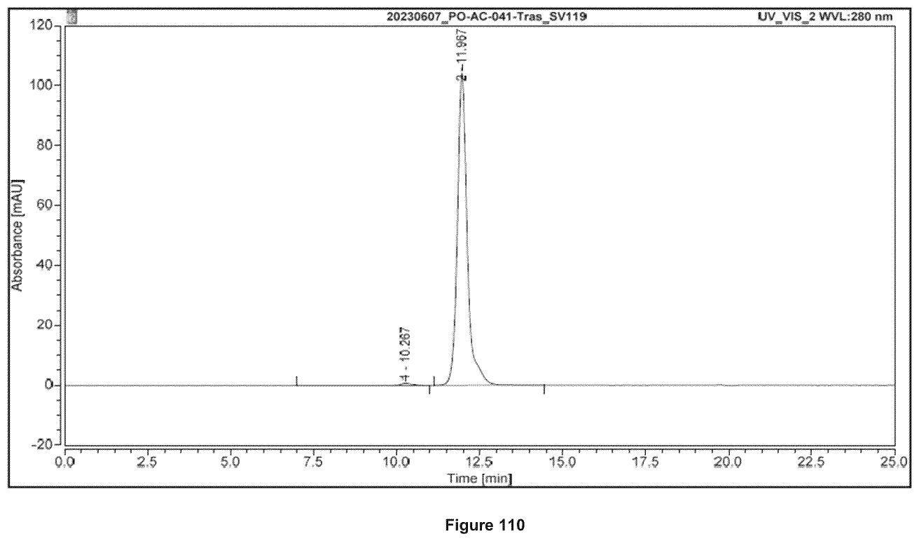

FIG. 110 shows an analytical SEC chromatogram of Trastuzumab-O—P5(PEG24)-amidopentyl-Phosphoramidate-N—(β-alanine-L-alanine)-O-Dxd.

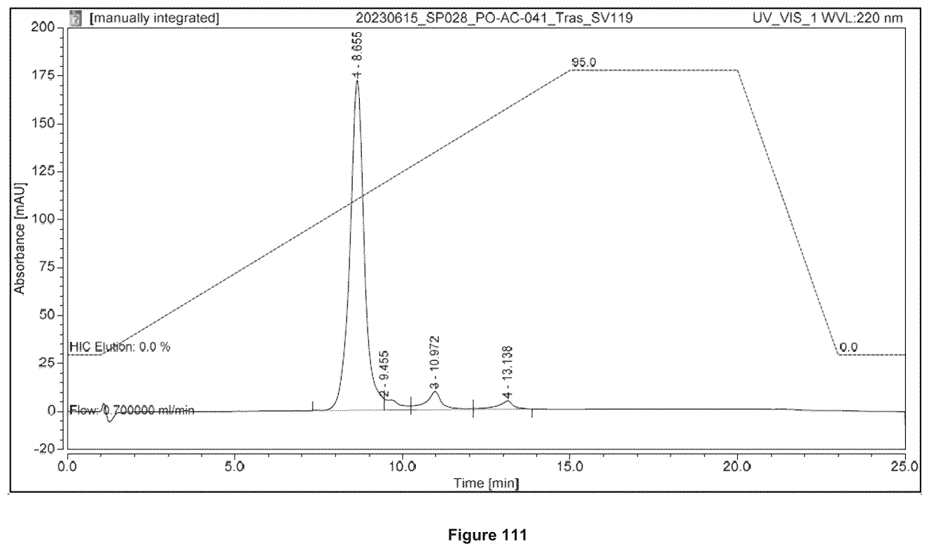

FIG. 111 shows an analytical HIC chromatogram of Trastuzumab-O—P5(PEG24)-amidopentyl-Phosphoramidate-N—(β-alanine-L-alanine)-O-Dxd.

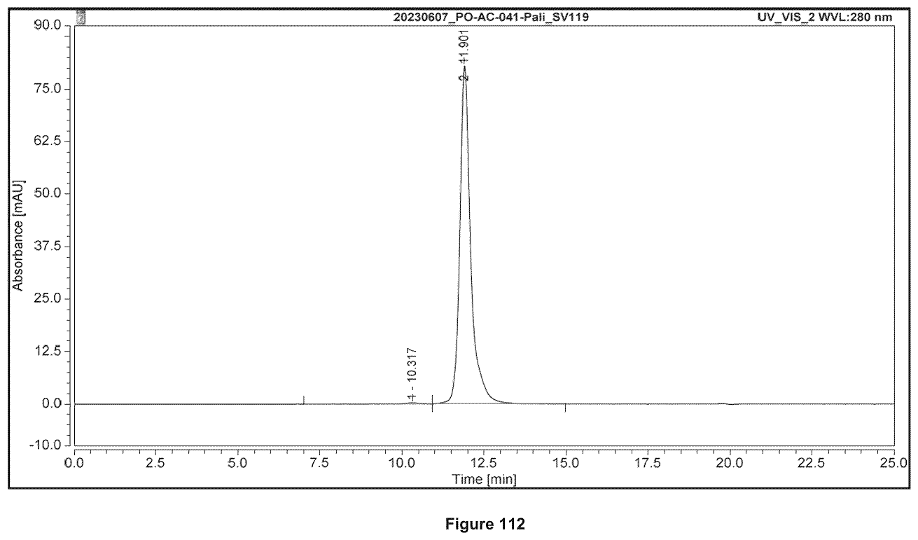

FIG. 112 shows an analytical SEC chromatogram of Palivizumab-O—P5(PEG24)-amidopentyl-Phosphoramidate-N—(β-alanine-L-alanine)-O-Dxd.

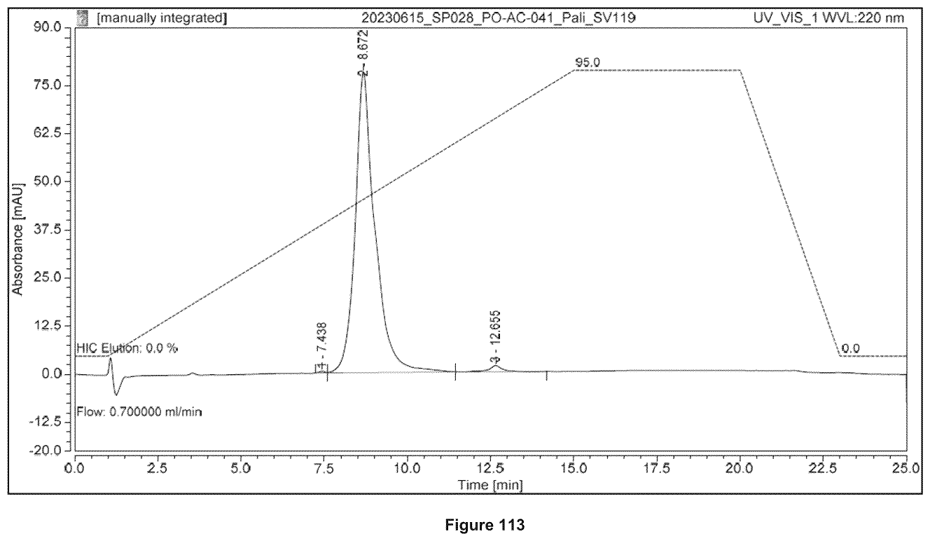

FIG. 113 shows an analytical HIC chromatogram of Palivizumab-O—P5(PEG24)-amidopentyl-Phosphoramidate-N—(β-alanine-L-alanine)-O-Dxd.

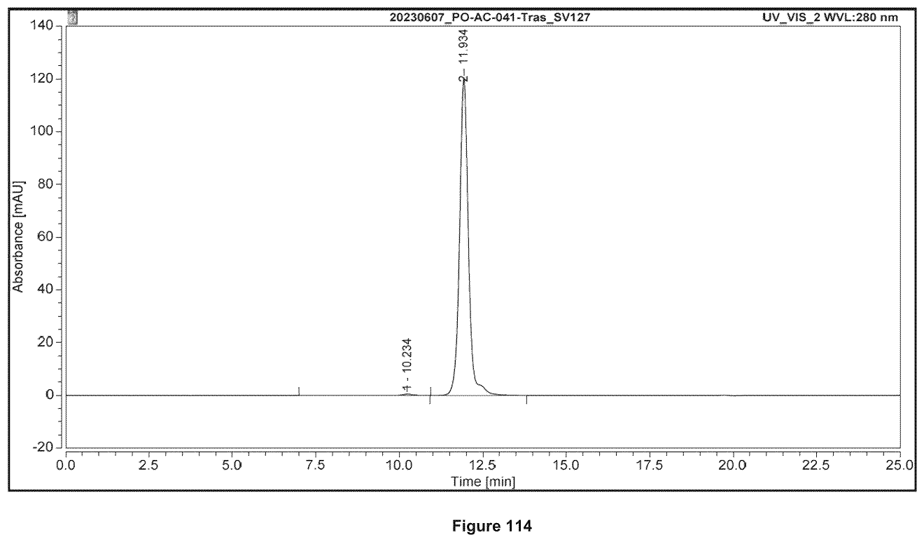

FIG. 114 shows an analytical SEC chromatogram of Trastuzumab-O—P5(PEG24)-amidopentyl-Phosphoramidate-N-(γ-Aminobutyric acid-L-alanine)-O-Dxd.

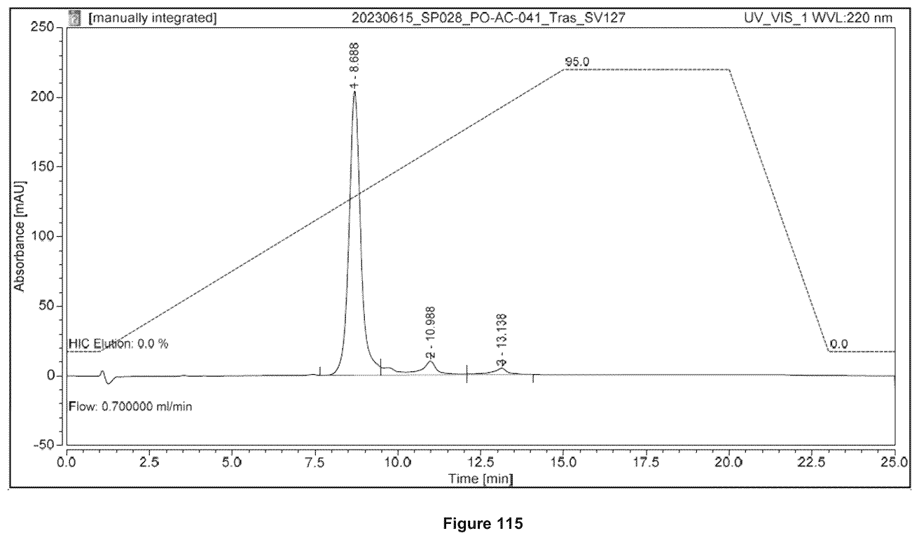

FIG. 115 shows an analytical HIC chromatogram of Trastuzumab-O—P5(PEG24)-amidopentyl-Phosphoramidate-N-(γ-Aminobutyric acid-L-alanine)-O-Dxd.

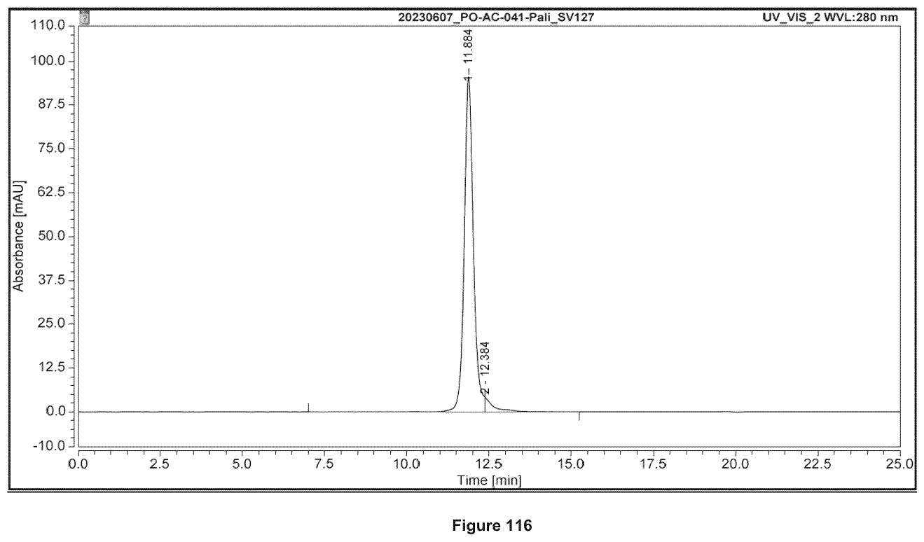

FIG. 116 shows an analytical SEC chromatogram of Palivizumab-O—P5(PEG24)-amidopentyl-Phosphoramidate-N-(γ-Aminobutyric acid-L-alanine)-O-Dxd.

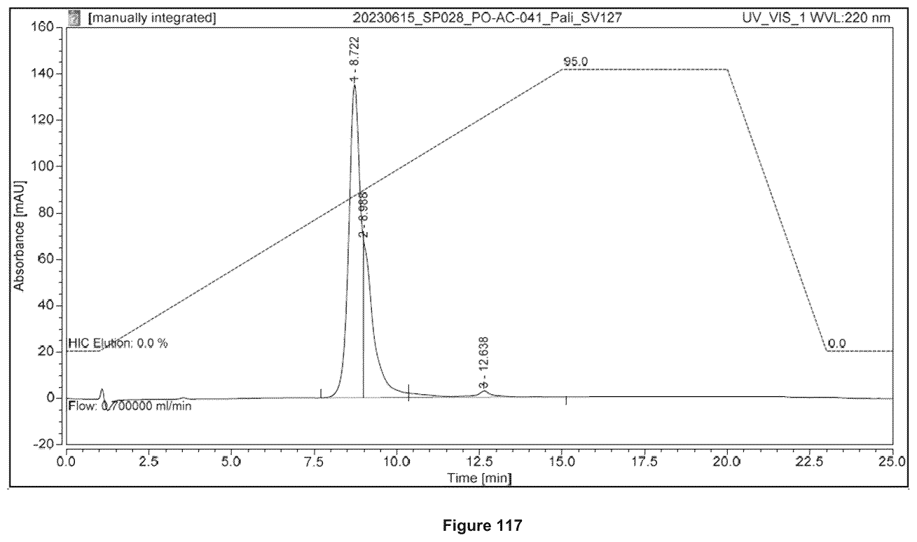

FIG. 117 shows an analytical HIC chromatogram of Palivizumab-O—P5(PEG24)-amidopentyl-Phosphoramidate-N-(γ-Aminobutyric acid-L-alanine)-O-Dxd.

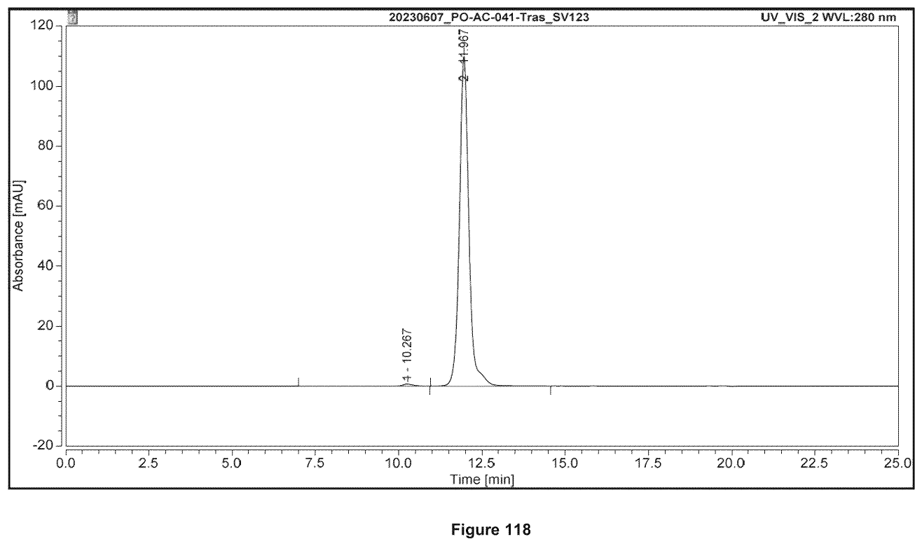

FIG. 118 shows an analytical SEC chromatogram of Trastuzumab-O—P5(PEG24)-amidopentyl-Phosphoramidate-N-(5-Aminovaleric acid-L-alanine)-O-Dxd.

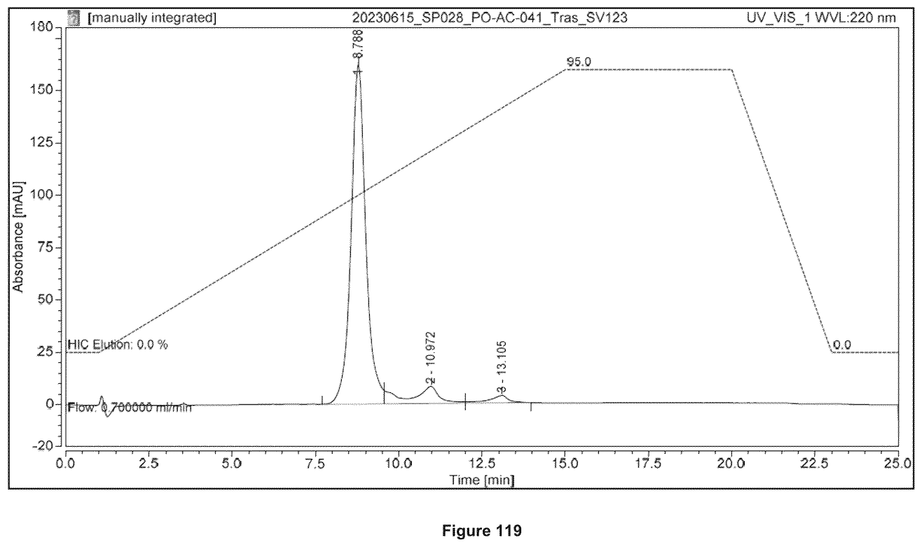

FIG. 119 shows an analytical HIC chromatogram of Trastuzumab-O—P5(PEG24)-amidopentyl-Phosphoramidate-N-(5-Aminovaleric acid-L-alanine)-O-Dxd.

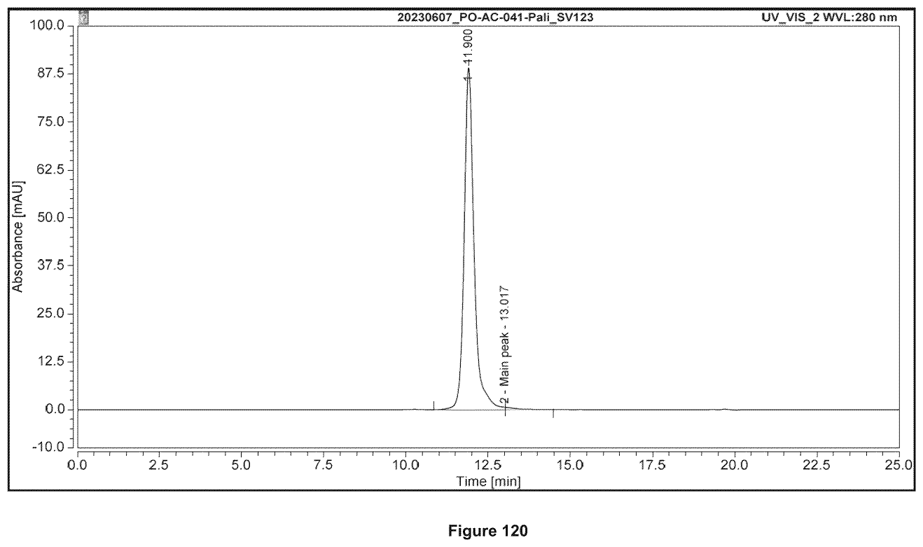

FIG. 120 shows an analytical SEC chromatogram of Palivizumab-O—P5(PEG24)-amidopentyl-Phosphoramidate-N-(5-Aminovaleric acid-L-alanine)-O-Dxd.

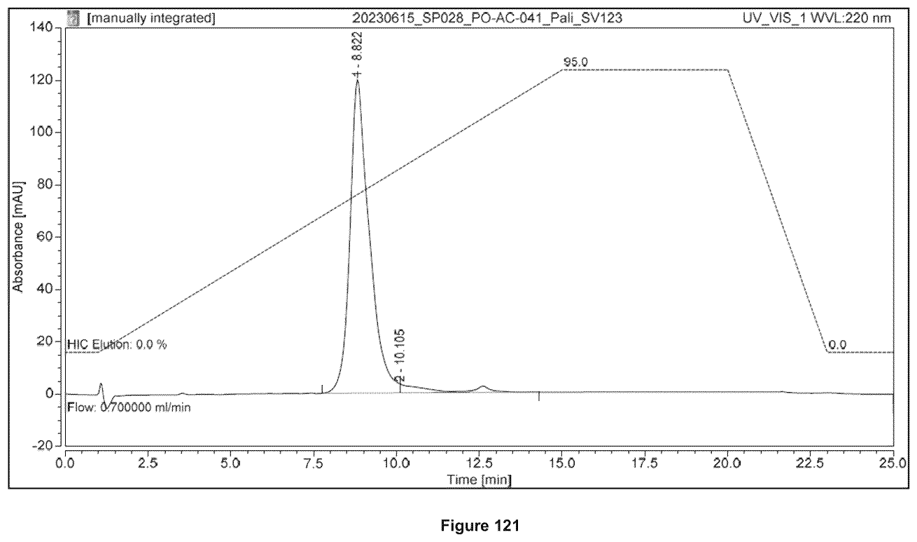

FIG. 121 shows an analytical HIC chromatogram of Palivizumab-O—P5(PEG24)-amidopentyl-Phosphoramidate-N-(5-Aminovaleric acid-L-alanine)-O-Dxd.

FIG. 189 shows an analytical SEC chromatogram of Brentuximab-O—P5(PEG24)-amidopentyl-Phosphoramidate-N-(L-alanine-iso-propylerster)-O—SN38

FIG. 190 shows an analytical HIC chromatogram of Brentuximab-O—P5(PEG24)-amidopentyl-Phosphoramidate-N-(L-alanine-iso-propylerster)-O—SN38

FIG. 191 shows an analytical SEC chromatogram of Datopotamab-O—P5(PEG24)-amidopentyl-Phosphoramidate-N-(L-alanine-iso-propylerster)-O—SN38

FIG. 192 shows an analytical HIC chromatogram of Datopotamab-O—P5(PEG24)-amidopentyl-Phosphoramidate-N-(L-alanine-iso-propylerster)-O—SN38

FIG. 193 shows an analytical SEC chromatogram of Brentuximab-O—P5(PEG24)-amidopentyl-Phosphoramidate-N-(glycine-tert.-butylester)-O—SN38

FIG. 194 shows an analytical HIC chromatogram of Brentuximab-O—P5(PEG24)-amidopentyl-Phosphoramidate-N-(glycine-tert.-butylester)-O—SN38

FIG. 195 shows an analytical SEC chromatogram of Datopotamab-O—P5(PEG24)-amidopentyl-Phosphoramidate-N-(glycine-tert.-butylester)-O—SN38

FIG. 196 shows an analytical HIC chromatogram of Datopotamab-O—P5(PEG24)-amidopentyl-Phosphoramidate-N-(glycine-tert.-butylester)-O—SN38

FIG. 197 shows an analytical SEC chromatogram of Brentuximab-O—P5(PEG24)-amidopentyl-Phosphoramidate-N-(2,2-dimethylaminobutyric acid-tert.-butylester)-O—SN38

FIG. 198 shows an analytical HIC chromatogram of Brentuximab-O—P5(PEG24)-amidopentyl-Phosphoramidate-N-(2,2-dimethylaminobutyric acid-tert.-butylester)-O—SN38

FIG. 199 shows an analytical SEC chromatogram of Datopotamab-O—P5(PEG24)-amidopentyl-Phosphoramidate-N-(2,2-dimethylaminobutyric acid-tert.-butylester)-O—SN38

FIG. 200 shows an analytical HIC chromatogram of Datopotamab-O—P5(PEG24)-amidopentyl-Phosphoramidate-N-(2,2-dimethylaminobutyric acid-tert.-butylester)-O—SN38

FIG. 201 shows an analytical SEC chromatogram of Brentuximab-O—P5(PEG24)-amidocyclohexyl-Phosphoramidate-N-(L-alanine-L-alanine)-O—SN38

FIG. 202 shows an analytical HIC chromatogram of Brentuximab-O—P5(PEG24)-amidocyclohexyl-Phosphoramidate-N-(L-alanine-L-alanine)-O—SN38

FIG. 203 shows an analytical SEC chromatogram of Datopotamab-O—P5(PEG24)-amidocyclohexyl-Phosphoramidate-N-(L-alanine-L-alanine)-O—SN38

FIG. 204 shows an analytical HIC chromatogram of Datopotamab-O—P5(PEG24)-amidocyclohexyl-Phosphoramidate-N-(L-alanine-L-alanine)-O—SN38

FIG. 205 shows an analytical SEC chromatogram of Brentuximab-O—P5(PEG24)-amidocyclohexyl-Phosphoramidate-N-(L-alanine-tert.-butylester)-O—SN38

FIG. 206 shows an analytical HIC chromatogram of Brentuximab-O—P5(PEG24)-amidocyclohexyl-Phosphoramidate-N-(L-alanine-tert.-butylester)-O—SN38

FIG. 207 shows an analytical SEC chromatogram of Datopotamab-O—P5(PEG24)-amidocyclohexyl-Phosphoramidate-N-(L-alanine-tert.-butylester)-O—SN38

FIG. 208 shows an analytical HIC chromatogram of Datopotamab-O—P5(PEG24)-amidocyclohexyl-Phosphoramidate-N-(L-alanine-tert.-butylester)-O—SN38

FIG. 209 shows an analytical SEC chromatogram of Brentuximab-O—P5(PEG24)-amidocyclohexyl-Phosphoramidate-N-(L-alanine-iso-propylester)-O—SN38

FIG. 210 shows an analytical HIC chromatogram of Brentuximab-O—P5(PEG24)-amidocyclohexyl-Phosphoramidate-N-(L-alanine-iso-propylester)-O—SN38

FIG. 211 shows an analytical SEC chromatogram of Datopotamab-O—P5(PEG24)-amidocyclohexyl-Phosphoramidate-N-(L-alanine-iso-propylester)-O—SN38

FIG. 212 shows an analytical HIC chromatogram of Datopotamab-O—P5(PEG24)-amidocyclohexyl-Phosphoramidate-N-(L-alanine-iso-propylester)-O—SN38

FIG. 213 shows an analytical SEC chromatogram of Brentuximab-5-Pentylacetamide-Phosphoramidate-N-(L-alanine-L-alanine)-O-DXd

FIG. 214 shows an analytical HIC chromatogram of Brentuximab-5-Pentylacetamide-Phosphoramidate-N-(L-alanine-L-alanine)-O-DXd

FIG. 215 shows an analytical SEC chromatogram of Datopotamab-O- Pentylacetamide-Phosphoramidate-N-(L-alanine-L-alanine)-O-DXd

FIG. 216 shows an analytical HIC chromatogram of Datopotamab-O— Pentylacetamide-Phosphoramidate-N-(L-alanine-L-alanine)-O-DXd

FIG. 217 shows an analytical SEC chromatogram of Brentuximab-6-(2,5-dioxopyrrolidin-1-yl)-N-(5-hydroxypentyl)hexanamido-Phosphoramidate-N-(L-alanine-L-alanine)-O-DXd

FIG. 218 shows an analytical HIC chromatogram of Brentuximab-6-(2,5-dioxopyrrolidin-1-yl)-N-(5-hydroxypentyl)hexanamido-Phosphoramidate-N-(L-alanine-L-alanine)-O-DXd

FIG. 219 shows an analytical SEC chromatogram of Datopotamab-6-(2,5-dioxopyrrolidin-1-yl)-N-(5-hydroxypentyl)hexanamido-Phosphoramidate-N-(L-alanine-L-alanine)-O-DXd

FIG. 220 shows an analytical HIC chromatogram of Datopotamab-6-(2,5-dioxopyrrolidin-1-yl)-N-(5-hydroxypentyl)hexanamido-Phosphoramidate-N-(L-alanine-L-alanine)-O-DXd

FIG. 221 shows an analytical SEC chromatogram of Brentuximab-O—P5(PEG24)-amidopentyl-Phosphoramidate-N-(L-alanine-L-alanine)-O-DHODH-IN-16

FIG. 222 shows an analytical HIC chromatogram of Brentuximab-O—P5(PEG24)-amidopentyl-Phosphoramidate-N-(L-alanine-L-alanine)-O-DHODH-IN-16

FIG. 223 shows an analytical SEC chromatogram of Datopotamab-O—P5(PEG24)-amidopentyl-Phosphoramidate-N-(L-alanine-L-alanine)-O-DHODH-IN-16

FIG. 224 shows an analytical HIC chromatogram of Datopotamab-O—P5(PEG24)-amidopentyl-Phosphoramidate-N-(L-alanine-L-alanine)-O-DHODH-IN-16

FIG. 225 shows an analytical SEC chromatogram of Brentuximab-O—P5(PEG24)-amidopentyl-Phosphoramidate-N-(L-alanine-L-alanine)-O-Roniciclib

FIG. 226 shows an analytical HIC chromatogram of Brentuximab-O—P5(PEG24)-amidopentyl-Phosphoramidate-N-(L-alanine-L-alanine)-O-Roniciclib

FIG. 227 shows an analytical SEC chromatogram of Trastuzumab-O—P5(PEG24)-amidopentyl-Phosphoramidate-N-(L-alanine-L-alanine)-O-Roniciclib

FIG. 228 shows an analytical HIC chromatogram of Trastuzumab-O—P5(PEG24)-amidopentyl-Phosphoramidate-N-(L-alanine-L-alanine)-O-Roniciclib

FIG. 229 shows an analytical SEC chromatogram of Brentuximab-O—P5(PEG24)-amidopentyl-Phosphoramidate-N-(L-alanine-L-alanine)-O-Nampt-IN-1

FIG. 230 shows an analytical HIC chromatogram of Brentuximab-O—P5(PEG24)-amidopentyl-Phosphoramidate-N-(L-alanine-L-alanine)-O-Nampt-IN-1

FIG. 231 shows an analytical SEC chromatogram of Datopotamab-O—P5(PEG24)-amidopentyl-Phosphoramidate-N-(L-alanine-L-alanine)-O-Nampt-IN-1

FIG. 232 shows an analytical HIC chromatogram of Datopotamab-O—P5(PEG24)-amidopentyl-Phosphoramidate-N-(L-alanine-L-alanine)-O-Nampt-IN-1

FIG. 233 shows an analytical SEC chromatogram of Brentuximab-O—P5(PEG24)-amidopentyl-Phosphoramidate-N-(L-alanine-L-alanine-tert.-butylester)-O-Paclitaxel

FIG. 234 shows an analytical HIC chromatogram of Brentuximab-O—P5(PEG24)-amidopentyl-Phosphoramidate-N-(L-alanine-L-alanine-tert.-butylester)-O-Paclitaxel

FIG. 235 shows an analytical SEC chromatogram of Trastuzumab-O—P5(PEG24)-amidopentyl-Phosphoramidate-N-(L-alanine-L-alanine-tert.-butylester)-O-Paclitaxel

FIG. 236 shows an analytical HIC chromatogram of Trastuzumab-O—P5(PEG24)-amidopentyl-Phosphoramidate-N-(L-alanine-L-alanine-tert.-butylester)-O-Paclitaxel

FIG. 237 shows an analytical SEC chromatogram of Brentuximab-O—P5(PEG24)-amidopentyl-Phosphoramidate-N-(L-alanine-L-alanine-tert.-butylester)-O-Triptolide

FIG. 238 shows an analytical HIC chromatogram of Brentuximab-O—P5(PEG24)-amidopentyl-Phosphoramidate-N-(L-alanine-L-alanine-tert.-butylester)-O-Triptolide

FIG. 239 shows an analytical SEC chromatogram of Trastuzumab-O—P5(PEG24)-amidopentyl-Phosphoramidate-N-(L-alanine-L-alanine-tert.-butylester)-O-Triptolide

FIG. 240 shows an analytical HIC chromatogram of Trastuzumab-O—P5(PEG24)-amidopentyl-Phosphoramidate-N-(L-alanine-L-alanine-tert.-butylester)-O-Triptolide

FIG. 241 shows an analytical SEC chromatogram of Brentuximab-O—P5(PEG24)-amidopentyl-Phosphoramidate-N-(L-alanine-L-alanine-)—O—ON013100

FIG. 242 shows an analytical HIC chromatogram of Brentuximab-O—P5(PEG24)-amidopentyl-Phosphoramidate-N-(L-alanine-L-alanine-)—O—ON013100

FIG. 243 shows an analytical SEC chromatogram of Datopotamab-O—P5(PEG24)-amidopentyl-Phosphoramidate-N-(L-alanine-L-alanine-)—O—ON013100

FIG. 244 shows an analytical HIC chromatogram of Datopotamab-O—P5(PEG24)-amidopentyl-Phosphoramidate-N-(L-alanine-L-alanine-)—O—ON013100

FIG. 245 shows an analytical SEC chromatogram of Brentuximab-O—P5(PEG24)-amidopentyl-Phosphoramidate-N-(L-alanine-L-alanine)-O-Ganetespib

FIG. 246 shows an analytical HIC chromatogram of Brentuximab-O—P5(PEG24)-amidopentyl-Phosphoramidate-N-(L-alanine-L-alanine)-O-Ganetespib

FIG. 247 shows an analytical SEC chromatogram of Datopotamab-O—P5(PEG24)-amidopentyl-Phosphoramidate-N-(L-alanine-L-alanine)-O-Ganetespib

FIG. 248 shows an analytical HIC chromatogram of Datopotamab-O—P5(PEG24)-amidopentyl-Phosphoramidate-N-(L-alanine-L-alanine)-O-Ganetespib.

FIG. 250 shows an analytical SEC chromatogram of Brentuximab.

FIG. 251 shows an analytical HIC chromatogram of Brentuximab.

FIG. 252 shows an analytical SEC chromatogram of Datopotamab.

FIG. 253 shows an analytical HIC chromatogram of Datopotamab.

FIG. 256 shows an analytical SEC chromatogram of Brentuximab-O—P5(PEG24)-amidopentyl-Phosphoramidate-N-(L-alanine-tert.-butylerster)-O—SN38.

FIG. 257 shows an analytical HIC chromatogram of Brentuximab-O—P5(PEG24)-amidopentyl-Phosphoramidate-N-(L-alanine-tert.-butylerster)-O—SN38.

FIG. 258 shows an analytical SEC chromatogram of Datopotamab-O—P5(PEG24)-amidopentyl-Phosphoramidate-N-(L-alanine-tert.-butylerster)-O—SN38.

FIG. 259 shows an analytical HIC chromatogram of Datopotamab-O—P5(PEG24)-amidopentyl-Phosphoramidate-N-(L-alanine-tert.-butylerster)-O—SN38.

FIG. 260 shows an analytical SEC chromatogram of Trastuzumab-O—P5(PEG24)-amidopentyl-Phosphoramidate-N-(L-alanine-L-alanine)-O-Ganetespib.

FIG. 261 shows an analytical HIC chromatogram of Trastuzumab-O—P5(PEG24)-amidopentyl-Phosphoramidate-N-(L-alanine-L-alanine)-O-Ganetespib.

FIG. 262 shows an analytical SEC chromatogram of Palivizumab-O-P5-(PEG24)-amidopentyl-Phosphoramidate-N-(L-alanine-L-alanine)-O-BAY-2402234.

FIG. 263 shows an analytical HIC chromatogram of Palivizumab-O-P5-(PEG24)-amidopentyl-Phosphoramidate-N-(L-alanine-L-alanine)-O-BAY-2402234.

FIG. 264 shows an analytical SEC chromatogram of Datopotamab-O-P5-(PEG24)-amidopentyl-Phosphoramidate-N-(L-alanine-L-alanine)-O-BAY-2402234.

FIG. 265 shows an analytical HIC chromatogram of Datopotamab-O-P5-(PEG24)-amidopentyl-Phosphoramidate-N-(L-alanine-L-alanine)-O-BAY-2402234.

FIG. 284 shows an analytical SEC chromatogram of Brentuximab-O—P5(PEG24)-amidopentyl-Phosphoramidate-N-(2-tert.-butyl-disulfide-ethyl)-O—SN38.

FIG. 285 shows an analytical HIC chromatogram of Brentuximab-O—P5(PEG24)-amidopentyl-Phosphoramidate-N-(2-tert.-butyl-disulfide-ethyl)-O—SN38.

FIG. 286 shows an analytical SEC chromatogram of Datopotamab-O—P5(PEG24)-amidopentyl-Phosphoramidate-N-(2-tert.-butyl-disulfide-ethyl)-O—SN38.

FIG. 287 shows an analytical HIC chromatogram of Datopotamab-O—P5(PEG24)-amidopentyl-Phosphoramidate-N-(2-tert.-butyl-disulfide-ethyl)-O—SN38.

FIG. 288 shows an analytical SEC chromatogram of Brentuximab-O—P5(PEG24)-amidopentyl-Phosphoramidate-N-(2-tert.-butyl-disulfide-ethyl)-O-DXD.

FIG. 289 shows an analytical HIC chromatogram of Brentuximab-O—P5(PEG24)-amidopentyl-Phosphoramidate-N-(2-tert.-butyl-disulfide-ethyl)-O-DXD.

FIG. 290 shows an analytical SEC chromatogram of O—P5(PEG24)-amidopentyl-Phosphoramidate-N-(2-tert.-butyl-disulfide-ethyl)-O-DXD.

FIG. 291 shows an analytical HIC chromatogram of O—P5(PEG24)-amidopentyl-Phosphoramidate-N-(2-tert.-butyl-disulfide-ethyl)-O-DXD.

FIG. 292 shows an analytical SEC chromatogram of Brentuximab-O—P5(PEG24)-amidopentyl-Phosphoramidate-N-(2-ethoxy-6-(3R,4S,5S,6S)-(methoxycarbonyl)tetrahydro-2H-pyran-3,4,5-triyl triacetate)-O—SN38.

FIG. 293 shows an analytical HIC chromatogram of Brentuximab-O—P5(PEG24)-amidopentyl-Phosphoramidate-N-(2-ethoxy-6-(3R,4S,5S,6S)-(methoxycarbonyl)tetrahydro-2H-pyran-3,4,5-triyl triacetate)-O—SN38.

FIG. 294 shows an analytical SEC chromatogram of Datopotamab-O—P5(PEG24)-amidopentyl-Phosphoramidate-N-(2-ethoxy-6-(3R,4S,5S,6S)-(methoxycarbonyl)tetrahydro-2H-pyran-3,4,5-triyl triacetate)-O—SN38.

FIG. 295 shows an analytical HIC chromatogram of Datopotamab-O—P5(PEG24)-amidopentyl-Phosphoramidate-N-(2-ethoxy-6-(3R,4S,5S,6S)-(methoxycarbonyl)tetrahydro-2H-pyran-3,4,5-triyl triacetate)-O—SN38.

FIG. 296 shows an analytical SEC chromatogram of Brentuximab-O—P5(PEG24)-amidopentyl-Phosphoramidate-N-(2-Acetoxy-ethyl)-O-DXD.

FIG. 297 shows an analytical HIC chromatogram of Brentuximab-O—P5(PEG24)-amidopentyl-Phosphoramidate-N-(2-Acetoxy-ethyl)-O-DXD.

FIG. 298 shows an analytical SEC chromatogram of Datopotamab-O—P5(PEG24)-amidopentyl-Phosphoramidate-N-(2-Acetoxy-ethyl)-O-DXD.

FIG. 299 shows an analytical HIC chromatogram of Datopotamab-O—P5(PEG24)-amidopentyl-Phosphoramidate-N-(2-Acetoxy-ethyl)-O-DXD.

FIG. 300 shows an analytical SEC chromatogram of Brentuximab-O—P5(PEG24)-amidopentyl-Phosphoramidate-N-(2-Acetamido-ethyl)-O—SN38.

FIG. 301 shows an analytical HIC chromatogram of Brentuximab-O—P5(PEG24)-amidopentyl-Phosphoramidate-N-(2-Acetamido-ethyl)-O—SN38.

FIG. 302 shows an analytical SEC chromatogram of Datopotamab-O—P5(PEG24)-amidopentyl-Phosphoramidate-N-(2-Acetamido-ethyl)-O—SN38.

FIG. 303 shows an analytical HIC chromatogram of Datopotamab-O—P5(PEG24)-amidopentyl-Phosphoramidate-N-(2-Acetamido-ethyl)-O—SN38.

FIG. 304 shows an analytical SEC chromatogram of Brentuximab-O-P5-(PEG24)-amidopentyl-Phosphoramidate-O-(lactic acid-iso-propylester)-O—SN38.

FIG. 305 shows an analytical HIC chromatogram of Brentuximab-O-P5-(PEG24)-amidopentyl-Phosphoramidate-O-(lactic acid-iso-propylester)-O—SN38.

FIG. 306 shows an analytical SEC chromatogram of Datopotamab-O-P5-(PEG24)-amidopentyl-Phosphoramidate-O-(lactic acid-iso-propylester)-O—SN38.

FIG. 307 shows an analytical HIC chromatogram of Datopotamab-O-P5-(PEG24)-amidopentyl-Phosphoramidate-O-(lactic acid-iso-propylester)-O—SN38.

FIG. 308 shows an analytical SEC chromatogram of Brentuximab-O-P5-(PEG24)-amidopentyl-Phosphoramidate-O-(glycolic acid-iso-propylester)-N-Exatecan.

FIG. 309 shows an analytical HIC chromatogram of Brentuximab-O-P5-(PEG24)-amidopentyl-Phosphoramidate-O-(glycolic acid-iso-propylester)-N-Exatecan.

FIG. 310 shows an analytical SEC chromatogram of Datopotamab-O-P5-(PEG24)-amidopentyl-Phosphoramidate-O-(glycolic acid-iso-propylester)-N-Exatecan.

FIG. 311 shows an analytical HIC chromatogram of Datopotamab-O-P5-(PEG24)-amidopentyl-Phosphoramidate-O-(glycolic acid-iso-propylester)-N-Exatecan.

FIG. 312 shows an analytical SEC chromatogram of Trastuzumab-O—P5(PEG24)-amidopentyl-Phosphoramidate-N-(L-alanine-L-alanine-)—O—ON013100.

FIG. 313 shows an analytical HIC chromatogram of Trastuzumab-O—P5(PEG24)-amidopentyl-Phosphoramidate-N-(L-alanine-L-alanine-)—O—ON013100.

FIG. 314 shows an analytical SEC chromatogram of Brentuximab-O—P5(PEG24)-amidopentyl-Phosphoramidate-N-(L-alanine-iso-propylester)-O—ON013100.

FIG. 315 shows an analytical HIC chromatogram of Brentuximab-O—P5(PEG24)-amidopentyl-Phosphoramidate-N-(L-alanine-iso-propylester)-O—ON013100.

FIG. 316 shows an analytical SEC chromatogram of Datopotamab-O—P5(PEG24)-amidopentyl-Phosphoramidate-N-(L-alanine-iso-propylester)-O—ON013100.

FIG. 317 shows an analytical HIC chromatogram of Datopotamab-O—P5(PEG24)-amidopentyl-Phosphoramidate-N-(L-alanine-iso-propylester)-O—ON013100.

FIG. 318 shows an analytical SEC chromatogram of Trastuzumab-O—P5(PEG24)-amidopentyl-Phosphoramidate-N-(L-alanine-iso-propylester)-O—ON013100.

FIG. 319 shows an analytical HIC chromatogram of Trastuzumab-O—P5(PEG24)-amidopentyl-Phosphoramidate-N-(L-alanine-iso-propylester)-O—ON013100.

FIG. 320 shows an analytical SEC chromatogram of Brentuximab-O—P5(PEG24)-amidopentyl-Phosphoramidate-N-(L-alanine-isopropylester)-O-Ganetespib.

FIG. 321 shows an analytical HIC chromatogram of Brentuximab-O—P5(PEG24)-amidopentyl-Phosphoramidate-N-(L-alanine-isopropylester)-O-Ganetespib.

FIG. 322 shows an analytical SEC chromatogram of Datopotamab-O—P5(PEG24)-amidopentyl-Phosphoramidate-N-(L-alanine-isopropylester)-O-Ganetespib.

FIG. 323 shows an analytical HIC chromatogram of Datopotamab-O—P5(PEG24)-amidopentyl-Phosphoramidate-N-(L-alanine-isopropylester)-O-Ganetespib.

### Example 3: In Vitro Cell Viability

### SN38 Based ADCs

Antibody-drug conjugates (ADCs) comprising the anti-Trop2 antibody Sacituzumab, a phosphorus(V) moiety according to embodiments of the present invention, and SN38 as drug were tested in in vitro potency assays against various Trop2-positive cell lines. The corresponding ADCs with the non-targeted antibody Trastuzumab were used as isotype control. For direct comparison, the approved and marketed ADC Trodelvy comprising Sacituzumab, a CL2A linker and SN38 as drug was also tested. The corresponding ADC with Trastuzumab was used as isotype control.

FIGS. 45A and 45B show the results of the direct comparison between ADCs from O—P5(PEG12)-amidopentyl-Phosphoramidate-N-(L-alanine-tert.-butylester)-O—SN38 in accordance with embodiments of the present invention (FIG. 45A) and ADCs from CL2A-SN38, which is used in Trodelvy (FIG. 45B). Linkers have been conjugated to Sacituzumab (anti-Trop2) and Trastuzumab (anti Her2, isotype in this setting) and tested on a panel of 4 different cell lines. All cell lines were Trop+ and Her2−. Trastuzumab conjugates serve as isotype control in this setting. FIG. 45A shows the conjugates from O—P5(PEG12)-amidopentyl-Phosphoramidate-N-(L-alanine-tert.-butylester)-O—SN38. FIG. 45B shows the conjugates from CL2A-SN38. Solid lines are Sacituzumab conjugates and dotted lines are Trastuzumab conjugates. Sacituzumab-CL2A-SN38 is Trodelvy and has been purchased for the experiment. Trastuzumab-CL2A-SN38, having a drug to antibody ratio of 8 (DAR8), has been prepared according to a known procedure using the commercially available CL2A linker for the purpose of these experiments (analytical data not shown).
In these experiments, Trastuzumab-O—P5(PEG12)-amidopentyl-Phosphoramidate-N-(L-alanine-tert.-butylester)-O—SN38 having a drug to antibody ratio of 8 (DAR8) and Sacituzumab-O—P5(PEG12)-amidopentyl-Phosphoramidate-N-(L-alanine-tert.-butylester)-O—SN38 having a drug to antibody ratio of 8 (DAR8) have been tested in in vitro potency assays in direct comparison to Trodelvy.
No difference was observed for the in vitro potency for the targeted ADC comprising Sacituzumab and a CL2A linker (Trodelvy, Sacituzumab-CL2A-SN38 DAR8) compared to the non-targeted ADC comprising Trastuzumab with the same linker payload and DAR (Trastuzumab-CL2A-SN38 DAR8) on all 4 different cell lines treated. This is in line with the observations reported in S. V Govindan et al., “Improving the Therapeutic Index in Cancer Therapy by Using Antibody-Drug Conjugates Designed with a Moderately Cytotoxic Drug”, Mol. Pharmaceutics 2015, 12, 6, 1836-1847, doi.org/10.1021/mp5006195. Cleavage of the conjugate to release the drug under the assay conditions prior to specifically bind the target caused the potency of the conjugates to be very similar to that of the free drug. This observation demonstrates the instability of the CL2A under the in vitro cell viability assay conditions.
In contrast, conjugates in accordance with embodiments of the invention from compound O—P5(PEG12)-amidopentyl-Phosphoramidate-N-(L-alanine-tert.-butylester)-O—SN38 and targeted Sacituzumab (DAR8) showed a much higher potency compared to the conjugates comprising non-targeted Trastuzumab (DAR8) that served as isotype control in this setting. Thus, conjugates in accordance with the present invention enable targeted delivery of SN38 to Trop2-positive cells that is mediated by the specificity of the antibody. Moreover, this observation underlines the high stability of the conjugates in accordance with embodiments of the invention.

The inventors also tested the in vitro potency of a conjugate in accordance with an embodiment of the invention (Sacituzumab conjugated to the cleavable O—P5(PEG12)-amidopentyl-Phosphoramidate-N-(L-alanine-tert.-butylester)-O—SN38) and a conjugate comprising a non-cleavable side chain on the phosphorus(V) moiety (Sacituzumab conjugated to O—P5(PEG12)-amidopentyl-Phosphoramidate-N-(4-Methylbenzyl)-O—SN38) against various target-positive cell lines.

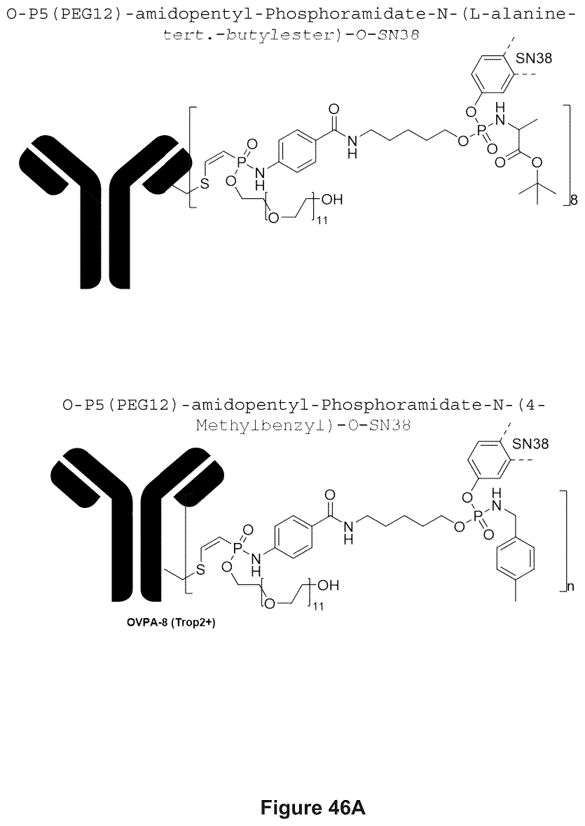

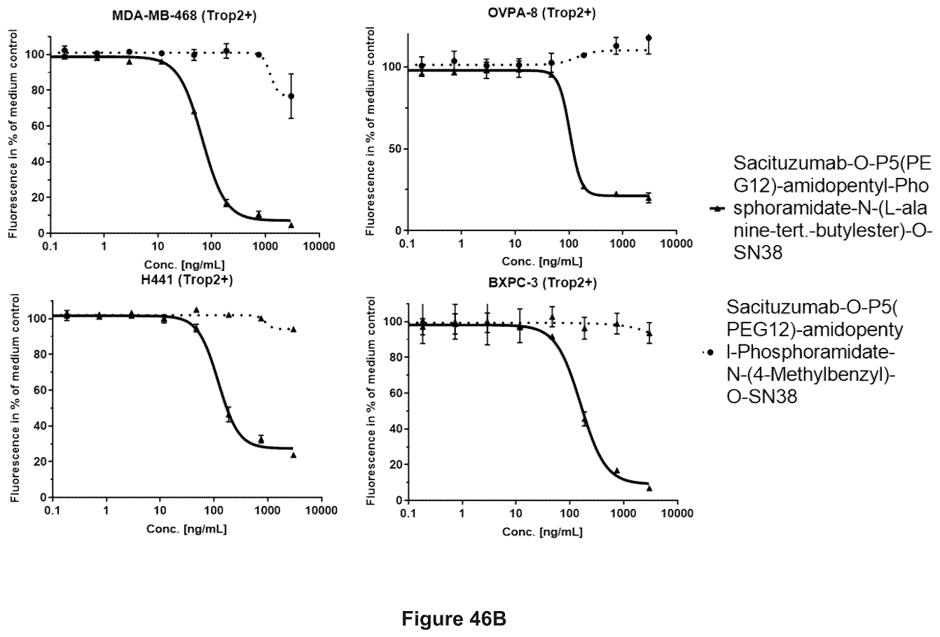

FIGS. 46A and 46B show the in vitro potency of an ADC comprising Sacituzumab conjugated to the cleavable O—P5(PEG12)-amidopentyl-Phosphoramidate-N-(L-alanine-tert.-butylester)-O—SN38 (solid) in accordance with an embodiment of the invention, and the in vitro potency of an ADC comprising Sacituzumab conjugated to the non cleavable control O—P5(PEG12)-amidopentyl-Phosphoramidate-N-(4-Methylbenzyl)-O—SN38 (dashed).
A clear discrepancy in potency between the ADC comprising Sacituzumab conjugated to O—P5(PEG12)-amidopentyl-Phosphoramidate-N-(L-alanine-tert.-butylester)-O—SN38 (in accordance with an embodiment of the invention) in direct comparison to the ADC comprising Sacituzumab conjugated to O—P5(PEG12)-amidopentyl-Phosphoramidate-N-(4-Methylbenzyl)-O—SN38 was observed.
The absence of potency in the non-cleavable control clearly underlines the release of the drug (SN38) in dependence of the intracellular liberation of a nucleophile (a carboxylic acid in the present experiment), which is capable of forming a five-membered ring with the Phosphorus center to release the drug, and which nucleophile is only present in O—P5(PEG12)-amidopentyl-Phosphoramidate-N-(L-alanine-tert.-butylester)-O—SN38. Sacituzumab conjugated to O—P5(PEG12)-amidopentyl-Phosphoramidate-N-(4-Methylbenzyl)-O—SN38 is not able to liberate a nucleophile that would be capable of forming a ring structure with the Phosphorus center and is therefore also not active in the in vitro cell viability assay.

### DXD-Based ADCs

Antibody-drug conjugates (ADCs) comprising the anti-Her2 antibody Trastuzumab, a phosphorus(V) moiety according to embodiments of the present invention, and DXD as drug were tested in in vitro potency assays against a Her2-positive cell line and a Her2-negative cell line. The corresponding ADCs with the non-targeted antibody Palivizumab were used as isotype control.

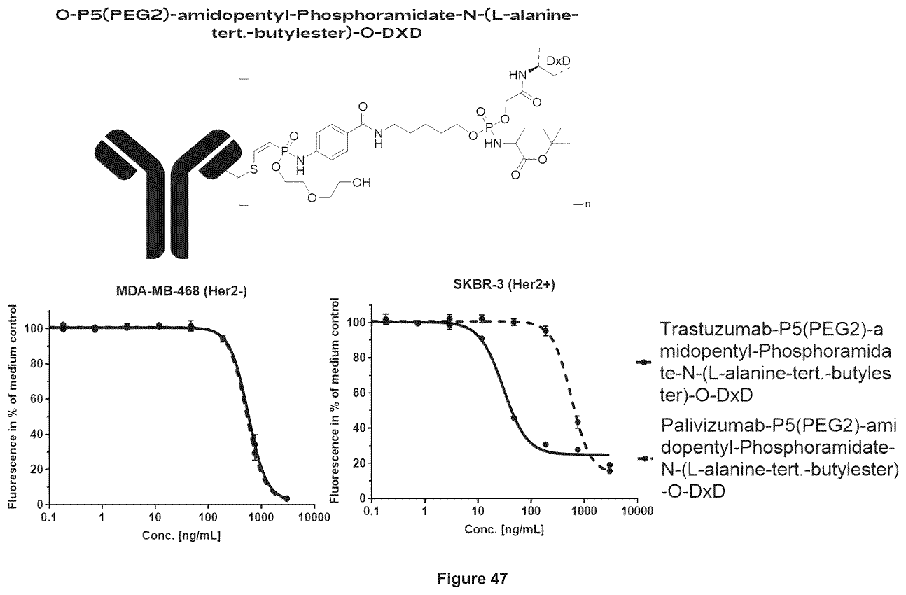

FIG. 47 shows the results of in vitro potency assays of ADCs from compound O—P5(PEG2)-amidopentyl-Phosphoramidate-N-(L-alanine-tert.-butylester)-O-DxD and the antibodies Trastuzumab (anti-Her2, solid) and Palivizumab (isotype, dashed). The in vitro efficacy on a targeted cell line (SKBR-3, Her2+) and a non-targeted cell line (MDAMB-468, Her2−) is depicted.

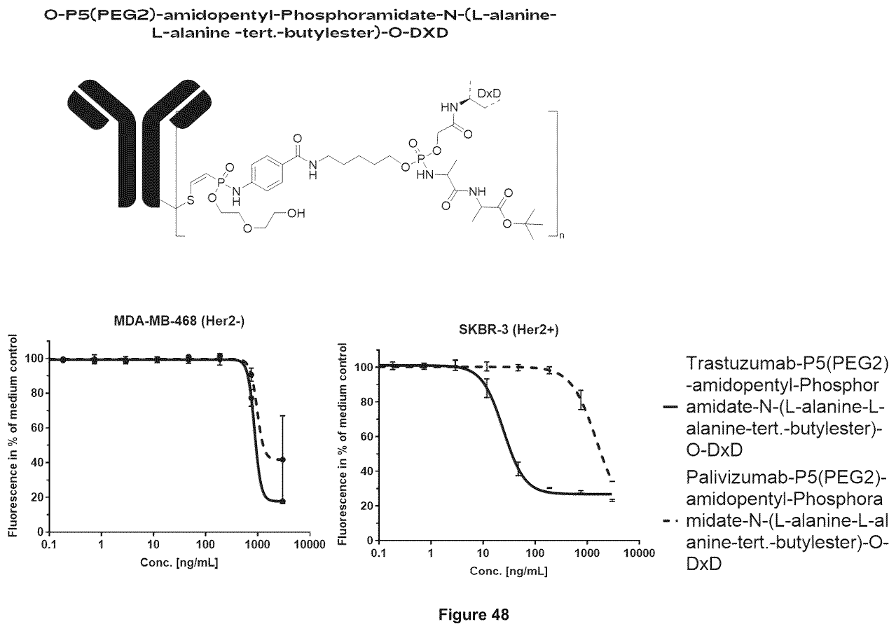

FIG. 48 shows the results of in vitro potency assays of ADCs from compound O—P5(PEG2)-amidopentyl-Phosphoramidate-N-(L-alanine-L-alanine-tert.-butylester)-O-DxD and the antibodies Trastuzumab (anti-Her2, solid) and Palivizumab (isotype, dashed). The in vitro efficacy on a targeted cell line (SKBR-3, Her2+) and a non-targeted cell line (MDAMB-468, Her2−) is depicted.

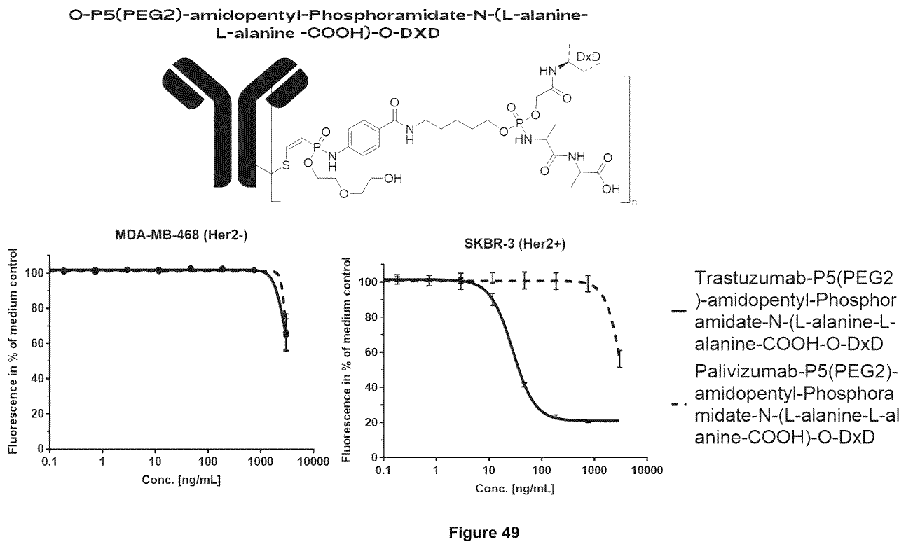

FIG. 49 shows the results of in vitro potency assays of ADCs from compound O—P5(PEG2)-amidopentyl-Phosphoramidate-N-(L-alanine-L-alanine-COOH)—O— DxD and the antibodies Trastuzumab (anti-Her2, solid) and Palivizumab (isotype, dashed). The in vitro efficacy on a targeted cell line (SKBR-3, Her2+) and a non-targeted cell line (MDAMB-468, Her2−) is depicted.
All ADCs comprising a phosphorus(V) moiety in accordance with embodiments of the invention and DXD, which are shown above, exhibit selective cell killing on the targeted cell line, with significantly less effects of the isotype control ADCs.
These experiments demonstrate that the constructs efficiently and selectively deliver the drug (DXD) to the target.

The inventors also compared ADCs from compound O—P5(PEG2)-amidopentyl-Phosphoramidate-N-(L-alanine-L-alanine-COOH)—O-DxD and the anti-Her2-antibody Trastuzumab (in accordance with an embodiment of the invention) with the approved and marketed ADC Enhertu in in vitro potency assays against a Her2-positive and a Her2-negative cell line.

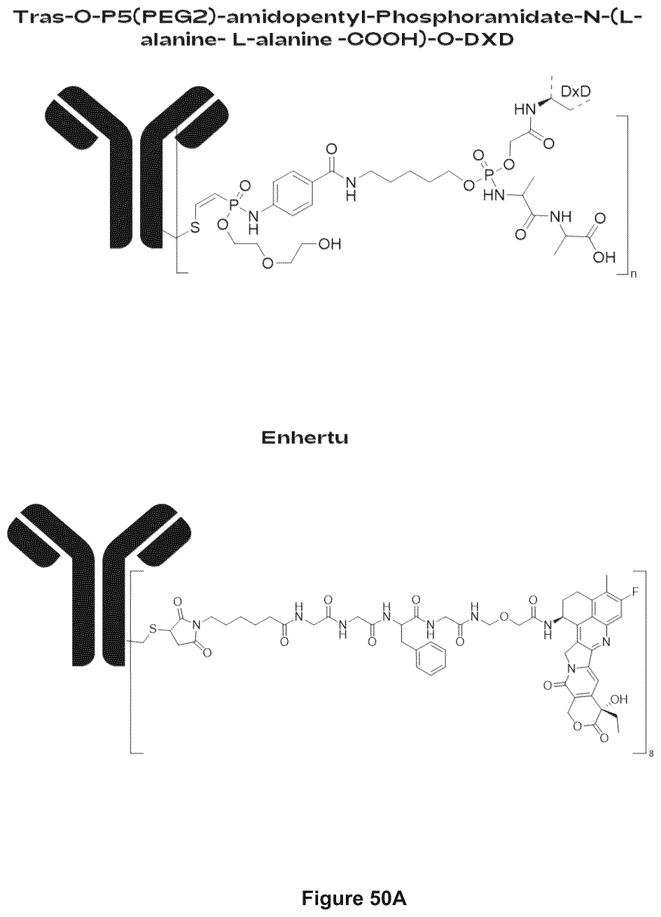

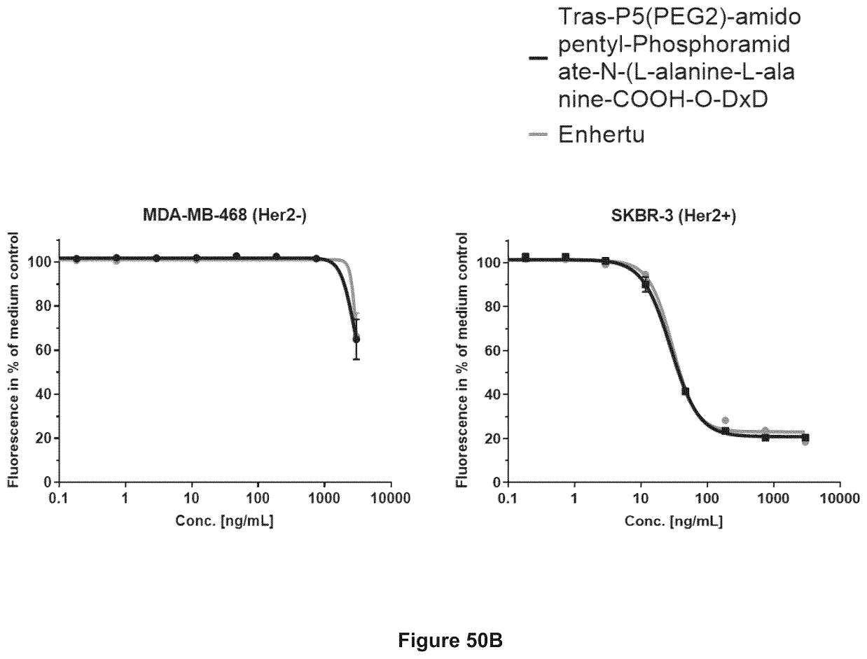

FIGS. 50A and 50B show the results of in vitro potency assays of ADCs from compound O—P5(PEG2)-amidopentyl-Phosphoramidate-N-(L-alanine-L-alanine-COOH)—O-DxD and Trastuzumab (in accordance with an embodiments of the invention, black) and Enhertu (grey). The in vitro efficacy on a targeted cell line (SKBR-3, Her2+) and a non-targeted cell line (MDAMB-468, Her2−) is depicted.
Highly similar cell killing of the ADC comprising a phosphorus(V) moiety (in accordance with an embodiment of the invention) in comparison to the approved and marketed ADC Enhertu is observed, demonstrating efficient and selective delivery of the DXD payload to the target.

### Example 4: Ex Vivo Serum Stability

The inventors also tested the stability in the presence of serum of ADCs from compound O—P5(PEG12)-amidopentyl-Phosphoramidate-N-(L-alanine-tert.-butylester)-O—SN38 conjugated to Sacituzumab (in accordance with an embodiment of the invention) in direct comparison to the approved and marketed ADC Trodelvy.

FIG. 51 shows the result of a direct comparison of ADCs from compound O—P5(PEG12)-amidopentyl-Phosphoramidate-N-(L-alanine-tert.-butylester)-O—SN38 conjugated to Sacituzumab (in accordance with an embodiments of the invention, solid) with Trodelvy (dashed) with regard to the stability in the presence of serum at 37° C. Shown is the drug-to-antibody-ratio over time measured by mass spectrometry (MS) after pull-down from rat serum.
The above stated experiment clearly demonstrates the tremendous increase in stability in the presence of serum, provided by exchanging the CL2A linker of trodelvy with the herein described O—P5(PEG12)-amidopentyl-Phosphoramidate-N-(L-alanine-tert.-butylester).
It is assumed that this increase in stability, which is also confirmed by the selective delivery of the SN38 payload into the targeted cell line, will translate into a more efficacious medication, since more of the active substance (SN38) gets actively delivered to the side of the tumor. Moreover, safer ADCs in the patient are expected in the patient, since premature payload loss in serum circulation leads to off-target toxicities caused by the free drug (SN38 in this case).

The inventors also tested the stability in the presence of serum of ADCs from compound O—P5(PEG2)-amidopentyl-Phosphoramidate-N-(L-alanine-L-alanine-COOH)—O— DxD conjugated to Trastuzumab (in accordance with an embodiment of the invention) in direct comparison to Enhertu.

FIG. 52 shows the result of a direct comparison of ADCs from compound O—P5(PEG2)-amidopentyl-Phosphoramidate-N-(L-alanine-L-alanine-COOH)—O-DxD conjugated to Trastuzumab (in accordance with an embodiment of the invention, solid) with Enhertu (dashed) with regard to the stability in the presence of serum at 37° C. Shown is the drug-to-antibody-ratio over time measured by mass spectrometry (MS) after pull-down from rat serum.
The above stated experiment clearly demonstrates the tremendous increase in stability in the presence of serum, provided by exchanging the Mal-GGFG-DXD linker of Enhertu with the herein described O—P5(PEG2)-amidopentyl-Phosphoramidate-N-(L-alanine-L-alanine-COOH).
It is assumed that this increase in stability, which is also confirmed by the selective delivery of the DXD payload into the targeted cell line, will translate into a more efficacious medication, since more of the active substance (DXD) gets actively delivered to the side of the tumor. Moreover, safer ADCs in the patient are expected in the patient, since premature payload loss in serum circulation leads to off-target toxicities caused by the free drug (DXD in this case).

### Example 5: In Vivo Efficacy of Conjugates

In vivo efficacy of an ADC using a linker system in accordance with embodiments of the invention was evaluated head-to-head to the approved ADC Trodelvy. The same Trop2-targeting antibody Sacituzumab that is applied in Trodelvy has been used, as well as the same alcohol containing SN-38 payload in a drug to antibody ratio (DAR) of 8. The only difference is that the linker system CI2A of Trodelvy was replaced by a linker in accordance with embodiments of the invention (see FIG. 122 for structures).

The results clearly underline the superiority of the linker systems described herein. Increased in vivo activity over marketed Trodelvy is shown in two head-to-head experiments (single dose and double dose). In the double dose experiment, complete tumor remission was observed in almost all treated animals over a long observation period with the linker in accordance with embodiments of the invention, whereas no complete remission was observed in the 5 animals treated with Trodelvy, but only slightly delayed tumor growth compared to the non-treated vehicle control. Moreover, desired targeted selectivity of the linker system described herein was proven with an isotype control antibody, conjugated to eight molecules of the same linker payload system (O—P5(PEG12)-amidopentyl-Phosphoramidate-N-(L-alanine-tert.-butylester)-O—SN38), treated twice. Tumour growth in this group was unaffected and almost identical to the non-treated vehicle control.

FIG. 122 shows a head to head comparison of the in vivo efficacy of the ADC O—P5(PEG12)-amidopentyl-phosphoramidate-N-(L-alanine-tert.-butylester)-O—SN38, which comprises a linker in accordance with embodiments of the invention, with the approved ADC Trodelvy.

All animal experiments were conducted in accordance with German animal welfare law and approved by local authorities. In brief, 1×10⁷ MDA-MB-468 cells (50 μl+50 μl Matrigel) were subcutaneously injected to CB17-Scid mice. Treatment was initiated when tumours reached a tumour volume of about 0.1 cm³ 9 days after implantation. 5 animals per group were either once at day 0 (left in FIG. 122) or twice at day 0 and day 4 (right in

FIG. 122) with 10 mg/kg each treatment day of either Sacituzumab-O—P5(PEG12)-amidopentyl-Phosphoramidate-N-(L-alanine-tert.-butylester)-O—SN38 or Trodelvy and compared to an isotype control. The targeting effect has been confirmed with Palivizumab-O—P5(PEG12)-amidopentyl-Phosphoramidate-N-(L-alanine-tert.-butylester)-O—SN38 (isotype control), that has been dosed at day 0 and day 4 with 10 mg/kg, each dose. Mice were treated via intravenous injection after randomisation into treatment and control groups. Tumour volumes, body weights and general health conditions were recorded throughout the whole study.

### Example 6: In Vitro Efficacy in Dependence of the Length of the Spacer E so that Different Ring Sizes can be Formed Upon Cleavage of the Cleavable Group Z

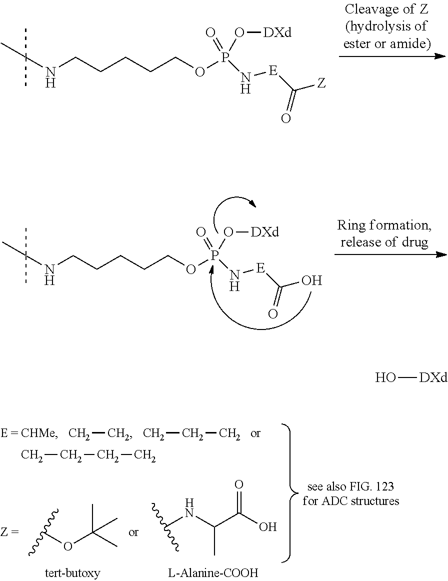

In order to investigate the effectiveness of the release of the drug, the inventors synthesized DXD conjugates that, after cleavage of a cleavable group Z (here: tert-butoxy or L-Alanine-COOH), comprise a moiety W (here: a nucleophilic carboxylic acid group or carboxylate) in different distances to the phosphorous atom to be capable of forming, as described herein, either a five-membered ring (E=CHMe in L-Alanine-L-Alanine-COOH and L-Alanine-tert-butyl ester), a six-membered ring (E=CH₂—CH₂ in β-Alanine-L-Alanine), a seven-membered ring (E=CH₂—CH₂—CH₂ in γ-amino-butyric-acid-L-Alanine) or an eight-membered ring (E=CH₂—CH₂—CH₂—CH₂ in aminovaleric-acid-L-Alanine) together with the phosphorus atom (see above scheme for the proposed mechanism, and FIGS. 123A to 123E for the ADC structures). In vitro results from ADCs made of DXd as a drug and the described linker systems from Trastuzumab (Her2-targeted) and Palivizumab (non-targeted, isotype control) for different Her2-positive cells lines are shown in FIGS. 123A to 123E. It can be seen that all tested ADCs, which have different length of the spacer E so that different ring sizes can be formed together with the phosphorus atom after cleavage of the group Z, are effective for killing the tested cells, with the ADCs comprising anti-Her2-antibody Trastuzumab being more effective than ADCs comprising non-targeted isotype control Palivizumab.

FIGS. 123A to 123E show the in vitro efficacy of ADCs in accordance with embodiments of the invention for cell killing in various cancer cell lines. Tested ADCs have spacers E of different length, so that after cleavage of the group Z different ring sizes can be formed together with the phosphorus atom.

### Example 7: In Vitro Efficacy with Several Hydroxy-Containing Drugs (Aromatic Alcohols)

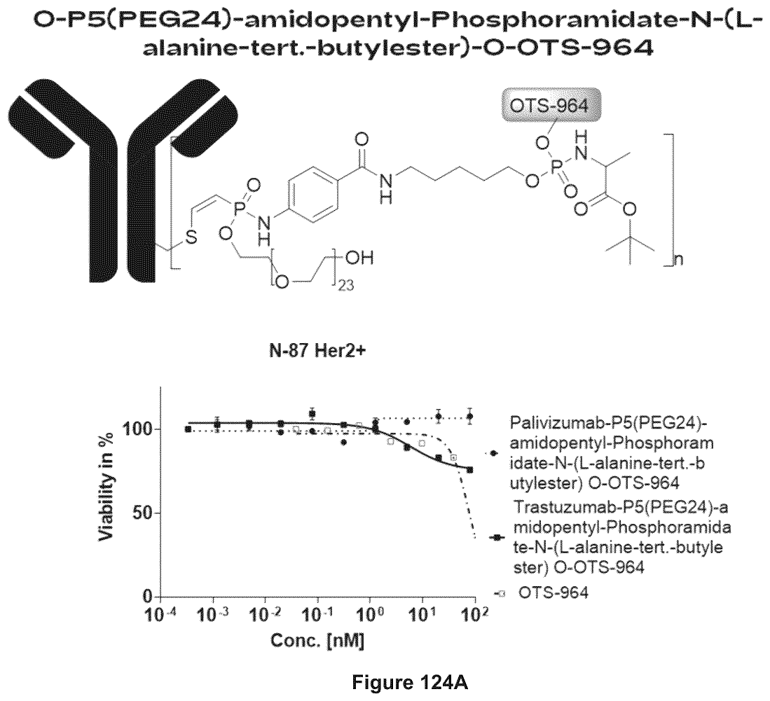

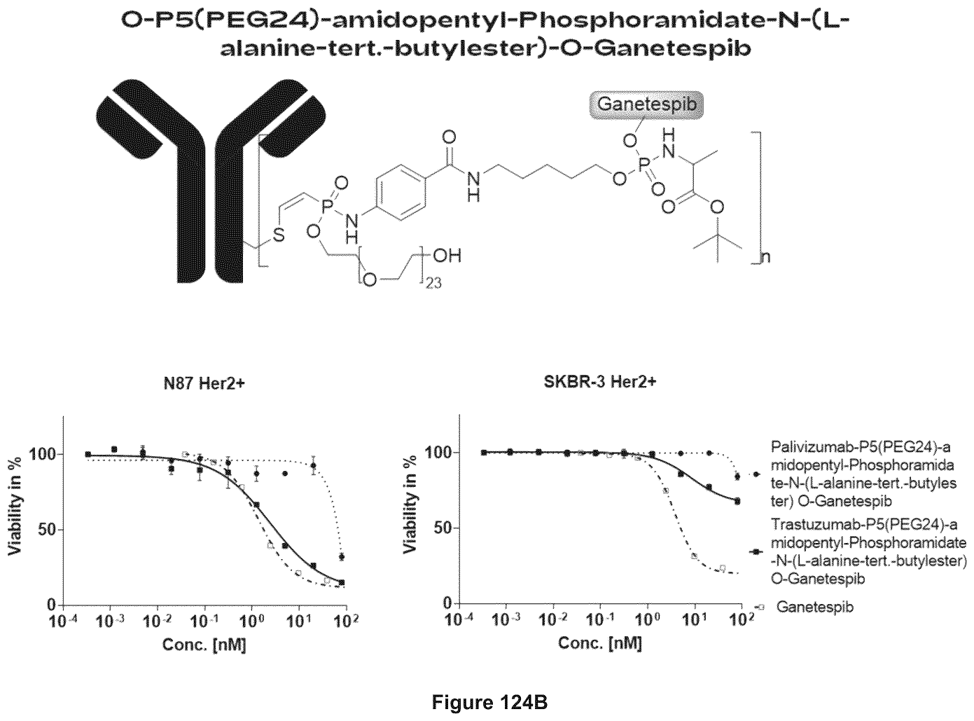

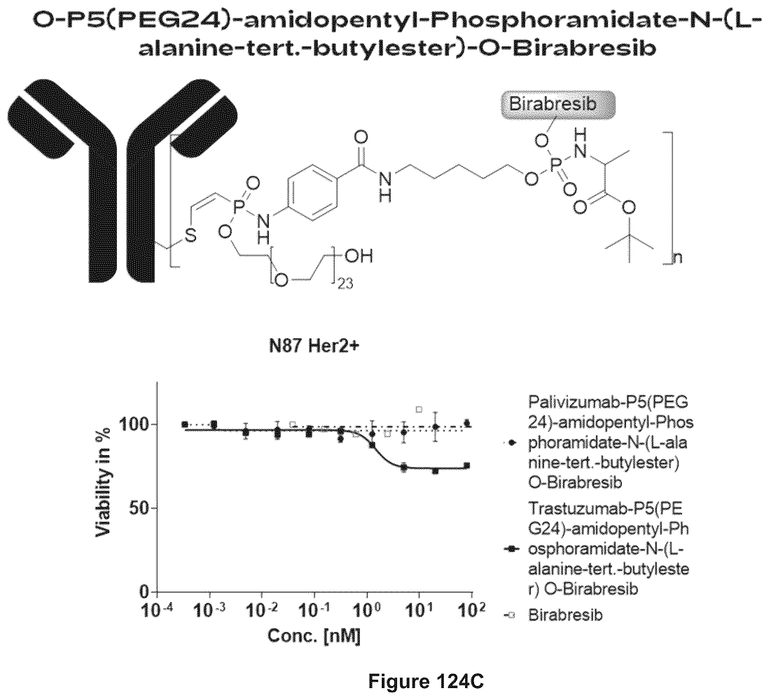

The scope of the linker system of conjugates in accordance with embodiments of the invention has been evaluated with further phenol-(or aromatic alcohol-) containing drugs OTS-964, Ganetespib and Birabresib. OTS-964 is a potent TOPK inhibitor, Ganetespib is a potent CDK inhibitor, and Birabresib is a potent bromodomain inhibitor. The results are shown in FIGS. 124A to 124C. Selective delivery of the drugs into two targeted HER2+ cell lines (N₈₇ and SKBR3) has been demonstrated using ADCs comprising anti-Her2 antibody Trastuzumab, in comparison to the respective isotype control ADC, that carry the same linker payload systems, but the non-targeting antibody palivizumab. Potency of the free payloads (OTS-964, Ganetespib and Birabresib) in the respective cell lines is also shown. Comparison to the Trastuzumab conjugates shows that lower activities of the Trastuzumab conjugates can be directly attributed to lower activities of the unconjugated drugs in the respective cell lines (e.g. OTS-964 and Birabresib) and not due to an insufficient release of the payload. Still, direct comparisons between the targeted Trastuzumab conjugates and the non-targeted Palivizumab conjugates in activity show that OTS-964 and Birabresib get efficiently delivered into the targeted cells via the technology described herein. The drug Ganetespib, which shows the highest activity as free payload, also shows the best in vitro efficacy when conjugated to Trastuzumab via the technology described herein, demonstrating excellent delivery into the targeted cells.

FIGS. 124A to 124C show the in vitro efficacy of ADCs in accordance with embodiments of the invention that comprise aromatic alcohols as drug moieties for cell killing in various cancer cell lines.

### Example 8: In Vitro Efficacy with Several Hydroxy-Containing Drugs (Aliphatic Alcohols)

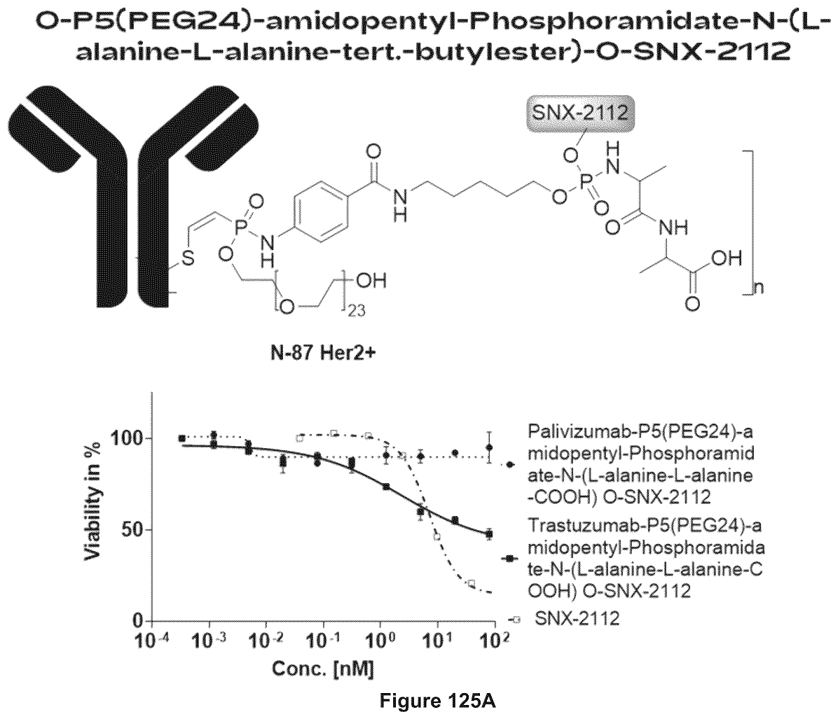

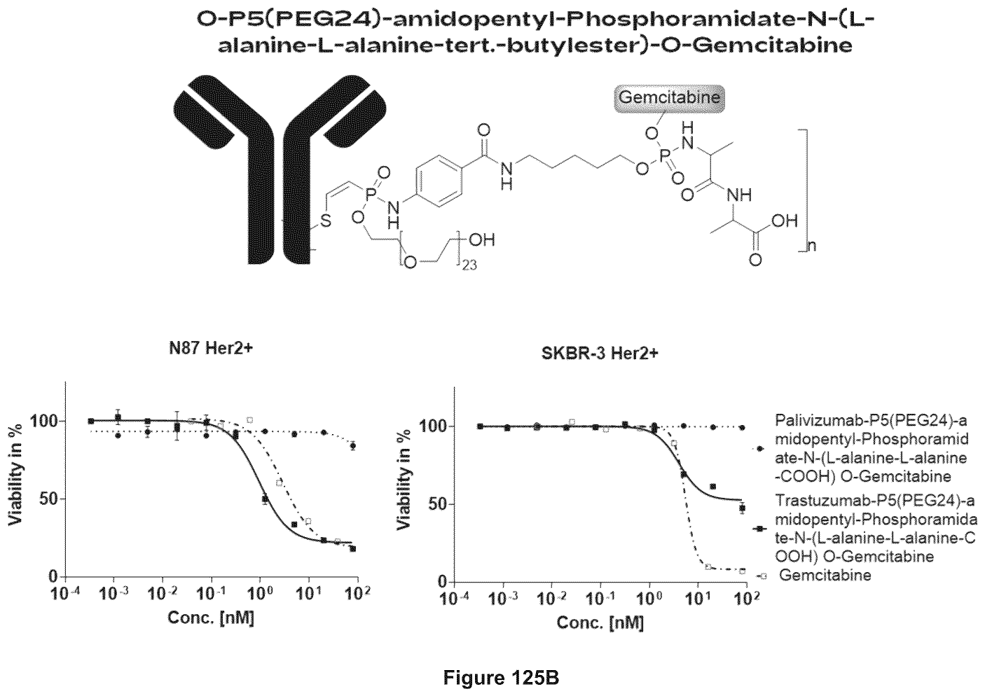

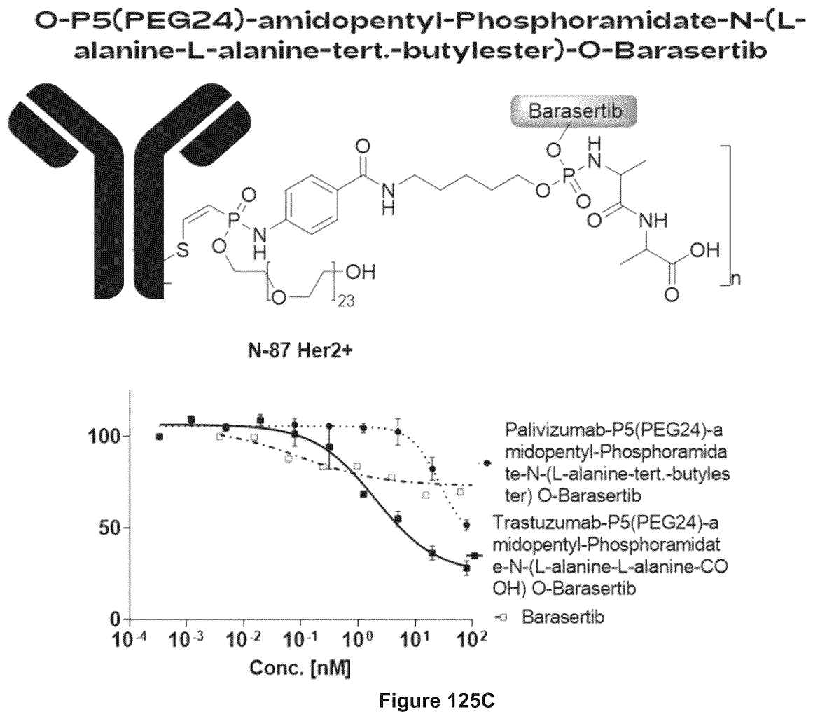

The scope of the linker system described herein has been also evaluated with further aliphatic alcohol-containing drugs SNX-2112, Gemcitabine and Barasertib. SNX-2112 is a potent HSP90-inhibitor, Gemcitabine is a potent ribonucleotide reductase inhibitor, and Barasertib is a potent Aurora B kinase inhibitor. The results are shown in FIGS. 125A to 125C. Selective delivery of the drugs into two targeted HER2+ cell lines (N₈₇ and SKBR3) has been demonstrated using ADCs comprising anti-Her2 antibody Trastuzumab, in comparison to the respective isotype control ADC, that carry the same linker payload systems, but the non targeting antibody palivizumab. Potency of the free payloads (SNX-2112, Gemcitabine and Barasertib) in the respective cell lines is also shown. Direct comparisons between the targeted Trastuzumab conjugates and the non-targeted Palivizumab conjugates in activity show that Barasertib, SNX-2112 and Gemcitabine get efficiently delivered into the targeted cells via the technology described herein.

FIGS. 125A to 125C show the in vitro efficacy of ADCs in accordance with embodiments of the invention that comprise aliphatic alcohols as drug moieties for cell killing in various cancer cell lines.

### Example 9: In Vitro Efficacy of ADC Variants Comprising Different Combinations of Chemical Groups

### SN38 Based ADCs

9A. In Vitro Efficacy of ADCs Comprising Different Spacers E and/or Cleavable Groups Z

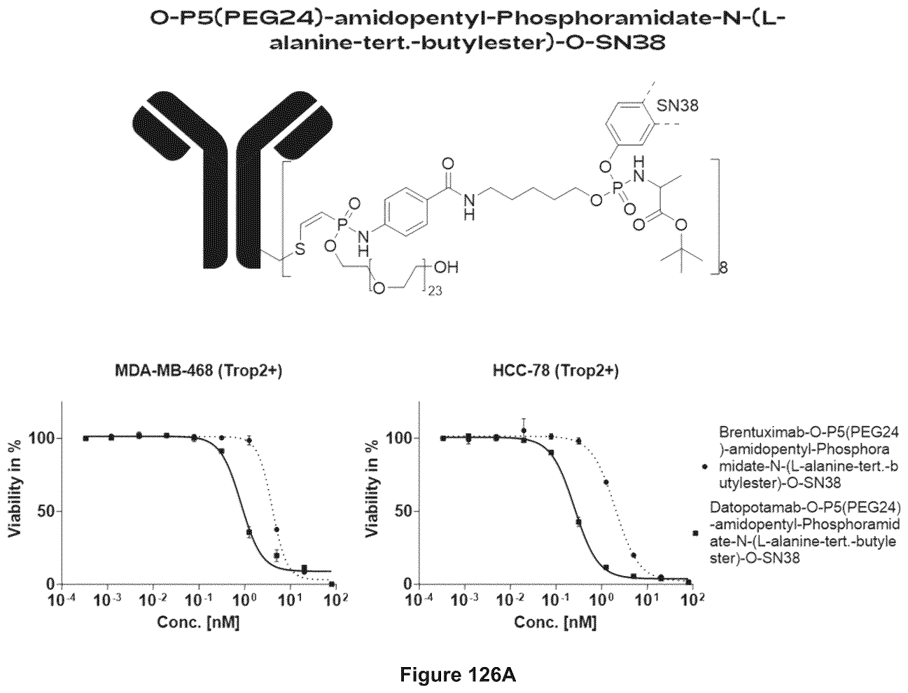

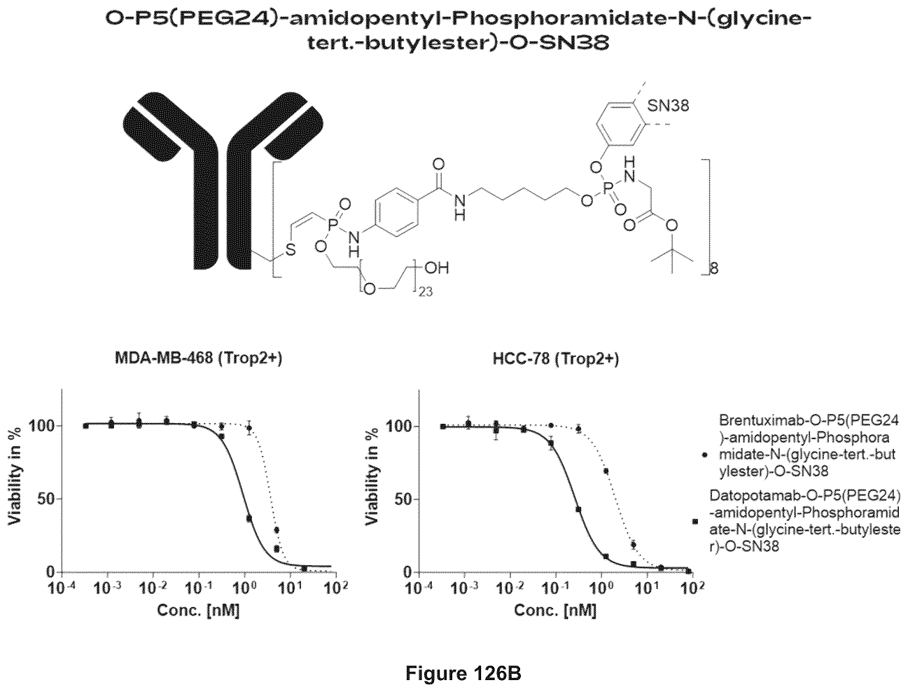

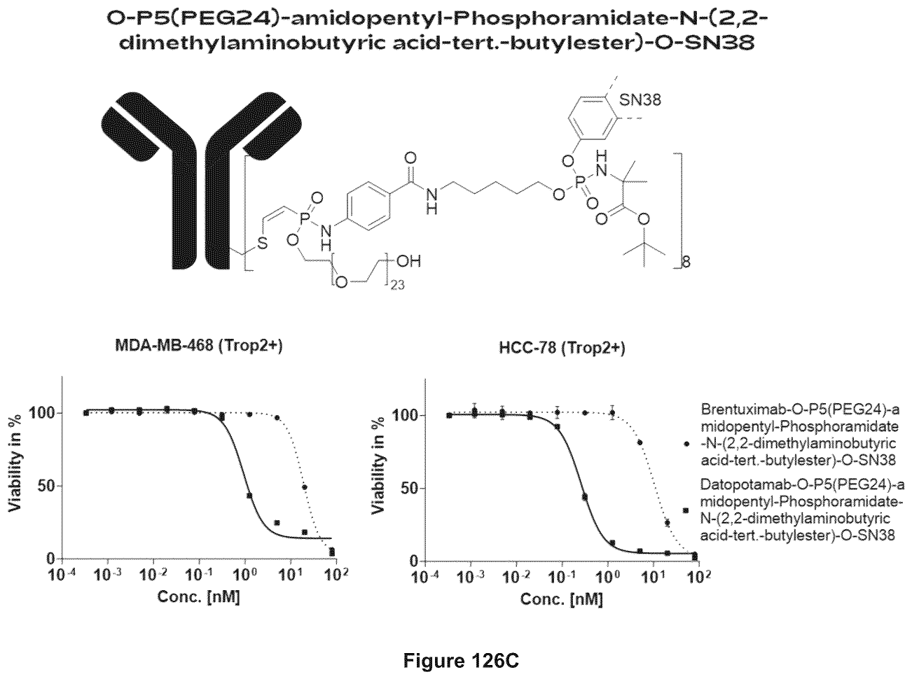

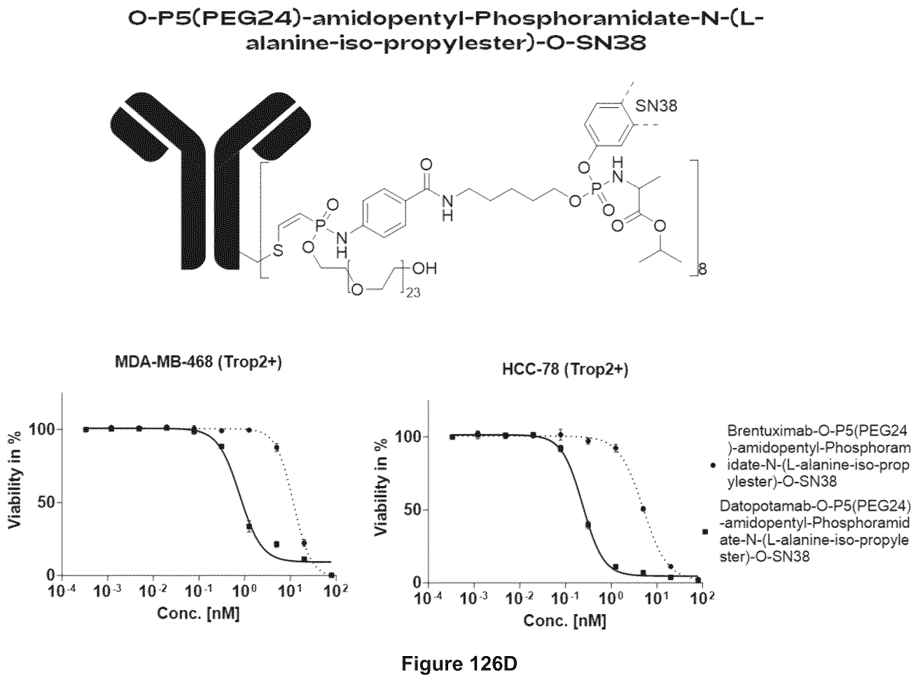

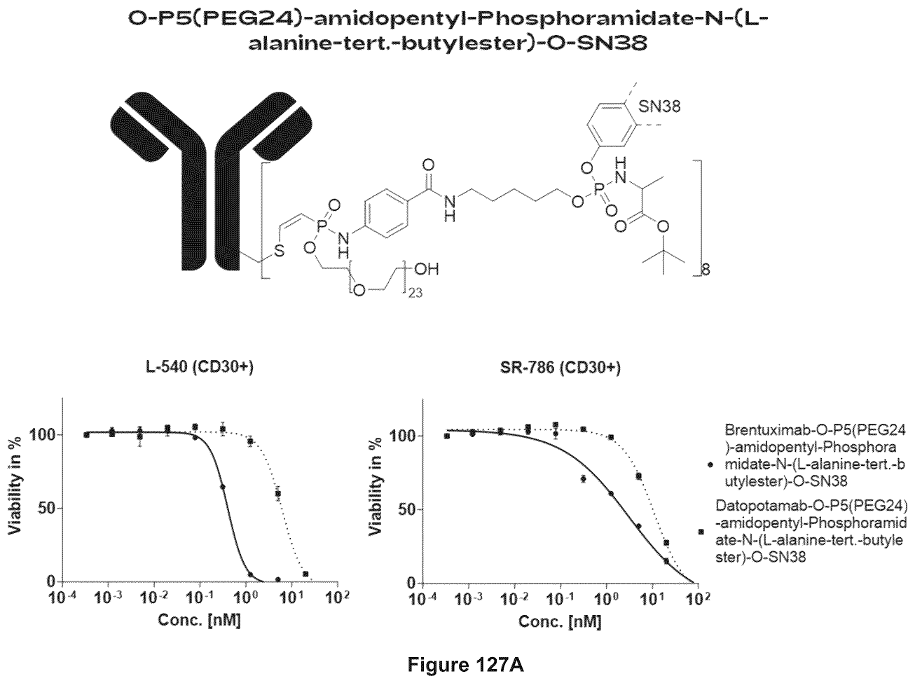

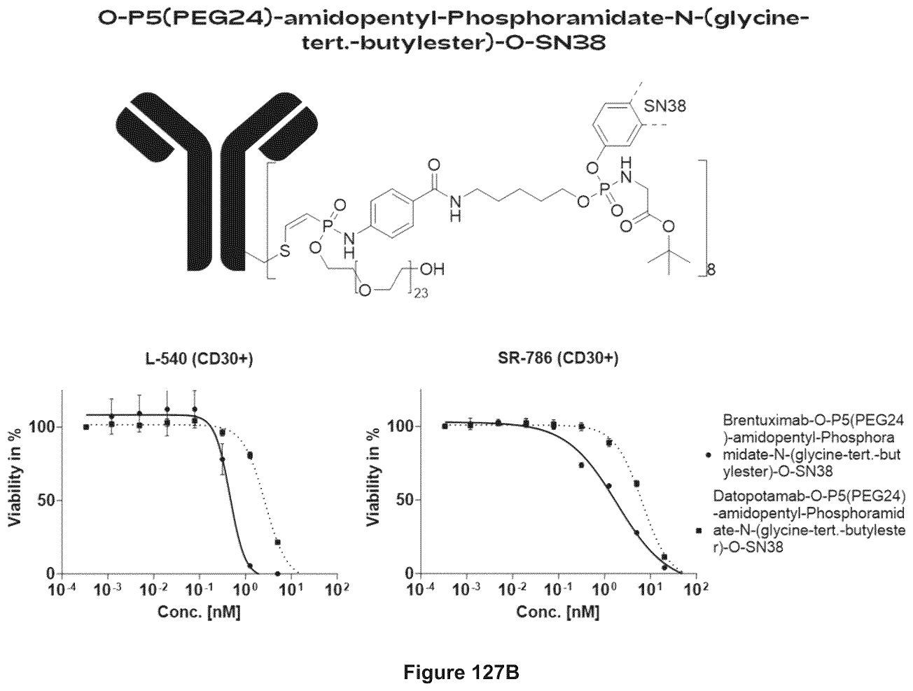

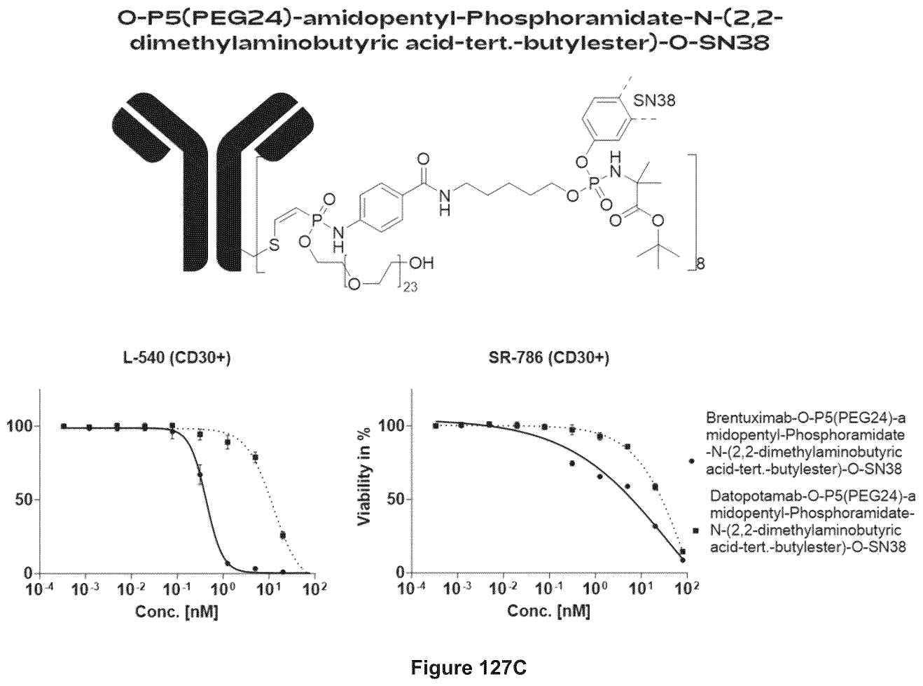

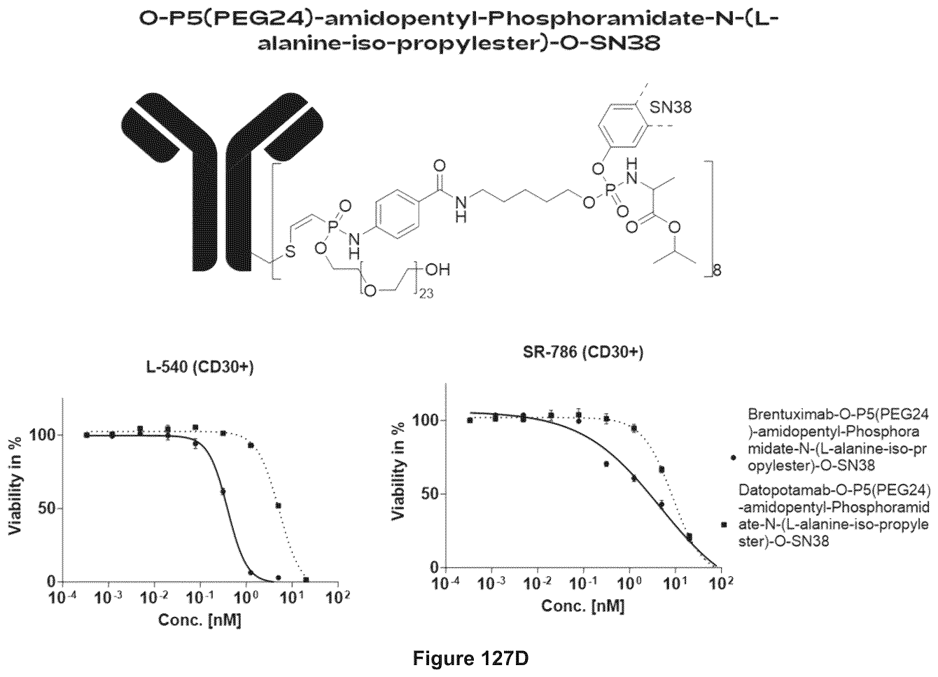

In order to investigate the effectiveness of the release of the drug, the inventors synthesized SN38 conjugates comprising different combinations of spacer E and cleavable group Z. Tested ADC variants comprised as spacer E either Glycine (E=CH₂) or 2,2-dimethylaminobutyric acid (E=CMe₂), and as cleavable group Z isopropylester (Z=iPr). An alternative tested ADC variant comprised alanine as spacer E and tert-butyl ester as cleavable group Z (E=CHMe, Z=tBu). The results of ADCs in accordance with embodiments of the invention comprising SN38 as drug and the described linker systems from Datopotamab (Trop2-targeted) and Brentuximab (non-targeted isotype control) against the two different Trop2-positive cell lines (MDA-MB-468) and (HCC-78) are shown in FIGS. 126A to 126D. The results of ADCs in accordance with embodiments of the invention comprising SN38 as drug and the described linker systems from Brentuximab (CD30-targeted) and Datopotamab (non-targeted isotype control) against the two different CD30-positive cell lines (L-540) and (SR-786) are shown in FIGS. 127A to 127D. These results clearly demonstrate selective target mediated delivery by the conjugated antibody and effective release of the drug, independently of the RBM that is used. Moreover, these results show that numerous different chemical groups can be used as spacer E and cleavable group Z, respectively, and that these different chemical entities can be freely combined without negatively affecting activity of the conjugates.

9B. In Vitro Efficacy of ADCs Comprising a Cyclic Group in Linker L in Combination with Different Spacers E and/or Cleavable Groups Z

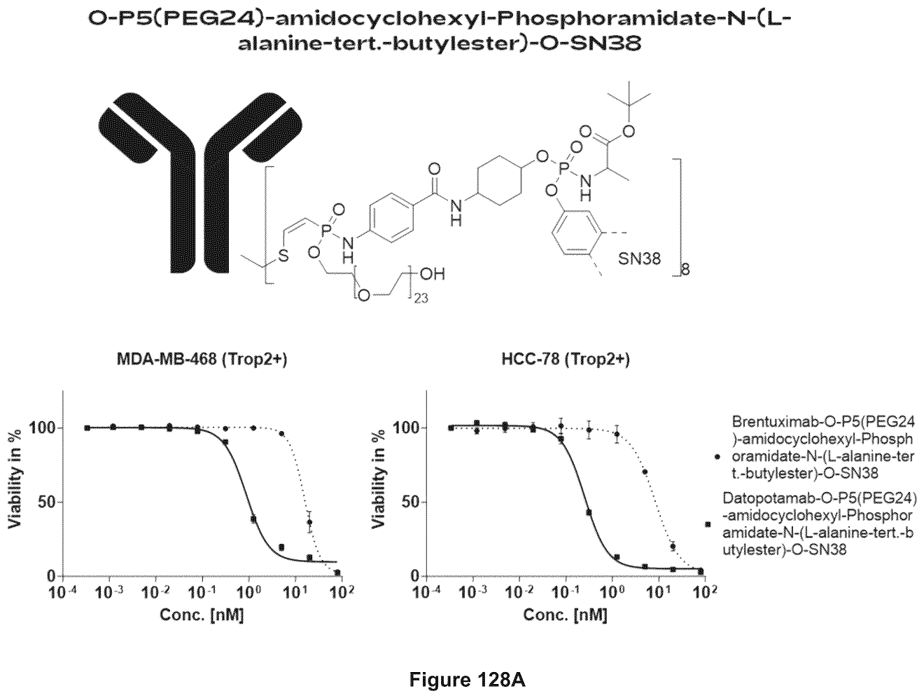

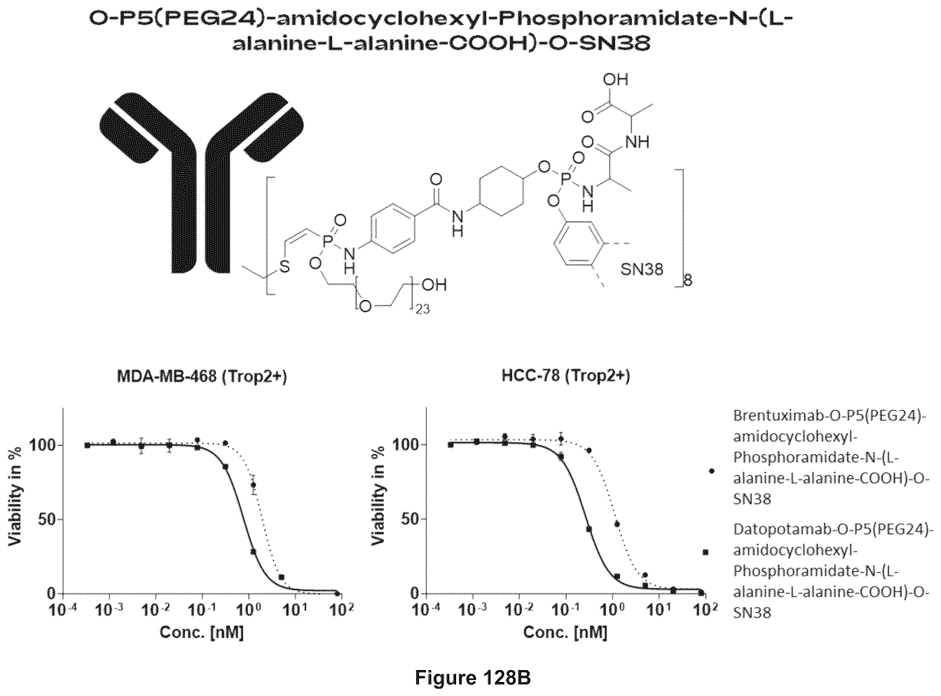

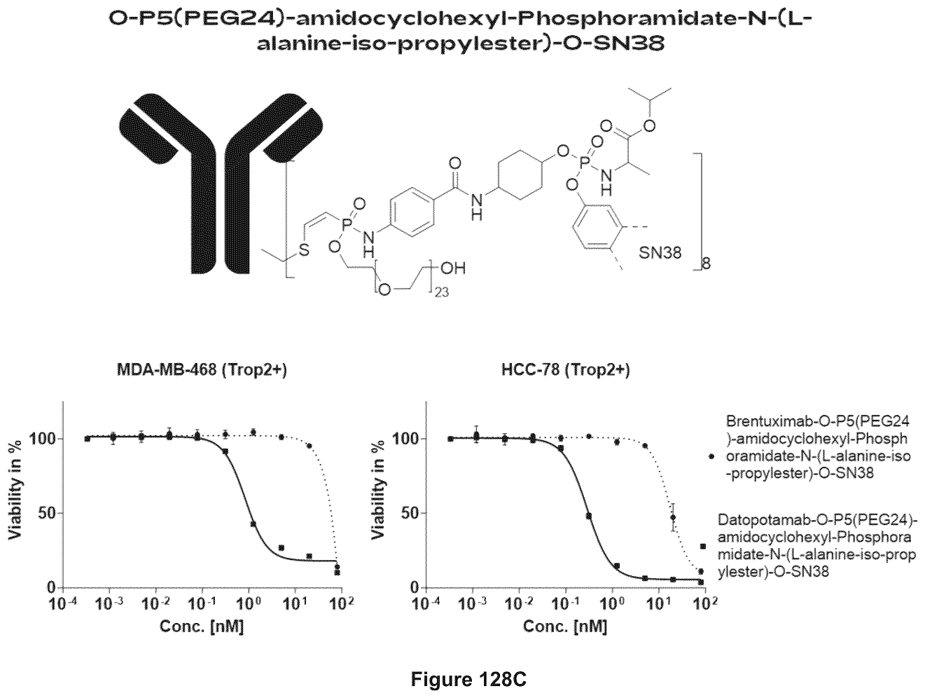

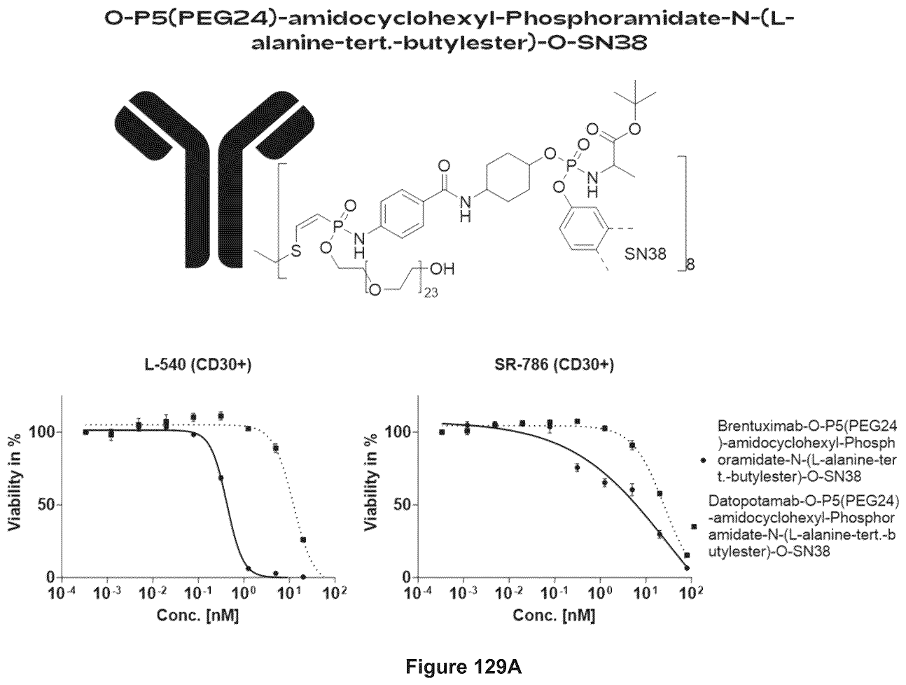

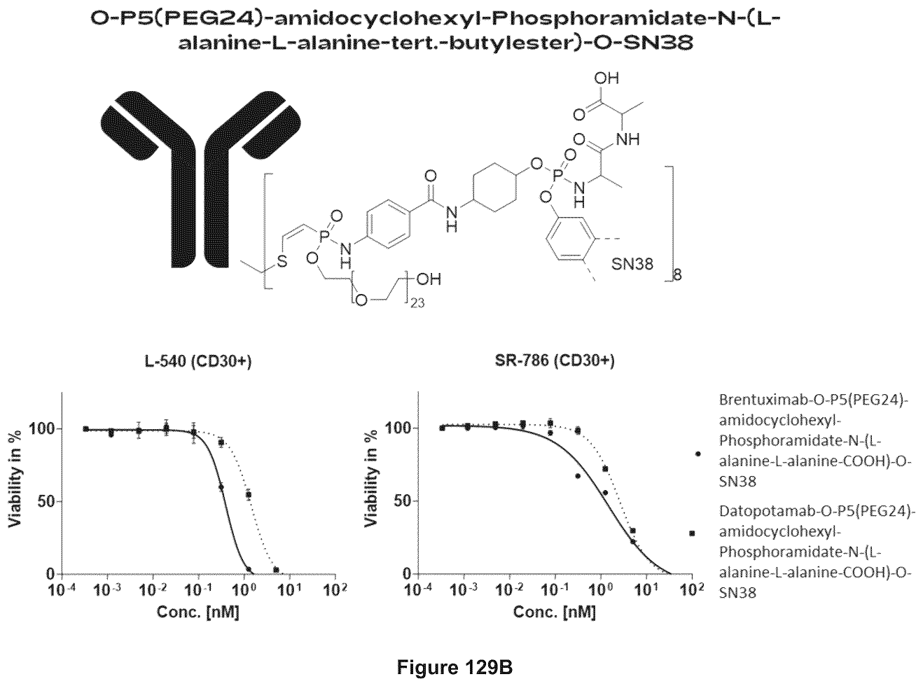

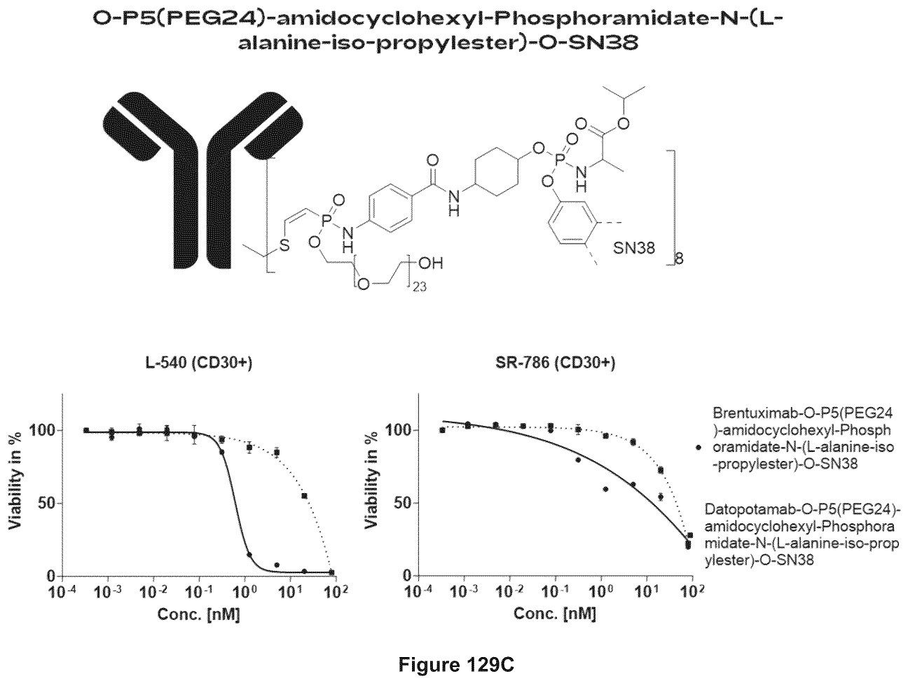

In order to investigate the effectiveness of the release of the drug, the inventors synthesized SN38 conjugates comprising the cyclic group cyclohexyl in linker L in combination with varying combinations for spacer E and cleavable group Z, respectively. In particular, ADC variants comprising cyclohexyl as an embodiment of group Q in linker L with the following combinations of spacer E and cleavable group Z were tested: (i) E=CHMe and Z=tBu); (ii) E=CHMe and Z=—NH—CHMe-COOH; and (iii) E=CHMe and Z=−iPr. The results of ADCs in accordance with embodiments of the invention comprising SN38 as drug and the described linker systems from Datopotamab (Trop2-targeted) and Brentuximab (non-targeted isotype control) against the two different Trop2-positive cell lines (MDA-MB-468) and (HCC-78) are shown in FIGS. 128A to 128C. The results of ADCs in accordance with embodiments of the invention comprising SN38 as drug and the described linker systems from Brentuximab (CD30-targeted) and Datopotamab (non-targeted isotype control) against the two different CD30-positive cell lines (L-540) and (SR-786) are shown in FIGS. 129A to 129C. These results clearly demonstrate selective target mediated delivery by the conjugated antibody and effective release of the drug independently of the RBM that is used. More importantly, these results underline the high versatility of system, i.e. varying combinations of groups of different chemical nature in accordance with the invention can be used for the linker L, spacer E and cleavable group Z without negatively affecting activity of the conjugates.

9C. In Vitro Efficacy of ADCs Comprising Variations in Spacer E, Moiety W and/or Cleavable Group Z

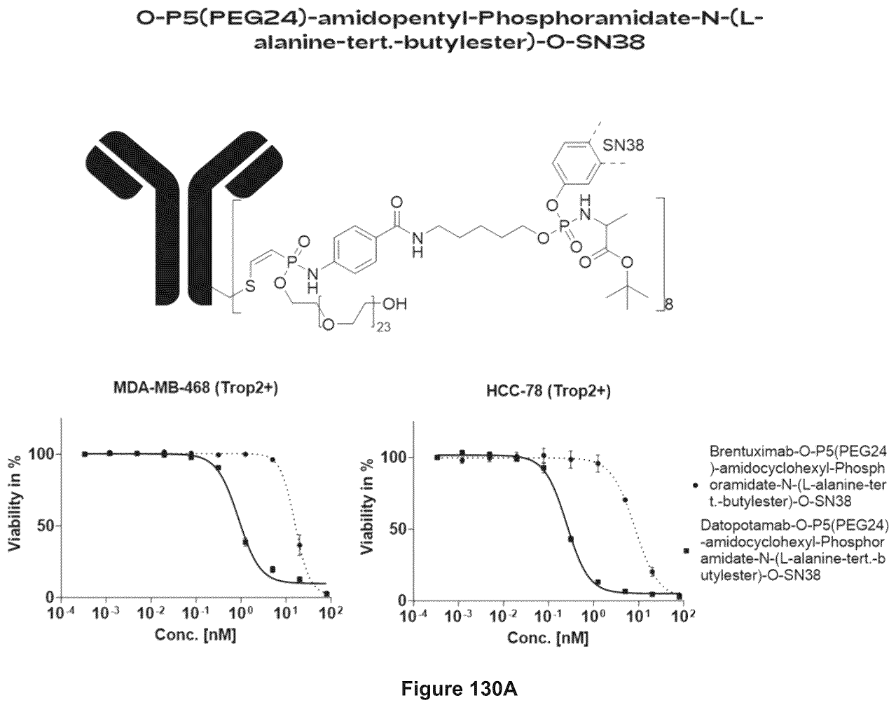

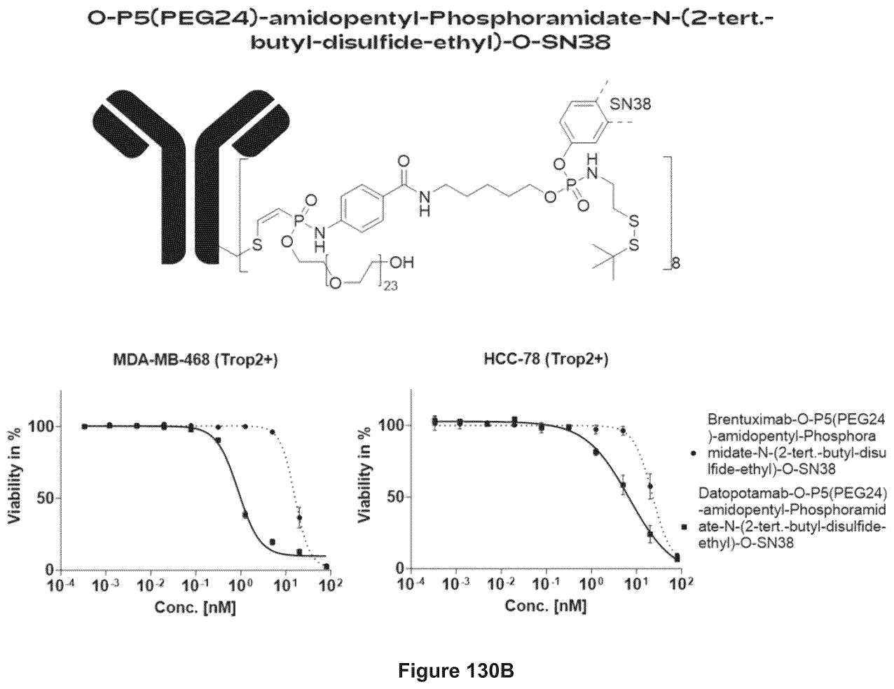

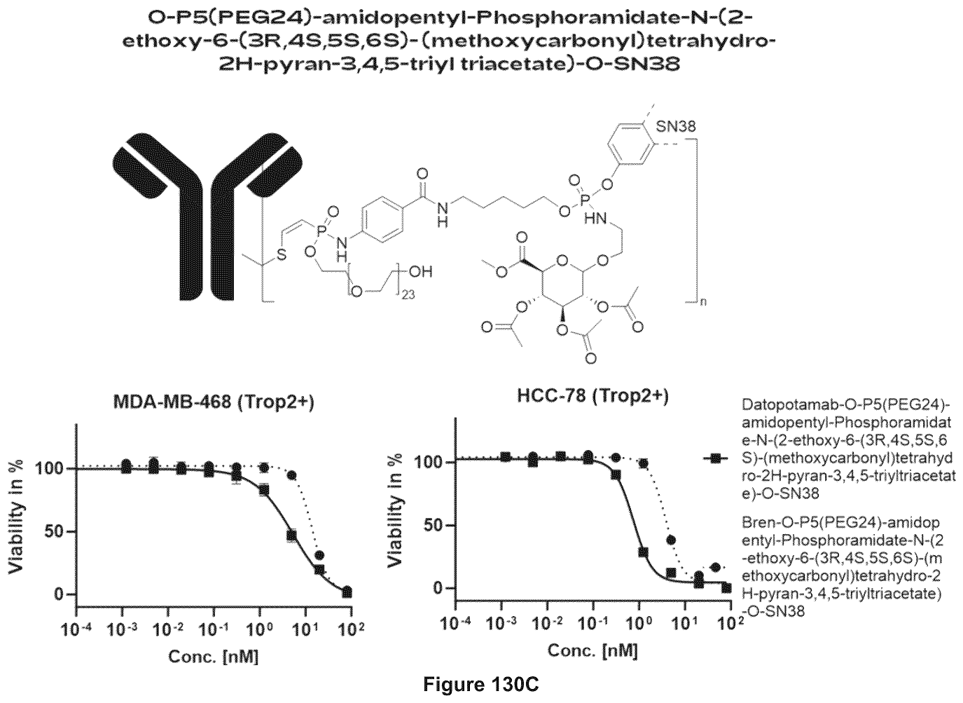

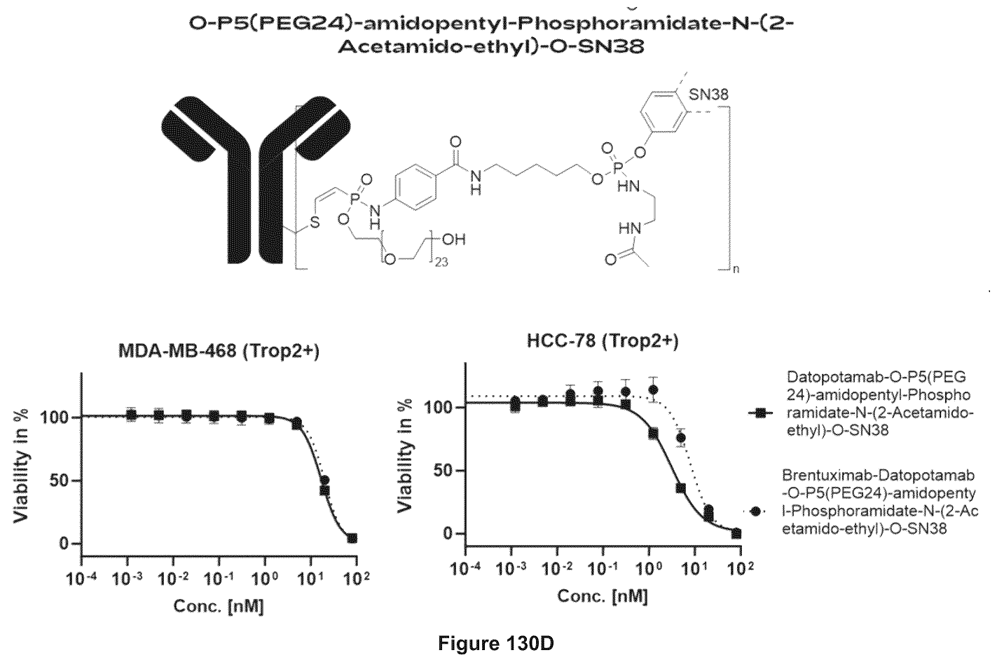

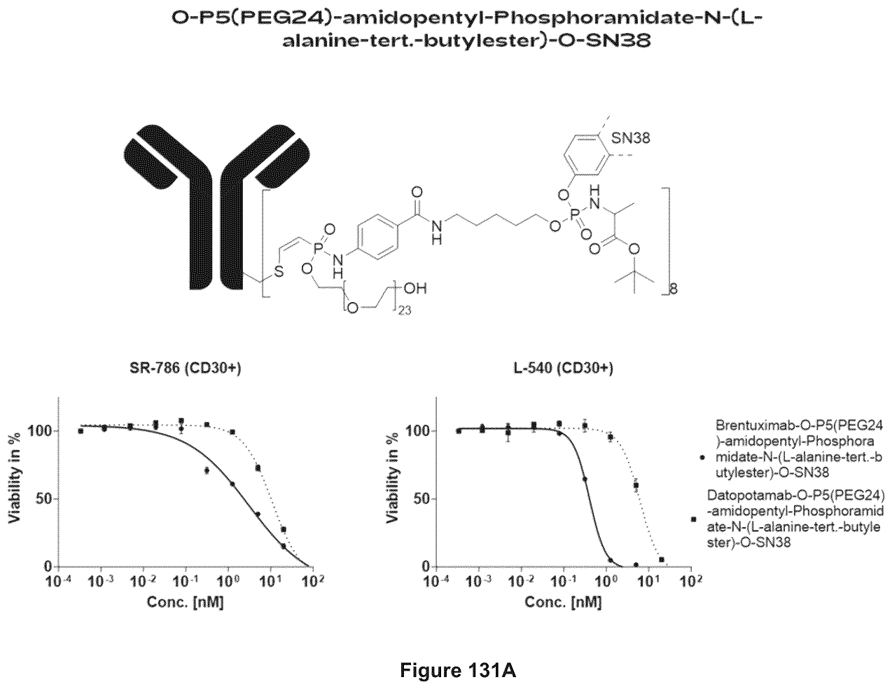

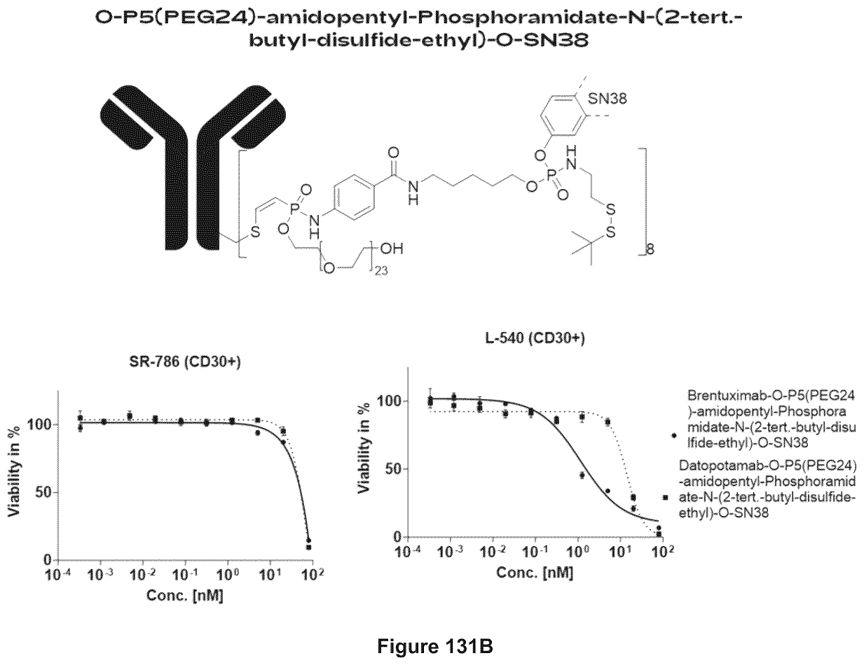

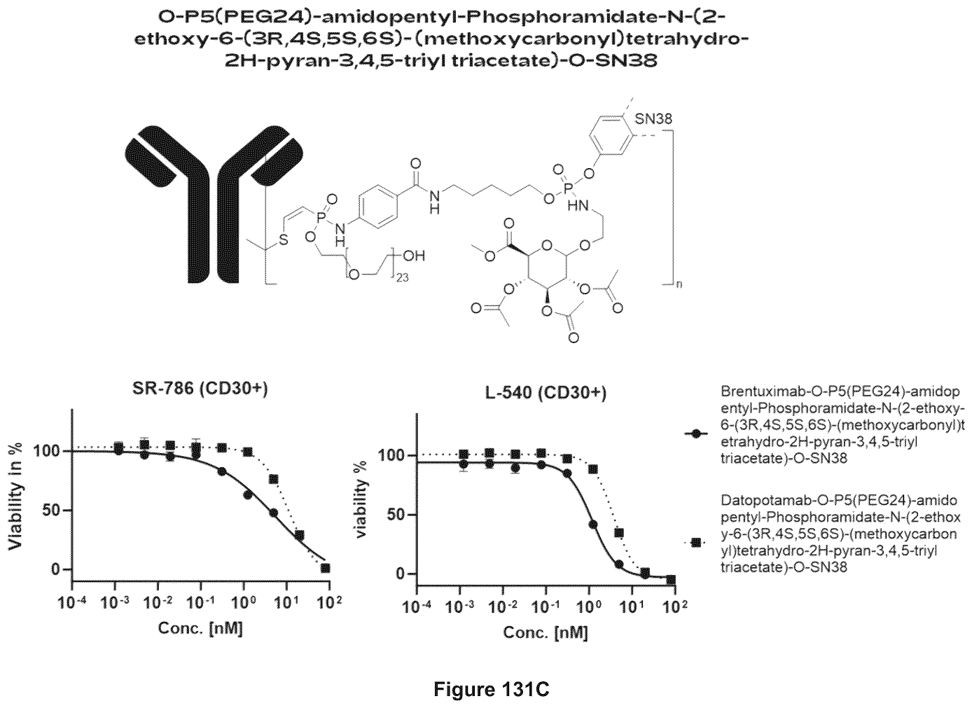

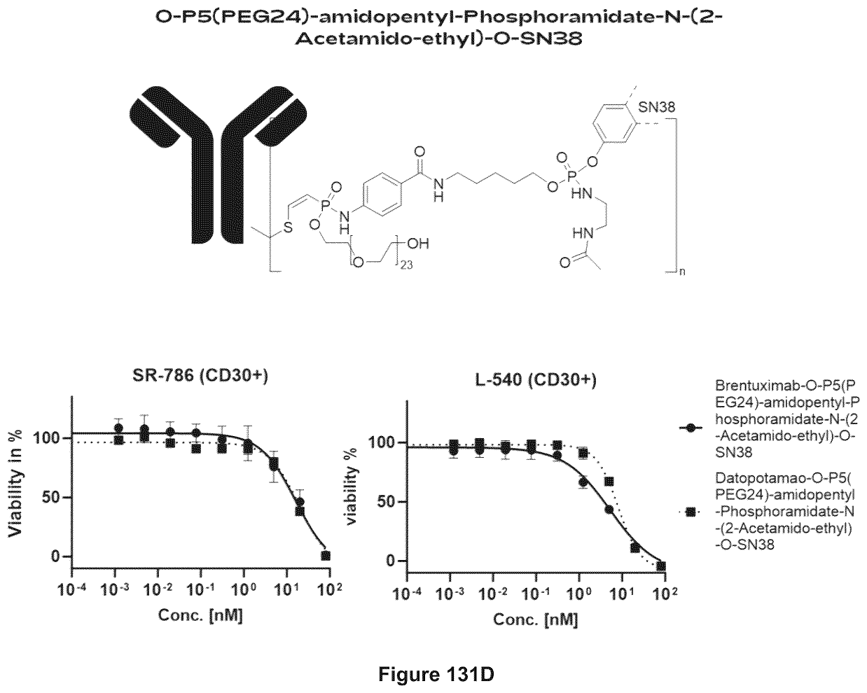

In order to demonstrate the broad spectrum of new improved ways of effective and targeted drug release, the inventors synthesized SN38 conjugates comprising variations in spacer E, moiety W and/or cleavable group Z. In particular, the ADCs were designed so that they liberated a carboxylic acid or a derivative thereof, a thiol, a hydroxy-function or an amine after cleavage of the respective cleavable group Z. Synthesized and tested ADC variants liberating the described nucleophiles after cleavage of the bond between groups W and Z are depicted in FIGS. 130A to 130D and FIGS. 131A to 131D. The results of ADCs in accordance with the described embodiments of the invention comprising SN38 as drug and the described linker systems from Datopotamab (Trop2-targeted) and Brentuximab (non-targeted isotype control) against the two different Trop2-positive cell lines (MDA-MB-468) and (HCC-78) are shown in the FIGS. 130A to 130D. The results of ADCs in accordance with the described embodiments of the invention comprising SN38 as drug and the described linker systems from Brentuximab (CD30-targeted) and Datopotamab (non-targeted isotype control) against the two different CD30-positive cell lines (L-540) and (SR-786) are shown in the FIGS. 131A to 131D. Thus, these results clearly demonstrate that the chemical nature of the sidechain at the P described as Y¹-E-W-Z is not particularly limited and can be widely varied. Moreover, it again highlights the versatility of system in that it can be adapted to be susceptible to completely different ways of cleavage. As, for example, demonstrated, disulfide and glucuronidase cleavable moieties function to liberate the drug inside of the targeted cell. Moreover, it shows that many different moieties W are capable of forming a ring together with the spacer E, Y1 and P after liberation (i.e. cleavage of Z). In particular, these results demonstrate that, for example, a liberated carboxylic acid moiety as well as a liberated thiol or a liberated amine or alcohol is capable of forming a 3-8-membered ring thereby facilitating traceless release of the drug attached via X to the phosphorus atom, when delivered into the targeted cell via a receptor binding molecule RBM.

### 9D. In Vitro Efficacy of ADCs Comprising SN38 or Exatecan as Drug and Different Combinations of Groups X, Y¹ and M

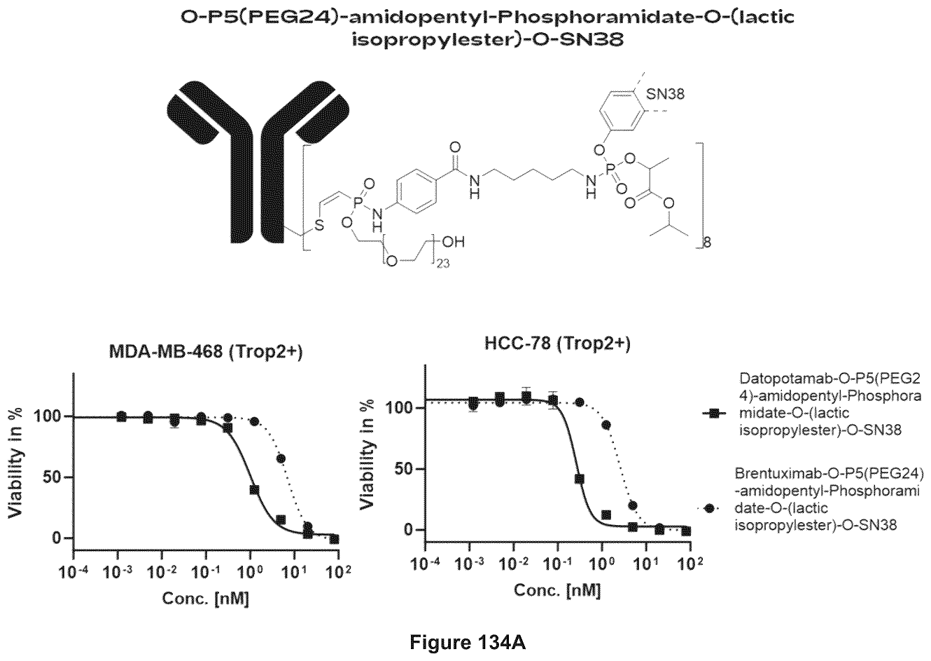

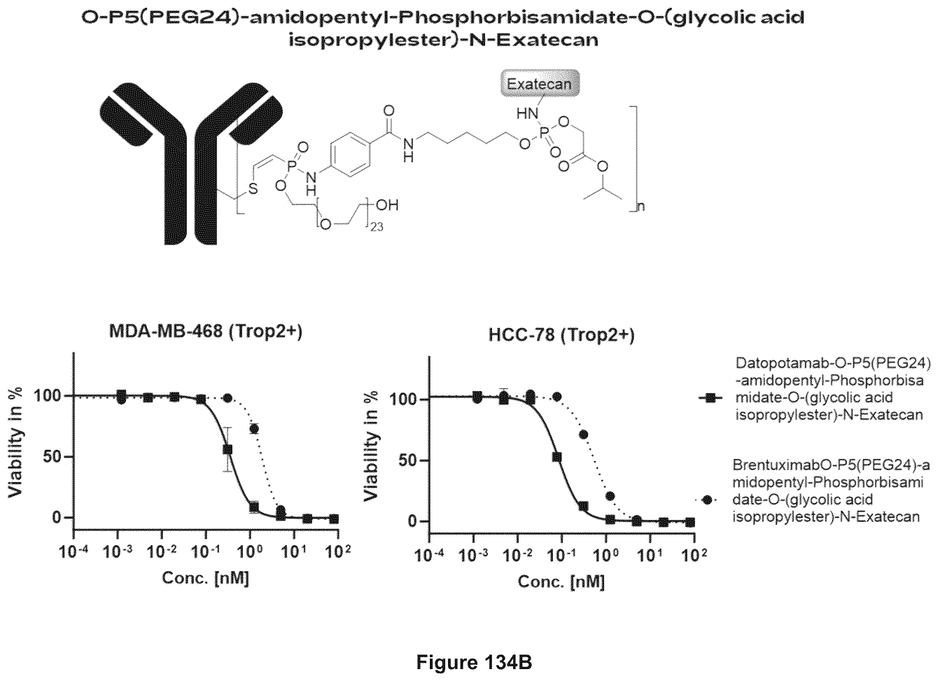

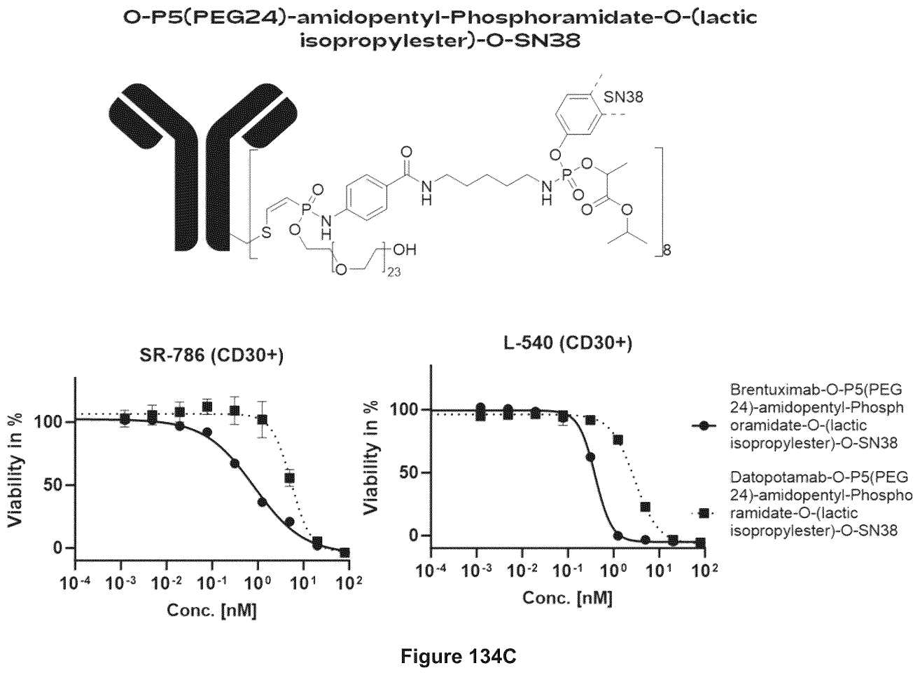

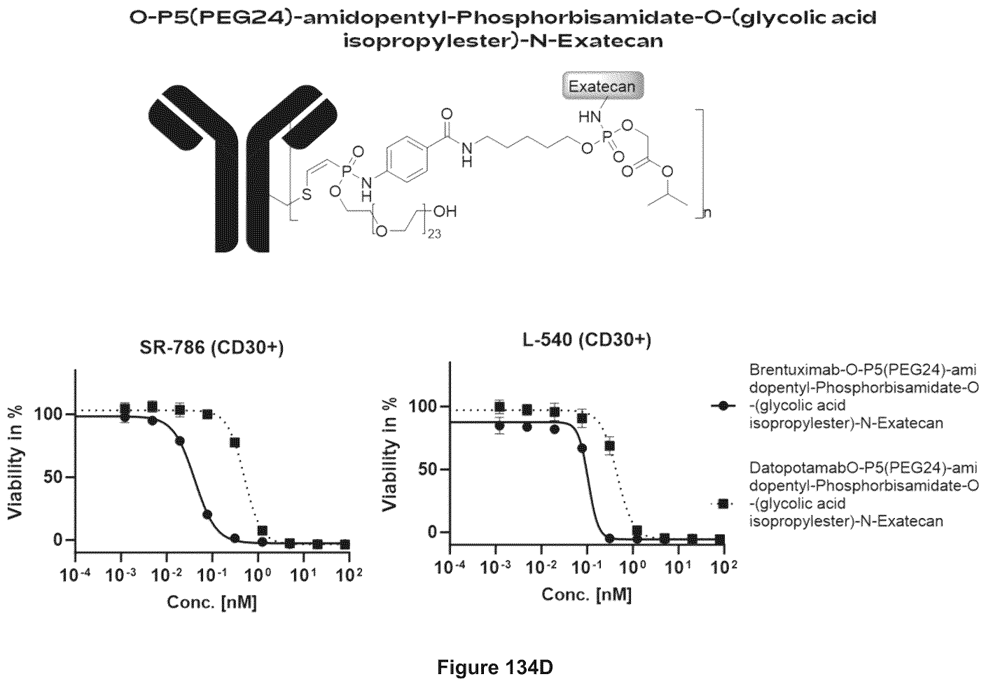

In order to demonstrate variability of the substitution pattern of the central phosphorus P, the inventors synthesized a SN38 conjugate comprising NH as moiety M and O as moiety Y¹ and X, and an Exatecan conjugate comprising O as moiety M as well as Y¹ and NH as moiety X. The structures of the tested ADC variants are depicted in FIGS. 134A and 134B. Effectiveness of the release of the drugs was investigated with these ADCs in accordance with embodiments of the invention comprising SN38 or Exatecan as drug and the described linker systems from Datopotamab (Trop2-targeted) and Brentuximab (non-targeted isotype control) and the results against the two different Trop2-positive cell lines (MDA-MB-468) and (HCC-78) are shown. The results of ADCs in accordance with the described embodiments of the invention comprising SN38 and Exatecan as drug and the described linker systems from Datopotamab (Trop2-targeted) and Brentuximab (non-targeted isotype control) against the two different Trop2-positive cell lines (MDA-MB-468) and (HCC-78) are shown in the FIGS. 134A and 134B. The results of ADCs in accordance with the described embodiments of the invention comprising SN38 as drug and the described linker systems from Brentuximab (CD30-targeted) and Datopotamab (non-targeted isotype control) against the two different CD30-positive cell lines (L-540) and (SR-786) are shown in the FIGS. 134C and 134D. These results clearly demonstrate selective target mediated delivery by the conjugated antibody and effective release of the drug, independently of the drugs' attachment atom X to the P-atom. More importantly, these results underline the high versatility of system, i.e. varying combinations of groups of different chemical nature in accordance with the invention can be used for the moiety M, Y¹ and X without negatively affecting activity of the conjugates.

### DXD Based ADCs

9E. In Vitro Efficacy of ADCs Comprising Variations in Spacer E, Moiety W and/or Cleavable Group Z

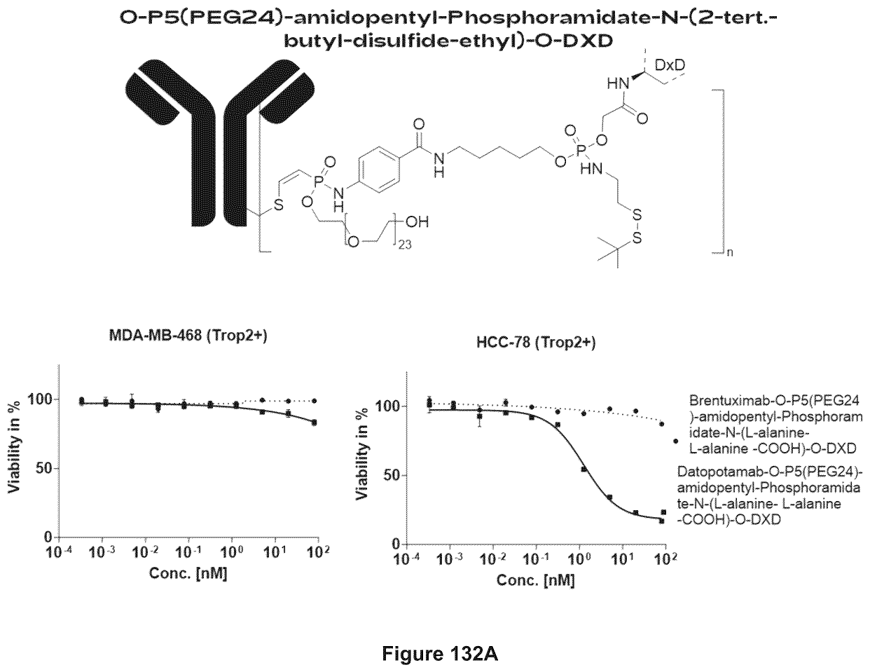

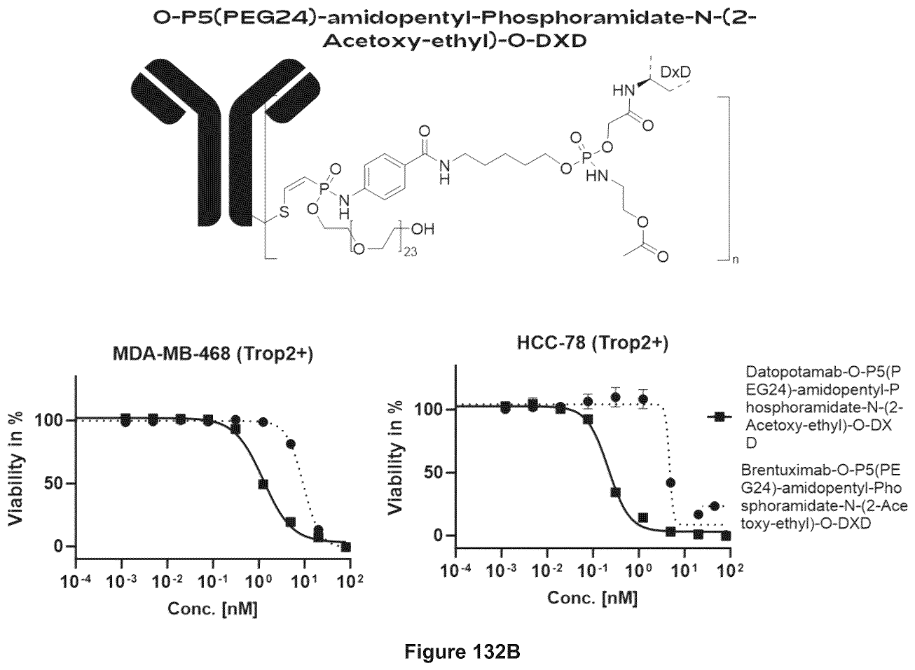

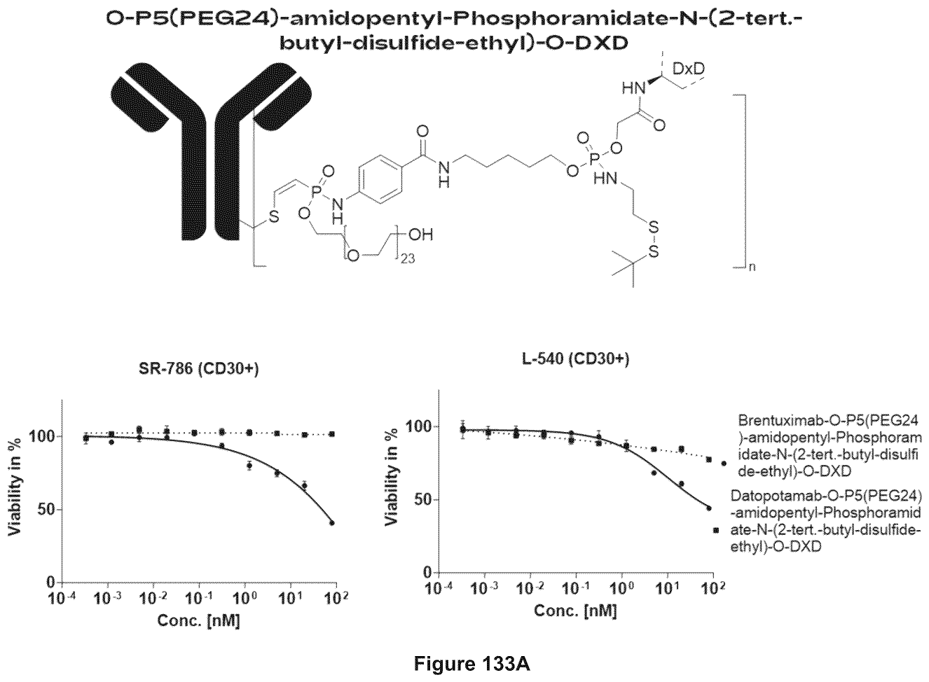

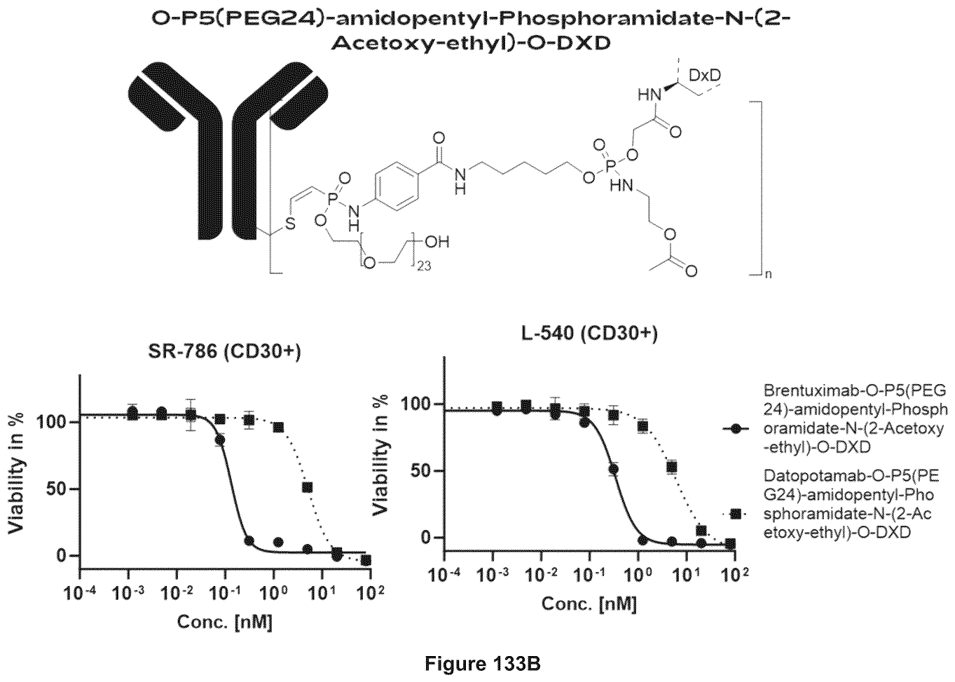

In order to demonstrate the broad spectrum of new improved ways of effective and targeted drug release, the inventors synthesized Dxd conjugates comprising variations in spacer E, moiety W and/or cleavable group Z. In particular, the ADCs were designed so that they liberated a thiol or a hydroxy-function after cleavage of the respective cleavable group Z. Synthesized and tested ADC variants liberating the described nucleophiles after cleavage of the bond between groups W and Z are depicted in FIGS. 132A and 132B and FIGS. 133A and 133B. The results of ADCs in accordance with the described embodiments of the invention comprising SN38 as drug and the described linker systems from Datopotamab (Trop2-targeted) and Brentuximab (non-targeted isotype control) against the two different Trop2-positive cell lines (MDA-MB-468) and (HCC-78) are shown in the FIGS. 132A and 132B. The results of ADCs in accordance with the described embodiments of the invention comprising SN38 as drug and the described linker systems from Brentuximab (CD30-targeted) and Datopotamab (non-targeted isotype control) against the two different CD30-positive cell lines (L-540) and (SR-786) are shown in the FIGS. 133A and 133B. Thus, these results clearly demonstrate that the chemical nature of the sidechain at the P described as Y¹-E-W-Z is not particularly limited and can be widely varied. Moreover, it again highlights the versatility of system in that it can be adapted to be susceptible to completely different ways of cleavage. As, for example, demonstrated, disulfide and esterase cleavable moieties function to liberate the drug inside of the targeted cell. Moreover, it shows that many different moieties W are capable of forming a ring together with the spacer E, Y1 and P after liberation (i.e. cleavage of Z). In particular, these results demonstrate that, for example, a liberated thiol or a liberated alcohol is capable of forming a 3-8-membered ring thereby facilitating traceless release of the drug attached via X to the phosphorus atom, when delivered into the targeted cell via a receptor binding molecule RBM.

### 9F. In Vitro Efficacy of ADCs Comprising Different Linkers L

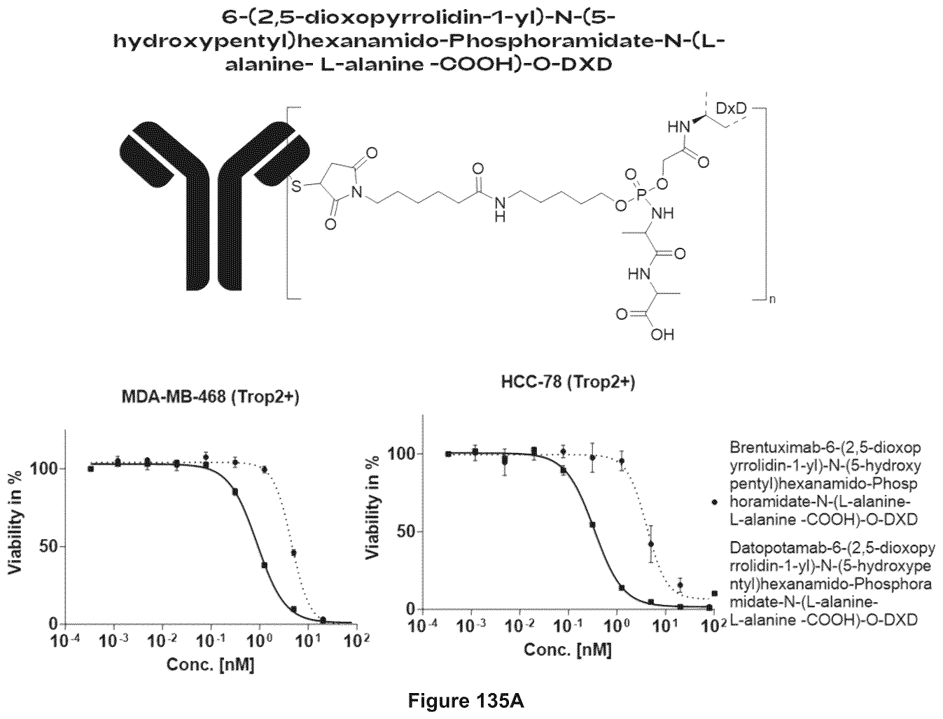

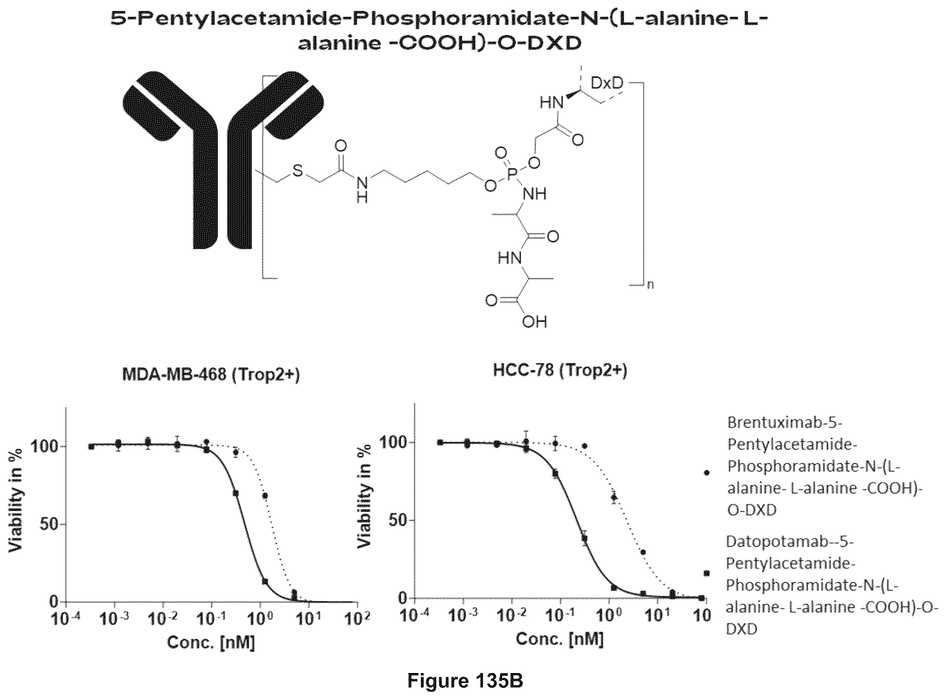

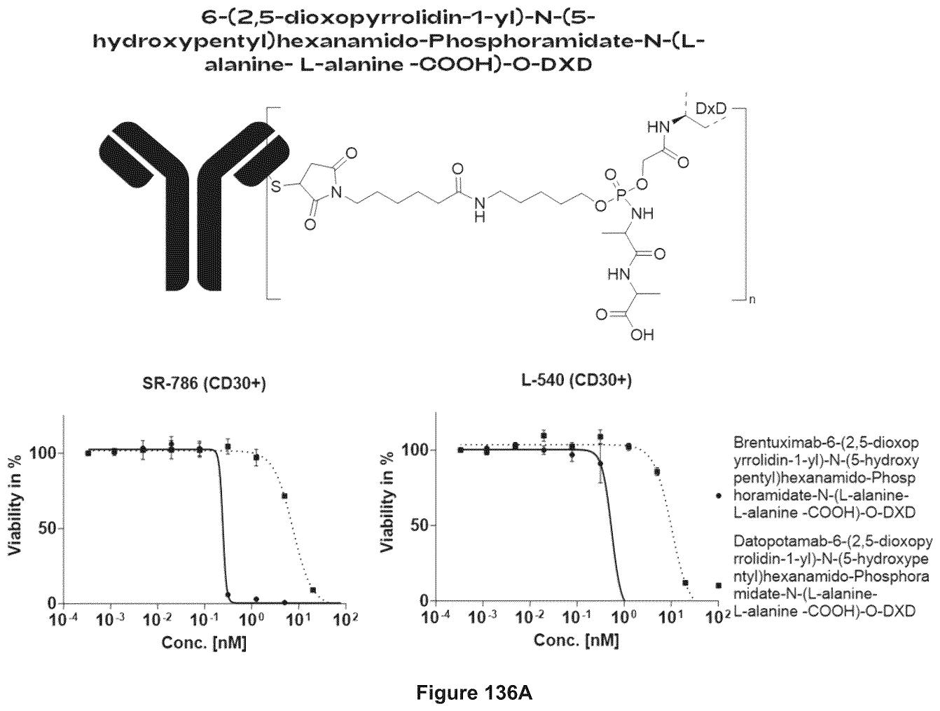

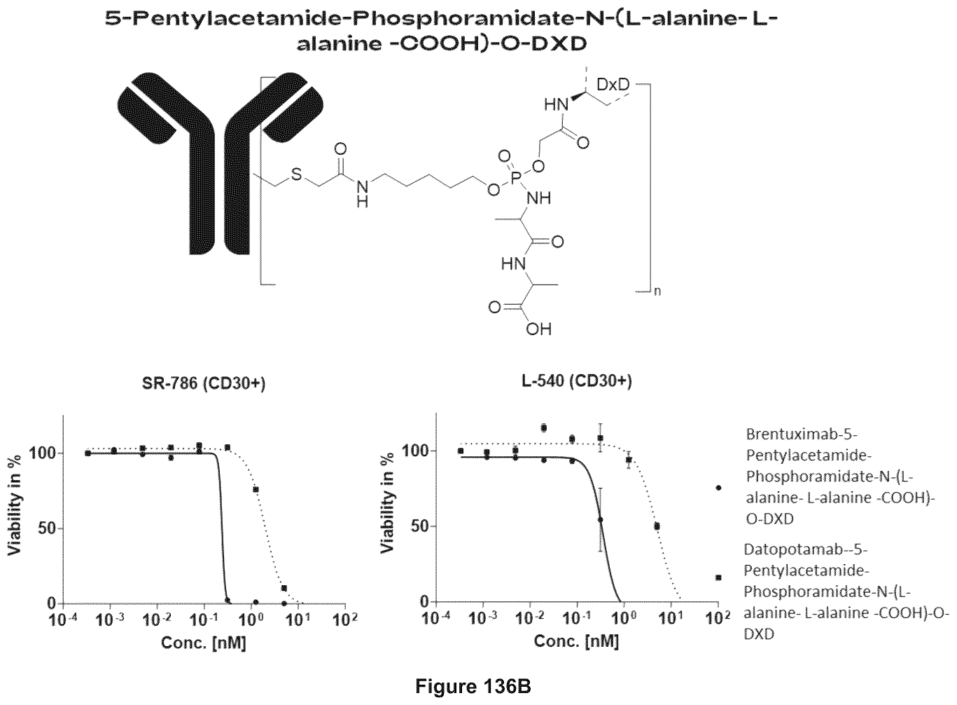

In order to demonstrate variability of the linker L, the inventors synthesized DXD conjugates using maleimide and iodoacetamide based conjugation methods to thereby provide conjugates comprising linkers L that have an entirely different chemical nature. The structures of the tested ADC variants are depicted in FIGS. 135A and 135B. Effectiveness of selective targeted release of the drug was investigated using these ADC variants in accordance with embodiments of the invention comprising DXD as drug and the described linker systems from Datopotamab (Trop2-targeted) and Brentuximab (non-targeted isotype control) and the results against the two different Trop2-positive cell lines (MDA-MB-468) and (HCC-78) are shown in FIGS. 135A and 135B. Effectiveness of selective targeted release of the drug was further investigated using said ADCs in accordance with the described embodiments of the invention comprising DXD as drug and the described linker systems from Brentuximab (CD30-targeted) and Datopotamab (non-targeted isotype control) against the two different CD30-positive cell lines (L-540) and (SR-786) and the results are shown in the FIGS. 136A and 136B. These results clearly demonstrate selective target mediated delivery by the conjugated antibody and effective release of the drug. More importantly, these results underline the variability of the chemical nature of the linker L, in particular that stable attachment and release of a given drug is fully independent of and compatible with different conjugation methods used for conjugation to the receptor binding molecule RBM.

### Example 10: In Vitro Efficacy of ADC Variants Comprising Different Drug Moieties and Variations in Other Chemical Groups

### 10A. In Vitro Efficacy of ADCs Comprising Different Dihydroorotate Dehydrogenase (DHODH) Inhibitors as Drug

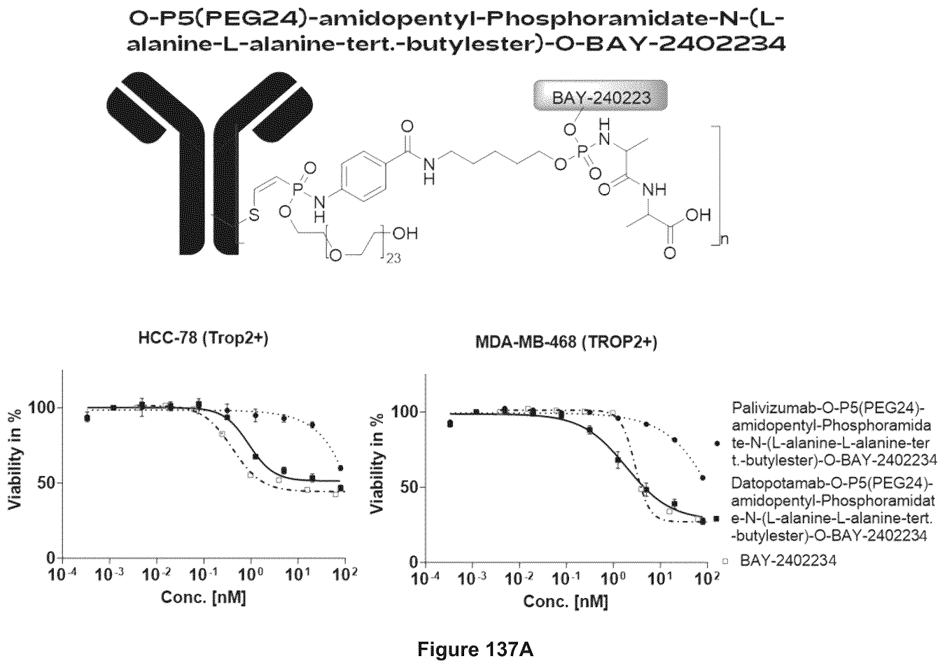

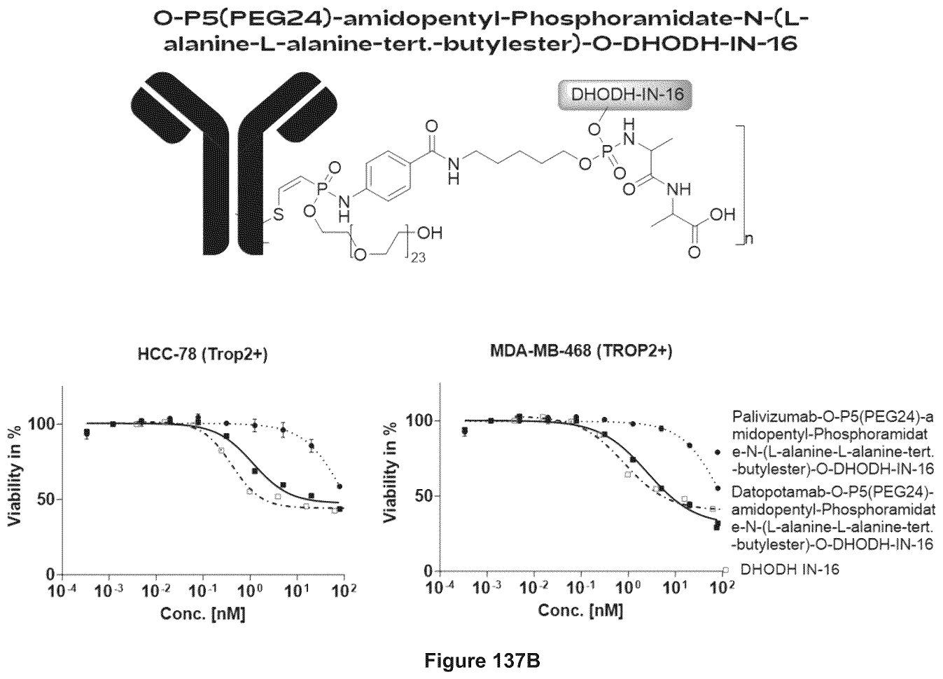

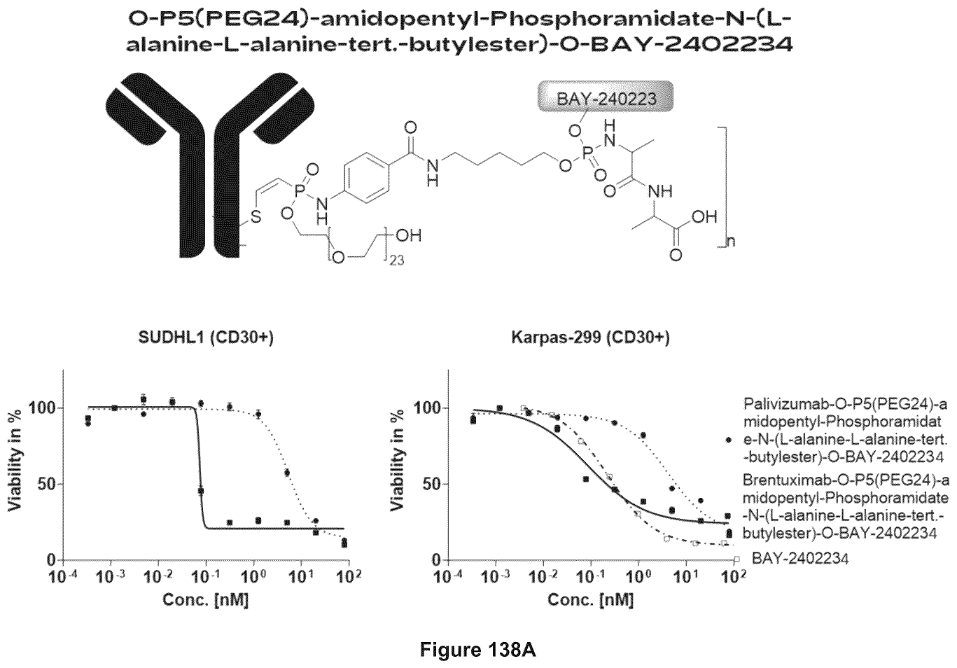

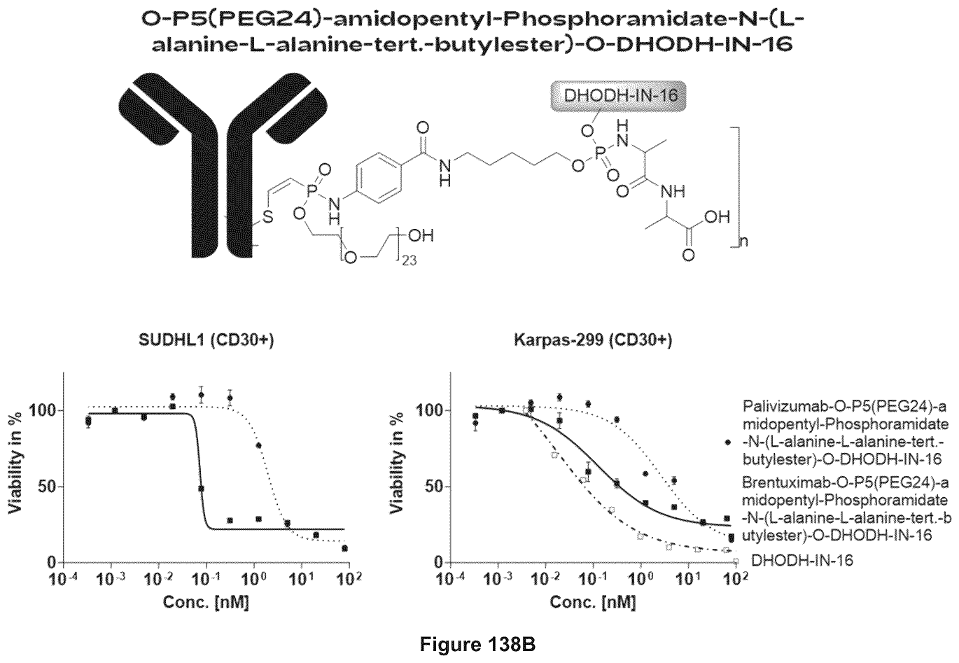

In order to further demonstrate applicability of the system to a wide variety of different drugs, the inventors synthesized conjugates comprising different dihydroorotate dehydrogenase (DHODH) inhibitors as drug. According to the knowledge of the inventors' knowledge, successful conjugation of DHODH inhibitors as payloads is unprecedented in literature. In particular, the inventors synthesized a conjugate with the DHODH inhibitor BAY-2402234 and a conjugate with the DHODH inhibitor DHODH-IN16. The structures are depicted in FIGS. 137A and 137B and FIGS. 138A and 138B. Effectiveness of selective targeted release of the drugs was investigated with these ADCs in accordance with embodiments of the invention comprising BAY-2402234 or DHODH-IN16 as drug and the described linker systems from Datopotamab (Trop2-targeted) and Palivizumab (non-targeted isotype control) against the two different Trop2-positive cell lines (MDA-MB-468) and (HCC-78); the results are shown in FIGS. 137A and 137B. Effectiveness of selective targeted release of the drugs was further investigated using said ADC embodiments of the invention comprising BAY-2402234 or DHOSH-IN16 as drug and the described linker systems from Brentuximab (CD30-targeted) and Palivizumab (non-targeted isotype control) against the two different CD30-positive cell lines (SUDHL1) and (Karpas-299); the results are shown in FIGS. 138A and 138B. These results once more highlight superiority and versatile applicability of the system described herein to different drugs. Notably, nowhere before has an DHODH inhibitor been described as ADC payload in literature, let alone its successful targeted delivery. In other words, these results clearly demonstrate that the system of the invention can be used to make all kinds of drugs, including novel drugs with new modes of action, available for selective targeted drug delivery.

### 10B. In Vitro Efficacy of ADCs Comprising the Drug Paclitaxel

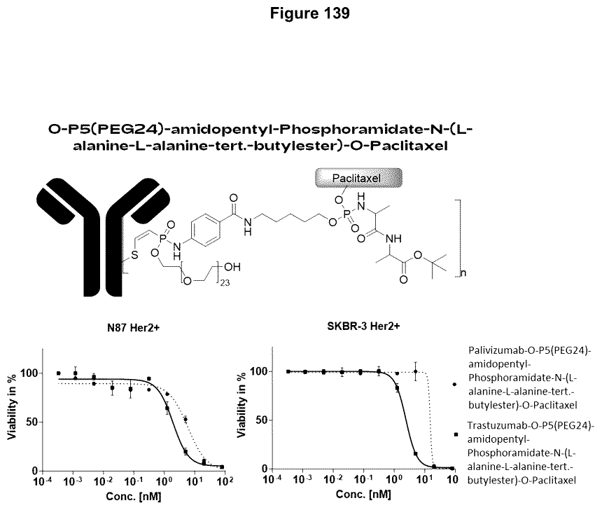

In order to further demonstrate versatility of the system to different kinds of drugs, the inventors synthesized a Paclitaxel conjugate. The structure is depicted in FIG. 139. Effectiveness of selective targeted release of the drug was investigated with this ADC in accordance with embodiments of the invention comprising Paclitaxel as drug and the shown linker system from Trastuzumab (Her2-targeted) and Palivizumab (non-targeted isotype control) against the two different HER2-positive cell lines (N₈₇) and (SKBR3); the results are shown in FIG. 139 These results again underline the versatile applicability of the system described herein to make different drugs available for selective targeted drug delivery mediated by the receptor binding molecule RBM.

### 10C. In Vitro Efficacy of ADCs Comprising the Drug Ganetespib and Variations in Cleavable Group Z

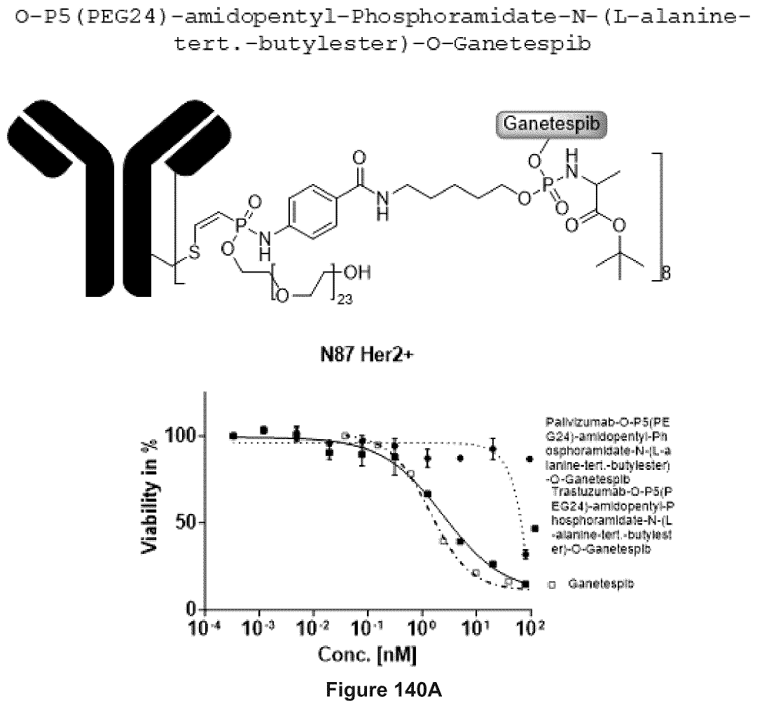

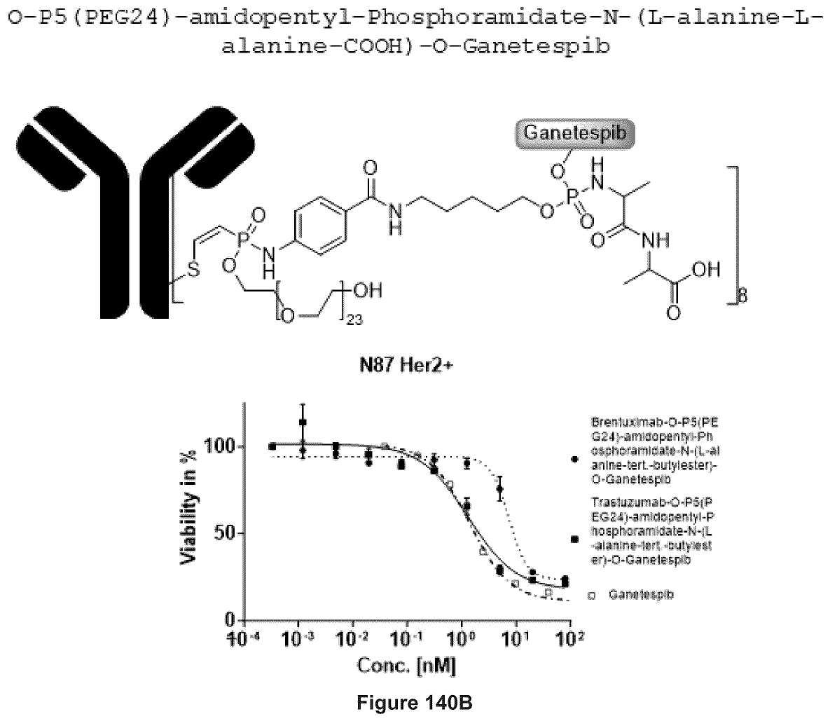

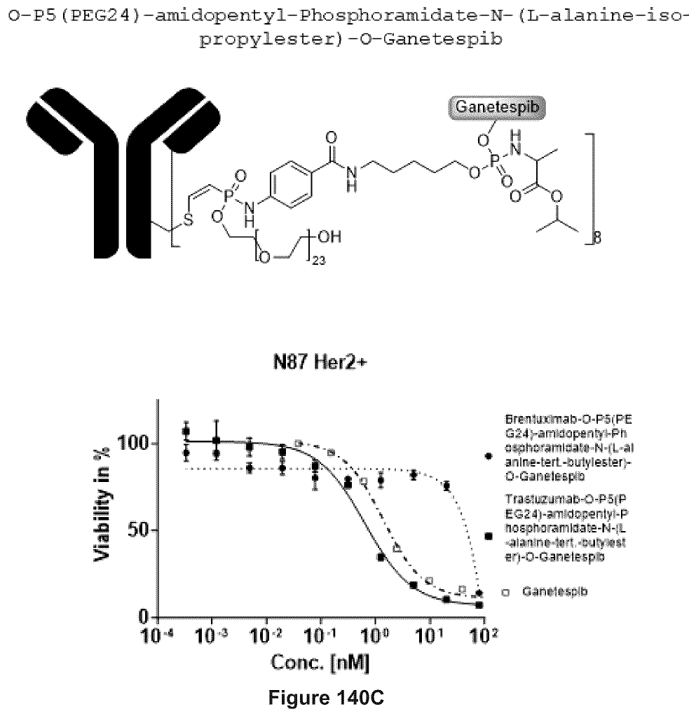

In order to investigate the effectiveness of release of varying drug molecules and variability of chemical entities within the system, the inventors synthesized conjugates comprising the HSP90 inhibitor Ganetespib as drug and different cleavable groups Z. Tested ADC variants comprised as cleavable group Z: (i) NH—CHMe-COOH; (ii) isopropylester (iPr); and (iii) tert-butyl ester (tBu). The structures of the ADCs are depicted in FIGS. 140A to 140C. Effectiveness of selective targeted release of the drug was investigated with these ADCs in accordance with embodiments of the invention comprising Ganetespib as drug and the shown linker systems from Trastuzumab (Her2-targeted) and Brentuximab or Palivizumab (non-targeted isotype control) against the HER2-positive cell line (N₈₇); the results are shown in FIGS. 140A to 140C. These results once more prove the variability of the chemical nature of the individual chemical entities in accordance with the invention (cleavable group Z in this case) and the versatile applicability of the system described herein for making different drugs (Ganetespib in this case) available for selective targeted drug delivery mediated by the receptor binding molecule RBM.

### 10D. In Vitro Efficacy of ADCs Comprising an Eukaryotic Translation Initiation Factor

4E (eIF4E) Inhibitor as Drug and Variations in Cleavable Group Z

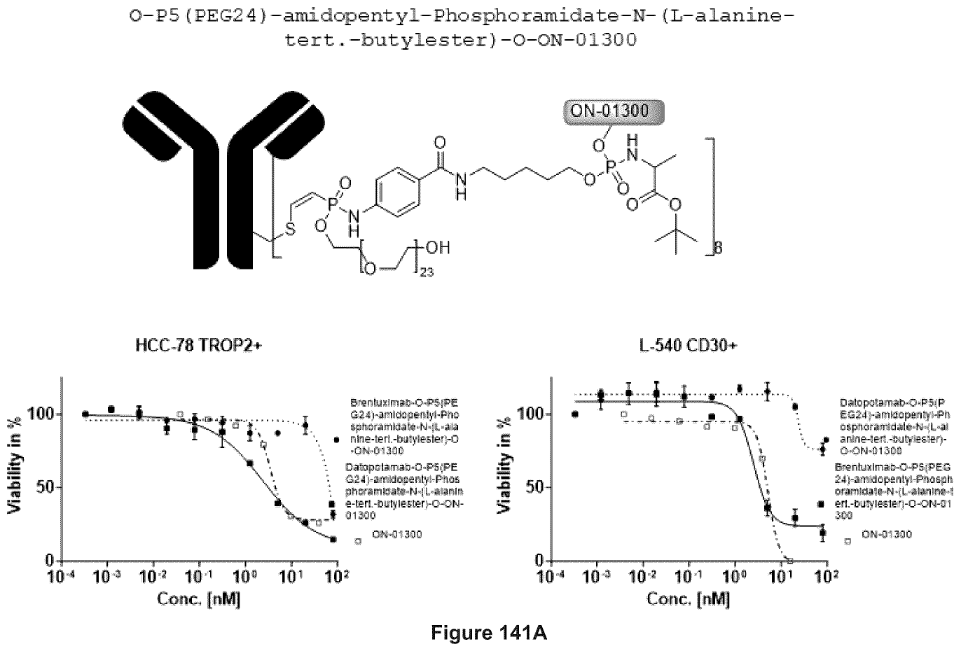

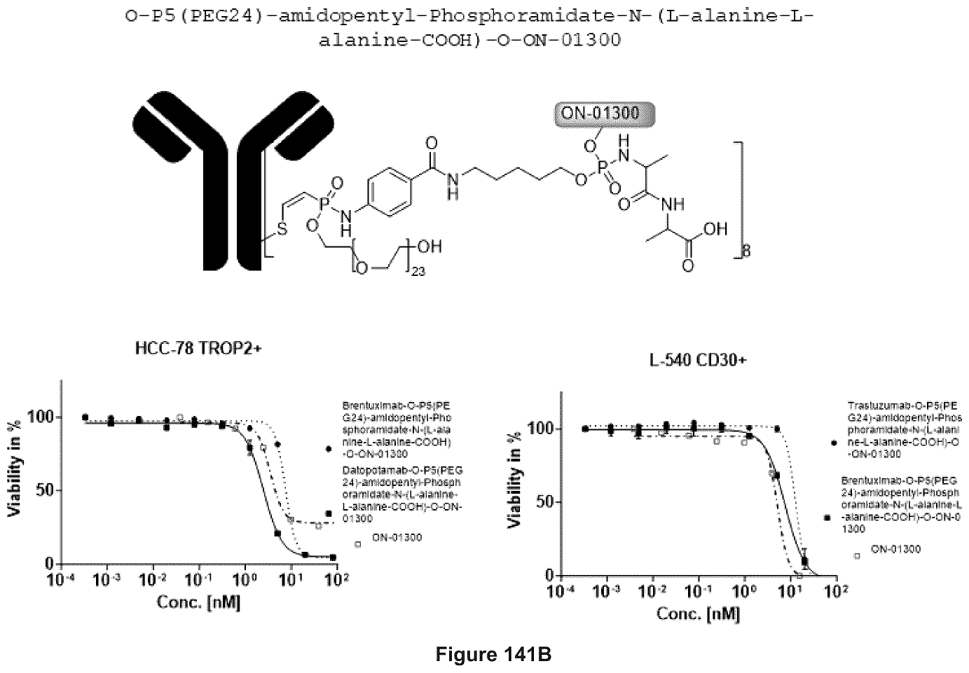

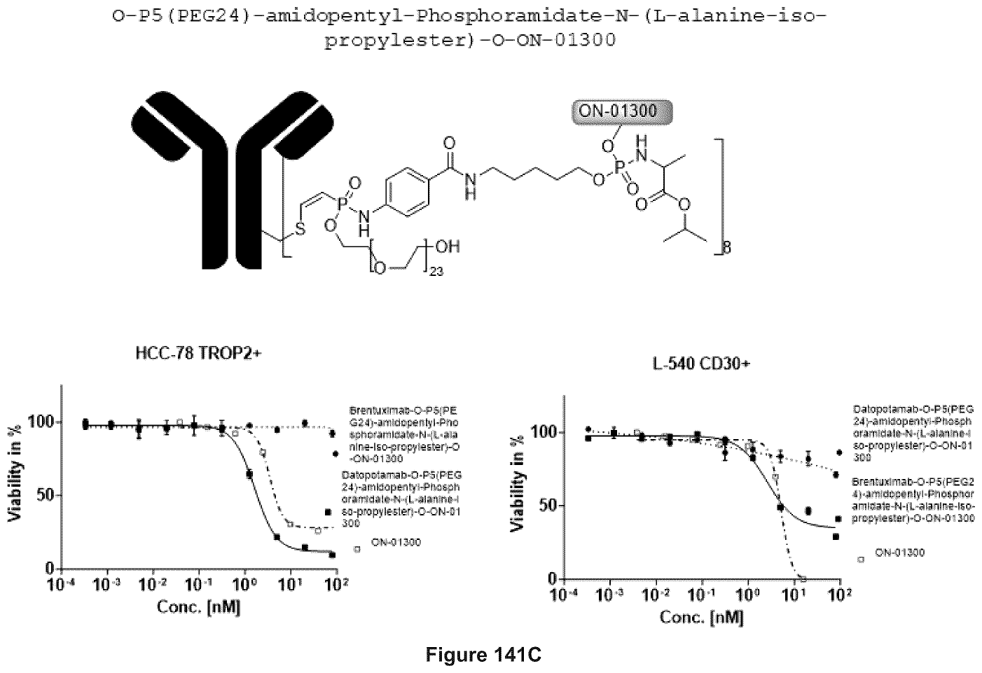

In order to further investigate the effectiveness of release of varying drug molecules and variability of chemical entities within the system, the inventors synthesized conjugates comprising another unprecedented payload, namely the eIF4E inhibitor ON-1300 as drug, and different cleavable groups Z. Tested ADC variants comprised as cleavable group Z: (i) tert-butyl ester (tBu); (ii) NH—CHMe-COOH; and (iii) isopropylester (iPr). The structures of the ADCs are depicted in FIGS. 141A to 141C. Effectiveness of selective targeted release of the drug was investigated with these ADCs in accordance with embodiments of the invention comprising the eIF4E inhibitor ON-1300 as drug and the shown linker systems from Datopotamab (Trop2-targeted) and Brentuximab (non-targeted isotype control in TROP2-positive cell lines) against Trop2-positive cell line (HCC-78); the results are shown in FIGS. 141A to 141C. The results of ADCs in accordance with embodiments of the invention comprising ON-1300 as drug and the described linker systems from Brentuximab (CD30-targeted) and Datopotamab (non-targeted isotype control in CD30-positive cell lines) against the CD30-positive cell line (L-540) are also shown in FIGS. 141A to 141C. These results once more highlight superiority, variability of the chemical nature of the individual chemical entities in accordance with the invention and versatile applicability of the system described herein to different drugs. Noteworthy, according to the inventors' knowledge, there is again no literature precedence of any ADC having an eIF4E inhibitor as payload, let alone demonstration of successful targeted delivery of such an elFf4E inhibitor. Thus, these results once again clearly demonstrate that different chemical entities in accordance with the invention can be used as constituents of the system and that the system provides the unique opportunity to make all kinds of drugs, including novel drugs with new modes of action, available for selective targeted drug delivery.

### 10E. Investigation of the Release Mechanism of the Linker Systems Described in the Invention

In order to further underline the principle mechanism of the release of X-D, as depicted in the description of the current invention, an esterase cleavable construct was incubated with an esterase and the resulting fragments were analyzed via LC/MS. The experiment has been conducted as follows:

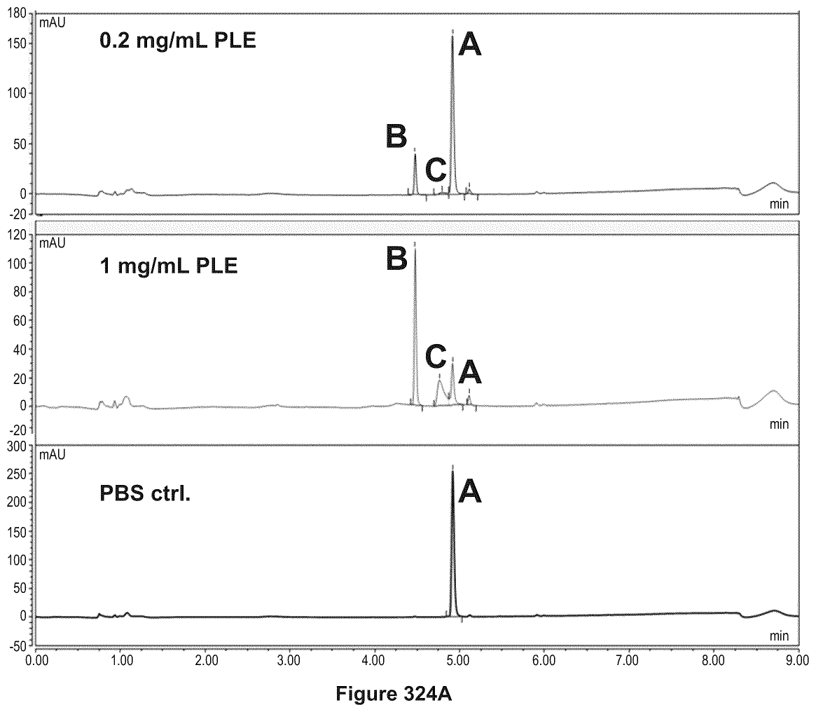

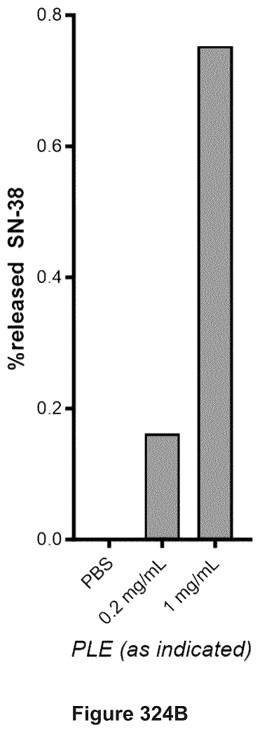

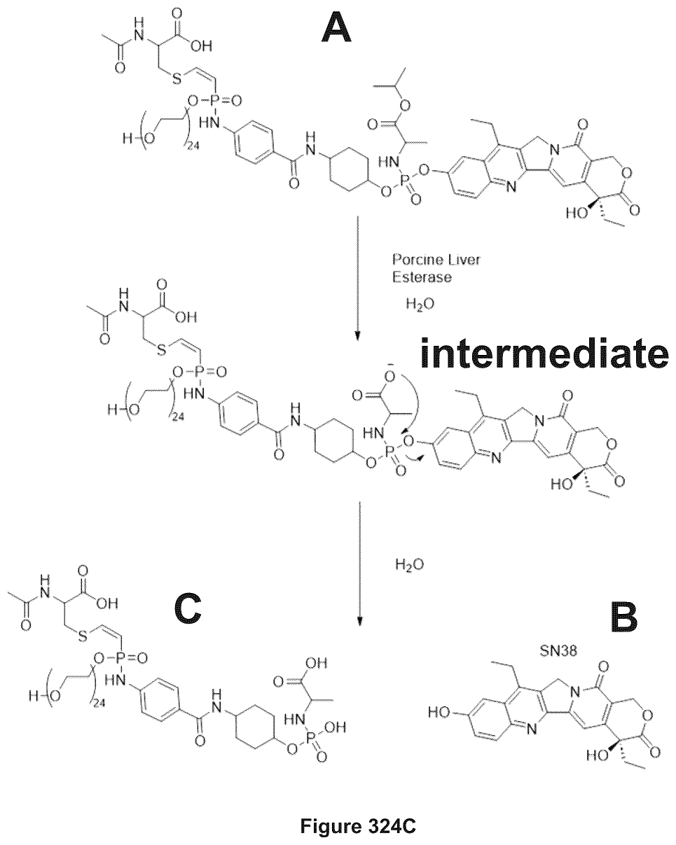

To a solution of N-Acetyl-Cysteine (10 equiv., 50 mM in TE, buffer) was added NaOH (10 equiv., from 1 mM) to adjust the pH to 8. A solution of O—P5(PEG24)-amidocyclohexyl-Phosphoramidate-N-(L-alanine-iso-propylester)-O—SN38 (40 mM in DMSO) was added dropwise to the solution at r.t. The mixture was kept at r.t. for 1 h. Purification by prep HPLC (MeCN/H2O/0.1% TFA) yielded the title N-Acetyl-Cysteine-capped O—P5(PEG24)-amidocyclohexyl-Phosphoramidate-N-(L-alanine-iso-propylester)-O—SN38 (Compound A in FIG. 324C) A. A solution of A (1 mM in DMSO) was added into Dulbecco's PBS to achieve a final concentration of 0.02 mM in PBS (5% DMSO). To this mixture was added a solution of PLE (10 mg/mL; 0.15 u/uL) to achieve the indicated final concentration of 0.2 mg/mL and 1 mg/mL respectively. A control reaction did not contain PLE. The mixture was incubated at 37° C. for 24 h and analysed by analytical LC-MS (MeCN/H2O/0.1% FA). The data is interpreted in a way that the incubation with PLE leads to cleavage of the iso-propylester, which is PLE concentration dependent. The cleavage leads to an intramolecular ring formation at the phosphorous kicking out SN38 B. The remaining cyclic intermediate is hydrolysed spontaneously to give fragment C. A, B and C were identified by MS and the structures are depicted in FIG. 324C.

This experiment provides further evidence that the mechanism described herein holds true, since it confirms that unmodified SN38 is tracelessly released in the presence of an Esterase and not if an Esterase is not present. The formation of Side product B further supports the hypothesis of cyclisation of the moeity W that is released after cleavage of Z with the Phosphorus atom leads to a traceless release of X-D.

1. A conjugate having the formula (I): 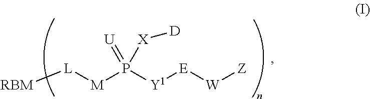
or a pharmaceutically acceptable salt or solvate thereof;
wherein:
RBM is a receptor binding molecule;
L is a linker;
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group;
W is a moiety which, after cleavage of the group Z, is capable of forming a ring together with the spacer E, Y¹ and the phosphorus; and
n is an integer ranging from 1 to 20.

2. The conjugate according to claim 1 having the formula (Ia): 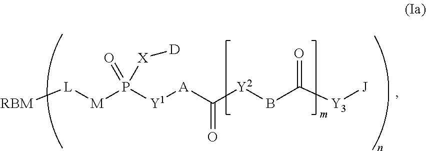
or a pharmaceutically acceptable salt or solvate thereof;
wherein:
RBM, L, M, X, D, Y¹ and n are as defined in claim 1;
A is CR^{A30}R^{A31}; or
A is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl;
R^{A30} and R^{A31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{A36} and CONR^{A36}R^{A37} wherein R^{A36} and R^{A37}, which may be the same or different, are independently selected from (C₁-C₈)alkyl, (C₁-C₈)alkylene(C₆-C₁₀)aryl or (C₆-C₁₀)aryl; optionally R^{A30} and R^{A31} can together form a 3 to 8-membered ring;
Y² is NR^{B20}, O, S, or CR^{B21}R^{B22};
R^{B20} is selected from the group consisting of hydrogen, (C₁-C₈)alkyl, (C₆-C₁₀)aryl, and C₁-C₈)alkylene(C₆-C₁₀)aryl;
R^{B21} and R^{B22} are each independently selected from the group consisting of hydrogen, (C₁-C₈)alkyl, (C₆-C₁₀)aryl, and (C₁-C₈)alkylene(C₆-C₁₀)aryl;
B is, each independently, CR^{B30}R^{B31}; or
B is, each independently, (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37}, wherein R^{B36} and R^{B37}, which may be the same or different, are independently selected from (C₁-C₈)alkyl, (C₁-C₈)alkylene(C₆-C₁₀)aryl or (C₆-C₁₀)aryl;
R^{B30} and R^{B31} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{B36} and CONR^{B36}R^{B37} wherein R^{B36} and R^{B37}, which may be the same or different, are independently selected from (C₁-C₈)alkyl, (C₁-C₈)alkylene(C₆-C₁₀)aryl or (C₆-C₁₀)aryl; optionally R^{B30} and R^{B31} can together form a 3 to 8-membered ring;
m is an integer ranging from 0 to 15;
Y³ is O, NR^{C40}, S, or absent;
R^{C40} is selected from the group consisting of hydrogen, (C₁-C₈)alkyl, (C₆-C₁₀)aryl, and (C₁-C₈)alkylene(C₆-C₁₀)aryl;
J is 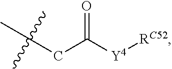
wherein [symbol] indicates the attachment to Y³;
C is CR^{C50}R^{C51}; or
C is (C₁-C₈)alkylene, wherein the (C₁-C₈)alkylene may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl;
R^{C50} and R^{C51} are each independently selected from the group consisting of hydrogen, (C₁-C₈)alkyl, (C₃-C₈)cycloalkyl, (C₂-C₈)alkenyl, (C₅-C₈)cycloalkenyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C36} and CONR^{C36}R^{C37}, wherein R^{C36} and R^{C37}, which may be the same or different, are independently selected from (C₁-C₈)alkyl, (C₁-C₈)alkylene(C₆-C₁₀)aryl or (C₆-C₁₀)aryl; optionally R^{C50} and R^{C51} can together form a 3 to 8-membered ring;
Y⁴ is O, NR^{C53}, S, CR^{C54}R^{C55}, or absent;
R^{C52} is selected from the group consisting of hydrogen, (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C56} and CONR^{C56}R^{C57} wherein R^{C56} and R^{C57}, which may be the same or different, are independently selected from (C₁-C₈)alkyl, (C₁-C₈)alkylene(C₆-C₁₀)aryl or (C₆-C₁₀)aryl;
R^{C53} is selected from the group consisting of hydrogen, (C₁-C₈)alkyl, (C₆-C₁₀)aryl, and (C₁-C₈)alkylene(C₆-C₁₀)aryl;
R^{C54} and R^{C55} are each independently selected from the group consisting of hydrogen, (C₁-C₈)alkyl, (C₆-C₁₀)aryl, and (C₁-C₈)alkylene(C₆-C₁₀)aryl; or
J is selected from the group consisting of (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl, and (C₁-C₈)alkylene(C₆-C₁₀)aryl; wherein each (C₁-C₈)alkyl, (C₃-C₈)cycloalkyl, (C₂-C₈)alkenyl, (C₅-C₈)cycloalkenyl, (C₃-C₈)heterocyclyl, (C₆-C₁₀)aryl or (C₁-C₈)alkylene(C₆-C₁₀)aryl may be optionally substituted with one or more substituents selected from the group consisting of (C₁-C₈)alkyl, halo, hydroxy, (C₁-C₈)alkoxy, amino, (C₁-C₈)alkylamino, di(C₁-C₈)alkylamino, SH, (C₁-C₈)alkylthio, (C₃-C₈)heterocyclyl, carboxylate and esters thereof, carboxy(C₁-C₈)alkyl, CONHR^{C46} and CONR^{C46}R^{C47} wherein R^{C46} and R^{C47}, which may be the same or different, are independently selected from (C₁-C₈)alkyl, (C₁-C₈)alkylene(C₆-C₁₀)aryl or (C₆-C₁₀)aryl.

3. The conjugate according to claim 2, wherein the conjugate has formula (Ia1): 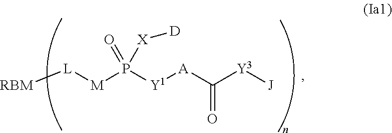
or a pharmaceutically acceptable salt or solvate thereof;
wherein RBM, L, M, X, D, Y¹, A, Y³, J and n are as defined in claim 2.

4. The conjugate according to claim 3, wherein A is CR^{A30}R^{A31} wherein R^{A30} is hydrogen and R^{A31} is hydrogen or (C₁-C₈)alkyl, such as CH₃.

5. The conjugate according to claim 4, wherein Y³ is O and J is (C₁-C₈)alkyl.

6. The conjugate according to claim 4, wherein Y³ is NH; and
J is 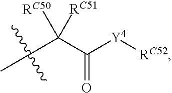
wherein R^{C50} is hydrogen; R^{C51} is hydrogen or (C₁-C₈)alkyl, such as CH₃; Y⁴ is O; and R^{C52} is hydrogen or (C₁-C₈)alkyl.

7. The conjugate of claim 1 having the formula (Ib): 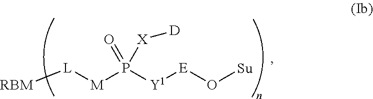
or a pharmaceutically acceptable salt or solvate thereof;
wherein:
RBM, L, M, X, D, Y¹ and n are as defined in claim 1;
E is a spacer;
Su is a sugar moiety which is bound to the oxygen atom via a cleavable bond; and
wherein the oxygen, after cleavage of the sugar moiety Su, is capable of forming a ring together with the spacer E, Y¹ and the phosphorus.

8. The conjugate of claim 1 having the formula (Ic): 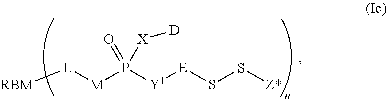
or a pharmaceutically acceptable salt or solvate thereof;
wherein:
RBM, L, M, X, D, Y¹ and n are as defined in claim 1;
E is a spacer;
Z* is an optionally substituted aliphatic residue or an optionally substituted aromatic residue; and
wherein the sulfur bound to the spacer E, after cleavage of the disulfide bond, is capable of forming a ring together with the spacer E, Y¹ and the phosphorus.

9. The conjugate of claim 1 having the formula (Ie): 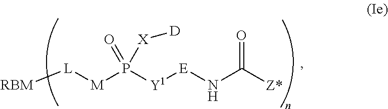
or a pharmaceutically acceptable salt or solvate thereof;
wherein:
RBM, L, M, X, D, Y¹ and n are as defined in claim 1;
E is a spacer;
Z* is an optionally substituted aliphatic residue or an optionally substituted aromatic residue; and
wherein the nitrogen atom bound to the spacer E, after cleavage of the acetyl bond, is capable of forming a ring, preferably a four- to seven-membered ring, more preferably a five-or six-membered ring, together with the spacer E, Y¹ and the phosphorus.

10. The conjugate of claim 1 having the formula (If): 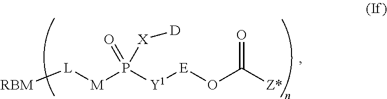
or a pharmaceutically acceptable salt or solvate thereof;
wherein:
RBM, L, M, X, D, Y¹ and n are as defined in claim 1;
E is a spacer;
Z* is an optionally substituted aliphatic residue or an optionally substituted aromatic residue; and
wherein the oxygen atom bound to the spacer E, after cleavage of the acetyl bond, is capable of forming a ring, preferably a four- to seven-membered ring, more preferably a five-or six-membered ring, together with the spacer E, Y¹ and the phosphorus.

11. The conjugate according to claim 1, wherein the receptor binding molecule (RBM) is selected from the group consisting of an antibody, an antibody fragment, a proteinaceous binding molecule with antibody-like binding properties, an aptamer, and a small molecule; preferably the receptor binding molecule is an antibody.

12. The conjugate according to claim 1, wherein X is O or NH.

13. The conjugate according to claim 1, wherein the drug moiety D is selected from the group consisting of a Mitotic Spindle-Inhibitor such as (−)-Epipodophyllotoxin, a Dehydrogenase A-Inhibitor such as (R)-GNE-140, a Kinase-Inhibitor such as (S)-3-Hydroxy Midostaurin and (R)-3-Hydroxy Midostaurin, a BET-Inhibitor such as ABBV-744, a Estrogene Receptor Agonist such as Acolbifene, a Wee1-Inhibitor such as Adavosertib, a HSP90-Inhibitor such as Alvespimycin, a Kinase-Inhibitor such as ARS-1620, a FGFR-Inhibitor such as ASP5878, a MCT1-Inhibitor such as AZD3965, a mTOR-Inhibitor such as AZD-8055, a Kinase-Inhibitor such as Belizatinib, a HIF-2a inhibitor such as Belzutifan, a BCL-Inhibitor such as BM-1197, a VEGFR-Inhibitor such as Brivanib, a STAT3-Inhibitor such as C188, a anti tumor such as CB1151, a Kinase-Inhibitor such as Dasatinib, a EGFR-Inhibitor such as DBPR112, a CDK-Inhibitor such as Dinaciclib, a TRPC4 and TRCP5 Channel Activator such as Englerin A, a PRMT-Inhibitor such as EPZ015666, a Topoisomerase-Inhibitor such as Etoposide, a mTOR-Inhibitor such as Everolimus, a Methyltransferase-Inhibitor such as EZM 2302, a CDK-Inhibitor such as Fadraciclib, a USP7-Inhibitor such as FT671, a Estrogene Receptor Agonist such as Fulvestrant, a Estrogene Receptor Agonist such as Fulvestrant, a HSP90-Inhibitor such as Geldanamycin, a Estrogene Receptor Agonist such as GNE-274, a Kinase-Inhibitor such as GNE-493, a PRMT-Inhibitor such as GSK3326595, a Kinase-Inhibitor such as Hypothemycin, a CDK-Inhibitor such as IIIM-290, a DNA alkylator such as Illudin S, a Kinase-Inhibitor such as Ilorasertib, a Kinase-Inhibitor such as Larotrectinib, a Kinase-Inhibitor such as Larotrectinib, a IGF-1-Inhibitor such as Linsitinib, a PRMT-Inhibitor such as LLY-283, a HSP90-Inhibitor such as Luminespib, a FGFR-Inhibitor such as LY2874455, a Kinase-Inhibitor such as Mirdametinib, a Kinase-Inhibitor such as MRTX1133, a Kinase-Inhibitor such as MRTX1133, a Kinase-Inhibitor such as Ningetinib, DNA minor groove binder such as Lurbinectidin or Trabectidin, a HSP90-Inhibitor such as NMS-E973, a Ribonucleotide Reductase-Inhibitor such as NSAH, a PLK1-Inhibitor such as Onvansertib, a mTOR-Inhibitor such as Palomid 529, a Kinase-Inhibitor such as PD166326, a NEDD8-Inhibitor such as Pevonedistat, a Kinase-Inhibitor such as PF-04217903, a Kinase-Inhibitor such as PF-06843195, a HSP90-Inhibitor such as PI-103, a Methyltransferase-Inhibitor such as Pinometostat, a Topoisomerase-Inhibitor such as PNU-159682, a Topoisomerase-Inhibitor such as Podofilox, a HDAC-Inhibitor such as QTX125, a mTOR-Inhibitor such as Rapamycin, a Tankyrase-Inhibitor such as RK-287107, a Kinase-Inhibitor such as RO4987655, a Kinase-Inhibitor such as RP-3500, a BCL-Inhibitor such as S55746, a BCL-Inhibitor such as S65487, a EGFR-Inhibitor such as SDZ281-977, a Kinase-Inhibitor such as SU14813, a Kinase-Inhibitor such as TC-A 2317, a Kinase-Inhibitor such as Teleocidin A1, a Ribonucleotide Reductase-Inhibitor such as Tezacitabine, a Kinase-Inhibitor such as TG 100572, a Inhibitor of RNA splicing such as Thailanstatin A, a Kinase-Inhibitor such as UNC5293, a Kinase-Inhibitor such as UNC5293, a HSP90-Inhibitor such as VER-50589, a eIF4A-Inhibitor such as Zotatifin and analogues or prodrugs thereof; preferably wherein the drug moiety is selected from the following (a) to (n), wherein
(a) the camptothecin compound is selected from the group consisting of DXD, SN38, exatecan, camptothecin, topotecan, irinotecan, belotecan, lurtotecan, rubitecan, silatecan, cositecan, and gimatecan, more preferably the camptothecin compound is DXD or SN38;
(b) the TOPK inhibitor is OTS-964;
(c) the CDK inhibitor is ganetespib or Roniciclib;
(d) the bromodomain inhibitor is birabresib;
(e) the HSP90 inhibitor is SNX-2112;
(f) the ribonucleotide reductase inhibitor is gemcitabine;
(g) the Aurora B kinase inhibitor is barasertib;
(h) the HSp 70 inhibitor is Triptolide;
(i) the nicotinamide phosphoribosyltransferase (Nampt) inhibitor is Nampt-IN-1;
(j) the eukaryotic Translation Initiation Factor 4E (eIF4E) inhibitor is ON-01300;
(k) the dihydroorotate dehydrogenase (DHODH) inhibitor is Bay-240223 or DHODH-IN-16;
(I) the taxane is selected from the group consisting of Paclitaxel, albumin-bound Paclitaxel (nab-Paclitaxel), Docetaxel, Cabazitaxel and Abraxan, more preferably the taxan is Paclitaxel;
(m) the auristatin is monomethyl auristatin E (MMAE) or monomethyl auristatin F (MMAF);
(n) the KSP inhibitor is selected from the group consisting of Ispinesib (SB-715992), SB743921, AZ 3146, GSK923295, BAY 1217389, MPI-0479605 and ARQ 621.

14. The conjugate according to claim 1, wherein
(i) M is O, X is O and Y¹ is NH; or
(ii) M is O, X is NH and Y¹ is O; or
(iii) M is NH, X is O and Y¹ is O.

15. The conjugate according to claim 1, wherein the linker L has the formula (L-I): 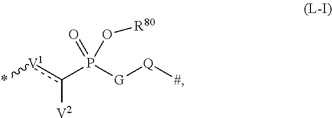
wherein: [symbol]
is a double bond; or [symbol]
is a bond;
V² is absent when [symbol] is a double bond; or
V² is selected from the group consisting of hydrogen, (C₁-C₈)alkyl, (C₆-C₁₀)aryl, and
(C₁-C₈)alkylene(C₆-C₁₀)aryl when [symbol] is a bond;
V¹ is R^{V11—C} when [symbol] is a double bond; or
V¹ is 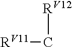
when [symbol] is a bond;
G is NR^{G70}, S, O, or CR^{G71}R^{G72};
Q is a connector unit;
R^{V11} is selected from the group consisting of hydrogen, (C₁-C₈)alkyl, (C₆-C₁₀)aryl, and (C₁-C₈)alkylene(C₆-C₁₀)aryl;
R^{V12} is selected from the group consisting of hydrogen, (C₁-C₈)alkyl, (C₆-C₁₀)aryl, and (C₁-C₈)alkylene(C₆-C₁₀)aryl;
R^{G70} is selected from the group consisting of hydrogen, (C₁-C₈)alkyl, (C₆-C₁₀)aryl, and (C₁-C₈)alkylene(C₆-C₁₀)aryl;
R^{G71} and R^{G72} are each independently selected from the group consisting of hydrogen, (C₁-C₈)alkyl, (C₆-C₁₀)aryl, and (C₁-C₈)alkylene(C₆-C₁₀)aryl;
R⁸⁰ is an optionally substituted aliphatic residue or an optionally substituted aromatic residue;
* indicates the attachment to the receptor binding molecule (RBM); and
# indicates the attachment to M.

16. The conjugate according to claim 15, wherein G is NH, and
Q is selected from (a) or (b):
(a) 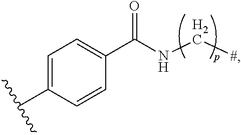
wherein:
p is an integer ranging from 2 to 20; [symbol]
indicates the attachment to G; and
# indicates the attachment to M; or
(b) 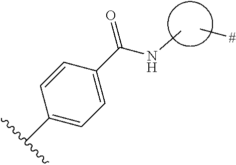
wherein [symbol] is a (C₃-C₈)carbocycle, (C₆-C₁₀)aryl (phenyl), a five- or six-membered heterocyclic ring comprising 1, 2 or 3 heteroatoms independently selected from the group consisting of N, O and S; preferably (C₃-C₈)cycloalkyl; more preferably 5-, 6-, or 7-membered cycloalkyl, even more preferably cyclohexyl; [symbol]
indicates the attachment to G; and
# indicates the attachment to M.

17. A compound having the formula (II): 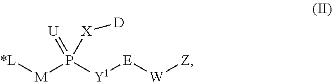
or a pharmaceutically acceptable salt or solvate thereof;
wherein:
L* is a linker capable of forming a covalent attachment to a receptor binding molecule (RBM);
M is O, NR^{M60} or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group; and
W is a moiety which, after cleavage of the group Z, is capable of forming a ring together with the spacer E, Y¹ and the phosphorus atom.

18. A method of preparing a conjugate of formula (I), said method comprising:
reacting a compound of formula (II) 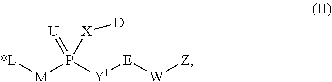
or a pharmaceutically acceptable salt or solvate thereof;
wherein:
L* is a linker capable of forming a covalent attachment to a receptor binding molecule (RBM);
M is O, NR^{M60}, or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group; and
W is a moiety which, after cleavage of the group Z, is capable of forming a ring together with the spacer E, Y¹ and the phosphorus;
with a receptor binding molecule (RBM) having a functional group reactive towards L* of a compound of formula (II), thereby forming a covalent bond between the receptor binding molecule (RBM) and the linker (L) to result in a conjugate of formula (I): 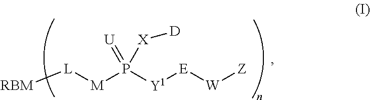
or a pharmaceutically acceptable salt or solvate thereof;
wherein:
RBM is a receptor binding molecule;
L is a linker;
M is O, NR^{M60}, or S;
R^{M60} is selected from the group consisting of hydrogen, (C₁-C₈)alkyl, (C₆-C₁₀)aryl, and (C₁-C₈)alkylene(C₆-C₁₀)aryl;
U is O or S;
X is O, S, or NR^{X10};
R^{X10} is hydrogen; or an optionally substituted aliphatic residue or an optionally substituted aromatic residue;
D is a drug moiety;
Y¹ is NR^{A20}, O, S, or CR^{A21}R^{A22};
R^{A20} is selected from the group consisting of hydrogen, (C₁-C₈)alkyl, (C₆-C₁₀)aryl, and C₁-C₈)alkylene(C₆-C₁₀)aryl;
R^{A21} and R^{A22} are each independently selected from the group consisting of hydrogen, (C₁-C₈)alkyl, (C₆-C₁₀)aryl, and (C₁-C₈)alkylene(C₆-C₁₀)aryl;
E is a spacer;
Z is a cleavable group;
W is a moiety which, after cleavage of the group Z, is capable of forming a ring together with the spacer E, Y¹ and the phosphorus; and
n is an integer ranging from 1 to 20.

19. A pharmaceutical composition comprising a conjugate according to claim 1.

20. A method of treating cancer in a subject, said method comprising administering to the subject with cancer a conjugate according to claim 1.


SEQUENCE LISTING (7 sequences)

SEQ ID NO: 1 - protein, 8 aa, unidentified
IWGIGCNP

SEQ ID NO: 2 - protein, 447 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 237 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSVDSVEGEGEEEGEE

SEQ ID NO: 4 - protein, 451 aa, synthetic construct
EVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC